(19) United States
(12) Patent Application Publication
(10) Pub. No.: US 2005/0255568 A1
(43) Pub. Date: Nov. 17, 2005

(54) Methods and compositions for amino acid production

(76) Inventors: Richard B. Bailey, South Natick, MA (US); Paul Blomquist, Roslindale, MA (US); Reed Doten, Framingham, MA (US); Edward M. Driggers, Arlington, MA (US); Kevin T. Madden, Arlington, MA (US); Jessica O'Leary, Somerville, MA (US); George O'Toole, Hanover, NH (US); Joshua Trueheart, Concord, MA (US); Michael J. Walbridge, Dorchester, MA (US); Peter S. Yorgey, Cambridge, MA (US)

Correspondence Address: FISH & RICHARDSON PC, 225 FRANKLIN ST, BOSTON, MA, 02110, US

(21) Appl. No.: 10/858,730

(22) Filed: Jun. 1, 2004

Related U.S. Application Data
(60) Provisional application No. 60/475,000, filed on May 30, 2003.
(60) Provisional application No. 60/551,860, filed on Mar. 10, 2004.

(51) Int. Cl. C12P 13/12; C12N 9/06; C12N 9/10; C12N 1/21

(52) U.S. Cl. 435/113; 435/252.33; 435/191; 435/193

(57) ABSTRACT

Methods and compositions for amino acid production using genetically modified bacteria are disclosed.

### CROSS-REFERENCE TO RELATED APPLICATIONS

This application claims the benefit of priority of U.S. Ser. No. 60/475,000, filed May 30, 2003, and U.S. Ser. No. 60/551,860, filed Mar. 10, 2004. The entire contents of these applications are hereby incorporated by reference.

### TECHNICAL FIELD

This invention relates to microbiology and molecular biology, and more particularly to methods and compositions for amino acid production.

### BACKGROUND

Industrial fermentation of bacteria is used to produce commercially useful metabolites such as amino acids, nucleotides, vitamins, and antibiotics. Many of the bacterial production strains that are used in these fermentation processes have been generated by random mutagenesis and selection of mutants (Demain, A. L. Trends Biotechnol. 18:26-31, 2000). Accumulation of secondary mutations in mutagenized production strains and derivatives of these strains can reduce the efficiency of metabolite production due to altered growth and stress-tolerance properties. The availability of genomic information for production strains and related bacterial organisms provides an opportunity to construct new production strains by the introduction of cloned nucleic acids into naive, unmanipulated host strains, thereby allowing amino acid production in the absence of deleterious mutations (Ohnishi, J., et al. Appl Microbiol Biotechnol. 58:217-223, 2002). Similarly, this information provides an opportunity for identifying and overcoming the limitations of existing production strains.

### SUMMARY

The present invention relates to compositions and methods for production of amino acids and related metabolites in bacteria. In various embodiments, the invention features bacterial strains that are engineered to increase the production of amino acids and related metabolites of the aspartic acid family. The strains can be engineered to harbor one or more nucleic acid molecules (e.g., recombinant nucleic acid molecules) encoding a polypeptide (e.g., a polypeptide that is heterologous or homologous to the host cell) and/or they may be engineered to increase or decrease expression and/or activity of polypeptides (e.g., by mutation of endogenous nucleic acid sequences). These polypeptides, which can be expressed by various methods familiar to those skilled in the art, include variant polypeptides, such as variant polypeptides with reduced feedback inhibition. These variant polypeptides may exhibit reduced feedback inhibition by a product or intermediate of an amino acid biosynthetic pathway, such as S-adenosylmethionine, lysine, threonine or methionine, relative to wild type forms of the proteins. Also featured are the variant polypeptides encoded by the nucleic acids, as well as bacterial cells comprising the nucleic acids and the polypeptides. Combinations of nucleic acids, and cells that include the combinations of nucleic acids, are also provided herein. The invention also relates to improved bacterial production strains, including, without limitation, strains of coryneform bacteria and Enterobacteriaceae (e.g., Escherichia coli (E. coli)).

Bacterial polypeptides that regulate the production of an amino acid from the aspartic acid family of amino acids or related metabolites include, for example, polypeptides involved in the metabolism of methionine, threonine, isoleucine, aspartate, lysine, cysteine and sulfur, such as enzymes that catalyze the conversion of intermediates of amino acid biosynthetic pathways to other intermediates and/or end product, and polypeptides that directly regulate the expression and/or function of such enzymes. The following list is only a partial list of polypeptides involved in amino acid synthesis: homoserine O-acetyltransferase, O-acetylhomoserine sulfhydrylase, methionine adenosyltransferase, cystathionine beta-lyase, O-succinylhomoserine (thio)-lyase/O-acetylhomoserine (thio)-lyase, the McbR gene product, homocysteine methyltransferase, aspartokinases, pyruvate carboxylase, phosphoenolpyruvate carboxylase, aspartate aminotransferase, aspartate semialdehyde dehydrogenase, homoserine dehydrogenase, dihydrodipicolinate synthase, dihydrodipicolinate reductase, N-succinyl-LL-diaminopimelate aminotransferase, tetrahydrodipicolinate N-succinyltransferase, N-succinyl-LL-diaminopimelate desuccinylase, diaminopimelate epimerase, diaminopimelate decarboxylase, diaminopimelate dehydrogenase, glutamate dehydrogenase, 5-methyltetrahydropteroyltriglutamate-homocysteine methyltransferase, serine hydroxymethyltransferase, 5,1 0-methylenetetrahydrofolate reductase, serine O-acetyltransferase, D-3-phosphoglycerate dehydrogenase, and homoserine kinase.

Heterologous proteins may be encoded by genes of any bacterial organism other than the host bacterial species. The heterologous genes can be genes from the following, non-limiting list of bacteria: Mycobacterium smegmatis; Amycolatopsis mediterranei; Streptomyces coelicolor; Thermobifida fusca; Erwinia chrysanthemi; Shewanella oneidensis; Lactobacillus plantarum; Bifidobacterium longum; Bacillus sphaericus; and Pectobacterium chrysanthemi. Of course, heterologous genes for host strains from the Enterobacteriaceae family also include genes from coryneform bacteria. Likewise, heterologous genes for host strains of coryneform bacteria also include genes from Enterobacteriaceae family members. In certain embodiments, the host strain is Escherichia coli and the heterologous gene is a gene of a species other than a coryneform bacteria. In certain embodiments, the host strain is a coryneform bacteria and the heterologous gene is a gene of a species other than Escherichia coli. In certain embodiments, the host strain is Escherichia coli and the heterologous gene is a gene of a species other than Corynebacterium glutamicum. In certain embodiments, the host strain is Corynebacterium glutamicum and the heterologous gene is a gene of a species other than Escherichia coli.

In various embodiments, the polypeptide is encoded by a gene obtained from an organism of the order Actinomycetales. In various embodiments, the heterologous nucleic acid molecule is obtained from Mycobacterium smegmatis, Streptomyces coelicolor, Thermobifida fusca, Amycolatopsis mediterranei, or a coryneform bacteria. In various embodiments, the heterologous protein is encoded by a gene obtained from an organism of the family Enterobacteriaceae. In various embodiments, the heterologous nucleic acid molecule is obtained from Erwinia chysanthemi or Escherichia coli.

In various embodiments, the host bacterium (e.g., coryneform bacterium or bacterium of the family Enterobacteriaceae) also has increased levels of a polypeptide encoded by a gene from the host bacterium (e.g., from a coryneform bacterium or a bacterium of the family Enterobacteriaceae such as an Escherichia coli bacterium). Increased levels of a polypeptide encoded by a gene from the host bacterium may result from one of the following: introduction of additional copies of a gene from the host bacterium under the naturally occurring promoter; introduction of additional copies of a gene from the host bacterium under the control of a promoter, e.g., a promoter more optimal for amino acid production than the naturally occurring promoter, either from the host or a heterologous organism; or the replacement of the naturally occurring promoter for the gene from the host bacterium with a promoter more optimal for amino acid production, either from the host or a heterologous organism. Vectors used to generate increased levels of a protein may be integrated into the host genome or exist as an episomal plasmid.

In various embodiments, the host bacterium has reduced activity of a polypeptide (e.g., a polypeptide involved in amino acid synthesis, e.g., an endogenous polypeptide) (e.g., decreased relative to a control). Reducing the activity of particular polypeptides involved in amino acid synthesis can facilitate enhanced production of particular amino acids and related metabolites. In one embodiment, expression of a dihydrodipicolinate synthase polypeptide is deficient in the bacterium (e.g., an endogenous dapA gene in the bacterium is mutated or deleted). In various embodiments, expression of one or more of the following polypeptides is deficient: an mcbR gene product, homoserine dehydrogenase, homoserine kinase, methionine adenosyltransferase, homoserine O-acetyltransferase, and phosphoenolpyruvate carboxykinase.

In various embodiments the nucleic acid molecule comprises a promoter, including, for example, the lac, trc, trcRBS, phoA, tac, or λP_{L}/λP_{R} promoter from E. coli (or derivatives thereof) or the phoA, gpd, rplM, or rpsJ promoter from a coryneform bacteria.

In one aspect, the invention features a host bacterium (e.g., a coryneform bacterium or a bacterium of the family Enterobacteriaceae such as an Escherichia coli bacterium) comprising at least one (two, three, or four) of: (a) a nucleic acid molecule comprising a sequence encoding a heterologous bacterial aspartokinase polypeptide or a functional variant thereof; (b) a nucleic acid molecule comprising a sequence encoding a heterologous bacterial aspartate semialdehyde dehydrogenase polypeptide or a functional variant thereof; (c) a nucleic acid molecule comprising a sequence encoding a heterologous bacterial phosphoenolpyruvate carboxylase polypeptide or a functional variant thereof; (d) a nucleic acid molecule comprising a sequence encoding a heterologous bacterial pyruvate carboxylase polypeptide or a functional variant thereof; (e) a nucleic acid molecule comprising a sequence encoding a heterologous bacterial dihydrodipicolinate synthase polypeptide or a functional variant thereof; (f) a nucleic acid molecule comprising a sequence encoding a heterologous bacterial homoserine dehydrogenase polypeptide or a functional variant thereof; (g) a nucleic acid molecule comprising a sequence encoding a heterologous bacterial homoserine O-acetyltransferase polypeptide or a functional variant thereof; (h) a nucleic acid molecule comprising a sequence encoding a heterologous bacterial O-acetylhomoserine sulfhydrylase polypeptide or a functional variant thereof; (i) a nucleic acid molecule comprising a sequence encoding a heterologous bacterial methionine adenosyltransferase polypeptide or a functional variant thereof; (j) a nucleic acid molecule comprising a sequence encoding a heterologous bacterial mcbR gene product polypeptide or a functional variant thereof; (k) a nucleic acid molecule comprising a sequence encoding a heterologous bacterial O-succinylhomoserine/acetylhomoserine (thiol)-lyase polypeptide or a functional variant thereof; (l) a nucleic acid molecule comprising a sequence encoding a heterologous bacterial cystathionine beta-lyase polypeptide or a functional variant thereof; (m) a nucleic acid molecule comprising a sequence encoding a heterologous bacterial 5-methyltetrahydrofolate homocysteine methyltransferase polypeptide or a functional variant thereof; and (n) a nucleic acid molecule comprising a sequence encoding a heterologous bacterial 5-methyltetrahydropteroyltriglutamate-homocysteine methyltransferase polypeptide or a functional variant thereof.

In various embodiments, the nucleic acid molecule is an isolated nucleic acid molecule (e.g., the nucleic acid molecule is free of nucleotide sequences that naturally flank the sequence in the organism from which the nucleic acid molecule is derived, e.g., the nucleic acid molecule is a recombinant nucleic acid molecule).

In various embodiments, the bacterium comprises nucleic acid molecules comprising sequences encoding two or more distinct heterologous bacterial polypeptides, wherein each of the heterologous polypeptides encodes the same type of polypeptide (e.g., the bacterium comprises nucleic acid molecules comprising sequences encoding an aspartokinase from a first species, and sequences encoding an aspartokinase from a second species.)

In various embodiments, the polypeptide is selected from an Enterobacteriaceae polypeptide, an Actinomycetes polypeptide, or a variant thereof. In various embodiments, the polypeptide is a polypeptide of one of the following Actinomycetes species: Mycobacterium smegmatis, Streptomyces coelicolor, Thermobifida fusca, Amycolatopsis mediterranei and coryneform bacteria, including Corynebacterium glutamicum. In various embodiments, the polypeptide is a polypeptide of one of the following Enterobacteriaceae species: Erwinia chysanthemi and Escherichia coli.

In various embodiments, the polypeptide is a variant polypeptide with reduced feedback inhibition (e.g., relative to a wild-type form of the polypeptide). In various embodiments, the bacterium further comprises additional heterologous bacterial gene products involved in amino acid production. In various embodiments, the bacterium further comprises a nucleic acid molecule encoding a heterologous bacterial polypeptide described herein (e.g., a nucleic acid molecule encoding a heterologous bacterial homoserine dehydrogenase polypeptide). In various embodiments, the bacterium further comprises a nucleic acid molecule encoding a homologous bacterial polypeptide (i.e., a bacterial polypeptide that is native to the host species or a functional variant thereof), such as a bacterial polypeptide described herein. The homologous bacterial polypeptide can be expressed at high levels and/or conditionally expressed. For example, the nucleic acid encoding the homologous bacterial polypeptide can be operably linked to a promoter that allows expression of the polypeptide over wild-type levels, and/or the nucleic acid may be present in multiple copies in the bacterium.

In various embodiments the heterologous bacterial aspartokinase or functional variant thereof is chosen from: (a) a Mycobacterium smegmatis aspartokinase polypeptide or a functional variant thereof, (b) an Amycolatopsis mediterranei aspartokinase polypeptide or a functional variant thereof, (c) a Streptomyces coelicolor aspartokinase polypeptide or a functional variant thereof, (d) a Thermobifidafusca aspartokinase polypeptide or a functional variant thereof, (e) an Erwinia chrysanthemi aspartokinase polypeptide or a functional variant thereof, and (f) a Shewanella oneidensis aspartokinase polypeptide or a functional variant thereof. In certain embodiments, the heterologous bacterial aspartokinase polypeptide is an Escherichia coli aspartokinase polypeptide or a functional variant thereof. In certain embodiments, the heterologous bacterial aspartokinase polypeptide is a Corynebacterium glutamicum aspartokinase polypeptide or a functional variant thereof. In certain embodiments the heterologous bacterial asparatokinase polypeptide or functional variant thereof has reduced feedback inhibition.

In various embodiments the heterologous bacterial aspartate semialdehyde dehydrogenase polypeptide or functional variant thereof is chosen from: (a) a Mycobacterium smegmatis aspartate semialdehyde dehydrogenase polypeptide r a functional variant thereof, (b) an Amycolatopsis mediterranei asp artate semi aldehyde dehydrogenase polypeptide or a functional variant thereof, (c) a Streptomyces coelicolor aspartate semialdehyde dehydrogenase polypeptide or a functional variant thereof, and (d) a Thermobifida fusca aspartate semialdehyde dehydrogenase polypeptide or a functional variant thereof. In certain embodiments, the heterologous bacterial aspartate semialdehyde dehydrogenase polypeptide is an Escherichia coli aspartate semialdehyde dehydrogenase polypeptide or a functional variant thereof. In certain embodiments, the heterologous bacterial aspartate semialdehyde dehydrogenase polypeptide is a Corynebacterium glutamicum aspartate semialdehyde dehydrogenase polypeptide or a functional variant thereof. In various embodiments the heterologous bacterial phosphoenolpyruvate carboxylase polypeptide or functional variant thereof is chosen from: (a) a Mycobacterium smegmatis phosphoenolpyruvate carboxylase polypeptide or a functional variant thereof, (b) a Streptomyces coelicolor phosphoenolpyruvate carboxylase polypeptide or a functional variant thereof, (c) a Thermobifida fusca phosphoenolpyruvate carboxylase polypeptide or a functional variant thereof, and (d) an Erwinia chrysanthemi phosphoenolpyruvate carboxylase polypeptide or a functional variant thereof. In certain embodiments, the heterologous bacterial phosphoenolpyruvate carboxylase polypeptide is an Escherichia coli phosphoenolpyruvate carboxylase polypeptide or a functional variant thereof. In certain embodiments, the heterologous bacterial phosphoenolpyruvate carboxylase polypeptide is a Corynebacterium glutamicum phosphoenolpyruvate carboxylase polypeptide or a functional variant thereof.

In various embodiments the heterologous bacterial pyruvate carboxylase polypeptide or functional variant thereof is chosen from: (a) a Mycobacterium smegmatis pyruvate carboxylase polypeptide or a functional variant thereof, (b) a Streptomyces coelicolor pyruvate carboxylase polypeptide or a functional variant thereof, and (c) a Thermobifida fusca pyruvate carboxylase polypeptide or a functional variant thereof. In certain embodiments, the heterologous bacterial pyruvate carboxylase polypeptide is a Corynebacterium glutamicum pyruvate carboxylase or a functional variant thereof.

In various embodiments the bacterium is chosen from a coryneform bacterium or a bacterium of the family Enterobacteriaceae such as an Escherichia coli bacterium. Coryneform bacteria include, without limitation, Corynebacterium glutamicum, Corynebacterium acetoglutamicum, Corynebacterium melassecola, Corynebacterium thermoaminogenes, Brevibacterium lactofermentum, Brevibacterium lactis, and Brevibacterium flavum.

In various embodiments: the Mycobacterium smegmatis aspartokinase polypeptide comprises SEQ ID NO: 1 or a variant sequence thereof, the Amycolatopsis mediterranei aspartokinase polypeptide comprises SEQ ID NO:2 or a variant sequence thereof, the Streptomyces coelicolor aspartokinase polypeptide comprises SEQ ID NO:3 or a variant sequence thereof, the Thermobifida fusca aspartokinase polypeptide comprises SEQ ID NO:4 or a variant sequence thereof, the Erwinia chrysanthemi aspartokinase polypeptide comprises SEQ ID NO:5 or a variant sequence thereof, and the Shewanella oneidensis aspartokinase polypeptide comprises SEQ ID NO:6 or a variant sequence thereof, the Escherichia coli aspartokinase polypeptide comprises SEQ ID NO: 203 or a variant sequence thereof, the Corynebacterium glutamicum aspartokinase polypeptide comprises SEQ ID NO: 202 or a variant sequence thereof, the Corynebacterium glutamicum aspartate semialdehyde dehydrogenase polypeptide comprises SEQ ID NO:204 or a variant sequence thereof, the Escherichia coli aspartate semialdehyde dehydrogenase polypeptide comprises SEQ ID NO: 205 or a variant sequence thereof, the Mycobacterium smegmatis phosphoenolpyruvate carboxylase polypeptide or functional variant thereof comprises an amino acid sequence at least 80% identical to SEQ ID NO:8 (M. leprae phosphoenolpyruvate carboxylase) (e.g., a sequence at least 80%, 85%, 90%, 92%, 94%, 95%, 96%, 97%, 98%, 99% or more identical to SEQ ID NO:8), the Streptomyces coelicolor phosphoenolpyruvate carboxylase polypeptide comprises SEQ ID NO:9 or a variant sequence thereof, the Thermobifida fusca phosphoenolpyruvate carboxylase polypeptide comprises SEQ ID NO:7 or a variant sequence thereof, the Erwinia chrysanthemi phosphoenolpyruvate carboxylase polypeptide comprises SEQ ID NO:10 or a variant sequence thereof, the Mycobacterium smegmatis pyruvate carboxylase polypeptide comprises SEQ ID NO:13 or a variant sequence thereof, the Streptomyces coelicolor pyruvate carboxylase polypeptide comprises SEQ ID NO: 12 or a variant sequence thereof, and the Corynebacterium glutamicum pyruvate carboxylase polypeptide comprises SEQ ID NO:208 or a variant sequence thereof.

In various embodiments, the Mycobacterium smegmatis aspartokinase polypeptide comprises at least one amino acid change chosen from: an alanine changed to a Group 1 amino acid residue at position 279; a serine changed to a Group 6 amino acid residue at position 301; a threonine changed to a Group 2 amino acid residue at position 311; and a glycine changed to a Group 3 amino acid residue at position 345; the Mycobacterium smegmatis aspartokinase comprises at least one amino acid change chosen from: an alanine changed to a proline at position 279, a serine changed to a tyrosine at position 301, a threonine changed to an isoleucine at position 311, and a glycine changed to an aspartate at position 345.

In various embodiments, the Amycolatopsis mediterranei aspartokinase polypeptide comprises at least one amino acid change chosen from: an alanine changed to a Group 1 amino acid residue at position 279; a serine changed to a Group 6 amino acid residue at position 301 ;a threonine changed to a Group 2 amino acid residue at position 311; and a glycine changed to a Group 3 amino acid residue at position 345.

In various embodiments the Amycolatopsis mediterranei aspartokinase polypeptide comprises at least one amino acid change chosen from: an alanine changed to a proline at position 279; a serine changed to a tyrosine at position 301; a threonine changed to an isoleucine at position 311; and a glycine changed to an aspartate at position 345.

In various embodiments the Streptomyces coelicolor aspartokinase polypeptide comprises at least one amino acid change chosen from: an alanine changed to a Group 1 amino acid residue at position 282; a serine changed to a Group 6 amino acid residue at position 304; a serine changed to a Group 2 amino acid residue at position 314; and a glycine changed to a Group 3 amino acid residue at position 348.

In various embodiments the Streptomyces coelicolor aspartokinase polypeptide comprises at least one amino acid change chosen from: an alanine changed to a proline at position 282; a serine changed to a tyrosine at position 304; a serine changed to an isoleucine at position 314; and a glycine changed to an aspartate at position 348.

In various embodiments the Erwinia chrysanthemi aspartokinase polypeptide comprises at least one amino acid change chosen from: a glycine changed to a Group 3 amino acid residue at position 328; a leucine changed to a Group 6 amino acid residue at position 330; a serine changed to a Group 2 amino acid residue at position 350; and a valine changed to a Group 2 amino acid residue other than valine at position 352.

In various embodiments the Erwinia chrysanthemi aspartokinase polypeptide comprises at least one amino acid change chosen from: a glycine changed to an aspartate at position 328; a leucine changed to a phenylalanine at position 330; a serine changed to an isoleucine at position 350; and a valine changed to a methionine at position 352.

In various embodiments the Shewanella oneidensis aspartokinase polypeptide comprises at least one amino acid change chosen from: a glycine changed to a Group 3 amino acid residue at position 323; a leucine changed to a Group 6 amino acid residue at position 325; a serine changed to a Group 2 amino acid residue at position 345; and a valine changed to a Group 2 amino acid residue other than valine at position 347.

In various embodiments the Shewanella oneidensis aspartokinase polypeptide comprises at least one amino acid change chosen from: a glycine changed to an aspartate at position 323; a leucine changed to a phenylalanine at position 325; a serine changed to an isoleucine at position 345; and a valine changed to a methionine at position 347.

In various embodiments the Corynebacterium glutamicum aspartokinase polypeptide comprises at least one amino acid change chosen from: an alanine changed to a Group 1 amino acid other than alanine at position 279; a serine changed to a Group 6 amino acid residue at position 301; a threonine changed to a Group 2 amino acid residue at position 311; and a glycine changed to a Group 3 amino acid residue at position 345.

In various embodiments the Corynebacterium glutamicum aspartokinase polypeptide comprises at least one amino acid change chosen from: an alanine changed to a proline at position 279; a serine changed to a tyrosine at position 301; a threonine changed to an isoleucine at position 311; and a glycine changed to an aspartate at position 345.

In various embodiments the Escherichia coli aspartokinase polypeptide comprises at least one amino acid change chosen from: a glycine changed to a Group 3 amino acid residue at position 323; a leucine changed to a Group 6 amino acid residue at position 325; a serine changed to a Group 2 amino acid residue at position 345; and a valine changed to a Group 2 amino acid residue other than valine at position 347.

In various embodiments the Escherichia coli aspartokinase polypeptide comprises at least one amino acid change chosen from: a glycine changed to an aspartate at position 323; a leucine changed to a phenylalanine at position 325; a serine changed to an isoleucine at position 345; and a valine changed to a methionine at position 347.

In various embodiments, the Corynebacterium glutamicum pyruvate carboxylase polypeptide or variant thereof comprises a proline changed to Group 4 amino acid residue at position 458. In various embodiments, the Corynebacterium glutamicum pyruvate carboxylase polypeptide or variant thereof comprises a proline changed to a serine at position 458.

In various embodiments, the Mycobacterium smegmatis pyruvate carboxylase polypeptide or variant thereof comprises a proline changed to Group 4 amino acid residue at position 448. In various embodiments, the Mycobacterium smegmatis pyruvate carboxylase polypeptide or variant thereof comprises a proline changed to a serine at position 448.

In various embodiments, the Streptomyces coelicolor pyruvate carboxylase polypeptide or variant thereof comprises a proline changed to Group 4 amino acid residue at position 449. In various embodiments, the Streptomyces coelicolor pyruvate carboxylase polypeptide or variant thereof comprises a proline changed to a serine at position 449.

The invention also features a coryneform bacterium or a bacterium of the family Enterobacteriaceae such as an Escherichia coli bacterium comprising a nucleic acid molecule that encodes a heterologous bacterial dihydrodipicolinate synthase or a functional variant thereof.

In various embodiments the heterologous bacterial dihydrodipicolinate synthase polypeptide or functional variant thereof is chosen from: a Mycobacterium smegmatis dihydrodipicolinate synthase polypeptide or a functional variant thereof; a Streptomyces coelicolor dihydrodipicolinate synthase polypeptide or a functional variant thereof; a Thermobifida fusca dihydrodipicolinate synthase polypeptide or a functional variant thereof; and an Erwinia chrysanthemi dihydrodipicolinate synthase polypeptide or a functional variant thereof. In certain embodiments, the heterologous bacterial dihydrodipicolinate synthase polypeptide or functional variant thereof with reduced feedback inhibition is an Escherichia coli dihydrodipicolinate synthase polypeptide or a functional variant thereof. In certain embodiments the heterologous bacterial dihydrodipicolinate synthase polypeptide or functional variant thereof has reduced feedback inhibition.

In various embodiments, the Mycobacterium smegmatis dihydrodipicolinate synthase polypeptide is at least 80% identical to SEQ ID NO:15 or SEQ ID NO:16 (e.g., a sequence at least 80%, 85%, 90%, 92%, 94%, 95%, 96%, 97%, 98%, 99% or more identical to SEQ ID NO: 15 or SEQ ID NO: 16); the Streptomyces coelicolor dihydrodipicolinate synthase polypeptide comprises SEQ ID NO: 17 or a variant sequence thereof; the Thermobifida fusca dihydrodipicolinate synthase polypeptide comprises SEQ ID NO: 14 or a variant sequence thereof; and the Erwinia chrysanthemi dihydrodipicolinate synthase polypeptide comprises SEQ ID NO: 18 or a variant sequence thereof.

In various embodiments the Erwinia chrysanthemi dihydrodipicolinate synthase polypeptide comprises at least one amino acid change chosen from: an asparagine changed to a Group 2 amino acid residue at position 80; a leucine changed to a Group 6 amino acid residue at position 88; and a histidine changed to a Group 6 amino acid residue at position 118.

In various embodiments the Erwinia chrysanthemi dihydrodipicolinate synthase polypeptide comprises at least one amino acid change chosen from: an asparagine changed to an isoleucine at position 80; a leucine changed to a phenylalanine at position 88; and a histidine changed to a tyrosine at position 118.

In various embodiments, the Streptomyces coelicolor dihydrodipicolinate synthase polypeptide comprises at least one amino acid change chosen from: an asparagine changed to a Group 2 amino acid residue at position 89; a leucine changed to a Group 6 amino acid residue at position 97; and a histidine changed to a Group 6 amino acid residue at position 127.

In various embodiments the Streptomyces coelicolor dihydrodipicolinate synthase polypeptide comprises at least one amino acid change chosen from: an asparagine changed to an isoleucine at position 89; a leucine changed to a phenylalanine at position 97; and a histidine changed to a tyrosine at position 127.

In various embodiments the Mycobacterium smegmatis dihydrodipicolinate synthase polypeptide comprises at least one amino acid change chosen from: an amino acid residue corresponding to tyrosine 90 of SEQ ID NO: 16 changed to a Group 2 amino acid residue; an amino acid residue corresponding to leucine 98 of SEQ ID NO: 16 changed to a Group 6 amino acid residue; and an amino acid residue corresponding to histidine 128 of SEQ ID NO:16 changed to a Group 6 amino acid residue.

In various embodiments the Mycobacterium smegmatis dihydrodipicolinate synthase polypeptide comprises at least one amino acid change chosen from: an amino acid residue corresponding to tyrosine 90 of SEQ ID NO:16 changed to an isoleucine; an amino acid residue corresponding to leucine 98 of SEQ ID NO: 16 changed to a phenylalanine; and an amino acid residue corresponding to histidine 128 of SEQ ID NO:16 changed to a histidine.

In various embodiments the Escherichia coli dihydrodipicolinate synthase polypeptide comprises at least one amino acid change chosen from: an asparagine changed to a Group 2 amino acid residue at position 80; an alanine changed to a Group 2 amino acid residue at position 81; a glutamatate changed to a Group 5 amino acid residue at position 84; a leucine changed to a Group 6 amino acid residue at position 88; and a histidine changed to a Group 6 amino acid at position 118.

In various embodiments the Escherichia coli dihydrodipicolinate synthase polypeptide comprises at least one amino acid change chosen from: an asparagine changed to an isoleucine at position 80; an alanine changed to a valine at position 81; a glutamate changed to a lysine at position 84; a leucine changed to a phenylalanine at position 88; and a histidine changed to a tyrosine at position 118. 378; and an alteration that truncates the homoserine dehydrogenase protein after the lysine amino acid residue at position 428. In one embodiment, the Corynebacterium glutamicum or Brevibacterium lactofermentum homoserine dehydrogenase polypeptide is encoded by the hom^{dr} sequence described in WO93/09225 SEQ ID NO. 3.

In various embodiments the Corynebacterium glutamicum or Brevibacterium lactofermentum homoserine dehydrogenase polypeptide comprises at least one amino acid change chosen from: a leucine changed to a phenylalanine at position 23; valine changed to an alanine at position 59; a valine changed to an isoleucine at position 104; and a glycine changed to a glutamic acid at position 378.

In various embodiments the Mycobacterium smegmatis homoserine dehydrogenase polypeptide comprises at least one amino acid change chosen from: a valine change to a Group 6 amino acid residue at position 10; a valine changed to a Group 1 amino acid residue at position 46; and a glycine changed to Group 3 amino acid residue at position 364.

In various embodiments the Mycobacterium smegmatis homoserine dehydrogenase polypeptide comprises at least one amino acid change chosen from: a valine changed to a phenylalanine at position 10; valine changed to an alanine at position 46; and a glycine changed to a glutamic acid at position 378.

In various embodiments the Streptomyces coelicolor homoserine dehydrogenase polypeptide comprises at least one amino acid change chosen from: a leucine change to a Group 6 amino acid residue at position 10; a valine changed to a Group 1 amino acid residue at position 46; a glycine changed to Group 3 amino acid residue at position 362; an alteration that truncates the homoserine dehydrogenase protein after the arginine amino acid residue at position 412In various embodiments the Streptomyces coelicolor homoserine dehydrogenase polypeptide comprises at least one amino acid change chosen from: a leucine changed to a phenylalanine at position 10; a valine changed to an alanine at position 46; and a glycine changed to a glutamic acid at position 362.

In various embodiments the Thermobifida fusca homoserine dehydrogenase polypeptide comprises at least one amino acid change chosen from: a leucine change to a Group 6 amino acid residue at position 192; a valine changed to a Group 1 amino acid residue at position 228; a glycine changed to Group 3 amino acid residue at position 545. In various embodiments, the Thermobifida fusca homoserine dehydrogenase polypeptide is truncated after the arginine amino acid residue at position 595.

In various embodiments the Thermobifida fusca homoserine dehydrogenase polypeptide comprises at least one amino acid change chosen from: a leucine changed to a phenylalanine at 5 position 192; valine changed to an alanine at position 228; and a glycine changed to a glutamic acid at position 545.

In various embodiments the Escherichia coli homoserine dehydrogenase polypeptidecomprises at least one amino acid change in SEQ ID NO:211 chosen from: a glycine changed to a Group 3 amino acid residue at position 330; and a serine changed to a Group 6 amino acid residue at position 352.

In various embodiments the Escherichia coli homoserine dehydrogenase polypeptide comprises at least one amino acid change in SEQ ID NO:211, ,chosen from: a glycine changed to an aspartate at position 330; and a serine changed to a phenylalanine at position 352.

The invention also features: a coryneform bacterium or a bacterium of the family Enterobacteriaceae such as an Escherichia coli bacterium comprising a nucleic acid that encodes a heterologous bacterial O-homoserine acetyltransferase polypeptide or a functional variant thereof.

In various embodiments the heterologous bacterial O-homoserine acetyltransferase polypeptide is chosen from: a Mycobacterium smegmatis O-homoserine acetyltransferase polypeptide or functional variant thereof; a Streptomyces coelicolor O-homoserine acetyltransferase polypeptide or a functional variant thereof; a Thermobifida fusca O-homoserine acetyltransferase polypeptide or a functional variant thereof; and an Erwinia chrysanthemi O-homoserine acetyltransferase polypeptide or a functional variant thereof. In certain embodiments, the heterologous bacterial O-homoserine acetyltransferase polypeptide is an O-homoserine acetyltransferase polypeptide from Corynebacterium glutamicum or a functional variant thereof. In certain embodiments the heterologous O-homoserine acetyltransferase polypeptide or functional variant thereof has reduced feedback inhibition. In various embodiments the Mycobacterium smegmatis O-homoserine acetyltransferase polypeptide is at least 80% identical to SEQ ID NO:22 or SEQ ID NO:23 (e.g., a sequence at least 80%, 85%, 30 90%, 92%, 94%, 95%, 96%, 97%, 98%, 99% or more identical to SEQ ID NO:22 or SEQ ID NO:23); the heterologous bacterial O-homoserine acetyltransferase polypeptide is a

The invention also features a coryneform bacterium or a bacterium of the family Enterobacteriaceae such as an Escherichia coli bacterium comprising a nucleic acid molecule that encodes a heterologous bacterial homoserine dehydrogenase or a functional variant thereof.

In various embodiments the heterologous bacterial homoserine dehydrogenase polypeptide is chosen from: (a) a Mycobacterium smegmatis homoserine dehydrogenase polypeptide or functional variant thereof; (b) a Streptomyces coelicolor homoserine dehydrogenase polypeptide or a functional variant thereof; (c) a Thermobifida fusca homoserine dehydrogenase polypeptide or a functional variant thereof; and (d) an Erwinia chrysanthemi homoserine dehydrogenase polypeptide or a functional variant thereof. In certain embodiments, the heterologous bacterial homoserine dehydrogenase polypeptide is a homoserine dehydrogenase polypeptide from a coryneform bacteria or a functional variant thereof (e.g., a Corynebacterium glutamicum homoserine dehydrogenase polypeptide or functional variant thereof, or a Brevibacterium lactofermentum homoserine dehydrogenase polypeptide or functional variant thereof). In certain embodiments, the heterologous homoserine dehydrogenase polypeptide or functional variant thereof is an Escherichia coli homoserine dehydrogenase polypeptide or a functional variant thereof. In certain embodiments the heterologous homoserine dehydrogenase polypeptide or functional variant thereof has reduced feedback inhibition.

In various embodiments the heterologous bacterial homoserine dehydrogenase polypeptide is a Streptomyces coelicolor homoserine dehydrogenase polypeptide or functional variant thereof with reduced feedback inhibition; the Streptomyces coelicolor homoserine dehydrogenase polypeptide comprises SEQ ID NO: 19 or a variant sequence thereof; the Thermobifida fusca homoserine dehydrogenase polypeptide comprises SEQ ID NO:21 or a variant sequence thereof; the Corynebacterium glutamicum and Brevibacterium lactofermentum homoserine dehydrogenases polypeptide comprise SEQ ID NO:209 or a variant sequence thereof; and the Escherichia coli homoserine dehydrogenase polypeptide comprises either SEQ ID NO:210, SEQ ID NO:21 1, or a variant sequence thereof

In various embodiments the Corynebacterium glutamicum or Brevibacterium lactofermentum homoserine dehydrogenase polypeptide comprises at least one amino acid change chosen from: a leucine change to a Group 6 amino acid residue at position 23; a valine changed to a Group 1 amino acid residue at position 59; a valine changed to another Group 2 amino acid residue at position 104; a glycine changed to Group 3 amino acid residue at position Thermobifida fusca O-homoserine acetyltransferase polypeptide or functional variant thereof; the Thermobifida fusca O-homoserine acetyltransferase polypeptide comprises SEQ ID NO:24 or a variant sequence thereof; the heterologous bacterial O-homoserine acetyltransferase polypeptide is a Corynebacterium glutamicum O-homoserine acetyltransferase polypeptide or functional variant thereof; the C. glutamicum O-homoserine acetyltransferase polypeptide comprises SEQ ID NO:212 or a variant sequence thereof; or the heterologous bacterial O-homoserine acetyltransferase polypeptide is a Escherichia coli O-homoserine acetyltransferase polypeptide or functional variant thereof; the Escherichia coli O-homoserine acetyltransferase polypeptide comprises SEQ ID NO:213 or a variant sequence thereof.

The invention also features a coryneform bacterium or a bacterium of the family Enterobacteriaceae such as an Escherichia coli bacterium comprising a nucleic acid molecule that encodes a heterologous bacterial O-acetylhomoserine sulfhydrylase or a functional variant thereof.

In various embodiments the heterologous bacterial O-acetylhomoserine sulfhydrylase polypeptide is chosen from: (a) a Mycobacterium smegmatis O-acetylhomoserine sulfhydrylase polypeptide or functional variant thereof; (b) a Streptomyces coelicolor O-acetylhomoserine sulfhydrylase polypeptide or a functional variant thereof; and (c) a Thermobifida fusca O-acetylhomoserine sulfhydrylase polypeptide or a functional variant thereof. In certain embodiments, the heterologous bacterial O-acetylhomoserine sulffiydrylase polypeptide is an O-acetylhomoserine sulfhydrylase polypeptide from Corynebacterium glutamicum or a functional variant thereof. In certain embodiments the heterologous O-acetylhomoserine sulfhydrylase polypeptide or functional variant thereof has reduced feedback inhibition.

In various embodiments the Mycobacterium smegmatis O-acetylhomoserine sulfhydrylase polypeptide is at least 80% identical to SEQ ID NO:26 (e.g., a sequence at least 80%, 85%, 90%, 92%, 94%, 95%, 96%, 97%, 98%, 99% or more identical to SEQ ID NO:26); the Thermobifida fusca O-acetylhomoserine sulfhydrylase polypeptide comprises SEQ ID NO:25 or a variant sequence thereof; and the Corynebacterium glutamicum heterologous bacterial O-acetylhomoserine sulfhydrylase polypeptide comprises SEQ ID NO:214 or a variant sequence thereof.

The invention also features a coryneform bacterium or a bacterium of the family Enterobacteriaceae such as an Escherichia coli bacterium comprising a nucleic acid molecule that encodes a heterologous bacterial methionine adenosyltransferase or a functional variant thereof.

In various embodiments the heterologous bacterial methionine adenosyltransferase polypeptide is chosen from: a Mycobacterium smegmatis methionine adenosyltransferase polypeptide or functional variant thereof; a Streptomyces coelicolor methionine adenosyltransferase polypeptide or a functional variant thereof; a Thermobifida fusca methionine adenosyltransferase polypeptide or a functional variant thereof; and an Erwinia chrysanthemi methionine adenosyltransferase polypeptide or a functional variant thereof. In certain embodiments, the heterologous bacterial methionine adenosyltransferase polypeptide is a methionine adenosyltransferase polypeptide from Corynebacterium glutamicum or a functional variant thereof. In certain embodiments, the heterologous bacterial methionine adenosyltransferase polypeptide is a methionine adenosyltransferase polypeptide from Escherichia coli or a functional variant thereof. In certain embodiments the heterologous methionine adenosyltransferase polypeptide or functional variant thereof has reduced feedback inhibition In various embodiments the Mycobacterium smegmatis O-methionine adenosyltransferase polypeptide is at least 80% identical to SEQ ID NO:27 or SEQ ID NO:28 (e.g., a sequence at least 80%, 85%, 90%, 92%, 94%, 95%, 96%, 97%, 98%, 99% or more identical to SEQ ID NO:27 or SEQ ID NO:28); the Streptomyces coelicolor methionine adenosyltransferase polypeptide comprises SEQ ID NO:30 or a variant sequence thereof; the heterologous bacterial methionine adenosyltransferase polypeptide is a Thermobifida fusca methionine adenosyltransferase or functional variant thereof; the Thermobifida fusca methionine adenosyltransferase polypeptide comprises SEQ ID NO:29 or a variant sequence thereof; the Corynebacterium glutamicum heterologous bacterial methionine adenosyltransferase comprises SEQ ID NO:215 or a variant sequence thereof; and the Escherichia coli heterologous bacterial methionine adenosyltransferase polypeptide comprises SEQ ID NO:216 or a variant sequence thereof.

In various embodiments the bacterium further comprises a nucleic acid molecule encoding a heterologous bacterial dihydrodipicolinate synthase polypeptide or a functional variant thereof.

In various embodiments the heterologous bacterial dihydrodipicolinate synthase polypeptide or a functional variant thereof is chosen from: a Mycobacterium smegmatis dihydrodipicolinate synthase polypeptide or a functional variant thereof; a Streptomyces coelicolor dihydrodipicolinate synthase polypeptide or a functional variant thereof; a Thermobifida fusca dihydrodipicolinate synthase polypeptide or a functional variant thereof; an Erwinia chrysanthemi dihydrodipicolinate synthase polypeptide or a functional variant thereof; an Escherichia coli dihydrodipicolinate synthase polypeptide or a functional variant thereof; and a Corynebacterium glutamicum dihydrodipicolinate synthase polypeptide or a functional variant thereof. In certain embodiments the heterologous dihydrodipicolinate synthase polypeptide or functional variant thereof has reduced feedback inhibition.

In various embodiments the bacterium further comprises at least one of: (a) a nucleic acid molecule encoding a heterologous bacterial homoserine dehydrogenase polypeptide or a functional variant thereof; (b) a nucleic acid molecule encoding a heterologous bacterial O-homoserine acetyltransferase polypeptide or a functional variant thereof; (c) a nucleic acid molecule encoding a heterologous O-acetylhomoserine sulfhydrylase polypeptide or a functional variant thereof. In certain embodiments one or more of the heterologous polypeptides or functional variants thereof has reduced feedback inhibition.

In various embodiments the heterologous bacterial homoserine dehydrogenase polypeptide is chosen from: a Mycobacterium smegmatis homoserine dehydrogenase polypeptide or functional variant thereof; a Streptomyces coelicolor homoserine dehydrogenase polypeptide or a functional variant thereof; a Thermobifida fusca homoserine dehydrogenase polypeptide or a functional variant thereof; an Escherichia coli homoserine dehydrogenase polypeptide or a functional variant thereof; a Corynebacterium glutamicum homoserine dehydrogenase polypeptide or a functional variant thereof; and an Erwinia chrysanthemi homoserine dehydrogenase polypeptide or a functional variant thereof. In certain embodiments the heterologous homoserine dehydrogenase polypeptide or functional variant thereof has reduced feedback inhibition.

In various embodiments the heterologous bacterial O-homoserine acetyltransferase polypeptide is chosen from: a Mycobacterium smegmatis O-homoserine acetyltransferase polypeptide or functional variant thereof; a Streptomyces coelicolor O-homoserine acetyltransferase polypeptide or a functional variant thereof; a Thermobifida fusca O-homoserine acetyltransferase polypeptide or a functional variant thereof; an Erwinia chrysanthemi O-homoserine acetyltransferase polypeptide or a functional variant thereof; an Escherichia coli O-homoserine acetyltransferase polypeptide or a functional variant thereof; and a Corynebacterium glutamicum O-homoserine acetyltransferase polypeptide or a functional variant thereof. In certain embodiments the heterologous O-homoserine acetyltransferase polypeptide or functional variant thereof has reduced feedback inhibition.

In various embodiments the heterologous bacterial O-acetylhomoserine sulfhydrylase polypeptide is chosen from: a Mycobacterium smegmatis O-acetylhomoserine sulfhydrylase or functional variant thereof; a Streptomyces coelicolor O-acetylhomoserine sulhydrylase polypeptide or a functional variant thereof; a Thermobifida fusca O-acetylhomoserine sulfhydrylase polypeptide or a functional variant thereof; and a Corynebacterium glutamicum O-acetylhomoserine sulfhydrylase polypeptide or a functional variant thereof. In certain embodiments the heterologous O-acetylhomoserine sulfhydrylase polypeptide or functional variant thereof has reduced feedback inhibition.

In various embodiments the bacterium further comprises a nucleic acid molecule encoding a heterologous bacterial methionine adenosyltransferase polypeptide (e.g., a Mycobacterium smegmatis methionine adenosyltransferase polypeptide or functional variant thereof; a Streptomyces coelicolor methionine adenosyltransferase polypeptide or a functional variant thereof; a Thermobifida fusca methionine adenosyltransferase polypeptide or a functional variant thereof; an Erwinia chrysanthemi methionine adenosyltransferase polypeptide or a functional variant thereof; an Escherichia coli methionine adenosyltransferase polypeptide or a functional variant thereof; or a Corynebacterium glutamicum methionine adenosyltransferase polypeptide or a functional variant thereof).

The invention features a coryneform bacterium or a bacterium of the family Enterobacteriaceae such as an Escherichia coli bacterium comprising at least two of: (a) a nucleic acid molecule encoding a heterologous bacterial homoserine dehydrogenase polypeptide or a functional variant thereof; (b) a nucleic acid molecule encoding a heterologous bacterial O-homoserine acetyltransferase polypeptide or a functional variant thereof; and (c) a nucleic acid molecule encoding a heterologous bacterial O-acetylhomoserine sulfhydrylase polypeptide or a functional variant thereof. In certain embodiments one or more of the heterologous bacterial polypetides or functional variants thereof has reduced feedback inhibition

In another aspect, the invention features an Escherichia coli or coryneform bacterium comprising at least one or two of: (a) a genetically altered nucleic acid molecule comprising a sequence encoding a bacterial aspartokinase polypeptide or a functional variant thereof; (b) a genetically altered nucleic acid molecule comprising a sequence encoding a bacterial aspartate semialdehyde dehydrogenase polypeptide or a functional variant thereof; (c) a genetically altered nucleic acid molecule comprising a sequence encoding a bacterial phosphoenolpyruvate carboxylase polypeptide or a functional variant thereof; and (d) a genetically altered nucleic acid molecule comprising a sequence encoding a bacterial dihydrodipicolinate synthase polypeptide or a functional variant thereof. In various embodiments, the genetically altered nucleic acid molecule is a genomic nucleic acid molecule (e.g., a genomic nucleic acid molecule in which a mutation has been introduced, e.g., into a coding or regulatory region of a gene). In various embodiments, the nucleic acid molecule is a recombinant nucleic acid molecule.

In various embodiments, at least one of the at least two genetically altered nucleic acid molecules encodes a heterologous polypeptide. In one embodiment, the bacterium comprises (a) and (b), (a) and (c), (a) and (d), (b) and (c), (b) and (d), or (c) and (d). In one embodiment,the bacterium comprises at least three of (a)-(e). In one embodiment, the bacterium has reduced activity of one or more of the following polypeptides, relative to a control: (a) a homoserine dehydrogenase polypeptide; (b) a homoserine kinase polypeptide; and (c) a phosphoenolpyruvate carboxykinase polypeptide. In one embodiment, the bacterium comprises a mutation in an endogenous hom gene or an endogenous thrB gene (e.g., a mutation that reduces activity of the polypeptide encoded by the gene (e.g., a mutation in a catalytic region) or a mutation that reduces expression of the polypeptide encoded by the gene (e.g., the mutation causes premature termination of the polypeptide), or a mutation which decreases transcript or protein stability or half life. In one embodiment, the bacterium comprises a mutation in an endogenous hom gene and an endogeous thrB gene. In one embodiment,the bacterium comprises a mutation in an endogenous pck gene.

In another aspect, the invention features an Escherichia coli or coryneform bacterium comprising at least one or two of: (a) a genetically altered nucleic acid molecule comprising a sequence encoding a bacterial phosphoenolpyruvate carboxylase polypeptide or a functional variant thereof; (b) a genetically altered nucleic acid molecule comprising a sequence encoding a bacterial aspartokinase polypeptide or a functional variant thereof: (c) a genetically altered nucleic acid molecule comprising a sequence encoding a bacterial aspartate semialdehyde dehydrogenase polypeptide or a functional variant thereof; (d) a genetically altered nucleic acid molecule comprising a sequence encoding a bacterial homoserine dehydrogenase polypeptide or a functional variant thereof; (e) a genetically altered nucleic acid molecule comprising a sequence encoding a bacterial homoserine O-acetyltransferase polypeptide or a functional variant thereof; (f) a genetically altered nucleic acid molecule comprising a sequence encoding a bacterial O-acetylhomoserine sulfhydrylase polypeptide or a functional variant thereof; (g) a genetically altered nucleic acid molecule comprising a sequence encoding a bacterial 5-methyltetrahydrofolate homocysteine methyltransferase polypeptide or a functional variant thereof; (h) a genetically altered nucleic acid molecule comprising a sequence encoding a bacterial O-succinylhomoserine (thio)-lyase polypeptide or a functional variant thereof; (i) a genetically altered nucleic acid molecule comprising a sequence encoding a bacterial 5-methyltetrahydropteroyltriglutamate-homocysteine methyltransferase polypeptide or a functional variant thereof; (j) a genetically altered nucleic acid molecule comprising a sequence encoding a bacterial methionine adenosyltransferase polypeptide or a functional variant thereof; (k) a genetically altered nucleic acid molecule comprising a sequence encoding a bacterial serine hydroxylmethyltransferase polypeptide or a functional variant thereof; and (l) a genetically altered nucleic acid molecule comprising a sequence encoding a bacterial cystathionine beta-lyase polypeptide or a functional variant thereof.

In various embodiments, at least one of the at least two genetically altered nucleic acid molecules encodes a heterologous polypeptide. In various embodiments, the bacterium comprises (a) and at least one of (b), (c), (d), (e), (f), (g), (h), (i), (j), (k), and (1). In various embodiments, the bacterium comprises (b) and at least one of (c), (d), (e), (f), (g), (h), (i), (j), (k), and (1). In various embodiments, the bacterium comprises (c) and at least one of (d), (e), (f), (g), (h), (i), (j), (k), and (1). In various embodiments, the bacterium comprises (d) and at least one of (e), (f), (g), (h), (i), (j), (k), and (1). In various embodiments, the bacterium comprises (e) and at least one of (f), (g), (h), (i), (j), (k), and (l). In various embodiments, the bacterium comprises (f) and at least one of (g), (h), (i), (j), (k), and (l). In various embodiments, the bacterium comprises (g) and at least one of (h), (i), (j), (k), and (l). In various embodiments, the bacterium comprises (h) and at least one of (i), (j), (k), and (l). In various embodiments, the bacterium comprises (i) and at least one of (j) (k), and (l). In various embodiments, the bacterium comprises (j) and at least one of (k), and (l). In various embodiments, the bacterium comprises (k) and (l). In various embodiments,the bacterium comprises at least three of (a)-(l).

In some embodiments, the bacterium has reduced activity of one or more of the following polypeptides, relative to a control: (a) a homoserine kinase polypeptide; (b) a phosphoenolpyruvate carboxykinase polypeptide; (c) a homoserine dehydrogenase polypeptide; and (d) a mcbR gene product polypeptide, e.g., the bacterium comprises a mutation in an endogenous hom gene, an endogenous thrB gene, an endogenous pck gene, or an endogenous mcbR gene, or combinations thereof.

In another aspect, the invention features an Escherichia coli or coryneform bacterium comprising at least two of: (a) a genetically altered nucleic acid molecule comprising a sequence encoding a bacterial phosphoenolpyruvate carboxylase polypeptide or a functional variant thereof; (b) a genetically altered nucleic acid molecule comprising a sequence encoding a bacterial aspartokinase polypeptide or a functional variant thereof; (c) a genetically altered nucleic acid molecule comprising a sequence encoding a bacterial aspartate semialdehyde dehydrogenase polypeptide or a functional variant thereof (d) a genetically altered nucleic acid molecule comprising a sequence encoding a bacterial homoserine dehydrogenase polypeptide or a functional variant thereof.

In various embodiments, at least one of the at least two polypeptides encodes a heterologous polypeptide.

In various embodiments, the bacterium comprises (a) and (b), (a) and (c), (a) and (d), (b) and (c), (b) and (d), or (c) and (d); or the bacterium comprises at least three of (a)-(d).

In various embodiments, the bacterium has reduced activity of one or more of the following polypeptides, relative to a control: (a) a phosphoenolpyruvate carboxykinase polypeptide; and (b) a mcbR gene product polypeptide, e.g., the bacterium comprises a mutation in an endogenous pck gene or an endogenous mcbR gene, e.g.,the bacterium comprises a mutation in an endogenous pck gene and an endogenous mcbR gene.

The invention also features a method of producing an amino acid or a related metabolite, the method comprising: cultivating a bacterium (e.g., a bacterium described herein) according to under conditions that allow the amino acid the metabolite to be produced, and collecting a composition that comprises the amino acid or related metabolite from the culture. The method can further include fractionating at least a portion of the culture to obtain a fraction enriched in the amino acid or the metabolite.

The invention also features a method for producing L-lysine, the method comprising: cultivating a bacterium described herein under conditions that allow L-lysine to be produced, and collecting the culture. The culture can be fractionated (e.g., to remove cells and/or to obtain fractions enriched in L-lysine).

In another aspect, the invention features a method for the preparation of animal feed additives comprising an aspartate-derived amino acid(s), the method comprising two or more of the following steps:
(a) cultivating a bacterium (e.g., a bacterium described herein) under conditions that allow the aspartate-derived amino acid(s) to be produced;
(b) collecting a composition that comprises at least a portion of the aspartate-derived amino acid(s);
(c) concentrating of the collected composition to enrich for the aspartate-derived amino acid(s); and
(d) optionally, adding of one or more substances to obtain the desired animal feed additive.

The substances that can be added include, e.g., conventional organic or inorganic auxiliary substances or carriers, such as gelatin, cellulose derivatives (e.g., cellulose ethers), silicas, silicates, stearates, grits, brans, meals, starches, gums, alginates sugars or others, and/or mixed and stabilized with conventional thickeners or binders.

In various embodiments, the composition that is collected lacks bacterial cells. In various embodiments, the composition that is collected contains less than 10%, 5%, 1%, 0.5% of the bacterial cells that result from cultivating the bacterium. In various embodiments, the composition comprises at least 1% (e.g., at least 1%, 5%, 10%, 20%, 40%, 50%, 75%, 80%, 90%, 95%, or to 100%) of that bacterial cells that result from cultivating the bacterium.

The invention features a method for producing L-methionine, the method comprising: cultivating a bacterium described herein under conditions that allow L-methionine to be produced, and collecting the culture. The culture can be fractionated (e.g., to remove cells and/or to obtain fractions enriched in L-methionine).

The invention features a method for producing S-adenosyl-L-methionine (S-AM), the method comprising: cultivating a bacterium described herein under conditions that allow S-adenosyl-L-methionine to be produced, and collecting the culture. The culture can be fractionated (e.g., to remove cells and/or to obtain fractions enriched in S-AM). The invention features a method for producing L-threonine or L-isoleucine, the method comprising: cultivating a bacterium described herein under conditions that allow L-threonine or L-isoleucine to be produced, and collecting the culture. The culture can be fractionated (e.g., to remove cells and/or to obtain fractions enriched in L-threonine or L-isoleucine). The invention also features methods for producing homoserine, O-acetylhomoserine, and derivatives thereof, the method comprising: cultivating a bacterium described herein under conditions that allow homoserine, O-acetylhomoserine, or derivatives thereof to be produced, and collecting the culture. The culture can be fractionated (e.g., to remove cells and/or to obtain fractions enriched in homoserine, O-acetylhomoserine, or derivatives thereof).

The invention features a coryneform bacterium or a bacterium of the family Enterobacteriaceae such as an Escherichia coli bacterium comprising a nucleic acid molecule that encodes a heterologous bacterial cystathionine beta-lyase polypeptide (e.g., a Mycobacterium smegmatis cystathionine beta-lyase polypeptide or functional variant thereof; a Bifidobacterium longum cystathionine beta-lyase polypeptide or a functional variant thereof; a Lactobacillus plantarum cystathionine beta-lyase polypeptide or a functional variant thereof; a Corynebacterium glutamicum cystathionine beta-lyase polypeptide or a functional variant thereof; an Escherichia coli cystathionine beta-lyase polypeptide or a functional variant thereof) or a functional variant thereof.

In various embodiments the Mycobacterium smegmatis cystathionine beta-lyase polypeptide comprises a sequence at least 80% identical to SEQ ID NO:59 (e.g., a sequence at 25 least 80%, 85%, 90%, 92%, 94%, 95%, 96%, 97%, 98%, 99% or more identical to SEQ ID NO:59), or a variant sequence thereof; the Bifidobacterium longum cystathionine beta-lyase polypeptide comprises SEQ ID NO:60 or a variant sequence thereof; the Lactobacillus plantarum cystathionine beta-lyase polypeptide comprises SEQ ID NO:61 or a variant sequence thereof; the Corynebacterium glutamicum cystathionine beta-lyase polypeptide comprises SEQ ID NO:217 or a variant sequence thereof; and the Escherichia coli cystathionine beta-lyase polypeptide comprises SEQ ID NO:218 or a variant sequence thereof.

The invention features a coryneform bacterium or a bacterium of the family Enterobacteriaceae such as an Escherichia coli bacterium comprising a nucleic acid molecule that encodes a heterologous bacterial glutamate dehydrogenase polypeptide (e.g., a Streptomyces coelicolor glutamate dehydrogenase or functional variant thereof; a Thermobifida fusca glutamate dehydrogenase polypeptide or a functional variant thereof; a Lactobacillus plantarum glutamate dehydrogenase polypeptide or a functional variant thereof; a Corynebacterium glutamicum glutamate dehydrogenase polypeptide or a functional variant thereof; a Escherichia coli glutamate dehydrogenase polypeptide or a functional variant thereof) or a functional variant thereof.

In various embodiments the Mycobacterium smegmatis glutamate dehydrogenase polypeptide comprises SEQ ID NO:62 or a variant sequence thereof; the Thermobifida fusca glutamate dehydrogenase polypeptide comprises SEQ ID NO:63 or a variant sequence thereof; the Lactobacillus plantarum glutamate dehydrogenase polypeptide comprises SEQ ID NO:65 or a variant sequence thereof; the Corynebacterium glutamicum glutamate dehydrogenase polypeptide comprises SEQ ID NO:219 or a variant sequence thereof; and the Escherichia coli glutamate dehydrogenase polypeptide comprises SEQ ID NO:220 or a variant sequence thereof.

The invention also features a coryneform bacterium or a bacterium of the family Enterobacteriaceae such as an Escherichia coli bacterium comprising a nucleic acid molecule that encodes a heterologous bacterial diaminopimelate dehydrogenase polypeptide or a functional variant thereof (e.g., a Bacillus sphaericus diaminopimelate dehydrogenase polypeptide or a functional variant thereof; a Corynebacterium glutamicum glutamate dehydrogenase polypeptide or a functional variant thereof).

In various embodiments the Bacillus sphaericus diaminopimelate dehydrogenase polypeptide comprises SEQ ID NO:65 or a variant sequence thereof.

The invention also features a coryneform bacterium or a bacterium of the family Enterobacteriaceae such as an Escherichia coli bacterium comprising a nucleic acid molecule that encodes a heterologous bacterial detergent sensitivity rescuer polypeptide (e.g., a Mycobacterium smegmatis detergent sensitivity rescuer polypeptide or functional variant thereof; a Streptomyces coelicolor detergent sensitivity rescuer polypeptide or a functional variant thereof; a Thermobifida fusca detergent sensitivity rescuer polypeptide or a functional variant thereof; a Corynebacterium glutamicum detergent sensitivity rescuer polypeptide or a functional variant thereof) or a functional variant thereof.

In various embodiments the Mycobacterium smegmatis detergent sensitivity rescuer polypeptide comprises a sequence at least 80% identical to either SEQ ID NO:68, SEQ ID NO:69 (e.g., a sequence at least 80%, 85%, 90%, 92%, 94%, 95%, 96%, 97%, 98more identical), or a variant sequence thereof; the heterologous bacterial detergent sensitivity rescuer polypeptide is a Streptomyces coelicolor detergent sensitivity rescuer polypeptide or functional variant thereof; the Streptomyces coelicolor detergent sensitivity rescuer polypeptide comprises SEQ ID NO:67 or a variant sequence thereof; the Thermobifida fusca detergent sensitivity rescuer polypeptide comprises SEQ ID NO:66 or a variant sequence thereof; and the Corynebacterium glutamicum detergent sensitivity rescuer polypeptide comprises SEQ ID NO:221 or a variant sequence thereof.The invention features a coryneform bacterium or a bacterium of the family Enterobacteriaceae such as an Escherichia coli bacterium comprising a nucleic acid molecule that encodes a heterologous bacterial 5-methyltetrahydrofolate homocysteine methyltransferase polypeptide (e.g., a Mycobacterium smegmatis 5-methyltetrahydrofolate homocysteine methyltransferase polypeptide or functional variant thereof; a Streptomyces coelicolor 5-methyltetrahydrofolate homocysteine methyltransferase polypeptide or a functional variant thereof; a Thermobifida fusca 5-methyltetrahydrofolate homocysteine methyltransferase polypeptide or a functional variant thereof; a Lactobacillus plantarum 5-methyltetrahydrofolate homocysteine methyltransferase polypeptide or a functional variant thereof; a Corynebacterium glutamicum 5-methyltetrahydrofolate homocysteine methyltransferase polypeptide or a functional variant thereof; a Escherichia coli 5-methyltetrahydrofolate homocysteine methyltransferase polypeptide or a functional variant thereof) or a functional variant thereof.

In various embodiments the Mycobacterium smegmatis 5-methyltetrahydrofolate homocysteine methyltransferase polypeptide comprises a sequence at least 80% identical to SEQ ID NO:72, SEQ ID NO:73 (e.g., a sequence at least 80%, 85%, 90%, 92%, 94%, 95%, 96%, 97%, 98%, 99% or more identical), or a variant sequence thereof; the Streptomyces coelicolor 5-methyltetrahydrofolate homocysteine methyltransferase polypeptide comprises SEQ ID NO:71 or a variant sequence thereof; the Thermobifida fusca 5-methyltetrahydrofolate homocysteine methyltransferase polypeptide comprises SEQ ID NO:70 or a variant sequence thereof; the Lactobacillus plantarum 5 -methyltetrahydrofolate homocysteine methyltransferase polypeptide comprises SEQ ID NO:74 or a variant sequence thereof; the Corynebacterium glutamicum 5-methyltetrahydrofolate homocysteine methyltransferase polypeptide comprises SEQ ID NO: 222 or a variant sequence thereof; and the Escherichia coli 5-methyltetrahydrofolate homocysteine methyltransferase polypeptide comprises SEQ ID NO:223 or a variant sequence thereof The invention also features a coryneform bacterium or a bacterium of the family Enterobacteriaceae such as an Escherichia coli bacterium comprising a nucleic acid molecule that encodes a heterologous bacterial 5-methyltetrahydropteroyltriglutamate-homocysteine methyltransferase polypeptide (e.g., a Mycobacterium smegmatis 5-methyltetrahydropteroyltriglutamate-homocysteine methyltransferase polypeptide or functional variant thereof; a Streptomyces coelicolor 5-methyltetrahydropteroyltriglutamate-homocysteine methyltransferase polypeptide or functional variant thereof; a Corynebacterium glutamicum 5-methyltetrahydropteroyltriglutamate-homocysteine methyltransferase polypeptide or a functional variant thereof; an Escherichia coli 5-methyltetrahydropteroyltriglutamate-homocysteine methyltransferase polypeptide or a functional variant thereof) or a functional variant thereof.

In various embodiments the Mycobacterium smegmatis 5-methyltetrahydropteroyltriglutamate-homocysteine methyltransferase polypeptide is at least 80% identical to SEQ ID NO:75 or SEQ ID NO:76 (e.g., a sequence at least 80%, 85%, 90%, 92%, 94%, 95%, 96%, 97%, 98%, 99% or more identical to SEQ ID NO:75 or SEQ ID NO:76); the Streptomyces coelicolor 5-methyltetrahydropteroyltriglutamate-homocysteine methyltransferase polypeptide comprises SEQ ID NO:77 or a variant sequence thereof; the Corynebacterium glutamicum 5-methyltetrahydropteroyltriglutamate-homocysteine methyltransferase polypeptide comprises SEQ ID NO:224 or a variant sequence thereof; and the Escherichia coli 5-methyltetrahydropteroyltriglutamate-homocysteine methyltransferase polypeptide comprises SEQ ID NO:225 or a variant sequence thereof.

The invention features a coryneform bacterium or a bacterium of the family Enterobacteriaceae such as an Escherichia coli bacterium comprising a nucleic acid molecule that encodes a heterologous bacterial serine hydroxymethyltransferas polypeptide (e.g., a Mycobacterium smegmatis serine hydroxymethyltransferase polypeptide or functional variant thereof; a Streptomyces coelicolor serine hydroxymethyltransferase polypeptide or a functional variant thereof; a Thermobifida fusca serine hydroxymethyltransferase polypeptide or a functional variant thereof; a Lactobacillus plantarum serine hydroxymethyltransferase polypeptide or a functional variant thereof; a Corynebacterium glutamicum serine hydroxymethyltransferase polypeptide or a functional variant thereof; an Escherichia coli serine hydroxymethyltransferase polypeptide or a functional variant thereof) or a functional variant thereof.

In various embodiments the Mycobacterium smegmatis serine hydroxymethyltransferase polypeptide is at least 80% identical to SEQ ID NO:80 or SEQ ID NO:81 (e.g., a sequence at least 80%, 85%, 90%, 92%, 94%, 95%, 96%, 97%, 98%, 99% or more identical to SEQ ID NO:80 or SEQ ID NO:81); the Streptomyces coelicolor serine hydroxymethyltransferase polypeptide comprises SEQ ID NO:78 or a variant sequence thereof; the Thermobifida fusca serine hydroxymethyltransferase polypeptide comprises SEQ ID NO:79 or a variant sequence thereof; the Lactobacillus plantarum serine hydroxymethyltransferase polypeptide comprises SEQ ID NO:82 or a variant sequence thereof; the Corynebacterium glutamicum serine hydroxymethyltransferase polypeptide comprises SEQ ID NO:226 or a variant sequence thereof; and the Escherichia coli serine hydroxymethyltransferase polypeptide comprises SEQ ID NO:227 or a variant sequence thereof.

The invention features a coryneform bacterium or a bacterium of the family Enterobacteriaceae such as an Escherichia coli bacterium comprising a nucleic acid molecule that encodes a heterologous bacterial 5,10-methylenetetrahydrofolate reductase polypeptide (e.g., a Streptomyces coelicolor 5,1 0-methylenetetrahydrofolate reductase polypeptide or a functional variant thereof; a Thermobifida fusca 5,10-methylenetetrahydrofolate reductase polypeptide or a functional variant thereof; a Corynebacterium glutamicum 5,1 0-methylenetetrahydrofolate reductase polypeptide or a functional variant thereof; an Escherichia coli 5,10-methylenetetrahydrofolate reductase polypeptide or a functional variant thereof) or a functional variant thereof.

In various embodiments the Streptomyces coelicolor 5,1 0-methylenetetrahydrofolate reductase polypeptide comprises SEQ ID NO:84 or a variant sequence thereof; the Thermobifida fusca 5,10-methylenetetrahydrofolate reductase polypeptide comprises SEQ ID NO: 83 or a variant sequence thereof; the Corynebacterium glutamicum 5,10-methylenetetrahydrofolate reductase polypeptide comprises SEQ ID NO: 228 or a variant sequence thereof; and the Escherichia coli 5,10-methylenetetrahydrofolate reductase polypeptide comprises SEQ ID NO: 229 or a variant sequence thereof.

The invention features a coryneform bacterium or a bacterium of the family Enterobacteriaceae such as an Escherichia coli bacterium comprising a nucleic acid molecule that encodes a heterologous bacterial serine O-acetyltransferase polypeptide (e.g., a Mycobacterium smegmatis serine O-acetyltransferase polypeptide or functional variant thereof; a Lactobacillus plantarum serine O-acetyltransferase polypeptide or a functional variant thereof; a Corynebacterium glutamicum serine O-acetyltransferase polypeptide or a functional variant thereof; an Escherichia coli serine O-acetyltransferase polypeptide or a functional variant thereof) or a functional variant thereof.

In various embodiments the Mycobacterium smegmatis serine O-acetyltransferase polypeptide is at least 80% identical to SEQ ID NO:85 or SEQ ID NO:86 (e.g., a sequence at least 80%, 85%, 90%, 92%, 94%, 95%, 96%, 97%, 98%, 99% or more identical to SEQ ID NO:85 or SEQ ID NO:86); the Lactobacillus plantarum serine O-acetyltransferase polypeptide comprises SEQ ID NO:87 or a variant sequence thereof; the Corynebacterium glutamicum serine O-acetyltransferase polypeptide comprises SEQ ID NO:230 or a variant sequence thereof; and the Escherichia coli serine O-acetyltransferase polypeptide comprises SEQ ID NO:231 or a variant sequence thereof.

The invention features a coryneform bacterium or a bacterium of the family Enterobacteriaceae such as an Escherichia coli bacterium comprising a nucleic acid molecule that encodes a heterologous bacterial D-3-phosphoglycerate dehydrogenase polypeptide (e.g., a Mycobacterium smegmatis D-3-phosphoglycerate dehydrogenase polypeptide or functional variant thereof; a Streptomyces coelicolor D-3-phosphoglycerate dehydrogenase polypeptide or a functional variant thereof; a Thermobifida fusca D-3-phosphoglycerate dehydrogenase polypeptide or a functional variant thereof; a Lactobacillus plantarum D-3-phosphoglycerate dehydrogenase polypeptide or a functional variant thereof; a Corynebacterium glutamicum D-3-phosphoglycerate dehydrogenase polypeptide or a functional variant thereof; an Escherichia coli D-3-phosphoglycerate dehydrogenase polypeptide or a functional vaant thereof) or a functional variant thereof.

In various embodiments the Mycobacterium smegmatis D-3-phosphoglycerate dehydrogenase polypeptide is at least 80% identical to SEQ ID NO:88 or SEQ ID NO:89 (e.g., a sequence at least 80%, 85%, 90%, 92%, 94%, 95%, 96%, 97%, 98%, 99% or more identical to SEQ ID NO:88 or SEQ ID NO:89); the Streptomyces coelicolor D-3-phosphoglycerate dehydrogenase polypeptide comprises SEQ ID NO:91 or a variant sequence thereof; the Thermobifida fusca D-3-phosphoglycerate dehydrogenase polypeptide comprises SEQ ID NO:90 or a variant sequence thereof; the Lactobacillus plantarum D-3-phosphoglycerate dehydrogenase polypeptide comprises SEQ ID NO:92 or a variant sequence thereof; the Corynebacterium glutamicum serine O-acetyltransferase polypeptide comprises SEQ ID NO:232 or a variant sequence thereof; and the Escherichia coli serine O-acetyltransferase polypeptide comprises SEQ ID NO:233 or a variant sequence thereof.

The invention features a coryneform bacterium or a bacterium of the family Enterobacteriaceae such as an Escherichia coli bacterium comprising a nucleic acid molecule that encodes a heterologous bacterial lysine exporter polypeptide (e.g., a Corynebacterium glutamicum lysine exporter polypeptide or functional variant thereof; a Mycobacterium smegmatis lysine exporter polypeptide or functional variant thereof; a Streptomyces coelicolor lysine exporter polypeptide or a functional variant thereof; an Escherichia coli lysine exporter polypeptide or functional variant thereof or a Lactobacillus plantarum lysine exporter protein or a functional variant thereof) or functional variant thereof.

In various embodiments the Mycobacterium smegmatis lysine exporter polypeptide is at least 80% identical to SEQ ID NO:93 or SEQ ID NO:94 (e.g., a sequence at least 80%, 85%, 90%, 92%, 94%, 95%, 96%, 97%, 98%, 99% or more identical to SEQ ID NO:93 or SEQ ID NO:94); the Streptomyces coelicolor lysine exporter polypeptide comprises SEQ ID NO:95 or a variant sequence thereof; the Lactobacillus plantarum lysine exporter polypeptide comprises SEQ ID NO:96 or a variant sequence thereof; the Corynebacterium glutamicum lysine exporter polypeptide comprises SEQ ID NO:234 or a variant sequence thereof; and the Escherichia coli lysine exporter polypeptide comprises SEQ ID NO:237 or a variant sequence thereof.

The invention features a coryneform bacterium or a bacterium of the family Enterobacteriaceae such as an Escherichia coli bacterium comprising a nucleic acid molecule that encodes a bacterial O-succinylhomoserine (thio)-lyase/O-acetylhomoserine (thio)-lyase polypeptide (e.g., a Corynebacterium glutamicum O-succinylhomoserine (thio)-lyase polypeptide or functional variant thereof; a Mycobacterium smegmatis O-succinylhomoserine (thio)-lyase polypeptide or functional variant thereof; a Streptomyces coelicolor O-succinylhomoserine (thio)-lyase polypeptide or a functional variant thereof; a Thermobifida fusca O-succinylhomoserine (thio)-lyase polypeptide or a functional variant thereof; an Escherichia coli O-succinylhomoserine (thio)-lyase polypeptide or a functional variant thereof; or a Lactobacillus plantarum O-succinylhomoserine (thio)-lyase polyp eptide or a functional variant thereof) or a functional variant thereof.

In various embodiments the Mycobacterium smegmatis O-succinylhomoserine (thio)-lyase polypeptide is at least 80% identical to SEQ ID NO:97 or SEQ ID NO:98 (e.g., a sequence at least 80%, 85%, 90%, 92%, 94%, 95%, 96%, 97%, 98%, 99% or more identical to SEQ ID NO:97 or SEQ ID NO:98); the Streptomyces coelicolor O-succinylhomoserine (thio)-lyase polypeptide comprises SEQ ID NO:99 or a variant sequence thereof; the Thermobifida fusca O-succinylhomoserine (thio)-lyase polypeptide comprises SEQ ID NO:100 or a variant sequence thereof; the Lactobacillus plantarum O-succinylhomoserine (thio)-lyase polypeptide comprises SEQ ID NO: 101 or a variant sequence thereof; the Corynebacterium glutamicum O-succinylhomoserine (thio)-lyase polypeptide comprises SEQ ID NO:235 or a variant sequence thereof; and the Escherichia coli O-succinylhomoserine (thio)-lyase polypeptide comprises SEQ ID NO:236 or a variant sequence thereof.

The invention features a coryneform bacterium or a bacterium of the family Enterobacteriaceae such as an Escherichia coli bacterium comprising a nucleic acid molecule that encodes a threonine efflux polypeptide (e.g. a Corynebacterium glutamicum threonine efflux polypeptide or a functional variant thereof; a homolog of the Corynebacterium glutamicum threonine efflux polypeptide or a functional variant thereof; a Streptomyces coelicolor putative threonine efflux polypeptide or a functional variant thereof) or functional variant thereof.

In various embodiments the Corynebacterium glutamicum threonine efflux polypeptide comprises SEQ ID NO: 196 or a variant sequence thereof; the homolog of the Corynebacterium glutamicum threonine efflux polypeptide comprises a homolog of SEQ ID NO: 196 or a variant sequence thereof; and the Streptomyces coelicolor putative threonine efflux polypeptide comprises SEQ ID NO: 102 or a variant sequence thereof.

The invention also features a coryneform bacterium or a bacterium of the family Enterobacteriaceae such as an Escherichia coli bacterium comprising a nucleic acid molecule that encodes C. glutamicum hypothetical polypeptide (SEQ ID NO: 198), a bacterial homolog of C. glutamicum hypothetical polypeptide (SEQ ID NO: 198), (e.g., a Mycobacterium smegmatis hypothetical polypeptide or functional variant thereof; a Streptomyces coelicolor hypothetical polypeptide or a functional variant thereof; a Thermobifida fusca hypothetical polypeptide or a functional variant thereof; an Escherichia coli hypothetical polypeptide or a functional variant thereof; or a Lactobacillus plantarum hypothetical polypeptide or a functional variant thereof) or a functional variant thereof.

In various embodiments the the bacterial homolog is: a Mycobacterium smegmatis hypothetical polypeptide at least 80% identical to SEQ ID NO:104 or SEQ ID NO:105 (e.g., a sequence at least 80%, 85%, 90%, 92%, 94%, 95%, 96%, 97%, 98%, 99% or more identical to SEQ ID NO: 104 or SEQ ID NO: 105); the Streptomyces coelicolor hypothetical polypeptide comprises SEQ ID NO:103 or a variant sequence thereof; the Thermobifida fusca hypothetical polypeptide comprises SEQ ID NO106 or a variant sequence thereof; the Lactobacillus plantarum hypothetical polypeptide comprises SEQ ID NO:107 or a variant sequence thereof.

The invention also features a coryneform bacterium or a bacterium of the family Enterobacteriaceae such as an Escherichia coli bacterium comprising a nucleic acid molecule that encodes C. glutamicum putative membrane polypeptide (SEQ ID NO:201), a bacterial homolog of C. glutamicum putative membrane polypeptide (SEQ ID NO:201), (e.g., a Streptomyces coelicolor putative membrane polypeptide or a functional variant thereof; a Thermobifida fusca putative membrane polypeptide or a functional variant thereof; an Erwinia chrysanthemi putative membrane polypeptide or a functional variant thereof; an Escherichia coli putative membrane polypeptide or a functional variant thereof; a Lactobacillus plantarum putative membrane polypeptide or a functional variant thereof; or a Pectobacterium chrysanthemi putative membrane polypeptide or a functional variant thereof) or a functional variant thereof.

In various embodiments the Streptomyces coelicolor putative membrane polypeptide comprises SEQ ID NO:111, SEQ ID NO: 112, SEQ ID NO: 113, SEQ ID NO: 114, oravariant sequence thereof; the Thermobifida fusca putative membrane polypeptide comprises SEQ ID NO: 108, SEQ ID NO: 109, SEQ ID NO: 110, or a variant sequence thereof; the Erwinia chrysanthemi putative membrane polypeptide comprises SEQ ID NO: 115 or a variant sequence thereof; the Pectobacterium chrysanthemi putative membrane polypeptide comprises SEQ ID NO:116 or a variant sequence thereof; the Lactobacillus plantarum putative membrane polypeptide comprises SEQ ID NO:1 17, SEQ ID NO:1 18, SEQ ID NO:1 19, or a variant sequence thereof.

The invention also features a coryneform bacterium or a bacterium of the family Enterobacteriaceae such as an Escherichia coli bacterium comprising a nucleic acid molecule that encodes C. glutamicum drug permease polypeptide (SEQ ID NO:199), a bacterial homolog of C. glutamicum drug permease polypeptide (SEQ ID NO: 199), (e.g., a Streptomyces coelicolor drug permease polypeptide or a functional variant thereof; a Thermobifida fusca drug permease polypeptide or a functional variant thereof; an Escherichia coli drug permease polypeptide or a functional variant thereof;or a Lactobacillus plantarum drug permease polypeptide or a functional variant thereof) or a functional variant thereof.

In various embodiments the Streptomyces coelicolor drug permease polypeptide comprises SEQ ID NO: 120, SEQ ID NO: 121, or a variant sequence thereof; the Thermobifida fusca drug permease polypeptide comprises SEQ ID NO: 122, SEQ ID NO: 123, or a variant sequence thereof; the Lactobacillus plantarum drug permease polypeptide comprises SEQ ID NO: 124 or a variant sequence thereof.

The invention also features a coryneform bacterium or a bacterium of the family Enterobacteriaceae such as an Escherichia coli bacterium comprising a nucleic acid molecule that encodes C. glutamicum hypothetical membrane polypeptide (SEQ iID NO: 197), a bacterial homolog of C. glutamicum hypothetical membrane polypeptide (SEQ ID NO: 197), (e.g., a Thermobifida fusca hypothetical membrane polypeptide or a functional variant thereof).

In various embodiments the Thermobifida fusca hypothetical membrane polypeptide comprises SEQ ID NO:125 or a variant sequence thereof.

As mentioned above, the invention also provides nucleic acids encoding variant bacterial proteins. Nucleic acids that include sequences encoding variant bacterial polypeptides can be expressed in the organism from which the sequence was derived, or they can be expressed in an organism other than the organism from which they were derived (e.g., heterologous organisms).

In one aspect, the invention features an isolated nucleic acid (e.g., a nucleic acid expression vector) that encodes a variant of a bacterial polypeptide (e.g., a variant of a wild-type bacterial polypeptide) that regulates the production of one or more amino acids from the aspartic acid family of amino acids or related metabolites. The bacterial polypeptide can include, for example, the following amino acid sequence: G₁-X₂-K₃-X₄-X₅-X₆-X₇-X₈-X₉-X₁₀-X₁₁-X₁₂-X₁₃-X₁₃ₐ-X_{13b}-X_{13c}-X_{13d}-X₁₃ₑ-X_{13f}-X_{13g}-X₁₃ₕ-X₁₃ᵢ-X₁₃ⱼ-X₁₃ₖ-X₁₃ₗ-F₁₄-X₁₅-Z₁₆-X₁₇-X₁₈-X₁₉-X₂₀-X₂₁-X₂₁ₐ-X_{21b}-X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-X₂₁ₘ-X₂₁ₙ-X₂₁ₒ-X₂₁ₚ-X_{21q}-Xᵣ-X₂₁ₛ-X₂₁ₜ-D₂₂ (SEQ ID NO:360), wherein each of X₂, X₄-X₁₃, X₁₅, and X₁₇-X₂₀ is, independently, any amino acid, wherein each of X₁₃ₐ-X₁₃ₗ is, independently, any amino acid or absent, wherein each of X₂₁ₐ-X₂₁ₜ is, independently, any amino acid or absent, and wherein Z₁₆ is selected from valine, aspartate, glycine, isoleucine, and leucine. The variant of the bacterial polypeptide includes an amino acid change relative to the bacterial protein, e.g., at one or more of G₁, K₃, F₁₄, Z₁₆, or D₂₂ of SEQ ID NO:360, or at an amino acid within 8, 5, 3, 2, or 1 residue of G₁, K₃, F₁₄, Z₁₆, or D₂₂ of SEQ ID NO:360. In one embodiment, variant of the bacterial polypeptide is otherwise identical in amino acid sequence to the bacterial protein, or at least 80%, 85%, 90%, 92%, 94%, 95%, 96%, 97%, 98%, 99%, or more identical to the bacterial polypeptide, e.g., the variant comprises fewer than 50, 40, 25, 15, 10, 7, 5, 3, 2, or 1 changes relative to the bacterial polypeptide.

Alternatively, or in addition, the bacterial polypeptide includes the following amino acid sequence: L₁-X₂-X₃-G₄-G₅-X₆-F₇-X₈-X₉-X₁₀-X₁₁ (SEQ ID NO:361), wherein each of X₂, X₄-X₁₃, X₁₅, and X₁₇-X₂₀ is, independently, any amino acid,wherein X₈ is selected from valine, leucine, isoleucine, and aspartate, and wherein X₁₁ is selected from valine, leucine, isoleucine, phenylalanine, and methionine; and the variant of the bacterial protein includes an amino acid change e.g., at one or more of L₁, G₄, X₈, X₁₁, or at an amino acid residue within 8, 5, 3, 2, or 1 residue of L₁, G₄, X₈, or X₁₁ of SEQ ID NO: 361).

In various embodiments, feedback inhibition of the variant of the bacterial polypeptide by S-adenosylmethionine is reduced, e.g., relative to the bacterial polypeptide (e.g., relative to a wild-type bacterial protein) or relative to a reference protein.

Amino acid changes in the variant of the bacterial polypeptide can be changes to alanine (e.g., wherein the original residue is other than an alanine) or non-conservative changes. The changes can be conservative changes.

The invention also features polypeptides encoded by the nucleic acids described herein, e.g., a polypeptide encoded by a nucleic acid that encodes a variant of a bacterial polypeptide (e.g., a variant of a wild-type bacterial polypeptide) that regulates the production of one or more amino acids from the aspartic acid family of amino acids or related metabolites, wherein the bacterial polypeptide includes SEQ ID NO:360 or SEQ ID NO:361, and wherein the variant includes an amino acid change relative to the bacterial polypeptide.

Also provided is a method for making a nucleic acid encoding a variant of a bacterial polypeptide that regulates the production of one or more amino acids from the aspartic acid family of amino acids or related metabolites. The method includes, for example, identifying a motif in the amino acid sequence of a wild-type form of the bacterial polypeptide, and constructing a nucleic acid that encodes a variant wherein one or more amino acid residues (e.g., one, two, three, four, or five residues) within and/or near (e.g., within 10, 8, 7, 5, 3, 2, or 1 residues) the motif is changed.

In various embodiments, the motif in the bacterial polypeptide includes the following amino acid sequence: G₁-X₂-K₃-X₄-X₅-X₆-X₇-X₈-X₉-X₁₀-X₁₁-X_{X}₁₂-X₁₃-X₁₃ₐ-X_{13b}-X_{13c}-X_{13d}-X₁₃ₑ-X_{13f}-X_{13g}-X₁₃ₕ-X₁₃ᵢ-X₁₃ⱼ-X₁₃ₖ-X₂₃ₗ-F₁₄-X₁₅-Z₁₆-X₁₇-X₁₈-X₁₉-X₂₀-X₂₁-X₂₁ₐ-X_{21b}-X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-X₂₁ₘ-X₂₁ₙ-X₂₁ₒ-X₂₁ₚ-X_{21q}-X₂₁ᵣ-X₂₁ₛ-X₂₁ₜ-D₂₂ (SEQ ID NO:360), wherein each of X₂, X₄-X₁₃, X₁₅, and X₁₇-X₂₀ is, independently, any amino acid, wherein each of X₁₃ₐ-X₁₃ₗ is, independently, any amino acid or absent, wherein each of X₂₁ₐ-X₂₁ₜ is, independently, any amino acid or absent, and wherein Z₁₆ is selected from valine, aspartate, glycine, isoleucine, and leucine. In various embodiments, one or more of G₁, K₃, F₁₄, Z₁₆, or D₂₂ of SEQ ID NO:360 is changed. In one embodiment, the variant of the bacterial polypeptide is otherwise identical in amino acid sequence to the bacterial polypeptide. In various embodiments, the motif in the bacterial polypeptide includes the following amino acid sequence: L₁-X₂-X₃-G₄-G₅-X₆-F₇-X₈-X₉- X₁₀-X₁₁ (SEQ ID NO:361), wherein each of X₂, X₄-X₁₃, X₁₅, and X₁₇-X₂₀ is, independently, any amino acid, wherein X₈ is selected from valine, leucine, isoleucine, and aspartate, and wherein X₁₁ is selected from valine, leucine, isoleucine, phenylalanine, and methionine. In various embodiments, one or more of L₁, G₄, X₈, X₁₁ of SEQ ID NO: 361 is changed. In one embodiment, the variant of the bacterial polypeptide is otherwise identical in amino acid sequence to the bacterial protein.

The invention also features a bacterium that includes a nucleic acid described herein, e.g., a nucleic acid that encodes a variant of a bacterial polypeptide (e.g., a variant of a wild-type bacterial polypeptide) that regulates the production of one or more amino acids from the aspartic acid family of amino acids or related metabolites, wherein the bacterial polypeptide includes SEQ ID NO:360 or SEQ ID NO:361, and wherein the variant includes an amino acid change relative to the bacterial polypeptide. The bacterium can be a genetically modified bacterium, e.g., a bacterium that has been modified to include the nucleic acid (e.g., by transformation of the nucleic acid, e.g., wherein the nucleic acid is episomal, or wherein the nucleic acid integrates into the genome of the bacterium, either at a random location, or at a specifically targeted location), and/or that has been modified within its genome (e.g., modified such that an endogenous gene has been altered by mutagenesis or replaced by recombination, or modified to include a heterologous promoter upstream of an endogenous gene.

The invention also features a method for producing an amino acid or a related metabolite. The methods can include, for example: cultivating a bacterium (e.g., a genetically modified bacterium) that includes a nucleic acid encoding a variant of a bacterial polypeptide (e.g., a variant of a wild-type bacterial polypeptide) that regulates the production of one or more amino acids from the aspartic acid family of amino acids or related metabolites, wherein the bacterial polypeptide includes SEQ ID NO:360 or SEQ ID NO:361, and wherein the variant includes an amino acid change relative to the bacterial polypeptide. The bacterium is cultivated under conditions in which the nucleic acid is expressed and that allow the amino acid (or related metabolite(s)) to be produced, and a composition that includes the amino acid (or related metabolite(s)) is collected. The composition can include, for example, culture supernatants, heat or otherwise killed cells, or purified amino acid.

In one aspect, the invention features an isolated nucleic acid encoding a variant bacterial homoserine O-acetyltransferase polypeptide. In certain embodiments, the variant bacterial homoserine O-acetyltransferase polypeptide exhibits reduced feedback inhibition, e.g., relative to a wild-type form of the bacterial homoserine O-acetyltransferase polypeptide. In various embodiments, the nucleic acid encodes a homoserine O-acetyltransferase polypeptide with reduced feedback inhibition by S-adenosylmethionine. In various embodiments, the bacterial homoserine O-acetyltransferase polypeptide is chosen from: a Corynebacterium glutamicum homoserine O-acetyltransferase polypeptide, a Mycobacterium smegmatis homoserine O-acetyltransferase polypeptide, a Thermobifida fusca homoserine O-acetyltransferase polypeptide, an Amycolatopsis mediterranei homoserine O-acetyltransferase polypeptide, a Streptomyces coelicolor homoserine O-acetyltransferase polypeptide, an Erwinia chrysanthemi homoserine O-acetyltransferase polypeptide, a Shewanella oneidensis homoserine O-acetyltransferase polypeptide, a Mycobacterium tuberculosis homoserine O-acetyltransferase polypeptide, an Escherichia coli homoserine O-acetyltransferase polypeptide, a Corynebacterium acetoglutamicum homoserine O-acetyltransferase polypeptide, a Corynebacterium melassecola homoserine O-acetyltransferase polypeptide, a Corynebacterium thermoaminogenes homoserine O-acetyltransferase polypeptide, a Brevibacterium lactofermentum homoserine O-acetyltransferase polypeptide, a Brevibacterium lactis homoserine O-acetyltransferase polypeptide, and a Brevibacterium flavum homoserine O-acetyltransferase polypeptide.

In another aspect, the invention features an isolated nucleic acid encoding a variant bacterial homoserine O-acetyltransferase polypeptide, wherein the variant homoserine O-acetyltransferase polypeptide is a variant of a homoserine O-acetyltransferase polypeptide including the following amino acid sequence: G₁-X₂-K₃-X₄-X₅-X₆-X₇-X₈-X₉-X₁₀-X₁₁-X₁₂-X₁₃-X₁₃ₐ-X_{13b}-X_{13c}-X_{13d}-X₁₃ₑ-X_{13f}-X_{13g}-X₁₃ₕ-X₁₃ᵢ-X₁₃ⱼ-X₁₃ₖ-X₁₃ₗ-F₁₄-X₁₅-Z₁₆-X₁₇-X₁₈-X₁₉-X₂₀-X₂₁-X₂₁ₐ-X_{21b}-X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-X₂₁ₘ-X₂₁ₙ-X₂₁ₒ-X₂₁ₚ-X_{21q}-X₂₁ᵣ-X₂₁ₛ-X₂₁ₜ-D₂₂ (SEQ ID NO:360), wherein each of X₂, X₄-X₁₃, X₁₅, and X₁₇-X₂₀ is, independently, any amino acid, wherein each of X₁₃ₐ-X₁₃ₗ is, independently, any amino acid or absent, wherein each of X₂₁ₐ-X₂₁ₜ is, independently, any amino acid or absent, and wherein Z₁₆ is selected from valine, aspartate, glycine, isoleucine, and leucine; wherein the variant homoserine O-acetyltransferase polypeptide includes an amino acid change at one or more of G₁, K₃, F₁₄, Z₁₆, or D₂₂ of SEQ ID NO:360. In various embodiments, the amino acid change is a change to an alanine.

In another aspect, the invention features an isolated nucleic acid encoding a variant bacterial homoserine O-acetyltransferase polypeptide, wherein the variant homoserine O-acetyltransferase polypeptide is a C. glutamicum homoserine O-acetyltransferase polypeptide including an amino acid change in one or more of the following residues of SEQ ID NO:212: Glycine 231, Lysine 233, Phenylalanine 251, Valine 253, and Aspartate 269. In various embodiments, the amino acid change is a change to an alanine.

In another aspect, the invention features an isolated nucleic acid encoding a variant bacterial homoserine O-acetyltransferase polypeptide, wherein the variant homoserine O-acetyltransferase polypeptide is a T fusca homoserine O-acetyltransferase polypeptide including an amino acid change in one or more of the following residues of SEQ ID NO:24: Glycine 81, Aspartate 287, Phenylalanine 269.

In another aspect, the invention features an isolated nucleic acid encoding a variant bacterial homoserine O-acetyltransferase polypeptide, wherein the variant homoserine O-acetyltransferase polypeptide is an E. coli homoserine O-acetyltransferase polypeptide including an amino acid change at Glutamate 252 of SEQ ID NO:213.

In another aspect, the invention features an isolated nucleic acid encoding a variant bacterial homoserine O-acetyltransferase polypeptide, wherein the variant homoserine O-acetyltransferase polypeptide is a mycobacterial homoserine O-acetyltransferase polypeptide including an amino acid change in a residue corresponding to one or more of the following residues of M leprae homoserine O-acetyltransferase polypeptide set forth in SEQ ID NO: 23: Glycine 73, Aspartate 278, and Tyrosine 260. In various embodiments, the variant bacterial homoserine O-acetyltransferase polypeptide is a variant of a M. smegmatis homoserine O-acetyltransferase polypeptide.

In another aspect, the invention features an isolated nucleic acid encoding a variant bacterial homoserine O-acetyltransferase polypeptide, wherein the variant homoserine O-acetyltransferase polypeptide is an M. tuberculosis homoserine O-acetyltransferase polypeptide including an amino acid change in one or more of the following residues of SEQ ID NO:22: Glycine 73, Tyrosine 260, and Aspartate 278.

The invention also features polypeptides encoded by, and bacteria including, the nucleic acids encoding variant bacterial homoserine O-acetyltransferases. In various embodiments, the bacteria are coryneform bacteria. The bacteria can further include nucleic acids encoding other variant bacterial proteins (e.g., variant bacterial proteins involved in amino acid production, e.g., variant bacterial proteins described herein).

In another aspect, the invention features a method for producing L-methionine or related intermediates such as O-acetyl homoserine, cystathionine, homocysteine, methionine, SAM and derivatives thereof, the method including: cultivating a genetically modified bacterium including a nucleic acid encoding a variant bacterial homoserine O-acetyltransferase under conditions in which the nucleic acid is expressed and that allow L-methionine (or related intermediate) to be produced, and collecting the culture. The culture can be fractionated (e.g., to remove cells and/or to obtain fractions enriched in L-methionine).

In another aspect, the invention features an isolated nucleic acid encoding a variant bacterial O-acetylhomoserine sulfhydrylase polypeptide. In certain embodiments, the variant bacterial homoserine O-acetylhomoserine sulfhydrylase polypeptide exhibits reduced feedback inhibition, e.g., relative to a wild-type form of the bacterial O-acetylhomoserine sulfhydrylase polypeptide.

In various embodiments, the nucleic acid encodes an O-acetylhomoserine sulfhydrylase polypeptide with reduced feedback inhibition by S-adenosylmethionine.

In various embodiments, the bacterial O-acetylhomoserine sulfhydrylase polypeptide is chosen from: a Corynebacterium glutamicum homoserine O-acetylhomoserine sulfhydrylase polypeptide, a Mycobacterium smegmatis homoserine O-acetylhomoserine sulfhydrylase polypeptide, a Thermobifida fusca O-acetylhomoserine sulfhydrylase polypeptide, an Amycolatopsis mediterranei O-acetylhomoserine sulfhydrylase polypeptide, a Streptomyces coelicolor O-acetylhomoserine sulfhydrylase polypeptide, an Erwinia chrysanthemi homoserine O-acetylhomoserine sulfhydrylase polypeptide, a Shewanella oneidensis O-acetylhomoserine sulfhydrylase polypeptide, a Mycobacterium tuberculosis O-acetylhomoserine sulfhydrylase polypeptide, an Escherichia coli O-acetylhomoserine sulfhydrylase polypeptide, a Corynebacterium acetoglutamicum O-acetylhomoserine sulfhydrylase polypeptide, a Corynebacterium melassecola O-acetylhomoserine sulfhydrylase polypeptide, a Corynebacterium thermoaminogenes O-acetylhomoserine sulfhydrylase polypeptide, a Brevibacterium lactofermentum O-acetylhomoserine sulfhydrylase polypeptide, a Brevibacterium lactis O-acetylhomoserine sulfhydrylase polypeptide, and a Brevibacterium flavum O-acetylhomoserine sulfhydrylase polypeptide.

In another aspect, the invention features an isolated nucleic acid encoding a variant bacterial O-acetylhomoserine sulfhydrylase polypeptide, wherein the variant O-acetylhomoserine sulfhydrylase polypeptide is a variant of an O-acetylhomoserine sulfhydrylase polypeptide including the following amino acid sequence: G₁-X₂-K₃-X₄-X₅-X₆-X₇-X₈-X₉-X₁₀-X₁₁-X₁₂-X₁₃-X₁₃ₐ-X_{13b}-X_{13c}-X_{13d}-X₁₃ₑ-X_{13f}-X_{13g}-X₁₃ₕ-X₁₃ᵢ-X₁₃ⱼ-X₁₃ₖ-X₁₃ₗ-F₁₄-X₁₅-Z₁₆-X₁₇-X₁₈-X₁₉-X₂₀-X₂₁-X₂₁ₐ-X_{21b}-X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-X₂₁ₘ-X₂₁ₙ-X₂₁ₒ-X₂₁ₚ-X_{21q}-X₂₁ᵣ-X₂₁ₛ-X₂₁ₜ-D₂₂ (SEQ ID NO:360), wherein each of X₂, X₄-X₁₃, X₁₅, and X₁₇-X₂₀ is, independently, any amino acid, wherein each of X₁₃ₐ-X₁₃ₗ is, independently, any amino acid or absent, wherein each of X₂₁ₐ-X₂₁ₜ is, independently, any amino acid or absent, and wherein Z₁₆ is selected from valine, aspartate, glycine, isoleucine, and leucine; wherein the variant O-acetylhomoserine sulfhydrylase polypeptide includes an amino acid change at one or more of G₁, K₃, F₁₄, Z₁₆, or D₂₂ of SEQ ID NO:360.

In various embodiments, the amino acid change is a change to an alanine.

In another aspect, the invention features an isolated nucleic acid encoding a variant bacterial O-acetylhomoserine sulfhydrylase polypeptide, wherein the variant O-acetylhomoserine sulfhydrylase polypeptide is a variant of a O-acetylhomoserine sulffiydrylase polypeptide including the following amino acid sequence: L₁-X₂-X₃-G₄-G₅-X₆-F₇-X₈-X₉-X₁₀-X₁₁ (SEQ ID NO:361), wherein X is any amino acid, wherein X₈ is selected from valine, leucine, isoleucine, and aspartate, and wherein X₁₁ is selected from valine, leucine, isoleucine, phenylalanine, and methionine; wherein the variant of the bacterial polypeptide includes an amino acid change at one or more of L₁, G₄, X₈, X₁₁ of SEQ ID NO:361.

In various embodiments, the amino acid change is a change to an alanine.

In another aspect, the invention features an isolated nucleic acid encoding a variant bacterial O-acetylhomoserine sulfhydrylase polypeptide, wherein the variant O-acetylhomoserine sulfhydrylase polypeptide is a C. glutamicum O-acetylhomoserine sufhydrylase polypeptide including an amino acid change in one or more of the following residues of SEQ ID NO:214: Glycine 227, Leucine 229, Aspartate 231, Glycine 232, Glycine 233, Phenylalanine 235, Aspartate 236, Valine 239, Phenylalanine 368, Aspartate 370, Aspartate 383, Glycine 346, and Lysine 348. In various embodiments, the amino acid change is a change to an alanine.

In another aspect, the invention features an isolated nucleic acid encoding a variant bacterial O-acetylhomoserine sulffiydrylase polypeptide, wherein the variant O-acetylhomoserine sulfhydrylase polypeptide is a T. fusca O-acetylhomoserine sulfhydrylase polypeptide including an amino acid change in one or more of the following residues of SEQ ID NO:25: Glycine 240, Aspartate 244, Phenylalanine 379, and Aspartate 394.

In another aspect, the invention features an isolated nucleic acid encoding a variant bacterial O-acetylhomoserine sulfhydrylase polypeptide, wherein the variant O-acetylhomoserine sulfhydrylase polypeptide is a M. smegmatis O-acetylhomoserine sulfhydrylase polypeptide including an amino acid change in one or more of the following residues of SEQ ID NO:287: Glycine 303, Aspartate 307, Phenylalanine 439, Aspartate 454.

In another aspect, the invention features a polypeptide encoded by a nucleic acid encoding a variant bacterial O-acetylhomoserine sulfhydrylase.

In another aspect, the invention features a bacterium comprising the nucleic acid encoding a variant bacterial O-acetylhomoserine sulfhydrylase polypeptide. In various embodiments, the bacterium is a coryneform bacterium. The bacterium can further comprise one or more nucleic acids encoding other variant bacterial polypeptides (e.g., variant bacterial polypeptides involved in amino acid production, e.g., a variant bacterial polypeptide described herein).

In another aspect, the invention features a method for producing L-methionine or related intermediates (e.g., homocysteine, methionine, S-AM, or derivatives thereof), the method comprising: cultivating a genetically modified bacterium comprising the nucleic acid encoding a variant bacterial O-acetylhomoserine sulfhydrylase polypeptide under conditions in which the nucleic acid is expressed and that allow L-methionine to be produced, and collecting the culture. The culture can be fractionated (e.g., to remove cells and/or to obtain fractions enriched in L-methionine).

In another aspect, the invention features an isolated nucleic acid encoding a variant bacterial mcbR gene product. In various embodiments, the variant bacterial mcbR gene product exhibits reduced feedback inhibition relative to a wild-type form of the mcbR gene product. In various embodiments, the nucleic acid encodes a mcbR gene product with reduced feedback inhibition by S-adenosylmethionine. In various embodiments, the bacterial mcbR gene product is chosen from: a Corynebacterium glutamicum mcbR gene product, a Corynebacterium acetoglutamicum mcbR gene product, a Corynebacterium melassecola mcbR gene product, and a Corynebacterium thermoaminogenes mcbR gene product.

In another aspect, the invention features an isolated nucleic acid encoding a variant bacterial mcbR gene product, wherein the variant mcbR gene product is a variant of an mcbR gene product including the following amino acid sequence: G₁-X₂-K₃-X₄-X₅-X₆-X₇-X₈-X₉-X₁₀-X₁₁-X₁₂-X₁₃-X₁₃ₐ-X_{13b}-X_{13c}-X_{13d}-X₁₃ₑ-X_{13f}-X_{13g}-X₁₃ₕ-X₁₃ᵢ-X₁₃ⱼ-X₁₃ₖ-X₁₃ₗ-F₁₄-X₁₅-Z₁₆-X₁₇-X₁₈-X₁₉-X₂₀-X₂₁-X₂₁ₐ-X_{21b}-X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-X₂₁ₘ-X₂₁ₙ-X₂₁ₒ-X₂₁ₚ-X_{21q}-X₂₁ᵣ-X₂₁ₛ-X₂₁ₜ-D₂₂ (SEQ ID NO:360), wherein each of X₂, X₄-X₁₃, X₁₅, and X₁₇-X₂₀ is, independently, any amino acid, wherein each of X₁₃ₐ-X₁₃ₗ is, independently, any amino acid or absent, wherein each of X₂₁ₐ-X₂₁ₜ is, independently, any amino acid or absent, and wherein Z₁₆ is selected from valine, aspartate, glycine, isoleucine, and leucine; wherein the variant mcbR gene product includes an amino acid change at one or more of G₁, K₃, F₁₄, Z₁₆, or D₂₂ of SEQ ID NO:360. In various embodiments, the amino acid change is a change to an alanine.

In another aspect, the invention features an isolated nucleic acid encoding a variant bacterial mcbR gene product, wherein the variant mcbR gene product is a C. glutamicum mcbR gene product including an amino acid change in one or more of the following residues of SEQ ID NO:363: Glycine 92, Lysine 94, Phenylalanine 116, Glycine 118, and Aspartate 134. In various embodiments, the amino acid change is a change to an alanine.

The invention also features a polypeptide encoded by the nucleic acids encoding a variant bacterial mcbR gene product.

The invention also features a bacterium including the nucleic acids encoding a variant bacterial mcbR gene product. In various embodiments, the bacterium is a coryneform bacterium. The bacterium can further comprise one or more nucleic acids encoding other variant bacterial polypeptides (e.g., variant bacterial polypeptides involved in amino acid production, e.g., variant bacterial polypeptides described herein).

The invention also features methods for producing L-methionine, the method including: cultivating a genetically modified bacterium including a nucleic acid encoding a variant bacterial mcbR gene product under conditions in which the nucleic acid is expressed and that allow L-methionine to be produced, and collecting the culture. The culture can be fractionated (e.g., to remove cells and/or to obtain fractions enriched in L-methionine).

In another aspect, the invention features an isolated nucleic acid encoding a variant bacterial aspartokinase polypeptide. In various embodiments, the variant bacterial aspartokinase polypeptide exhibits reduced feedback inhibition relative to a wild-type form of the bacterial aspartokinase polypeptide. In various embodiments, the nucleic acid encodes an aspartokinase polypeptide with reduced feedback inhibition by S-adenosylmethionine. In various embodiments, the bacterial aspartokinase polypeptide is chosen from: a Corynebacterium glutamicum aspartokinase polypeptide, a Mycobacterium smegmatis aspartokinase polypeptide, a Thermobifida fusca aspartokinase polypeptide, an Amycolatopsis mediterranei aspartokinase polypeptide, a Streptomyces coelicolor aspartokinase polypeptide, an Erwinia chrysanthemi aspartokinase polypeptide, a Shewanella oneidensis aspartokinase polypeptide, a Mycobacterium tuberculosis aspartokinase polypeptide, an Escherichia coli aspartokinase polypeptide, a Corynebacterium acetoglutamicum aspartokinase polypeptide, a Corynebacterium melassecola aspartokinase polypeptide, a Corynebacterium thermoaminogenes aspartokinase polypeptide, a Brevibacterium lactofermentum aspartokinase polypeptide, a Brevibacterium lactis aspartokinase polypeptide, and a Brevibacterium flavum aspartokinase polypeptide.

In another aspect, the invention features an isolated nucleic acid encoding a variant bacterial aspartokinase polypeptide, wherein the variant aspartokinase polypeptide is a variant of an aspartokinase polypeptide including the following amino acid sequence: G₁-X₂-K₃-X₄-X₅-X_{X}₆-X₇-X₈-X₉-X₁₀-X₁₁-X₁₂-X₁₃-X₁₃ₐ-X_{13b}-X_{13c}-X_{13d}-X₁₃ₑ-X_{13f}-X_{13g}-X₁₃ₕ-X₁₃ᵢ-X₁₃ⱼ-X₁₃ₖ-X₁₃ₗ-F₁₄-X₁₅-Z₁₆-X₁₇-X₁₈-X₁₉-X₂₀-X₂₁-X₂₁ₐ-X_{21b}-X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-X₂₁ₘ-X₂₁ₙ-X₂₁ₒ-X₂₁ₚ-X_{21q}-X₂₁ᵣ-X₂₁ₛ-X₂₁ₜ-D₂₂ (SEQ ID NO:360), w wherein each of X₂, X₄-X₁₃, X₁₅, and X₁₇-X₂₀ is, independently, any amino acid, wherein each of X₁₃ₐ-X₁₃ₗ is, independently, any amino acid or absent, wherein each of X₂₁ₐ-X₂₁ₜ is, independently, any amino acid or absent, and wherein Z₁₆ is selected from valine, aspartate, glycine, isoleucine, and leucine; wherein the variant aspartokinase includes an amino acid change at one or more of G₁, K₃, F₁₄, Z₁₆, or D₂₂ of SEQ ID NO:360. In various embodiments, the amino acid change is a change to an alanine.

In another aspect, the invention features an isolated nucleic acid encoding a variant bacterial aspartokinase polypeptide, wherein the aspartokinase polypeptide is a C. glutamicum aspartokinase polypeptide including an amino acid change in one or more of the following residues of SEQ ID NO:202: Glycine 208, Lysine 210, Phenylalanine 223, Valine 225, and Aspartate 236. In various embodiments, the amino acid change is a change to an alanine.

The invention also features a polypeptide encoded by the nucleic acid encoding a variant bacterial aspartokinase polypeptide.

The invention also features a bacterium including the nucleic acid encoding a variant bacterial aspartokinase polypeptide. In various embodiments, the bacterium is a coryneform bacterium. The bacterium can further comprise one or more nucleic acids encoding other variant bacterial polypeptides (e.g., variant bacterial polypeptides involved in amino acid production, e.g., variant bacterial polypeptides described herein). In various embodiments, the bacterium further comprises one or more nucleic acid molecules (e.g., recombinant nucleic acid molecules) encoding a polypeptide involved in amino acid production (e.g., a polypeptide that is heterologous or homologous to the host cell, or a variant thereof). In various embodiments, the bacterium further comprises mutations in an endogenous sequence that result in increased or decreased activity of a polypeptide involved in amino acid production (e.g., by mutation of an endogenous sequence encoding the polypeptide involved in amino acid production or a sequence that regulates expression of the polypeptide, e.g., a promoter sequence).

The invention also features a method for producing an amino acid, the method including: cultivating a genetically modified bacterium including the nucleic acid encoding a variant bacterial aspartokinase polypeptide under conditions in which the nucleic acid is expressed and that allow the amino acid to be produced, and collecting the culture. The culture can be fractionated (e.g., to remove cells and/or to obtain fractions enriched in the amino acid).

In another aspect, the invention features an isolated nucleic acid encoding a variant bacterial O-succinylhomoserine/acetylhomoserine (thiol)-lyase polypeptide (O-succinylhomoserine (thiol)-lyase). In various embodiments, the variant O-succinylhomoserine (thiol)-lyase exhibits reduced feedback inhibition relative to a wild-type form of the O-succinylhomoserine (thiol)-lyase polypeptide. In various embodiments, the nucleic acid encodes an O-succinylhomoserine (thiol)-lyase polypeptide with reduced feedback inhibition by S-adenosylmethionine. In various embodiments, the bacterial O-succinylhomoserine (thiol)-lyase polypeptide is chosen from: a Corynebacterium glutamicum O-succinylhomoserine (thiol)-lyase polypeptide, a Mycobacterium smegmatis O-succinylhomoserine (thiol)-lyase polypeptide, a Thermobifida fusca O-succinylhomoserine (thiol)-lyase polypeptide, an Amycolatopsis mediterranei O-succinylhomoserine (thiol)-lyase polypeptide, a Streptomyces coelicolor O-succinylhomoserine (thiol)-lyase polypeptide, an Erwinia chrysanthemi O-succinylhomoserine (thiol)-lyase polypeptide, a Shewanella oneidensis O-succinylhomoserine (thiol)-lyase polypeptide, a Mycobacterium tuberculosis O-succinylhomoserine (thiol)-lyase polypeptide, an Escherichia coli O-succinylhomoserine (thiol)-lyase polypeptide, a Corynebacterium acetoglutamicum O-succinylhomoserine (thiol)-lyase polypeptide, a Corynebacterium melassecola O-succinylhomoserine (thiol)-lyase polypeptide, a Corynebacterium thermoaminogenes O-succinylhomoserine (thiol)-lyase polypeptide, a Brevibacterium lactofermentum O-succinylhomoserine (thiol)-lyase polypeptide, a Brevibacterium lactis O-succinylhomoserine (thiol)-lyase polypeptide, and a Brevibacterium flavum O-succinylhomoserine (thiol)-lyase polypeptide.

In another aspect, the invention features an isolated nucleic acid encoding a variant bacterial O-succinylhomoserine (thiol)-lyase polypeptide, wherein the variant O-succinylhomoserine (thiol)-lyase polypeptide is a variant of an O-succinylhomoserine (thiol)-lyase polypeptide including the following amino acid sequence: G₁-X₂-K₃-X₄-X₅-X₆-X₇-X₈-X₉-X₁₀-X₁₁-X₁₂-X₁₃ₐ-X_{13b}-X_{13c}-X_{13d}-X₁₃ₑ-X_{13f}-X_{13g}-X₁₃ₕ-X₁₃ᵢ-X₁₃ⱼ-X₁₃ₖ-X₁₃ₗ-F₁₄-X₁₅-Z₁₆-X₁₇-X₁₈-X₁₉-X₂₀-X₂₁-X₂₁ₐ-X_{21b}-X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-X₂₁ₘ-X₂₁ₙ-X₂₁ₒ-X₂₁ₚ-X_{21q}-X₂₁ᵣ-X₂₁ₛ-X₂₁ₜ-D₂₂ (SEQ ID NO:360), wherein each of X₂, X₄-X₁₃, X₁₅, and X₁₇-X₂₀ is, independently, any amino acid, wherein each of X₁₃ₐ-X₁₃ₗ is, independently, any amino acid or absent, wherein each of X₂₁ₐ-X₂₁ₜ is, independently, any amino acid or absent, and wherein Z₁₆ is selected from valine, aspartate, glycine, isoleucine, and leucine; wherein the variant O-succinylhomoserine (thiol)-lyase polypeptide includes an amino acid change at one or more of G₁, K₃, F₁₄, Z₁₆, or D₂₂ of SEQ ID NO:360. In various embodiments, the amino acid change is a change to an alanine.

In another aspect, the invention features an isolated nucleic acid encoding a variant bacterial O-succinylhomoserine (thiol)-lyase polypeptide, wherein the variant O-succinylhomoserine (thiol)-lyase polypeptide is a C. glutamicum O-succinylhomoserine (thiol)-lyase polypeptide including an amino acid change in one or more of the following residues of SEQ ID NO:235: Glycine 72, Lysine 74, Phenylalanine 90, isoleucine 92, and Aspartate 105. In various embodiments, the amino acid change is a change to an alanine.

The invention also features a polypeptide encoded by a nucleic acid encoding a variant bacterial O-succinylhomoserine (thiol)-lyase polypeptide.

The invention also features a bacterium including a nucleic acid encoding a variant bacterial O-succinylhomoserine (thiol)-lyase polypeptide. In various embodiments, the bacterium is a coryneform bacterium. The bacterium can further comprise one or more nucleic acids encoding other variant bacterial polypeptides (e.g., variant bacterial polypeptides involved in amino acid production, e.g., variant bacterial polypeptides described herein).

The invention also features a method for producing L-methionine, the method including: cultivating a genetically modified bacterium including a nucleic acid encoding a variant bacterial O-succinylhomoserine (thiol)-lyase polypeptide under conditions in which the nucleic acid is expressed and that allow L-methionine to be produced, and collecting the culture. The culture can be fractionated (e.g., to remove cells and/or to obtain fractions enriched in L-methionine).

In another aspect, the invention features an isolated nucleic acid encoding a variant bacterial cystathionine beta-lyase polypeptide. In various embodiments, the variant cystathionine beta-lyase polypeptide exhibits reduced feedback inhibition relative to a wild-type form of the cystathionine beta-lyase polypeptide. In various embodiments, the nucleic acid encodes a cystathionine beta-lyase polypeptide with reduced feedback inhibition by S-adenosylmethionine. In various embodiments, the bacterial cystathionine beta-lyase polypeptide is chosen from: a Corynebacterium glutamicum cystathionine beta-lyase polypeptide, a Mycobacterium smegmatis cystathionine beta-lyase polypeptide, a Thermobifida fusca cystathionine beta-lyase polypeptide, an Amycolatopsis mediterranei cystathionine beta-lyase polypeptide, a Streptomyces coelicolor cystathionine beta-lyase polypeptide, an Erwinia chrysanthemi cystathionine beta-lyase polypeptide, a Shewanella oneidensis cystathionine beta-lyase polyp eptide, a Mycobacterium tuberculosis cystathionine beta-lyase polyp eptide, an Escherichia coli cystathionine beta-lyase polypeptide, a Corynebacterium acetoglutamicum cystathionine beta-lyase polypeptide, a Corynebacterium melassecola cystathione beta-lyase polypeptide, a Corynebacterium thermoaminogenes cystathionine beta-lyase polypeptide, a Brevibacterium lactofermentum cystathionine beta-lyase polypeptide, a Brevibacterium lactis cystathionine beta-lyase polypeptide, and a Brevibacteriumflavum cystathionine beta-lyase polypeptide.

In another aspect, the invention features an isolated nucleic acid encoding a variant bacterial cystathionine beta-lyase polypeptide, wherein the variant cystathionine beta-lyase polypeptide is a variant of a cystathionine beta-lyase polypeptide including the following amino acid sequence: G₁-X₂-X₃-X₄-X₅-X₆-X₇-X₈-X₉-X₁₀-X₁₁-X₁₂-X₁₃-X₁₃ₐ-X_{13b}-X_{13c}-X_{13d}-X₁₃ₑ-X_{13f}-X_{13g}-X₁₃ₕ-X₁₃ᵢ-X₁₃ⱼ-X₁₃ₖ-X₁₃ₗ-F₁₄-X₁₅-Z₁₆-X₁₇-X₁₈-X₁₉-X₂₀-X₂₁-X₂₁ₐ-X_{21b}-X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-X₂₁ₘ-X₂₁ₙ-X₂₁ₒ-X₂₁ₚ-X_{21q}-X₂₁ᵣ-X₂₁ₛ-X₂₁ₜ-D₂₂ (SEQ ID NO:360), wherein each of X₂, X₄-X₁₃, X₁₅, and X₁₇-X₂₀ is, independently, any amino acid, wherein each of X₁₃ₐ-X₁₃ₗ is, independently, any amino acid or absent, wherein each of X₂₁ₐ-X₂₁ₜ is, independently, any amino acid or absent, and wherein Z₁₆ is selected from valine, aspartate, glycine, isoleucine, and leucine; wherein the variant cystathionine beta-lyase includes an amino acid change at one or more of G₁, K₃, F₁₄, Z₁₆, or D₂₂ of SEQ ID NO:360. In various embodiments, the amino acid change is a change to an alanine.

In another aspect, the invention features an isolated nucleic acid encoding a variant bacterial cystathionine beta-lyase polypeptide, wherein the variant cystathionine beta-lyase polypeptide is a C. glutamicum cystathionine beta-lyase polypeptide including an amino acid change in one or more of the following residues of SEQ ID NO:217: Glycine 296, Lysine 298, Phenylalanine 312, Glycine 314 and Aspartate 335. In various embodiments, the amino acid change is a change to an alanine.

The invention also features a polypeptide encoded by a nucleic acid encoding a variant bacterial cystathionine beta-lyase.

The invention also features a bacterium including a nucleic acid encoding a variant bacterial cystathionine beta-lyase polypeptide. In various embodiments, the bacterium is a coryneform bacterium. The bacterium can further comprise one or more nucleic acids encoding other variant bacterial polypeptides (e.g., variant bacterial polypeptides involved in amino acid production, e.g., variant bacterial polypeptides described herein).

The invention also features a method for producing L-methionine, the method including:

cultivating a genetically modified bacterium including a nucleic acid encoding a variant bacterial cystathionine beta-lyase polypeptide under conditions in which the nucleic acid is expressed and that allow L-methionine to be produced, and collecting the culture. The culture can be fractionated (e.g., to remove cells and/or to obtain fractions enriched in L-methionine).

In another aspect, the invention features an isolated nucleic acid encoding a variant bacterial 5-methyltetrahydrofolate homocysteine methyltransferase polypeptide. In various embodiments, the variant 5-methyltetrahydrofolate homocysteine methyltransferase polypeptide exhibits reduced feedback inhibition relative to a wild-type form of the 5-methyltetrahydrofolate homocysteine methyltransferase polypeptide. In various embodiments, the nucleic acid encodes a 5-methyltetrahydrofolate homocysteine methyltransferase polypeptide with reduced feedback inhibition by S-adenosylmethionine polypeptide. In various embodiments, the bacterial 5-methyltetrahydrofolate homocysteine methyltransferase polypeptide is chosen from: a Corynebacterium glutamicum 5-methyltetrahydrofolate homocysteine methyltransferase polypeptide, a Mycobacterium smegmatis 5-methyltetrahydrofolate homocysteine methyltransferase polypeptide, a Thermobifida fusca 5-methyltetrahydrofolate homocysteine methyltransferase polypeptide, an Amycolatopsis mediterranei 5-methyltetrahydrofolate homocysteine methyltransferase polypeptide, a Streptomyces coelicolor 5-methyltetrahydrofolate homocysteine methyltransferase polypeptide, an Erwinia chrysanthemi 5-methyltetrahydrofolate homocysteine methyltransferase polypeptide, a Shewanella oneidensis 5-methyltetrahydrofolate homocysteine methyltransferase polypeptide, a Mycobacterium tuberculosis 5-methyltetrahydrofolate homocysteine methyltransferase polypeptide, an Escherichia coli 5-methyltetrahydrofolate homocysteine methyltransferase polypeptide, a Corynebacterium acetoglutamicum 5-methyltetrahydrofolate homocysteine methyltransferase polypeptide, a Corynebacterium melassecola 5-methyltetrahydrofolate homocysteine methyltransferase polypeptide, a Corynebacterium thermoaminogenes 5-methyltetrahydrofolate homocysteine methyltransferase polypeptide, a Brevibacterium lactofermentum 5-methyltetrahydrofolate homocysteine methyltransferase polypeptide, a Brevibacterium lactis 5-methyltetrahydrofolate homocysteine methyltransferase polypeptide, and a Brevibacterium flavum 5-methyltetrahydrofolate homocysteine methyltransferase polypeptide.

In another aspect, the invention features an isolated nucleic acid encoding a variant bacterial 5-methyltetrahydrofolate homocysteine methyltransferase polypeptide, wherein the variant 5-methyltetrahydrofolate homocysteine methyltransferase polypeptide is a variant of a 5-methyltetrahydrofolate homocysteine methyltransferase polypeptide including the following amino acid sequence: G₁-X₂ -K₃ -X₄ -X₅-X₆-X₇-X₈-X₉-X₁₀-X₁₁-X₁₂-X₁₃-X₁₃ₐ-X_{13b}-X_{13c}-X_{13d}-X₁₃ₑ-X_{13f}-X_{13g}-X₁₃ₕ-X₁₃ᵢ-X₁₃ⱼ-X₁₃ₖ-X₁₃ₗ-F₁₄-X₁₅-Z₁₆ SEQ ID NO: 362), wherein X is any amino acid, wherein each of X₁₃ₐ-X₁₃ₗ is, independently, any amino acid or absent, and wherein Z₁₆ is selected from valine, aspartate, glycine, isoleucine, and leucine; wherein the variant 5-methyltetrahydrofolate homocysteine methyltransferase polypeptide includes an amino acid change at one or more of G₁, K₃, F₁₄, or Z₁₆, of SEQ ID NO:362. In various embodiments, the amino acid change is a change to an alanine.

In another aspect, the invention features an isolated nucleic acid encoding a variant bacterial 5-methyltetrahydrofolate homocysteine methyltransferase polypeptide, wherein the variant 5-methyltetrahydrofolate homocysteine methyltransferase polypeptide is a C. glutamicum 5-methyltetrahydrofolate homocysteine methyltransferase polypeptide including an amino acid change in one or more of the following residues of SEQ ID NO:222:

Glycine 708, Lysine 710, Phenylalanine 725, and Leucine 727. In various embodiments, the amino acid change is a change to an alanine.

The invention also features a polypeptide encoded by the nucleic acid encoding a variant bacterial 5-methyltetrahydrofolate homocysteine methyltransferase.

The invention also features a bacterium including a nucleic acid encoding a variant bacterial 5-methyltetrahydrofolate homocysteine methyltransferase polypeptide. In various embodiments, the bacterium is a coryneform bacterium. The bacterium can further comprise one or more nucleic acids encoding other variant bacterial polypeptides (e.g., variant bacterial polypeptides involved in amino acid production, e.g., variant bacterial polypeptides described herein).

The invention also features a method for producing L-methionine, the method including: cultivating a genetically modified bacterium including a nucleic acid encoding a variant bacterial 5-methyltetrahydrofolate homocysteine methyltransferase polypeptide under conditions in which the nucleic acid is expressed and that allow L-methionine to be produced, and collecting the culture. The culture can be fractionated (e.g., to remove cells and/or to obtain fractions enriched in L-methionine).

In another aspect, the invention features an isolated nucleic acid encoding a variant bacterial S-adenosylmethionine synthetase polypeptide. In various embodiments, the variant S-adenosylmethionine synthetase polypeptide exhibits reduced feedback inhibition relative to a wild-type form of the S-adenosylmethionine synthetase polypeptide. In various embodiments, the nucleic acid encodes an S-adenosylmethionine synthetase polypeptide with reduced feedback inhibition by S-adenosylmethionine. In various embodiments, the bacterial S-adenosylmethionine synthetase polypeptide is chosen from: a Corynebacterium glutamicum S-adenosylmethionine synthetase polypeptide, a Mycobacterium smegmatis S-adenosylmethionine synthetase polypeptide, a Thermobifida fusca S-adenosylmethionine synthetase polypeptide, an Amycolatopsis mediterranei S-adenosylmethionine synthetase polypeptide, a Streptomyces coelicolor S-adenosylmethionine synthetase polypeptide, an Erwinia chrysanthemi S-adenosylmethionine synthetase polypeptide, a Shewanella oneidensis S-adenosylmethionine synthetase polypeptide, a Mycobacterium tuberculosis S-adenosylmethionine synthetase polypeptide, an Escherichia coli S-adenosylmethionine synthetase polypeptide, a Corynebacterium acetoglutamicum S-adenosylmethionine synthetase polypeptide, a Corynebacterium melassecola S-adenosylmethionine synthetase polypeptide, a Corynebacterium thermoaminogenes S-adenosylmethionine synthetase polypeptide, a Brevibacterium lactofermentum S-adenosylmethionine synthetase polypeptide, a Brevibacterium lactis S-adenosylmethionine synthetase polypeptide, and a Brevibacterium flavum S-adenosylmethionine synthetase polypeptide.

In another aspect, the invention features an isolated nucleic acid encoding a variant bacterial S-adenosylmethionine synthetase polypeptide, wherein the variant S-adenosylmethionine synthetase polypeptide is a variant of an S-adenosylmethionine synthetase polypeptide including the following amino acid sequence: G₁-X₂-K₃-X₄₋X₅-X₆-X₇₋X8-X₉-X₁₀-X₁₁-X₁₂-X₁₃-X₁₃ₐ-X_{13b}-X_{13c}-X_{13d}-X₁₃ₑ-X_{13f}-X_{13g}-X₁₃ₕ-X₁₃ᵢ-X₁₃ⱼ-X₁₃ₖ-X₁₃ₗ-F₁₄-X₁₅-Z₁₆-X₁₇-X₁₈-X₁₉-X₂₀-X₂₁-X₂₁ₐ-X_{21b}-X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-X₂₁ₘ-X₂₁ₙ-X₂₁ₒ-X₂₁ₚ-X_{21q}-X₂₁ᵣ-X₂₁ₛ-X₂₁ₜ-D₂₂ (SEQ ID NO:360), wherein each of X₂, X₄-X₁₃, X₁₅, and X₁₇-X₂₀ is, independently, any amino acid,wherein each of X₁₃ₐ-X₁₃ₗ is, independently, any amino acid or absent, wherein each of X₂₁ₐ-X₂₁ₜ is, independently, any amino acid or absent, and wherein Z₁₆ is selected from valine, aspartate, glycine, isoleucine, and leucine; wherein the variant S-adenosylmethionine synthetase polypeptide includes an amino acid change at one or more of G₁, K₃, F₁₄, Z₁₆, or D₂₂ of SEQ ID NO:360. In various embodiments, the amino acid change is a change to an alanine.

In another aspect, the invention features an isolated nucleic acid encoding a variant bacterial S-adenosylmethionine synthetase polypeptide, wherein the variant S-adenosylmethionine synthetase polypeptide is a C. glutamicum S-adenosylmethionine synthetase polypeptide including an amino acid change in one or more of the following residues of SEQ ID NO:215: Glycine 263, Lysine 265, Phenylalanine 282, Glycine 284, and Aspartate 291.

In various embodiments, the amino acid change is a change to an alanine.

The invention also features a polypeptide encoded by a nucleic acid encoding a variant bacterial S-adenosylmethionine synthetase polypeptide.

The invention also features a bacterium including a nucleic acid encoding a variant bacterial S-adenosylmethionine synthetase polypeptide. In various embodiments, the bacterium is a coryneform bacterium. The bacterium can further comprise one or more nucleic acids encoding other variant bacterial polypeptides (e.g., variant bacterial polypeptides involved in amino acid production, e.g., variant bacterial polypeptides described herein).

The invention also features a method for producing L-methionine, the method including: cultivating a genetically modified bacterium including a nucleic acid encoding a variant bacterial S-adenosylmethionine synthetase polypeptide under conditions in which the nucleic acid is expressed and that allow L-methionine to be produced, and collecting the culture. The culture can be fractionated (e.g., to remove cells and/or to obtain fractions enriched in L-methionine).

In another aspect, the invention features an isolated nucleic acid encoding a variant bacterial homoserine kinase polypeptide. In various embodiments, the variant homoserine kinase polypeptide exhibits reduced feedback inhibition relative to a wild-type form of the bacterial homoserine kinase polypeptide. In various embodiments, the nucleic acid encodes a homoserine kinase polypeptide with reduced feedback inhibition by S-adenosylmethionine. In various embodiments, the bacterial homoserine kinase polypeptide is chosen from: a Corynebacterium glutamicum homoserine kinase polypeptide, a Mycobacterium smegmatis homoserine kinase polypeptide, a Thermobifida fusca homoserine kinase polypeptide, an Amycolatopsis mediterranei homoserine kinase polypeptide, a Streptomyces coelicolor homoserine kinase polypeptide, an Erwinia chrysanthemi homoserine kinase polypeptide, a Shewanella oneidensis homoserine kinase polypeptide, a Mycobacterium tuberculosis homoserine kinase polypeptide, an Escherichia coli homoserine kinase polypeptide, a Corynebacterium acetoglutamicum homoserine kinase polypeptide, a Corynebacterium melassecola homoserine kinase polypeptide, a Corynebacterium thermoaminogenes homoserine kinase polypeptide, a Brevibacterium lactofermentum homoserine kinase polypeptide, a Brevibacterium lactis homoserine kinase polypeptide, and a Brevibacterium flavum homoserine kinase polypeptide.

In another aspect, the invention features an isolated nucleic acid encoding a variant bacterial homoserine kinase polypeptide, wherein the homoserine kinase polypeptide is a C. glutamicum homoserine kinase polypeptide including an amino acid change in one or more of the following residues of SEQ ID NO:364: Glycine 160, Lysine 161, Phenylalanine 186, Alanine 188, and Aspartate 205. In various embodiments, the amino acid change is a change to an alanine, wherein the original residue is other than an alanine.

The invention also features a polypeptide encoded by the nucleic acid encoding a variant bacterial homoserine kinase.

The invention also features a bacterium including the nucleic acid encoding a variant bacterial homoserine kinase polypeptide. In various embodiments, the bacterium is a coryneform bacterium. The bacterium can further include one or more nucleic acids encoding other variant bacterial polypeptides (e.g., variant bacterial polypeptides involved in amino acid production, e.g., variant bacterial polypeptides described herein).

The invention also features a method for producing an amino acid, the method including: cultivating a genetically modified bacterium including the nucleic acid encoding a variant bacterial homoserine kinase polypeptide under conditions in which the nucleic acid is expressed and that allow the amino acid to be produced, and collecting the culture. The culture can be fractionated (e.g., to remove cells and/or to obtain fractions enriched in the amino acid).

In another aspect, the invention features a bacterium including two or more of the following: a nucleic acid encoding a variant bacterial homoserine O-acetyltransferase polypeptide; a nucleic acid encoding a variant bacterial O-acetylhomoserine sulfhydrylase; a nucleic acid encoding a variant bacterial McbR gene product polypeptide; a nucleic acid encoding a variant bacterial aspartokinase polypeptide; a nucleic acid encoding a variant bacterial O-succinylhomoserine (thiol)-lyase polypeptide; a nucleic acid encoding a variant bacterial cystathione beta-lyase polypeptide; a nucleic acid encoding a variant bacterial 5-methyltetrahydrofolate homocysteine methyltransferase polypeptide; and a nucleic acid encoding a variant bacterial S-adenosylmethionine synthetase polypeptide.

In various embodiments, the bacterium comprises a nucleic acid encoding a variant bacterial homoserine O-acetyltransferase and a nucleic acid encoding a variant bacterial O-acetylhomoserine sulfhydrylase. In certain embodiments, at least one of the variant bacterial polypeptides have reduced feedback inhibition (e.g., relative to a wild-type form of the polypeptide).

In another aspect, the invention features a bacterium including two or more of the following: (a) a nucleic acid encoding a variant bacterial homoserine O-acetyltransferase polypeptide, wherein the variant homoserine O-acetyltransferase polypeptide is a variant of a homoserine O-acetyltransferase polypeptide including the following amino acid sequence: G₁-X-X₂-X₃-X₄-X₅-X₆-X₇-X₈-X₉-X₁₀-X₁₁-X₁₂-X₁₃-X₁₃ₐ-X_{13b}-X_{13c}-X_{13d}-X₁₃ₑ-X_{13f}-X_{13g}-X₁₃ₕ-X₁₃ᵢ-X₁₃ⱼ-X₁₃ₖ-X₁₃ₗ-F₁₄-X₁₅-Z₁₆-X₁₇-X₁₈-X₁₉-X₂₀-X₂₁-X₂₁ₐ-X_{21b}-X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-X₂₁ₘ-X₂₁ₙ-X₂₁ₒ-X₂₁ₚ-X_{21q}-X₂₁ᵣ-X₂₁ₛ-X₂₁ₜ-D₂₂ (SEQ ID NO:360), wherein each of X₂, X₄-X₁₃, X₁₅, and X₁₇-X₂₀ is, independently, any amino acid, wherein each of X₁₃ₐ-X₁₃ₗ is, independently, any amino acid or absent, wherein each of X₂₁ₐ-X₂₁ₜ is, independently, any amino acid or absent, and wherein Z₁₆ is selected from valine, aspartate, glycine, isoleucine, and leucine; wherein the variant homoserine O-acetyltransferase polypeptide includes an amino acid change at one or more of G₁, K₃, F₁₄, Z₁₆, or D₂₂ of SEQ ID NO:360; (b) a nucleic acid encoding a variant bacterial O-acetylhomoserine sulfhydrylase polypeptide, wherein the variant O-acetylhomoserine sulfhydrylase polypeptide is a variant of an O-acetylhomoserine sulfhydrylase polypeptide including the following amino acid sequence: G₁-X₂-K₃-X₄-X₅-X₆-X₇-X₈-X₉-X₁₀-X₁₁-X₁₂-X₁₃-X₁₃ₐ-X_{13b}-X_{13c}-X_{13d}-X₁₃ₑ-X_{13f}-X_{13g}-X₁₃ₕ-X₁₃ᵢ-X₁₃ⱼ-X₁₃ₖ-X₁₃ₗ-F₁₄-X₁₅-Z₁₆-X₁₇-X₁₈-X₁₉-X₂₀-X₂₁-X₂₁ₐ-X_{21b}-X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-X₂₁ₘ-X₂₁ₙ-X₂₁ₒ-X₂₁ₚ-X_{21q}-X₂₁ᵣ-X₂₁ₛ-X₂₁ₜ-D₂₂ (SEQ ID NO:360), wherein each of X₂, X₄-X₁₃, X₁₅, and X₁₇-X₂₀ is, independently, any amino acid, wherein each of X₁₃ₐ-X₁₃ₗ is, independently, any amino acid or absent, wherein each of X₂₁ₐ-X₂₁ₜ is, independently, any amino acid or absent, and wherein Z₁₆ is selected from valine, aspartate, glycine, isoleucine, and leucine; wherein the variant O-acetylhomoserine sulfhydrylase polypeptide includes an amino acid change at one or more of G₁, K₃, F₁₄, Z₁₆, or D₂₂ of SEQ ID NO:360; and (c) a nucleic acid encoding a variant bacterial O-acetylhomoserine sulfhydrylase polypeptide, wherein the variant O-acetylhomoserine sulfhydrylase polypeptide is a variant of a O-acetylhomoserine sulfhydrylase polypeptide including the following amino acid sequence: L₁-X₂-X₃-G₄-G₅-X₆-F₇-X₈-X₉-X₁₀-X₁₁ (SEQ ID NO:361), wherein X is any amino acid, wherein X₈ is selected from valine, leucine, isoleucine, and aspartate, and wherein X₁₁₁ is selected from valine, leucine, isoleucine, phenylalanine, and methionine; wherein the variant of the bacterial protein includes an amino acid change at one or more of L₁, G₄, X₈, X₁₁ of SEQ ID NO:361.

In another aspect, the invention features a bacterium including two or more of the following: (a) a nucleic acid encoding a variant bacterial homoserine O-acetyltransferase polypeptide, wherein the variant homoserine O-acetyltransferase polypeptide is a C. glutamicum homoserine O-acetyltransferase polypeptide including an amino acid change in one or more of the following residues of SEQ ID NO:212: Glycine 231, Lysine 233, Phenylalanine 251, and Valine 253; (b) a nucleic acid encoding a variant bacterial homoserine O-acetyltransferase polypeptide, wherein the variant homoserine O-acetyltransferase polypeptide is a T. fusca homoserine O-acetyltransferase polypeptide including an amino acid change in one or more of the following residues of SEQ ID NO:24: Glycine 81, Aspartate 287, Phenylalanine 269; (c) a nucleic acid encoding a variant bacterial homoserine O-acetyltransferase polypeptide, wherein the variant homoserine O-acetyltransferase polypeptide is an E. coli homoserine O-acetyltransferase polypeptide including an amino acid change at Glutamate 252 of SEQ ID NO:213; (d) a nucleic acid encoding a variant bacterial homoserine O-acetyltransferase polypeptide, wherein the variant homoserine O-acetyltransferase polypeptide is a mycobacterial homoserine O-acetyltransferase polypeptide including an amino acid change in a residue corresponding to one or more of the following residues of M. leprae homoserine O-acetyltransferase polypeptide set forth in SEQ ID NO:23: Glycine 73, Aspartate 278, and Tyrosine 260; (e) a nucleic acid encoding a variant bacterial homoserine O-acetyltransferase polypeptide, wherein the variant homoserine O-acetyltransferase polypeptide is an M. tuberculosis homoserine O-acetyltransferase polypeptide including an amino acid change in one or more of the following residues of SEQ ID NO:22: Glycine 73, Tyrosine 260, and Aspartate 278; (f) a nucleic acid encoding a variant bacterial O-acetylhomoserine sulfhydrylase polypeptide, wherein the variant O-acetylhomoserine sulfhydrylase polypeptide is a C. glutamicum O-acetylhomoserine sulfhydrylase polypeptide including an amino acid change in one or more of the following residues of SEQ ID NO:214: Glycine 227, Leucine 229, Aspartate 231, Glycine 232, Glycine 233, Phenylalanine 235, Aspartate 236, Valine 239, Phenylalanine 368, Aspartate 370, Aspartate 383, Glycine 346, and Lycine 348; and (g) a nucleic acid encoding a variant bacterial O-acetylhomoserine sulfhydrylase polypeptide, wherein the variant O-acetylhomoserine sulfhydrylase polypeptide is a T. fusca O-acetylhomoserine sulfhydrylase polypeptide including an amino acid change in one or more of the following residues of SEQ ID NO:25: Glycine 240, Aspartate 244, Phenylalanine 379, and Aspartate 394.

In another aspect, the invention features a bacterium including a nucleic acid encoding an episomal homoserine O-acetyltransferase polypeptide and an episomal O-acetylhomoserine sulfhydrylase polypeptide. In various embodiments, the bacterium is a Corynebacterium. In various embodiments, the episomal homoserine O-acetyltransferase polypeptide and the episomal O-acetylhomoserine sulfhydrylase polypeptide are of the same species as the bacterium (e.g., both are of C. glutamicum). In various embodiments, the episomal homoserine O-acetyltransferase polypeptide and the episomal O-acetylhomoserine sulfhydrylase polypeptide are of a different species than the bacterium. In various embodiments, the episomal homoserine O-acetyltransferase polypeptide is a variant of a bacterial homoserine O-acetyltransferase polypeptide with reduced feedback inhibition relative to a wild-type form of the homoserine O-acetyltransferase polypeptide. In various embodiments, the O-acetylhomoserine sulfhydrylase polypeptide is a variant of a bacterial O-acetylhomoserine sulfhydrylase polypeptide with reduced feedback inhibition relative to a wild-type form of the O-acetylhomoserine sulfhydrylase polypeptide.

“Aspartic acid family of amino acids and related metabolites” encompasses L-aspartate, β-aspartyl phosphate, L-aspartate-β-semialdehyde, L-2,3-dihydrodipicolinate, L-Δ¹-piperideine-2,6-dicarboxylate, N-succinyl-2-amino-6-keto-L-pimelate, N-succinyl-2, 6-L, L-diaminopimelate, L, L-diaminopimelate, D, L-diaminopimelate, L-lysine, homoserine, O-acetyl-L-homoserine, O-succinyl-L-homoserine, cystathionine, L-homocysteine, L-methionine, S-adenosyl-L-methionine, O-phospho-L-homoserine, threonine, 2-oxobutanoate, (S)-2-aceto-2-hydroxybutanoate, (S)-2-hydroxy-3-methyl-3-oxopentanoate, (R)-2,3-Dihydroxy-3-methylpentanoate, (R)-2-oxo-3-methylpentanoate, L-isoleucine, L-asparagine. In various embodiments the aspartic acid family of amino acids and related metabolites encompasses aspartic acid, asparagine, lysine, threonine, methionine, isoleucine, and S-adenosyl-L-methionine. A polypeptide or functional variant thereof with “reduced feedback inhibition” includes a polypeptide that is less inhibited by the presence of an inhibitory factor as compared to a wild-type form of the polypeptide or a polypeptide that is less inhibited by the presence of an inhibitory factor as compared to the corresponding endogenous polypeptide expressed in the organism into which the variant has been introduced. For example, a wild-type aspartokinase from E. coli or C. glutamicum may have 10-fold less activity in the presence of a given concentration of lysine, or lysine plus threonine, respectively. A variant with reduced feedback inhibition may have, for example, 5-fold less, 2-fold less, or wild-type levels of activity in the presence of the same concentration of lysine.

A “functional variant” protein is a protein that is capable of catalyzing the biosynthetic reaction catalyzed by the wild-type protein in the case where the protein is an enzyme, or providing the same biological function of the wild-type protein when that protein is not catalytic. For instance, a functional variant of a protein that normally regulates the transcription of one or more genes would still regulate the transcription of one or more of the same genes when transformed into a bacterium. In certain embodiments, a functional variant protein is at least partially or entirely resistant to feedback inhibition by an amino acid. In certain embodiments, the variant has fewer than 20, 15, 10, 9, 8, 7, 6, 5, 4, 3, or 1 amino acid changes compared to the wild-type protein. In certain embodiments, the amino acid changes are conservative changes. A variant sequence is a nucleotide or amino acid sequence corresponding to a variant polypeptide, e.g., a functional variant polypeptide.

An amino acid that is “corresponding” to an amino acid in a reference sequence occupies a site that is homologous to the site in the reference sequence. Corresponding amino acids can be identified by alignment of related sequences.

As used herein, a “heterologous” nucleic acid or protein is meant to encompass a nucleic acid or protein, or functional variant of a nucleic acid or protein, of an organism (species) other than the host organism (species) used for the production of members of the aspartic acid family of amino acids and related metabolites. In certain embodiments, when the host organism is a coryneform bacteria the heterologous gene will not be obtained from E. coli. In other specific embodiments, when the host organism is E. coli the heterologous gene will not be obtained from a coryneform bacteria.

“Gene”, as used herein, includes coding, promoter, operator, enhancer, terminator, co-transcribed (e.g., sequences from an operon), and other regulatory sequences associated with a particular coding sequence.

As used herein, a “homologous” nucleic acid or protein is meant to encompass a nucleic acid or protein, or functional variant of a nucleic acid or protein, of an organism that is the same species as the host organism used for the production of members of the aspartic acid family of amino acids and related metabolites.

As known to those skilled in the art, certain substitutions of one amino acid for another may be tolerated at one or more amino acid residues of a wild-type enzyme without eliminating the activity or function of the enzyme. As used herein, the term “conservative substitution” refers to the exchange of one amino acid for another in the same conservative substitution grouping in a protein sequence. Conservative amino acid substitutions are known in the art and are generally based on the relative similarity of the amino acid side-chain substituents, for example, their hydrophobicity, hydrophilicity, charge, size, and the like. In one embodiment, conservative substitutions typically include substitutions within the following groups: Group 1: glycine, alanine, and proline; Group 2: valine, isoleucine, leucine, and methionine; Group 3: aspartic acid, glutamic acid, asparagine, glutamine; Group 4: serine, threonine, and cysteine; Group 5: lysine, arginine, and histidine; Group 6: phenylalanine, tyrosine, and tryptophan. Each group provides a listing of amino acids that may be substituted in a protein sequence for any one of the other amino acids in that particular group.

There are several criteria used to establish groupings of amino acids for conservative substitution. For example, the importance of the hydropathic amino acid index in conferring interactive biological function on a protein is generally understood in the art (Kyte and Doolittle, Mol. Biol. 157:105-132 (1982). It is known that certain amino acids may be substituted for other amino acids having a similar hydropathic index or score and still retain a similar biological activity. Amino acid hydrophilicity is also used as a criterion for the establishment of conservative amino acid groupings (see, e.g., U.S. Patent No. 4,554,101).

Information relating to the substitution of one amino acid for another is generally known in the art (see, e.g., Introduction to Protein Architecture: The Structural Biology of Proteins, Lesk, A. M., Oxford University Press; ISBN: 0198504748; Introduction to Protein Structure, Branden, C.-I., Tooze, J., Karolinska Institute, Stockholm, Sweden (Jan. 15, 1999); and Protein Structure Prediction: Methods and Protocols (Methods in Molecular Biology), Webster, D. M.(Editor), August 2000, Humana Press, ISBN: 0896036375).

In some embodiments, the nucleic acid and/or protein sequences of a heterologous sequence and/or host strain gene will be compared, and the homology can be determined. Homology comparisons can be used, for example, to identify corresponding amino acids. The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm. For example, the percent identity between two nucleotide sequences can be determined using the algorithm of Needleman and Wunsch ((1970) J. Mol. Biol. 48:444-453) algorithm which has been incorporated into the GAP program in the GCG software package, using either a Blosum 62 matrix and a gap weight of 12, a gap extend penalty of 4, and a frameshift gap penalty of 5.

Generally, to determine the percent identity of two nucleic acid or protein sequences, the sequences are aligned for optimal comparison purposes (e.g., gaps can be introduced in one or both of a first and a second nucleic acid or amino acid sequence for optimal alignment and non-homologous sequences can be disregarded for comparison purposes). The length of a test sequence aligned for comparison purposes can be at least 30%, 40%, 50%, 60%, 70%, 80%, 90%, 100% of the length of the reference sequence. The nucleotides or amino acids at corresponding nucleotide or amino acid positions are then compared. When a position in the first sequence is occupied by the same nucleotide or amino acid as the corresponding position in the second sequence, then the molecules are identical at that position (as used herein “identity” is equivalent to “homology”).

The protein sequences described herein can be used as a “query sequence” to perform a search against a database of non-redundant sequences, for example. Such searches can be performed using the BLASTP and TBLASTN programs (version 2.0) of Altschul, et al. (1990) J. Mol. Biol. 215:403-10. BLAST protein searches can be performed with the BLASTP program, using, for example, the Blosum 62 matrix, a wordlength of 3, and a gap existence cost of 11 and a gap extension penalty of 1. Software for performing BLAST analyses is publicly available through the National Center for Biotechnology Information, and default paramenter can be used. Sequences described herein can also be used as query sequences in TBLASTN searches, using specific or default parameters.

The nucleic acid sequences described herein can be used as a “query sequence” to perform a search against a database of non-redundant sequences, for example. Such searches can be performed using the BLASTN and BLASTX programs (version 2.0) of Altschul, et al. (1990) J. Mol. Biol. 215:403-10. BLAST nucleotide searches can be performed with the BLASTN program, score=100, wordlength=11 to evaluate identity at the nucleic acid level. BLAST protein searches can be performed with the BLASTX program, score=50, wordlength=3 to evaluate identity at the protein level. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., BLASTX and BLASTN) can be used. Alignment of nucleotide sequences for comparison can also be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Nucleic acid sequences can be analyzed for hybridization properties. As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. Specific hybridization conditions referred to herein are as follows: 1) low stringency hybridization conditions in 6X sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one, two, three, four or more washes in 0.2×SSC, 0.1% SDS at 65° C.) very high stringency hybridization conditions are 0.5M sodium phosphate, 7% SDS at 65° C., followed by one or more washes at 0.2×SSC, 1% SDS at 65° C. Very high stringency conditions (at least 4 or more washes) are the preferred conditions and the ones that should be used unless otherwise specified.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1. is a diagram of the biosynthesis of aspartate amino acid family.

FIG. 2. is a diagram of the methionine biosynthetic pathway.

FIG. 3. is a restriction map of plasmid MB3961 (vector backbone plasmid).

FIG. 4. is a restriction map of plasmid MB4094 (vector backbone plasmid).

FIG. 5. is a restriction map of plasmid MB4083 (hom-thrB deletion construct).

FIG. 6. is a restriction map of plasmid MB4084 (thrB deletion construct).

FIG. 7. is a restriction map of plasmid MB4165 (mcbR deletion construct).

FIG. 8. is a restriction map of plasmid MB4169 (hom-thrB deletion/gpd-M. smegmatis lysC(T311I)-asd replacement construct).

FIG. 9. is a restriction map of plasmid MB4192 (hom-thrB deletion/gpd-S. coelicolor hom(G362E) replacement construct.

FIG. 10. is a restriction map of plasmid MB4276 (pck deletion/gpd-M. smegmatis lysC(T311I)-asd replacement construct).

FIG. 11. is a restriction map of plasmid MB4286 (mcbR deletion/trcRBS-T. fusca metA replacement construct).

FIG. 12A. is a restriction map of plasmid MB4287 (mcbR deletion/trcRBS-C. glutamicum metA (K233A)-metB replacement construct).

FIG. 12B. is a depiction of the nucleotide sequence of the DNA sequence in MB4278 (trcRBS-C. glutamicum metA YH) that spans from the trcRBS promoter to the stop of the metH gene.

FIG. 13 is a graph depicting the results of an assay to determine in vitro O-acetyltransferase activity of C. glutamicum MetA from two C. glutamicum strains, MA-442 and MA-449, in the presence and absence of IPTG.

FIG. 14 is a graph depicting the results of an assay to determine sensitivity of MetA in C. glutamicum strain MA-442 to inhibition by methionine and S-AM.

FIG. 15 is a graph depicting the results of an assay to determine the in vitro O-acetyltransferase activity of T. fusca MetA expressed in C. glutamicum strains MA-456, MA570, MA-578, and MA-479. Rate is a measure of the change in OD412 divided by time per nanograms of protein.

FIG. 16 is a graph depicting the results of an assay to determine in vitro MetY activity of T. fusca MetY expressed in C. glutamicum strains MA-456 and MA-570. Rate is defined as the change in OD412 divided by time per nanograms of protein.

FIG. 17. is a graph depicting the results of an assay to determine lysine production in C. glutamicum and B. lactofermentum strains expressing heterologous wild-type and mutant lysC variants.

FIG. 18 is a graph depicting results from an assay to determine lysine and homoserine production in C. glutamicum strain, MA-0331 in the presence and absence of the S. coelicolor hom G362E variant.

FIG. 19. is a graph depicting results from any assay to determine asparate concentrations in C. glutamicum strains MA-0331 and MA-0463 in the presence and absence of E chrysanthemi ppc.

FIG. 20 is a graph depicting results from an assay to determine lysine production in C. glutamicum strains MA-0331 and MA-0463 transformed with heterologous wild-type dapA genes.

FIG. 21 is a graph depicting results from an assay to determine metabolite levels in C. glutamicum strain MA-1378 and its parent strains.

FIG. 22 is a graph depicting results from an assay to determine homoserine and O-acetylhomoserine levels in C. glutamicum strains MA-0428, MA-0579, MA-1351, MA-1559 grown in the presence or absence of IPTG. IPTG induces expression of the episomal plasmid borne T. fusca metA gene.

FIG. 23. is a graph depicting results from an assay to determine metabolite levels in C. glutamicum strain MA-1559 and its parent strains.

FIG. 24 is a graph depicting methionine concentrations in broths from fermentations of two C. glutamicum strains, MA-622, and MA-699, which express a MetA K233A mutant polypeptide. Production by cells cultured in the presence and absence of IPTG is depicted.

FIG. 25 is a graph depicting methionine concentrations in broths from fermentations of two C. glutamicum strains, MA-622 and MA-699, expressing a MetY D23 1A mutant polypeptide. Production by cells cultured in the presence and absence of IPTG is depicted.

FIG. 26 is a graph depicting methionine concentrations in broths from fermentations of two C. glutamicum strains, MA-622 and MA-699, expressing a C. glutamicum MetY G232A mutant polypeptide. Production by cells cultured in the presence and absence of IPTG is depicted.

FIG. 27 is a graph depicting results from an assay to determine metabolite levels in C. glutamicum strains MA-1 906, MA-2028, MA-1 907, and MA-2025. Strains were grown in the presence and absence of IPTG.

FIG. 28 is a graph depicting results from an assay to determine metabolite levels in C. glutamicum strains MA-1667 and MA-1743. Strains were grown in the presence and absence of IPTG.

FIG. 29 is a graph depicting results from an assay to determine metabolite levels in C. glutamicum strains MA-0569, MA-1688, MA-1421, and MA-1790. Strains were grown in the absence and/or presence of IPTG.

FIG. 30 is a graph depicting results from an assay to determine metabolite levels in C. glutamicum strain MA-1 668 and its parent strains.

### DETAILED DESCRIPTION

The invention provides nucleic acids and modified bacteria that comprise nucleic acids encoding proteins that improve fermentative production of aspartate-derived amino acids and intermediate compounds. In particular, nucleic acids and bacteria relevant to the production of L-aspartate, L-lysine, L-methionine, S-adenosyl-L-methionine, threonine, L-isoleucine, homoserine, O-acetyl homoserine, homocysteine, and cystathionine are disclosed. The nucleic acids include genes that encode metabolic pathway proteins that modulate the biosynthesis of these amino acids, intermediates, and related metabolites either directly (e.g., via enzymatic conversion of intermediates) or indirectly (e.g., via transcriptional regulation of enzyme expression or regulation of amino acid export). The nucleic acid sequences encoding the proteins can be derived from bacterial species other than the host organism (species) used for the production of members of the aspartic acid family of amino acids and related metabolites. The invention also provides methods for producing the bacteria and the amino acids, including the production of amino acids for use in animal feed additives.

Modification of the sequences of certain bacterial proteins involved in amino acid production can lead to increased yields of amino acids. Regulated (e.g., reduced or increased) expression of modified or unmodified (e.g., wild type) bacterial enzymes can likewise enhance amino acid production. The methods and compositions described herein apply to bacterial proteins that regulate the production of amino acids and related metabolites, (e.g., proteins involved in the metabolism of methionine, threonine, isoleucine, aspartate, lysine, cysteine and sulfur), and nucleic acids encoding these proteins. These proteins include enzymes that catalyze the conversion of intermediates of amino acid biosynthetic pathways to other intermediates and/or end product, and proteins that directly regulate the expression and/or function of such enzymes. Target proteins for manipulation include those enzymes that are subject to various types of regulation such as repression, attenuation, or feedback-inhibition. Amino acid biosynthetic pathways in bacterial species, information regarding the proteins involved in these pathway, links to sequences of these proteins, and other related resources for identifying proteins for manipulation and/or expression as described herein can be accessed through linked databases described by Error! Hyperlink reference not valid.Bono et al., Genome Research, 8:203-210, 30 1998.

Strategies to manipulate the efficiency of amino acid biosynthesis for commercial production include overexpression, underexpression (including gene disruption or replacement), and conditional expression of specific genes, as well as genetic modification to optimize the activity of proteins. It is possible to reduce the sensitivity of biosynthetic enzymes to inhibitory stimuli, e.g., feedback inhibition due to the presence of biosynthetic pathway end products and intermediates. For example, strains used for commercial production of lysine derived from either coryneform bacteria or Escherichia coli typically display relative insensitivity to feedback inhibition by lysine. Useful coryneform bacterial strains are also relatively resistant to inhibition by threonine. Novel methods and compositions described herein result in enhanced amino acid production. While not bound by theory, these methods and compositions may result in enzymes that are enhanced due to reduced feedback inhibition in the presence of S-adenosylmethionine (S-AM) and/or methionine. Exemplary target genes for manipulation are bacterial dapA, hom, thrB, ppc, pyc, pck, metE, glyA, metA, metY, mcbR, lysC, asd, metB, metC, metH, and metK genes. These target genes can be manipulated individually or in various combinations.

In certain embodiments, it is useful to engineer strains such that the activity of particular genes is reduced (e.g., by mutation or deletion of an endogenous gene). For example, stains with reduced activity of one or more of hom, thrB, pck, or mcbR gene products can exhibit enhanced production of amino acids and related intermediates.

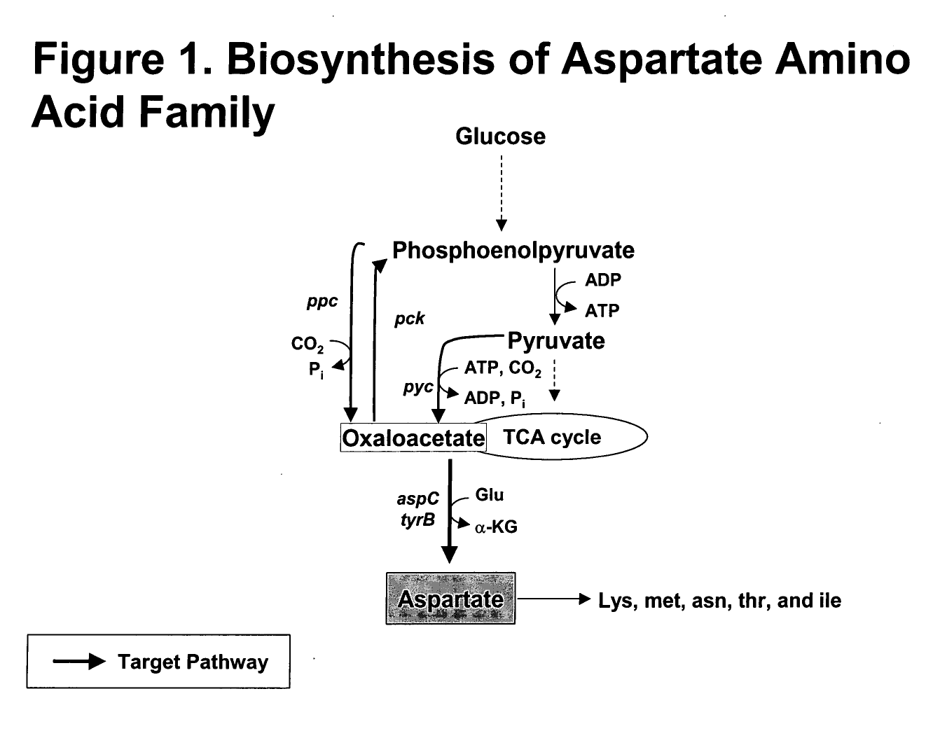

Two central carbon metabolism enzymes that direct carbon flow towards the aspartic acid family of amino acids and related metabolites include phosphoenolpyruvate carboxylase (Ppc) and pyruvate carboxylase (Pyc). The initial steps of biosynthesis of aspartatic acid family amino acids are diagrammed in FIG. 1. Both enzymes catalyze the formation of oxaloacetate, a tricarboxylic acid (TCA) cycle component that is transaminated to aspartic acid. Aspartokinase (which is encoded by lysC in coryneform bacteria) catalyzes the first enzyme reaction in the aspartic acid family of amino acids, and is known to be regulated by both feedback-inhibition and repression. Thus, deregulation of this enzyme is critical for the production of any of the commercially important amino acids and related metabolites of the aspartic acid amino acid pathway (e.g. aspartic acid, asparagine, lysine, methionine, S-adenosyl-L-methionine, threonine, and isoleucine). As critical enzymes for regulating carbon flow towards amino acids derived from aspartate, overexpression (by increasing copy number and/or the use of strong promoters) and/or deregulation of each or both of these enzymes can enhance production of the amino acids listed above.

Other biosynthetic enzymes can be employed to enhance production of specific amino acids. Examples of enzymes involved in L-lysine biosynthesis include: dihydrodipicolinate synthase (DapA), dihydrodipicolinate reductase (DapB), diaminopimelate dehydrogenase (Ddh), and diaminopimelate decarboxylase (LysA). A list of enzymes involved in lysine biosynthesis is provided in Table 1. Overexpression and/or deregulation of each of these enzymes can enhance production of lysine. Overexpression of biosynthetic enzymes can be achieved by increasing copy number of the gene of interest and/or operably linking the gene to apromoter optimal for expression, e.g., a strong or conditional promoter.

Lysine productivity can be enhanced in strains overexpressing general and specific regulatory enzymes. Specific amino acid substitutions in aspartokinase and dihydrodipicolinate synthase in E. coli can lead to increased lysine production by reducing feedback inhibition. Enhanced expression of lysC and/or dapA (either wild-type or feedback-insensitive alleles) can. ncrease lysine production. Similarly, deregulated alleles of heterologous lysC and dapA genes can be expressed in a strain of coryneform bacteria such as Corynebacterium glutamicum. Likewise, overexpression of eitherpyc or ppc can enhance lysine production.

TABLE 1
Genes and enzymes involved in lysine biosynthesis

| Gene | Enzyme | Comment |
|---|---|---|
| Pyc | Pyruvate Carboxylase | Anaplerotic reaction |
| Ppc | Phosphoenolpyruvate | Anaplerotic reaction |
| | Carboxylase | |
| AspC | Aspartate | Converts OAA to Aspartic acid. |
| | Aminotransferase | |
| LysC | Aspartate Kinase | Depending upon source species, |
| | (III) | feedback-inhibited by lysine |
| | | or lysine plus threonine, and |
| | | in some strains, repressed by |
| | | lysine. |
| Asd | Aspartic Semialdehyde | |
| | Dehydrogenase | |
| Hom | Homoserine | Key branch-point between lysine |
| | Dehydrogenase | and methionine/threonine. |
| DapA | Dihydrodipicolinate | Catalyzes first committed step |
| | Synthase | in lysine biosynthesis. Is |
| | | inhibited by lysine in E. coli. |
| DapB | Dihydrodipicolinate | |
| | Reductase | |
| DapC | N-succinyl-LL- | |
| | diaminopimelate | |
| | Aminotransferase | |
| DapD | Tetrahydrodipicolinate | |
| | N-Succinyltransferase | |
| DapE | N-succinyl-LL- | |
| | diaminopimelate | |
| | Desuccinylase | |
| DapF | Diaminopimelate | |
| | Epimerase | |
| LysA | Diaminopimelate | Last step in lysine biosynthesis |
| | Decarboxylase | |
| Ddh | Diaminopimelate | Redundant one-step pathway for |
| | Dehydrogenase | converting tetrahydrodipicolinate |
| | | to meso-diaminopimelate in |
| | | Corynebacteria |

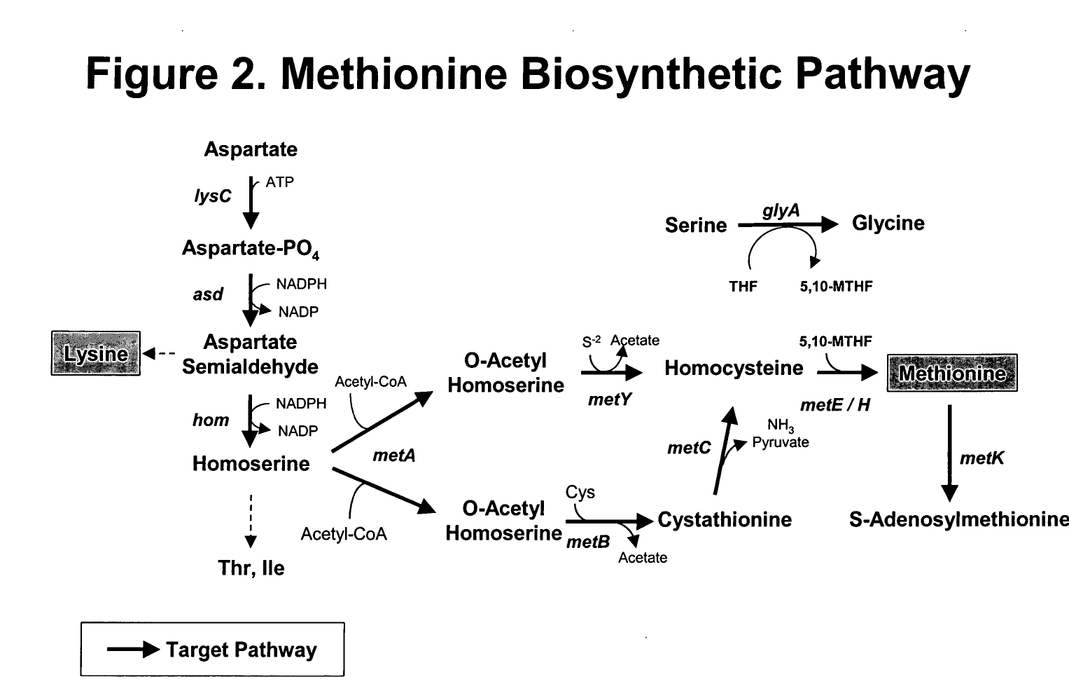

Steps in the biosynthesis of methionine are diagrammed in FIG. 2. Examples of enzymes that regulate methionine biosynthesis include: Homoserine dehydrogenase (Hom), O-homoserine acetyltransferase (MetA), and O-acetylhomoserine sulfhydrylase (MetY). Overexpression (by increasing copy number of the gene of interest and/or through the use of strong promoters) and/or deregulation of each of these enzymes can enhance production of methionine.

Methionine adenosyltransferase (MetK) catalyzes the production of S-adenosyl-L-methionine from methionine. Reduction of metK-expressed enzyme activity can prevent the conversion of methionine to S-adenosyl-L-methionine, thus enhancing the yield of methionine from bacterial strains. Conversely, if one wanted to enhance carbon flow from methionine to S-adenosyl-L-methionine, the metK gene could be overexpressed or desensitized to feedback inhibition.

Bacterial Host Strains

Suitable host species for the production of amino acids include bacteria of the family Enterobacteriaceae such as an Escherichia coli bacteria and strains of the genus Corynebacterium. The list below contains examples of species and strains that can be used as host strains for the expression of heterologous genes and the production of amino acids.
Escherichia coli W3110 F⁻ IN(rrnD-rrnE)1 λ⁻ (E. coli Genetic Stock Center)
Corynebacterium glutamicum ATCC (American Type Culture Collection) 13032
Corynebacterium glutamicum ATCC 21526
Corynebacterium glutamicum ATCC 21543
Corynebacterium glutamicum ATCC 21608
Corynebacterium acetoglutamicum ATCC 15806
Corynebacterium acetoglutamicum ATCC 21491
Corynebacterium acetoglutamicum NRRL B-11473
Corynebacterium acetoglutamicum NRRL B-11475
Corynebacterium acetoacidophilum ATCC 13870
Corynebacterium melassecola ATCC 17965
Corynebacterium thermoaminogenes FERM BP-1539
Brevibacterium lactis
Brevibacterium lactofermentum ATCC 13869
Brevibacterium lactofermentum NRRL B-1 1470
Brevibacterium lactofermentum NRRL B-1 1471
Brevibacterium lactofermentum ATCC 21799
Brevibacterium lactofermentum ATCC 31269
Brevibacterium flavum ATCC 14067
Brevibacterium flavum ATCC 21269
Brevibacterium flavum NRRL B-11472
Brevibacterium flavum NRRL B-11474
Brevibacterium flavum ATCC 21475
Brevibacterium divaricatum ATCC 14020
   Bacteria Strain for Use a Source of Useful Gene

Suitable species and strains for heterologous bacterial genes include, but are not limited to, these listed below.
Mycobacterium smegmatis ATCC 700084
Amycolatopsis mediterranei
Streptomyces coelicolor A3(2)
Thermobifida fusca ATCC 27730
Erwinia chrysanthemi ATCC 11663
Shewanella oneidensis
Mycobacterium leprae
Mycobacterium tuberculosis H37Rv
Lactobacillus plantarum ATCC 8014
Bacillus sphaericus

Amino acid sequences of exemplary proteins, which can be used to enhance amino acid production, are provided in Table 16. Nucleotide sequences encoding these proteins are provided in Table 17. The sequences that can be expressed in a host strain are not limited to those sequences provided by the Tables.

Aspartokinases

Aspartokinases (also referred to as aspartate kinases) are enzymes that catalyze the first committed step in the biosynthesis of aspartic acid family amino acids. The level and activity of aspartokinases are typically regulated by one or more end products of the pathway (lysine or lysine plus threonine depending upon the bacterial species), both through feedback inhibition (also referred to as allosteric regulation) and transcriptional control (also called repression). Bacterial homologs of coryneform and E. coli aspartokinases can be used to enhance amino acid production. Coryneform and E. coli aspartokinases can be expressed in heterologous organisms to enhance amino acid production.

Homologs of the LysCprotein from Coryneform bacteria

In Coryneform bacteria, aspartokinase is encoded by the lysC locus. The lysC locus contains two overlapping genes, lysC alpha and lysC beta. LysC alpha and lysC beta code for the 47- and 18-kD subunits of aspartokinase, respectively. A third open-reading frame is adjacent to the lysC locus, and encodes aspartate semialdehyde dehydrogenase (asd). The asd start codon begins 24 base-pairs downstream from the end of the lysC open-reading frame, is expressed as part of the lysC operon.

The primary sequence of aspartokinase proteins and the structure of the lysC loci are conserved across several members of the order Actinomycetales. Examples of organisms that encode both an aspartokinase and an aspartate semialdehyde dehydrogenase that are highly related to the proteins from coryneform bacteria include Mycobacterium smegmatis, Amycolatopsis mediterranei, Streptomyces coelicolor A3(2), and Thermobifida fusca. In some instances these organisms contain the lysC and asd genes arranged as in coryneform bacteria. Table 2 displays the percent identity of proteins from these Actinomycetes to the C. glutamicum aspartokinase and aspartate semialdehyde dehydrogenase proteins.

TABLE 2
Percent Identity of Heterologous Aspartokinase and Aspartate
Semialdehyde Dehydrogenase Proteins to C. glutamicum Proteins

| | Aspartokinase | Aspartate Semialdehyde |
|---|---|---|
| | (% Identity to | Dehydrogenase (% Identity |
| Organism | C. glutamicum LysC) | to C. glutamicum Asd) |
| Mycobacterium | 73 | 68 |
| smegmatis | | |
| Amycolatopsis | 73 | 62 |
| mediterranei | | |
| Streptomyces | 64 | 50 |
| coelicolor | | |
| Thermobifida | 64 | 48 |
| fusca | | |

Isolates of source strains such as Mycobacterium smegmatis, Amycolatopsis mediterranei, Streptomyces coelicolor, and Thermobifida fusca are available. The lysC operons can be amplified from genomic DNA prepared from each source strain, and the resulting PCR product can be ligated into an E. coli/C. glutamicum shuttle vector. The homolog of the aspartokinase enzyme from the source strain can then be introduced into a host strain and expressed.

E. coli Aspartokinase III Homologs

In coryneform bacteria there is concerted feedback inhibition of aspartokinase by lysine and threonine. This is in contrast to E. coli, where there are three distinct aspartokinases that are independently allosterically regulated by lysine, threonine, or methionine. Homologs of the E. coli aspartokinase III (and other isoenzymes) can be used as an alternative source of deregulated aspartokinase proteins. Expression of these enzymes in coryneform bacteria may decrease the complexity of pathway regulation. For example, the aspartokinase III genes are feedback-inhibited only by lysine instead of lysine and threonine. Therefore, the advantages of expressing feedback-resistant alleles of aspartokinase III alleles include: (1) the increased likelihood of complete deregulation; and (2) the possible removal of the need for constructing either “leaky” mutations in hom or threonine auxotrophs that need to be supplemented. These features can result in decreased feedback inhibition by lysine.

Genes encoding aspartokinase III isoenzymes can be isolated from bacteria that are more distantly related to Corynebacteria than the Actinomycetes described above. For example, the E. chysanthemi and S. oneidensis gene products are 77% and 60% identical to the E. coli lysC protein, respectively (and 26% and 35% identical to C. glutamicum LysC). The genes coding for aspartokinase III, or functional variants therof, from the non-Escherichia bacteria, Erwinia chrysanthemi and Shewanella oneidensis can be amplified and ligated into the appropriate shuttle vector for expression in C. glutamicum.

Construction of Deregulated Aspartokinase Alleles

Lysine analogs (e.g. S-(2-aminoethyl)cysteine (AEC)) or high concentrations of lysine (and/or threonine) can be used to identify strains with enhanced production of lysine. A significant portion of the known lysine-resistant strains from both C. glutamicum and E. coli contain mutations at the lysC locus. Importantly, specific amino acid substitutions that confer increased resistance to AEC have been identified, and these substitutions map to well-conserved residues. Specific amino acid substitutions that result in increased lysine productivity, at least in wild-type strains, include, but are not limited to, those listed in Table 3. In many instances, several useful substitutions have been identified at a particular residue. Furthermore, in various examples, strains have been identified that contain more than one lysC mutation. Sequence alignment confirms that the residues previously associated with feedback-resistance (i.e. AEC-resistance) are conserved in a variety of aspartokinase proteins from distantly related bacteria.

TABLE 3
Amino Acid Substitutions That Release
Aspartokinase Feedback Inhibition.

| | Amino Acid |
|---|---|
| Organism | Substitution |
| Corynebacterium glutamicum (or related species) | Ala 279 [symbol] Pro |
| ″ | Ser 301 [symbol] Tyr |
| ″ | Thr 311 [symbol] Ile |
| ″ | Gly 345 [symbol] Asp |
| Escherichia coli (many substitutions identified | Gly 323 [symbol] Asp |
| between amino acids 318-325 and 345-352) | |
| Escherichia coli (many substitutions identified | Leu 325 [symbol] Phe |
| between amino acids 318-325 and 345-352) | |
| Escherichia coli (many substitutions identified | Ser 345 [symbol] Ile |
| between amino acids 318-325 and 345-352) | |
| Escherichia coli (many substitutions identified | Val 347 [symbol] Met |
| between amino acids 318-325 and 345-352) | |

Standard site-directed mutagenesis techniques can be used to construct aspartokinase variants that are not subject to allosteric regulation. After cloning PCR-amplified lysC or aspartokinase III genes into appropriate shuttle vectors, oligonucleotide-mediated site-directed mutagenesis is use to provide modified alleles that encode substitutions such as those listed in Table 3. Vectors containing either wild-type genes or modified alleles can be be transformed into C. glutamicum alongside control vectors. The resulting transformants can be screened, for example, for lysine productivity, increased resistance to AEC, relative cross-feeding of lysine auxotrophs, or other methods known to those skilled in the art to identify the mutant alleles of most interest. Assays to measure lysine productivity and/or enzyme activity can be used to confirm the screening results and select useful mutant alleles. Techniques such as high pressure liquid chromatography (HPLC) and HPLC-mass spectrometry (MS) assays to quantify levels of members of the aspartic acid family of amino acids and related metabolites are known to those skilled in the art.

Methods for random generating amino acid substitutions within the lysC coding sequence, through methods such as mutagenenic PCR, can be used. These methods are familiar to those skilled in the art; for example, PCR can be performed using the GeneMorph PCR mutagenesis kit (Stratagene, La Jolla, Calif.) according to manufacturer's instructions to achieve medium and high range mutation frequencies.

Evaluation of the heterologous enzymes can be carried out in the presence of the LysC, DapA, Pyc, and Ppc proteins that are endogenous to the host strain. In certain instances, it will be helpful to have reagents to specifically assess the functionality of the heterologous biosynthetic proteins. Phenotypic assays for AEC resistance or enzyme assays can be used to confirm function of wild-type and modified variants of heterologous aspartokinases. The function of cloned heterologous genes can be confirmed by complementation of genetically characterized mutants of E. coli or C. glutamicum. Many of the E. coli strains are publicly available from the E. coli Genetic Stock Center (http://cgsc.biology.yale.edu/top.html). C. glutamicum mutants have also been described.

Dihydrodipicolinate Synthases

Dihydrodipicolinate synthase, encoded by dapa, is the branch point enzyme that commits carbon to lysine biosynthesis rather than threonine/methionine production. DapA converts aspartate-β-semialdehyde to 2,3-dihydrodipicolinate. DapA overexpression has been shown to result in increased lysine production in both E. coli and coryneform bacteria. In E. coli, DapA is allosterically regulated by lysine, whereas existing evidence suggests that C. glutamicum regulation occurs at the level of gene expression. Dihydrodipicolinate synthase proteins are not as well conserved amongst Actinomycetes as compared to LysC proteins.

Both wild-type and deregulated DapA proteins that are homologous to the C. glutamicum protein or the E. coli DapA protein can be expressed to enhance lysine production. Candidate organisms that can be sources of dapa genes are shown in Table 4. The known sequence from M. tuberculosis or M. ieprae can be used to identify homologous genes from M. smegmatis.

TABLE 4
Percent Identity of Dihydrodipicolinate Synthase Proteins.

| | % Identity to | % Identity to |
|---|---|---|
| Organism | C. glutamicum DapA | E. coli DapA |
| Corynebacterium glutamicum | 100 | 34 |
| Mycobacterium tuberculosis | 59 | 33 |
| H37Rv * | | |
| Streptomyces coelicolor | 53 | 33 |
| Thermobifida fusca | 48 | 33 |
| Erwinia chrysanthemi | 34 | 81 |

* Can be used for cloning of the M. smegmatis dapA gene.

Amino acid substitutions that relieve feedback inhibition of E. coli DapA by lysine have been described. Examples of such substitutions are listed in Table 5. Some of the residues that can be altered to relieve feedback inhibition are conserved in all of the candidate DapA proteins (e.g. Leu 88, His 118). This sequence conservation suggests that similar substitutions in the proteins from Actinomycetes may further enhance protein function. Site-directed mutagenesis can be employed to engineer deregulated DapA variants.

DapA isolates can be tested for increased lysine production using methods described above. For instance, one could distribute a culture of a lysine-requiring bacterium on a growth medium lacking lysine. A population of dapA mutants obtained by site-directed mutagenesis could then be introduced (through transformation or conjugation) into a wild-type coryneform strain, and subsequently spread onto the agar plate containing the distributed lysine auxotroph. A feedback-resistant dapA mutant would overproduce lysine which would be excreted into the growth medium and satisfy the growth requirement of the auxotroph previously distributed on the agar plate. Therefore a halo of growth of the lysine auxotroph around a dapa mutation-containing colony would indicate the presence of the desired feedback-resistant mutation.

TABLE 5
Amino Acid Substitutions in Dihydrodipicolinate
Synthase That Release Feedback Inhibition.

| | Amino Acid Substitution |
|---|---|
| | (using E. coli DapA amino |
| Organism | acid # as reference |
| Glycine max | Asn 80 [symbol] Ile |
| Nicotiana sylvestris | |
| Escherichia coli | Ala 81 [symbol] Val |
| Zea mays | Glu 84 [symbol] Lys |
| Methylobacillus glycogens | Leu 88 [symbol] Phe |
| Escherichia coli | His 118 [symbol] Tyr |

Pyruvate and Phosphoenolpyruvate Carboxylases

Pyruvate carboxylase (Pyc) and phosphoenolpyruvate carboxylase (Ppc) catalyze the synthesis of oxaloacetic acid (OAA), the citric acid cycle intermediate that feeds directly into lysine biosynthesis. These anaplerotic reactions have been associated with improved yields of several amino acids, including lysine, and are obviously important to maximize OAA formation. In addition, a variant of the C. glutamicum Pyc protein containing a P458S substitution, has been shown to have increased activity, as demonstrated by increased lysine production. Proline 458 is a highly conserved amino acid position across a broad range of pyruvate carboxylases, including proteins from the Actinomycetes S. coelicolor (amino acid residue 449) and M. smegmatis (amino acid residue 448). Similar amino acid substitutions in these proteins may enhance anaplerotic activity. A third gene, PEP carboxykinase (pck), expresses an enzyme that catalyzes the formation of phosphoenolpyruvate from OAA (for gluconeogenesis), and thus functionally competes with pyc and ppc. Enhancing expression ofpyc and ppc can maximize OAA formation. Reducing or eliminatingpck activity can also improve OAA formation.

Homoserine Dehydrogenase

Homoserine dehydrogenase (Hom) catalyzes the conversion of aspartate semialdehyde to homoserine. Hom is feedback-inhibited by threonine and repressed by methionine in coryneform bacteria. It is thought that this enzyme has greater affinity for aspartate semialdehyde than does the competing dihydrodipicolinate synthase (DapA) reaction in the lysine branch, but slight carbon “spillage” down the threonine pathway may still block Hom activity. Feedback-resistant variants of Hom, overexpression of hom, and/or deregulated transcription of hom, or a combination of any of these approaches, can enhance methionine, threonine, isoleucine, or S-adenosyl-L-methionine production. Decreased Hom activity can enhance lysine production. Bifunctional enzymes with homoserine dehydrogenase activity, such as enzymes encoded by E. coli metL (aspartokinase II-homoserine dehydrogenase II) and thrA (aspartokinase 1-homoserine dehydrogenase I), can also be used to enhance amino acid production.

Targeted amino acid substitutions can be generated either to decrease, but not eliminate, Hom activity or to relieve Hom from feedback inhibition by threonine. Mutations that result in decreased Hom activity are referred to as “leaky” Hom mutations. In the C. glutamicum homoserine dehydrogenase, amino acid residues have been identified that can be mutated to either enhance or decrease Hom activity. Several of these specific amino acids are well-conserved in Hom proteins in other Actinomycetes (see Table 6).

TABLE 6
Amino acid substitutions that result in either “leaky” Hom alleles
or Hom proteins relieved of feedback inhibition by threonine.

| C. | Corresponding amino acid residue from | | |
|---|---|---|---|
| glutamicum | heterologous homoserine dehydrogenase | | |
| residue | M. smegmatis | S. coelicolor | T. fusca |
| Leaky Hom | | | |
| alleles | | | |
| L23F | V10 | L10 | L192 |
| V59A | V46 | V46 | V228 |
| V104I | I90 | I91 | I274 |
| Deregulated | | | |
| Hom alleles | | | |
| G378E | G364 | G362 | G545 |
| K428 | N/a | R412 truncation | R595 truncation |
| truncation | | | |
| hom^{dr}* | N/a | R412 (delete bp | R595 (delete bp |
| | | 1937 → frameshift | 1785 → frameshift |
| | | mutation) | mutation) |

*The hom^{dr} mutation is described on page 11 of WO 93/09225. This mutation is a single base pair deletion at 1964 bp that disrupts the hom^{dr}reading frame at codon 429. This results in a frame shift mutation that induces approximately ten amino acid changes and a premature termination, or truncation, i.e., deletion of approximately the last seven amino acid residues of the polypeptide.

It is believed that this single base deletion in the carboxy terminus of the hom dr gene radically alters the protein sequence of the carboxyl terminus of the enzyme, changing its conformation in such a way that the interaction of threonine with a binding site is prevented.

Homoserine O-Acetyltransferase

Homoserine O-acetyltransferase (MetA) acts at the first committed step in methionine biosynthesis (Park, S. et al., Mol. Cells 8:286-294, 1998). The MetA enzyme catalyzes the conversion of homoserine to O-acetyl-homoserine. MetA is strongly regulated by end products of the methionine biosynthetic pathway. In E. coli, allosteric regulation occurs by both S-AM and methionine, apparently at two separate allosteric sites. Moreover, MetJ and S-AM cause transcriptional repression of metA. In coryneform bacteria, MetA may be allosterically inhibited by methionine and S-AM, similarly to E. coli. MetA synthesis can be repressed by methionine alone. In addition, trifluoromethionine-resistance has been associated with metA in early studies. Reduction of negative regulation by S-AM and methionine can enhance methionine or S-adenosyl-L-methionine production. Increased MetA activity can enhance production of aspartate-derived amino acids such as methionine and S-AM, whereas decreased MetA activity can promote the formation of amino acids such as threonine and isoleucine.

O-Acetylhomoserine Sulfhydrylase

O-Acetylhomoserine sulfhydrylase (MetY) catalyzes the conversion of O-acetyl homoserine to homocysteine. MetY may be repressed by methionine in coryneform bacteria, with a 99% reduction in enzyme activity in the presence of 0.5 mM methionine. It is likely that this inhibition represents the combined effect of allosteric regulation and repression of gene expression. In addition, enzyme activity is inhibited by methionine, homoserine, and O-acetylserine. It is possible that S-AM also modulates MetY activity. Deregulated MetY can enhance methionine or S-AM production.

Homoserine Kinase

Homoserine kinase is encoded by thrB gene, which is part of the hom-thrB operon. ThrB phosphorylates homoserine. Threonine inhibition of homoserine kinase has been observed in several species. Some studies suggest that phosphorylation of homoserine by homoserine kinase may limit threonine biosynthesis under some conditions. Increased ThrB activity can enhance production of aspartate-derived amino acids such as isoleucine and threonine, whereas decreased ThrB activity can promote the formation of amino acids including, but not limited to, lysine and methionine.

Methionine Adenosyltransferase

Methionine adenosyltransferase converts methionine to S-adenosyl-L-methionine (S-AM). Down-regulating methionine adenosyltransferase (MetK) can enhance production of methionine by inhibiting conversion to S-AM. Enhancing expression of metK or activity of MetK can maximize production of S-AM.

O-Succinylhomoserine (thio)-lyase/O-acetylhomoserine (thio)-lyase O-Succinylhomoserine (thio)-lyase (MetB; also known as cystathionine gamma-synthase) catalyzes the conversion of O-succinyl homoserine or O-acetyl homoserine to cystathionine. Increasing expression or activity of MetB can lead to increased methionine or S-AM.

Cystathionine Beta-Lyase

Cystathionine beta-lyase (MetC) can convert cystathionine to homocysteine. Increasing production of homocysteine can lead to increased production of methionine. Thus, increased MetC expression or activity can increase methionine or S-adenosyl-L-methionine production.

Glutamate Dehydrogenase

The enzyme glutamate dehydrogenase, encoded by the gdh gene, catalyses the reductive amination of α-ketoglutarate to yield glutamic acid. Increasing expression or activity of glutamate dehydrogenase can lead to increased lysine, threonine, isoleucine, valine, proline, or tryptophan.

Diaminopimelate Dehydrogenase

Diaminopimelate dehydrogenase, encoded by the ddh gene in coryneform bacteria, catalyzes the the NADPH-dependent reduction of ammonia and L-2-amino-6-oxopimelate to form meso-2,6-diaminopimelate, the direct precursor of L-lysine in the alternative pathway of lysine biosynthesis. Overexpression of diaminopimelate dehydrogenase can increase lysine production.

Detergent Sensitivity Rescuer

Detergent sensitivity rescuer (dtsR1), encoding a protein related to the alpha subunit of acetyl CoA carboxylase, is a surfactant resistance gene. Increasing expression or activity of DtsR1 can lead to increased production of lysine.

5-Methyltetrahydrofolate Homocysteine Methyltransferase

5-Methyltetrahydrofolate homocysteine methyltransferase (MetH) catalyzes the conversion of homocysteine to methionine. This reaction is dependent on cobalamin (vitamin B12). Increasing MetH expression or activity can lead to increased production of methionine or S-adenosyl-L-methionine.

5-Methyltetrahydropteroyltriglutamate-homocysteine Methyltransferase

5-Methyltetrahydropteroyltriglutamate-homocysteine methyltransferase (MetE) also catalyzes the conversion of homocysteine to methionine. Increasing MetE expression or activity can lead to increased production of methionine or S-adenosyl-L-methionine.

Serine Hydroxymethyltransferase

Increasing serine hydroxymethyltransferase (GlyA) expression or activity can lead to enhanced methionine or S-adenosyl-L-methionine production.

5,10-Methylenetetrahydrofolate Reductase

5,10-Methylenetetrahydrofolate reductase (MetF) catalyzes the reduction of methylenetetrahydrofolate to methyltetrahydrofolate, a cofactor for homocysteine methylation to methionine. Increasing expression or activity of MetF can lead to increased methionine or S-adenosyl-L-methionine production.

Serine O-acetyltransferase

Serine O-acetyltransferase (CysE) catalyzes the conversion of serine to O-acetylserine. Increasing expression or activity of CysE can lead to increased expression of methionine or S-adenosyl-L-methionine.

D-3-phosphoglycerate Dehydrogenase

D-3-phosphoglycerate dehydrogenase (SerA) catalyzes the first step in serine biosynthesis, and is allosterically inhibited by serine. Increasing expression or activity of SerA can lead to increased production of methionine or S-adenosyl-L-methionine.

McbR Gene Product

The mcbR gene product of C. glutamicum was identified as a putative transcriptional repressor of the TetR-family and may be involved in the regulation of the metabolic network directing the synthesis of methionine in C. glutamicum (Rey et al., J. Biotechnol. 103(1):51-65, 2003). The mcbR gene product represses expression of metY, metK, cysK, cysl, hom, pyk, ssuD, and possibly other genes. It is possible that McbR represses expression in combination with small molecules such as S-AM or methionine. To date, specific alleles of McbR that prevent binding of either S-AM or methionine have not been identified. Reducing expression of McbR, and/or preventing regulation of McbR by S-AM can enhance amino acid production.

McbR is involved in the regulation of sulfur containing amino acids (e.g., cysteine, methionine). Reduced McbR expression or activity can also enhance production of any of the aspartate family of amino acids that are derived from homoserine (e.g., homoserine, O-acetyl-L-homoserine, O-succinyl-L-homoserine, cystathionine, L-homocysteine, L-methionine, S-adenosyl-L-methionine (S-AM), O-phospho-L-homoserine, threonine, 2-oxobutanoate, (S)-2-aceto-2-hydroxybutanoate, (S)-2-hydroxy-3-methyl-3-oxopentanoate, (R)-2,3-Dihydroxy-3-methylpentanoate, (R)-2-oxo-3-methylpentanoate, and L-isoleucine).

Lysine Exporter Protein

Lysine exporter protein (LysE) is a specific lysine translocator that mediates efflux of lysine from the cell. In C. glutamicum with a deletion in the lysE gene, L-lysine can reach an intracellular concentration of more than 1M. (Erdmann, A., et al. J. Gen Microbiol. 139,:3115-3122, 1993). Overexpression or increased activity of this exporter protein can enhance lysine production.

Efflux Proteins

A substantial number of bacterial genes encode membrane transport proteins. A subset of these membrane transport protein mediate efflux of amino acids from the cell. For example, Corynebacterium glutamicum express a threonine efflux protein. Loss of activity of this protein leads to a high intracellular accumulation of threonine (Simic et al., J. Bacteriol. 183(18):5317-5324, 2001). Increasing expression or activity of efflux proteins can lead to increased production of various amino acids. Useful efflux proteins include proteins of the drug/metabolite transporter family. The C. glutamicum proteins listed in Table 16 or homologs thereof can be used to increase amino acid production.

Isolation of Bacterial Genes

Bacterial genes for expression in host strains can be isolated by methods known in the art. See, for example, Sambrook, J., and Russell, D. W. (Molecular Cloning: A Laboratory Manual, 3nd Ed., Cold Spring Harbor Laboratory Press, Cold Spring Harbor, N.Y., 2001) for methods of construction of recombinant nucleic acids. Genomic DNA from source strains can be prepared using known methods (see, e.g., Saito, H. and, Miura, K. Biochim Biophys Acta. 72:619-629, 1963) and genes can be amplified from genomic DNA using PCR (U.S. Pats. 4,683,195 and 4,683,202, Saiki, et al. Science 230:350-1354, 1985).

DNA primers to be used for the amplification reaction are those complemental to both 3′-terminals of a double stranded DNA containing an entire region or a partial region of a gene of interest. When only a partial region of a gene is amplified, it is necessary to use such DNA fragments as primers to perform screening of a DNA fragment containing the entire region from a chromosomal DNA library. When the entire region gene is amplified, a PCR reaction solution including DNA fragments containing the amplified gene is subjected to agarose gel electrophoresis, and then a DNA fragment is extracted and cloned into a vector appropriate for expression in bacterial systems.

DNA primers for PCR may be adequately prepared on the basis of, for example, a sequence known in the source strain (Richaud, F. et al., J. Bacteriol. 297,1986). For example, primers that can amplify a region comprising the nucleotide bases coding for the heterologous gene of interest can be used. Synthesis of the primers can be performed by an ordinary method such as a phosphoamidite method (see Tetrahed Lett. 22:1859,1981) by using a commercially available DNA synthesizer (for example, DNA Synthesizer Model 380B produced by Applied Biosystems Inc.). Further, the PCR can be performed by using a commercially available PCR apparatus and Taq DNA polymerase, or other polymerases that display higher fidelity, in accordance with a method designated by the supplier.

Construction of Variant Alleles

Many enzymes that regulate amino acid production are subject to allosteric feedback inhibition by biosynthetic pathway intermediates or end products. Useful variants of these enzymes can be generated by substitution of residues responsible for feedback inhibition. For example, enzymes such as homoserine O-acetyltransferase (encoded by metA) are feedback-inhibited by S-AM. To generate deregulated variants of homoserine O-acetyltransferase, we identified putative S-AM binding residues within the amino acid sequence of homoserine O-acetyltransferase, and then constructed plasmids to express MetA variants containing specific amino acid substitutions that are predicted to confer increased resistance to allosteric regulation by S-AM. Strains expressing these variants showed increased production of methionine (see Examples, below).

Additional putative S-AM binding residues in various enzymes include, but are not limited to, those listed in Tables 9 and 10. One or more of the residues in Tables 9 and 10 can be substituted with a non-conservative residue, or with an alanine (e.g., where the wild type residue is other than an alanine). Sequence alignment confirms that the residues potentially associated with feedback-sensitivity to S-AM are conserved in a variety of MetA and MetY proteins from distantly related bacteria.

Standard site-directed mutagenesis techniques can be used to construct variants that are less sensitive to allosteric regulation. After cloning a PCR-amplified gene or genes into appropriate shuttle vectors, oligonucleotide-mediated site-directed mutagenesis is use to provide modified alleles that encode specific amino acid substitutions. Vectors containing either wild-type genes or modified alleles can be transformed into C. glutamicum, or another suitable host strain, alongside control vectors. The resulting transformants can be screened, for example, for amino acid productivity, increased resistance to feedback inhibition by S-AM, activity of the enzyme of interest, or other methods known to those skilled in the art to identify the variant alleles of most interest. Assays to measure amino acid productivity and/or enzyme activity can be used to confirm the screening results and select useful variant alleles. Techniques such as high pressure liquid chromatography (HPLC) and HPLC-mass spectrometry (MS) assays to quantify levels of amino acids and related metabolites are known to those skilled in the art.

Methods for generating random amino acid substitutions within a coding sequence, through methods such as mutagenenic PCR, can be used (e.g., to generate variants for screening for reduced feedback inhibition, or for introducing further variation into enhanced variant sequences). For example, PCR can be performed using the GeneMorph® PCR mutagenesis kit (Stratagene, La Jolla, Calif.) according to manufacturer's instructions to achieve medium and high range mutation frequencies. Other methods are also known in the art.

Evaluation of enzymes can be carried out in the presence of additional enzymes that are endogenous to the host strain. In certain instances, it will be helpful to have reagents to specifically assess the functionality of a biosynthetic protein that is not endogenous to the organism (e.g., an episomally expressed protein). Phenotypic assays for feedback inhibition or enzyme assays can be used to confirm function of wild-type and variants of biosynthetic enzymes. The function of cloned genes can be confirmed by complementation of genetically characterized mutants of the host organism (e.g., the host E. coli or C. glutamicum bacterium). Many of the E. coli strains are publicly available from the E. coli Genetic Stock Center (http://cgsc.biology.yale.edu/top.html). C. glutamicum mutants have also been described.

Expression of Genes

Bacterial genes can be expressed in host bacterial strains using methods known in the art. In some cases, overexpression of a bacterial gene (e.g., a heterologous and/or variant gene) will enhance amino acid production by the host strain. Overexpression of a gene can be achieved in a variety of ways. For example, multiple copies of the gene can be expressed, or the promoter, regulatory elements, and/or ribosome binding site upstream of a gene (e.g., a variant allele of a gene, or an endogenous gene) can be modified for optimal expression in the host strain. In addition, the presence of even one additional copy of the gene can achieve increased expression, even where the host strain already harbors one or more copies of the corresponding gene native to the host species. The gene can be operably linked to a strong constitutive promoter or an inducible promoter (e.g., trc, lac) and induced under conditions that facilitate maximal amino acid production. Methods to enhance stability of the mRNA are known to those skilled in the art and can be used to ensure consistently high levels of expressed proteins. See, for example, Keasling, J., Trends in Biotechnology 17:452-460, 1999. Optimization of media and culture conditions may also enhance expression of the gene.

Methods for facilitating expression of genes in bacteria have been described. See, for example, Guerrero, C, et al., Gene 138(1-2):35-41, 1994; Eikmanns, B. J., et al. Gene 102(1):93-8, 1991; Schwarzer, A., and Puhler, A. Biotechnol. 9(1):84-7, 1991; Labarre, J., et al., J Bacteriol. 175(4):1001-7, 1993; Malumbres, M., et al. Gene 134(1):15-24, 1993; Jensen, P. R., and Hammer, K. Biotechnol Bioeng. 158(2-3):191-5, 1998; Makrides, S. C. Microbiol Rev. 60(3):512-38, 1996; Tsuchiya et al. Bio/Technology 6:428-431,1988; U.S. Pat. No. 5,965,931; U.S. Pat. No. 4,601,893; and U.S. Pat. No. 5,175,108.

A gene of interest (e.g., a heterologous or variant gene) should be operably linked to an appropriate promoter, such as a native or host strain-derived promoter, a phage promoter, one of the well-characterized E. coli promoters (e.g. tac, trp, phoA, araBAD, or variants thereof etc.). Other suitable promoters are also available. In one embodiment, the heterologous gene is operably linked to a promoter that permits expression of the heterologous gene at levels at least 2-fold, 5-fold, or 10-fold higher than levels of the endogenous homolog in the host strain. Plasmid vectors that aid the process of gene amplification by integration into the chromosome can be used. See, for example, by Reinscheid et al. (Appl. Environ Microbiol. 60: 126-132,1994). In this method, the complete gene is cloned in a plasmid vector that can replicate in a host (typically E. coli), but not in C. glutamicum. These vectors include, for example, pSUP301 (Simon et al., Bio/Technol. 1, 784-79,1983), pK18mob or pK19mob (Schfer et al., Gene 145:69-73, 1994), PGEM-T (Promega Corp., Madison, Wis., USA), pCR2.1 -TOPO (Shuman J Biol Chem. 269:32678-84, 1994; U.S. Pat. No. 5,487,993), pCR.RTM.Blunt (Invitrogen, Groningen, Holland; Bernard et al., J Mol Biol., 234:534-541,1993), pEMI (Schrumpf et al. J Bacteriol. 173:4510-4516, 1991) or pBGS8 (Spratt et al., Gene 41:337-342, 1996). The plasmid vector that contains the gene to be amplified is then transferred into the desired strain of C. glutamicum by conjugation or transformation. The method of conjugation is described, for example, by Schfer et al. (Appl Environ Microbiol. 60:756-759,1994). Methods for transformation are described, for example, by Thierbach et al. (Appl Microbiol Biotechnol. 29:356-362,1988), Dunican and Shivnan (Bio/Technol. 7:1067-1070,1989) and Tauch et al. (FEMS Microbiol Lett. 123:343-347,1994). After homologous recombination by means of a genetic cross over event, the resulting strain contains the desired gene integrated in the host genome.

An appropriate expression plasmid can also contain at least one selectable marker. A selectable marker can be a nucleotide sequence that confers antibiotic resistance in a host cell. These selectable markers include ampicillin, cefazolin, augmentin, cefoxitin, ceftazidime, ceftiofur, cephalothin, enrofloxicin, kanamycin, spectinomycin, streptomycin, tetracycline, ticarcillin, tilmicosin, or chloramphenicol resistance genes. Additional selectable markers include genes that can complement nutritional auxotrophies present in a particular host strain (e.g. leucine, alanine, or homoserine auxotrophies).

In one embodiment, a replicative vector is used for expression of the heterologous gene. An exemplary replicative vector can include the following: a) a selectable marker, e.g., an antibiotic marker, such as kanR (from pACYC184); b) an origin of replication in E. coli, such as the P15a ori (from pACYC 184); c) an origin of replication in C. glutamicum such as that found in pBL1; d) a promoter segment, with or without an accompanying repressor gene; and e) a terminator segment. The promoter segment can be a lac, trc, trcRBS, tac, or λP_{L}/λP_{R} (from E. coli), orphoA, gpd, rplM, rpsJ (from C. glutamicum). The repressor gene can be lacIor cI857, for lac, trc, trcRBS, tac and λP_{L}/λP_{R}, respectively. The terminator segment can be from E. coli rrnB (from ptrc99a), the T7 terminator (from pET26), or a terminator segment from C. glutamicum.

In another embodiment, an integrative vector is used for expression of the heterologous gene. An exemplary integrative vector can include: a selectable marker, e.g., an antibiotic marker, such as kanR (from pACYC l 84); b) an origin of replication in E. coli, such as the P15a ori (from pACYC184); c) and d) two segments of the C. glutamicum genome that flank the segment to be replaced, such as the pck or hom genes; e) the sacB gene from B. subtilis; f) a promoter segment to control expression of the heterologous gene, with or without an accompanying repressor gene; and g) a terminator segment. The promoter segment can be lac, trc, trcRBS, tac, or λP_{L}/λP_{R} (from E. coli), or phoa, gpd, rplM, rpsj (from C. glutamicum). The repressor genes can be lacI or cI, for lac, trc, trcRBS, tac and λP_{L}/λP_{R}, respectively. The terminator segment can be from E. coli rrnB (from ptrc99a), the T7 terminator (from pET26), or a terminator segment from C. glutamicum. The possible integrative or replicative plasmids, or reagents used to construct these plasmids, are not limited to those described herein. Other plasmids are familiar to those in the art.

For use of terminator segments from C. glutamicum, the terminator and flanking sequences can be supplied by a single gene segment. In this case, the above elements will be arranged in the following sequence on the plasmid: marker; origin of replication; a segment of the C. glutamicum genome that flanks the segment to be replaced; promoter; C. glutamicum terminator; sacB gene. The sacB gene can also be placed between the origin of replication and the C. glutamicum flanking segment. Integration and excision results in the insertion of only the promoter, terminator, and the gene of interest.

A multiple cloning site can be positioned in one of several possible locations between the plasmid elements described above in order to facilitate insertion of the particular genes of interest (e.g., lysC, etc.) into the plasmid. For both replicative and integrative vectors, the addition of an origin of conjugative transfer, such as RP4 mob, can facilitate gene transfer between E. coli and C. glutamicum.

In one embodiment, a bacterial gene is expressed in a host strain with an episomal plasmid. Suitable plasmids include those that replicate in the chosen host strain, such as a coryneform bacterium. Many known plasmid vectors, such as e.g. pZ1 (Menkel et al., Applied Environ Microbiol. 64:549-554, 1989), pEKEx1 (Eikmanns et al., Gene 102:93-98,1991) or pHS2-1 (Sonnen et al., Gene 107:69-74, 1991) are based on the cryptic plasmids pHM1519, pBL1 or pGA1. Other plasmid vectors that can be used include those based on pCG4 (U.S. Pat. No. 4,489,160), or pNG2 (Serwold-Davis et al., FEMS Microbiol Lett. 66:119-124,1990), or pAG1 (U.S. Pat. No. 5,158,891). Alternatively, the gene or genes may be integrated into chromosome of a host microorganism by a method using transduction, transposon (Berg, D. E. and Berg, C. M., Bio/Technol. 1:417,1983), Mu phage (Japanese Patent Application Laid-open No. 2-109985) or homologous or non-homologous recombination (Experiments in Molecular Genetics, Cold Spring Harbor Lab.,1972).

In addition, it may be advantageous for the production of amino acids to enhance one or more enzymes of the particular biosynthesis pathway, of glycolysis, of anaplerosis, or of amino acid export, using more than one gene or using a gene in combination with other biosynthetic pathway genes.

It also may be advantageous to simultaneously attenuate the expression of particular gene products to maximize production of a particular amino acid. For example, attenuation of metK expression or MetK activity can enhance methionine production by prevention conversion of methionine to S-AM.

Methods of introducing nucleic acids into host cells are known in the art. See, for example, Sambrook, J., and Russell, D. W. Molecular Cloning: A Laboratory Manual, 3^{nd} Ed., Cold Spring Harbor Laboratory Press, Cold Spring Harbor, N.Y., 2001. Suitable methods include transformation using calcium chloride (Mandel, M. and Higa, A. J. Mol Biol. 53:159, 1970) and electroporation (Rest, M. E. van der, et al. Appl Microbiol. Biotechnol. 52:541-545, 1999), or conjugation.

Cultivation of Bacteria

The bacteria containing gene(s) of interest (e.g., heterologous genes, variant genes encoding enzymes with reduced feedback inhibition) can be cultured continuously or by a batch fermentation process (batch culture). Other commercially used process variations known to those skilled in the art include fed batch (feed process) or repeated fed batch process (repetitive feed process). A summary of known culture methods is described in the textbook by Chmiel (Bioprozesstechnik 1. Einfuhrung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)).

The culture medium to be used fulfills the requirements of the particular host strains. General descriptions of culture media suitable for various microorganisms can be found in the book “Manual of Methods for General Bacteriology” of the American Society for Bacteriology (Washington D.C., USA, 1981), although those skilled in the art will recognize that the composition of the culture medium is often modified beyond simple growth requirements in order to maximize product formation.

Sugars and carbohydrates, such as e.g., glucose, sucrose, lactose, fructose, maltose, starch and cellulose; oils and fats, such as e.g. soy oil, sunflower oil, groundnut oil and coconut fat; fatty acids, such as e.g. palmitic acid, stearic acid and linoleic acid; alcohols, such as e.g. glycerol and ethanol; and organic acids, such as e.g. acetic acid, can be used as the source of carbon, either individually or as a mixture.

Organic nitrogen-containing compounds, such as peptones, yeast extract, meat extract, malt extract, corn steep liquor, soy protein hydrolysate, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Phosphoric acid, potassium dihydrogen phosphate, dipotassium hydrogen phosphate, or the corresponding sodium-containing salts can be used as the source of phosphorus.

Organic and inorganic sulfur-containing compounds, such as, for example, sulfates, thiosulfates, sulfites, reduced sources such as H₂S, sulfides, derivatives of sulfides, methyl mercaptan, thioglycolytes, thiocyanates, and thiourea, can be used as sulfur sources for the preparation of sulfur-containing amino acids.

The culture medium can also include salts of metals, e.g., magnesium sulfate or iron sulfate, which are necessary for growth. Essential growth substances, such as amino acids and vitamins (e.g. cobalamin), can be employed in addition to the above-mentioned substances. Suitable precursors can moreover be added to the culture medium. The starting substances mentioned can be added to the culture as a single batch, or can be fed in during the culture at multiple points in time.

Basic compounds, such as sodium hydroxide, potassium hydroxide, calcium carbonate, ammonia or aqueous ammonia, or acid compounds, such as phosphoric acid or sulfuric acid, can be employed in a suitable manner to control the pH. Antifoams, such as e.g. fatty acid polyglycol esters, can be employed to control the development of foam. Suitable substances having a selective action, such as e.g. antibiotics, can be added to the medium to maintain the stability of plasmids. To maintain aerobic conditions, oxygen or oxygen-containing gas mixtures, such as e.g. air, are introduced into the culture. The temperature of the culture is typically between 20-45° C. and preferably 25-40° C. Culturing is continued until a maximum of the desired product has formed, usually within 10 hours to 160 hours.

The fermentation broths obtained in this way, can contain a dry weight of 2.5 to 25 wt. % of the amino acid of interest. It also can be advantageous if the fermentation is conducted in such that the growth and metabolism of the production microorganism is limited by the rate of carbohydrate addtion for some portion of the fermentation cycle, preferably at least for 30% of the duration of the fermentation. For example, the concentration of utilizable sugar in the fermentation medium is maintained at <3 g/l during this period.

The fermentation broth can then be further processed. All or some of the biomass can be removed from the fermentation broth by any solid-liquid separation method, such as centrifugation, filtration, decanting or a combination thereof, or it can be left completely in the broth. Water is then removed from the broth by known methods, such as with the aid of a multiple-effect evaporator, thin film evaporator, falling film evaporator, or by reverse osmosis. The concentrated fermentation broth can then be worked up by methods of freeze drying, spray drying, fluidized bed drying, or by other processes to give a preferably free-flowing, finely divided powder.

The free-flowing, finely divided powder can then in turn by converted by suitable compacting or granulating processes into a coarse-grained, readily free-flowing, storable and largely dust-free product. In the granulation or compacting it can be advantageous to use conventional organic or inorganic auxiliary substances or carriers, such as starch, gelatin, cellulose derivatives or similar substances, such as are conventionally used as binders, gelling agents or thickeners in foodstuffs or feedstuffs processing, or further substances, such as, for example, silicas, silicates or stearates.

Alternatively, however, the product can be absorbed on to an organic or inorganic carrier substance which is known and conventional in feedstuffs processing, for example, silicas, silicates, grits, brans, meals, starches, sugars or others, and/or mixed and stabilized with conventional thickeners or binders.

Finally, the product can be brought into a state in which it is stable to digestion by animal stomachs, in particular the stomach of ruminants, by coating processes using film-forming agents, such as, for example, metal carbonates, silicas, silicates, alginates, stearates, starches, gums and cellulose ethers, as described in DE-C-4100920.

If the biomass is separated off during the process, further inorganic solids, for example, those added during the fermentation, are generally removed.

In one aspect of the invention, the biomass can be separated off to the extent of up to 70%, preferably up to 80%, preferably up to 90%, preferably up to 95%, and particularly preferably up to 100%. In another aspect of the invention, up to 20% of the biomass, preferably up to 15%, preferably up to 10%, preferably up to 5%, particularly preferably no biomass is separated off.

Organic substances which are formed or added and are present in the solution of the fermentation broth can be retained or separated by suitable processes. These organic substances include organic by-products that are optionally produced, in addition to the desired L-amino acid, and optionally discharged by the microorganisms employed in the fermentation. These include L-amino acids chosen from the group consisting of L-lysine, L-valine, L-threonine, L-alanine, L-methionine, L-isoleucine, or L-tryptophan. They include vitamins chosen from the group consisting of vitamin B1 (thiamine), vitamin B2 (riboflavin), vitamin B5 (pantothenic acid), vitamin B6 (pyridoxine), vitamin B12 (cyanocobalamin), nicotinic acid/nicotinanide and vitamin E (tocopherol). They also include organic acids that carry one to three carboxyl groups, such as, acetic acid, lactic acid, citric acid, malic acid or fumaric acid. Finally, they also include sugars, for example, trehalose. These compounds are optionally desired if they improve the nutritional value of the product.

These organic substances, including L- and/or D-amino acid and/or the racemic mixture D,L-amino acid, can also be added, depending on requirements, as a concentrate or pure substance in solid or liquid form during a suitable process step. These organic substances mentioned can be added individually or as mixtures to the resulting or concentrated fermentation broth, or also during the drying or granulation process. It is likewise possible to add an organic substance or a mixture of several organic substances to the fermentation broth and a further organic substance or a further mixture of several organic substances during a later process step, for example granulation. The product described above can be used as a feed additive, i.e. feed additive, for animal nutrition. For methods of preparing amino acids for use as feed additives, see, e.g., WO 02/18613, the contents of which are herein incorporated by reference.

### EXAMPLE 1

### Construction of Vectors for Expression of Genes for Enhancing Production of Aspartate-Derived Amino Acids

Plasmids were generated for expression of genes relevant to the production of aspartate-derived amino acids. Many of the target genes are shown in FIG. 1 and 2, which depicts most of the biosynthetic genes directly involved in producing aspartate-derived amino acids. These plasmids, which may either replicate autonomously or integrate into the host C. glutamicum chromosome, were introduced into strains of corynebacteria by electroporation as described (see Follettie, M. T., et al. J. Bacteriol. 167:695-702, 1993). All plasmids contain the kanR gene that confers resistance to the antibiotic kanamycin. Transformants were selected on media containing kanamycin (25 mg/L).

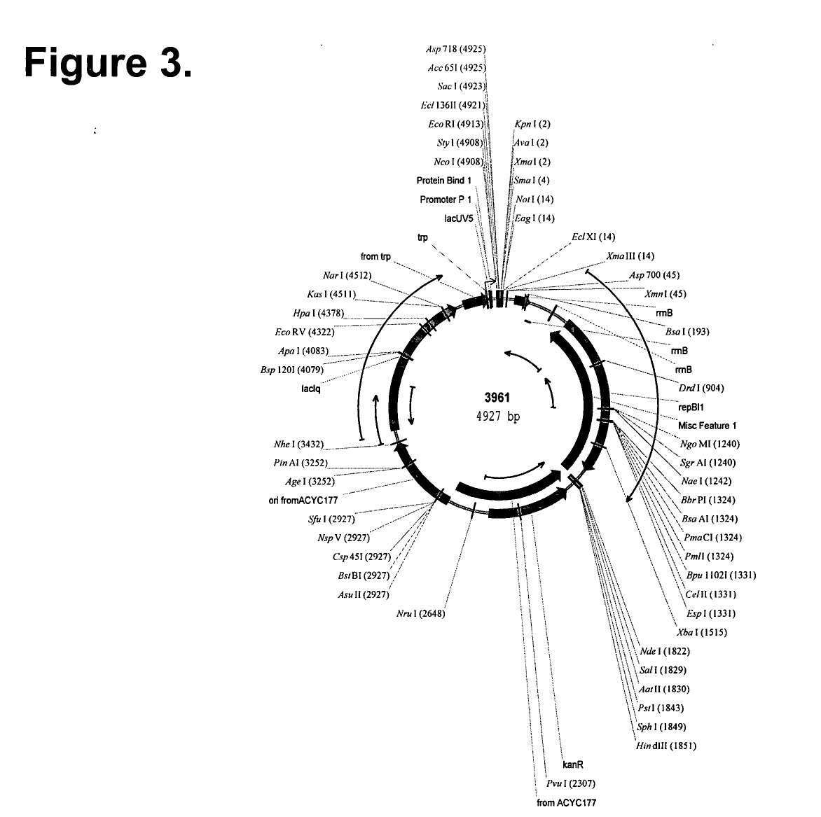

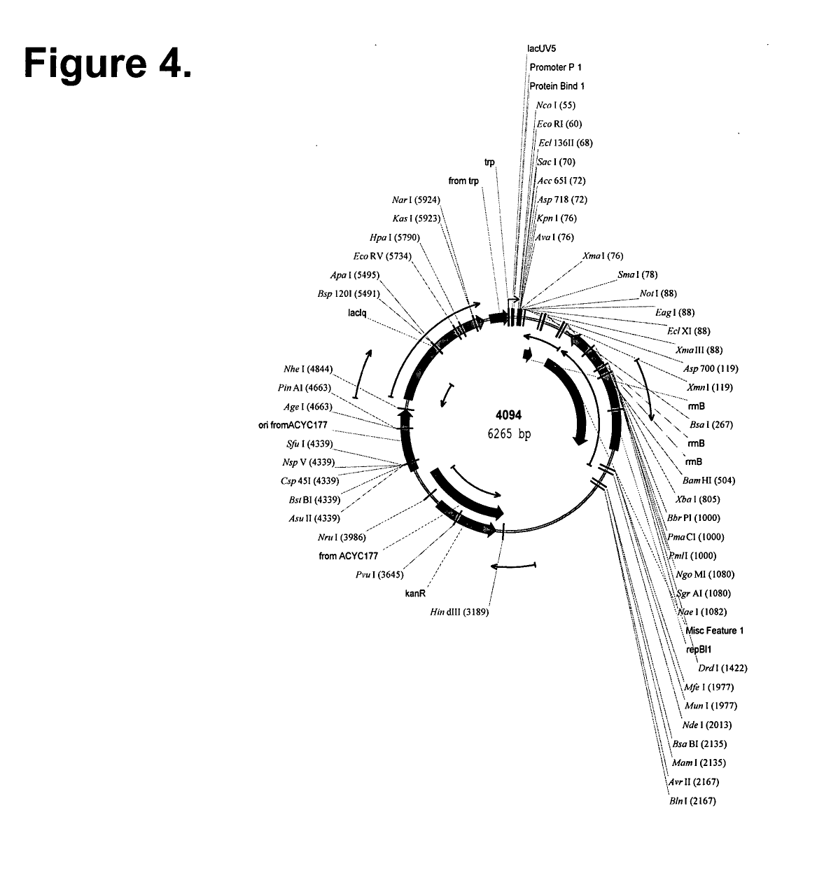

For expression from episomal plasmids, vectors were constructed using derivatives of the cryptic C. glutamicum low-copy pBL1 plasmid (see Santamaria et al. J. Gen. Microbiol. 130:2237-2246, 1984). Episomal plasmids contain sequences that encode a replicase, which enables replication of the plasmid within C. glutamicum; therefore, these plasmids can be propagated without integration into the chromosome. Plasmids MB3961 and MB4094 were the vector backbones used to construct episomal expression plasmids described herein (see FIGS. 3 and 4). Plasmid MB4094 contains an improved origin of replication, relative to MB3961, for use in corynebacteria; therefore, this backbone was used for most studies. Both MB3961 and MB4094 contain regulatory sequences from pTrc99A (see Amann et al., Gene 69:301-315, 1988). The 3′ portion of the lacIq-trc IPTG-inducible promoter cassette resides within the polylinker in such a way that genes of interest can be inserted as fragments containing NcoI-NotI compatible overhangs, with the NcoI site adjacent to the start site of the gene of interest (additional polylinker sites such as KpnI can also be used instead of the NotI site). In addition, useful promoters such as a modified trc promoter (trcRBS) and the C. glutamicum gpd, rplM, and rpsJ promoters can be inserted into the MB3961 and MB4094 backbones on convenient restriction fragments, including NheI-NcoI fragments. The trcRBS promoter contains a modified ribosomal-binding site that was shown to enhance levels of expressed proteins. The sequences of promoters employed in these studies for expression of genes are found in Table 7.

TABLE 7
Promoters used to control expression of genes in corynebacteria.

| | | SEQ ID |
|---|---|---|
| Promoter | Sequence | NO: |
| Laclq-trc | ctagctacgttgacaccatcgaatggtgcaaaacctttcgcggtatggcatgatagcgcccggaa | 297 |
| | gagagtcaattcagggtggtgaatgtgaaaccagtaacgttatacgatgtcgcagagtatgccggt | |
| | gtctcttatcagaccgtttcccgcgtggtgaaccaggccagccacgtttctgcgaaaacgcggga | |
| | aaaagtggaagcggcgatggcggagctgaattacattcccaaccgcgtggcacaacaactggc | |
| | gggcaaacagtcgttgctgattggcgttgccacctccagtctggccctgcacgcgccgtcgcaaa | |
| | ttgtcgcggcgattaaatctcgcgccgatcaactgggtgccagcgtggtggtgtcgatggtagaa | |
| | cgaagcggcgtcgaagcctgtaaagcggcggtgcacaatcttctcgcgcaacgcgtcagtggg | |
| | ctgatcattaactatccgctggatgaccaggatgccattgctgtggaagctgcctgcactaatgttc | |
| | cggcgttatttcttgatgtctctgaccagacacccatcaacagtattattttctcccatgaagacggta | |
| | cgcgactgggcgtggagcatctggtcgcattgggtcaccagcaaatcgcgctgttagcgggccc | |
| | attaagttctgtctcggcgcgtctgcgtctggctggctggcataaatatctcactcgcaatcaaattc | |
| | agccgatagcggaacgggaaggcgactggagtgccatgtccggttttcaacaaaccatgcaaat | |
| | gctgaatgagggcatcgttcccactgcgatgctggttgccaacgatcagatggcgctgggcgca | |
| | atgcgcgccattaccgagtccgggctgcgcgttggtgcggatatctcggtagtgggatacgacga | |
| | taccgaagacagctcatgttatatcccgccgttaaccaccatcaaacaggattttcgcctgctgggg | |
| | caaaccagcgtggaccgcttgctgcaactctctcagggccaggcggtgaagggcaatcagctgt | |
| | tgcccgtctcactggtgaaaagaaaaaccaccctggcgcccaatacgcaaaccgcctctccccg | |
| | cgcgttggccgattcattaatgcagctggcacgacaggtttcccgactggaaagcgggcagtga | |
| | gcgcaacgcaattaatgtgagttagcgcgaattgatctggtttgacagcttatcatcgactgcacgg | |
| | tgcaccaatgcttctggcgtcaggcagccatcggaagctgtggtatggctgtgcaggtcgtaaatc | |
| | actgcataattcgtgtcgctcaaggcgcactcccgttctggataatgttttttgcgccgacatcataa | |
| | cggttctggcaaatattctgaaatgagctgttgacaattaatcatccggctcgtataatgtgtggaatt | |
| | gtgagcggataacaatttcacacaggaaacagac | |
| Laclq- | ctagctacgttgacaccatcgaatggtgcaaaacctttcgcggtatggcatgatagcgcccggaa | 298 |
| trcRBS | gagagtcaattcagggtggtgaatgtgaaaccagtaacgttatacgatgtcgcagagtatgccggt | |
| | gtctcttatcagaccgtttcccgcgtggtgaaccaggccagccacgtttctgcgaaaacgcggga | |
| | aaaagtggaagcggcgatggcggagctgaattacattcccaaccgcgtggcacaacaactggc | |
| | gggcaaacagtcgttgctgattggcgttgccacctccagtctggccctgcacgcgccgtcgcaaa | |
| | ttgtcgcggcgattaaatctcgcgccgatcaactgggtgccagcgtggtggtgtcgatggtagaa | |
| | cgaagcggcgtcgaagcctgtaaagcggcggtgcacaatcttctcgcgcaacgcgtcagtggg | |
| | ctgatcattaactatccgctggatgaccaggatgccattgctgtggaagctgcctgcactaatgttc | |
| | cggcgttatttcttgatgtctctgaccagacacccatcaacagtattattttctcccatgaagacggta | |
| | cgcgactgggcgtggagcatctggtcgcattgggtcaccagcaaatcgcgctgttagcgggccc | |
| | attaagttctgtctcggcgcgtctgcgtctggctggctggcataaatatctcactcgcaatcaaattc | |
| | agccgatagcggaacgggaaggcgactggagtgccatgtccggttttcaacaaaccatgcaaat | |
| | gctgaatgagggcatcgttcccactgcgatgctggttgccaacgatcagatggcgctgggcgca | |
| | atgcgcgccattaccgagtccgggctgcgcgttggtgcggatatctcggtagtgggatacgacga | |
| | taccgaagacagctcatgttatatcccgccgttaaccaccatcaaacaggattttcgcctgctgggg | |
| | caaaccagcgtggaccgcttgctgcaactctctcagggccaggcggtgaagggcaatcagctgt | |
| | tgcccgtctcactggtgaaaagaaaaaccaccctggcgcccaatacgcaaaccgcctctccccg | |
| | cgcgttggccgattcattaatgcagctggcacgacaggtttcccgactggaaagcgggcagtga | |
| | gcgcaacgcaattaatgtgagttagcgcgaattgatctggtttgacagcttatcatcgactgcacgg | |
| | tgcaccaatgcttctggcgtcaggcagccatcggaagctgtggtatggctgtgcaggtcgtaaatc | |
| | actgcataattcgtgtcgctcaaggcgcactcccgttctggataatgttttttgcgccgacatcataa | |
| | cggttctggcaaatattctgaaatgagctgttgacaattaatcatccggctcgtataatgtgtggaatt | |
| | gtgagcggataacaatttcacacaggaaacagagaattcaaaggaggacaac | |
| C. | Ctagcctaaaaacgaccgagcctattgggattaccattgaagccagtgtgagttgcatcacattgg | 299 |
| glutamicum | cttcaaatctgagactttaatttgtggattcacgggggtgtaatgtagttcataattaaccccattcgg | |
| gpd | gggagcagatcgtagtgcgaacgatttcaggttcgttccctgcaaaaactatttagcgcaagtgttg | |
| | gaaatgcccccgtttggggtcaatgtccatttttgaatgtgtctgtatgattttgcatctgctgcgaaat | |
| | ctttgtttccccgctaaagttgaggacaggttgacacggagttgactcgacgaattatccaatgtga | |
| | gtaggtttggtgcgtgagttggaaaaattcgccatactcgcccttgggttctgtcagctcaagaattc | |
| | ttgagtgaccgatgctctgattgacctaactgcttgacacattgcatttcctacaatctttagaggaga | |
| | cacaac | |
| C. | ctagcggggttgctgcactttttaaaaaggcaaaaaatagcgaaaacacaccccaggtttttcccgt | 300 |
| glutamicum | aaccccgctaggctatgcaatttcggtttaacccagtttttcaaagaaggtcactagcttttccgctg | |
| rplM | gtcaccttctttttggtttttcaacgcagagatagtacactttactctttgtgtgtggagtcaaacctccc | |
| | ctttaaggggtgcgcttggacagcaggacaaattcgggtcaccaccggccgccgaatttagcttc | |
| | cttccgaacatattcctggctggcagttctagaccgactaattcaaggagtcattc | |
| C. | ctagctatttcagtgcggggcagtgaaagtaaaaacgcaactttcttacagaacagggttgtctttc | 301 |
| glutamicum | agacgactatgtggttaactacttgggctgctttaacacggcgtgaattaaccatgccagttggtaa | |
| rpsJ | ggcaaacatgacaccttcaattggagtcgaggcgcatgaaaatgcacttcaacttcagggggtat | |
| | ccactgaagccgggtgactggtgaaggcggaaccggagaaggggcatggcaaataaacagcg | |
| | gcagttacgttagggcctagatcacgcattttggtcccttccgatttccctgacttcattgttgggttca | |
| | tcgtggagcgttttatttgtacagcgcccgtgatccaatgtcagaagcatttgacaggtcaggttaaa | |
| | cactggcgttgcgcccgagccccaagcccggacaacgttatagagaaagaatgaagcgaattcc | |
| | caccgcttttccaaaatggaagatgtgggacgagcgaggaagaggataagc | |

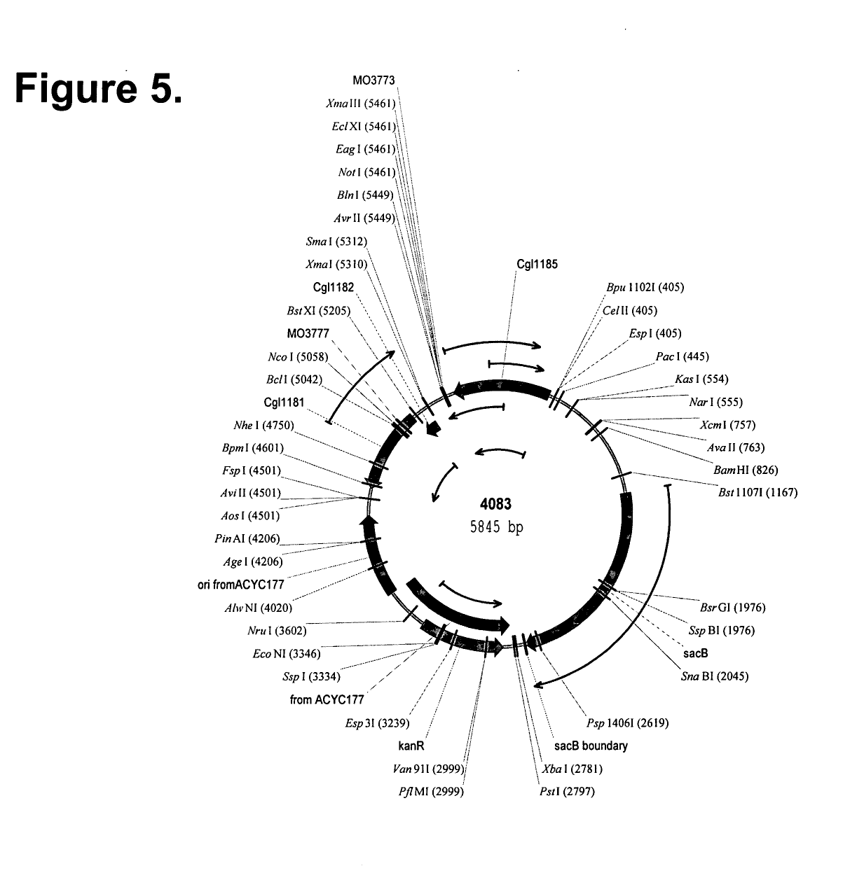

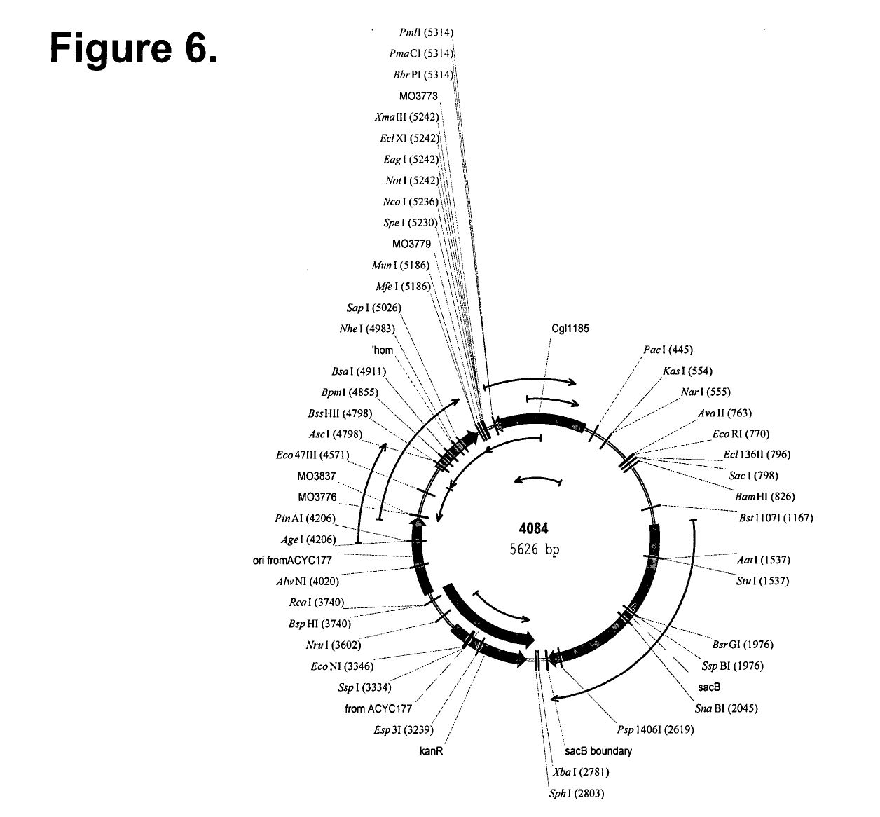

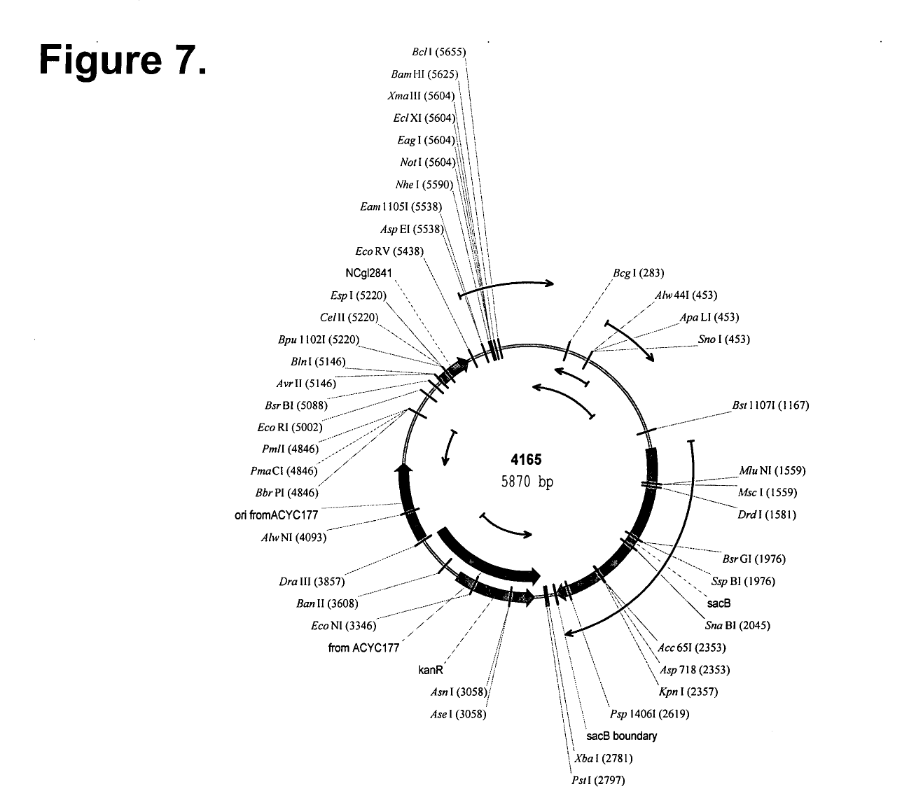

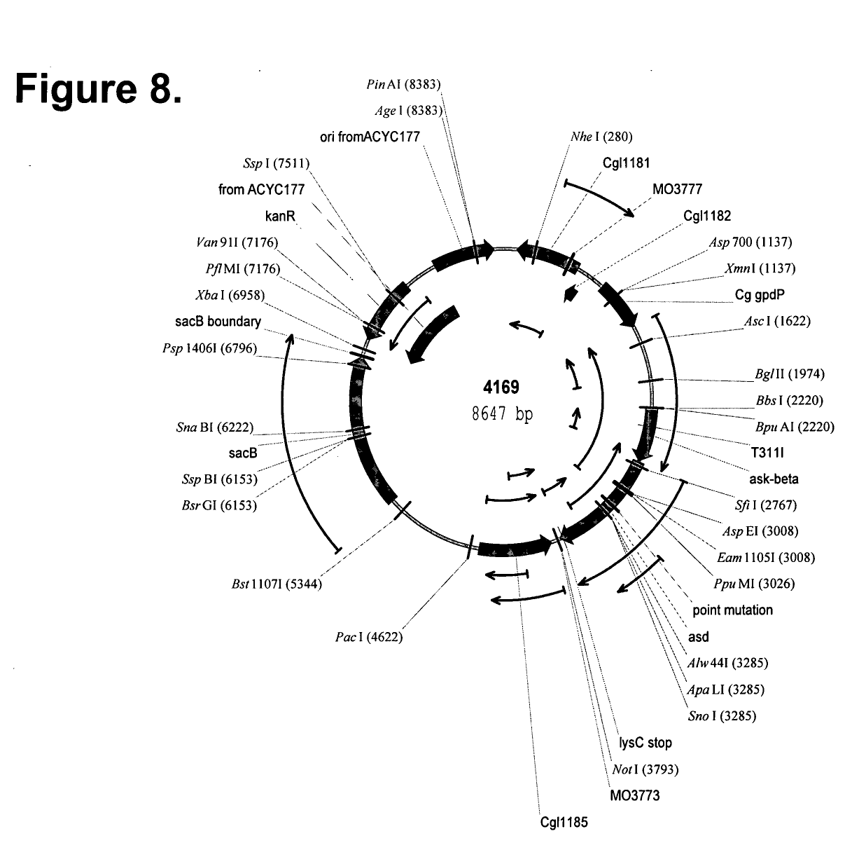

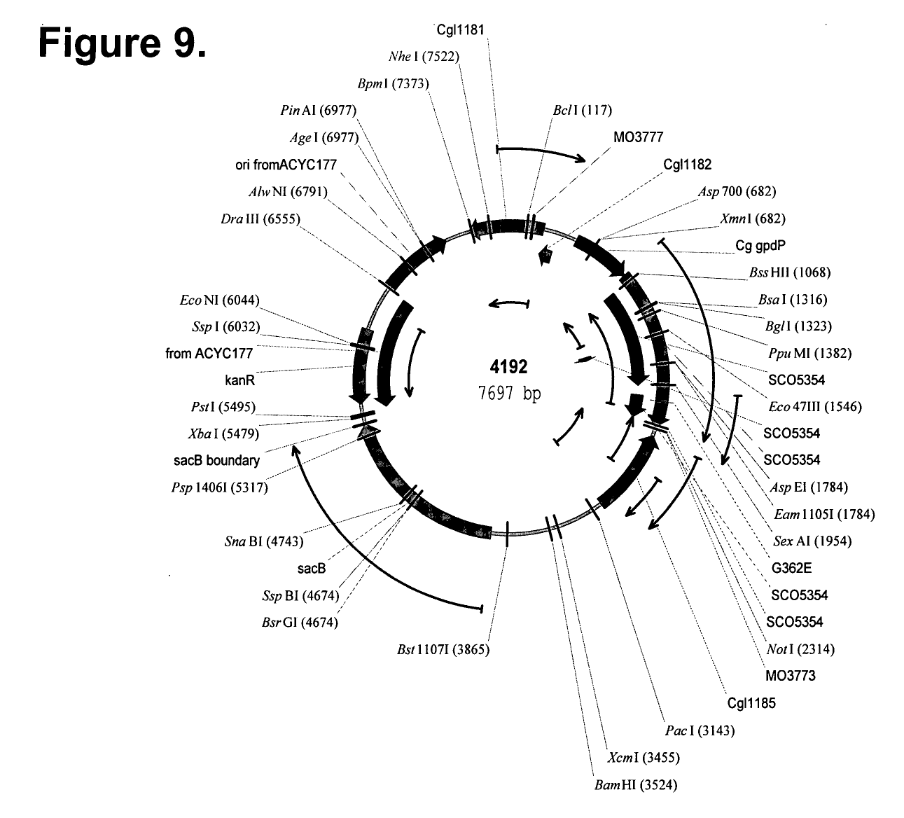

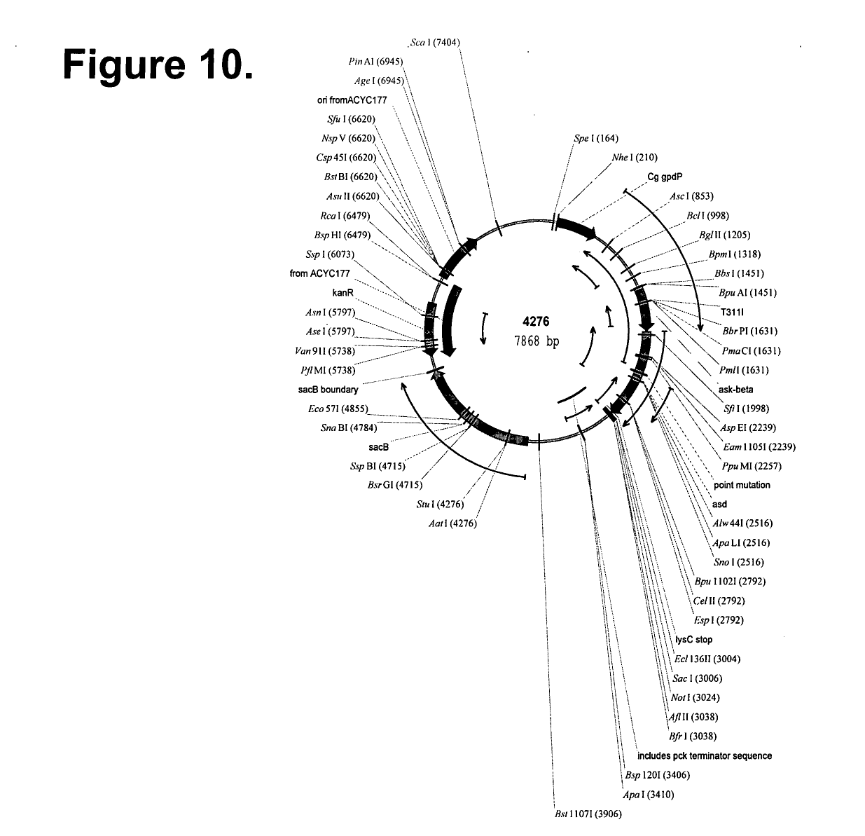

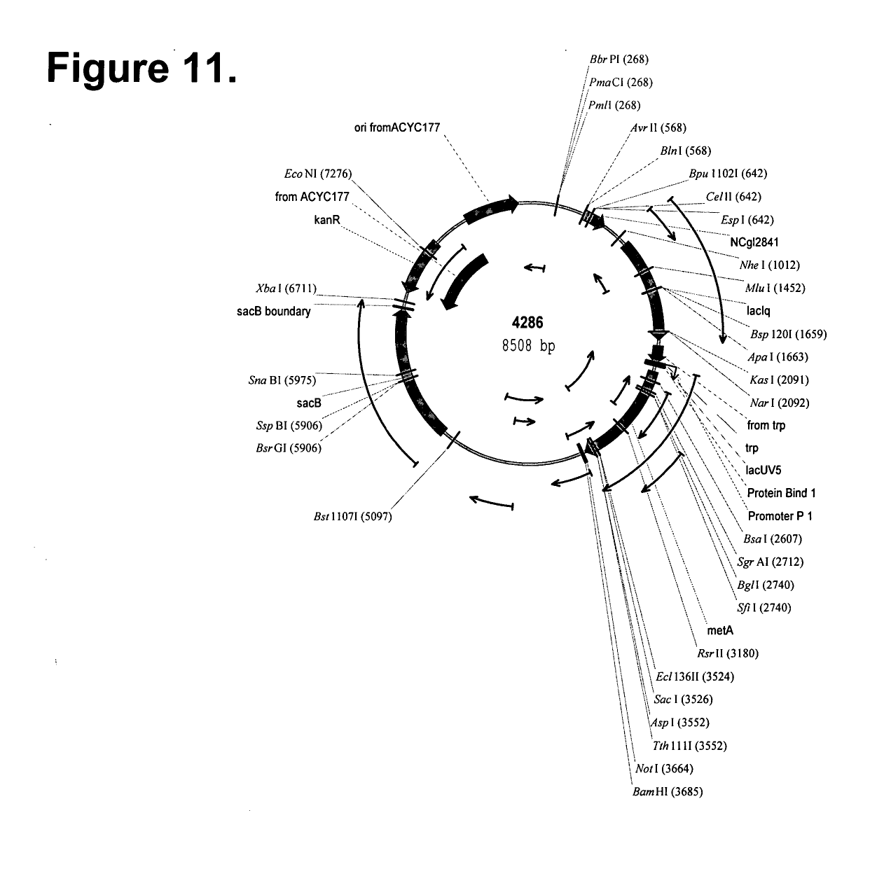

Plasmids were also designed to inactivate native C. glutamicum genes by gene deletion. In some instances, these constructs both delete native genes and insert heterologous genes into the host chromosome at the locus of the deletion event. Table 8 lists the endogenous gene that was deleted and the heterologous genes that were introduced, if any. Deletion plasmids contain nucleotide sequences homologous to regions upstream and downstream of the gene that is the target for the deletion event; in some instances these sequences include small amounts of coding sequence of the gene that is to be inactivated. These flanking sequences are used to facilitate homologous recombination. Single cross-over events target the plasmid into the host chromosome at sites upstream or downstream of the gene to be deleted. Deletion plasmids also contain the sacB gene, encoding the levansucrase gene from Bacillus subtilis. Transformants containing integrated plasmids were streaked to BHI medium lacking kanamycin. After 1 day, colonies were streaked onto BHI medium containing 10% sucrose. This protocol selects for strains in which the sacB gene has been excised, since it polymerizes sucrose to form levan that is toxic to C. glutamicum (see Jager, W., et al. J. Bacteriol. 174:5462-5465, 1992). During growth of transformants upon medium containing sucrose, sacB allows for positive selection for recombination events, resulting in either a clean deletion event or removal of all portions of the integrating plasmid except for the cassette that regulates the inducible expression of a particular gene of interest (see Jager, W., et al. J. Bacteriol. 174:5462-5465, 1992). PCR, together with growth on diagnostic media, was used to verify that expected recombination events have occurred in sucrose-resistant colonies. FIGS. 5-12A display deletion plasmids described herein.

TABLE 8
Plasmids used for deletion of C. glutamicum genes, sometimes
in conjunction with insertion of expression cassettes.

| | Native gene(s) | |
|---|---|---|
| Plasmid | deleted | Element inserted at locus |
| MB4083 | hom-thrB | None |
| MB4084 | thrB | None |
| MB4165 | mcbR | None |
| MB4169 | hom-thrB | gpd-M. smegmatis |
| | | lysC(T311I)-asd |
| MB4192 | hom-thrB | gpd-S. coelicolor |
| | | hom(G362E) |
| MB4276 | pck | gpd-M. smegmatis |
| | | lysC(T311I)-asd |
| MB4286 | mcbR | trcRBS-T. fusca metA |
| MB4287 | mcbR | trcRBS-C. glutamicum metA |
| | | (K233A)-metB |

### EXAMPLE 2

### Isolation of Genes for Enhancing Production of Aspartate-Derived Amino Acids

Wild-type alleles of aspartokinase alpha (lysC-alpha) and beta (lysC-beta) and aspartate semialdehyde dehydrogenase (asd) from Mycobacterium smegmatis (homologs of lysC/asd in Corynebacterium glutamicum); genes encoding aspartokinase-asd (lysC-asd), dapA, and hom from Streptomyces coelicolor; metA and metYA from Thermobifida fusca; and dapA and ppc from Erwinia chrysanthemi are obtained by PCR amplification using genomic DNA isolated from each organism. In addition, in some cases the corresponding wild-type allele for each gene is isolated from C. glutamicum. Amplicons are subsequently cloned into pBluescriptSK II⁻ for sequence verification; in particular instances, site-directed mutagenesis to create the activated alleles is also performed in these vectors. Genomic DNA is isolated from M. smegmatis grown in BHI medium for 72 h at 37° C. using QIAGEN Genomic-tips according to the recommendations of the manufacturer kits (Qiagen, Valencia, Calif.). For the isolation of genomic DNA from S. coelicolor, the Salting Out Procedure (as described in Practical Streptomyces Genetics, pp. 169-170, Kieser, T., et. al., John Innes Foundation, Norwich, England 2000) is used on cells grown in TYE media (ATCC medium 1877 ISP Medium 1) for 7 days at 25° C.

To isolate genomic DNA from T. fusca, cells are grown in TYG media (ATCC medium 741) for 5 days at 50° C. The 100 ml culture is spun down (5000 rpm for 10 min at 4° C.) a washed twice with 40 ml 10 mM Tris, 20 mM EDTA pH 8.0. The cell pellet is brought up in a final volume of 40 ml of 10 mMTris, 20 mM EDTA pH 8.0. This suspension is passed through a Microfluidizer (Microfluidics Corporation, Newton Mass.) for 10 cycles and collected. The apparatus is rinsed with an additional 20 ml of buffer and collected. The final volume of lysed cells is 60 ml. DNA is precipitated from the suspension of lysed cells by isopropanol precipitation, and the pellet is resuspended in 2 ml TE pH 8.0. The sample is extracted with phenol/chloroforn and the DNA precipitated once again with isopropanol. To isolate DNA from E. chrysanthemi, genomic DNA was prepared as described for E. coli (Qiagen genomic protocol) using a Genomic Tip 500/G.

For PCR amplification of the M. smegmatis IysC-asd operon, primers are designed according to sequence upstream of the lysC gene and sequence near the stop of asd. The upstream primer is 5′-CCGTGAGCTGCTCGGATGTGACG-3′ (SEQ ID NO:302), the downstream primer is 5′-TCAGAGGTCGGCGGCCAACAGTTCTGC-3′ (SEQ ID NO:303). The genes are amplified using Pfu Turbo (Stratagene, La Jolla, Calif.) in a reaction mixture containing 10 μl 10× Cloned Pfu buffer, 8 μl dNTP mix (2.5 mM each), 2 μl each primer (20 uM), 1 μl Pfu Turbo, 10 ng genomic DNA and water in a final reaction volume of 100 μl. The reaction conditions are 94° C. for 2 min, followed by 28 cycles of 94° C. for 30 sec, 60° C. for 30sec, 72° C. for 9 min. The reaction is completed with a final extension at 72° C. for 4 min, and the reaction is then cooled to 4° C. The resulting product is purified by the Qiagen gel extraction protocol followed by blunt end ligation into the SmaI site of pBluescript SK II−. Ligations are transformed into E. coli DH5α and selected by blue/white screening. Positive transformants are treated to isolate plasmid DNA by Qiagen methods and sequenced. MB3902 is the resulting plasmid containing the expected insert.

Primer pairs for amplifying S. coelicolor genes are: 5′-ACCGCACTTTCCCGAGTGAC-3′ (SEQ ID NO:304) and 5′-TCATCGTCCGCTCTTCCCCT-3′ (lysC-asd) (SEQ ID NO:305); 5′-ATGGCTCCGACCTCCACTCC-3′ (SEQ ID NO:306) and 5′-CGTGCAGAAGCAGTTGTCGT-3′ (dapA) (SEQ ID NO:307); and 5′-TGAGGTCCGAGGGAGGGAAA-3′ (SEQ ID NO:308) and 5′-TTACTCTCCTTCAACCCGCA-3′ (hom) (SEQ ID NO:309). The primer pair for amplifying the metYA operon from T. fusca is 5′- CATCGACTACGCCCGTGTGA-3′ (SEQ ID NO:310) and 5′-TGGCTGTTCTTCACCGCACC-3′ (SEQ ID NO:311). Primer pairs for amplifying E. chrysanthemi genes are: 5′- TTGACCTGACGCTTATAGCG-3′ (SEQ ID NO:312) and 5′-CCTGTACAAAATGTTGGGAG-3′ (dapA) (SEQ ID NO:313); and 5′-ATGAATGAACAATATTCCGCCA-3′ (SEQ ID NO:314) and 5′-TTAGCCGGTATTGCGCATCC-3′ (ppc) (SEQ ID NO:315).

Amplification of genes was done by similar methods as above or by using the TripleMaster PCR System from Eppendorf (Eppendorf, Hamburg, Germany). Blunt end ligations were performed to clone amplicons into the SmaI site of pBluescript SK II−. The resulting plasmids were MB3947 (S. coelicolor lysC-asd), MB3950 (S. coelicolor dapA), MB4066 (S. coelicolor hom), MB4062 (T. fusca metYA), MB3995 (E. chrysanthemi dapA), and MB4077 (E. chrysanthemippc). These plasmids were used for sequence verification of inserts and subsequent cloning into expression vectors; a subset of these vectors was also subjected to site-directed mutagenesis to generate deregulated alleles of specific genes.

### EXAMPLE 3

### Targeted Substitutions to Enhance the Activity of Genes Involved in the Production of Aspartate-Derived Amino Acids

Site-directed mutagenesis was performed on several of the pBluescript SK II− plasmids containing the heterologous genes described in Example 2. Site-directed mutagenesis was performed using the QuikChange Site-Directed Mutagenesis Kit from Stratagene. For heterologous aspartokinase (lysC/ask) genes, substitution mutations were constructed that correspond to the T311I, S301Y, A279P, and G345D amino acid substitutions in the C. glutamicum protein. These substitutions may decrease feedback inhibition by the combination of lysine and threonine. In all instances, the mutated lysC/ask alleles were expressed in an operon with the heterologous asd gene. Oligonucleotides employed to construct M. smegmatis feedback resistant lysC alleles were: 5′-GGCAAGACCGACATCATATTCACGTGTGCGCGTG-3′ (SEQ ID NO:316) and 5′-CACGCGCACACGTGAATATGATGTCGGTCTTGCC-3′ (T3 11I) (SEQ ID NO:317); 5′-GGTGCTGCAGAACATCTACAAGATCGAGGACGGCAA-3′ (SEQ ID NO:318) and 5′-TTGCCGTCCTCGATCTTGTAGATGTTCTGCAGCACC-3′ (S301Y) (SEQ ID NO:319); 5′-GACGTTCCCGGCTACGCCGCCAAGGTGTTCCGC-3′ (SEQ ID NO:320) and 5′-GCGGAACACCTTGGCGGCGTAGCCGGGAACGTC-3′ (A279P) (SEQ ID NO:321); and 5′-GTACGACGACCACATCGACAAGGTGTCGCTGATCG-3′ (SEQ ID NO:322); and 5′-CGATCAGCGACACCTTGTCGATGTGGTCGTCGTAC-3′ (G345D) (SEQ ID NO:323). Oligonucleotides employed to construct S. coelicolor feedback resistant lysC alleles were: 5′-CGGGCCTGACGGACATCRTCTTCACGCTCCCCAAG-3′ (SEQ ID NO:324) and 5′-CTTGGGGAGCGTGAAGAYGATGTCCGTCAGGCCCG-3′ (S3141/S314V) (SEQ ID NO:325); and 5′-GTCGTGCAGAACGTGTACGCCGCCTCCACGGGC-3′ (SEQ ID NO:326) and 5′-GCCCGTGGAGGCGGCGTACACGTTCTGCACGAC-3′ (S304Y) (SEQ ID NO:327).

Site-directed mutagenesis can be performed to generate deregulated alleles of additional proteins relevant to the production of aspartate-derived amino acids. For example, mutations can be generated that correspond to the V59A, G378E, or carboxy-terminal truncations of the C. glutamicum hom gene. The Transformer Site-Directed Mutagenesis Kit (BD Biosciences Clontech) was used to generate the S. coelicolor hom (G362E) substitution. Oligonucleotides 5′-GTCGACGCGTCTTAAGGCATGCAAGC-3′ (SEQ ID NO:328) and 5′-CGACAAACCGGAAGTGCTCGCCC-3′ (SEQ ID NO:329) were utilized to construct the mutation. Site-directed mutagenesis was also employed to generate specific alleles of the T. fusca and C. glutamicum metA and metY genes (see examples 5 and 6 of the instant specification). Similar strategies can be used to construct deregulated alleles of additional pathway proteins. For example, oligonucleotides 5′-TTCATCGAACAGCGCTCGCACCTGCTGACCGCC-3′ (SEQ ID NO:330) and 5′-GGCGGTCAGCAGGTGCGAGCGCTGTTCGATGAA-3′ (SEQ ID NO:331)can be used to generate a substitution in the S. coelicolor pyc gene that corresponds to the C. glutamicum pyc P458S mutation. Site-directed mutagenesis can also be utilized to introduce substitutions that correspond to deregulated dapA alleles described above.

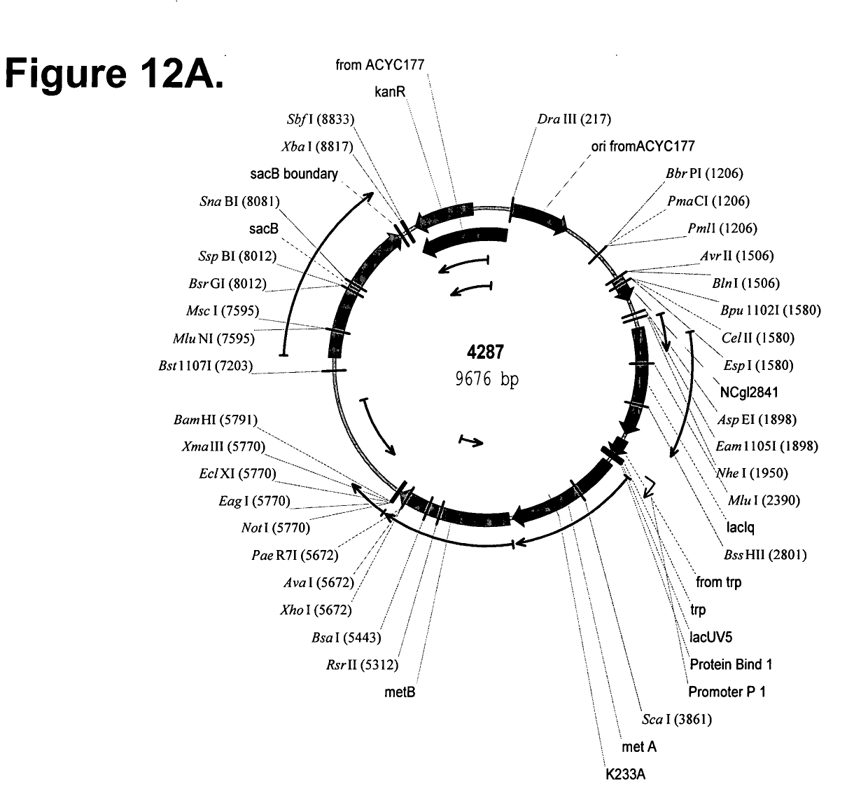

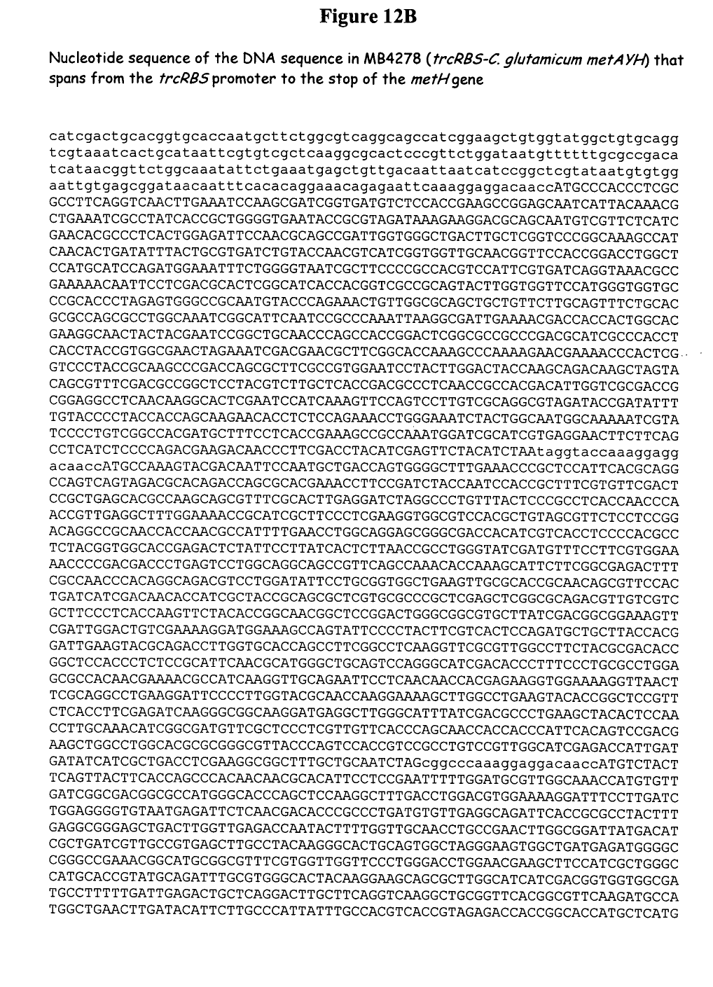

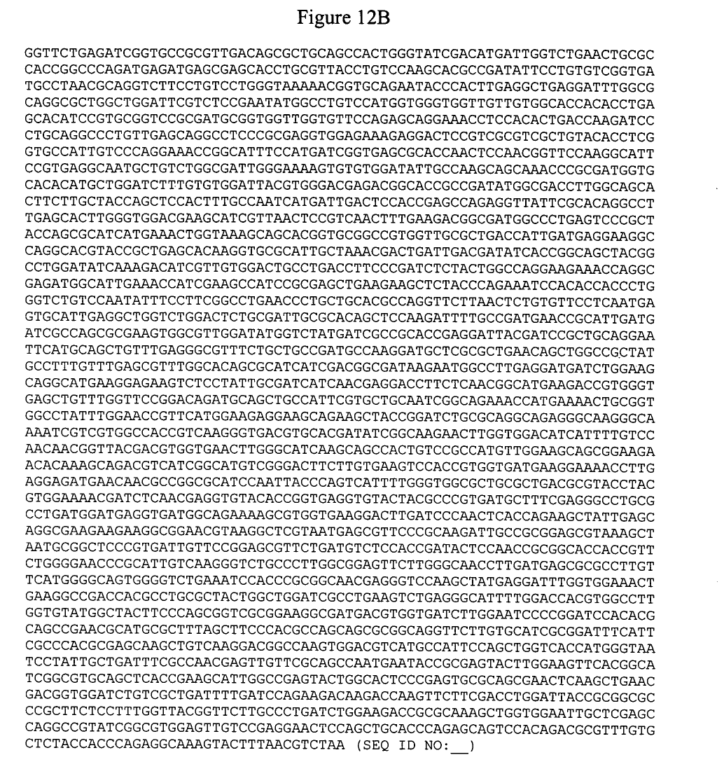

Wild-type and deregulated alleles of heterologous (and C. glutamicum) genes were then cloned into vectors suitable for expression. In general, PCR was employed using oligonucleotides to facilitate cloning of genes as a NcoI-NotI fragment. DNA sequence analysis was performed to verify that mutations were not introduced during rounds of amplification. In some instances, synthetic operons were constructed in order to express two or more genes, heterologous or endogenous, from the same promoter. As an example, plasmid MB4278 was generated to express the C. glutamicum metA, metY, and metH genes from the trcRBS promoter. FIG. 12B displays the DNA sequence in MB4278 that spans from the trcRBS promoter to the stop of the metH gene; the gene order in this construct is metA YH. The open reading frames in FIG. 12B are shown in uppercase. Note that the construct was engineered such that each open reading frame is preceded by an identical stretch of DNA. This conserved sequence serves as a ribosomal-binding sequence that promotes efficient translation of C. glutamicum proteins. Similar intergenic sequences were used to construct additional synthetic operons.

### EXAMPLE 4

### Isolation of Additional Threonine-Insensitive Mutants of Homoserine Dehydrogenase

The hom gene cloned from S. coelicolor in Example 2 is subjected to error prone PCR using the GeneMorph® Random Mutagenesis kit obtained from Stratagene. Under the conditions specified in this kit, oligonucleotide primers 5′-CACACGAAGACACCATGATGCGTACGCGTCCGCT-3′ (contains a BbsI site and cleavage yields a NcoI compatible overhang) (SEQ ID NO:332) and 5′-ATAAGAATGCGGCCGCTTACTCTCCTTCAACCCGCA-3′ (contains a NotI site) (SEQ ID NO:333) are used to amplify the hom gene from plasmid MB4066. The resulting mutant population is digested with BbsI and NotI, ligated into NcoI/NotI digested episomal plasmid containing the trcRBS promoter in the MB4094 plasmid backbone, and transformed into C. glutamicum ATCC 13032. The transformed cells are plated on agar plates containing a defined medium for corynebacteria (see Guillouet, S., et al. Appl. Environ. Microbiol. 65:3100-3107, 1999) containing kanamycin (25 mg/L), 20 mg/L of AHV (alpha-amino, beta-hydroxyvaleric acid; a threonine analog) and 0.01 mM IPTG. After 72 h at 30° C., the resulting transformants are subsequently screened for homoserine excretion by replica plating to a defined medium agar plate supplemented with threonine, which was previously spread with ˜10⁶ cells of indicator C. glutamicum strain MA-331 (hom-thrBA). Putative feedback-resistant mutants are identified by a halo of growth of the indicator strain surrounding the replica-plated transformants. From each of these colonies, the hom gene is PCR amplified using the above primer pair, the amplicon is digested as above, and ligated into the episomal plasmid described above. Each of these putative hom mutants is subsequently re-transformed into C. glutamicum ATCC 13032 and plated on minimal medium agar plates containing 25 mg/L kanamycin and 0.01 mM IPTG. One colony from each transformation is replica plated to defined medium for corynebacteria containing 10, 20, 50, and 100 mg/L of AHV, and sorted based on the highest level of resistance to the threonine analog. Representatives from each group are grown in minimal medium to an OD of 2.0, the cells harvested by centrifugation, and homoserine dehydrogenase activity assayed in the presence and absence of 20 mM threonine as referenced in Chassagnole, C., et al., Biochem. J. 356:415-423, 2001. The hom gene is PCR amplified from those cultures showing feedback-resistance and sequenced. The resulting plasmids are used to generate expression plasmids to enhance amino acid production.

### EXAMPLE 5

### Isolation of Feedback-Resistant Mutants of Homoserine O-Acetyltransferase (metA) and O-Acetylhomoserine Sulfhydrylase (metY)

The heterologous metA gene cloned from T. fusca is subjected to error prone PCR using the GeneMorph® Random Mutagenesis kit obtained from Stratagene. Under the conditions specified in this kit, oligonucleotide primers 5′-CACACACCTGCCACACATGAGTCACGACACCACCCCTCC-3′ (contains a BspMI site and cleavage yields a NcoI compatible overhang) (SEQ ID NO:334) and 5′-ATAAGAATGCGGCCGCTTACTGCGCCAGCAGTTCTT-3′ (contains a NotI site) (SEQ ID NO:335) are used to amplify the metA gene from plasmid MB4062. The resulting mutant amplicon is digested and ligated into the NcoIlNotI digested episomal plasmid described in Example 4, and then transformed into C. glutamicum strain MA-428. MA-428 is a derivative of ATCC 13032 that has been transformed with integrating plasmid MB4192. After selection for recombination events, the resulting strain MA-428 is deleted for hom-thrB in a manner that results in insertion of a deregulated S. coelicolor hom gene. The transformed MA-428 cells described are plated on minimal medium agar plates containing kanamycin (25 mg/L), 0.01 mM IPTG, and 100 μg/ml or 500 μg/ml of trifluoromethionine (TFM; a methionine analog). After 72 h at 30° C., the resulting transformants are subsequently screened for O-acetylhomoserine excretion by replica plating to a minimal agar plate which was previously spread with ˜10⁶ cells of an indicator strain, S. cerevisiae B-7588 (MATa ura3-5Z ura3-58, leu2-3, leu2-112, trp1-289, met2, HIS3+), obtained from ATCC (#204524). Putative feedback-resistant mutants are identified by the excretion of O-acetylhomoserine (OAH), which supports a halo of indicator strain growth surrounding the replica-plated transformants.

From each of these cross-feeding colonies, the metA gene is PCR amplified using the above primer pair, digested with BspMI and NotI, and ligated into the NotI/NcoI digested episomal plasmid described in example 4. Each of these putative metA mutant alleles is subsequently re-transformed into C. glutamicum ATCC 13032 and plated on minimal medium agar plates containing 25 mg/L kanamycin. One colony from each transformation is replica plated to minimal medium containing 100, 200, 500, and 1000 μg/ml of TFM plus 0.01 mM IPTG, and sorted based on the highest level of resistance to the methionine analog. Representatives from each group are grown in minimal medium to an OD of 2.0, the cells harvested by centrifugation, and homoserine O-acetyltransferase activity is determined by the methods described by Kredich and Tomkins (J. Biol. Chem. 241:4955-4965,1966) in the presence and absence of 20 mM methionine or S-AM. The metA gene is PCR amplified from those cultures showing feedback-resistance and sequenced. The resulting plasmids are used to generate expression plasmids to enhance amino acid production. In a similar manner, the metY gene from T. fusca is subjected to mutagenic PCR. Oligonucleotide primers 5′-CACAGGTCTCCCATGGCACTGCGTCCTGACAGGAG-3′ (contains a BsaI site and cleavage yields a NcoI compatible overhang) (SEQ ID NO:336) and 5′-ATAAGAATGCGGCCGCTCACTGGTATGCCTTGGCTG-3′ (contains a NotI site) (SEQ ID NO:337) are used for cloning into the episomal plasmid, as described above, and for carrying out the mutagenesis reaction per the specifications of the GeneMorph® Random Mutagenesis kit obtained from Stratagene. The major difference is that the mutated metYpopulation is transformed into a C. glutamicum strain that already produces high levels of O-acetylhomoserine. This strain, MICmet2, is constructed by transforming MA-428 with a modified version of plasmid MB4286 that contains a deregulated T. fusca metA allele described above under the control of the trcRBS promoter. After transformation the sacB selection system enables the deletion of the endogenous mcbR locus and replacement with the deregulated heterologous metA allele.

The T. fusca metY variant transformed MICmet2 strain is spread onto minimal agar plates containing 25 mg/L of kanamycin, 0.25mM IPTG, and an inhibiting concentration of toxic methionine analog(s) (e.g., ethionine, selenomethionine, TFM); the transfornants can be grown on these 3 different methionine analogs either individually or in double or triple combination). The metY gene is amplified from those colonies growing on the selection plates, the amplicons are digested and ligated into the episomal plasmid described in example 4, and the resulting plasmids are transformed into MICmet2. The transformants are grown on minimal medium agar plates containing 25 mg/L of kanamycin. The resulting colonies are replica-plated to agar plates containing a 10-fold range of the toxic methionine analogs ethionine, TFM, and selenomethionine (plus 0.01 mM IPTG), and sorted on the basis of analog sensitivity. Representatives from each group are grown in minimal medium to an OD of 2.0, the cells are harvested by centrifugation, and O-acetylhomoserine sulfhydrylase enzyme activity is determined by a modified version of the methods of Kredich and Tomkins (J. Biol. Chem. 241:4955-4965,1966) (see example 9) in the presence and absence of 20 mM methionine. The metY gene is PCR amplified from those cultures showing feedback-resistance and sequenced. The resulting plasmids are used to generate expression plasmids to enhance amino acid production. An expression plasmid containing the feedback resistant metY and metA variants from T. fusca is constructed as follows. The T. fusca metYA operon is amplified using oligonucleotides 5′-CACACACATGTCACTGCGTCCTGACAGGAGC-3′ (contains a Pcil site and cleavage yields a NcoI compatible overhang (also changes second codon from Ala>Ser)) (SEQ ID NO:338) and 5′-ATAAGAATGCGGCCGCTTACTGCGCCAGCAGTTCTT -3′ (contains a NotI site) (SEQ ID NO:339). The amplicon is digested with PciI and NotI, and the fragment is ligated into the above episomal plasmid that has been treated sequentially treated with NotI, HaeIII methylase, and NcoI. Site directed mutagenesis, performed using the QuikChange Site-Directed Mutagenesis Kit from Stratagene, is used to incorporate the described substitution mutations in T. fusca metA and metY into a single plasmid that expresses the deregulated alleles as an operon. The resulting plasmid is used to enhance amino acid production.

Minimal medium: 10 g glucose, 1 g NH₄H₂PO₄, 0.2 g KCl, 0.2 g MgSO₄-7H₂O, 30 and 1 ml TE per liter of deionized water (pH 7.2). Trace elements solution (TE) comprises: 88 mg Na₂B₄O₇-10H₂O, 37 mg (NH₄)₆Mo₇O₂₇-4H₂O, 8.8 mg ZnSO₄-7H₂O, 270 mg CuSO₄-5H₂O, 7.2 mg MnCl₂-4H₂O, and 970 mg FeCl₃-6H₂O per liter of deionized water. (When needed to support auxotrophic requirements, amino acids and purines are supplemented to 30 mg/L final concentration.)

### EXAMPLE 6

### Identification of S-AM-Binding Residues in Bacterial Amino Acid Sequences

Many enzymes that regulate amino acid production are subject to allosteric feedback inhibition by S-AM. We hypothesized that variants of these enzymes with resistance to S-AM regulation (e.g., via resistance to S-AM binding or to S-AM-induced allosteric effects) would be resistant to feedback inhibition. S-AM binding motifs have been identified in bacterial DNA methyltransferases (Roth et al., J. Biol. Chem., 273:17333-17342, 1998). Roth et al. identified a highly conserved amino acid motif in EcoRV α-adenine-N⁶-DNA methyltransferase which appeared to be critical for S-AM binding by the enzyme. We searched for related motifs in the amino acid sequences of the following proteins of C. glutamicum: MetA, MetY, McbR, LysC, MetB, MetC, MetE, MetH, and MetK. Putative S-AM binding motifs were identified in MetA, MetY, McbR, LysC, MetB, MetC, MetH, and MetK. We also identified additional residues in metY that are analogous to a S-AM binding motif in a yeast protein. (Pintard et al., Mol. Cell Biol., 20(4):1370-1381, 2000).

Residues of each protein that may be involved in S-AM binding are listed in Table 9.

TABLE 9
Putative residues involved in S-AM
binding in C. glutamicum proteins

| | Putative residue involved |
|---|---|
| Protein | in S-AM binding |
| MetA | G231 |
| | K233 |
| | F251 |
| | V253 |
| | D269 |
| MetY | G227 |
| | L229 |
| | D231 |
| | G232 |
| | G233 |
| | F235 |
| | D236 |
| | V239 |
| | F368 |
| | D370 |
| | D383 |
| | G346 |
| | K348 |
| McbR | G92 |
| | K94 |
| | F116 |
| | G118 |
| | D134 |
| LysC | G208 |
| | K210 |
| | F223 |
| | V225 |
| | D236 |
| MetB | G72 |
| | K74 |
| | F90 |
| | I92 |
| | D105 |
| MetC | G296 |
| | K298 |
| | F312 |
| | G314 |
| | D335 |
| MetH | G708 |
| | K710 |
| | F725 |
| | L727 |
| MetK | G263 |
| | K265 |
| | F282 |
| | G284 |
| | D291 |

Alignment of MetA and MetY sequences from other species was used to identify additional putative S-AM-binding residues. These residues are listed in Table 10.

TABLE 10
Putative S-AM binding amino acids in
bacterial MetA and MetY proteins

| | | Putative residue | |
|---|---|---|---|
| | | involved in S-AM | Homologous Residue |
| Protein | Organism | binding | in C. glutamicum |
| MetY | T. fusca | G240 | G227 |
| | | D244 | D231 |
| | | F379 | F368 |
| | | D394 | D383 |
| MetY | M. tuberculosis | G231 | G227 |
| | | D235 | D231 |
| | | F367 | F368 |
| | | D382 | D383 |
| MetA | T. fusca | G81 | analogous residue |
| | | | absent in |
| | | | C. glutamicum |
| | | D287 | D269 |
| | | F269 | F251 |
| MetA | E. coli | E252 | D269 |
| MetA | M. leprae | G73 | analogous residue |
| | | | absent in |
| | | | C. glutamicum |
| | | D278 | D269 |
| | | Y260 | D269 |
| MetA | M. tuberculosis | G73 | analogous residue |
| | | | absent in |
| | | | C. glutamicum |
| | | Y260 | F251 |
| | | D278 | D269 |

MetA and MetY genes were cloned from C. glutamicum and T. fusca as described in Example 2. Table 11 lists the plasmids and strains used for the expression of wild-type and mutated alleles of MetA and MetY genes. Tables 12 and 13 list the plasmids used for expression and the oligonucleotides employed for site-directed mutagenesis to generate MetA and MetY variants.

### EXAMPLE 7

### Preparation of Protein Extracts for MetA and MetY Assays

A single C. glutamicum colony was inoculated into seed culture media (see example 10 below) and grown for 24 hour with agitation at 33 ° C. The seed culture was diluted 1:20 in production soy media (40 mL) (example 10) and grown 8 hours. Following harvest by centrifugation, the pellet was washed lx in 1 volume of water. The pellet was resuspended in 250 μl lysis buffer (1 ml HEPES buffer, pH 7.5, 0.5 ml 1M KOH, 10 μl 0.5M EDTA, water to 5ml), 30 μl protease inhibitor cocktail, and 1 volume of 0.1 mm acid washed glass beads. The mixture was alternately vortexed and held on ice for 15 seconds each for 8 reptitions. After centrifugation for 5′ at 4,000 rpm, the supernatant was removed and re-spun for 20′ at 10,000 rpm. The Bradford assay was used to determine protein concentration in the cleared supernatant.

### EXAMPLE 8

### Quantifying MetA Activity in C. glutamicum Strains Containing Episomal Plasmids

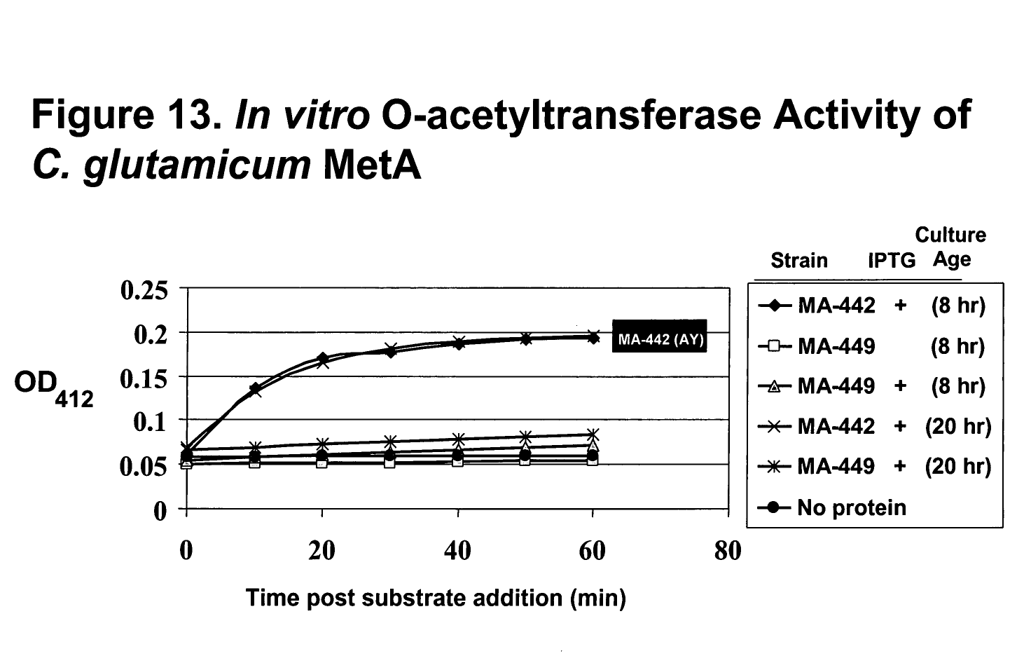

MetA activity in C. glutamicum expressing endogenous and episomal metA genes was determined. MetA activity was assayed in crude protein extracts using a protocol described by Kredich and Tomkins (J. Biol. Chem.241(21):4955-4965, 1966). Preparation of protein extracts is described in the Example 7. Briefly, 1 μg of protein extract was added to a microtiter plate. Reaction mix (250 μl; 100 mM tris-HCl pH 7.5, 2mM 5,5′-Dithiobis(2-nitrobenzoic acid) (DTN), 2 mM sodium EDTA, 2 mM acetyl CoA, 2 mM homoserine) was added to each well of the microtiter plate. In the course of the reactions, MetA activity liberates CoA from acetyl-CoA. A disulfide interchange occurs between the CoA and DTN to produce thionitrobenzoic acid. The production of thionitrobenzoic acid is followed spectrophotometrically. Absorbance at 412 nm was measured every 5 minutes over a period of 30 minutes. A well without protein extract was included as a control. Inhibition of MetA activity was determined by addition of S-adenosyl methionine (S-AM; 0.02 mM, 0.2 mM, 2 mM) and methionine (.5 mM, 5 mM, 50 mM). Inhibitors were added directly to the reaction mix before it was added to the protein extract. In vitro O-acetyltransferase activity was measured in crude protein extracts derived from C. glutamicum strains MA-442 and MA-449 which contain both endogenous and episomal C. glutamicum MetA and MetY genes. Episomal metA and metY genes were expressed as a synthetic operon; the nucleic acid sequence of the metAY operon is as shown in the metAYH operon of FIG. 12B, only lacking metH sequence. The trcRBS promoter was employed in these episomal plasmids. MA-442 expresses the episomal genes in the order metA-metY. MA-449 expresses the episomal genes in the order metY-metA. Experiments were performed in the presence and absence of IPTG that induces expression of the plasmid borne MetA and MetY genes. FIG. 13 shows a time course of MetA activity. MetA activity was observed only when the genes were in the MetA-MetY (MA-442) configuration in samples from 8 hour and 20 hour cultures. In contrast, MetA activity in extracts from strain MA-449 (MetY-MetA) was not significantly elevated relative to a control sample lacking protein at both 8 hour and 20 hour time points, with and without induction. This data is consistent with Northern blot analysis that showed low expression of metA when the two genes were in the metY-metA orientation.

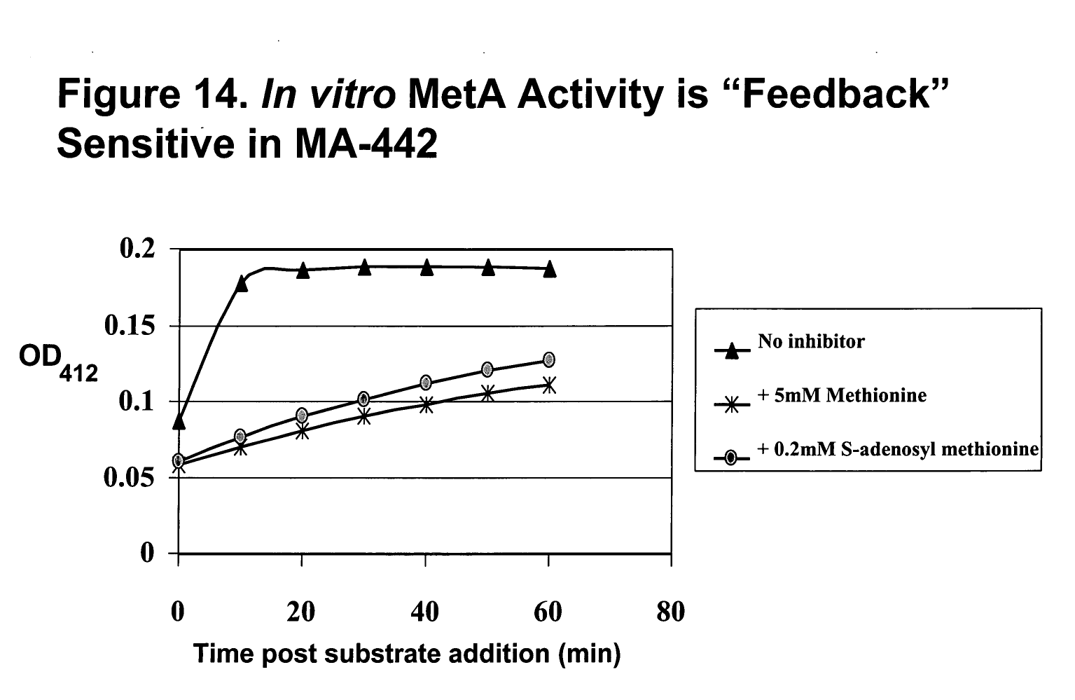

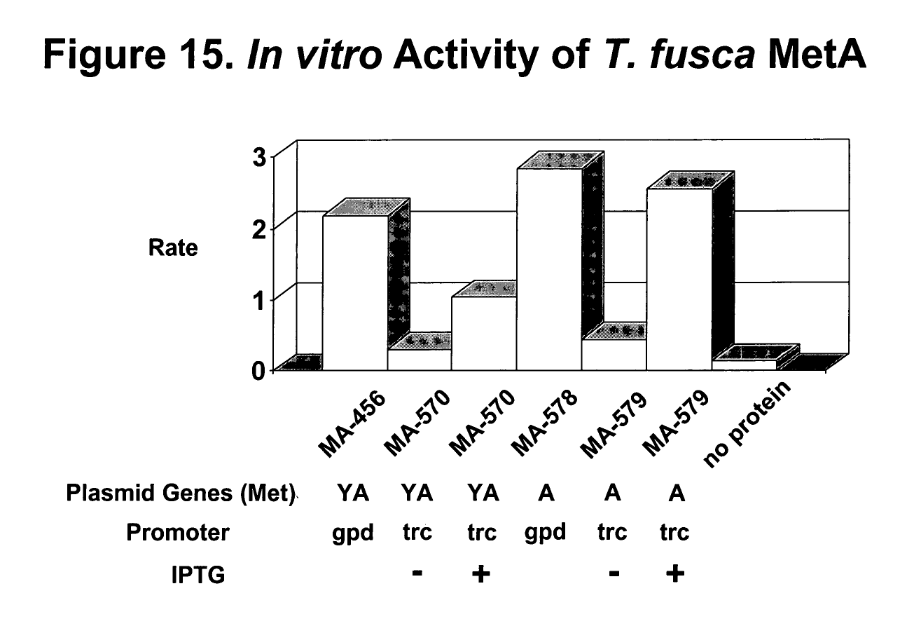

Next, sensitivity of extracts from strain MA-442 to feedback inhibition was tested. MA-442 extracts were assayed in the presence of 5 mM methionine, 0.2 mM S-AM, or in the absence of additional methionine or S-AM, and MetA activity was assayed as described above. As shown in FIG. 14, MetA activity was reduced in the presence of 5 mM methionine and 0.2 mM S-AM. Thus, reducing allosteric repression of MetA may enhance MetA activity, allowing production of higher levels of methionine. It is possible that allosteric repression would also be observed at much lower levels of methionine or S-AM. Regardless, the levels tested are physiologically relevant levels in strains engineered for the production of amino acids such as methionine. C. glutamicum strains expressing episomal T. fusca MetA (strains MA-578 and MA-579), or both episomal T. fusca MetA and MetY (strains MA-456 and MA-570) were constructed and extracts were prepared from these strains and assayed for MetA activity. The regulatory elements associated with each episomal gene are listed in Table 12. The rate of MetA activity in extracts of each strain was determined by calculating the change in OD₄₁₂ divided by time per ng of protein. The results of these assays are depicted in FIG. 15, which shows that strain MA-578 exhibited a rate of approximately 2.75 units (change in OD₄₁₂ /time/ng protein) under inducing conditions, whereas the rate under non-inducing conditions was approximately 1. Strain MA-579 exhibited a rate of approximately 2.5 under inducing conditions and a rate of approximately 0.4 under non-inducing conditions. Strain MA-456, which expresses metA and metYunder the control of a constitutive promoter, exhibited a rate of approximately 2.2. Strain MA-570 exhibited a rate of approximately 1 under inducing conditions and a rate of 0.3 under non-inducing conditions. The negative control sample (no protein) exhibited a rate of approximately 0.1. These data show that episomal expression of T. fusca metA in C. glutamicum increases the rate of MetA activity. The increase was similar to the increase observed with episomal expression of C. glutamicum MetA in C. glutamicum.

### EXAMPLE 9

### Quantifying MetY Activity in C. glutamicum Strains Containing Episomal Plasmids

The in vitro activity of episomal T. fusca MetY was determined in several C. glutamicum strains. MetY activity was assayed in C. glutamicum crude protein extracts using a modified protocol of Kredich and Tomkins (J. Biol. Chem., 241(21):4955-4965, 1966). Crude protein extracts were prepared as described. Briefly, 900 μl of reaction mix (50 mM Tris pH 7.5, 1 mM EDTA, 1 mM sodium sulfide nonahydrate (Na₂S), 0.2mM pyridoxal-5-phosphoric acid (PLP) was mixed with 45 μg of protein extract. At time zero, O-acetyl homoserine (OAH; Toronto Research Chemicals Inc) was added to a final concentration of 0.625 mM. 200 μl of the reaction was removed immediately for the zero time point. The remainder of the reaction was incubated at 30° C. Three 200 μl samples were removed at 10 minute intervals. Immediately after removal from 30° C., the reactions were stopped by the addition of 125 μl 1 mM nitrous acid which nitrosates the thiol groups of homocysteine to form S-nitrosothiol. Five minutes later, 30 μl of 0.5% ammonium sulfamate (removes excess nitrous acid) was added and the sample vortexed. Two minutes later, 400 μl of detection solution (1 part 1% HgCl2 in 0.4N HCl, 4 parts 3.44% % sulfanilamide in 0.4N HCl, 2 parts 0.1% 1-naphthylethylenediamine dihydrochloride in 0.4N HCl) was added and the solution vortexed. In the presence of mercuric ion the S-nitrosothiol rapidly decomposes to give nitrous acid, diazotizing the sulfanilamide, which then couples with the naphthylethylenediamine to give a stable azo dye as a chromaphore. After 5 minutes, the solution was transferred to a microtiter dish and the absorbance at 540 nm was measured. A reaction without protein extract was included as a control.

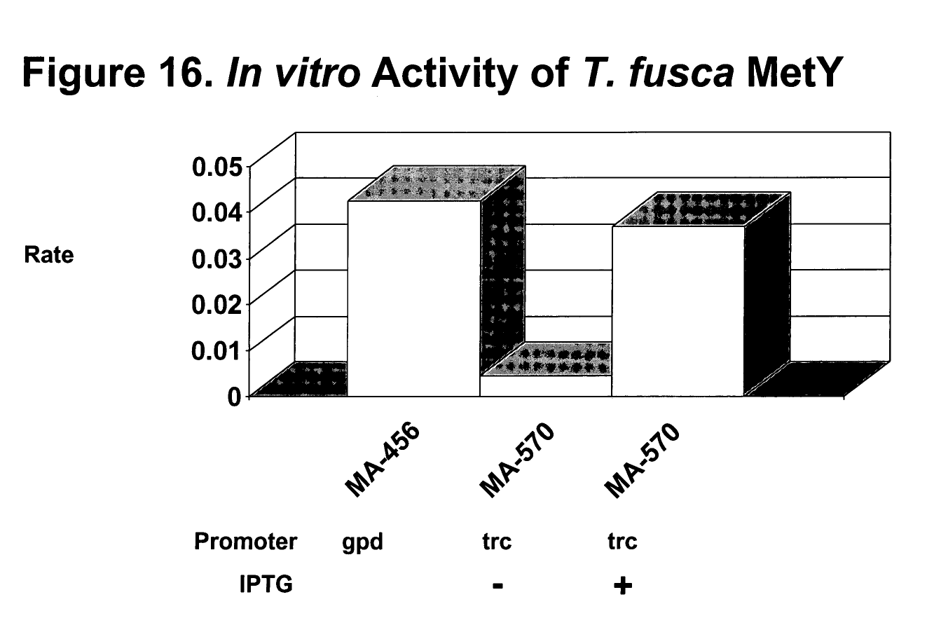

The results of the assays are depicted in FIG. 16. Strain MA-456, which expresses episomal wild type T. fusca metA and metY alleles under the control of a constitutive promoter, exhibited a rate of 0.04. Strain MA-570, which expresses episomal wild type T. fusca metA and metY alleles under the control of an inducible promoter, exhibited a rate of approximately 0.038 under inducing conditions, and a rate of less than 0.01 under non-inducing conditions. Thus, expression of heterologous MetY results in enzyme activity that is significantly elevated over that of the endogenous MetY.

TABLE 11
C. glutamicum strains used to determine activity of MetA and MetY proteins,
and impact of overexpression on production of aspartate-derived amino acids.

| | | | relevant | | |
|---|---|---|---|---|---|
| | relevant | | plasmid | episomal | episomal |
| Strain | strain | episomal | regulatory | metY | metA |
| Name | genotype | plasmid | sequence | species | species |
| MA-2 | n/a | n/a | n/a | n/a | n/a |
| (ATCC | | | | | |
| 13032) | | | | | |
| MA-422 | ethionine resistant | n/a | n/a | n/a | n/a |
| | variant of MA-2 | | | | |
| MA-428 | MA-2 derivative | n/a | n/a | n/a | n/a |
| | with Δhom- ΔthrB:: C | | | | |
| | glutamicum gpd promoter - | | | | |
| | S. coelicolor hom | | | | |
| | (G362E)^{a} | | | | |
| MA-442 | MA-428 derivative | MB-4135^{b} | lacIQ-TrcRBS | Cg wild-type | Cg wild-type |
| MA-449 | MA-428 derivative | MB-4138 | lacIQ-TrcRBS | Cg wild-type | Cg wild-type |
| MA-456 | MA-428 derivative | MB-4168 | gpd | Tf wild-type | Tf wild-type |
| MA-570 | MA-428 derivative | MB-4199 | lacIQ-TrcRBS | Tf wild-type | Tf wild-type |
| MA-578 | MA-428 derivative | MB-4205 | gpd | none | Tf wild-type |
| MA-579 | MA-428 derivative | MB-4207 | lacIQ-TrcRBS | none | Tf wild-type |
| MA-622 | mcbRΔ derivative of | n/a | n/a | n/a | n/a |
| | MA-422 | | | | |
| MA-641 | MA-622 derivative | MB-4136 | gpd | Cg wild-type | Cg wild-type |
| MA-699 | MA-622 derivative | n/a | n/a | n/a | n/a |
| MA-721 | MA-622 derivative | MB-4236^{b} | lacIQ-TrcRBS | Cg wild-type | Cg K233A |
| MA-725 | MA-622 derivative | MB-4238^{b} | lacIQ-TrcRBS | Cg D231A | Cg wild-type |
| MA-727 | MA-622 derivative | MB-4239^{b} | lacIQ-TrcRBS | Cg G232A | Cg wild-type |

abbreviations - Cg (Coryneform glutamicum), Tf (Thermobifida fusca), lacIQ-TrcRBS (see above) (lacIQ-Trc regulatory sequence from pTrc99A (Amann et al., Gene (1988) 69:301-315)); gpd (C. glutamicum gpd promoter)
^{a}the endogenous hom(thrA)-thrB locus was replaced with the S. coelicolor hom (G362E) sequence under the C. glutamicum gpd (glyceraldehyde-3-phosphate dehydrogenase) promoter
^{b}in this plasmid the gene order is MetA-MetY. Unless otherwise indicated, in other plasmids the gene order is MetY-MetA

TABLE 12
Plasmids and oligos used for site directed mutagenesis
to generate MetA and MetY variants.

| Plasmid | oligo 1 | oligo 2 | Gene | wt/variant | Organism |
|---|---|---|---|---|---|
| MB4238 | MO4057 | MO4058 | metY | D231A | C. glutamicum |
| n/a | MO4045 | MO4046 | metY | D244A | T. fusca |
| n/a | MO4041 | MO4042 | metA | D287A | T. fusca |
| n/a | MO4049 | MO4050 | metY | D394A | T. fusca |
| n/a | MO4039 | MO4040 | metA | F269A | T. fusca |
| n/a | MO4047 | MO4048 | metY | F379A | T. fusca |
| MB4239 | MO4059 | MO4060 | metY | G232A | C. glutamicum |
| n/a | MO4043 | MO4044 | metY | G240A | T. fusca |
| n/a | MO4037 | MO4038 | metA | G81A | T. fusca |
| MB4236 | MO4051 | MO4052 | metA | K233A | C. glutamicum |
| MB4135 | n/a | n/a | metA | wt | C. glutamicum |
| MB4135 | n/a | n/a | metY | wt | C. glutamicum |
| MB4210 | n/a | n/a | metY | wt | T. fusca |
| MB4210 | n/a | n/a | metA | wt | T. fusca |

TABLE 13

Sequences of oligos used for site-directed mutagenesis to generate
MetA and MetY variants.

| | | |
|---|---|---|
| Oligo name | Oligo Sequence | SEQ ID NO: |

| | | |
|---|---|---|
| MO4037 | 5′ GTAGGCCCGGAAGGCCCCGCGCACCCCAGCCCAGGCTGG 3′ | 340 |
| MO4038 | 5′ CCAGCCTGGGCTGGGGTGCGCGGGGCCTTCCGGGCGTAC 3′ | 341 |
| MO4039 | 5′ CCGATGGCCGGGGGCGGGGCCGCTGTCGAGTCGTACCTG 3′ | 342 |
| MO4040 | 5′ CAGGTACGACTCGACAGCGGCCCGGCCCCCGGCCATCGG 3′ | 343 |
| MO4041 | 5′ AAACTCGCCCGCCGGTTCGCCGCGGGCAGCTACGTCGTG 3′ | 344 |
| MO4042 | 5′ GACGACGTAGCTGCCCGCGGCGAACCGGCGGGCGAGTTT 3′ | 345 |
| MO4043 | 5′ CACGGCACCACGATCGCGGCCATCGTGGTGGACGCCGGC 3′ | 346 |
| MO4044 | 5′ GCCGGCGTCCACCACGATGGCCGCGATCGTGGTGCCGTG 3′ | 347 |
| MO4045 | 5′ ATCGCGGGCATCGTGGTGGCCGCCGGCACCTTCGACTTC 3′ | 348 |
| MO4046 | 5′ GAAGTCGAAGGTGCCGGCGGCCACCACGATGCCCGCGAT 3′ | 349 |
| MO4047 | 5′ ATCGAGGCCGGACGCGCCGCCGTGGACGGCACCGAACTG 3′ | 350 |
| MO4048 | 5′ CAGTTCGGTGCCGTCCACGGCGGCGCGTCCGGCGTCGAT 3′ | 351 |
| MO4049 | 5′ CAGCTCGTCAACATCGGTGCCGTGCGCAGCCTCATCGTC 3′ | 352 |
| MO4050 | 5′ GACGATGAGGCTGCGCACGGCACCGATGTTGACGAGCTG 3′ | 353 |
| MO4051 | 5′ GACGAACGCTTCGGCACCGCAGCGCAAAAGAACGAAAAC 3′ | 354 |
| MO4052 | 5′ GTTTTCGTTCTTTTGGGCTGCGGTGCCGAAGCGTTCGTC 3′ | 355 |
| MO4057 | 5′ CTGGGCGGCGTGCTTATCGCCGGCGGAAAGTTCGATTGG 3′ | 356 |
| MO4058 | 5′ CCAATCGAACTTTCCGCCGGCGATAAGCACGCCGCCCAG 3′ | 357 |
| MO4059 | 5′ GGCGGCGTGCTTATCGACGCCGGAAAGTTCGATTGGACT 3′ | 358 |
| MO4060 | 5′ AGTCCAATCGAACTTTCCGGCGTCGATAAGCACGCCGCC 3′ | 359 |

### EXAMPLE 10

### Methods for Producing and Detecting Aspartate-Derived Amino Acids

For shake flask production of aspartate-derived amino acids, each strain was inoculated from an agar plate into 10 ml of Seed Culture Medium in a 125 ml Erlenmeyer flask. The seed culture was incubated at 250 rpm on a shaker for 16 h at 31° C. A culture for monitoring amino acid production was prepared by performing a 1:20 dilution of the seed culture into 10 ml of Batch Production Medium in 125 ml Erlenmeyer flasks. When appropriate, IPTG was added to a set of the cultures to induce expression of the IPTG regulated genes (final concentration 0.25 mM). Methionine fermentations were carried out for 60-66 h at 31° C. with agitation (250 rpm). For the studies reported herein, in nearly all instances, multiple transformants were fermented in parallel, and each transformant was often grown in duplicate. Most reported data points reflect the average of at least two fermentations with a representative transformant, together with control strains that were grown at the same time.

After cultivation, amino acid levels in the resulting broths were determined using liquid chromatography-mass spectrometry (LCMS). Approximately 1 ml of culture was harvested and centrifuged to pellet cells and particulate debris. A fraction of the resulting supernatant was diluted 1:5000 into aqueous 0.1% formic acid and injected in 10 μL portions onto a reverse phase HPLC column (Waters Atlantis C18, 2.1×150 mm). Compounds were eluted at a flow rate of 0.350 mL min⁻¹, using a gradient mixture of 0.1% formic acid in acetonitrile (“B”) and 0.1% formic acid in water (“A”), (1% B→50% B over 4 minutes, hold at 50% B for 0.2 minutes, 50% B→1% over 1 minute, hold at 1% for 1.8 minutes). Eluting compounds were detected with a triple-quadropole mass spectrometer using positive electrospray ionization. The instrument was operated in MRM mode to detect amino acids (lysine: 147→84 (15 eV); methionine: 150→104 (12 eV); threonine/homoserine: 120→74 (10 eV); aspartic acid: 134→88 (15 eV); glutamic acid: 148→84 (15 eV); O-acetylhomoserine: 162→102 (12 eV); and homocysteine: 136→90 (15 eV)). On occasion, additional amino acids were quantified using similar methods (e.g. homocystine, glycine, S-adenosylmethionine). Individual amino acids were quantified by comparison with amino acid standards injected under identical conditions. Using this mass spectrometric method it is not possible to distinguish between homoserine and threonine. Therefore, when necessary, samples were also derivatized with a fluorescent label and subjected to liquid chromatography followed by fluorescent detection. This method was used to both resolve homoserine and threonine as well as to confirm concentrations determined using the LCMS method.

| Seed Culture Medium for Production Assays | | |
|---|---|---|
| Glucose | 100 | g/L |
| Ammonium acetate | 3 | g/L |
| KH₂PO₄ | 1 | g/L |
| MgSO₄-7H₂O | 0.4 | g/L |
| FeSO₄-7H₂O | 10 | mg/L |
| MnSO₄-4H₂O | 10 | mg/L |
| Biotin | 50 | μg/L |
| Thiamine-HCl | 200 | μg/L |
| Soy protein | 15 | ml/L |
| Hydrolysate | | |
| (total nitrogen 7%) | | |
| Yeast extract | 5 | g/L |
| pH 7.5 | | |
| Batch Production Medium for Production Assays | | |
| Glucose | 50 | g/L |
| (NH₄)₂SO₄ | 45 | g/L |
| KH₂PO₄ | 1 | g/L |
| MgSO₄-7H₂O | 0.4 | g/L |
| FeSO₄-7H₂O | 10 | mg/L |
| MnSO₄-4H₂O | 10 | mg/L |
| Biotin | 50 | μg/L |
| Thiamine-HCl | 200 | μg/L |
| Soy protein | 15 | ml/L |
| hydrolysate | | |
| (total nitrogen 7%) | | |
| CaCO₃ | 50 | g/L |
| Cobalamin | 1 | μg/ml |
| pH 7.5 | | |

(cobalamin addition not necessary when lysine is the target aspartate-derived amino acid)

### EXAMPLE 11

### Heterologous Wild-Type and Mutant lysC Variants Increase Lysine Production in C. glutamicum and B. lactofermentum.

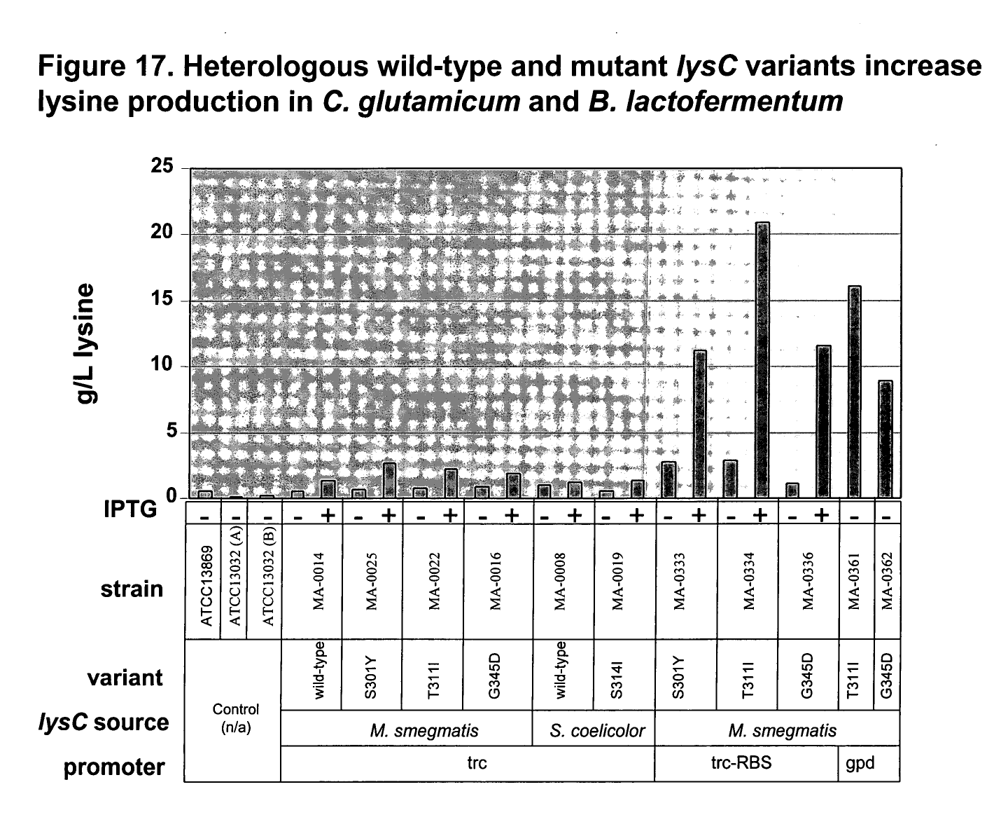

Aspartokinase is often the rate-limiting activity for lysine production in corynebacteria. The primary mechanism for regulating aspartokinase activity is allosteric regulation by the combination of lysine and threonine. Heterologous operons encoding aspartokinases and aspartate semi-aldehyde dehydrogenases were cloned from M. smegmatis and S. coelicolor as described in Example 2. Site-directed mutagenesis was used to generate deregulated alleles (see Example 3), and these modified genes were inserted into vectors suitable for expression in corynebacteria (Example 1). The resulting plasmids, and the wild-type counterparts, were transformed into strains, including wild-type C. glutamicum strain ATCC 13032 and wild-type B. lactofermentum strain ATCC 13869, which were analyzed for lysine production (FIG. 17).

Strains MA-0014, MA-0025, MA-0022, MA-0016, MA-0008 and MA-0019 contain plasmids with the MB3961 backbone (see Example 1). Increased expression, via addition of IPTG to the production medium, of either wild-type or deregulated heterologous lysC-asd operons promoted lysine production. Strain ATCC 13869 is the untransformed control for these strains. The plasmids containing M. smegmatis S301Y, T311I, and G345D alleles were most effective at enhancing lysine production; these alleles were chosen for expression for expression from improved vectors. Improved vectors containing deregulated M. smegmatis alleles were transformed into C. glutamicum (ATCC 13032) to generate strains MA-0333, MA-0334, MA-0336, MA-0361, and MA-0362 (plasmids contain either trcRBS or gpd promoter, MB4094 backbone; see Example 1). Strain ATCC 13032 (A) is the untransformed control for strains MA-0333, MA-0334 and MA-0336. Strain ATCC 13032 (B) is the untransformed control for strains MA-0361 and MA-0362.Strains MA-0333, MA-0334, MA-0336, MA-0361, and MA-0362 all displayed improvement in lysine production. For example, strain MA-0334 produced in excess of 20 g/L lysine from 50 g/L glucose. In addition, the T31 11 and G345D alleles were shown to be effective when expressed from either the trcRBS or gpd promoter.

### EXAMPLE 12

### S. coelicolor hom G362E Variant Increases Carbon Flow to Homoserine in C. glutamicum Strain, MA-0331

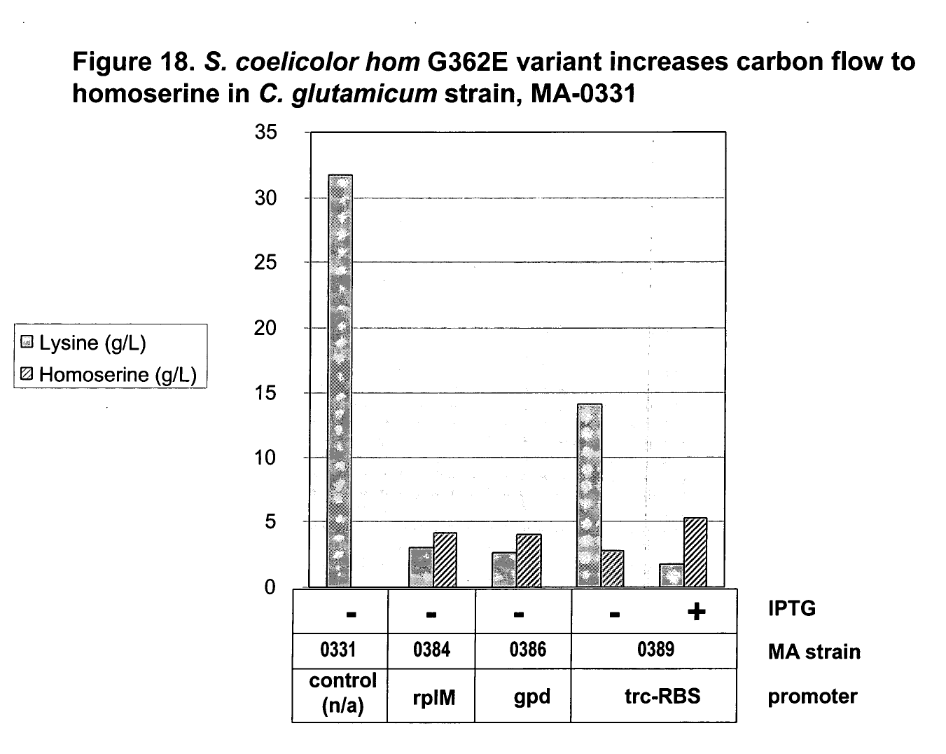

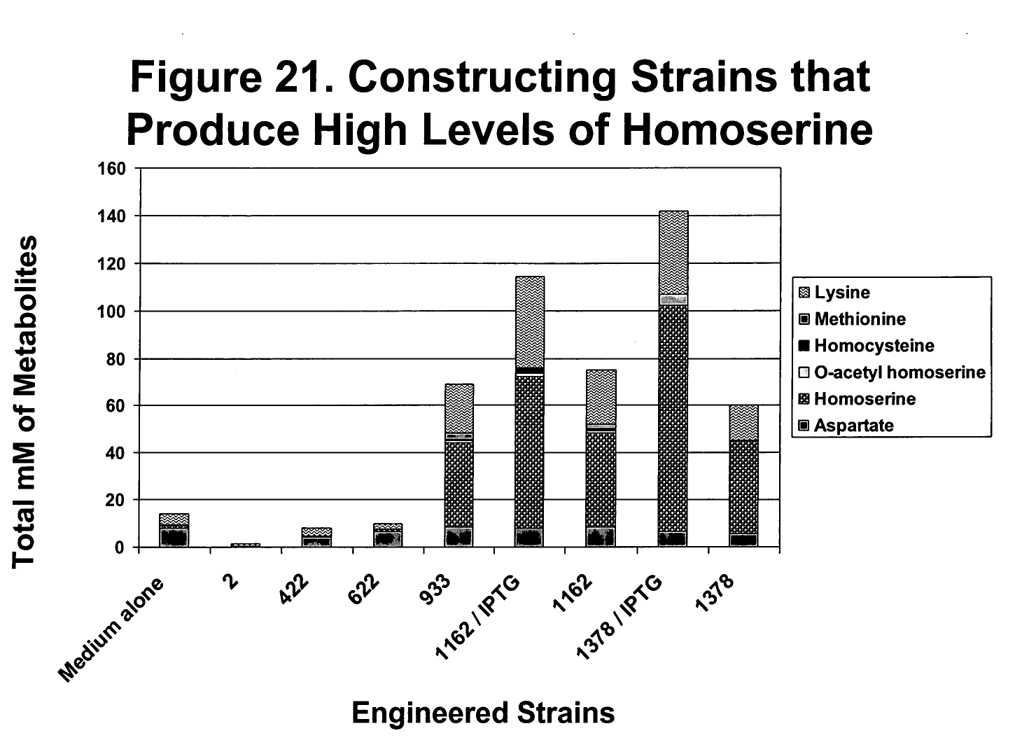

As shown in Example 11, deregulation of aspartokinase increased carbon flow to aspartate-derived amino acids. In principle, aspartokinase activity could be increased by the use of deregulated lysC alleles and/or by elimination of the small molecules that mediate the allosteric regulation (lysine or threonine). FIG. 18 (strain MA-0331) shows that high levels of lysine accumulated in the broth when the hom-thrB locus was inactivated. Hom and thrB encode for homoserine dehydrogenase and homoserine kinase, respectively, two proteins required for the production of threonine. Lysine accumulation was also observed when only the thrB gene was deleted (see strain MA-0933 in FIG. 21 (MA-0933 is one example, though it is not appropriate to directly compare MA-0933 to MA-033 1, as these strains are from different genetic backgrounds).

In order to increase carbon flow to methionine pathway intermediates, a putative deregulated variant of the S. coelicolor hom gene was transformed into MA-0331. Similar strategies were used to engineer strains containing only the thrB deletion. Strains MA-0384, MA-0386, and MA-0389 contain the S. coelicolor homG362E variant under the control of the rplM, gpd, and trcRBS promoters, respectively. These plasmids also contain an additional substitution (G43S) that was introduced as part of the site-directed mutagenesis strategy; subsequent experiments suggested that the G43S substitution does not enhance Hom activity. FIG. 18 shows the results from shake flask experiments performed using strains MA-0331, MA-0384, MA-0386, and MA-0389, in whichbroths were analyzed for aspartate-derived amino acids, including lysine and homoserine. Strains expressing the S. coelicolor homG362E gene display a dramatic decrease in lysine production as well as a significant increase in homoserine levels. Broth levels of homoserine were in excess of 5 g/L in strains such as MA-0389. It is possible that significant levels of homoserine still remain within the cell or that some homoserine has been converted to additional products. Overexpression of deregulated lysC and other genes downstream of hom, together with hom, may increase production of homoserine-based amino acids, including methionine (see below).

### EXAMPLE 13

### Heterologous Phosphoenolpyruvate Carboxylase (Ppc) Enzymes Increase Carbon Flow to Aspartate-Derived Amino Acids

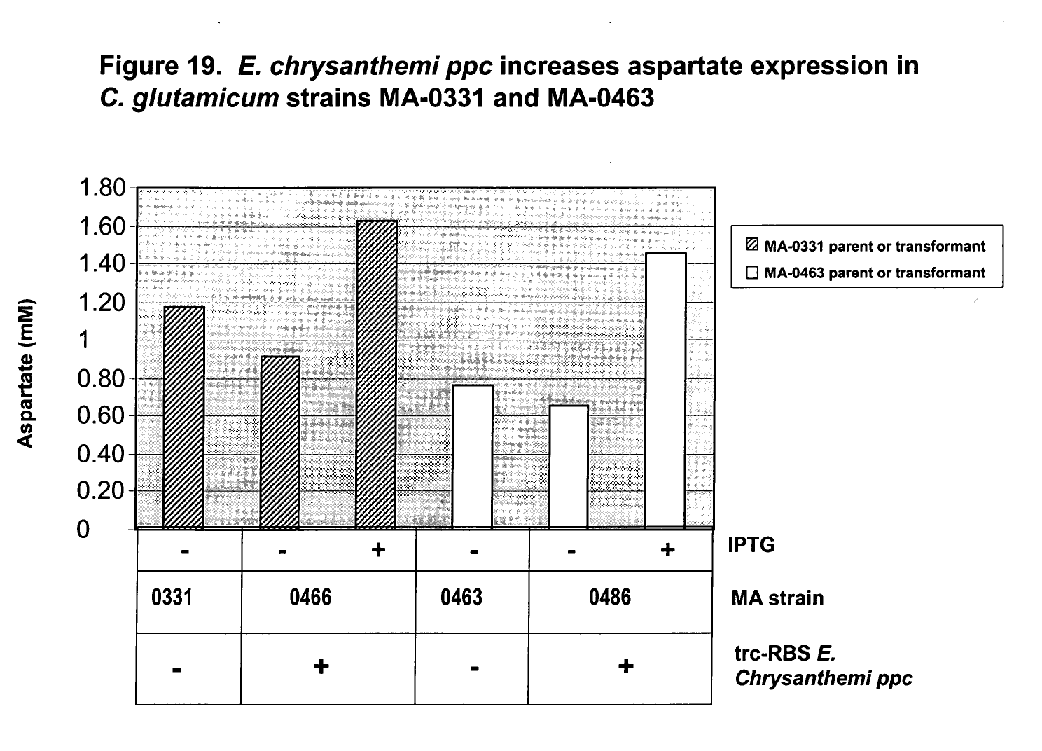

Phosphoenolpyruvate carboxylase (Ppc), together with pyruvate carboxylase (Pyc), catalyze the synthesis of oxaloacetic acid (OAA), the citric acid cycle intermediate that feeds directly into the production of aspartate-derived amino acids. The wild-type E. chrysanthemi ppc gene was cloned into expression vectors under control of the IPTG inducible trcRBS promoter. This plasmid was transformed into high lysine strains MA-033 1 and MA-0463 (FIG. 19). Strains were grown in the absence or presence of IPTG and analyzed for production of aspartate-derived amino acids, including aspartate. Strain MA-0331 contains the hom-thrBA mutation, whereas MA-0463 contains the M. smegmatis lysC (T311I)-asd operon integrated at the deleted hom-thrB locus; the lysC-asd operon is under control of the C. glutamicum gpd promoter. FIG. 19 shows that the E. chrysanthemippc gene increased the accumulation of aspartate. This difference was even detectable in strains that converted most of the available aspartate into lysine.

### EXAMPLE 14

### Heterologous Dihydrodipicolinate Synthases (dapA) Enzymes Increase Lysine Production

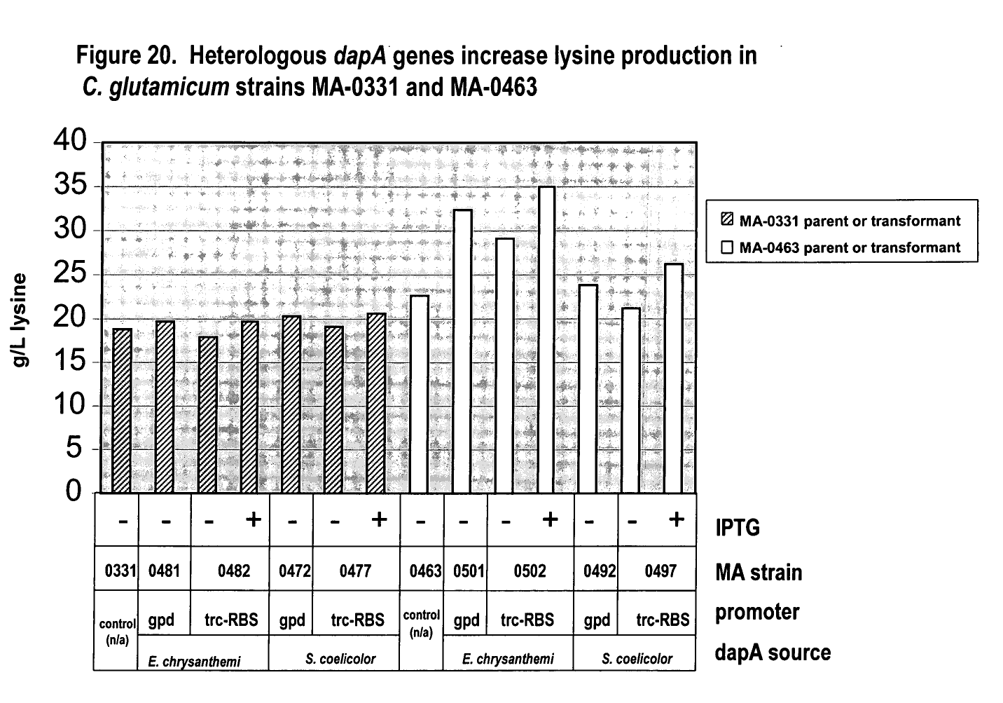

Dihydrodipicolinate synthase is the branch point enzyme that commits carbon to lysine biosynthesis rather than to the production of homoserine-based amino acids. DapA converts aspartate-B-semialdehyde to 2,3-dihydrodipicolinate. The wild-type E. chrysanthemi and S. coelicolor dapA genes were cloned into expression vectors under the control of the trcRBS and gpd promoters. The resulting plasmids were transformed into strains MA-0331 and MA-0463, two strains that had already been engineered to produce high levels of lysine (see Example 13). MA-0463 was engineered for increased expression of the M. smegmatis lysC(T311I)-asd operon. This manipulation is expected to drive production of aspartate-B-semialdehyde, the substrate for the DapA catalyzed reaction. Strains MA-0481, MA-0482, MA-0472, MA-0501, MA-0502, MA-0492, MA-0497 were grown in shake flask, and the broths were analyzed for aspartate-derived amino acids, including lysine. As shown in FIG. 20, increased expression of either the E. chrysanthemi or S. coelicolor dapA gene increases lysine production in the MA-0331 and MA-0463 backgrounds. Strain MA-0502 produced nearly 35 g/L lysine in a 50 g/L glucose process. It may be possible to engineer further lysine improvements by constructing deregulated variants of these heterologous dapA genes.

### EXAMPLE 15

### Constructing Strains that Produce High Levels of Homoserine

Strains that produce high levels of homoserine-based amino acids can be generated through a combination of genetic engineering and mutagenesis strategies. As an example, five distinct genetic manipulations were performed to construct MA-1378, a strain that produces >10 g/L homoserine (FIG. 21). To generate MA-1378, wild-type C. glutamicum was first mutated using nitrosoguanidine (NTG) mutagenesis (based on the protocol described in “A short course in bacterial genetics.” J. H. Miller. Cold Spring Harbor Laboratory Press. 1992, page 143) followed by selection of colonies that grew on minimal plates containing high levels of ethionine, a toxic methionine analog. The endogenous mcbR locus was then deleted in one of the resulting ethionine-resistant strains (MA-0422) using plasmid MB4154 in order to generate strain MA-0622. McbR is a transcriptional repressor that regulates the expression of several genes required for the production of sulfur-containing amino acids such as methionine (see Rey, D. A., Puhler, A., and Kalinowski, J., J. Biotechnology 103:51-65, 2003). In several instances we observed that inactivation of McbR generated strains with increased levels of homoserine-based amino acids. Plasmid MB4084 was utilized to delete the thrB locus in MA-0622, causing the accumulation of lysine and homoserine; methionine and methionine pathway intermediates also accumulated to a lesser degree. MA-0933 resulted from this manipulation. As described above, it is believed that the lysine and homoserine accumulation was a result of deregulation of lysC, via the lack of threonine production. In order to further optimize carbon flow to aspartate-B-semialdehyde and downstream amino acids, MA-0933 was transformed with an episomal plasmid expressing the M. smegmatis lysC (T311I)-asd operon (strain MA-162). High homoserine producing strain MA-1 162 was then mutagenized with NTG, and colonies were selected on minimal medium plates containing a level of methionine methylsulfonium chloride (MMSC) that is normally inhibitory to growth. MA-1378 was one such MMSC-resistant strain.

### EXAMPLE 16

### Heterologous Homoserine Acetyltransferases (MetA) Enzymes Increase Carbon Flow to Homoserine-Based Amino Acids

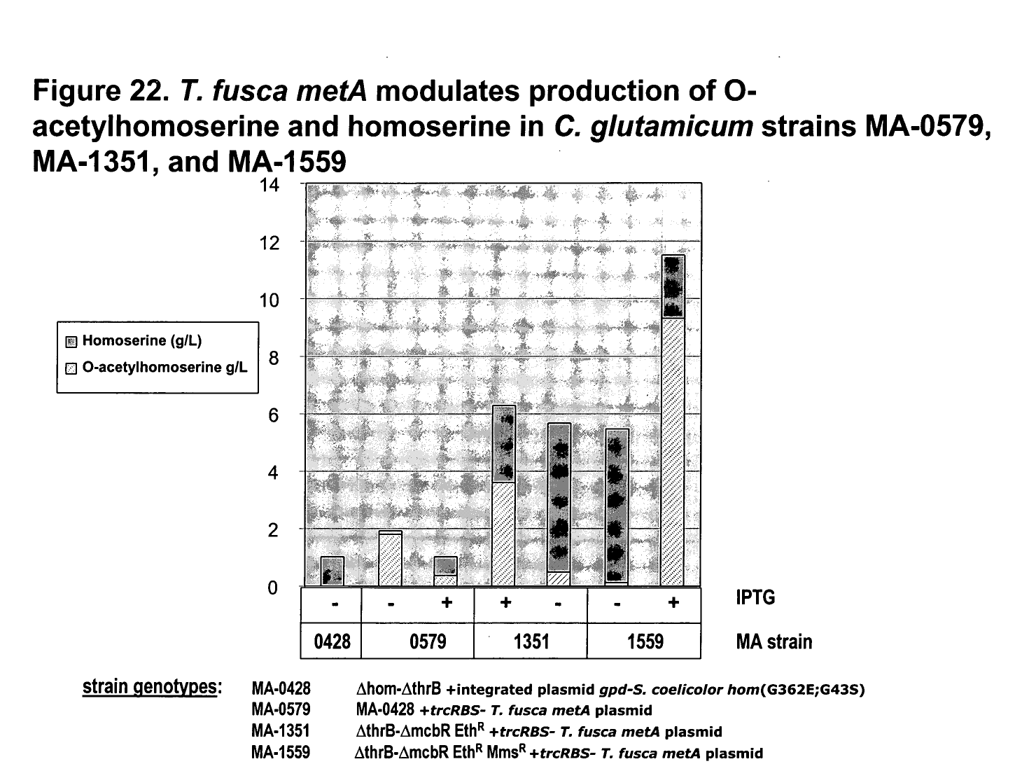

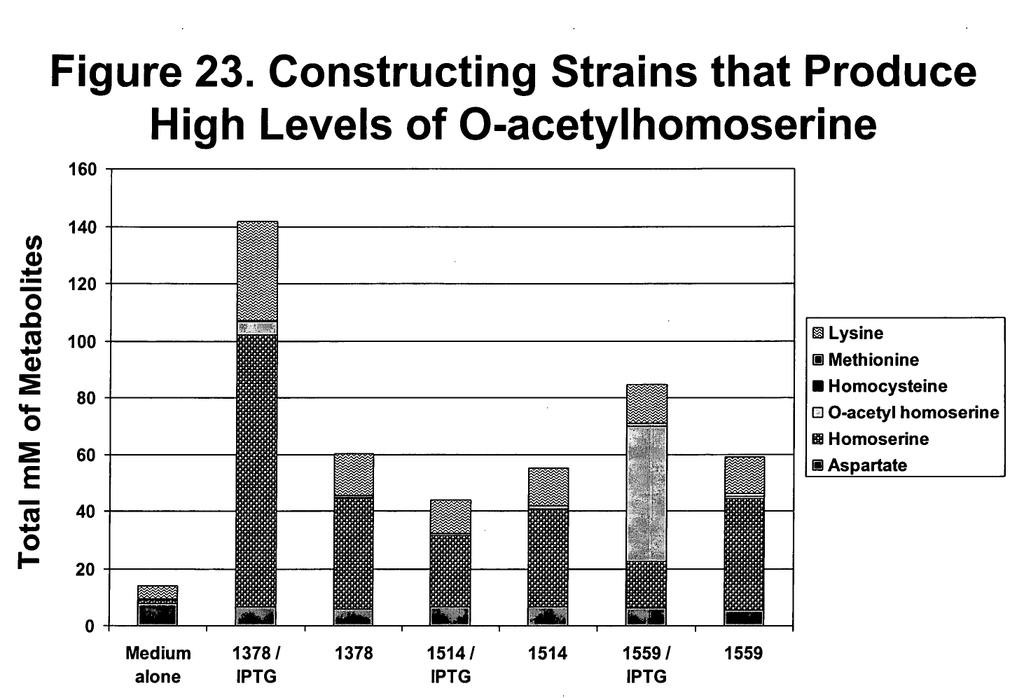

MetA is the commitment step to methionine biosynthesis. The wild-type T. fusca metA gene was cloned into an expression vector under the control of the trcRBS promoter. This plasmid was transformed into high homoserine producing strains to test for elevated MetA activity (FIGS. 22 and 23). MA-0428, MA-0933, and MA-1514 were example high homoserine producing strains. MA-0428 is a wild-type ATCC 13032 derivative that has been engineered with plasmid MB4192 (see Example 1) to delete the hom-thrB locus and integrate the gpd-S. coelicolor hom(G362E) expression cassette. MA-1514 was constructed by using novobiocin to allow for loss of the M. smegmatis lysC(T311I)-asd operon plasmid from strain MA-1378. This manipulation was performed to allow for transformation with the episomal plasmid containing the T. fusca metA gene and the kanR selectable marker. Strain MA-1559 resulted from the transformation of strain MA-1514 with the T. fusca metA gene under control of the trcRBS promoter. MA-0933 is as described in Example 15. Induction of T. fusca metA expression in each of these high homoserine strains resulted in accumulation of O-acetylhomoserine in culture broths. For example, strain MA-1559 displayed OAH levels in excess of 9 g/L. Additional manipulations can be performed to elicit conversion of OAH to other products, including methionine.

### EXAMPLE 17

### Effects of metA Variants on Methionine Production in C. glutamicum

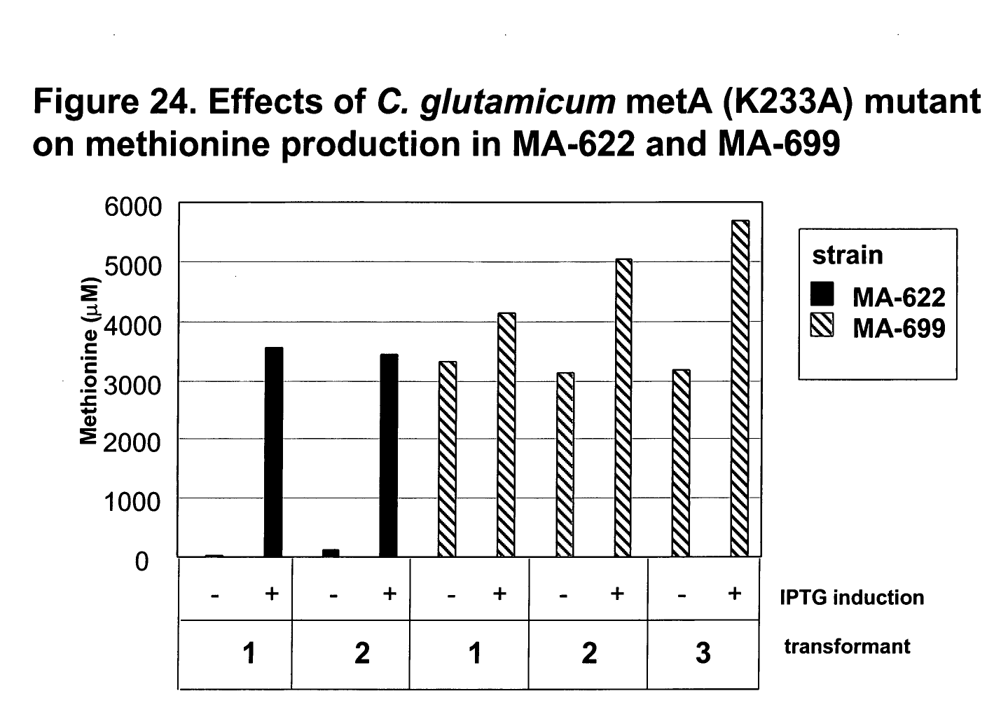

C. glutamicum homoserine acetyltransferase (MetA) variants were generated by site-directed mutagenesis of MetA-encoding DNA (Example 6). C. glutamicum strains MA-0622 and MA-0699 were transformed with a high copy plasmid, MB4236, that encodes MetA with a lysine to alanine mutation at position 233 (MetA (K233A)). This plasmid also contains a wild-type copy of the C. glutamicum metY gene. Strain MA-0699 was constructed by transforming MA-0622 with plasmid MB4192 to delete the hom-thrB locus and integrate the gpd- S. coelicolor hom(G362E) expression cassette. metA and metYare expressed in a synthetic metAY operon under control of a modified version of the trc promoter. The strains were cultured in the presence and absence of IPTG induction, and methionine productivity was assayed. Methionine production from each strain is plotted in FIG. 24. As shown, individual transformants of MA-622 and MA-699, when cultured under inducing conditions, each produced over 3000 μM methionine. MA-699 strains, which express an S. coelicolor hom G362E variant under the control of a constitutive promoter, produced over 3000 μM methionine in the absence of IPTG. IPTG induction resulted in an increased methionine production by 1000-2500 μM. These data show that expression of MetA (K233A) enhances methionine production. Manipulation of methionine biosynthesis at multiple points can further enhance production.

### EXAMPLE 17

### Effects of metY Variants on Methionine Production in C. glutamicum

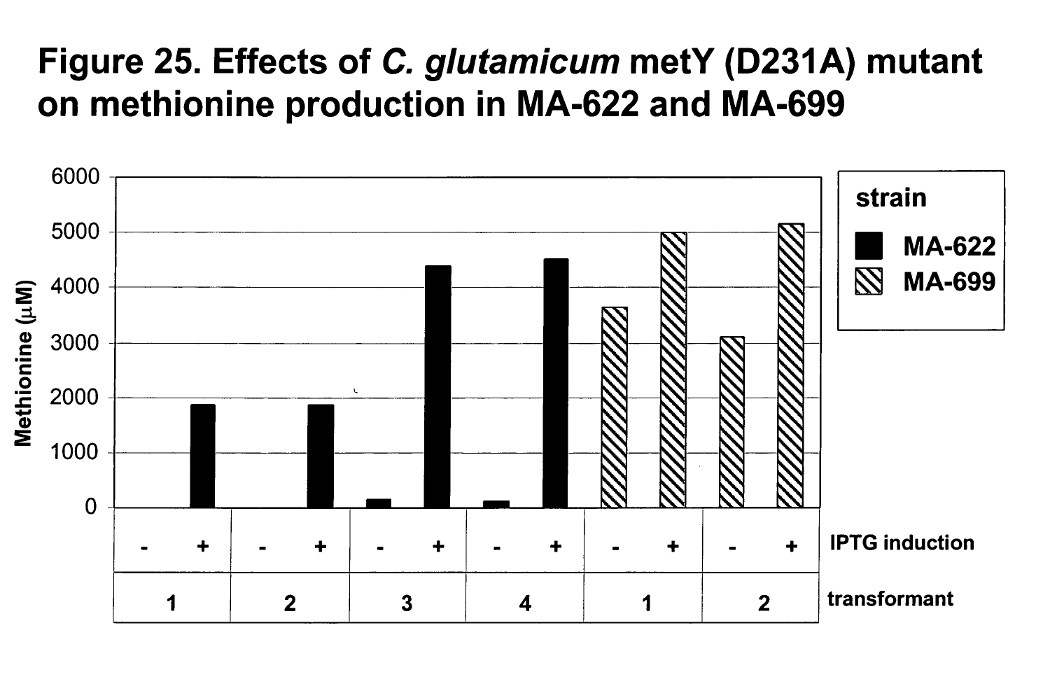

C. glutamicum O-acetylhomoserine sulfhydrylase (MetY) variants were generated by site-directed mutagenesis of MetY-encoding DNA (Example 6). C. glutamicum strain MA-622 and strain MA-699 were transformed with a high copy plasmid, MB4238, that encodes MetY with an aspartate to alanine mutation at position 231 (MetY (D231A)). This plasmid also contains the wild-type copy of the C. glutamicum metA gene, expressed as in Example 16. The strains were cultured in the presence and absence of IPTG induction, and methionine productivity was assayed. The methionine production from each strain is plotted in FIG. 25. As shown, individual transformants of MA-622, when cultured under conditions in which expression of MetY (D231A) was induced, each produced over 1800 μM methionine. MA-622 strains showed variation in the levels of methionine produced by individual transformants (i.e., transformants 1 and 2 produced approx. 1800 μM methionine when induced, whereas transformants 3 and 4 produced over 4000 μM methionine when induced). MA-699 strains, which express an S. coelicolor Hom variant, produced approximately 3000 μM methionine in the absence of IPTG. IPTG induction increased methionine production by 1500-2000 μM. These data show that expression of MetY (D231A) enhances methionine production. Methionine production was also enhanced in strain MA-699, relative to MA-622. Expression of MetY (D231A) in strain MA-699 further enhanced methionine production in that strain.

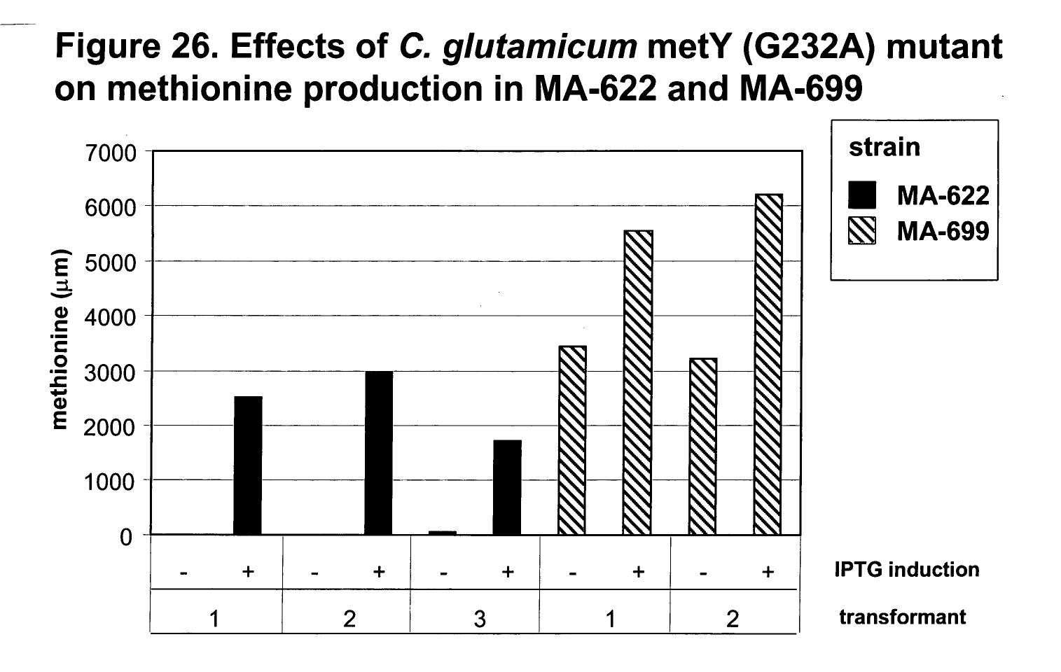

A second variant allele of metY was expressed in C. glutamicum and assayed for its effect on methionine production. C. glutamicum strain MA-622 and strain MA-699 were transformed with a high copy plasmid, MB4239, that encodes MetY with a glycine to alanine mutation at position 232 (MetY (G232A)). The strains were cultured in the presence and absence of IPTG induction, and methionine productivity was assayed. The methionine production from each strain is plotted in FIG. 26. As shown, individual transformants of MA-622, when cultured under conditions in which expression of MetY (G232A) was induced, each produced over 1700 μM methionine. MA-699 strains produced approximately 3000 μM methionine in the absence of IPTG. IPTG induction resulted in an increased methionine production by 2000-3000 μM. These data show that expression of MetY (G232A) enhances methionine production. Methionine production was also enhanced in strain MA-699, relative to MA-622. Expression of MetY (G232A) in strain MA-699 further enhanced methionine production in that strain.

### EXAMPLE 18

### Methionine Production in C. glutamicum Strains Expressing metA and metY Wild-Type and Mutant Alleles

Methionine production was assayed in five different C. glutamicum strains. Four of these strains express a unique combination of episomal C. glutamicum metA and metY alleles, as listed in Table 14. A fifth strain, MA-622, does not contain episomal metA or metY alleles. The amount of methionine produced by each strain (g/L) is listed in Table 14.

The highest levels of methionine production were observed in strains expressing a combination of either a wild-type metA and a variant metY, or a wild-type metY and a variant metA.

TABLE 14
Methionine production in strains expressing
C. glutamicum metA and metY wild-type and mutant alleles

| | | | | methionine |
|---|---|---|---|---|
| Strain | IPTG | metA allele | metY allele | (g/L) |
| MA-622 | − | None | none | 0.00 |
| MA-641 | − | WT | WT | 0.03 |
| MA-721 | − | K233A | WT | 0.00 |
| MA-721 | + | K233A | WT | 0.53 |
| MA-725 | − | WT | D231A | 0 |
| MA-725 | + | WT | D231A | 0.28 |
| MA-727 | − | WT | G232A | 0 |
| MA-727 | + | WT | G232A | 0.37 |

### EXAMPLE 19

### Combinations of Genetic Manipulations, Using Both Heterologous and Native Genes, Elicits Production of Aspartate-Derived Amino Acids

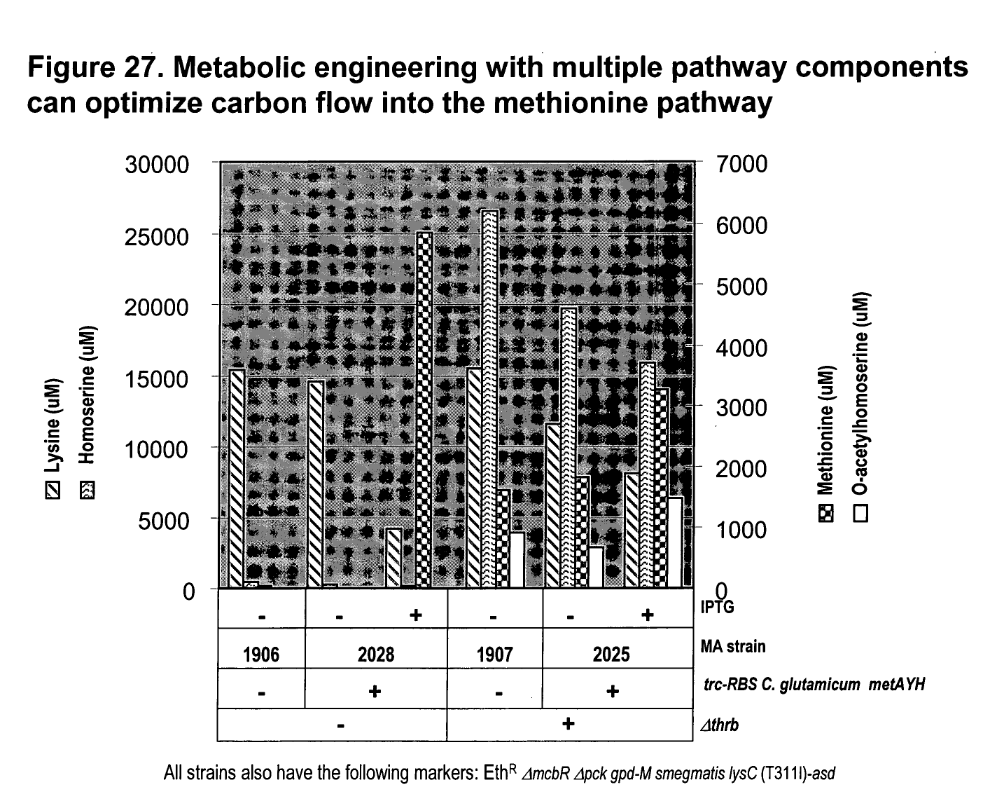

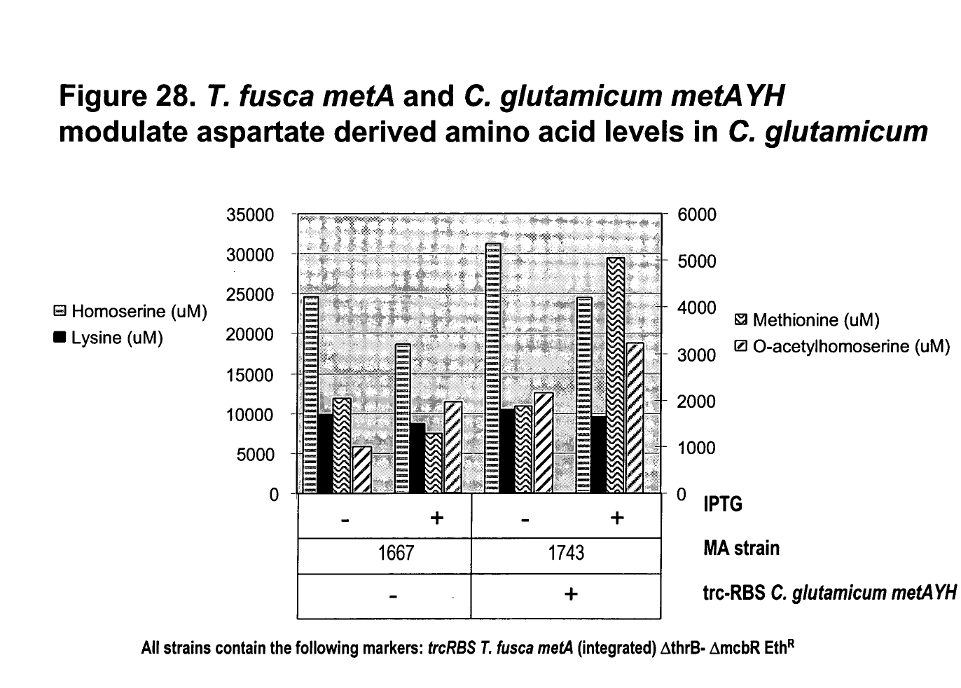

As described above, gene combinations may optimize corynebacteria for the production of aspartate-derived amino acids. Below are examples that show how multiple manipulations can increase the production of methionine. FIG. 27 shows the production of several aspartate-derived amino acids by strains MA-2028 and MA-2025 along with titers from their parent strains MA-1906 and MA-1907, respectively. MA-1906 was constructed by using plasmid MB4276 to delete the native pck locus in MA-0622 and replace pck with a cassette for constitutive expression of the M. smegmatis lysC(T311I)-asd operon. MA-1907 was generated by similar transformation of MB4276 into MA-0933. MA-2028 and MA-2025 were constructed by transformation of the respective parents with MB4278, an episomal plasmid for inducible expression of a synthetic C. glutamicum metA YH operon (see Example 3). Parent strains MA-1906 and MA-1907 produce lysine or lysine and homoserine, respectively; methionine and methionine pathway intermediates are also produced by these strains. The scale for lysine and homoserine is on the left y-axis; the scale for methionine and O-acetylhomoserine is on the right y-axis. With IPTG induction, MA-2028 showed a decrease in lysine levels and an increase in methionine levels. MA-2025 also displayed an IPTG-dependent decrease in lysine production, together with increased production of methionine and O-acetylhomoserine. Strain MA-1743 is another example of how combinatorial engineering can be employed to generate strains that produce methionine. MA- 1743 was generated by transformation of MA-1667 with metAYHexpression plasmid MB4278. MA-1667 was constructed by first engineering strain MA-0422 (see Example 15) with plasmid MB4084 to delete thrB, and next using plasmid MB4286 to both delete the mcbR locus and replace mcbR with an expression cassette containing trcRBS-T. fusca metA. In this example and in other examples where trcRBS has been integrated at single copy, expression does not appear to be as tightly regulated as seen with the episomal plasmids (as judged by amino acid production). Thismay be due to decreased levels of the laclq inhibitor protein. IPTG induction of strain MA- 1743 elicits production of methionine and pathway intermediates, including O-acetylhomoserine (FIG. 28; the scale for lysine and homoserine is on the left y-axis; the scale for methionine and O-acetylhomoserine is on the right y-axis).

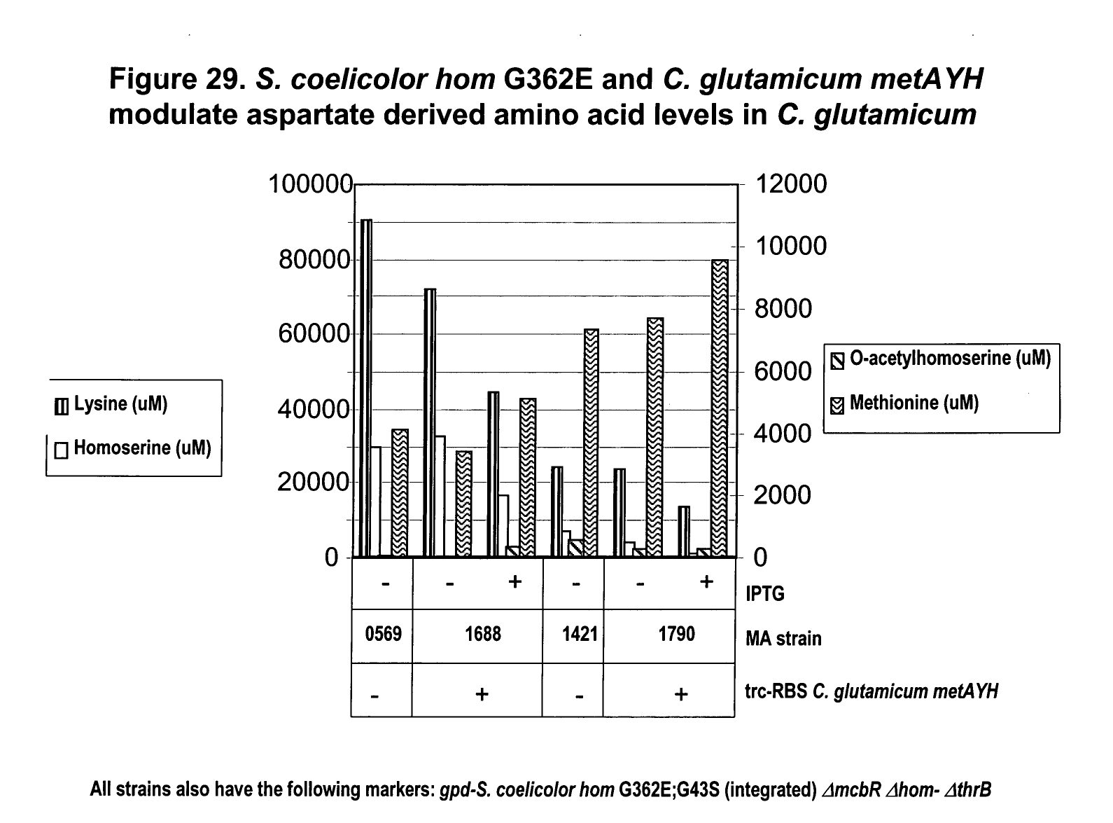

Strains MA-1688 and MA-1790 are two additional strains that were engineered with multiple genes, including the MB4278 metAYH expression plasmid (see FIG. 29; the scale for lysine and homoserine is on the left y-axis; the scale for methionine and O-acetylhomoserine is on the right y-axis). Transforming MA-0569 with MB4278 generated MA-1688. MA-0569 was constructed by sequentially using MB4192 and MB4165 to first delete the hom-thrB locus and integrate the gpd-S. coelicolor hom(G362E) expression cassette and then delete mcbR. MA-1790 construction required several steps. First, a NTG mutant derivative of MA-0428 was identified based on its ability to allow for growth of a Salmonella metE mutant. In brief, a population of mutagenized MA-0428 cells was plated onto a minimal medium containing threonine and a lawn (>106 cells of the Salmonella metE mutant). The Salmonella metE mutant requires methionine for growth. After visual inspection, the corynebacteria colonies (e.g. MA-0600) surrounded by a halo of Salmonella growth were isolated and subjected to shake flask analysis. Strain MA-600 was next mutagenized to ethionine resistance as described above, and one resulting strain was designated MA-0993. The mcbR locus was then deleted from MA-0993 using plasmid MB4165, and MA-1421 was the product of this manipulation. Transformation of MA-1421 with MB4278 generated MA-1 790. FIG. 29 shows that IPTG induction stimulates methionine production in both MA-1688 and MA-1790, and decreases in lysine and homoserine titers.

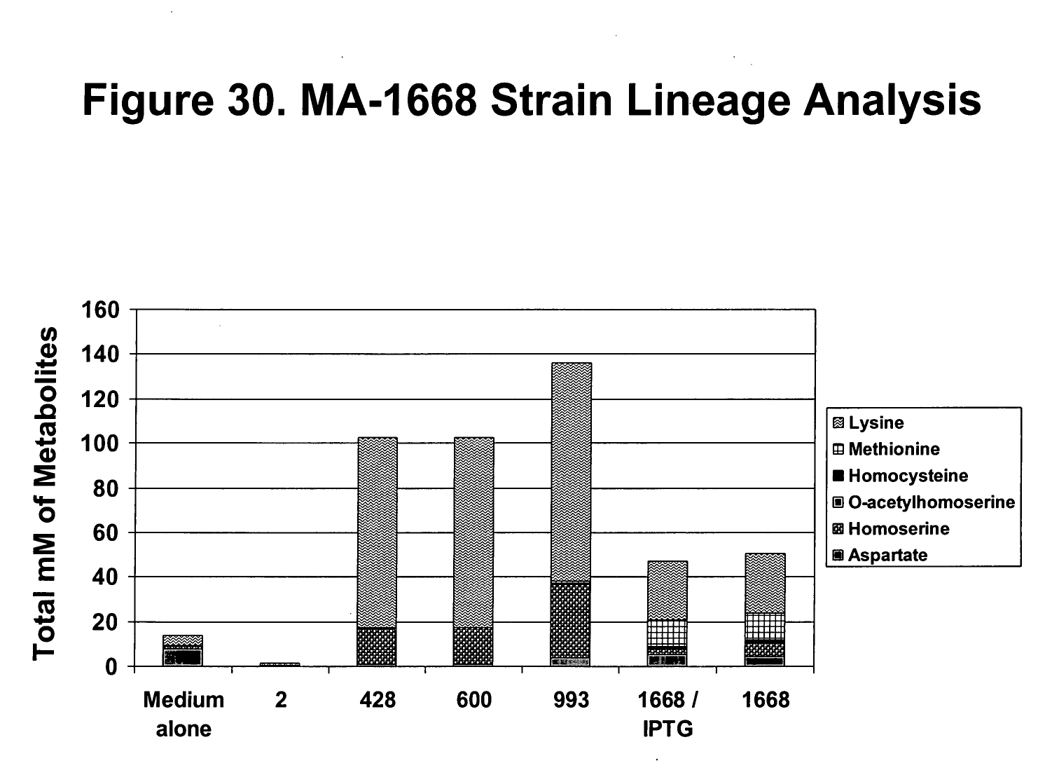

FIG. 30 shows the metabolite levels of strain MA-1668 and its parent strains. The scale for lysine and homoserine is on the left y-axis; the scale for methionine and O-acetylhomoserine is on the right y-axis. Strain MA-1668 was generated by transformation of MA-0993 with plasmid MB4287. Manipulation with MB4287 results in deletion of the mcbR locus and replacement with C. glutamicum metA(K233A)-metB. Strain MA-1668 produces approximately 2 g/L methionine, with decreased levels of lysine and homoserine relative to its progenitor strains. Strain MA-1 668 is still amenable to further rounds of molecular manipulation.

Table 15 lists the strains used in these studies. The ‘::’ nomenclature indicates that the expression construct following the ‘::’ is integrated at the named locus prior to the ‘::’. EthR6 and EthR10 represent independently isolated ethionine resistant mutants. The Mcf3 mutation confers the ability to enable a Salmonella metE mutant to grow (see example 19). The Mms13 mutation confers methionine methylsulfonium chloride resistance (see example 15).

TABLE 15
Strains used in studies described herein.

| Name | Strain Genotype |
|---|---|
| MA-0002 | is ATCC 13032 |
| MA-0003 | is ATCC 13869 |
| MA-0008 | lacIq-trc-S. coelicolor lysC-asd(A191V) (episomal) |
| MA-0014 | lacIq-trc-M. smegmatis lysC-asd (episomal) |
| MA-0016 | lacIq-trc-M. smegmatis lysC (G345D)-asd (episomal) |
| MA-0019 | lacIq-trc-S. coelicolor lysC (S314I)-asd (A191V) (episomal) |
| MA-0022 | lacIq-trc-M. smegmatis lysC (T311I)-asd (episomal) |
| MA-0025 | lacIq-trc-M. smegmatis lysC (S301Y)-asd (episomal) |
| MA-0331 | Δhom-ΔthrB |
| MA-0333 | lacIq-trcRBS-M. smegmatis lysC (S301Y)-asd (episomal) |
| MA-0334 | lacIq-trcRBS-M. smegmatis lysC (T311I)-asd (episomal) |
| MA-0336 | lacIq-trcRBS-M. smegmatis lysC (G345D)-asd (episomal) |
| MA-0361 | gpd-M. smegmatis lysC (T311I)-asd (episomal) |
| MA-0362 | gpd-M. smegmatis lysC (G345D)-asd (episomal) |
| MA-0384 | Δhom-ΔthrB + rplM-S. coelicolor hom (G362E; G43S) (episomal) |
| MA-0386 | Δhom-ΔthrB + gpd-S. coelicolor hom (G362E; G43S) (episomal) |
| MA-0389 | Δhom-ΔthrB + lacIq-trcRBS-S. coelicolor hom (G362E; G43S; K19N) (episomal) |
| MA-0422 | EthR6 |
| MA-0428 | Δhom-ΔthrB::gpd-S. coelicolor hom (G362E; G43S) |
| MA-0442 | Δhom-ΔthrB + gpd-S. coelicolor hom (G362E; G43S) + lacIq-trcRBS-C. |
| | glutamicum metA-RBS-C. glutamicum metY (episomal) |
| MA-0449 | Δhom-ΔthrB + gpd-S. coelicolor hom (G362E; G43S) + lacIq-trcRBS-C. |
| | glutamicum metY-RBS-C. glutamicum metA (episomal) |
| MA-0456 | Δhom-ΔthrB::gpd-S. coelicolor hom (G362E; G43S) + gpd-T. fusca metY-RBS-T. |
| | fusca metA (episomal) |
| MA-0463 | Δhom-ΔthrB::gpd-M. smegmatis lysC (T311I)-asd |
| MA-0466 | Δhom-ΔthrB + lacIq-trcRBS-E. chrysanthemi ppc (episomal) |
| MA-0472 | Δhom-ΔthrB + gpd-S. coelicolor dapA (episomal) |
| MA-0477 | Δhom-ΔthrB + lacIq-trcRBS-S. coelicolor dapA (episomal) |
| MA-0481 | Δhom-ΔthrB + gpd-E. chrysanthemi dapA (episomal) |
| MA-0482 | Δhom-ΔthrB + lacIq-trcRBS-E. chrysanthemi dapA (episomal) |
| MA-0486 | Δhom-ΔthrB::gpd-M. smegmatis lysC (T311I)-asd + lacIq-trcRBS-E. |
| | chrysanthemi ppc (episomal) |
| MA-0492 | Δhom-ΔthrB::gpd-M. smegmatis lysC (T311I)-asd + gpd-S. coelicolor dapA |
| | (episomal) |
| MA-0497 | Δhom-ΔthrB::gpd-M. smegmatis lysC (T311I)-asd + lacIq-trcRBS-S. coelicolor |
| | dapA (episomal) |
| MA-0501 | Δhom-ΔthrB::gpd-M. smegmatis lysC (T311I)-asd + gpd-E. chrysanthemi dapA |
| | (episomal) |
| MA-0502 | Δhom-ΔthrB::gpd-M. smegmatis lysC (T311I)-asd + lacIq-trcRBS-E. |
| | chrysanthemi dapA (episomal) |
| MA-0569 | ΔmcbR + Δhom-ΔthrB::gpd-S. coelicolor hom (G362E; G43S) |
| MA-0570 | Δhom-ΔthrB + gpd-S. coelicolor hom (G362E; G43S) + lacIq-trcRBS-T. fusca |
| | metY-RBS-T. fusca metA (episomal) |
| MA-0578 | Δhom-ΔthrB + gpd-S. coelicolor hom (G362E; G43S) + gpd-T. fusca metA |
| | (episomal) |
| MA-0579 | Δhom-ΔthrB + gpd-S. coelicolor hom (G362E; G43S) + lacIq-trcRBS-T. fusca |
| | metA (episomal) |
| MA-0600 | Δhom-ΔthrB + gpd-S. coelicolor hom (G362E; G43S) + Mcf3 |
| MA-0622 | ΔmcbR + EthR6 |
| MA-0641 | ΔmcbR + EthR6 + gpd-C. glutamicum metA-RBS-C. glutamicum metY (episomal) |
| MA-0699 | ΔcbR + EthR6 + Δhom-ΔthrB::gpd-S. coelicolor hom (G362E) |
| MA-0721 | ΔmcbR + EthR6 + lacIq-trcRBS-C. glutamicum metA (K233A)-RBS-C. |
| | glutamicum metY (episomal) |
| MA-0725 | ΔmcbR + EthR6 + lacIq-trcRBS-C. glutamicum metA-RBS-C. glutamicum metY |
| | (D231A) (episomal) |
| MA-0727 | ΔmcbR + EthR6 + lacIq-trcRBS-C. glutamicum metA-RBS-C. glutamicum metY |
| | (G232A) (episomal) |
| MA-0933 | ΔthrB + ΔmcbR + EthR6 |
| MA-0993 | Δhom-ΔthrB::gpd-S. coelicolor hom (G362E; G43S) + Mcf3 + EthR10 |
| MA-1162 | ΔthrB + ΔmcbR + EthR6 + lacIq-trcRBS-M. smegmatis lysC (T311I)-asd (episomal) |
| MA-1351 | ΔthrB + ΔmcbR + EthR6 + lacIq-trcRBS-T. fusca metA (episomal) |
| MA-1378 | ΔthrB + ΔmcbR + EthR6 + Mms13 + lacIq-trcRBS-M. smegmatis lysC (T311I)-asd |
| MA-1421 | Δhom-ΔthrB::gpd S. coelicolor hom (G362E; G43S) + ΔmcbR + Mcf3 + EthR10 |
| MA-1514 | ΔthrB + ΔmcbR + EthR6 + Mms13 |
| MA-1559 | ΔthrB + ΔmcbR + EthR6 + Mms13 + lacIq-trcRBS-T. fusca metA (episomal) |
| MA-1667 | ΔthrB + EthR6 + ΔmcbR::lacIq-trcRBS-T. fusca metA (episomal) |
| MA-1668 | Δhom-ΔthrB::gpd-S. coelicolor hom (G362E; G43S) + ΔmcbR::lacIq-trcRBS- |
| | C. glutamicum metA (K233A)-RBS-C. glutamicum metB + Mcf3 + EthR10 |
| MA-1688 | ΔmcbR + Δhom-ΔthrB::gpd-S. coelicolor hom (G362E; G43S) + lacIq-trcRBS-C. |
| | glutamicum metA-RBS-C. glutamicum metY-RBS-C. glutamicum metH |
| | (episomal) |
| MA-1743 | ΔthrB + ΔmcbR::lacIq-trcRBS-T. fusca metA + EthR6 + lacIq-trcRBS-C. |
| | glutamicum metA-RBS-C. glutamicum metY-RBS-C. glutamicum metH |
| | (episomal) |
| MA-1790 | Δhom-ΔthrB::gpd-S. coelicolor hom |
| | (G362E; G43S) + ΔmcbR + Mcf3 + EthR10 + lacIq-trcRBS-C. glutamicum metA- |
| | RBS-C. glutamicum-metY-RBS-C. glutamicum-metH (episomal) |
| MA-1906 | ΔmcbR + EthR6 + Δpck::gpd-M. smegmatis lysC (T311I)-asd |
| MA-1907 | ΔmcbR + EthR6 + Δpck::gpd-M. smegmatis lysC (T311I)-asd + ΔthrB |
| MA-2025 | ΔmcbR + EthR6 + Δpck::gpd-M. smegmatis lysC (T311I)-asd + ΔthrB + lacIq- |
| | trcRBS-C. glutamicum metA-RBS-C. glutamicum metY-RBS-C. glutamicum |
| | metH (episomal) |
| MA-2028 | ΔmcbR + EthR6 + Δpck::gpd-M. smegmatis lysC (T311I)-asd + lacIq-trcRBS-C. |
| | glutamicum metA-RBS-C. glutamicum metY-RBS-C. glutamicum metH |
| | (episomal) |

TABLE 16

Amino acid sequences of exemplary heterologous proteins for amino acid
production in Escherichia coli and coryneform bacteria.
The NC number under the Gene column corresponds to the
Genbank ® protein record for the corresponding Corynebacterium
glutamicum gene.

| | | | | |
|---|---|---|---|---|
| | | GenBank ® | | SEQ |
| Gene | Organism | Protein ID | Amino Acid Sequence | ID NO: |

| | | | | |
|---|---|---|---|---|
| lysC | Mycobacterium | CAA78984 | MALVVQKYGGSSVADAERIRRVAERIVETKKAGNDVVVVVSA | 1 |
| | smegmatis | | MGDTTDDLLDLARQVSPAPPPREMDMLLTAGERISNALVAMA | |
| | | | IESLGAQARSFTGSQAGVITTGTHGNAKIIDVTPGRLRDALD | |
| | | | EGQIVLVAGFQGVSQDSKDVTTLGRGGSDTTAVAVAAALDAD | |
| | | | VCEIYTDVDGIFTADPRIVPNARHLDTVSFEEMLEMAACGAK | |
| | | | VLMLRCVEYARRYNVPIHVRSSYSDKPGTIVKGSIEDIPMED | |
| | | | AILTGVAHDRSEAKVTVVGLPDVPGYAAKVFRAVAEADVNID | |
| | | | MVLQNISKIEDGKTDITFTCARDNGPRAVEKLSALKSEIGFS | |
| | | | QVLYDDHIGKVSLIGAGMRSHPGVTATFCEALAEAGINIDLI | |
| | | | STSEIRISVLIKDTELDKAVSALHEAFGLGGDDEAVVY | |
| | | | AGTGR | |
| lysC | Amycolatopsis | AAD49567 | MALVVQKYGGSSLESADRIKRVAERIVATKKAGNDVVVVCSA | 2 |
| | mediterranei | | MGDTTDELLDLAQQVNPAPPEREMDMLLTAGERISNSLVAMA | |
| | | | IAAQGAEAWSFTGSQAGVVTTSVHGNARIIDVTPSRVTEALD | |
| | | | QGYIALVAGFQGVAQDTKDITTLGRGGSDTTAVALAAALNAD | |
| | | | VCEIYSDVDGVYTADPRVVPDAKKLDTVTYEEMLELAASGSK | |
| | | | ILHLRSVEYARRYGVPIRVRSSYSDKPGTTVTGSIEEIPVEQ | |
| | | | ALITGVAHDRSEAKITVTGVPDHTGAAARIFRVIADAEIDID | |
| | | | MVLQNVSSTVSGRTDITFTLSKANGAKAVKELEKVQAEIGFE | |
| | | | SVLYDDHVGKVSVVGAGMRSHPGVTATFCEALAEAGVNIEII | |
| | | | NTSEIRISVLIRDAQLDDAVRAIHEAFELGGDEEAVV | |
| | | | YAGSGR | |
| lysC | Streptomyces | CAB45482 | MGLVVQKYGGSSVADAEGIKRVAKRIVEAKKNGNQVVAVVSA | 3 |
| | coelicolor | | MGDTTDELIDLAEQVSPIPAGRELDMLLTAGERISMALLAMA | |
| | | | IKNLGHEAQSFTGSQAGVITDSVHNKARIIDVTPGRIRTSVD | |
| | | | EGNVAIVAGFQGVSQDSKDITTLGRGGSDTTAVALAAALDAD | |
| | | | VCEIYTDVDGVFTADPRVVPKAKKIDWISFEDMLELAASGSK | |
| | | | VLLHRCVEYARRYNIPIHVRSSFSGLQGTWVSSEPIKQGEKH | |
| | | | VEQALISGVAHDTSEAKVTVVGVPDKPGEAAAIFRAIADAQV | |
| | | | NIDMVVQNVSAASTGLTDISFTLPKSEGRKAIDALEKNRPGI | |
| | | | GFDSLRYDDQIGKISLVGAGMKSNPGVTADFFTALSDAGVNI | |
| | | | ELISTSEIRISVVTRKDDVNEAVRAVHTAFGLDSDSDEAVVY | |
| | | | GGTGR | |
| lysC | Thermobifida | ZP_00057166 | MNLRSLDWLVDYREPDSSGAPTVALIVQKYGGSSVADADAIK | 4 |
| | fusca | | RVAERIVAQKKAGYDVVVVVSAMGDTTDELLDLAKQVSPLPP | |
| | | | GRELDMLLTAGERISMALVAMAIGNLGYEARSFTGSQAGVIT | |
| | | | TSLHGNAKIIDVTPGRIRDALAEGAICIVAGFQGVSQDSKDI | |
| | | | TTLGRGGSDTTAVALAAALNADLCEIYTDVDGVFTADPRIVP | |
| | | | SARRIPQISYEEMLEMAASGAKILHLRCVEYARRYNIPLNVR | |
| | | | SSFSQKPGTWVVSEVEETEGMEQPIISGVAHDRSEAKITVVG | |
| | | | VPDRVGEAAAIFKALADAEINVDMIVQNVSAASTSRTDISFT | |
| | | | LPADSGQNALAALKKIQDKVGFESLLYNDRIGKVSLIGAGMR | |
| | | | SYPGVTARFFDAVAREGINIEMISTSEIRISIVVAQDDVDAA | |
| | | | VAAAHREFQLDADQVEAVVYGGTGR | |
| lysC | Erwinia | | MSANTDNSLIIAKFGGTSVADFDAMNRSADIVLSDAQVRVVV | 5 |
| | chrysenthemi | | LSASAGVTNLLVALAEGLPPSERTAQLEKLRQTQYAIIDRLN | |
| | | | QPAVIREEIDRMLDNVARLSEAAALATSNALTDELVSHGELI | |
| | | | STLLFVEILRERNVAAEWFDVRKIMRTNDRFGRAEPDCDALG | |
| | | | ELTRSQLTPRLAQGLIITQGFIGSEAKGRTTTLGRGGSDYTA | |
| | | | ALLGEALHASRIDIWTDVPGIYTTDPRVVPSAHRIDQITFEE | |
| | | | AAEMATFGAKVLHPATLLPAVRSDIPVFVGSSKDPAAGGTLV | |
| | | | CNNTENPPLFPALALRRKQTLLTLHSLNNLHARGFLAEVFSI | |
| | | | LARHNISVDLITTSEVNVALTLDTTGSTSTGDSLLSSALLTE | |
| | | | LSSLCRVEVEENMSLVALIGNQLSQACGVGKEVFGVLEPFNI | |
| | | | RLICYGASSHNLCFLVPSSDAEQVVQTLHHNLFE | |
| lysC | Shewanella | AAN56424 | MLEKRKLSGSKLFVKKFGGTSVGSIERIEVVAEQIAKSAHSG | 6 |
| | oneidensis | | EQQVLVLSAMAGETNRLFALAAQIDPPASARELDMLVSTGEQ | |
| | | | ISIALMAMALQRRGIKARSLTGDQVQIHTNSQFGRASIESVD | |
| | | | TAYLTSLLEQGIVPIVAGFQGIDPNGDVTTLGRGGSDTTAVA | |
| | | | LAAALRADECQIFTDVSGVFTTDPNIDSSARRLDVIGFDVML | |
| | | | EMAKLGAKVLHPDSVEYAQRFKVPLRVLSSFEAGQGTLIQFG | |
| | | | DESELAMAASVQGIAINKALATLTIEGLFTSSERYQALLACL | |
| | | | ARLEVDVEFITPLKLNEISPVESVSFMLAEAKVDILLHELEV | |
| | | | LSESLDLGQLIVERQRAKVSLVGKGLQAKVGLLTKMLDVLGN | |
| | | | ETIHAKLLSTSESKLSTVIDERDLHKAVRALHHAFELNKV | |
| lysC | Corynebacterium | CAD89081 | MALVVQKYGGSSLESAERIRNVAERIVATKKAGNDVVVVCSA | 202 |
| | glutamicum | | MGDTTDELLELAAAVNPVPPAREMDMLLTAGERISNALVAMA | |
| | | | IESLGAEAQSFTGSQAGVLTTERHGNARIVDVTPGRVREALD | |
| | | | EGKICIVAGFQGVNKETRDVTTLGRGGSDTTAVALAAALNAD | |
| | | | VCEIYSDVDGVYTADPRIVPNAQKLEKLSFEEMLELAAVGSK | |
| | | | ILVLRSVEYAPAFNVPLRVRSSYSNDPGTLIAGSMEDIPVEE | |
| | | | AVLTGVATDKSEAKVTVLGISDKPGEAAKVFPALADAEINID | |
| | | | MVLQNVSSVEDGTTDITFTCPRSDGRRAMEILKKLQVQGNWT | |
| | | | NVLYDDQVGKVSLVGAGMKSHPGVTAEFMEALRDVNVNIELI | |
| | | | STSEIRISVLIREDDLDAAAPALHEQFQLGGEDEAV | |
| | | | VYAGTGR | |
| asparto | Escherichia | AAA24095 | MSEIVVSKFGGTSVADFDAMNRSADIVLSDANVRLVVLSASA | 203 |
| kinase | coli | | GITNLLVALAEGLEPGERFEKLDAIRNIQFAILERLRYPNVI | |
| | | | REEIERLLENITVLAEAAALATSPALTDELVSHGELMSTLLF | |
| | | | VEILRERDVQAQWFDVRKVMRTNDRFGRAEPDIAALAELAAL | |
| | | | QLLPRLNEGLVITQGFIGSENKGRTTTLGRGGSDYTAALLAE | |
| | | | ALHASRVDIWTDVPGIYTTDPRVVSAAKRIDEIAFAEAAEMA | |
| | | | TFGAKVLHPATLLPAVRSDIPVFVGSSKDPRAGGTLVCNKTE | |
| | | | NPPLFRALALRRNQTLLTLHSLNMLHSRGFLAEVFGILARHN | |
| | | | ISVDLITTSEVSVALTLDTTGSTSTGDTLLTQSLLMELSALC | |
| | | | RVEVEEGLALVALIGNDLSKACGVGKEVFGVLEPFNIRMICY | |
| | | | GASSHNLCFLVPGEDAEQVVQKLHSNLFE | |
| asd | Corynebacterium | CAA40504 | MTTIAVVGATGQVGQVMRTLLEERNFPADTVRFFASPRSAGR | 204 |
| | glutamicum | | KIEFRGTEIEVEDITQATEESLKDIDVALFSAGGTASKQYAP | |
| | | | LFAAAGATVVDNSSAWRKDDEVPLIVSEVNPSDKDSLVKGII | |
| | | | ANPNCTTMAANPVLKPLHDAAGLVKLHVSSYQAVSGSGLAGV | |
| | | | ETLAKQVAAVGDHNVEFXTHDGQAADAGDVGPYVSPIAYNVLP | |
| | | | FAGNLVDDGTFETDEEQKLRNESRKILGLPDLKVSGTCVRVP | |
| | | | VFTGHTLTIHAEFDKAITVDQAQEILGAASGVKLVDVPTPLA | |
| | | | AAGIDESLVGRIRQDSTVDDNRGLVLVVSGDNLRKGAALNTI | |
| | | | QIAELLVK | |
| asd | Escherichia | P00353 | MKNVGFIGWRGMVGSVLMQRMVEERDFDAIRPVFFSTSQLGQ | 205 |
| | coli | | AAPSFGGTTGTLQDAFDLEALKALDIIVTCQGGDYTNEIYPK | |
| | | | LRESGWQGYWIDAASSLRMKDDAIIILDPVNQDVITDGLNNG | |
| | | | IRTFVGGNCTVSLMLMSLGGLFANDLVDWVSVATYQAASGGG | |
| | | | ARHMRELLTQMGHLYGHVADELATPSSATLDIERKVTTLTRS | |
| | | | GELPVDNFGVPLAGSLIPWIDKQLDNGQSREEWKGQAETNKI | |
| | | | LNTSSVIPVDGLCVRVGALRCHSQAFTIKLKKDVSIPTVEEL | |
| | | | LAAHNPWAKVVPNDREITMRELTPAAVTGTLTTPVGRLRKLN | |
| | | | MGPEFLSAFTVGDQLLWGAAEPLRRMLRQLA | |
| ppc | Thermobifida | ZP_00058586 | MTRDSARQEMPDQLRRDVRLLGEMLGTVLAESGGQDLLDDVE | 7 |
| | fusca | | RLRRAVIGAREGTVEGKEITELVASWPLERAKQVARAFTVYF | |
| | | | HLVNLAEEHHRMRALRERDDAATPQRESLAAAVHSIREDAGP | |
| | | | ERLRELIAGMEFHPVLTAHPTEARRRAVSTAIQRISAQLERL | |
| | | | HAAHPGSGAEAEARRRLLEEIDLLWRTSQLRYTKMDPLDEVR | |
| | | | TAMAAFDETIFTVIPEVYRSLDPALDPEGCGRRPALAKAFVR | |
| | | | YGSWIGGDRDGNPFVTHEVTREAITIQSEHVLRALENACERI | |
| | | | GRTHTEYTGLTPPSAELRAALSSARAAYPRLMQEIIKRSPNE | |
| | | | PHRQLLLLAAERLRATRLRNADLGYPNPEAFLADLRTVQESL | |
| | | | AAAGAVRQAYGELQNLIWQAETFGFHLAELEIRQHSAVHAAA | |
| | | | LKEIRAGGELSERTEEVLATLRVVAWIQERFGVEACRRYIVS | |
| | | | FTQSADDIAAVYELAEHAMPPGKAPILDVIPLFETGADLDAA | |
| | | | PQVLDGMLRLPAVQRRLEQTGRRMEVMLGYSDSAKDVGPVSA | |
| | | | TLRLYDAQARLAEWAREHDIKLTLFHGRGGALGRGGGPANRA | |
| | | | VLAQAPGSVDGRFKVTEQGEVIFARYGQRAIAHRHIEQVGHA | |
| | | | VLMASTESVQRRAAEAAARFRGMADRIAEAAHAAYRALVDTE | |
| | | | GFAEWFSRVSPLEELSELRLGSRPARRSAARGLDDLRAIPWV | |
| | | | FAWTQTRVNLPGWYGLGSGLAAVDDLEALHTAYKEWPLFASL | |
| | | | LDNAEMSLAKTDRVIAERYLALGGRPELTEQVLAEYDRTREL | |
| | | | VLKVTRHTRLLENRRVLSRAVDLRNPYVDALSHLQLRALEAL | |
| | | | RTGEADRLSEEDRNHLERLLLLSVNGVAAGLQNTG | |
| ppc | Mycobacterium | CAC30086 | MVEFSDAILEPIGAVQRTRVGREATEPMRADIRLLGTILGDT | 8 |
| | leprae (can be | | LREQNGDEVFDLVERVRVESFRVRRSEIDRADMARMFSGLDI | |
| | used to clone | | HLAIPIIRAFSHFALLANVAEDIHRERRRHIHLDAGEPLRDS | |
| | M. smegmatis | | SLAATYAKLDLAKLDSATVADALTGAVVSPVITAHPTETRRR | |
| | gene) | | TVFVTQRRITELMRLHAEGHTETADGRSIERELRRQILTLWQ | |
| | | | TALIRLARLQISDEIDVGLRYYSAALFHVIPQVNSEVRNALR | |
| | | | ARWPDAELLSGPILQPGSWIGGDRDGNPNVTADVVRRATGSA | |
| | | | AYTVVAHYLAELTHLEQELSMSARLITVTPELATLAASCQDA | |
| | | | ACADEPYRRALRVIRGRLSSTAAHILDQQPPNQLGLGLPPYS | |
| | | | TPAELCADLDTIEASLCTHGAALLADDRLALLREGVGVFGFH | |
| | | | LCGLDMRQNSDVHEEVVAELLAWAGMHQDYSSLPEDQRVKLL | |
| | | | VAELGNRRPLVGDRAQLSDLARGELAVLAAAAHAVELYGSAA | |
| | | | VPNYIISMCQSVSDVLEVAILLKETGLLDASGSQPYCPVGIS | |
| | | | PLFETIDDLHNGAAILHAMLELPLYRTLVAARGNWQEVMLGY | |
| | | | SDSNKDGGYLAANWAVYRAELALVDVARKTGIRLRLFHGRGG | |
| | | | TVGRGGGPSYQAILAQPPGAVNGSLRLTEQGEVIAAKYAEPQ | |
| | | | IARRNLESLVAATLESTLLDVEGLGDAAESAYAILDEvAGLA | |
| | | | RRSYAELVNTPGFVDYFQASTPVSEIGSLNIGNRPTSRKPTT | |
| | | | SIADLRAIPWVLAWSQSRVMLPGWYGTGSAFQQWVAAGPESE | |
| | | | SQRVEMLHDLYQRWPFFRSVLSNMAQVLAKSDLGLAARYAEL | |
| | | | VVDEALRRRVFDKIADEHRRTIAIHKLITGHDDLLADNPALA | |
| | | | RSVFNRFPYLEPLNHLQVELLRRYRSGHDDEMVQRGILLTMN | |
| | | | GLASALRNSG | |
| ppc | Streptomyces | Q9RNU9 | MSSADDQTTTTTSSELRADIRRLGDLLGETLVRQEGPELLEL | 9 |
| | coelicolor | | VEKVRRLTREDGEAAAELLRGTELETAAKLVRAFSTYFHLAN | |
| | | | VTEQVHRGRELGAKRAAEGGLLARTADRLKDADPEHLRETVR | |
| | | | NLNVRPVFTAHPTEAARRSVLNKLRRIAALLDTPVNESDRRR | |
| | | | LDTRLAENIDLVWQTDELRVVRPEPADEARNAIYYLDELHLG | |
| | | | AVGDVLEDLTAELERAGVKLPDDTRPLTFGTWIGGDRDGNPN | |
| | | | VTPQVTWDVLILQHEHGINDALEMIDELRGFLSNSIRYAGAT | |
| | | | EELLASLQADLERLPEISPRYKRLNAEEPYRLKATCIRQKLE | |
| | | | NTKQRLAKGTPHEDGRDYLGTAQLIDDLRIVQTSLREHRGGL | |
| | | | FADGRLARTIRTLAAFGLQLATMDVREHADAHHHALGQLFDR | |
| | | | LGEESWRYADMPREYRTKLLAKELRSRRPLAPSPAPVDAPGE | |
| | | | KTLGVFQTVRRALEVFGPEVIESYIISMCQGADDVFAAAVLA | |
| | | | REAGLIDLHAGWAKIGIVPLLETTDELKAADTILEDLLADPS | |
| | | | YRRLVALRGDVQEVMLGYSDSSKFGGITTSQWEIHRAQRRLR | |
| | | | DVAHRYGVRLRLFHGRGGTVGRGGGPTHDAILAQPWGTLEGE | |
| | | | IKVTEQGEVISDKYLIPALARENLELTVAATLQASALHTAPR | |
| | | | QSDEALARWDAANDVVSDAAHTAYRHLVEDPDLPTYFLASTP | |
| | | | VDQLADLHLGSRPSRRPGSGVSLDGLRAIPWVFGWTQSRQIV | |
| | | | PGWYGVGSGLKALREAGLDTVLDEMHQQWHFFRNFISNVEMT | |
| | | | LAKTDLRIAQHYVDTLVPDELKHVFDTIKAEHELTVAEVLRV | |
| | | | TGESELLDADPVLKQTFTIRDAYLDPISYLQVALLGRQREAA | |
| | | | AANEDPDPLLARALLLTVNGVAAGLRNTG | |
| ppc | Erwinia | | MNEQYSAMRSNVSMLGKLLGDTIKDALGANILERVETIRKLS | 10 |
| | chrysanthemi | | KASPAGSETHRQELLTTLQNLSNDELLPVARAFSQFLNLTNT | |
| | | | AEQYNSISPHGEAASNPEALATVFRSLKSRDNLSDKDIRDAV | |
| | | | ESLSIELVLTAHPTEITRRTLIHKLVEVNTCLKQLDHDDLAD | |
| | | | YERHQIMRRLRQLIAQYWHTDEIRKIRPTPVDEAKWGFAVVE | |
| | | | NSLWEGVPAFLRELDEQMGKELGYRLFVDSVPVRFTSWMGGD | |
| | | | RDGNPNVTSEVTRRVLLLSRWKAADLFLRDVQVLVSELSMTT | |
| | | | CTPELQQLAGGDEVQEPYRELMKALRAQLTATLDYLDARLKD | |
| | | | EQRMPPKDLLVTNEQLWEPLYACYQSLHACGMGIIADGQLLD | |
| | | | TLRRVRCFGVPLVRIDVRQESTRHTDALAEITRYLGLGDYES | |
| | | | WSESDKQAFLIRELNSKRPLLPRQWEPSADTQEVLETCRVIA | |
| | | | ETPRDSIAAYVISMARTPSDVLAVHLLLKEAGCPYALPVAPL | |
| | | | FETLDDLNNADSVMIQLLNIDWYRGFIQGKQMVMIGYSDSAK | |
| | | | DAGVMAASWAQYRAQDALIKTCEKYGIALTLFHGRGGSIGRG | |
| | | | GAPAHAALLSQPPGSLKGGLRVTEQGEMIRFKFGLPEVTISS | |
| | | | LSLYTSAILEANLLPPPEPKQEWHHIMNELSRISCDMYRGYV | |
| | | | RENPDFVPYFRAATPELELGKLPLGSRPAKRRPNGGVESLRA | |
| | | | IPWIFAWTQNRLMLPAWLGAGAALQKVIDDGHQNQLEAMCRD | |
| | | | WPFFSTRIGMLEMVFAKAIJLWLAEYYDQRLVDEKLWSLGKQL | |
| | | | REQLERDIKAVLTISNDDHLMADLPWIAESIALRNVYTDPLN | |
| | | | VLQAELLHRSRQQETLDPQVEQALMVTIAGVAAGMRNTG | |
| ppc | Coryne- | P12880 | MTDFLRDDIRFLGQILGEVIAEQEGQEVYELVEQARLTSFDI | 206 |
| | bacterium | | AKGNAEMDSLVQVFDGITPAKATPIARAFSHFALLANLAEDL | |
| | glutamicum | | YDEELREQALDAGDTPPDSTLDATWLKLNEGNVGAEAVADVL | |
| | | | RNAEVAPVLTAHPTETRRRTVFDAQKWITTHMRERHALQSAE | |
| | | | PTARTQSKLDEIEKNIRRRITILWQTALIRVARPRIEDEIEV | |
| | | | GLRYYKLSLLEEIPRINRDVAVELRERFGEGVPLKPVVKPGS | |
| | | | WIGGDHDGNPYVTAETVEYSTHPAAETVLKYYARQLHSLEHE | |
| | | | LSLSDRMNKVTPQLLALADAGHNDVPSRVDEPYRRAVHGVRG | |
| | | | RILATTAELIGEDAVEGVWFKVFTPYASPEEFLNDALTIDHS | |
| | | | LRESKDVLIADDRLSVLISAlESFGFNLYALDLRQNSESYED | |
| | | | VLTELFERAQVTANYRELSEAEKLEVLLKELRSPRPLIPHGS | |
| | | | DEYSEVTDRELGIFRTASEAVKKFGPRMVPHCIISMASSVTD | |
| | | | VLEPMVLLKEFGLIAANGDNPRGTVDVIPLFETIEDLQAGAG | |
| | | | ILDELWKIDLYRNYLLQRDNVQEVMLGYSDSMWGGYFSANW | |
| | | | ALYDAELQLVELCRSAGVKLRLFHGRGGTVGRGGGPSYDAIL | |
| | | | AQPRGAVQGSVRITEQGEIISAKYGNPETARRNLEALVSATL | |
| | | | EASLLDVSELTDHQRAYDIMSEISELSLKKYASLVHEDQGFI | |
| | | | DYFTQSTPLQEIGSLNIGSRPSSRKQTSSVEDLRAIPWVLSW | |
| | | | SQSRVMLPGWFGVGTALEQWIGEGEQATQRIAELQTLNESWP | |
| | | | FFTSVLDNMAQVMSKAELRLAKLYADLIPDTEVAERVYSVIR | |
| | | | EEYFLTKKMFCVITGSDDLLDDNPLLARSVQRRYPYLLPLNV | |
| | | | IQVEMMRRYRKGDQSEQVSRNIQLTMNGLSTALRNSG | |
| ppc | Escherichia | P00864 | MNEQYSALRSNVSMLGKVLGETIKDALGEHILERVETIRKLS | 207 |
| | coli | | KSSRAGNDANRQELLTTLQNLSMDELLPVAPAFSQFLNLANT | |
| | | | AEQYHSISPKGEAASNPEVIARTLRKLK&QPELSEDTIKKAV | |
| | | | ESLSLELVLTAHPTEITRRTLIHKMVEVNACLKQLDNKDlAD | |
| | | | YEHNQLMRRLRQLIAQSWHTDEIRKLRPSPVDEAKWGFAVVE | |
| | | | NSLWQGVPNYLRELNEQLEENLGYKLPVEFVPVRFTSWMGGD | |
| | | | RDGNPNVTADITRHVLLLSRWKATDLFLKDIQVLVSELSMVE | |
| | | | ATPELLALVGEEGAAEPYRYLMKNLRSRLMATQAWLEARLKG | |
| | | | EELPKPEGLLTQNEELWEPLYACYQSLQACGMGIIANGDLLD | |
| | | | TLRRVKCFGVPLVRIDIRQESTRHTEALGELTRYLGIGDYES | |
| | | | WSEADKQAFLIRELNSKRPLLPRNWQPSAETREVLDTCQVIA | |
| | | | EAPOGSIAAYVISMAKTPSDVLAVHLLLKEAGIGFAMPVAPL | |
| | | | FETLDDLNNANDVMTQLLNIDWYRGLIQGKQMVMIGYSDSAK | |
| | | | DAGVMAASWAQYQAQDALIKTCEKAGIELTLFHGRGGSIGRG | |
| | | | GAPAHAALLSQPPGSLKGGLRVTEQGEMIRFKYGLPEITVSS | |
| | | | LSLYTGAILEANLLPPPEPKESWRRIMDELSVISCDVYRGYV | |
| | | | RENKDFVPYFRSATPEQELGKLPLGSRPAKRRPTGGVESLRA | |
| | | | IPWIFAWTQNRLMLPAWLGAGTALQKVVEDGKQSELEAMCRD | |
| | | | WPFFSTRLGMLEMVFAKADLWLAEYYDQRLVDKALWPLGKEL | |
| | | | RNLQEEDIKVVLAIANDSHLMADLPWIAESIQLRNIYTDPLN | |
| | | | VLQAELLHRSRQAEKEGQEPDPRVEQALMVTIAGIA | |
| | | | AGMRNTG | |
| pyc | Streptomyces | CAB59603 | MFRKVLVANRGEIAIRAFRAGYELGARTVAVFPHEDRNSLHR | 12 |
| | coelicolor | | LKADEAYEIGEQGHPVRAYLSVEEIVRAARRAGADAVYPGYG | |
| | | | FLSENPELARACEEAGITFVGPSARILELTGNKARAVAAARE | |
| | | | AGVPVLGSSAPSTDVDELVRAADDVGFPVFVKAVAGGGGRGM | |
| | | | RRVEEPAQLREAIEAASREAASAFGDSTVFLEKAVVEPRHIE | |
| | | | VQILADGEGDVIHLFERDCSVQRRHQKVIELAPAPNLDPALR | |
| | | | ERICADAVNFARQIGYRNAGTVEFLVDRDGNHVFIEMNPRIQ | |
| | | | VEHTVTEEVTDVDLVQSQLRIAAGQTLADLGLAQENITLRGA | |
| | | | ALQCRITTEDPANGFRPDTGQISAYRSPGGSGIRLDGGTTHA | |
| | | | GTEISAHFDSMLVKLSCRGRDFTTAVNRARPAVAEFRIRGVA | |
| | | | TNIPFLQAVLDDPDFQAGRVTTSFIEQRPHLLTARHSADRGT | |
| | | | KLLTYLADVTVNKPHGERPELVDPLTKLPTASAGEPPAGSRQ | |
| | | | LLAELGPEGFARRLRESSTIGVTDTTFRDAHQSLLATRVRTK | |
| | | | DMLAVAPVVARTLPQLLSLECWGGATYDVALRFLAEDPWERL | |
| | | | AALREAVPNLCLQMLLRGRNTVGYTPYPTEVTDAFVQEAAAT | |
| | | | GIDIFRIFDALNDVEQMRPAIEAVRQTGSAVAEVALCYTADL | |
| | | | SDPSERLYTLDYYLRLAEQIVNAGAHVLAVKDMAGLLRAPAA | |
| | | | ATLVSALRREFDLPVHLHTHDTTGGQLATYLAAIQAGADAVD | |
| | | | GAVASMAGTTSQPSLSAIVAATDHTERPTGLDLQAVGDLEPY | |
| | | | WESVRKVYAPFEAGLASPTGRVYHHEIPGGQLSNLRTQAVAL | |
| | | | GLGDRFEDIEAMYAAADRMLGRLVKVTPSSKVVGDLALHLVG | |
| | | | AGVSPADFEQDPDRFDIPDSVVGFLRGELGTPPGGWPEPFRS | |
| | | | KALRGRAEARPLAELSEDDRDGLGKDRRATLNRLLFPGPARE | |
| | | | FDTHRASYGDTSILDSKDFFYGLRPGKEYTVDLDPGVRLLIE | |
| | | | LQAVGDADERGMRTVMSSLNGQLRPIQVRDRSAATDVPVTEK | |
| | | | ADRANPGHVAAPFAGVVTLAVAEGDEVEAGATVATIEAMKME | |
| | | | ASITAPKSGTVTRLAINRIQQVEGGDLLVQLA | |
| pyc | Mycobacterium | AAG30411.1 | MISKVLVANRGEIAIRAFRAAYEMGIATVAVYPYEDRNSLHR | 13 |
| | smegmatis | | LKADESYQIGEVGHPVRAYLSVDEIIRVAKHSGADAVYPGYG | |
| | | | FLSENPDLAAKCAEAGITFVGPSAEVLQLTGNKAPAIAAARA | |
| | | | AGLPVLSSSEPSSSVDELMAAAADMEFPLFVKAVSGGGGRGM | |
| | | | RRVTDRESLAEAIEAASREAESAFGDASVYLEQAVLNPRHIE | |
| | | | VQILADGAGNVMHLFERDCSVQRRHQKVVELAPAPNLSDELR | |
| | | | QQICADAVAFARQIGYSCAGTVEFLLDERGHHVFIECNPRIQ | |
| | | | VEHTVTEEITDVDLVSSQLRIAAGETLADLGLSQDRLVVRGA | |
| | | | AMQCRITTEVPANGFRPDTGRITAYRSPGGAGIRLDGGTNLG | |
| | | | ARISAHFDSMLVKLTCRGRDFSAAASRARRALAEFRIRGVST | |
| | | | NIPFLQAVIDDPDFPAGRVTTSFIDDRPHLLTSRSPADRGTR | |
| | | | ILNYLADITVNKPHGERPSTVYPQDKLPPLDLQAPPPAGSKQ | |
| | | | RLVELGPEGFAGWLRESKAVGVTDTTFRDAHQSLLATRVRTT | |
| | | | GLLMVAPYVARSMPQLLSIECWGGATYDVALRFLKEDPWERL | |
| | | | AALRESVPNICLQMLLRGRNTVGYTPYPELVTSAFVEEAAAT | |
| | | | GIDIFRIFDALNNVESMRPAIDAVRETGSTIAEVAMCYTGDL | |
| | | | SDPAENLYTLDYYLKLAEQIVEAGAHVLAIKDMAGLLPAPAA | |
| | | | HTLVSALRSRFDLPVHVHTHDTPGGQLATYLAAWSAGADAVD | |
| | | | GASAPMAGTTSQPALSSIVAAAAHTQYDTGLDLRAVCDLEPY | |
| | | | WEAVRKVYAPFESGLPGPTGRVYTHEIPGGQLSNLRQQAIAL | |
| | | | GLGDRFEEIEANYAAADRVLGRLVKVTPSSKVVGDLALALVG | |
| | | | AGITAEEFAEDPAKYDIPDSVIGFLRGELGDPPGGWPEPLRT | |
| | | | KALQGRGPARPVEKLTADDEALLAQPGPKRQAALNRLLFPGP | |
| | | | TAEFEAHRETYGDTSSLSANQFFYGLRYGEEHRVQLERGVEL | |
| | | | LIGLEAISEADERGMRTVMCIINGQLRPVLVRDRSIASEVPA | |
| | | | AEKADRNNADHIAAPFAGVVTVGVAEGDSVDAGQTIATIEAM | |
| | | | KMEAAITAPKAGTVARVAVAATAQVEGGDLLVVVS | |
| pyc | Coryne- | CAA70739 | MSTHTSSTLPAFKKILVANRGEIAVRAFRAALETGAATVAIY | 208 |
| | bacterium | | PREDRGSFHRSFASEAVRIGTEGSPVKAYLDIDEIIGAAKKV | |
| | glutamicum | | KADAIYPGYGFLSENAQLARECAENGITFIGPTPEVLDLTGD | |
| | | | KSRAVTAAKKAGLPVLAESTPSKNIDEIVKSAEGQTYPIFVK | |
| | | | AVAGGGGRGMRFVASPDELRKLATEASREAEAAFGDGAVYVE | |
| | | | RAVINPQHIEVQILGDHTGEVVHLYERDCSLQRRHQKVVEIA | |
| | | | PAQHLDPELRDRICADAVKFCRSIGYQGAGTVEFLVDEKGNH | |
| | | | VFIEMNPRIQVEHTVTEEVTEVDLVKAQMRLAAGATLKELGL | |
| | | | TQDKIKTHGAALQCRITTEDPNNGFRPDTGTITAYRSPOGAG | |
| | | | VRLDGAAQLGGEITAHFDSMLVKMTCRGSDFETAVAPAQRAL | |
| | | | AEFTVSGVATNIGFLRALLREEDFTSKRIATGFIADHPHLLQ | |
| | | | APPADDEQGRILDYLADVTVNKPHGVRPKDVAAPIDKLPNIK | |
| | | | DLPLPRGSRDRLKQLGPAAFARDLREQDALAVTDTTFRDAHQ | |
| | | | SLLATRVRSFALKPAAEAVAKLTPELLSVEAWGGATYDVANR | |
| | | | FLFEDPWDRLDELREAMPNVNIQMLLRGRNTVGYTPYPDSVC | |
| | | | RAFVKEAASSGVDIFRIFDALNDVSQMRPAIDAVLETNTAVA | |
| | | | EVANAYSGDLSDPNEKLYTLDYYLKMAEEIVKSGAHILAIKD | |
| | | | MAGLLRPAAVTKLVTALRREFDLPVHVHTHDTAGGQLATYFA | |
| | | | AAQAGADAVDGASAPLSGTTSQPSLSAIVAAFAHTRRDTGLS | |
| | | | LEAVSDLEPYWEAVRGLYLPFESGTPGPTGRVYRHEIPGGQL | |
| | | | SNLRAQATALGLADRFELIEDNYAAVNEMLGRPTKVTPSSKV | |
| | | | VGDLALHLVGAGVDPADFAADPQKYDIPDSVIAFLRGELGNP | |
| | | | PGGWPEPLRTRALEGRSEGKAPLTEVPEEEQAHLDADDSKER | |
| | | | RNSLNRLLFPKPTEEFLEHRRRFGNTSALDDREFFYGLVEGR | |
| | | | ETLIRLPDVRTPLLVRLDAISEPDDKGMRNVVANVNGQIRPM | |
| | | | RVRDRSVESVTATAEKADSSNKGHVAAPFAGVVTVTVAEGDE | |
| | | | VKAGDAVAIIEAMKMEATITASVDGKIDRVVVPAATKVEGGD | |
| | | | LIVVVS | |
| dapA | Thermobifida | ZP_00058970 | MVGSTTPNAPFGQMLTANITPMLDNGEVDYDGVARLATYLVD | 14 |
| | fusca | | EQRNDGLIVNGTTGESATTSDEEKERILRTVIDAVGDRATIV | |
| | | | AGAGSNDTRHSIELARTAERAGADGLLLVTPYYNRPPQEGLL | |
| | | | RHFTAIADATGLPIMLYDIPGRTGTPIDSETLVRLAEHPRIV | |
| | | | ANKDAKDDLGASSWVMSRTDLAYYSGSDMLNLPLLSIGAAGF | |
| | | | VSVVGHVVGSELHDMIDAYRAGDVARALDIHRRLIPVYRGMF | |
| | | | RTQGVITTKAVLAMFGLPAGVVRAPLLDASPELKELLREDLA | |
| | | | MAGVKGPTGLASAHEDAASGREAERLTEGTA | |
| dapA | Mycobacterium | CAC30464 | MTTVGFDVPARLGTLLTANVTPFDADGSVDTAAATRLANRLV | 15 |
| | leprae (can be | | DAGCDGLVLSGTTGESPTTTDDEKLQLLRVVLEAVGDRARVI | |
| | used to clone | | AGAGSYDTAHSVRLVKACAGEGAHGLLVVTPYYSKPPQTGLF | |
| | M. smegmatis | | AHFTAVADATELPVLLYDTPGRSVVPIEPDTIRALASHPNIV | |
| | gene) | | GVKEAKADLYSGARIMADTGLAYYSGDDALNLPWLAVGAIGF | |
| | | | ISVISHLAAGQLRELLSAFGSGDITTARKINVAIGPLCSAMD | |
| | | | RLGGVTMSKAGLRLQGIDVGDPRLPQMPATAEQIDELAVDMR | |
| | | | AASVLR | |
| dapA | Mycobacterium | CAA15549 | MTTVGFDVAARLGTLLTAMVTPFSGDGSLDTATAARLANHLV | 16 |
| | tuberculosis | | DQGCDGLVVSGTTGESPTTTDGEKIELLRAVLEAVGDRARVI | |
| | (can be used to | | AGAGTYDTAHSIRLAKACAAEGAHGLLVVTPYYSKPPQRGLQ | |
| | clone M. | | AHFTAVADATELPMLLYDIPGRSAVPIEPDTIRALASHPNIV | |
| | smegmatis | | GVKDAKADLHSGAQIMADTGLAYYSGDDALNLPWLAMGATGF | |
| | gene) | | ISVIAHLAAGQLRELLSAFGSGDIATARKINIAVAPLCNAMS | |
| | | | RLGGVTLSKAGLRLQGIDVGDPRLPQVAATPEQIDALAADMR | |
| | | | AASVLR | |
| dapA | Streptomyces | CAA20295 | MAPTSTPQTPFGRVLTAMVTPFTADGALDLDGAQRLAAHLVD | 17 |
| | coelicolor | | AGNDGLIINGTTGESPTTSDAEKADLVRAVVEAVGDRAHVVA | |
| | | | GVGTNNTQHSIELARAAERVGAHGLLLVTPYYNKPPQEGLYL | |
| | | | HFTAIADAAGLPVMLYDIPGRSGVPINTETLVRLAEHPRIVA | |
| | | | NKDAKGDLGRASWAIARSGLAWYSGDDMLNLPLLAVGAVGFV | |
| | | | SVVGHVVTPELRAMVDAHVAGDVQKALEIHQKLLPVFTGMFR | |
| | | | TQGVMTTKGALALQGLPAGPLRAPMVGLTPEETEQLKIDLAA | |
| | | | GGVQL | |
| dapA | Erwinia | | MFTGSIVALVTPMDDKGAVDRASLKKLIDYHVASGTSAIVSV | 18 |
| | chrysanthemi | | GTTGESATLSHDEHGDVVMLTLELSDGRIPVIAGTGANSTAE | |
| | | | AISLTQRFNDTGVAGCLTVTPYYNKPTQNGLFLHFKAIAEHT | |
| | | | DLPQILYNVPSRTGCDMLPETVARLSEIKNIVAIKEATGNLS | |
| | | | RVSQIQELVHEDFILLSGDDASSLDFMQLGGDGVISVTANIA | |
| | | | AREMAALCELAAQGNFVEARRLNQRLMPLHQKLFVEPNPIPV | |
| | | | KWACKALGLMATDTLRLPMTPLTDAGRDVMEQAMKQAGLL | |
| dapA | Coryne- | C40626 | MSTGLTAKTGVEHFGTVGVAMVTPFTESGDIDIAAGREVAAY | 126 |
| | bacterium | | LVDKGLDSLVLAGTTGESPTTTAAEKLELLKAVREEVGDRAK | |
| | glutamicum | | LIAGVGTNNTRTSVELAEAAASAGADGLLVVTPYYSKPSQEG | |
| | | | LLAHFGAIAAATEVPICLYDIPGRSGIPIESDTMRRLSELPT | |
| | | | ILAVKDAKGDLVAATSLIKETGLAWYSGDDPLNLVWLALGGS | |
| | | | GFISVIGHAAPTALRELYTSFEEGDLVRAREINAKLSPLVAA | |
| | | | QGRLGGVSLAKAALRLQGINVGDPRLPIMAPNEQELEALRED | |
| | | | MKKAGVL | |
| dapA | Escherichia | NP_416973 | MFTGSIVAIVTPMDEKGNVCRASLKKLIDYHVASGTSAIVSV | 127 |
| | coli | | GTTGESATLNHDEHADVVMMTLDLADGR | |
| | | | IPVIAGTGANATAEAISLTQRFNDSGIVGCLTVTPYYNRPSQ | |
| | | | EGLYQHFKAIAEHTDLPQILYNVPSRTGCDLLPETVGRLAKV | |
| | | | KNIIGIKEATGNLTRVNQIKELVSDDFVLLSGDDASALDFMQ | |
| | | | LGGHGVISVTANVAARDMAQMCKLAAEGHFAEARVINQRLMP | |
| | | | LHNKLFVEPNPIPVKWACKELGLVATDTLRLPMTPITDSGRE | |
| | | | TVRAALKHAGLL | |
| hom | Streptomyces | CAC33918 | MRTRPLKVALLGCGVVGSKVARIMTTHAADLAARIGAPVELA | 19 |
| | coelicolor | | GVAVRRPDKVREGIDPALVTTDATALVKRGDIDVVVEVIGGI | |
| | | | EPARTLITTAFAHGASVVSANKALIAQDGAALHAAADEHGKD | |
| | | | LYYEAAVAGAIPLIRPLRESLAGDKVNRVLGIVNGTTNFILD | |
| | | | AMDSTGAGYQEALDEATALGYAEADPTADVEGFDAAAKAAIL | |
| | | | AGIAFHTRVRLDDVYREGMTEVTAADFASAKEMGCTIKLLAI | |
| | | | CERAADGGSVTARVHPAMIPLSHPLANVREAYNAVFVESDAA | |
| | | | GQLMFYGPGAGGSPTASAVLGDLVAVCRNRLGGATGPGESAY | |
| | | | AALPVSPMGDVVTRYHISLDVADKPGVLAQVATVFAEHGVSI | |
| | | | DTVRQSGKDGEASLVVVTHRASDAALGGTVEALRKLDTVRGV | |
| | | | ASIMRVEGE | |
| hom | Mycobacterium | AAD32592 | MSKKPIGVAVLGLGNVGSEVVRIIADSADDLAARIGAPLELR | 20 |
| | smegmatis | | GVGVRRVADDRGVPTELLTDDIDALVSRDDVDIVVEVMGPVE | |
| | | | PARKAILSALEQGKSVVTANKALMAMSTGELAQAAEKAHVDL | |
| | | | YFEAAVAGAIPVIRPLTQSLAGDTVRRVAGIVNGTTNYILSE | |
| | | | MDSTGADYTSALADASALGYAEADPTADVEGYDAAAKAAILA | |
| | | | SIAFHTRVTADDVYREGITTVSAEDFASAPALGCTIKLLAIC | |
| | | | ERLTSDEGKDRVSARVYPALVPLTHPLAAVNGAFNAVVVEAE | |
| | | | AAGRLMFYGQGAGGAPTAFAVMGDVVMAARNRVQGGRGPRES | |
| | | | KYAKLPIAPIGFIPTRYYVISIMNVADRPGVLSAVAAEF | |
| hom | Thermobifida | ZP_00058460 | MRRPEPAGAADRGRTRPRHRRTGGHHPLRGRHGQGRGGDPHL | 21 |
| | fusca | | CQCRRRYERQHPHPAVRCGVHLCAGLAAQRRRADAVPPGRQA | |
| | | | LRERRHRRARPLPPCRPASRRPGSSGRHRRLLLLHGQQLQPR | |
| | | | APACRGRGPREERPRPGATG~RRRPVAAGRRLSSGRRRSGHH | |
| | | | DEVLDTDNERRNGSHPLMALKVALLGCGVVGSQVVRLLNEQS | |
| | | | RELAERIGTPLEIGGIAVRRLDRARGTGVDPDLLTTDANGLV | |
| | | | TRDDIDLVVEVIGGIEPARSLILAAIQKGKSVVTANKALLAE | |
| | | | DGATTHAAAREAGVDVYYEASVAGAIPLLRPLRDSLAGDRVN | |
| | | | RVLGIVNGTTNYILDRMDSLGAGFTESLEEAQALGYAEADPT | |
| | | | ADVEGFDAAAKAAILARLAFHTPVTAADVHREGITEVSAADI | |
| | | | ASAKAMGCVVKLLAICQRSDDGSSIGVRVHPVMLPREHPLAS | |
| | | | VKGAYNAVFVEAESAGQLMFYGAGAGGVPTASAVLGDLVAVA | |
| | | | RNRLARTFVADGRADAKLPVHPMGETITSYHVALDVADRPGV | |
| | | | LAGVAKVFAANGVSIKHVRQEGRGDDAQLVLVSHTAPDAALA | |
| | | | RTVEQLRNHEDVRAVASVMRVETFDNER | |
| hom | Coryne- | CAA68614 | MTSASAPSFNPGKGPGSAVGIALLGFGTVGTEVMRLMTEYGD | 209 |
| | bacterium | | ELAHRIGGPLEVRGIAVSDISKPREGVAPELLTEDAFALIER | |
| | glutamicum | | EDVDIVVEVIGGIEYPREVVLAALKAGKSVVTANKALVAAHS | |
| | | | AELADAAEAANVDLYFEAAVAGAIPVVGPLRRSLAGDQIQSV | |
| | | | MGIVNGTTNFILDAMDSTGADYADSLAEATRLGYAEADPTAD | |
| | | | VEGHDAASKAAILASIAFHTRVTADDVYCEGISNISAADIEA | |
| | | | AQQAGHTIKLLAICEKFTNKEGKSAISARVHPTLLPVSHPLA | |
| | | | SVNKSFNAIFVEAEAAGRLMFYGNGAGGAPTASAVLGDVVGA | |
| | | | ARNKVHGGRAPGESTYANLPIADFGETTTRYHLDMDVEDRVG | |
| | | | VLAELASLFSEQGISLRTIRQEERDDDARLIVVTHSALESDL | |
| | | | SRTVELLKAKPVVKAINSVIRLERD | |
| metL | Escherichia | CAA23585 | SVIAQAGAKGRQLHKFGGSSLADVKCYLRVAGIMAEYSQPDD | 210 |
| (bifunctional; | coli | | MMVVSAAGSTTNRLISWLKLSQTDRLSAHQVQQTLRRYQCDL | |
| contains | | | ISGLLPAEEADSLISAFVSDLERLAALLDSGINDAVYAEVVG | |
| hom | | | HGEVWSARLMSAVLNQQGLPAAWLDAREFLRAERAAQPQVDE | |
| activity) | | | GLSYPLLQQLLVQHPGKRLVVTGFISRNNAGETVLLGRNGSD | |
| | | | YSATQIGALAGVSRVTIWSDVAGVYSADPRKVKDACLLPLLR | |
| | | | LDEASELARLAAPVLHARTLQPVSGSEIDLQLRCSYTPDQGS | |
| | | | TRIERVLASGTGARIVTSHDDVCLIEFQVPASQDFKLGHKEI | |
| | | | DQILKRAQVRPLAVGVHNDRQLLQFCYTSEVADSALKILDEA | |
| | | | GLPGELRLRQGLALVAMVGAGVTRNPLHCHRFWQQLKGQPVE | |
| | | | FTWQSDDGISLVAVLRTGPTESLIQGLHQSVFPAEKRIGLVL | |
| | | | FGKGNIGSRWLELFAREQSTLSARTGFEFVLAGVVDSRRSLL | |
| | | | SYDGLDASRALAFFNDEAVEQDEESLFLWMRAHPYDDLVVLD | |
| | | | VTASQQLADQYLDFASHGFHVISANKLAGASDSNKYRQIHDA | |
| | | | FEKTGRHWLYNATVGAGLPINHTVRDLIDSGDTILSISGIFS | |
| | | | GTLSWLFLQFDGSVPFTELVDQAWQQGLTEPDPRDDLSGKDV | |
| | | | SRKLVILAREAGYNIEPDQVRVESLVPAHCEGGSIDHFFENG | |
| | | | DELNEQMVQRLEAAREMGLVLRYVARFDANGKARVGVEAVRE | |
| | | | DHPLRSLLPCDNVFAIESRWYRDNPLVIRGPGAGRDVTAGAI | |
| | | | QSDINRLAQLL | |
| thrA | Escherichia | AAA97301 | MRVLKFGGTSVANAERFLRVADILESNARQGQVATVLSAPAK | 211 |
| (bifunctional; | coli | | ITNHLVAMIEKTISGQDALPNISDAERIFAELLTGLAAAQPG | |
| contain | | | FPLAQLKTFVDQEFAQIKHVLHGISLLGQCPDSINAALICRG | |
| hom | | | EKMSIATMAGVLEARGHNVTVIDPVEKLLAVGHYLESTVDIA | |
| activity | | | ESTRRIAASRIPADHMVLMAGFTAGNEKGELVVLGRNGSDYS | |
| | | | AAVLAACLRADCCETWTDVDGVYTCDPRQVPDARLLKSMSYQ | |
| | | | EAMELSYFGAKVLHPRTITPIAQFQIPCLIKNTGNPQAPGTL | |
| | | | IGASRDEDELPVKGISNLNNMAMFSVSGPGMKGMVGMAARVF | |
| | | | AANSRARISVVLITQSSSEYSISFCVPQSDCVRAERANQEEF | |
| | | | YLELKEGLLEPLAVTERLAIISVVGDGMRTLRGISAKFFAAL | |
| | | | ARANINIVAIAQGSSERSISVVVNNDDATTGVRVTHQMLFNT | |
| | | | DOVIEVFVIGVGGVGGALLEQLKRQQSWLKNKNIDLRVCGVA | |
| | | | NSKALLTNVHGLNLENWQEELAQAKEPFNLGRLIRLVKEYHL | |
| | | | LNPVIVDCTSSQAVADQYADFLREGFHVVTPNKKANTSSMDY | |
| | | | YHQLRYAAEKSRRKFLYDTNVGAGLPVIENLQNLLNAGDELM | |
| | | | KFSGILSGSLSYIFGKLDEGMSFSEATTLAREMGYTEPDPRD | |
| | | | DLSGMDVARKLLILARETGRELELADIEIEPVLPAEFNAEGD | |
| | | | VAAFMANLSQLDDLFAARVAKARDEGKVLRYVGNIDEDGVCR | |
| | | | VKIAEVDGNDPLFKVKNGENALAFYSHYYQPLPLVLRGYGAG | |
| | | | NDVTAAGVFADLLRTLSWKLGV | |
| metA | Mycobacterium | CAA17113 | MTISDVPTQTLPAEGEIGLIDVGSLQLESGAVIDDVCIAVQR | 22 |
| | tuberculosis | | WGKLSPARDNVVVVLHALTGDSHITGPAGPGHPTPGWWDGVA | |
| | (can be used to | | GPGAPIDTTRWCAVATNVLGGCRGSTGPSSLARDGKPWGSRF | |
| | clone M. | | PLISIRDQVQADVAALAALGITEVAAVVGGSMGGARALEWVV | |
| | smegmatis | | GYPDRVRAGLLLAVGARATADQIGTQTTQIAAIKADPDWQSG | |
| | gene) | | DYHETGPAPDAGLRLARRFAHLTYRGEIELDTRFANHNQGNE | |
| | | | DPTAGGRYAVQSYLEHQGDKLLSRFDAGSYVILTEALNSHDV | |
| | | | GRGRGGVSAALRACPVPVVVGGITSDRLYPLRLQQELADLLP | |
| | | | GCAGLRVVESVYGHDGFLVETEAVGELIRQTLGLAD | |
| | | | REGACRR | |
| metA | Mycobacterium | CAB10992 | MTISKVPTQKLPAEGEVGLVDIGSLTTESGAVIDDVCIAVQR | 23 |
| | leprae (can be | | WGELSPTRDNVVMVLHALTGDSHITGPAGPGHPTPGWWDWIA | |
| | used to clone | | GPGAPIDTNRWCAIATNVLGGCRGSTGPSSLARDGKPWGSRF | |
| | M. smegmatis | | PLISIRDQVEADIAALAANGITKVAAVVGGSMGGARALEWII | |
| | gene) | | GHPDQVPAGLLLAVGVRATADQIGTQTTQIAAIKTDPNWQGG | |
| | | | DYYETGRAPENGLTIARRFAHLTYRSEVELDTRFANNNQGNE | |
| | | | DPATGGRYAVQSYLEHQGDKLLARFDAGSYVVLTETLNSHDV | |
| | | | GRGRGGIGTALRGCPVPVVVGGITSDRLYPLRLQQELAEMLP | |
| | | | GCTGLQVVDSTYGHDGFLVESEAVGKLIRQTLELADVGSKED | |
| | | | ACSQ | |
| metA | Thermobifida | ZP_00058188 | MSHDTTPPLPATGAWREGDPPGDRRWVELSEPLPLETGGELP | 24 |
| | fusca | | GVRLAYETWGSLNEDRSNAVLVLHALTGDSHVVGPEGPGHPS | |
| | | | PGWWEGIIGPGLALDTDRYFVVAPNVLGGCQGSTGPSSTAPD | |
| | | | GRPWGSRFPRITIRDTVPAEFALLREFGIHSWAAVLGGSMGG | |
| | | | MRALEWAATYPERVRRLLLLASPAASSAQQIAWAAPQLHAIR | |
| | | | SDPYWHGGDYYDRPGPGPVTGMGIARRIAHITYRGATEFDER | |
| | | | FGRNPQDGEDPMAGGRFAVESYLDHHAVKLARRFDAGSYVVL | |
| | | | TQAMNTHDVGRGRGGVAQALRRVTARTMVAGVSSDFLYPLAQ | |
| | | | QQELADGIPGADEVRVIESASGHDGFLTEINQVSVLI | |
| | | | KELLAQ | |
| metA | Corynebacterium | AAC06035 | MPTLAPSGQLEIQAIGDVSTEAGAIITNAEIAYHRWGEYRVD | 212 |
| | glutamicum | | KEGRSNVLIEHALTGDSNAADWWAADLLGPGKAINTDIYCVI | |
| | | | CTNVIGGCNGSTGPGSMHPDGNFWGWRFPATSIRDQVNAEKQ | |
| | | | FLDALGITTVAAVVLLGGSMGGARTLEWAAMYPETVGAAAVL | |
| | | | AVSARASAWQIGIQSAQIKAIENDHHWHEGNYYESGCNPATG | |
| | | | LGAARRIAHLTYRGELEIDERFGTKAQKNENPLGPYRKPDQR | |
| | | | FAVESYLDYQADKLVQRFDAGSYVLLTDALNRHDIGRDRGGL | |
| | | | NKALESIKVPVLVAGVDTDILYPYHQQEHLSRNLGNLLAMAK | |
| | | | IVSPVGHDAFLTESRQMDRIVRNFFSLISPDEDNPSTYIEFY | |
| | | | I | |
| metA | Escherichia | NP_418437 | MPIRVPDELPAVNFLREENVFVMTTSRASGQEIRPLKVLILN | 213 |
| | coli | | LMPKKIETENQFLRLLSNSPLQVDIQLLRIDSRESRNTPAEH | |
| | | | LNNFYCNFEDIQDQNFDGLIVTGAPLGLVEFNDVAYWPQIKQ | |
| | | | VLEWSKDHVTSTLFVCWAVQAALNILYGIPKQTRTEKLSGVY | |
| | | | EHHILHPHALLTRGFDDSFLAFHSRYADFPAALIRDYTDLEI | |
| | | | LAETEEGDAYLFASKDKRIAFVTGHPEYDAQTLAQEFFRDVE | |
| | | | AGLDPDVPYNYFPHNDPQNTPRASWRSHGNLLFTNWLNYYVY | |
| | | | QITPYDLRHMNPTLD | |
| metA | T. fusca | n/a | MSHDTTPPLPATGAWREGDPPGDRRWVELSEPLPLETGGELP | 281 |
| F269A | | | GVRLAYETWGSLNEDRSNAVLVLHALTGDSHVVGPEGPGHPS | |
| | | | PGWWEGIIGPGLALDTDRYFVVAPNVLGGCQGSTGPSSTAPD | |
| | | | GRPWGSRFPRITIRDTVRAEFALLREFGIHSWAAVLGGSMGG | |
| | | | MRALEWAATYPERVRRLLLLASPAASSAQQIAWAAPQLHAIR | |
| | | | SDPYWHGGDYYDRPGPGPVTGMGIARRIAHITYRGATEFDER | |
| | | | FGRNPQDGEDPMAGGRAAVESYLDHHAVKLARRFDAGSYVVL | |
| | | | TQAMNTHDVGRGRGGVAQALRRVTARTMVAGVSSDFLYPLAQ | |
| | | | QQELADGIPGADEVRVIESASGHDGFLTEINQVSVLIKELLA | |
| | | | Q | |
| metY | T. fusca | n/a | MALRPDRSIMTAEDTTPESTAADKWSFETKQIHAGAAPDPAT | 282 |
| F379A | | | NARATPIYQTTSYVFRDTQHGADLFSLAEPGNIYTRIMNPTQ | |
| | | | DVLEKRVAALEGGVAAVAFASGSAAITAAVLNLAGAGDHIVS | |
| | | | SPSLYGGTYNLFRYTLPKLGIEVTFIKDQDDLDEWPAAARDN | |
| | | | TKLFFAETLPNPANNVLDVRAVADVAHEVGVPLMVDNTVPTP | |
| | | | YLQRPIDHGADIVVHSATKFLGGHGTTIAGIVVDAGTFDFGA | |
| | | | HGDRFPGFVEPDPSYHGLKYWEALGPGAYAAKLRVQLLRDTG | |
| | | | AAISPFNSFLILQGIETLSLRMERHVANAQALAEWLESRDEV | |
| | | | AKVYYPGLPSSPYYEAAKKYLPKGAGAIVSFELHGGIEAGHA | |
| | | | AVDGTELFSQLVNIGDVRSLIVHPASTTHSQLTPEEQLASGV | |
| | | | TPGLVRLSVGLEHVDDLRADLEAGLRAAKAYQ | |
| metY | C. glutamicum | N/a | MPKYDNSNADQWGFETRSIHAGQSVDAQTSARNLPIYQSTAF | 283 |
| G232A | | | VFDSAEHAKQRFALEDLGPVYSRLTNPTVEALENRIASLEGG | |
| | | | VHAVAFSSGQAATTNAILNLAGAGDHIVTSPRLYGGTETLFL | |
| | | | ITLNRLGIDVSFVENPDDPESWQAAVQPNTKAFFGETFANPQ | |
| | | | ADVLDIPAVAEVAHRNSVPLIIDNTIATAALVRPLELGADVV | |
| | | | VASLTKFYTGNGSGLGGVLIDAGKFDWTVEKDGKPVFPYFVT | |
| | | | PDAAYHGLKYADLGAPAFGLKVRVGLLRDTGSTLSAFNAWAA | |
| | | | VQGIDTLSLRLERHNENAIKVAEFLNNHEKVEKVNFAGLKDS | |
| | | | PWYATKEKLGLKYTGSVLTFEIKGGKDEAWAFIDALKLHSNL | |
| | | | ANIGDVRSLVVHPATTTHSQSDEAGLARAGVTQSTVRLSVGI | |
| | | | ETIDDIIADLEGGFAAI | |
| metY | T. fusca | n/a | MALRPDRSIMTAEDTTPESTAADKWSFETKQIHAGAAPDPAT | 284 |
| G240A | | | NARATPIYQTTSYVFRDTQHGADLFSLAEPGNIYTRIMNPTQ | |
| | | | DVLEKRVAALEGGVAAVAFASGSAAITAAVLNLAGAGDHIVS | |
| | | | SPSLYGGTYNLFRYTLPKLGIEVTFIKDQDDLDEWHAAARDN | |
| | | | TKLFFAETLPNPANNVLDVRAVADVAHEVGVPLMVDNTVPTP | |
| | | | YLQRPIDHGADIVVHSATKFLGGHGTTIAAIVVDAGTFDFGA | |
| | | | HGDRFPGFVEPDPSYHGLKYWEALGPGAYAAKLRVQLLRDTG | |
| | | | AAISPFNSFLILQGIETLSLRNERHVANAQALAEWLESRDEV | |
| | | | AKVYYPGLPSSPYYEAAKKYLPKGAGAIVSFELHGGIEAGRA | |
| | | | FVDGTELFSQLVNIGDVRSLIVHPASTTHSQLTPEEQLASGV | |
| | | | TPGLVRLSVGLEHVDDLRADLEAGLRAAKAYQ | |
| metA | T. fusca | n/a | MSHDTTPPLPATGAWREGDPPGDRRWVELSEPLPLETGGELP | 285 |
| G81A | | | GVRLAYETWGSLNEDRSNAVLVLHALTGDSHVVGPEGPAHPS | |
| | | | PGWWEGIIGPGLALDTDRYFVVAPNVLGGCQGSTGPSSTAPD | |
| | | | GRPWGSRFPRITIRDTVRAEFALLREFGIHSWAAVLGGSMGG | |
| | | | MRALEWAATYPERVRRLLLLASPAASSAQQIAWAAPQLHAIR | |
| | | | SDPYWHGGDYYDRPGPGPVTGMGIARRIAHITYRGATEFDER | |
| | | | FGRNPODGEDPMAGGRFAVESYLDHHAVKLARRFDAGSYVVL | |
| | | | TQAMNTHDVGRGRGGVAQALRRVTARTMVAGVSSDFLYPLAQ | |
| | | | QQELADGIPGADEVRVIESASGHDGFLTEINQVSVLIKELLA | |
| | | | Q | |
| metA | C. glutamicum | n/a | MPTLAPSGQLEIQAIGDVSTEAGAIITNAEIAYHRWGEYRVD | 286 |
| K233A | | | KEGRSNVVLIEHALTGDSNAADWWADLLGPGKAINTDIYCVI | |
| | | | CTNVIGGCNGSTGPGSMHPDGNFWGNRFPATSIRDQVNAEKQ | |
| | | | FLDALGITTVAAVLGGSMGGARTLEWAANYPETVGAAAVLAV | |
| | | | SARASAWQIGIQSAQIKAIENDHHWHEGNYYESGCNPATGLG | |
| | | | AARRIAHLTYRGELEIDERFGTAAQKNENPLGPYRKPDQRFA | |
| | | | VESYLDYQADKLVQRFDAGSYVLLTDALNRHDIGRDRGGLNK | |
| | | | ALESIKVPVLVAGVDTDILYPYHQQEHLSRNLGNLLAMAKIV | |
| | | | SPVGHDAFLTESRQMDRIVRNFFSLISPDEDNPSTYIEFYI | |
| metY | Thermobifide | ZP_00058187 | MALRPDRSIMTAEDTTPESTAADKWSFETKQIHAGAAPDPAT | 25 |
| | fusca | | NARATPIYQTTSYVFRDTQHGADLFSLAEPGNIYTRIMNPTQ | |
| | | | DVLEKRVAALEGGVAAVAFASGSAAITAAVLNLAGAGDHIVS | |
| | | | SPSLYGGTYNLFRYTLPKLGIEVTFIKDQDDLDEWRAAARDN | |
| | | | TKLFFAETLPNPANNVLDVPAVADVAHEVGVPLMVDNTVPTP | |
| | | | YLQRPIDHGADIVVHSATKFLGGHGTTIAGIVVDAGTFDFGA | |
| | | | HGDRFPGFVEPDPSYHGLKYWEALGPGAYAAKLRVQLLRDTG | |
| | | | AAISPFNSFLILQGIETLSLRMERHVANAQALAEWLESRDEV | |
| | | | AKVYYPGLPSSPYYEAAKKYLPKGAGAIVSFELHGGIEAGRA | |
| | | | FVDGTELFSQLVNIGDVRSLIVHPASTTHSQLTPEEQLASGV | |
| | | | TPGLVRLSVGLEHVDDLRADLEAGLRAAKAYQ | |
| metY | Mycobacterium | CAA17112 | MSADSNSTDADPTAHWSFETKQIHAGQHPDPTTNARALPIYA | 26 |
| | tuberculosis | | TTSYTFDDTAHAAALFGLEIPGNIYTRIGNPTTDVVEQRIAA | |
| | | | LEGGVAALFLSSGQAAETFAILNLAGAGDHIVSSPRLYGGTY | |
| | | | NLFHYSLAKLGIEVSFVDDPDDLDTWQAAVRPNTKAFFAETI | |
| | | | SNPQIDLLDTPAVSEVAHRNGVPLIVDNTIATPYLIQPLAQG | |
| | | | ADIVVHSATKYLGGHGAAIAGVIVDGGNFDWTQGRFPGFTTP | |
| | | | DPSYHGVVFAELGPPAFALKARVQLLRDYGSAASPFNAFLVA | |
| | | | QGLETLSLRIERHVANAQRVAEFLAARDDVLSVNYAGLPSSP | |
| | | | WHERAKRLAPKGTGAVLSFELAGGIEAGKAFVNALKLHSHVA | |
| | | | NIGDVRSLVIHPASTTHAQLSPAEQLATGVSPGLVRLAVGIE | |
| | | | GIDDILADLELGFAAARRFSADPQSVAAF | |
| metY | M. smegmatis | | MVDGFLRRPQGKRGSAGSGPRETGKPDGGQPCVVVREPFTPT | 287 |
| | | | RGVHLYVRTRVRLALGAGRPAAFTPHSPPSSRRRPSMTTPDP | |
| | | | TENWSFETKQIHAGQSPDSATHARALPIYQTTSYTFDDTSHA | |
| | | | AALFGLEVPGNIYTRIGNPTTDVVEQRIAALEGGVAALFLSS | |
| | | | GQAAETFAILNIAKAGDHIVSSPRLYGGTYNLLHYTLPKLGI | |
| | | | ETTFVENPDDLESWRAAVRPNTKAFFAETISNPQIDILDIPN | |
| | | | VAAIAHEAGVPLIVDNTIATPYLIQPIAHGADIVVHSATKYL | |
| | | | GGHGSAIAGVIVDGGTFDWTNGKFPGFTEPDPSYHGVVFAEL | |
| | | | GAPAYALKARVQLLRDLGSAAAPFNAFLIAQGLETLSLRVER | |
| | | | HVANAQKVAHFLENHPDVSSVNYAGLPSSPWYELGRKLAPKG | |
| | | | TGAVLAFELSGGLEAGKAFVNALTLHSHVANIGDVRSLVIHP | |
| | | | ASTTHQQLSPEEQLSTGVTPGLVRLAVGLEGIDDIIADLEQG | |
| | | | FAAARPFSGAAQTAQTV | |
| metY | Corynebacterium | AAG49653 | MPKYDNSNADQWGFETRSIHAGOSVDAQTSARNLPIYQSTAF | 214 |
| | glutamicum | | VFDSAEHAKQRFALEDLGPVYSRLTNPTVEALENRIASLEGG | |
| | | | VHAVAFSSGQAATTNAILNLAGAGDHIVTSPRLYGGTETLFL | |
| | | | ITLNRLGIDVSFVENPDDPESWQAAVQPNTKAFFGETFANPQ | |
| | | | ADVLDIPAVAEVAHRNSVPLIIDNTIATAALVRPLELGADVV | |
| | | | VASLTKFYTGNGSGLGGVLIDGGKFDWTVEKDGKPVFPYFVT | |
| | | | PDAAYHGLKYADLGAPAFGLKVRVGLLRDTGSTLSAFNAWAA | |
| | | | VQGIDTLSLRLERHNENAIKVAEFLNNHEKVEKVNFAGLKDS | |
| | | | PWYATKEKLGLKYTGSVLTFEIKGGKDEAWAFIDALKLHSNL | |
| | | | ANIGDVRSLVVHPATTTHSQSDEAGLARAGVTQSTVRLSVGI | |
| | | | ETIDDIIADLEGGFAAI | |
| MetY | C. glutamicum | N/a | MPKYDNSNADQWGFETRSIHAGQSVDAQTSARNLPIYQSTAF | 288 |
| D231A | | | VFDSAEHAKQRFALEDLGPVYSRLTNPTVEALENRIASLEGG | |
| | | | VHAVAFSSGQAATTNAILNLAGAGDHIVTSPRLYGGTETLFL | |
| | | | ITLNRLGIDVSFVENPDDPESWQAAVQPNTKAFFGETFANPQ | |
| | | | ADVLDIPAVAEVAHRNSVPLIIDNTIATAALVRPLELGADVV | |
| | | | VASLTKFYTGNGSGLGGVLIAGGKFDWTVEKDGKPVFPYFVT | |
| | | | PDAAYHGLKYADLGAPAFGLKVRVGLLRDTGSTLSAFNAWAA | |
| | | | VQGIDTLSLRLERHNENAIKVAEFLNNHEKVEKVNFAGLKDS | |
| | | | PWYATKEKLGLKYTGSVLTFEIKGGKDEAWAFIDALKLHSNL | |
| | | | ANIGDVRSLVVHPATTTHSQSDEAGLARAGVTQSTVRLSVGI | |
| | | | ETIDDIIADLEGGFAAI | |
| metY | T. fusca | n/a | MALRPDRSIMTAEDTTPESTAADKWSFETKQIHAGAAPDPAT | 289 |
| D244A | | | NARATPIYQTTSYVFRDTQHGADLFSLAEPGNIYTRINNPTQ | |
| | | | DVLEKRVAALEGGVAAVAFASGSAAITAAVLNLAGAGDHIVS | |
| | | | SPSLYGGTYNLFRYTLPKLGIEVTFIKDQDDLDEWRAAARDN | |
| | | | TKLFFAETLPNPANNVLDVRAVADVAHEVGVPLMVDNTVPTP | |
| | | | YLQRPIDHGADIVVHSATKFLGGHGTTIAGIVVAAGTFDFGA | |
| | | | HGDRFPGFVEPDPSYHGLKYWEALGPGAYAAKLRVQLLRDTG | |
| | | | AAISPFNSFLILQGIETLSLRMERHVANAQALAEWLESRDEV | |
| | | | AKVYYPGLPSSPYYEAAKKYLPKGAGAIVSFELEGGIEAGRA | |
| | | | FVDGTELFSQLVNIGDVRSLIVHPASTTHSQLTPEEQLASGV | |
| | | | TPGLVRLSVGLEHVDDLRADLEAGLRAAKAYQ | |
| MetA | T. fusca | n/a | MSHDTTPPLPATGAWREGDPPGDRRWVELSEPLPLETGGELP | 290 |
| D287A | | | GVRLAYETWGSLNEDRSNAVLVLHALTGDSHVVGPEGPGHPS | |
| | | | PGWWEGIIGPGLALDTDRYFVVAPNVLGGCQGSTGPSSTAPD | |
| | | | GRPWGSRFPRITIRDTVRAEFALLREFGIHSWAAVLGGSMGG | |
| | | | MRALEWAATYPERVRRLLLLASPAASSAQQIAWAAPQLHAIR | |
| | | | SDPYWHGGDYYDRPGPGPVTGMGIARRIAHITYRGATEFDER | |
| | | | FGRNPQDGEDPMAGGRFAVESYLDHHAVKLARRFAAGSYVVL | |
| | | | TQANNTHDVGRGRGGVAQALRRVTARTMVAGVSSDFLYPLAQ | |
| | | | QQELADGIPGADEVRVIESASGHDGFLTEINQVSVLIKELLA | |
| | | | Q | |
| metY | T. fusca | n/a | MALRPDRSIMTAEDTTPESTAADKWSFETKQIHAGAAPDPAT | 291 |
| D394A | | NARATPIYQTTSYVFRDTQHGADLFSLAEPGNIYTRIMNPTQ | | |
| | | | DVLEKRVAALEGGVAAVAFASGSAAITAAVLNLAGAGDHIVS | |
| | | | SPSLYGGTYNLFRYTLPKLGIEVTFIKDQDDLDEWRAAARDN | |
| | | | TKLFFAETLPNPANNVLDVRAVADVAHEVGVPLMVDNTVPTP | |
| | | | YLQRPIDHGADIVVHSATKFLGGHGTTIAGIVVDAGTFDFGA | |
| | | | HGDRFPGFVEPDPSYHGLKYWEALGPGAYAAKLRVQLLRDTG | |
| | | | AAISPFNSFLILQGIETLSLRMERHVANAQALAEWLESRDEV | |
| | | | AKVYYPGLPSSPYYEAAKKYLPKGAGAIVSFELHGGIEAGRA | |
| | | | FVDGTELFSQLVNIGAVRSLIVHPASTTHSQLTPEEQLASGV | |
| | | | TPGLVRLSVGLEHVDDLRADLEAGLRAAKAYQ | |
| metK | Mycobacterium | CAB02194 | MSEKGRLFTSESVTEGHPDKICDAISDSVLDALLAADPRSRV | 27 |
| | tuberculosis | | AVETLVTTGQVHVVGEVTTSAKEAFADITNTVRARILEIGYD | |
| | (can be used to | | SSDKGFDGATCGVNIGIGAQSPDIAQGVDTAHEARVEGAADP | |
| | clone M. | | LDSQGAGDQGLMFGYAINATPELMPLPIALAHRLSRRLTEVR | |
| | smegmatis | | KNGVLPYLRPDGKTQVTIAYEDNVPVRLDTVVISTQHAADID | |
| | gene) | | LEKTLDPDIREKVLMTVLDDLAHETLDASTVRVLVNPTGKFV | |
| | | | LGGPMGDAGLTGRKIIVDTYGGWARHGGGAFSGKDPSKVDRS | |
| | | | AAYAMRWVAKNVVAAGLAERVEVQVAYAIGKAAPVGLFVETF | |
| | | | GTETEDPVKIEKAIGEVFDLRPGAIIRDLNLLRPIYAPTAAY | |
| | | | GHFGRTDVELPWEQLDKVDDLKRAI | |
| metK | Mycobacterium | CAC30052 | MSEKGRLFTSESVTEGHPDKICDAISDSILDALLAEDPCSRV | 28 |
| | leprae (can be | | AVETLVTTGQVHVVGEVTTLAKTAFADISNTVRERILDIGYD | |
| | used to clone | | SSDKGFDGASCGVNIGIGAQSSDIAQGVNTAHEVRVEGAADP | |
| | M. smegmatis | | LDAQGAGDQGLMFGYAINDTPELMPLPIALAHRLARRLTEVR | |
| | gene) | | KNGVLPYLRSDGKTQVTIAYEDNVPVRLDTVVISTQHAAGVD | |
| | | | LDATLAPDIREKVLNTVIDDLSHDTLDVSSVRVLVNPTGKFV | |
| | | | LGGPMGDAGLTGRKIIVDTYGGWARHGGGAFSGKDPSKVDRS | |
| | | | AAYAMRWVAKNIVAAGLAERIEVQVAYAIGKAAPVGLFVETF | |
| | | | GTEAVDPAKIEKAIGEVFDLRPGAIIRDLHLLRPIYAQTAAY | |
| | | | GHFGRTDVELPWEQLNKVDDLKRAI | |
| metK | Thermobifida | ZP_00057715 | MSRRLFTSESVTEGHPDKIADQISDAILDSMLRDDPHSRVAV | 29 |
| | fusca | | ETLITTGLVHVAGEVTTSTYVDIPTIIREKILEIGYDSSAKG | |
| | | | FDGASCGVSVSIGGQSPDIAQGVDNAYEAREEEIFDDLDRQG | |
| | | | AGDQGLMFGYAPELMPLPITLAHALSQRLAEVRRDGTIPYLR | |
| | | | PDGKTQVTVEYDGNRNNETPVRLDTVVVSSQHAPDIDLRELL | |
| | | | TPDIKEHVVDPVVARYNLEADNYRLLVNPTGRFEIGGPMGDA | |
| | | | GLTGRKIIVDTYGGYARHGGGAFSGKDPSKVDRSAAYATRWV | |
| | | | AKNIVAAGLADRVEVQVAYAIGKAHPVGVFLETFGTEKVAPE | |
| | | | QLEKAVLEVFDLRPAAIIRDLDLLRPIYSQTSVYGHFGRELP | |
| | | | DFTWERTDRVDALKAAVGA | |
| metK | Streptomyces | CAB76898 | MSRRLFTSESVTEGHPDKIADQISDTILDALLREDPTSRVAV | 30 |
| | coelicolor | | ETLITTGLVHVAGEVTTKAYADIANLVRGKILEIGYDSSKKG | |
| | | | FDGASCGVSVSIGAQSPDIAQGVDTAYENRVEGDEDELDRQG | |
| | | | AGDQGLMFGYASDETPTLMPLPVFLAHRLSKRLSEVRKNGTI | |
| | | | PYLRPDGKTQVTIEYDGDKAVRLDTVVVSSQHASDIDLESLL | |
| | | | APDIKEFVVEPELKALLEDGIKIDTENYRLLVNPTGRFEIGG | |
| | | | PMGDAGLTGRKIIIDTYGGMARHGGGAFSGKDPSKVDRSAAY | |
| | | | ANRWVAKNVVAAGLAARCEVQVAYAIGKAEPVGLFVETFGTA | |
| | | | KVDTEKIEKAIDEVFDLRPAAIIRALDLLRPIYAQTAAYGHF | |
| | | | GRELPDFTWERTDRVDALREAAGL | |
| metK | Coryne- | BAB98996 | MAQPTAVRLFTSESVTEGHPDKICDAISDTILDALLEKDPQS | 215 |
| | bacterium | | RVAVETVVTTGIVHVVGEVRTSAYVEIPQLVRNKLIEIGFNS | |
| | glutamicum | | SEVGFDGRTCGVSVSIGEQSQEIADGVDNSDEARTNGDVEED | |
| | | | DRAGAGDQGLMFGYATNETEEYMPLPIALAHRLSRRLTQVRK | |
| | | | EGIVPHLRPDGKTQVTFAYDAQDRPSHLDTVVISTQHDPEVD | |
| | | | RAWLETQLREHVIDWVIKDAGIEDLATGEITVLINPSGSFIL | |
| | | | GGPMGDAGLTGRKIIVDTYGGMARHGGGAFSGKDPSKVDRSA | |
| | | | AYAMRWVAKNIVAAGLADRAEVQVAYAIGRAKPVGLYVETFD | |
| | | | TNKEGLSDEQIQAAVLEVFDLRPAAIIRELDLLRPIYADTAA | |
| | | | YGHFGRTDLDLPWEAIDRVDELPAALKLA | |
| metK | Escherichia | AAA69109 | MAKNLFTSESVSEGHPDKIADQISDAVLDAILEQDPKARVAC | 216 |
| | coli | | ETYVKTGMVLVGGEITTSAWVDIEEITRNTVREIGYVHSDMG | |
| | | | FDANSCAVLSAIGKQSPDINQGVDRADPLEQGAGDQGLMFGY | |
| | | | ATNETDVLMPAPITYAHRLVQRQAEVRKNGTLPWLRPDAKSQ | |
| | | | VTFQYDDGKIVGIDAVVLSTQHSEEIDQKSLQEAVMEEIIKP | |
| | | | ILPAEWLTSATKFFINPTGRFVIGGPMGDCGLTGRKIIVDTY | |
| | | | GGMARHGGGAFSGKDPSKVDRSAAYAARYVAKNIVAAGLADR | |
| | | | CEIQVSYAIGVAEPTSIMVETFGTEKVPSEQLTLLVREFFDL | |
| | | | RPYGLIQMLDLLHPIYKETAAYGHFGREHFPWEKTDKAQLLR | |
| | | | DAAGLK | |
| metC | Mycobacterium | CAA16256 | MQDSIFNLLTEEQLRGRNTLKWNYFGPDVVPLWLAEMDFPTA | 59 |
| | tuberculosis | | PAVLDGVPACVDNEEFGYPPLGEDSLPRATADWCRQRYGWCP | |
| | this to clone | | RPDWVRVVPDVLKGMEVVVEFLTRPESPVALPVPAYMPFFDV | |
| | M. smegmatis | | LHVTGRQRVEVPMVQQDSGRYLLDLDALQAAFVRGAGSVIIC | |
| | gene) | | NPNNPLGTAFTEAELPAIVDIAARHGARVIADEIWAPVVYGS | |
| | | | RHVAAASVSEAAAEVVVTLVSASKGWNLPGLMCAQVILSNRR | |
| | | | DAHDWDRINMLHRMGASTVGIRAMIAAYHHGESWLDELLPYL | |
| | | | RANRDHLARALPELAPGVEVNAPDGTYLSWVDFRALALPSEP | |
| | | | AEYLLSKAKVALSPGIPFGAAVGSGFARLNFATTRAILDRAI | |
| | | | EAIAAALRDIID | |
| metC | Bifidobacterium | P_00121229 | MSMNNIPQSTTVSNATADVSCFDANHIDVTTIEDLKQVGSDK | 60 |
| | longum | | WTRYPGCIGAFIAEMDYGLAPCVAEAIEEATERGALGYIPDP | |
| | | | WKKEVARSCAAWQRRYGWDVDPTCIRPVPDVLEAFEVFLREI | |
| | | | VRAGNSIVVPTPAYMPFLSVPRLYGVEVLEIPMLCAGASESS | |
| | | | GRNDEWLFDFDAIEQAFANGCHAFVLCNPHNPIGKVLTREEM | |
| | | | LRLSDLAAKYNVRIFSDEIHAPFVYQGHTHVPFASINRQTAM | |
| | | | QAFTSTSASKSFNIPGTKCAQVILTNPDDLELWMRNAEWSEH | |
| | | | QTATIGAIATTAAYDGGAAWFEGVMAYIERNIALVNEQMRTR | |
| | | | FAKVRYVEPQGTYIAWLDFSPLGIGDPANYFFKKANVALTDG | |
| | | | RECGEVGRGCVRMNFAMPYPLLEECFDRMAAALEADGLL | |
| metC | Lactobacillus | CAD65601 | MQYDFNKVINRRGTYSTQWDYIQDRFGRSDILPFSISDTDFP | 61 |
| | plantarum | | VPVGVQEALEQRIKHPIYGYTRWNNEDYKNSIINWFSSQNQV | |
| | | | TINPDWILYSPSVVFSIATFIRMKSAVGESVAVFTPMYDAFY | |
| | | | HVIEDNQRVLAPVRLGSAQQDYSIDWDTLKAVLKQTATKILL | |
| | | | LTNPHNPTGKVFSDDELKHIVALCQQyNVFIISDDIHKDIVY | |
| | | | QKAAYTPVTEFTTKNVVLCCSATKTFNTPGLIGAYLFEPEAE | |
| | | | LREMFLCELKQKNALSSASILGIESQMAAYNTGSDYLVQLIT | |
| | | | YLQNNFDYLSTFLKSQLPEIRFKQPEATYLAWMDVSQLGLTA | |
| | | | EKLQDKLVNTGRVGIMSGTTYGDSHYLRMNIACPISKLQEGL | |
| | | | KRMEYGIRS | |
| metC | Coryne- | AAK69425 | MRFPELEELKNRRTLKWTRFPEDVLPLWVAESDFGTCPQLKE | 217 |
| | bacterium | | AMADAVEREVFGYPPDATGLNDALTGFYERRYGFGPNPESVF | |
| | glutamicum | | AIPDVVRGLKLAIEHFTKPGSAIIVPLPAYPPFIELPKVTGR | |
| | | | QAIYIDAHEYDLKEIEKAFADGAGSLLFCNPHNPLGTVFSEE | |
| | | | YIRELTDIAAKYDARIIVDEIHAPLVYEGTHVVAAGVSENAA | |
| | | | NTCITITATSKAWNTAGLKCAQIFFSNEADVKAWKNLSDITR | |
| | | | DGVSILGLIAAETVYNEGEEFLDESIQILKDNRDFAAAELEK | |
| | | | LGVKVYAPDSTYLMWLDFAGTKIEEAPSKILREEGKVMLNDG | |
| | | | AAFGGFTTCARLNFACSRETLEEGLRRIASVL | |
| metC | Escherichia | P06721 | MADKKLDTQLVNAGRSKKYTLGAVNSVIQRASSLVFDSVEAK | 218 |
| | coli | | KHATRNRANGELFYGRRGTLTHFSLQQANCELEGGAGCVLFP | |
| | | | CGAAAVANSILAFIEQGDHVLMTNTAYEPSQDFCSKILSKLG | |
| | | | VTTSWFDPLIGADIVKHLQPNTKIVFLESPGSITMEVHDVPA | |
| | | | IVAAVRSVVPDAIIMIDNTWAAGVLFKALDFGIDVSIQAATK | |
| | | | YLVGHSDAMIGTAVCNARCWEQLRENAYLMGQMVDADTAYIT | |
| | | | SRGLRTLGVRLRQHHESSLKVAEWLAEHPQVARVNHPALPGS | |
| | | | KGHEFWKRDFTGSSGLFSFVLKKKLNNEELANYLDNFSLFSM | |
| | | | AYSWGGYESLILANQPEHIAAIRPQGEIDFSGTLIRLHIGLE | |
| | | | DVDDLIADLDAGFARIV | |
| pck | C. glutamicum | | MTTAAIRGLQGEAPTKNKELLNWIADAVELFQPEAVVFVDGS | 292 |
| | | | QAEWDRMAEDLVEAGTLIKLNEEKRPNSYLARSNPSDVARVE | |
| | | | SRTFICSEKEEDAGPTNNWAPPQAMKDEMSKHYAGSMKGRTM | |
| | | | YVVPFCMGPISDPDPKLGVQLTDSEYVVMSMRIMTRMGIEAL | |
| | | | DKIGANGSFVRCLHSVGAPLEPGQEDVAWPCNDTKYITQFPE | |
| | | | TKEIWSYGSGYGGNAILAKKCYALRIASVMAREEGWMAEHML | |
| | | | ILKLINPEGKAYHIAAAFPSACGKTNLAMITPTIPGWTAQVV | |
| | | | GDDIAWLKLREDGLYAVNPENGFFGVAPGTNYASNPIANKTM | |
| | | | EPGNTLFTNVALTDDGDIWWEGMDGDAPAHLIDWMGNDWTPE | |
| | | | SDENAAHPNSRYCVAIDQSPAAAPEFNDWEGVKIDAILFGGR | |
| | | | RADTVPLVTQTYDWEHGTMVGALLASGQTAASAEAKVGTLRH | |
| | | | DPMAMLPFIGYNAGEYLQNWIDMGNKGGDKMPSIFLVNWFRR | |
| | | | GEDGRFLWPGFGDNSRVLKWVIDRIEGHVGADETVVGHTAKA | |
| | | | EDLDLDGLDTPIEDVKEALTAPAEQWANDVEDNAEYLTFLGP | |
| | | | RVPAEVHSQFDALKARISAAHA | |
| pck | E. coli | | MRVNNGLTPQELEAYGISDVHDIVYNPSYDLLYQEELDPSLT | 293 |
| | | | GYERGVLTNLGAVAVDTGIFTGRSPKDKYIVRDDTTRDTFWW | |
| | | | ADKGKGKNDNKPLSPETWQHLKGLVTRQLSGKRLFVVDAFCG | |
| | | | ANPDTRLSVRFITEVAWQAHFVKNMFIRPSDEELAGFKPDFI | |
| | | | VMNGAKCTNPQWKEQGLNSENFVAFNLTERMQLIGGTWYGGE | |
| | | | MKKGMFSMMNYLLPLKGIASMHCSANVGEKGDVAVFFGLSGT | |
| | | | GKTTLSTDPKRRLIGDDEHGWDDDGVFNFEGGCYAKTIKLSK | |
| | | | EAEPEIYNAIRRDALLENVTVREDGTIDFDDGSKTENTRVSY | |
| | | | PIYHIDNIVKPVSKAGHATKVIFLTADAFGVLPPVSRLTADQ | |
| | | | TQYHFLSGFTAKLAGTERGITEPTPTFSACFGAAFLSLHPTQ | |
| | | | YAEVLVKRMQAAGAQAYLVNTGWNGTGKRISIKDTPAIIDAI | |
| | | | LNGSLDNAETFTLPMFNLAIPTELPGVDTKILDPRNTYASPE | |
| | | | QWQEKAETLAKLFIDNFDKYTDTPAGAALVAAGPKL | |
| gdh | Strepto- | CAB82051 | MPAVPERAPVTTRSETQSTLDHLLTEIELRNPAQPEFHQAAH | 62 |
| | mycescoelicolor | | EVLETLAPVVAARPEYAEPGLIERLVEPERQVMFRVPWQDDQ | |
| | | | GRVRVNRGFRVEFNSALGPYKGGLRFHPSVNLGVIKFLGFEQ | |
| | | | IFKNALTGLGIGGGKGGSDFDPHGRSDAEVMRFCQSFMTELY | |
| | | | RHIGEHTDVPAGDIGVGGREIGYLFGQYRRITNRWESGVLTG | |
| | | | KGQGWGGSLIRPEATGYGNVLFAAAMLRERGEDLEGQTAVVS | |
| | | | GSGNVAIYTIEKLTALGANAVTCSDSSGYVVDEKGIDLDLLK | |
| | | | QIKEVERGRVDAYAERRGASARFVPGGSVWDVPADLALPSAT | |
| | | | QNELDENAAATLVRNGVKAVSEGAMMPTTPEAVHLLQKAGVA | |
| | | | FGPGKAANAGGVAVSALEMAQNHARTSWTAARVEEELADIMT | |
| | | | SIHTTCHETAERYDAPGDYVTGANIAGFERVADAMLAQGVI | |
| gdh | Thermobifida | ZP_00057948 | MRPEPEATMSANLDEKLSPIYEEILRRNPGEVEFHQAVREVL | 63 |
| | fusca | | ECLGPVVAKNPDISHAKTIERLCEPERQLIFRVPWMDDSGEI | |
| | | | HVNRGFRVEFSSSLGPYKGGLRFHPSVNLSIIKFLGFEQIFK | |
| | | | NSLTGLPIGGAKGGSDFDPKGRSDAEIMRFCQSFMTELYRHL | |
| | | | GEHTDVPAGDIGVGQREIGYLFGQYKRITNRYESGVFTGKGL | |
| | | | SWGGSQVRREATGYGCVLFTAEMLRARGDSLEGKRVSVSGSG | |
| | | | NVAIYAIEKAQQLGAHVVTCSDSNGYVVDEKGIDLELLKQVK | |
| | | | EVERGRVSDYAKRRGSHVRYIDSSSSSVWEVPCDIALPCATQ | |
| | | | NELTGRDAITLVRNGVGAVAEGANMPTTPEGIRVFAEAGVAF | |
| | | | APGKAANAGGVATSALEMQQNASRDSWSFEYTEKRLAEIMRH | |
| | | | IHDTCYETAERYGRPGDYVAGANIAAFEIVAEANLAQGLI | |
| gdh | Lactobacilus | CAD63684 | MSQATDYVQHVYQVIEHRDPNQTEFLEAINDVFKTITPVLEQ | 64 |
| | plantarum | | HPEYIEANILERLTEPERIIQFRVPWLDDAGHARVNRGFRVQ | |
| | | | FNSAIGPYKGGLRLHPSVNLSIVKFLGFEQIFKNALTGLPIG | |
| | | | GGKGGSDFDPKGKSDNEIMRFCQSFMTELSKYIGLDTDVPAG | |
| | | | DIGVGGREIGFLYGQYKRLRGADRGVLTGKGLNYGGSLARTE | |
| | | | ATGYGLAYYTNEMLKANQLSFPGQRVAISGAGNVAIYAIQKV | |
| | | | EELGGKVITCSDSNGYVIDENGIDFKIVKQIKEVERGRIKDY | |
| | | | ADRVASASYYEGSVWDAQVAYDIALPCATQNEISGDQAKNLI | |
| | | | ANGAKVVAEGANMPSSPEAIATYQAASLLYGPAKAANAGGVA | |
| | | | VSALEMSQNSMRLSWTFEEVDNRLKQIMQDIFAHSVAAADEY | |
| | | | HVSGDYLSGANIAGFTKVADAMLAQGLV | |
| gdh | Coryne- | CAA42048 | MTVDEQVSNYYDMLLKRNAGEPEFHQAVAEVLESLKLVLEKD | 219 |
| | bacterium | | PHYADYGLIQRLCEPERQLIFRVPWVDDQGQVHVNRGFRVQF | |
| | glutamicum | | NSALGPYKGGLRFHPSVNLGIVKFLGFEQIFKNSLTGLPIGG | |
| | | | GKGGSDFDPKGKSDLEIMRFCQSFMTELHRHIGEYRDVPAGD | |
| | | | IGVGGREIGYLFGHYRRMANQHESGVLTGKGLTWGGSLVRTE | |
| | | | ATGYGCVYFVSEMIKAKGESISGQKIIVSGSGNVATYAIEKA | |
| | | | QELGATVIGFSDSSGWVHTPNGVDVAKLREIKEVRRARVSVY | |
| | | | ADEVEGATYHTDGSIWDLKCDIALPCATQNELNGENAKTLAD | |
| | | | NGCRFVAEGANMPSTPEAVEVFRERDIRFGPGKATPEAVEVF | |
| | | | RERDIRFGPGKAVNVGGVATSALEMQQNASRETCAETAAEYG | |
| | | | HENDYVVGANIAGFKKVADAMLAQGVI | |
| gdh | Escherichia | BAA15550 | MDQTYSLESFLNHVQKRDPNQTEFAQAVREVMTTLWPFLEQN | 220 |
| | coli | | PKYRQMSLLERLVEPERVIQFRVVWVDDRNQIQVNRAWRVQF | |
| | | | SSAIGPYKGGMRFHPSVNLSILKFLGFEQTFKNALTTLPMGG | |
| | | | GKGGSDFDPKGKSEGEVMRFCQALMTELYRHLGADTDVPAGD | |
| | | | IGVGGREVGFMAGMMKKLSNNTACVFTGKGLSFGGSLIRPEA | |
| | | | TGYGLVYFTEAMLKRHGMGFEGMRVSVSGSGNVAQYAIEKAN | |
| | | | EFGARVITASDSSGTVVDESGFTKEKLARLIEIKASRDGRVA | |
| | | | DYAKEFGLVYLEGQQPWSLPVDIALPCATQNELDVDAAHQLI | |
| | | | ANGVKAVAEGANMPTTIEATELFQQAGVLFAPGKAANAGGVA | |
| | | | TSGLEMAQNAARLGWKAEKVDARLHHIMLDIHHACVEHGGEG | |
| | | | EQTNYVQGANIAGFVKVADANLAQGVI | |
| ddh | Bacillus | BAB07799 | MSAIRVGIVGYGNLGRGVEFAISQNPDMELVAVFTRRDPSTV | 65 |
| | sphaericus | | SVASNASVYLVDDAEKFQDDIDVMILCGGSATDLPEQGPHFA | |
| | | | QWFNTIDSFDTHAKIPEFFDAVDAAAQKSGKVSVISVGWDPG | |
| | | | LFSLNRVLGEAVLPVGTTYTFWGDGLSQGHSDAVRRIEGVKN | |
| | | | AVQYTLPIKDAVERVRNGENPELTTREKHARECWVVLEEGAD | |
| | | | APKVEQEIVTMPNYFDEYNTTVNFISEDEFNANHTGMPHGGF | |
| | | | VIRSGESGANDKQILEFSLKLESNPNFTSSVLVAYARAAHRL | |
| | | | SQAGEKGAKTVFDIPFGLLSPKSAAQLRKELL | |
| dtsR1 | Thermobifida | ZP_00058587 | MATQAPEPLPADQIDIRTTAGKLADLQRRRYEAVHAGSEPAV | 66 |
| | fusca | | AKQHAKGKMTARERIDALLDPGSFVEFDAFARHRSTNFGLEK | |
| | | | NRPYGDGVVTGYGTIDGRPVAVFSQDVTVFGGSLGEVYGEKI | |
| | | | VKVLDHALKTGCPVIGINEGGGARIQEGVVALGLYAEIFKRN | |
| | | | THASGVIPQISLVMGAAAGGHVYSPALTDFIVMVDQTSQMFI | |
| | | | TGPDVIKTVTGEDVTMEELGGARTHNTKSGVAHYMASDEHDA | |
| | | | LEYVKALLSYLPSNNLDEPPVEPVQVTLEVTEEDRELDTFIP | |
| | | | DSANQPYDMRRVIEHIVDDGEFLEVHELFAQNIIVGFGRVEG | |
| | | | HPVGVVANQPMNLAGCLDIDASEKAARFVRTCDAFNIPVLTL | |
| | | | VDVPGFLPGTDQEFGGIIRRGAKLLYAYAEATVPLVTIITRK | |
| | | | AFGGAYDVMGSKHLGADINLAWPTAQIAVMGAQGAVNILHRR | |
| | | | TLAAADDVEATRAQLIAEYEDTLLNPYSAAERGYVDSVIMPS | |
| | | | ETRTSVIKALRALRGKRKQLPPKKHGNIPL | |
| dtsR1 | Streptomyces | ADD28194 | SEPEEQQPDIHTTAGKLADLRRRIEEATHAGSAPAVEKQHAK | 67 |
| | coelicolor | | GKLTARERIDLLLDEGSFVELDEFARIRSTNFGLDANRPYGG | |
| | | | VVTGYGTVDGRPVAVFSQDFTVFGGALGEVYGQKIVKVMDFA | |
| | | | LKTGCPVVGINDSGGARIQEGVASLGAYGEIFRRNTHASGIP | |
| | | | QISLVVGPCAGGAVYSPAITDFTVMVDQTSHMFITGPDVIKT | |
| | | | VTGEDVGFEELGGARTHNSTSGVAHHMAGDEKDAVEYVKQLL | |
| | | | SYLPSNNLSEPPAFPEEADLAVTDEDAELDTIVPDSANQPYD | |
| | | | MHSVIEHVLDDAEFFETQPLFAPNILTGFGRVEGRPVGIANQ | |
| | | | PMQFAGCLDITASEKARFVRTCDAFNVPVLTFVDVPGFLPGV | |
| | | | DQEHDGIIRRGAKLIFAYAEATVPLITVITRKAFGGADVMGS | |
| | | | KHLGADLNLAWPTAQIAVMGAQGAVNILHRRTIADADDAEAT | |
| | | | RARLIQEYEDALLNPYTAAERGYVDAVIMPSDTRRIVRGLRQ | |
| | | | LRTKRESLPPKKHGNIPL | |
| dtsR1 | Mycobacterium | CAB07063 | MTSVTDRSAHSAERSTEHTIDIHTTAGKLAELHKRREESLHP | 68 |
| | tuberculosis | | VGEDAVEKVHAKGKLTARERIYALLDEDSFVELDALAKHRST | |
| | (use this to clone | | NFNLGEKRPLGDGVVTGYGTIDGRDVCIFSQDATVFGGSLGE | |
| | M. smegmatis | | VYGEKIVKVQELAIKTGRPLIGINDGAGARIQEGVVSLGLYS | |
| | gene) | | RIFRNNILASGVIPQISLIMGAAAGGHVYSPALTDFVIMVDQ | |
| | | | TSQMFITGPDVIKTVTGEEVTMEELGGAHTHMAKSGTAHYAA | |
| | | | SGEQDAFDYVRELLSYLPPNNSTDAPRYQAAAPTGPIEENLT | |
| | | | DEDLELDTLIPDSPNQPYDMHEVITRLLDDEFLEIQAGYAQN | |
| | | | IVVGFGRIDGRPVGIVANQPTHFAGCLDINASEKAARFVRTC | |
| | | | DCFNIPIVMLVDVPGFLPGTDQEYNGIIRRGAKLLYAYGEAT | |
| | | | VPKITVITRKAYGGAYCVMGSKDMGCDVNLAWPTAQIAVMGA | |
| | | | SGAVGFVYRQQLAEAAANGEDIDKLRLRLQQEYEDTLVIPYV | |
| | | | AAERGYVDAVIPPSHTRGYIGTALRLLERKIAQLPPKKHGNV | |
| | | | PL | |
| dtsR1 | Mycobacterium | AAA85917 | MTSVTDHSAHSMERAAEHTINIHTTAGKLAELHKRTEEALHP | 69 |
| | leprae (use this | | VGAAAFEKVHAKGKFTARERIYALLDDDSFVELDALARHRST | |
| | to clone M. | | NFGLGERPVGDGVVTGYGTIDGRDVCIFSQDVTVFGGSLGEV | |
| | smegmatis | | YGEKIVKVQELAIKTGRPLIGINDGAGARIQEGVVSLGLYSR | |
| | gene) | | IFRNNILASGVIPQISLIMGAAAGGHVYSPALTDFVVMVDQT | |
| | | | SQMFITGPDVIKTVTGEDVTMEELGGAHTHMAKSGTAHYVAS | |
| | | | GEQDAFDWVRDVLSYLPSNNFTDAPRYSKPVPHGSIEDNLTA | |
| | | | KDLELDTLIPDSPNQPYDMHEVVTRLLDEEEFLEVQAGYATN | |
| | | | IVVGLGRIDDRPVGIVANQPIQFAGCLDINASEKAARFVRVC | |
| | | | DCFNIPIVMLVDVPGFLPGTEQEYDGIIRRGAKLLFAYGEAT | |
| | | | VPKITVITRKAYGGAYCVMGSKNMGCDVNLAWPTAQIAVMGA | |
| | | | SGAVGFVYRKELAQAAKNGANVDELRLQLQQEYEDTLVNPYI | |
| | | | AAERGYVDAVIPPSHTRGYIATALHLLERKIAHLPPKKHGNI | |
| | | | PL | |
| dtsR1 | Coryne- | NP_599940 | MTISSPLIDVANLPDINTTAGKIADLKARRAEANFPMGEKAV | 221 |
| | bacterium | | EKVHAAGRLTARERLDYLLDEGSFIETDQLARHRTTAFGLGA | |
| | glutamicum | | KRPATDGIVTGWGTIDGREVCIFSQDGTVFGGALGEVYGEKM | |
| | | | IKIMELAIDTGRPLIGLYEGAGARIQDGAVSLDFISQTFYQN | |
| | | | IQASGVIPQISVIMGACAGGNAYGPALTDFVVMVDKTSKMFV | |
| | | | TGPDVIKTVTGEEITQEELGGATTHMVTAGNSHYTAATDEEA | |
| | | | LDWVQDLVSFLPSNNRSYAPMEDFDEEEGGVEENITADDLKL | |
| | | | DEIIPDSATVPYDVRDVIECLTDDGEYLEIQADRAENVVIAF | |
| | | | GRIEGQSVGFVANQPTQFAGCLDIDSSEKAARFVRTCDAFNI | |
| | | | PIVMLVDVPGFLPGAGQEYGGILRRGAKLLYAYGEATVPKIT | |
| | | | VTMRKAYGGAYCVMGSKGLGSDINLAWPTAQIAVMGAAGAVG | |
| | | | FIYRKELMAADAKGLDTVALAKSFEREYEDHMLNPYHAAERG | |
| | | | LIDAVILPSETRGQISRNLRLLKHKNVTRPARKHGNNPL | |
| metH | Thermobifida | ZP_00059561 | MSARLSFREVLGSRVLVADGAMGTMLQTYDLSMDDFEGHEGC | 70 |
| | fusca | | NEVLNITRPDVVREIHEAYLQAGVDCVETNTFGANFGNLGEY | |
| | | | GIAERTYELAEAGARLAREAADAYTTADHVRYVLGSVGPGTK | |
| | | | LPTLGHAPYAVLRDHYEQCARGLIDGGVDAIVIETCQDLLQA | |
| | | | KAAIVGARPARKAAGTDTPIIVQVTIETTGTMLVGSEIGAAL | |
| | | | TSLEPLGVDMIGLNCATGPAEMSEHLRYLSHHSRIPLSCMPN | |
| | | | AGLPELGADGAVYPLQPHELTEAHDTFIREFGLALVGGCCGT | |
| | | | TPEHLAQVVERVQGRGVPDRKPHVEPAAASIYQSVPFRQDTS | |
| | | | YLAIGERTNANGSKAFREANLAERYDDCVEIARQQIRDGAHM | |
| | | | LDLCVDYVGRDGVRDMRELASRLATASTLPLVLDSTEVAVLE | |
| | | | AGLEMLGGRAVLNSVNYEDGDGPDSRFAKVAALAVEHGAALM | |
| | | | ALTIDEQGQARTAERKVEVAERLIRQLTTEYGIRKHDIIVDC | |
| | | | LTFTIATGQEESRRDALETIEAIRELKRRHPDVQTTLGVSNV | |
| | | | SFGLNPAARIVLNSVFLHECVQAGLDSAIVHASKILPINRIP | |
| | | | EEQRQVALDMIYDRRTDDYDPLQRFLQLFEGVDAQAMRASRE | |
| | | | EELAALPLWERLERRIVDGEAAGMEADLDEALTQRSALDIIN | |
| | | | TTLLAGMKTVGDLFGSGQMQLPFVLKSAEVMKAAVAYLEPHM | |
| | | | EKVDGDLGKGRIVLATVKGDVHDIGKNLVDIILSNNGYEVIN | |
| | | | LGIKQPISAILEAAERHRADVIGMSGLLVKSTVVMRENLEEM | |
| | | | NARGVADRYPVLLGGAALTRSYVEQDLAEIFKGEVRYARDAF | |
| | | | EGLKLMDAIMAVKRGVKGAKLPPLRTRRVKRGAQLTVTEPEK | |
| | | | MPTRSDVATDNPVPTPPFWGDRICKGIPLADYAAFLDERATF | |
| | | | MGQWGLRGSRGDGPTYEELVETEGRPRLRMWLDRIQTEGWLE | |
| | | | PAVVYGYYRCYSEGNDLVVLGEDENELTRFTFPRQRRDRNLC | |
| | | | LADFFRPKESGELDTVAFQVVTVGSTISKATAELFEKNAYRD | |
| | | | YLELHGLSVQLTEALAEYWHTRVRAELGFAGEDPDPADLDAY | |
| | | | FKLGYRGARFSLGYGACPNLEDRAKIVALLRPERVGVTLSEE | |
| | | | FQLVPEQSTDAIVVHHPEAKYFNV | |
| metH | Streptomyces | CAC18788 | MASSPSTPPADTRTRVSALREALATRVVVADGAMGTMLQAQN | 71 |
| | coelicolor | | PTLDDFQQLEGCNEVLNLTRPDIVRSVHEEYFAAGVDCVETN | |
| | | | TFGANHSALGEYDIPERVHELSEAGARVAREVADEFGARDGR | |
| | | | QRWVLGSMGPGTKLPTLGHAPYTVLRDAYQRNAEGLVAGGAD | |
| | | | ALLVETTQDLLQTKASVLGARRALDVLGLDLPLIVSVTVETT | |
| | | | GTMLLGSEIGAALTALEPLGIDMIGLNCATGPAEMSEHLRYL | |
| | | | ARHSRIPLTCMPNAGLPVLGKDGAHYPLTAPELADAHETFVR | |
| | | | EYGLSLVGGCCGTTPEHLRQVVERVRDTAPTARDPRPEPGAA | |
| | | | SLYQTVPFRQDTSYLAIGERTNANGSKKFREAMLDGRWDDCV | |
| | | | EMARDQIREGAHMLDLCVDYVGRDGVADMEELAGRFATASTL | |
| | | | PIVLDSTEVDVIRAGLEKLGGRAVINSVNYEDGAGPESRFAR | |
| | | | VTKLAREHGAALIALTIDEVGQARTAEKKVEIAERLIDDLTG | |
| | | | NWGIHESDILVDCLTFTICTGQEESRKDGLATIEGIRELKRR | |
| | | | HPDVQTTLGLSNISFGLNPAARILLNSVFLDECVKAGLDSAI | |
| | | | VHASKILPIARFDEEQVTTALDLIYDRRREGYDPLQKLMQLF | |
| | | | EGATAKSLKASKAEELAALPLEERLKRRIIDGEKNGLEQDLD | |
| | | | EALRERPALEIVNDTLLDGMKVVGELFGSGQMQLPFVLQSAE | |
| | | | VMKTAVAHLEPHMEKTDDDGKGTIVLATVRGDVHDIGKNLVD | |
| | | | IILSNNGYNVVNLGIKQPVSAILEAADEHRADVIGMSGLLVK | |
| | | | STVIMKENLEELNQRKLAADYPVILGGAALTRAYVEQDLHEI | |
| | | | YDGEVRYARDAFEGLRLMDALIGIKRGVPGAKLPELKQRRVR | |
| | | | AATVEIDERPEEGHVRSDVATDNPVPTPPFRGTRVVKGIQLK | |
| | | | EYASWLDEGALFKGQWGLKQARTGEGPSYEELVESEGRPRLR | |
| | | | GLLDRLQTDNLLEAAVVYGYFPCVSKDDDLIVLDDDG~ERTR | |
| | | | FTFPRQRRGRRLCLADFFRPEESGETDVVGFQVVTVGSRIGE | |
| | | | ETARMFEANAYRDYLELHGLSVQLAEALAEYWHARVRSELGF | |
| | | | AGEDPAEMEDMFALKYRGARFSLGYGACPDLEDPAKIAALLE | |
| | | | PERIGVHLSEEFQLHPEQSTDAIVIHHPEAKYFNAR | |
| metH | Mycobacterium | CAB10719 | MTAADKHLYDTDLLDVLSQRVMVGDGANGTQLQAADLTLDDF | 72 |
| | tuberculosis (use | | RGLEGCNEILNETRPDVLETIHRNYFEAGADAVETNTFGCNL | |
| | this to clone M. | | SNLGDYDIADRIRDLSQKGTAIARRVADELGSPDRKRYVLGS | |
| | smegmatis | | MGPGTKLPTLGHTEYAVIRDAYTEAALGMLDGGADAILVETC | |
| | gene) | | QDLLQLKAAVLGSRRANTRAGRHIPVFAHVTVETTGTMLLGS | |
| | | | EIGAALTAVEPLGVDMIGLNCATGPAEMSEHLRHLSRHARIP | |
| | | | VSVMPNAGLPVLGAKGAEYPLLPDELAEALAGFIAEFGLSLV | |
| | | | GGCCGTTPAHIREVAAAVANIKRPERQVSYEPSVSSLYTAIP | |
| | | | FAQDASVLVIGERTNANGSKGFREAMIAEDYQKCLDIAKDQT | |
| | | | RDGAHLLDLCVDYVGRDGVADMKALASRLATSSTLPIMLDST | |
| | | | ETAVLQAGLEHLGGRCAINSVNYEDGDGPESRFAKTMALVAE | |
| | | | HGAAVVALTIDEEGQARTAQKKVEIAERLINDITGNWGVDES | |
| | | | SILIDTLTFTIATGQEESRRDGIETIEAIRELKKRHPDVQTT | |
| | | | LGLSNISFGLNPAARQVLNSVFLHECQEAGLDSAIVHASKIL | |
| | | | PMNRIPEEQRNVALDLVYDRRREDYDPLQELMRLFEGVSAAS | |
| | | | SKEDRLAELAGLPLFERLAQRIVDGERNGLDADLDEANTQKP | |
| | | | PLQIINEHLLAGMKTVGELFGSGQMQLPFVLQSAEVMKAAVA | |
| | | | YLEPHMERSDDDSGKGRIVLATVKGDVHDIGKNLVDIILSNN | |
| | | | GYEVVNIGIKQPIATILEVAEDKSADVVGMSGLLVKSTVVMK | |
| | | | ENLEEMNTRGVAEKFPVLLGGAALTRSYVENDLAEIYQGEVH | |
| | | | YARDAFEGLKLMDTIMSAKRGEAPDENSPEAIKAREKEAERK | |
| | | | ARHQRSKRIAAQRKAAEEPVEVPERSDVAADIEVPAPPFWGS | |
| | | | RIVKGLAVADYTGLLDERALFLGQWGLRGQRGGEGPSYEDLV | |
| | | | ETEGRPRLRYWLDRLSTDGILAHAAVVYGYFPAVSEGNDIVV | |
| | | | LTEPKPDAPVRYRFHFPRQQRGRFLCIADFIRSRELAAERGE | |
| | | | VDVLPFQLVTMGQPIADFANELFASNAYRDYLEVHGIGVQLT | |
| | | | EALAEYWHRRIREELKFSGDRAMAAEDPEAKEDYFKLGYRGA | |
| | | | RFAFGYGACPDLEDRAKMMALLEPERIGVTLSEELQLHPEQS | |
| | | | TDAFVLHHPEAKYFNV | |
| metH | Mycobacterium | AA17182.1 | MRVTAANQHQYDTDLLETLAQRVMVGDGAMGTQLQDAELTLD | 73 |
| | leprae (use this | | DFRGLEGCNEILNETRPDVLETIHRRYFEAGADLVETNTFGC | |
| | to clone M. | | NLSNLGDYDIADKIRDLSQRGTVIARRVADELTTPDHKRYVL | |
| | smegmatis | | GSMGPGTKLPTLGHTEYRVVRDAYTESALGMLDGGADAVLVE | |
| | gene) | | TCQDLLQLKAAVLGSRRANTQAGRHIPVFVHVTVETTGTMLL | |
| | | | GSEIGAALAAVEPLGVDMIGLNCATGPAEMSEHLRHLSKHAR | |
| | | | IPVSVMPNAGLPVLGAKGAEYPLQPDELAEALAGFIAEFGLS | |
| | | | LVGGCCGTTPDHIREVAAAVARCNDGTVPRGERHVTYEPSVS | |
| | | | SLYTAIPFAQKPSVLMIGERTNANGSKVFREANIAEDYQKCL | |
| | | | DIAKDQTRGGAHLLDLCVDYVGRNGVADMKALAGRLATVSTL | |
| | | | PIMLDSTEIPVLQAGLEHLGGRCVUJSVNYEDGDGPESRFVK | |
| | | | TMELVAEHGAAVVALTIDEQGQARTVEKKVEVAERLINDITS | |
| | | | NWGVDKSAILIDCLTFTIATGQEESRKDGIETIDAIRELKKR | |
| | | | HPAVQTTLGLSNISFGLNPSARQVLNSVFLHECQEAGLDSAI | |
| | | | VHASKILPINRIPEEQRQAALDLVYDRRREGYDPLQKLMWLF | |
| | | | KGVSSPSSKETREAELAKLPLFDRLAQRIVDGERNGLDVDLD | |
| | | | EAMTQKPPLAIINENLLDGMKTVGELFGSGQMQLPFVLQSAE | |
| | | | VMKAAVAYLEPHMEKSDCDFGKGLAKGRIVLATVKGDVHDIG | |
| | | | KNLVDIILSNNGYEVVNLGIKQPITNILEVAEDKSADVVGMS | |
| | | | GLLVKSTVIMKENLEEMNTRGVAEKFPVLLGGAALTRSYVEN | |
| | | | DLAEVYEGEVHYARDAFEGLKLMDTIMSAKRGEALAPGSPES | |
| | | | LAAEADRNKETERKARHERSKRIAVQRKAAEEPVEVPERSDV | |
| | | | PSDVEVPAPPFWGSRIIKGLAVADYTGFLDERALFLGQWGLR | |
| | | | GVRGGAGPSYEDLVQTEGRPRLRYWLDRLSTYGVLAYAAVVY | |
| | | | GYFPAVSEDNDIVVLAEPRPDAEQRYRFTFPRQQRGRFLCIA | |
| | | | DFIRSRDLATERSEVDVLPFQLVTMGQPIADFVGELFVSNSY | |
| | | | RDYLEVHGIGVQLTEALAEYWHRRIREELKFSGNRTMSADDP | |
| | | | EAVEDYFKLGYRGARFAFGYGACPDLEDRIKMMELLQPERIG | |
| | | | VTISEELQLHPEQSTDAFVLHHPAAKYFNV | |
| metH | Lactobacillus | CAD63851 | MKFKQALQQRVLVADGAMGTLLYGNYGINSAFENLNLTHPDT | 74 |
| | plantarum | | ILRVHRSYIPAGADIIQTNTYAANRLKLTRYDLQDQVTTINQ | |
| | | | AAVKIAATAREHADHPVYILGTIGGLAGDTDATVQRATPATI | |
| | | | AASVTEQLTALLATNQLDGILLETYYDLPELLAALKIVKAHT | |
| | | | DLPVITNVSMLAPGVLRNGTSFTDAIVQLNAAGADVIGTNCR | |
| | | | LGPYYLAQSFENLAIPANVKLAVYPNAGLPGTDQDGAVVYDG | |
| | | | EPSYFEEYAERFRQLGLNIIGGCCGTTPLHTSATVRGLSNRS | |
| | | | IVAHDQPATKPQPPTLVTTKSQHRFLQKVATQKTALVELDPP | |
| | | | RDFDTTKFFRGAERLKAAGVDGITLSDNSLATVRIANTTIAA | |
| | | | QLKLNYGITPIVHLTTRDHNLIGLQSEIMGLHSLGIEDILAI | |
| | | | TGDPAKLGDFPGATSVSDVRSVELMKLIKQFNSGIGPTGKSL | |
| | | | KEASDFRVAGAFNPNAYRTSISTKSISRKLSYGCDYIITQPV | |
| | | | YDLANVDALADALAANHVNVPVFVGVMPLVSRRNAEFLHHEV | |
| | | | HGIRIPEPILTRMAEAEQTGNERAVGIAIAKELIDGICARFN | |
| | | | GVHIVTPFNRFKTVIELVDYIQQKNLIKVQ | |
| metH | Coryne- | CAD26709 | MSTSVTSPAHNNAHSSEFLDALANHVLIGDGAMGTQLQGFDL | 222 |
| | bacterium | | DVEKDFLDLEGCNEILNDTRPDVLRQIHRAYFEAGADLVETN | |
| | glutamicum | | TFGCNLPNLADYDIADRCRELAYKGTAVAREVADEMGPGRNG | |
| | | | MRRFVVGSLGPGTKLPSLGHAPYADLRGHYKEAAWGIIDGGG | |
| | | | DAFLIETAQDLLQVKAAVHGVQDANAELDTFLPIICHVTVET | |
| | | | TGTNLMGSEIGAALTALQPLGIDMIGLNCATGPDEMSEHLRY | |
| | | | LSKHADIPVSVMPNAGLPVLGKNGAEYPLEAEDLAQALAGFV | |
| | | | SEYGLSMVGGCCGTTPEHIRAVRDAVVGVPEQETSTLTKIPA | |
| | | | GPVEQASREVEKEDSVASLYTSVPLSQETGISMIGERTNSNG | |
| | | | SKAFREAMLSGDWEKCVDIAKQQTRDGAHMLDLCVDYVGRDG | |
| | | | TADMATLAALLATSSTLPIMIDSTEPEVIRTGLEHLGGRSIV | |
| | | | NSVNFEDGDGPESRYQRIMKLVKQHGAAVVALTIDEEGQART | |
| | | | AEHKVRIAKRLIDDITGSYGLDIKDIVVDCLTFPISTGQEET | |
| | | | RRDGIETIEAIRELKKLYPEIHTTLGLSNISFGLNPAARQVL | |
| | | | NSVFLNECIEAGLDSAIAHSSKILPMNRIDDRQREVALDMVY | |
| | | | DRRTEDYDPLQEFMQLFEGVSAADAKDAPAEQLAAMPLFERL | |
| | | | AQRIIDGDKRGLEDDLEAGMKEKSPIAIINEDLLNGMKTVGE | |
| | | | LFGSGQMQLPFVLQSAETMKTAVAYLEPFMEEEAEATGSAQA | |
| | | | EGKGKIVVATVKGDVHDIGKNLVDIILSNNGYDVVNLGIKQP | |
| | | | LSAMLEAAEEHKADVIGMSGLLVKSTVVMKENLEEMNNAGAS | |
| | | | NYPVILGGAALTRTYVENDLNEVYTGEVYYARDAFEGLRLMD | |
| | | | EVMAEKRGEGLDPNSPEAIEQAKKKAERKARNERSRKIAAER | |
| | | | KANAAPVIVPERSDVSTDTPTAAPPFWGTRIVKGLPLAEFLG | |
| | | | NLDERALFMGQWGLKSTRGNEGPSYEDLVETEGRPRLRYWLD | |
| | | | RLKSEGILDHVALVYGYFPAVAEGDDVVILESPDPHAAERMR | |
| | | | FSFPRQQRGRFLCIADFIRPREQAVKDGQVDVMPFQLVTMGN | |
| | | | PIADFANELFAANEYREYLEVHGIGVQLTEALAEYWHSRVRS | |
| | | | ELKLNDGGSVADFDPEDKTKFFDLDYRGARFSFGYGSCPDLE | |
| | | | DRAKLVELLEPGRIGVELSEELQLHPEQSTDAFVLYHPEAKY | |
| | | | FNV | |
| metH | Escherichia coli | P13009 | MSSKVEQLPAQLNERILVLDGGMGTMIQSYRLNEADFRGERF | 223 |
| | | | ADWPCDLKGNNDLLVLSKPEVIAAIHNAYFEAGADIIETNTF | |
| | | | NSTTIAMADYQMESLSAEINFAAAKLARRCADEWTARTPEKP | |
| | | | RYVAGVLGPTNRTASISPDVNDPAFRNITFDGLVAAYRESTK | |
| | | | ALVEGGADLILIETVFDTLNAKAAVFAVKTEFEALGVELPIM | |
| | | | ISGTITDASGRTLSGQTTEAFYNSLRHAEALTFGLNCALGPD | |
| | | | ELRQYVQELSRIAECYVTAHPNAGLPNAFGEYDLDADTMAKQ | |
| | | | IREWAQAGFLNIVGGCCGTTPQHIAAMSRAVEGLAPRKLPEI | |
| | | | PVACRLSGLEPLNIGEDSLFVNVGERTNVTGSAKFKRLIKEE | |
| | | | KYSEALDVARQQVENGAQIIDINMDEGMLDAEAAMVRFLNLI | |
| | | | AGEPDIARVPIMIDSSKWDVIEKGLKCIQGKGIVNSISMKEG | |
| | | | VDAFIHHAKLLRRYGAAVVVMAFDEQGQADTRARKIEICRRA | |
| | | | YKILTEEVGFPPEDIIFDPNIFAVATGIEEHNNYAQDFIGAC | |
| | | | EDIKRELPHALISGGVSIVSFSFRGNDPVREAIHAVFLYYAI | |
| | | | RNGMDMGIVNAGQLAIYDDLPAELRDAVEDVILNRRDDGTER | |
| | | | LLELAEKYRGTKTDDTANAQQAEWRSWEVNKRLEYSLVKGIT | |
| | | | EFIEQDTEEARQQATRPIEVIEGPLMDGMNVVGDLFGEGKMF | |
| | | | LPQVVKSARVMKQAVAYLEPFIEASKEQGKTNGKMVIATVKG | |
| | | | DVHDIGKNIVGVVLQCNNYEIVDLGVMVPAEKILRTAKEVNA | |
| | | | DLIGLSGLITPSLDEMVNVAKEMERQGFTIPLLIGGATTSKA | |
| | | | HTAVKIEQNYSGPTVYVQNASRTVGVVAALLSDTQRDDFVAR | |
| | | | TRKEYETVRIQHGRKKPRTPPVTLEAARDNDFAFDWQAYTPP | |
| | | | VAHRLGVQEVEASIETLRNYIDWTPFFMTWSLAGKYPRILED | |
| | | | EVVGVEAQRLFKDANDMLDKLSAEKTLNPRGVVGLFPANRVG | |
| | | | DDIEIYRDETRTHVINVSHHLRQQTEKTGFANYCLADFVAPK | |
| | | | LSGKADYIGAFAVTGGLEEDALADAFEAQHDDYNKIMVKALA | |
| | | | DRLAEAFAEYLHERVRKVYWGYAPNENLSNEELIRENYQGIR | |
| | | | PAPGYPACPEHTEKATIWELLEVEKHTGMKLTESFAMWPGAS | |
| | | | VSGWYFSHPDSKYYAVAQIQRDQVEDYARRKGMSVTEVERWL | |
| | | | APNLGYDAD | |
| metE | Mycobacterium | CAB09044 | MTQPVRRQPFTATITGSPRIGPRRELKPATEGYWAGRTSRSE | 75 |
| | tuberculosis (use | | LEAVAATLRRDTWSALAAAGLDSVPVNTFSYYDQMLDTAVLL | |
| | this to clone M. | | GALPPRVSPVSDGLDRYFAAARGTDQIAPLEMTKWFDTNYHY | |
| | smegmatis | | LVPEIGPSTTFTLHPGKVLAELKEALGQGIPARPVIIGPITF | |
| | gene) | | LLLSKAVDGAGAPIERLEELVPVYSELLSLLADGGAQWVQFD | |
| | | | EPALVTDLSPDAPALAEAVYTALCSVSNRPAIYVATYFGDPG | |
| | | | AALPALARTPVEAIGVDLVAGADTSVAGVPELAGKTLVAGVV | |
| | | | DGRNVWRTDLEAALGTLATLLGSAATVAVSTSCSTLHVPYSL | |
| | | | EPETDLDDALRSWLAFGAEKVREVVVLARALRDGHDAVADEI | |
| | | | ASSRAAIASRKRDPRLHNGQIRAPIEAIVASGAHRGNAAQRR | |
| | | | ASQDARLHLPPLPTTTIGSYPQTSAIRVARAALPAGEIDEAE | |
| | | | YVRRMRQEITEVIALQERLGLDVLVHGEPERNDMVQYFAEQL | |
| | | | AGFFATQNGWVQSYGSRCVRPPILYGDVSRPRAMTVEWITYA | |
| | | | QSLTDKPVKGMLTGPVTILAWSFVRDDQPLADTANQVALAIR | |
| | | | DETVDLQSAGIAVIQVDEPALRELLPLRRADQAEYLRWAVGA | |
| | | | FRLATSGVSDATQIHTHLCYSEFGEVIGAIADLDADVTSTEA | |
| | | | ARSHMEVLDDLNAIGFANGVGPGVYDIHSPRVPSAEEMADSL | |
| | | | RAALRAVPAERLWVNPDCGLKTRNVDEVTASLHNMVAAAREV | |
| | | | RAG | |
| metE | Mycobacterium | CAB08123 | MDELVTTQSFTATVTGSPRIGPRRELKRATEGYWAKRTSRSE | 76 |
| | leprae (use this | | LESVASTLRRDMWSDLAAAGLDSVPVNTFSYYDQMLDTAFML | |
| | to clone M. | | GALPARVAQVSDDLDQYFALARGNNDIKPLEMTKWFDTNYHY | |
| | smegmatis | | LVPEIEPATTFSLNPGKILGELKEALEQRIPSRPVIIGPVTF | |
| | gene) | | LLLSKGINGGGAPIQRLEELVGIYCTLLSLLAENGARWVQFD | |
| | | | EPALVTDLSPDAPALAEAVYTALGSVSKRPAIYVATYFGNPG | |
| | | | ASLAGLARTPIEAIGVDFVCGADTSVAAVPELAGKTLVAGIV | |
| | | | DGRNIWRTDLESALSKLATLLGSAATVAVSTSCSTLHVPYSL | |
| | | | EPETDLDDNLRSWLAFGAEKVAEVVVLAPALRDGRDAVADEI | |
| | | | AASNAAVASRRSDPRLHNGQVRARIDSIVASGTHRGDAAQRR | |
| | | | TSQDARLHLPPLPTTTIGSYPQTSAIRKARAALQDAEIDEAE | |
| | | | YISRMKKEVADAIKLQEQLGLDVLVHGEPERNDMVQYFAEQL | |
| | | | GGFFATQNGWVQSYGSRCVRPPILYGDVSRPHPMTIEWITYA | |
| | | | QSLTDKPVKGMLTGPVTILAWSFVRDDQPLADTANQVALAIR | |
| | | | DETVDLQSAGIAIIQVDEPALRELLPLRRADQDEYLCWAVKA | |
| | | | FRLATSGVADSTQIHTHLCYSEFGEVIGAIADLDADVTSIEA | |
| | | | ARSHMEVLDDLNAVGFANSIGPGVYDIHSPRVPSTDEIAKSL | |
| | | | RAALKAIPMQRLWVNPDCGLKTRSVDEVSASLQNMVAAARQV | |
| | | | RAGA | |
| metE | Streptomyces | CAC44335 | MTAKSAAAAARATVYGYPRQGPNRELKKAIEGYWKGRVSAPE | 77 |
| | coelicolor | | LRSLAADLRAANWRRLADAGIDEVPAGDFSYYDHVLDTTVMV | |
| | | | GAIPERHRAAVAADALDGYFANARGTQEVAPLEMTKWFDTNY | |
| | | | HYLVPELGPDTVFTADSTKQVTELAEAVALGLTARPVLVGPV | |
| | | | TYLLLAKPAPGAPADFEPLTLLDRLLPVYAEVLTDLRAAGAE | |
| | | | WVQLDEPAFVQDRTPAELNALERAYRELGALTDRPKLLVASY | |
| | | | FDRLGDALPVLAKAPIEGLALDFTDAAATNLDALAAVGGLPG | |
| | | | KRLVAGVVNGRNIWINDLQKSLSTLGTLLGLADRVDVSASCS | |
| | | | LLHVPLDTGAERDIEPQILRWLAFARQKTAEIVTLAKGLAQG | |
| | | | TDAITGELAASRADMASRAGSPITRNPAVRARAEAVTDDDAR | |
| | | | RSQPYAERTAAQPAHLGLPPLPTTTIGSFPQTGEIRAARADL | |
| | | | RDGRIDIAGYEERIPAEIQEVISFQEKTGLDVLVHGEpERND | |
| | | | MVQYFAEQLTGYLATQHGWVQSYGTRYVRPPILAGDISRPEP | |
| | | | MTVRWTTYAQSLTEKPVKGMLTGPVTMLAWSFVRDDQPLGDT | |
| | | | ARQVALALRDEVNDLEAAGTSVIQVDEPALRETLPLPAADHT | |
| | | | AYLAWATEAFRLTTSGVRPDTQIHTHMCYAEFGDIVQAIDDL | |
| | | | DADVISLEAARSHMQVAHELATHGYPREAGPGVYDIHSPRVP | |
| | | | SAEEAAALLRTGLKAIPAERLWVNPDCGLKTRGWPETRASLE | |
| | | | NLVATARTLRGELSAS | |
| metE | Coryne- | CAD26711 | MTSNFSSTVAGLPRIGAKRELKFALEGYWNGSIEGRELAQTA | 224 |
| | bacterium | | RQLVNTASDSLSGLDSVPFAGRSYYDAMLDTAAILGVLPERF | |
| | glutamicum | | DDIADHENDGLPLWIDRYFGAARGTETLPAQAMTKWFDTNYH | |
| | | | YLVPELSADTRFVLDASALIEDLRCQQVRGVNARPVLVGPLT | |
| | | | FLSLARTTDGSNPLDHLPALFEVYERLIKSFDTEWVQIDEPA | |
| | | | LVTDVAPEVLEQVRAGYTTLAKRDGVFVNTYFGSGDQALNTL | |
| | | | AGIGLGAIGVDLVTHGVTELAAWKGEELLVAGIVDGRNIWRT | |
| | | | DLCAALASLKRLAARGPIAVSTSCSLLHVPYTLEAENIEPEV | |
| | | | RDWLAFGSEKITEVKLLADALAGNIDAAAFDAASAAIASRRT | |
| | | | SPRTAPITQELPGRSRGSFDTRVTLQEKSLELPALPTTTIGS | |
| | | | FPQTPSIRSARARLRKESITLEQYEEAMREEIDLVIAKQEEL | |
| | | | GLDVLVHGEPERNDMVQYFSELLDGFLSTANGWVQSYGSRCV | |
| | | | RPPVLFGNVSRPAPMTVKWFQYAQSLTQKEVKGMLTGPVTIL | |
| | | | AWSFVRDDQPLATTADQVALALRDEINDLIEAGAKIIQVDEP | |
| | | | AIRELLPLRDVDKPAYLQWSVDSFRLATAGAPDDVQIHTHMC | |
| | | | YSEFNEVISSVIALDADVTTIEAARSDMQVLAALKSSGFELG | |
| | | | VGPGVWDIHSPRVPSAQEVDGLLEAALQSVDPRQLWVNpDCG | |
| | | | LKTRGWPEVEASLKVLVESAKQAREKIGATI | |
| metE | Escherichia coli | Q8FBM1 | MTILNHTLGFPRVGLRRELKKAQESYWAGNSTREELLAVGRE | 225 |
| | | | LRARHWDQQKQAGIDLLPVGDFAWYDHVLTTSLLLGNVPPRH | |
| | | | QNKDGSVDIDTLFRIGRGRAPTGEPAAAAEMTKWFNTNYHYM | |
| | | | VPEFVKGQQFKLTWTQLLEEVDEALALGHKVKPVLLGPITYL | |
| | | | WLGKVKGEQFDRLSLLNDILPVYQQVLAELAKRGIEwVQIDE | |
| | | | PALVLELPQAWLDAYKPAYDALQGQVKLLLTTYFEGVTPNLD | |
| | | | TITALPVQGLHVDLVHGKDDVAELHKRLPSDWLLSAGLINGR | |
| | | | NVWRADLTEKYAQIKDIVGKRDLWVASSCSLLHSPIDLSVET | |
| | | | RLDAEVKSWFAFALQKCHELALLRDALNSGDTAALAEWSAPI | |
| | | | QARRHSTRVHNPAVEKRLAAITAQDSQRANVYEVRAEAQRAR | |
| | | | FKLPAWPTTTIGSFPQTTEIRTLRLDFKKGNLDANNYRTGIA | |
| | | | EHIKQAIVEQERLGLDVLVHGEAERNDMVEYFGEHLDGFVFT | |
| | | | QNGWVQSYGSRCVKPPIVIGDVSRPAPITVEWAKYAQSLTDK | |
| | | | PVKGMLTGPVTILCWSFPREDVSRETIAKQIALALRDEVADL | |
| | | | EAAGIGIIQIDEPALREGLPLRRSDWDAYLQWGVEAFRINAA | |
| | | | VAKDDTQIHTHMCYCEFNDIMDSIAALDADVITIETSRSDME | |
| | | | LLESFEEFDYPNEIGPGVYDIHSPNVPSVEWIEALLKKAAKR | |
| | | | IPAERLWVNPDCGLKTRGWPETRAALANMVQAAQNLRRG | |
| glyA | Streptomyces | CAA20173 | MSLLNTPLHELDPDVAAAVDAELDRQQSTLEMIASENFAPVA | 78 |
| | coelicolor | | VMEAQGSVLTNKYAEGYPGRRYYGGCEHVDVVEQIAIDRVKA | |
| | | | LFGAEHANVQPHSGAQANAAAMFALLKPGDTIMGLNLAHGGH | |
| | | | LTHGMKINFSGKLYNVVPYHVGDDGQVDMAEVERLAKETKPK | |
| | | | LIVAGWSAYPRQLDFAAFRKVADEVGAYLMVDMAHFAGLVAA | |
| | | | GLHPNPVPHAHVVTTTTHKTLGGPRGGVILSTAELAKKINSA | |
| | | | VFPGQQGGPLEHVVAAKAVAFKVAASEDFKERQGRTLEGARI | |
| | | | LAERLVRDDAKAAGVSVLTGGTDVHLVLVDLRDSELDGQQAE | |
| | | | DRLHEVGITVNRNAVPNDPRPPMVTSGLRIGTPALATRGFTA | |
| | | | EDFAEVADVIAEALKPSYDAEALKARVKTLADKHPLYPGLNK | |
| glyA | Thermobifide | ZP_00058615 | MKVRKLMTAQSTSLTQSLAQLDPEVAAAVDAELARQRDTLEM | 79 |
| | fusca | | IASENFAPPAVLEAQGTVLTNKYAEGYPGRRYYGGCEHVDVI | |
| | | | EQLAIDRAKALFGAEHANVQPHSGAQANTAVYFALLQPGDTI | |
| | | | LGLDLAHGGHLTHGMRINYSGKILNAVAYHVRESDGLIDYDE | |
| | | | VEALAKEHQPKLIIAGWSAYPRQLDFARFREIADQTGALLMV | |
| | | | DMAHFAGLVAAGLHPNPVPYADVVTTTTHKTLGGPRGGLILA | |
| | | | KEELGKKIMSAVFPGMQGGPLQHVIAAKAVALKVAASEEFAE | |
| | | | RQRRTLSGAKILAERLTQPDAAEAGIRVLTGGTDVHLVLVDL | |
| | | | VNSELNGKEAEDRLHEIGITVNRNAVPNDPRPPMVTSGLRIG | |
| | | | TPALATRGFGDADFAEVADIIAEALKPGFDAATLRSRVQALA | |
| | | | AKHPLYPGL | |
| glyA | Mycobacterium | AAK45383 | MSAPLAEVDPDIAELLAKELGRQRDTLEMIASENFAPRAVLQ | 80 |
| | tuberculosis (use | | AQGSVLThKYAEGLPGRRYYGGCEHVDVVENLARDRAKALFG | |
| | this to clone M. | | AEFANVQPHSGAQANAAVLHALMSPGERLLGLDLANGGHLTH | |
| | smegmatis | | GMRLHFSGKLYENGFYGVDPATHLIDMDAVPATALEFRPKVI | |
| | gene) | | IAGWSAYPRVLDFAAFRSIADEVGAKLLVDMAHFAGLVAAGL | |
| | | | HPSPVPHADVVSTTVHKTLGGGRSGLIVGKQQYAKAINSAVF | |
| | | | PGQQGGPLMHVIAGKAVALKIAATPEFADRQRRTLSGARIIA | |
| | | | DRLMAPDVAKAGVSVVSGGTDVHLVLVDLRDSPLDGQAAEDL | |
| | | | LHEVGITVNRNAVPNDPRPPMVTSGLRIGTPALATRGFGDTE | |
| | | | FTEVADIIATALATGSSVDVSALKDRATRLARAFPLYDGLEE | |
| | | | WSLVGR | |
| glyA | Mycobacterium | CAB39828 | MVAPLAEVDPDIAELLGKELGRQRDTLEMIASENFVPRSVLQ | 81 |
| | leprae (use this | | AQGSVLTNKYAEGLPGRRYYDGCEHVDVVENIARDRAKALFG | |
| | to clone M. | | ADFANVQPHSGAQANAAVLHALMSPGERLLGLDLANGGHLTH | |
| | smegmatis | | GMRLNFSGKLYETGFYGVDATTHLIDMDAVRAKALEFRPKVL | |
| | gene) | | IAGWSAYPRILDFAAFRSIADEVGAKLWVDMAHFAGLVAVGL | |
| | | | HPSPVPHADVVSTTVHKTLGGGRSGLILGKQEFATAINSAVF | |
| | | | PGQQGGPLMHVIAGKAVALKIATTPEFTDRQQRTLAGARILA | |
| | | | DRLTAADVTKAGVSVVSGGTDVHLVLVDLRNSPFDGQAAEDL | |
| | | | LHEVGITVNRNVVPNDPRPPMVTSGLRIGTPALATRGFGEAE | |
| | | | FTEVADIIATVLTTGGSVDVAALRQQVTRLARDFPLYGGLED | |
| | | | WSLAGR | |
| glyA | Lactobacillus | CAD64690 | MNYQEQDPEVWAAISKEQARQQHNIELIASEHIVSKGVRAAQ | 82 |
| | plantarum | | GSVLTNKYSEGYPGHRFYGGNEYIDQVETLAIERAKKLFGAE | |
| | | | YANVQPHSGSQANAAAYMALIQPGDRVMGMSLDAGGHLTHGS | |
| | | | SVNFSGKLYDFQGYGLDPETAELNYDAILAQAQDFQPKLIVA | |
| | | | GASAYSRLIDFKKFREIADQVGALLMVDMAHIAGLVAAGLHP | |
| | | | NPVPYADVVTTTTHKTLRGPRGGMILAKEKYGKKINSAVFPG | |
| | | | NQGGPLDHVIAGKAIALGEDLQPEFKVYAQHIIDNAKAMAKV | |
| | | | FNDSDLVRVISGGTDNHLMTIDVTKSGLNGRQVQDLLDTVYI | |
| | | | TVNKEAIPNETLGAFKTSGIRLGTPAITTRGFDEADATKVAE | |
| | | | LILQALQAPTDQANLDDVKQQAMALTAKHPIDVD | |
| glyA | Coryne- | AAK60516 | MTDAHQADDVRYQPLNELDPEVAAAIAGELARQRDTLEMIAS | 226 |
| | bacterium | | ENFVPRSVLQAQGSVLTNKYAEGYPGRRYYGGCEQVDIIEDL | |
| | glutamicum | | ARDRAKALFGAEFANVQPHSGAQANAAVLMTLAEPGDKIMGL | |
| | | | SLAHGGHLTHGMKLNFSGKLYEVVAYGVDPETMRVDMDQVRE | |
| | | | IALKEQPKVIIAGWSAYPRHLDFEAFQSIAAEVGAKLWVDMA | |
| | | | HFAGLVAAGLHPSPVPYSDVVSSTVHKTLGGPRSGIILAKQE | |
| | | | YAKKLNSSVFPGQQGGPLMHAVAAKATSLKIAGTEQFRDRQA | |
| | | | RTLEGARILAERLTASDAKAAGVDVLTGGTDVHLVLADLRNS | |
| | | | QMDGQQAEDLLHEVGITVNRNAVPFDPRPPMVTSGLRIGTPA | |
| | | | LATRGFDIPAFTEVADIIGTALANGKSADIESLRGRVAKLAA | |
| | | | DYPLYEGLEDWTIV | |
| glyA | Escherichia coli | P00477 | MLKREMNIADYDAELWQAMEQEKVRQEEHIELIASENYTSPR | 227 |
| | | | VMQAQGSQLTNKYAEGYPGKRYYGGCEYVDIVEQLAIDRAKE | |
| | | | LFGADYANVQPHSGSQANFAVYTALLEPGDTVLGMNLAHGGH | |
| | | | LTHGSPVNFSGKLYNIVPYGIDATGHIDYADLEKQAKEHKPK | |
| | | | MIIGGFSAYSGVVDWAKMREIADSIGAYLFVDMAHVAGLVAA | |
| | | | GVYPNPVPHAHVVTTTTHKTLAGPRGGLILAKGGSEELYKKL | |
| | | | NSAVFPGGQGGPLMHVIAGKAVALKEAMEPEFKTYQQQVAKN | |
| | | | AKAMVEVFLERGYKVVSGGTDNHLFLVDLVDKNLTGKEADAA | |
| | | | LGRANITVNKNSVPNDPKSPFVTSGIRVGTPAITRRGFKEAE | |
| | | | AKELAGWMCDVLDSINDEAVIERIKGKVLDICARYPVYA | |
| metE | Thermobifida | ZP_00056753 | MASRAASTGSHSAPISSSSGRRLATKAASSASTRGRTKATGD | 83 |
| | fusca | | KCEELIRAGYRLFRRPSSPRHTQTPPIWSITVGDMLGSPTPR | |
| | | | PAPRPRRISELLARKEPTFSFEFFPPKTPEGERMLWRAIREI | |
| | | | EALRPSFVSVTYGAGGSTRDRTVNVTEKIATNTTLLPVAHIT | |
| | | | AVNHSVRELRHLIGRFAAAGVCNMLAIRGDPPGDPLGEWVKH | |
| | | | PEGLTHAEELVRLIKESGDFCVGVAAFPYKHPRSPDVETDTD | |
| | | | FFVRKCRAGADYAITQMFFEAEDYLRLRDRVAARGCDVPIIP | |
| | | | EIMPVTKFSTIARSEQLSGAPFPRRLAEEFERVADDPEAVRA | |
| | | | LGIEHATRLCERLLAEGAPGIHFITFNRSTATREVYHRLVGA | |
| | | | TQPAAVAALP | |
| metF | Streptomyces | CAB52012 | MALGTASTRTDPARTVRDILATGKTTYSFEFSAPKTPKGERN | 84 |
| | coelicolor | | LWSALRRVEAVAPDFVSVTYGAGGSTRAGTVRETQQIVADTT | |
| | | | LTPVAHLTAVDHSVAELRNIIGQYADAGIRNMLAVRGDPPGD | |
| | | | PNADWIAHPEGLTYAAELVRLIKESGDFCVGVAAFPEMHPRS | |
| | | | ADWDTDVTNFVDKCRAGADYAITQMFFQPDSYLRLRDRVAAA | |
| | | | GCATPVIPEVMPVTSVKMLERLPKLSNASFPAELKERILTAK | |
| | | | DDPAAVRSIGIEFATEFCARLLAEGVPGLHFITLNNSTATLE | |
| | | | IYENLGLHHPPPA | |
| metE | Coryne- | CAD26762 | MVEVNKCQRQSQQNTLITLRYPGMSLTNIPASSQWAISDVLK | 228 |
| | bacterium | | RPSPGRVPFSVEFMPPRDDAAEERLYRAAEVFHDLGASFVSV | |
| | glutamicum | | TYGAGGSTRERTSRIARRLAKQPLTTLVHLTLVNBTREEMKA | |
| | | | ILREYLELGLTNLLALRGDPPGDPLGDWVSTDGGLNYASELI | |
| | | | DLIKSTPEFREFDLGIASFPEGHFRAKTLEEDTKYTLAKLRG | |
| | | | GAEYSITQMFFDVEDYLRLRDRLVAADPIHGAKPIIPGIMPI | |
| | | | TELRSVRRQVELSGAQLPSQLEESLVRAANGNEEANKDEIRK | |
| | | | VGIEYSTNMAERLIAEGAEDLHFMTLNFTRATQEVLYNLGMA | |
| | | | PAWGAEHGQDAVR | |
| metF | Escherichia coli | NP_418376 | MSFFHASQRDALNQSLAEVQGQINVSFEFFPPRTSEMEQTLW | 229 |
| | | | NSIDRLSSLKPKFVSVTYGANSGERDRTHSIIKGIKDRTGLE | |
| | | | AAPHLTCIDATPDELRTIARDYWNNGIRHIVALRGDLPPGSG | |
| | | | KPEMYASDLVTLLKEVADFDISVAAYPEVHPEAKSAQADLLN | |
| | | | LKRKVDAGANPAITQFFFDVESYLRFRDRCVSAGIDVEIIPG | |
| | | | ILPVSNFKQAKKFADMTNVRIPAWMAQMFDGLDDDAETRKLV | |
| | | | GANIAMDMVKILSREGVKDFHFYTLNRAEMSYAICHTLGVRP | |
| | | | GL | |
| cysE | Mycobacterium | K46690 | MLTAMRGDIRAARERDPAAPTALEVIFCYPGVHAVWGHRLAH | 85 |
| | tuberculosis (use | | WLWQRGARLLAPAAAEFTRILTGVDIHPGAVIGARVFIDHAT | |
| | this to clone M. | | GVVIGETAEVGDDVTIYHGVTLGGSGMVGGKRHPTVGDRVII | |
| | smegmatis | | GAGAKVLGPIKIGEDSRIGANAVVVKPVPPSAVVVGVPGQVI | |
| | gene) | | GQSQPSPGGPFDWRLPDLVGASLDSLLTRVARLDALGGGPQA | |
| | | | AGVIRPPEAGIWHGEDFSI | |
| cysE | Mycobacterium | CAB11413 | MFAAIRRDIQAARQRDPAQPTVLEVICCYPGVHAVWGHRISH | 86 |
| | leprae (use this | | WLWNRRARLAARAFAELTRILTGVDIHPGAVLGAGLFIDHAT | |
| | to clone M. | | GVVIGETAEVGDDVTIFHGVTLGGTGRETGKRHPTIGDRVTI | |
| | smegmatis | | GAGAKVLGAIKIGEDSRIGANAVVVKEVPASAVAVGVPGQII | |
| | gene) | | SSDSPANGDDSVLPDFVGVSLQSLLTRVAKLEAEDGGSQTYR | |
| | | | VIRLPEAGVWHGEDFSI | |
| cysE | Lactobacillus | CAD62911 | MFQTARAILNRDPAAINLRTVMLTYPGIHALAWYRVAHYFET | 87 |
| | plantarum | | HRLPLLAALLSQHAARHTGILIHPAAQIGHRVFFDHGIGTVI | |
| | | | GATAVIEDDVTILHGVTLGARKTEQAGRRHPYVCRGAFIGAH | |
| | | | AQLLGPITIGANSKIGAGAIVLDSVPAHVTAVGNPAHLVATQ | |
| | | | LHAYHEATSNQA | |
| cysE | Coryne- | CAD34661 | MLSTIKMIREDLANAREHDPAARGDLENAVVYSGLHAIWAHR | 230 |
| | bacterium | | VANSWWKSGFRGPARVLAQFTRFLTGIEIHPGATIGRRFFID | |
| | glutamicum | | HGMGIVIGETAEIGEGVMLYHGVTLGGQVLTQTKRHPTLCDN | |
| | | | VTVGAGAKILGPITIGEGSAIGANAVVTKDVPAEHIAVGIPA | |
| | | | VARPRGKTEKIKLVDPDYYI | |
| cysE | Escherichia coli | NP_418064 | MSCEELEIVWNNIKAEARTLADCEPMLASFYHATLLKHENLG | 231 |
| | | | SALSYMLANKLSSPIMPAIAIREVVEEAYAADPEMIASAACD | |
| | | | IQAVRTRDPAVDKYSTPLLYLKGFHALQAYRIGHWLWNQGRR | |
| | | | ALAIFLQNQVSVTFQVDIHPAAKIGRGIMLDHATGIVVGETA | |
| | | | VIENDVSILQSVTLGGTGKSGGDRHPKIREGVMIGAGAKILG | |
| | | | NIEVGRGAKIGAGSVVLQPVPPHTTAAGVPARIVGKPDSDKP | |
| | | | SMDMDQHFNGINHTFEYGDGI | |
| serA | Mycobacterium | CAA16081 | MSLPVVLIADKLAPSTVAALGDQVEVRWVDGPDRDKLLAAVP | 88 |
| | tuberculosis (use | | EADALLVRSATTVDAEVLAAAPKLKIVARAGVGLDNVDVDAA | |
| | this to clone M. | | TARGVLVVNAPTSNIHSAAEHALALLLAASRQIPAADASLRE | |
| | smegmatis | | HTWKRSSFSGTEIFGKTVGVVGLGRIGQLVAQRIAAFGAYVV | |
| | gene) | | AYDPYVSPARAAQLGIELLSLDDLLARADFISVHLPKTPETA | |
| | | | GLIDKEALAKTKPGVIIVNAARGGLVDEAALADAITGGHVRA | |
| | | | AGLDVFATEPCTDSPLFELAQVVVTPHLGASTAEAQDRAGTD | |
| | | | VAESVRLALAGEFVPDAVNVGGGVVNEEVAPWLDLVRKLGVL | |
| | | | AGVLSDELPVSLSVQVRGELAAEEVEVLRLSALRGLFSAVIE | |
| | | | DAVTFVNAPALAAERGVTAEICKASESPNHRSVVDVRAVGAD | |
| | | | GSVVTVSGTLYGPQLSQKIVQINGRHFDLPAQGINLIIHYVD | |
| | | | RPGALGKIGTLLGTAGVNIQAAQLSEDAEGPGATILLRLDQD | |
| | | | VPDDVRTAIAAAVDAYKLEVVDLS | |
| serA | Mycobacterium | CAB16440 | MDLPVVLIADKLAQSTVAALGDQVEVRWVDGPDRTKLLAAVP | 89 |
| | leprae (use this | | EADALLVRSATTVDAEVLAAAPKLKIVAPAGVGLDNVDVDAA | |
| | to clone M. | | TARGVLVVNAPTSNIHSAAEHALALLLAASRQIAEADASLRA | |
| | smegmatis | | HIWKRSSFSGTEIFGKTVGVVGLGRIGQLVAARIAAFGAHVI | |
| | gene) | | AYDPYVAPARAAQLGIELMSFDDLLARADFISVHLPKTPETA | |
| | | | GLIDKEALAKTKPGVIIVNAARGGLVDEVALADAVRSGHVRA | |
| | | | AGLDVFATEPCTDSPLFELSQVVVTPHLGASTAEAQDRAGTD | |
| | | | VAESVRLALAGEFVPDAVNVDGGVVNEEVAPWLDLVCKLGVL | |
| | | | VAALSDELPASLSVHVRGELASEDVEILRLSALRGLFSTVIE | |
| | | | DAVTFVNAPALAAERGVSAEITTGSESPNHRSVVDVRAVASD | |
| | | | GSVVNIAGTLSGPQLVQKIVQVNGRNFDLRAQGMNLVIRYVD | |
| | | | QPGALGKIGTLLGAAGVNIQAAQLSEDTEGPGATILLRLDQD | |
| | | | VPGDVRSAIVAAVSANKLEVVNLS | |
| serA | Thermobifida | ZP_00057280 | MAATAVEPTRTPSKEFVVPKPVVLVAEELSPAGIALLEEDFE | 90 |
| | fusca | | VRHVNGADRSQLLPALAGVDALIVRSATKVDAEVLAAAPSLK | |
| | | | VVARAGVGLDNVDVEAATKAGVLVVNAPTSNIISAAEQAINL | |
| | | | LLATAPNTAAAHAALVRGEWKRSKYTGVELYDKTVGIVGLGR | |
| | | | IGVLVAQRLQAFGTKLIAYDPFVQPARAAQLGVELVELDELL | |
| | | | ERSDFITIHLPKTKDTIGLIGEEELRKVKPTVRIINAARGGI | |
| | | | VDETALYHALKEGRVAGAGLDVFAKEPCTDSPLFELENVVVA | |
| | | | PHLGASTHEAQEKAGTQVARSVKLALAGEFVPDAVNIQGKGV | |
| | | | SEDIKPGLPLTEKLGRILAALADGAITRVEVEVRGEIVAHDV | |
| | | | KVIELAALKGLFTDIVEEAVTYVNAPLVAKERGIEVSLTTEE | |
| | | | ESPDWRNVITVRAILSDGQRVSVSGTLTGPRQLEKLVEVNGY | |
| | | | TMEIAPSEHMAFFSYHDRPGVVGVVGQLLGQAQVNIAGMQVS | |
| | | | RDKEGGAALIALTVDSAIPDETLETISKEIGAEISRVDLVD | |
| serA | Streptomyces | CAB37591 | MSSKPVVLIAEELSPATVDALGPDFEIRHCNGADRAELLPAI | 91 |
| | coelicolor | | ADVDAILVRSATKVDAEAVAAAKKLKVVARAGVGLDNVDVSA | |
| | | | ATKAGVMVVNAPTSHIVTAAELACGLIVATARNIPQANAALK | |
| | | | NGEWKRSKYTGVELAEKTLGVVGLGRIGALVAQRMSAFGMKV | |
| | | | VAYDPYVQPAPAAQMGVKVLSLDELLEVSDFITVHLPKTPET | |
| | | | LGLIGDEALRKVKPSVRIVNAARGGIVDEEALYSALKEGRVA | |
| | | | GAGLDVYAKEPCTDSPLFEFDQVVATPHLGASTDEAQEKAGI | |
| | | | AVAKSVRLALAGELVPDAVNVQGGVIAEDVKPGLPLAERLGR | |
| | | | IFTALAGEVAVRLDVEVYGEITQHDVKVLELSALKGVFEDVV | |
| | | | DETVSYVNAPLFAQERGVEVRLTTSSESPEHRNVVIVRGTLS | |
| | | | DGEEVSVSGTLAGPKHLQKIVAIGEYDVDLALADHMVVLRYE | |
| | | | DRPGVVGTVGRIIGEAGLNIAGMQVARATVGGEALAVLTVDD | |
| | | | TVPSGVLAEVAAEIGATSARSVNLV | |
| serA | Lactobecilus | CAD63373 | MTKVFIAGQLPAQANTLLLQSQLVIDTYTGDNLISHAELIRR | 92 |
| | plantarum | | VADADFLIIPLSTQVDQDVLDHAPHLKLIANFGAGTNNIDIA | |
| | | | AAAKRQIPVTNTPNVSAVATAESTVGLIISLAHRIVEGDHLM | |
| | | | RTSGFNGWAPLFFLGHNLQGKTLGILGLGQIGQAVAKRLHAF | |
| | | | DMPILYSQHHRLPISRETQLGATFVSQDELLQRADIVTLHLP | |
| | | | LTTQTTHLIDNAAFSKMKSTALLINAARGPIVDEQALVTALQ | |
| | | | QHQIAGAALDVYEHEPQVTPGLATMNNVILTPHLGNATVEAR | |
| | | | DGMATIVAENVIAMAQHQPIKYVVNDVTPA | |
| serA | Coryne- | BAB98677 | MSQNGRPVVLIADKLAQSTVDALGDAVEVRWVDGPNRPELLD | 232 |
| | bacterium | | AVKEADALLVRSATTVDAEVIAAAPNLKIVGRAGVGLDNVDI | |
| | glutamicum | | PAATEAGVMVANAPTSNIHSACEHAISLLLSTARQIPAADAT | |
| | | | LREGEWKRSSFNGVEIFGKTVGIVGFGHIGQLFAQRLAAFET | |
| | | | TIVAYDPYANPAPAAQLNVELVELDELMSRSDFVTIHLPKTK | |
| | | | ETAGMFDAQLLAKSKKGQIIThAARGGLVDEQALADAIESGH | |
| | | | IRGAGFDVYSTEPCTDSPLFKLPQVVVTPHLGASTEEAQDRA | |
| | | | GTDVADSVLKALAGEFVADAVNVSGGRVGEEVAVWMDLARKL | |
| | | | GLLAGKLVDAAPVSIEVEARGELSSEQVDALGLSAVRGLFSG | |
| | | | IIEESVTFVNAPRIAEERGLDISVKThSESVTHRSVLQVKVI | |
| | | | TGSGASATVVGALTGLERVEKITRINGRGLDLRAEGLNLFLQ | |
| | | | YTDAPGALGTVGTKLGAAGINIEAAALTQAEKGDGAVLILRV | |
| | | | ESAVSEELEAEINAELGATSFQVDLD | |
| serA | Escherichia coli | NP_417388 | MAKVSLEKDKIKFLLVEGVHQKALESLRAAGYTNIEFHKGAL | 233 |
| | | | DDEQLKESIRDAHFIGLRSRTHLTEDVINAAEKLVAIGCFCI | |
| | | | GTNQVDLDAAAKRGIPVFNAPFSNTRSVAELVIGELLLLLRG | |
| | | | VPEANAKAHRGVWNKLAAGSFEARGKKIGIIGYGHIGTQLGI | |
| | | | LAESLGMYVYFYDIEMCLPLGNATQVQHLSDLLNMSDVVSLH | |
| | | | VPENPSTKNMMGAKEISLMKPGSLLINASRGTVVDIPALCDA | |
| | | | LASKHLAGAAIDVFPTEPATNSDPFTSPLCEFDNVLLTPHIG | |
| | | | GSTQEAQENIGLEVAGKLIKYSDNGSTLSAVNFPEVSLPLHG | |
| | | | GRRLMHIHENRPGVLTALNKIFAEQGVNIAAQYLQTSAQMGY | |
| | | | VVIDIEADEDVAEKALQANKAIPGTIRARLLY | |
| lysE | Mycobacterium | CAA98398 | MNSPLVVGFLACFTLIAAIGAQNAFVLRQGIQREHVLPVVAL | 93 |
| | tuberculosis (use | | CTVSDIVLIAAGIAGFGALIGAHPRALNVVKFGGAAFLIGYG | |
| | this to clone M. | | LLAARRAWRPVALIPSGATPVRLAEVLVTCAAFTFLNPHVYL | |
| | smegmatis | | DTVVLLGALANEHSDQRWLFGLGAVTASAVWFATLGFGAGRL | |
| | gene) | | RGLFTNPGSWRILDGLIAVMMVALGISLTVT | |
| lysE | Mycobacterium | CAB00949 | MMTLKVAIGPQNAFVLRQGIRREYVLVIVALCGIADGALIAA | 94 |
| | tuberculosis (use | | GVGGFAALIHAHPNMTLVARFGGAAFLIGYALLAARNAWRPS | |
| | this to clone M. | | GLVPSESGPAALIGVVQMCLVVTFLNPHVYLDTVVLIGALAN | |
| | smegmatis | | EESDLRWFFGAGAWAASVVWFAVLGFSAGRLQPFFATPAAWR | |
| | gene | | ILDALVAVTMIGVAVVVLVTSPSVPTANVALII | |
| lysE | Streptomyces | CAB93746 | MNNALTAAAAGFGTGLSLIVAIGAQNAFVLRQGVRRDAVLAV | 95 |
| | coelicolor | | VGICALSDAVLIALGVGGVGAVVVAWPGALTAVGWIGGAFLL | |
| | | | CYGALAARRVFRPSGALRADGAAAGSRRRAVLTCLALTWLNP | |
| | | | HVYLDTVFLLGSVAADRGPLRWTFGLGAAAASLVWFAALGFG | |
| | | | ARYLGRFLSRPVAWRVLDGLVAATMIVLGVSLVAGA | |
| lysE | Lactobacillus | CAD63877 | MQVFLQGLLFGIVYIAPIGMQNLFVVSTAIEQPLQRALRVAL | 96 |
| | plantarum | | IVIAFDTSLSLACFYGVGRLLQTTPWLELGVLLIGSLLVFYI | |
| | | | GWNLLRKKATAMGTLDADFSYKAAILTAFSVAWLNPQALIDG | |
| | | | SVLLAAFRVSIPAALTHFFMLGVILASIIWFIGLTSLISKFK | |
| | | | LMQPRVLLWINRICGGIIILYGVQLLATFITKI | |
| lysE | Coryne- | CAA65324 | MEIFITGLLLGASLLLSIGPQNVLVIKQGIKREGLIAVLLVC | 234 |
| | bacterium | | LISDVFLFIAGTLGVDLLSNAAPIVLDIMRWGGIAYLLWFAV | |
| | glutamicum | | MAAKDAMTNKVEAPQIIEETEPTVPDDTPLGGSAVATDTRNR | |
| | | | VRVEVSVDKQRVWVKPMLMAIVLTWLNPNAYLDAFVFIGGVG | |
| | | | AQYGDTGRWIFAAGAFAASLIWFPLVGFGAAALSRPLSSPKV | |
| | | | WRWINVVVAVVMTALAIKLMLMG | |
| metB | Mycobacterium | CAA17195 | MSEDRTGHQGISGPATRAIHAGYRPDPATGAVNVPIYASSTF | 97 |
| | tuberculosis (use | | AQDGVGGLRGGFEYARTGNPTRAALEASLAAVEEGAFAPAFS | |
| | this to clone M. | | SGMAATDCALRAMLRPGDHVVIPDDAYGGTFRLIDKVFTRWD | |
| | smegmatis | | VQYTPVRLADLDAVGAAITPRTRLIWVETPTNPLLSIADITA | |
| | gene) | | IAELGTDRSAKVLVDNTFASPALQQPLRLGADVVLHSTTKYI | |
| | | | GGHSDVVGGALVTNDEELDEEFAFLQNGAGAVPGPFDAYLTM | |
| | | | RGLKTLVLRMQRHSENACAVAEFLADHPSVSSVLYPGLPSHP | |
| | | | GHEIAARQMRGFGGMVSVRMRAGRRAAQDLCAKTRVFILAES | |
| | | | LGGVESLIEHPSAMTHASTAGSQLEVPDDLVRLSVGIEDIAD | |
| | | | LLGDLEQALG | |
| metB | Mycobacterium | AAA63036 | MSEDYRGHHGITGLATKAIHAGYRPDPATGAVNVPIYASSTF | 98 |
| | leprae (use this | | AQDGVGELRGGFEYARTGNPMRAALEASLATVEEGVFARAFS | |
| | to clone M. | | SGMAASDCALRVMLRPGDHVIIPDDVYGGTFRLIDKVFTQWN | |
| | smegmatis | | VDYTPVPLSDLDAVRAAITSRTRLIWVETPTNPLLSIADITS | |
| | gene) | | IGELGKKHSVKTLVDNTFASPALQQPLMLGALVVLHSTTKYI | |
| | | | GGHSDVVGGALVTNDEELDQAFGFLQNGAGAVPSPFDAYLTM | |
| | | | RGLKTLVLRMQRHNENAITVAEFLAGHPSVSAVLYPGLPSHP | |
| | | | GHEVAARQMRGFGGMVSLRMRAGRLAAQDLCARTKVFTLAES | |
| | | | LGGVESLIEQPSAMTHASTTGSQLEVPDDLVRLSVGIEDVGD | |
| | | | LLCDLKQALN | |
| metB | Streptomyces | CAD30944 | MPMSDRHISQHFETLAIHAGNTADPLTGAVVPPIYQVSTYKQ | 99 |
| | coelicolor | | DGVGGLRGGYEYSRSANPTRTALEENLAALEGGRRGLAFASG | |
| | | | LAAEDCLLRTLLRPGDHVVIPNDAYGGTFRLFAKVATRWGVE | |
| | | | WSVADTSDAAAVRAALTPKTKAVWVETPSNPLLGITDIAQVA | |
| | | | QVARDAGARLVVDNTFATPYLQQPLALGADVVVHSLTKYMGG | |
| | | | HSDVVGGALIVGDQELGEELAFHQNANGAVAGPFDSWLVLRG | |
| | | | TKTLAVRMDRHSENATKVADMLSRHARVTSVLYPGLPEHPGH | |
| | | | EVAAKQMKAFGGMVSFRVEGGEQAAVEVCNRAKVFTLGESLG | |
| | | | GVESLIEHPGRMTHASAAGSALEVPADLVRLSVGIENADDLL | |
| | | | ADLQQALG | |
| metB | Thermobifida | ZP_00059348 | MSYEGFETLAIHAGQEADAETGAVVVPIYQTSTYRQDGVGGL | 100 |
| | fusca | | RGGYEYSRTANPTRTALEECLAALEGGVRGLAFASGMAAEDT | |
| | | | LLRTIARPGDHLIIPNDAYGGTFRLVSKVFERWGVSWDAVDL | |
| | | | SNPEAVRTAIRPETVAIWVETPTNPLLNIADIAALADIAHAA | |
| | | | DALLVVDNTFASPYLQRPLSLGADVVVHSTTKYLGGHSDVVG | |
| | | | GALVVADAELGERLAFHQNSMGAVAGPFDAWLTLRGIKTLGV | |
| | | | RMDRHCANAERVVEALVGHPEVAEVLYPGLSDHPGHKVAVDQ | |
| | | | MRAFGGMVSFRMRGGEEAALRVCAKTKVFTLAESLGGVESLI | |
| | | | EHPGKMTHASTAGSLLEVPSDLVRLSVGIETVDDLVNDLLQA | |
| | | | LEP | |
| metB | Lactobacillus | CAD62912 | MKFETQLIHGGISEDATTGATSVPIYMASTFRQTKIGQNQYE | 101 |
| | plantarum | | YSRTGNPTRAAVEALIATLEHGSAGFAFASGSAAINTVFSLF | |
| | | | SAGDHIIVGNDVYGGTFRLIDAVLKHFGMTFTAVDTRDLAAV | |
| | | | EAAITPTTKAIYLETPTNPLLHITDIAAIAKLAQAHDLLSII | |
| | | | DNTFASPYVQKPLDLGVDIVLHSASKYLGGHSDVIGGLVVTK | |
| | | | TPALGEKIGYLQNAIGSILAPQESWLLQRGMKTLALRMQAHL | |
| | | | NNAAKIFTYLKSHPAVTKIYYPGDPDNPDFSIAKQQMNGFGA | |
| | | | MISFELQPGMNPQTFVEHLQVITLAESLGALESLIEIPALMT | |
| | | | HGAIPRTIRLQNGIKDELIRLSVGVEASDDLLADLERGFASI | |
| | | | QAD | |
| metB | Coryne- | AAD54070 | MSFDPNTQGFSTASIHAGYEPDDYYGSINTPIYASTTFAQNA | 235 |
| | bacterium | | PNELRKGYEYTRVGNPTIVALEQTVAALEGAKYGRAFSSGMA | |
| | glutamicum | | ATDILFRIILKPGDHIVLGNDAYGGTYRLIDTVFTAWGVEYT | |
| | | | VVDTSVVEEVKAAIKDNTKLIWVETPTNPALGITDIEAVAKL | |
| | | | TEGTNAKLVVDNTFASPYLQQPLKLGAHAVLHSTTKYIGGHS | |
| | | | DVVGGLVVTNDQEMDEELLFMQGGIGPIPSVFDAYLTARGLK | |
| | | | TLAVRMDRHCDNAEKIAEFLDSRPEVSTVLYPGLKNHPGHEV | |
| | | | AAKQMKRFGGMISVRFAGGEEAAKKFCTSTKLICLAESLGGV | |
| | | | ESLLEHPATMTHQSAAGSQLEVPRDLVRISIGIEDIEDLLAD | |
| | | | VEQALNNL | |
| metB | Escherichia coli | NP_418374 | MTRKQATIAVRSGLNDDEQYGCVVPPIHLSSTYNFTGFNEPR | 236 |
| | | | AHDYSRRGNPTRDVVQRALAELEGGAGAVLTNTGMSAIHLVT | |
| | | | TVFLKPGDLLVAPHDCYGGSYRLFDSLAKRGCYRVLFVDQGD | |
| | | | EQALRAALAEKPKNVLVESPSNPLLRVVDIAKICHLAREVGA | |
| | | | VSVVDNTFLSPALQNPLALGADLVLHSCTKYLNGHSDVVAGV | |
| | | | VIAKDPDVVTELAWWANNIGVTGGAFDSYLLLRGLRTLVPRM | |
| | | | ELAQRNAQAIVKYLQTQPLVKKLYHPSLPENQGHEIAARQQK | |
| | | | GFGANLSFELDGDEQTLRRFLGGLSLFTLAESLGGVESLISH | |
| | | | AATMTHAGMAPEAPAAAGISETLLRISTGIEDGEDLIADLEN | |
| | | | GFPAANKG | |
| putative | Streptomyces | CAB40862 | MAGIGAFWSVSFLLVLVPGADWAYAITAGLRHRSVLPAVGGM | 102 |
| threonine | coelicolor | | LSGYVLLTAVVAAGLATAVAGSPTVLTALTAAGAAYLIWLGA | |
| efflux | | | TTLARPAAPRAEEGDQGDGSGSLVGRAARGAGISGLNPKALL | |
| protein 1 | | | LFLALLPQFAARDADWPFAAQIVALGLVHTANCAVVYTGVGA | |
| | | | TARRILGARPAVATAVSRFSGAAMILVGALLLVERLLAQGPT | |
| threonine | Coryne- | NP_601855 | MDAASWVAFALALLVANAVPGPDLVLVLHSATRGIRTGVMTA | 196 |
| efflux | bacterium | | AGIMTGLMLHASLAIAGATALLLSAPGVLSAIQLLGAGVLLW | |
| protein | glutamicum | | MGTNMFRASQNTGESETAASQSSAGYFRGFITNATNPKALLF | |
| | | | FAAILPQFIGNGEDMKMRTLANCATIVLGSGAWWLGTIALVR | |
| | | | GIGLQKLPSADRIITLVGGIALFLIGAGLLVNTAYGLIT | |
| hypothetical | Streptomyces | CAB42763 | MSVPGSVAQVTEAEEPKPQSDEARSAFRQPSGIAASIDGESS | 103 |
| protein | coelicolor | | TTSEFEIPQGFAVPRHAGTESETTSEFSLPDGLEVPQAPPAD | |
| NCgl2533 | | | TEGSAFTMPSTHSAWTAPTAFTPASGFPAVSLTDVPWQDRMR | |
| related | | | AMLRMPVAERPAPEPSQKHDDETGPAVPRVLDLTLRIGELLL | |
| | | | AGGEGAEDVEAANFAVCRSYGLDRCEPNVTFTLLSISYQPSL | |
| | | | VEDPVTASRTVRRRGTDYTRLAAVFHLVDDLSDPDTNISLEE | |
| | | | AYRRLAEIRRNRHPYPTWVLTVASGLLAGGASLLVGGGLTVF | |
| | | | FAAMFGSMLGDRLAWLCAGRGLPEFYQFAVAAMPPAAMGVVL | |
| | | | TVTHVDVKASAVITGGLFALLPGRALVAGVQDGLTGFYITAA | |
| | | | ARLLEVMYFFVSIVAGVLVVLYFGVQLGAELHPDAKLGTGDE | |
| | | | PFVQIFASMLLSLAFAILLQQERATVLAVTLNGGIAWCVYGA | |
| | | | MNYAGDISPVASTAAAAGLVGLFGQLMSRYRFASALPYTTAA | |
| | | | IGPLLPGSATYFGLLGIAQGEVDSGLLSLSNAVALAMAIAIG | |
| | | | VNLGGEISRLFLKVPGAASAAGRRAAKRTRGF | |
| hypotheti- | Mycobacterium | AAK48209 | MDQDRSDNTALRRGLRIALRGRRDPLPVAGRRSRTSGGIGDL | 104 |
| cal | tuberculosis (use | | HTRKVLDLTIRLAEVMLSSGSGTADVVATAQDVAQAYQLTDC | |
| protein | this to clone M. | | VVDITVTTIIVSALATTDTPPVTIMRSVRTRSTDYSRLAELD | |
| NCgl2533 | smegmatis | | RLVQRITSGGVAVDQAHEANDELTERPHPYPRWLATAGAAGF | |
| related | gene) | | ALGVAMLLGGTWLTCVLAAVTSGVIDRLGRLLNRIGTPLFFQ | |
| | | | RVFGAGIATLVAVAAYLIAGQDPTALVATGIVVLLSGMTLVG | |
| | | | SMQDAVTGYMLTALARLGDALFLTAGIVVGILISLRGVTNAG | |
| | | | IQIELHVDATTTLATPGMPLPILVAVSGAALSGVCLTIASYA | |
| | | | PLRSVATAGLSAGLAELVLIGLGAAGFGRVVATWTAAIGVGF | |
| | | | LATLISIRRQAPALVTATAGIMPMLPGLAVFRAVFAFAVNDT | |
| | | | PDGGLTQLLEAAATALALGSGVVLGEFLASPLRYGAGRIGDL | |
| | | | FRIEGPPGLRRAVGRVVRLQPAKSQQPTGTGGQRWRSVALEP | |
| | | | TTADDVDAGYRGDWPATCTSATEVR | |
| hypotheti- | Mycobacterium | CAA18059 | MDQDRSDNTALRRGLRIALRGRRDPLPVAGRRSRTSGGIDDL | 105 |
| cal | tuberculosis (use | | HTRKVLDLTIRLAEVMLSSGSGTADVVATAQDVAQAYQLTDC | |
| protein | this to clone M. | | VVDITVTTIIVSALATTDTPPVTIMRSVRTRSTDYSRLAELD | |
| NCgl2533 | smegmatis | | RLVQRITSGGVAVDQAHEAMDELTERPHPYPRWLATAGAAGF | |
| related | gene) | | ALGVAMLLGGTWLTCVLAAVTSGVIDRLGRLLNRIGTPLFFQ | |
| | | | RVFGAGIATLVAVAAYLIAGQDPTALVATGIVVLLSGMTLVG | |
| | | | SMQDAVTGYMLTALARLGDALFLTAGIVVGILISLRGVTNAG | |
| | | | IQIELHVDATTTLATPGMPLPILVAVSGAALSGVCLTIASYA | |
| | | | PLRSVATAGLSAGLAELVLIGLGAAGFGRVVATWTAAIGVGF | |
| | | | LATLISIRRQAPALVTATAGIMPMLPGLAVFRAVFAFAVNDT | |
| | | | PDGGLTQLLEAAATALALGSGVVLGEFLASPLRYGAGRIGDL | |
| | | | FRIEGPPGLRRAVGRVVRLQPAKSQQPTGTGGQRWRSVALEP | |
| | | | TTADDVDAGYRGDWPATCTSATEVR | |
| hypotheti- | Thermobifida | ZP_000595 | MISYGPVADRCRVGATSAAWGTSPPMSFPFLPLVSHPLPYVP | 106 |
| cal | fusca | | GLDASFPDGACVPLGRGPSRGGERRMNQAPRRSDTSHSPTLL | |
| protein | | | TRLRDWRASRGVLDLEAEEFEDEAPRPDPRAMDLVLRVGELL | |
| NCgl2533 | | | LASGEATETVSDAMLSLAVAFELPRSEVSVTFTGITLSCHPG | |
| related | | | GDEPPVTGERVVRRRSLDYHKVNELHALVEDAALGLLDVERA | |
| | | | TARLHAIKRSRPHYPRWVIVAGLGLIASSASVMVGGGIIVAA | |
| | | | TAFAATVLGDRAAGWLARRGVAEFYQMAVAALLAASTGMALL | |
| | | | WVSEELELGLRAMAVITGSIVALLPGRPLVSSLQDGISGAYV | |
| | | | SAAARLLEVFFMLGAIVAGVGAVAYTAVRLGLYVDLDNLPSA | |
| | | | GTSLEPVVLAAAAGLALAFAVSLVAPVRALLPIGANGVLIWV | |
| | | | CYAGLRELLAVPPVVGTGAGAVVVGVIGHWLARRTRRPPLTF | |
| | | | IIPSIAPLLPGSILYRGLIEMSTGEPLAGVASLGEAVAVGLA | |
| | | | LGAGVNLGGELVPAFSWGGLVGAGRRGRQAARRTRGGY | |
| hypotheti- | Lactobacillus | CAD62758 | MNKERKSVMPLSQRHHMTIPWKDFIRNEDVPAKHASLQERTS | 107 |
| cal | plantarum | | IVGRVGILMLSCGTGAWRVRDAMNKIARSLNLTCSADIGLIS | |
| protein | | | IQYTCFHHERSYTQVLSIPNTGVNTDKLNILEQFVKDFDAKY | |
| NCgl2533 | | | ARLTVAQVHAAIDEVQTRPKQYSPLVLGLAAGLACSGFIFLL | |
| related | | | GGGIPEMICSFLGAGLGNYVRALMGKRSMTTVAGIAVSVAVA | |
| | | | CLAYMVSFKIFEYNFQILAQHEAGYIGAMLFVIPGFPFITSM | |
| | | | LDISKLDMRSGLERLAYAIMVTLIATLVGWLVATLVSFKPAL | |
| | | | FLPLGLSPLAVLLLRLPASFCGVYGFSIMFNSSQKMAITAGF | |
| | | | IGAIANTLRLELVDLTAMPPAAAAFCGALVAGLIASVVNRYN | |
| | | | GYPRISLTVPSIVIMVPGLYIYRAIYSIGNNQIGVGSLWLTK | |
| | | | AVLIIMFLPLGLFVAPALLDHEWRHFD | |
| NCgl2533 | Coryne- | NP_601823 | MLSFATLRGRISTVDAAKAAPPPSPLAPIDLTDHSQVAGVMN | 198 |
| | bacterium | | LAARIGDILLSSGTSNSDTKVQVRAVTSAYGLYYTHVDITLN | |
| | glutamicum | | TITIFTNIGVERKMPVNVFHVVGKLDTNFSKLSEVDRLIRSI | |
| | | | QAGATPPEVAEKILDELEQSPASYGFPVALLGWAMMGGAVAV | |
| | | | LLGGGWQVSLIAFITAFTIIATTSFLGKKGLPTFFQNVTGGF | |
| | | | IATLPASIAYSLALQFGLEIKPSQIIASGIVVLLAGLTLVQS | |
| | | | LQDGITGAPVTASARFFETLLFTGGIVAGVGLGIQLSEILHV | |
| | | | MLPAMESAAAPNYSSTFARIIAGGVTAAAFAVGCYAEWSSVI | |
| | | | IAGLTALMGSAFYYLFVVYLGPVSAAAIAATAVGFTGGLLAR | |
| | | | RFLIPPLIVAIAGITPMLPGLAIYRGMYATLNDQTLMGFTNI | |
| | | | AVALATASSLAAGVVLGEWIARRLRRPPRFNPYRAFTKANEF | |
| | | | SFQEEAEQNQRRQRKRPKTNQRFGNKR | |
| putative | Thermobifida | ZP_000569 | MSGGVMADITRNRSSGLAFAIASALAFGGSGPVARPLIDAGL | 108 |
| membrane | fusca | | DPLHVTWLRVAGAALLLLPVAFRHHRTLRTRPALLLAYGVFP | |
| protein | | | MAGVQAFYFAAISRIPVGVALLIEFLGPVLVLLWTRLVRRIP | |
| NCgl0580 | | | VSRAASLGVALAVIGLGCLVEVWAGIRLDAVGLILALAAAVC | |
| related | | | QATYFLLSDTARDDVDPLAVISYGALIATALLSLLARPWTLP | |
| | | | WGILAQNVGFGGLDIPALILLVWLALVATTIAYLTGVAAVRR | |
| | | | LSPVVAGGVAYLEVVTSIVLAWLLLGEALSVAQLVGAAAVVT | |
| | | | GAFLAQTAVPDTSAAQGPETLPTAQDPAPQTGSAR | |
| putative | Thermobifida | ZP_000594 | MNSDSPGQSAPGPFSRAAALVRAAGTAIPATWLVGVSILSVQ | 109 |
| membrane | fusca | | FGAGVAKNLFAVLPPSTVVWLRLLASALVLLCFAPPPLRGHS | |
| protein | | | RTDWLVAVGFGTSLAVMNYAIYESFARIPLGVAVTIEFLGPL | |
| NCgl0580 | | | AVAVAGSRRWRDLVWVVLAGTGVALLGWDDGGVTLAGVAFAA | |
| related | | | LAGAAWACYILLSAATGRRFPGTSGLTVASVIGAVLVAPMGL | |
| | | | AHSSPALLDPSVLLTGLAVGLLSSVIPYSLEMQALRRIPPGV | |
| | | | FGILMSLEPAAAALVGLVLLGEFLTVAQWAAVACVVVASVGA | |
| | | | TRSARL | |
| putative | Thermobifida | ZP_000580 | MWTLDLPLKRNDSSTNGAWTETENRRHSGGMILSFVSLVRHA | 110 |
| membrane | fusca | | HLRVPAPLLTVLSLVLLHMGSAGAVHLFAIAGPLEVTWLRLS | |
| protein | | | WAALLLFAVGGRPLLRAARAATWSDLAATAALGVVSAGMTLL | |
| NCgl0580 | | | FSLALDRIPLGTAAAIEFLGPLTVSVLALRRRRDLLWIVLAV | |
| related | | | AGVLLLTRPWHGEANLLGIAFGLGGAVCVALYIVFSQTVGSR | |
| | | | LGVLPGLTLANTVSALVTAPLGLPGAMAAADRHLVAATLGLA | |
| | | | LIYPLLPLLLEMVSLQRMNRGTFGILVSVDPAIGLLIGLLLI | |
| | | | GQVPVPLQVAGMALVVAAGLGATRGTSGRTRGGADPHATDGE | |
| | | | PEDRTPDRPAPDDAGHHTTDPVTV | |
| putative | Streptomyces | CAB71821 | MAATRPAVIALTALAPVSWGSTYAVTTEFLPPDRPLFTGLMR | 111 |
| membrane | coelicolor | | ALPAGLLLLALARVLPRGAWWGKAAVLGVLNIGAFFPLLFLA | |
| protein | | | AYRMPGGMAAVVGSVGPLLVVGLSALLLGQRPTTRSVLTGVA | |
| NCgl0580 | | | AASGVSLVVLEAAGALDPLGVLAALAATASMSTGTVLAGRWG | |
| related | | | RPEGVGPLALTGWQLTAGGLLLAPLALLVEGAPPALDGPAVG | |
| | | | GYLYLALANTALAYWLWFRGIGRLSATQVTFLGPLSPLTAAV | |
| | | | IGWAALGEALGPVQLAGTALAFGATLVGQTVPSAPRTPPVAA | |
| | | | GAGPFSSASRNGRKDSMDLTGAALRR | |
| putative | Streptomyces | CAB95885 | MPDGAPGGRFGALGPVGLVLAGGISVQFGAALAVSLMPRAGA | 112 |
| membrane | coelicolor | | LGVVTLRLAVAAVVMLLVCRPRLRGHSRADWGTVVVFGIAMA | |
| protein | | | GMNGLFYQAVDRIPLGPAVTLEVLGPLALSVFASRRAMNLVW | |
| NCgl0580 | | | AALALAGVFLLGGGGFDGLDPAGAAFALAAGAMWAAYIVFSA | |
| related | | | RTGRRFPQADGLALAMAVGALLFLPLGIVESGSKLIDPVTLT | |
| | | | LGAGVALLSSVLPYTLELLALRRLPAPTFAILMSLEPAIAAA | |
| | | | AGFLILDQALTATQSAAIALVIAASMGAVRTQVGRRRAKALP | |
| putative | Streptomyces | CAB46802 | MMTTARTSPPAPWHRRPDLLAAGAATVTVVLWASAFVSIRSA | 113 |
| membrane | coelicolor | | GEAYSPGALALGRLLSGVLTLGAIWLLRREGLPPRAAWRGIA | |
| protein | | | ISGLLWFGFYMVVLNWGEQQVDAGTAALVVNVGPILIALLGA | |
| NCgl0580 | | | RLLGDALPPRLLTGMAVSFAGAVTVGLSMSGEGGSSLFGVVL | |
| related | | | CLLAAVAYAGGVVAQKPALAHASALQVTTFGCLVGAVLCLPF | |
| | | | AGQLVHEAAGAPVSATLNMVYLGVFPTALAFTTWAYALARTT | |
| | | | AGRMGATTYAVPALVVLMSWLALGEVPGLLTLAGGALCLAGV | |
| | | | AVSRSRRRPAAVPDRAAPTAEPRREDAGRA | |
| putative | Streptomyces | CAC32287 | MPVHTSDSARGSRGKGIGLGLALASAVAFGGSGVAAKPLIEA | 114 |
| membrane | coelicolor | | GLDPLHVVWLRVAGAALVMLPLAVRHPALPRRRPALVAGYGL | |
| protein | | | FAVAGVQACYFAAISRIPVGVALLVEYLAPALVLGWVRFVQR | |
| NCgl0580 | | | RPVTRAAALGVVLAVGGLACVVEVWSGLGFDALGLLLALGAA | |
| related | | | CCQVGYFVLSDQGSDAGEEAPDPLGVIAYGLLVGAAVLTIVA | |
| | | | RPWSMDWSVLAGSAPMDGTPVAAALLLAWIVLIATVLAYVTG | |
| | | | IVAVRRLSPQVAGVVACLEAVIATVLAWVLLGEHLSAPQVVG | |
| | | | GIVVLAGAFIAQSSTPAKGSADPVARGGPERELSSRGTST | |
| putative | Erwinia | S35974 | MKLKDFAFYAPCVWGTTYFVTTQFLPADKPLLAALIRALPAG | 115 |
| membrane | chrysanthemi | | IILILGKNLPPVGWLWRLFVLGALNIGVFFVMLFFAAYRLPG | |
| protein | | | GVVALVGSLQPLIVILLSFLLLTQPVLKKQMVAAVAGGIGIV | |
| NCgl0580 | | | LLISLPKAPLNPAGLVASALATMSMASGLVLTKKWGRPAGMT | |
| related | | | MLTFTGWQLFCGGLVILPVQMLTEPLPDLVTLTNLAGYLYLA | |
| | | | IPGSLLAYFMWFSGLEANSPVIMSLLGFLSPLVALLLGFLFL | |
| | | | QQGLSGAQLVGVVFIFSALIIVQDISLFSRRKKVKPLEQSDC | |
| | | | AVK | |
| putative | regulatory | AAF74778 | MKLKDFAFYAPCVWGTTYFVTTQFLPADKPLLAALIRALPAG | 116 |
| membrane | protein PecM | | IILILGKTLPPVGWLWRLFVLGALNIGVFFVMLFFAAYRLPG | |
| protein | [Pecto-bacterium | | GVVALVGSLQPLIVILLSFLLLTQPVLKKQMVAAVAGGIGIA | |
| NCgl0580 | chrysanthemi] | | LLISLPKAPLNPAGLVASALATVSMASGLVLTKKWGRPAGMT | |
| related | | | MLTFTGWQLFCGGLVILPVQMLTEPLPDVVTLTNLAGYFYLA | |
| | | | IPGSLLAYFMWFSGIEANSPVMMSMLGFLSPLVALFLGFLFL | |
| | | | QQGLSGAQLVGVVFIFSAIIIVQDVSLFSRRKKVKQLEQSDC | |
| | | | AVK | |
| putative | Lactobacillus | CA063826 | MKRLVGTLCGIISAALFGLGGILAQPLLSEQVLTPQQIVLLR | 117 |
| membrane | plantarum | | LLIGGAMLLLYRNLFFKQARKSTKKIWTHWRILTRIMIYGIA | |
| protein | | | GLCTAQIAFFSAINYSNAAVATVFQSTSPFILLVFTALKAKR | |
| NCgl0580 | | | LPSLLAGMSLISALMGIWLIVESGFKTGLIKPEAIIFGLIAA | |
| related | | | IGVILYTKLPVPLLNQIAAVDILGWALVIGGVIALIHTPLPN | |
| | | | LVRFSKTQLLAVLIIVILATVVAYDLYLESLKLIDGFLATMT | |
| | | | GLFEPISSVLFGMLFLHQILVPQALVGIILNVGAIMILNLPH | |
| | | | HITAPVPSKTCQCTMSNQ | |
| putative | Lactobacillus | CAD62768 | MKKIAPLFVGLGAISFGIPASLFKIARRQGVVNGPLLFWSFL | 118 |
| membrane | plantarum | | SAVVILGVIQILRPARLRNQQTNWKQIGLVIAAGTASGFTNT | |
| protein | | | FYIQALKLIPVAVAAVMLMQAVWISTLLGAVIHHRRPSRLQV | |
| NCgl0580 | | | VSIVLVLIGTILAAGLFPITQALSPWGLMLSFLAACSYACTM | |
| related | | | QFTASLGNNLDPLSKTWLLCLGAFILIAIVWSPQLVTAPTTP | |
| | | | ATVGWGVLIALFSMVFPLVMYSLFMPYLELGIGPILSSLELP | |
| | | | ASIVVAFVLLDETIDWVQMVGVAIIITAVILPNVLNMRRVRP | |
| putative | Lactobacillus | CAD65468 | MTTNRYMKGIMWAMLASTLWGVSGTVMQFVSQNQAIPADWFL | 119 |
| membrane | plantarum | | SVRTLSAGIILLAIGFVQQGTKIFKVFRSWASVGQLVAYATV | |
| protein | | | GLMANMYTFYISIERGTAAAATILQYLSPLFIVLGTLLFKRE | |
| NCgl0580 | | | LPLRTDLIAFAVSLLGVFLAITKGNIHELAIPMDALVWGILS | |
| related | | | GVTAALYVVLPRKIVAENSPVVILGWGTLIAGILFNLYHPIW | |
| | | | IGAPKITPTLVTSIGAIVLIGTIFAFLSLLHSLQYAPSAVVS | |
| | | | IVDAVQPVVTFVLSIIFLGLQVTWVEILGSLLVIVAIYILQQ | |
| | | | YRSDPASD | |
| NCgl0580 | Coryne- | NP_599841 | MNKQSAAVLMVMGSALSLQFGAAIGTQLFPLNGPWAVTSLRL | 201 |
| | bacterium | | FIAGLIMCLVIRPRLRSWTKKQWIAVLLLGLSLGGMNSLFYA | |
| | glutamicum | | SIELIPLGTAVTIEFLGPLIFSAVLARTLKNGLCVALAFLGM | |
| | | | ALLGIDSLSGETLDPLGVIFAAVAGIFWVCYILASKKIGQLI | |
| | | | PGTSGLAVALITGAVAVFPLGATHMGPIFQTPTLLILALGTA | |
| | | | LLGSLIPYSLELSALRRLPAPIFSILLSLEPAFAAAVGWILL | |
| | | | DQTPTALKWAAIILVIAASIGVTWEPKKMLVDAPLHSKCNAK | |
| | | | RRVHTPS | |
| drug | Streptomyces | CAC32286 | MSNAVSGLPVGRGLLYLIVAGVAWGTAGAAASLVYPASDLGP | 120 |
| permease | coelicolor | | VALSFWRCANGLVLLLAVRPLRPRLRPRLRPRLRPAVREPFA | |
| NCgl2065 | | | RRTLRAGVTGVGLAVFQTAYFAAVQSTGLAVATVVTLGAGPV | |
| related | | | LIALGARLALGEQLGAGGAAAVAGALAGLLVLVLGGGSATVR | |
| | | | LPGVLLALLSAAGYSVMTLLTRWWGRGGGADAAGTSVGAFAV | |
| | | | TSLCLLPFALAEGLVPHTAEPVRLLWLLAYVAAVPTALAYGL | |
| | | | YFAGAAVVRSATVSVIMLLEPVSAAALAVLLLGEHLTAATLA | |
| | | | GTLLMLGSVAGLAVAETRAAREARTRPAPA | |
| drug | Streptomyces | CAA19979 | MNVLLSAAFVLCWSSGFIGAKLGAQTAATPTLLMWRFLPLAV | 121 |
| permease | coelicolor | | ALVAAAAVSRAAWRGLTPRDAGRQTAIGALSQSGYLLSVYYA | |
| NCgl2065 | | | IELGVSSGTTALIDGVQPLVAGALAGPLLRQYVSRGQWLGLW | |
| related | | | LGLSGVATVTVADAGAAGAEVAWWAYLVPFLGMLSLVAATFL | |
| | | | EGRTRVPVAPRVALTIHCATSAVLFSGLALGLGAAAPPAGSS | |
| | | | FWLATAWLVVLPTFGGYGLYWLILRRSGITEVNTLMFLMAPV | |
| | | | TAVWGALMFGEPFGVQTALGLAVGLAAVVVVRRGGGARRERP | |
| | | | VRSGADRPAAGGPTADQPTNRPTDRPTAAGSTDRPTADRR | |
| drug | Thermobifida | ZP_000581 | MSDFRKGVLYGASSYFMWGFLPLYWPLLTPPATAFEVLLHRM | 122 |
| permease | fusca | | IWSLVVTLVVLLVQRNWQWIRGVLRSPRRLLLLLASAALISL | |
| NCgl2065 | | | NWGAFITAVTTGHTLQSALAYFINPLVSVALGLLVFKERLRP | |
| related | | | GQWAALLLGVLAVAVLTVDYGSLPWLALAMAFSFAVYGALKK | |
| | | | FVGLDGVESLSAETAVLFLPALGGAVYLEVTGTGTFTSVSPL | |
| | | | HALLLVGAGVVTAAPLMLFGAAAHRIPLTLVGLLQFMVPVMH | |
| | | | FLIAWLVFGEDLSLGRWIGFAVVWTALVVFVVDMLRHARHTP | |
| | | | RPAPSAPVAEEAEETAAS | |
| drug | Streptomyces | CAC08293 | MAGSSRSDQRVGLLNGFAAYGMWGLVPLFWPLLKPAGAGETL | 123 |
| permease | coelicolor | | AHRMVWSLAFVAVALLFVRRWAWAGELLRQPRRLALVAVAAA | |
| NCgl2065 | | | VITVNWGVYIWAVNSGHVVEASLGYFINPLVTIAMGVLLLKE | |
| related | | | RLRPAQWAAVGTGFAAVLVLAVGYGQPPWISLCLAFSFATYG | |
| | | | LVKKKVNLGGVESLAAETAIQFLPALGYLLWLGAQGESTFTT | |
| | | | EGAGHSALLAATGVVTAIPLVCFGAAAIRVPLSTLGLLQYLA | |
| | | | PVFQFLLGVLYFGEAMPPERWAGFGLVWLALTLLTWDALRTA | |
| | | | RRTAPALREQLDRSGAGVPPLKGAAAAREPRVVASGTPAPGA | |
| | | | GDAPQQQQQQQQQQQQQQHGTRAGKP | |
| drug | Lactobacillus | CAD63209 | MKKAYLYIAISTLMFSSMEIALKMAGSAFNPIQLNLIRFFIG | 124 |
| permease | plantarum | | AIVLLPFALRALKQTGRKLVSADWRLFALTGLVCVIVSMSLY | |
| NCg12065 | | | QLAITVDQASTVAVLFSCNPVFALLFSYLILRERLGRANLIS | |
| related | | | VVISVIGLLIIVNPAHLTNGLGLLLAIGSAVTFGLYSIISRY | |
| | | | GSVKRGLNGLTMTCFTFFAGAFELLVLAWITKIPAVANGLTA | |
| | | | IGLRQFAAIPVLVNVNLNYFWLLFFIGVCVTGGGFAFYFLAM | |
| | | | EQTDVSTASLVFFIKPGLAPILAALILHEQILWTTVVGIVVT | |
| | | | LIGSVVTFVGNRFRERDTMGAIEQPTAAATDDEHVIKAAHAV | |
| | | | SNQEN | |
| NCgl2065 | Coryne- | NP_601347 | MNDAGLKTRNPVLAPILMVVNGVSLYAGAALAVGLFESFPPA | 199 |
| | bacterium | | LVAWMRVAAAAVILLVLYRPAVRNFIGQTGFYAAVYGVSTLA | |
| | glutamicum | | MNITFYEAIARIPMGTAVAIEFLGPIAVAALGSKTLRDWAAL | |
| | | | VLAGIGVIIISGAQWSANSVGVMFALAAALLWAAYIIAGNRI | |
| | | | AGDASSSRTGMAVGFTWASVLSLPLAIWWWPGLGATELTLIE | |
| | | | VIGLALGLGVLSAVIPYGLDQIVLRMAGRSYFALLLAILPIS | |
| | | | AALMGALALGQMLSVAELVGIVLVVIAVALRRPS | |
| predicted | 19553330 | NP_601332.1 | MIFGVLAYLGWGMFPAFFPLLLPAGPFEILAHRILWTAVLMM | 200 |
| permease | | | IIISFTSGWKELKSADRGTWLRIILSSLFIAGNWLIYVIAVN | |
| | | | SGQVTEAALGYFINPLLSVVLGIVFFKEQLRKLQISAVVIAA | |
| | | | AGVLVLTFLGDKPPYLAITLAFTFGIYGALKKQVKMSAASSL | |
| | | | CAETLVLLPIAVIYLIGLEASGHSTFFNNGSGHMALLICSGL | |
| | | | VTAVPLLMFALAAKAIPLSTVGMLQYLTPTMQMLWALFVVNE | |
| | | | SVEPMRWFGFVFIWIAVTIYITDSLLKK | |
| hypotheti- | Thermobifida | P_000582 | MNADTLLWSLLLGVIVVAAAAAIIIPTVRNSSTAPPPGAVGT | 125 |
| cal | fusca | | ALGAALTAAALGIAGSGTAPASEVPAGSGQVRTVDVVLGDMT | |
| membrane | | | VSPSHVTVAPGDSLVLRVRNEDTQVHDLVVETGARTPRLAPG | |
| protein | | | DSATLQVGTVTEPIDAWCTVLGHSAAGMRMRIDTTDTADSAD | |
| NCgl2829 | | | SPDTPAGADSGPPAPLPLSAEMSDDWQPRDAVLPPAPDRTEH | |
| related | | | EVEIRVTETELEVAPGVRQSVWTFGGDVPGPVLRGKVGDVFT | |
| | | | VTFVNDGTMGHGIDFHASSLAPDEPMRTINPGERLTYRFRAE | |
| | | | KAGAWVYHCSTSPMLQHIGNGMYGAVIIDPPDLEPVDREYLL | |
| | | | VQGELYLGEPGSADQVARMRAGEPDAWVFNGVAAGYAHAPLT | |
| | | | AEVGERVRIWVVAAGPTSGTSFHIVGAQFDTVYKEGAYLVRR | |
| | | | GDAGGAQALDLAVAQGGFVETVFPEAGSYPFVDHDMRHAENG | |
| | | | ARGFFTITE | |
| NCgl2829 | Coryne- | NP_602117 | MVLVIAGIIHPLLPEYRWVLIHLFTLGAITNSIVVWSQHFTE | 197 |
| | bacterium | | KFLHLKLEESKRPAQLLKIRVLNVGIIVTIIGQMIGQWIVTS | |
| | glutamicum | | VGATIVGGALAWHAGSLASQFRSAKRGQPFASAVIAYVASAC | |
| | | | CLPFGAFAGALLSKELSGHLQERVLLTHTVINFLGFVGFAAL | |
| | | | GSLSVLFAAIWRTKIRHNFTPWSVGIMAVSLPIIVTGILLNN | |
| | | | GYVAATGLAAYVAAWLLAMVGWGKASISNLSFSTSTSTTAPL | |
| | | | WLVGTLVWLAVQAVMHDGELYHVEVPTIALVIGFGAQLLIGV | |
| | | | MSYLLPSTMGGGASAVRTGTHILNTAGLFRWTLINGGLAIWL | |
| | | | LTDNSWLRVVVSLLSIGALAVFVILLPKAVRAQRGVITKKRE | |
| | | | PITPPEEPRLNQITAGISVLALILAAFGGLNPGVAPVASSNE | |
| | | | DVYAVTITAGDMVFIPDVIEVPAGKSLEVTMLNEDDMVHDLK | |
| | | | FANGVQTGRVAPGDEITVTVGDISEDMDGWCTIAGHRAQGMD | |
| | | | LEVKVAAPN | |
| yggA | Escherichia coli | AAA69090 | MFSYYFQGLALGAAMILPLGPQNAFVMNQGIRRQYHIMIALL | 237 |
| | | | CAISDLVLICAGIFGGSALLMQSPWLLALVTWGGVAFLLWYG | |
| | | | FGAFKTANSSNIELASAEVMKQGRWKIIATMLAVTWLNPHVY | |
| | | | LDTFVVLGSLGGQLDVEPKRWFALGTISASFLWFFGLALLAA | |
| | | | WLAPRLRTAKAQRIThLVVGCVMWFIALQLARDGIAHAQ | |
| | | | ALFS | |
| McbR | C. glutamicum | | MAASASGKSKTSAGANRRRNRPSPRQRLLDSATNLFTTEGIR | 363 |
| | | | VIGIDRILREADVAKASLYSLFGSKDALVIAYLENLDQLWRE | |
| | | | AWRERTVGMKDPEDKIIAFFDQCIEEEPEKDFRGSHFQNAAS | |
| | | | EYPRPETDSEKGIVAAVLEHREWCHKTLTDLLTEKNGYPGTT | |
| | | | QANQLLVFLDGGLAGSRLVHNISPLETARDLARQLLSAPPAD | |
| | | | YSI | |
| ThrB | C. glutamicum | NP_600410.1 | MAIELNVGRKVTVTVPGSSANLGPGFDTLGLALSVYDTVEVE | 364 |
| | | | IIPSGLEVEVFGEGQGEVPLDGSHLVVKAIRAGLKAADAEVP | |
| | | | GLRVVCHNNIPQSRGLGSSAAAAVAGVAAANGLADFPLTQEQ | |
| | | | IVQLSSAFEGHPDNAAASVLGGAVVSWTNLSIDGKSQPQYAA | |
| | | | VPLEVQDNIRATALVPNFHASTEAVRRVLPTEVTHIDARFNV | |
| | | | SRVAVMIVALQQRPDLLWEGTRDRLHQPYRAEVLPITSEW | |
| | | | VNRLRNRGYAAYLSGAGPTAMVLSTEPIPDKVLEDARESGIK | |
| | | | VLELEVAGPVKVEVNQP | |

TABLE 17

Nucleotide sequences of exemplary heterologous proteins for amino acid production in
Escherichia coli and coryneform bacteria. Note: This table provides coding sequences of each
gene. Some GenBank ® entries contain additional non-coding sequence associated with the gene.

| | | | | |
|---|---|---|---|---|
| | | GenBank ® | | SEQ ID |
| Gene | Organism | Nucleotide ID | NUCLEOTIDE SEQUENCE (CODING) | NO: |

| | | | | | |
|---|---|---|---|---|---|
| lysC | Mycobacterium | Z17372 | GTGGCGCTCGTCGTACAGAAATACGGCGGATCCTCGGT | 11 | |
| | smegmatis | | GGCGGACGCCGAGAGGATCCGACGGGTCGCCGAGCGGA | | |
| | | | TCGTCGAGACCAAGAAGGCGGGCAACGACGTCGTCGTC | | |
| | | | GTCGTCTCCGCGATGGGTGACACCACCGATGACCTGCT | | |
| | | | GGACCTGGCGCGCCAGGTGTCGCCCGCGCCGCCGCCGC | | |
| | | | GCGAGATGGACATGCTGCTGACCGCCGGTGAGCGGATC | | |
| | | | TCCAACGCGCTGGTCGCGATGGCCATCGAATCGCTCGG | | |
| | | | CGCGCAGGCCCGGTCCTTCACCGGATCGCAGGCCGGTG | | |
| | | | TGATCACCACGGGCACGCACGGCAACGCCAAGATCATC | | |
| | | | GACGTCACCCCGGGCCGGTTGCGCGACGCGCTCGACGA | | |
| | | | GGGGCAGATCGTGCTGGTCGCCGGGTTCCAGGGCGTCA | | |
| | | | GCCAGGACAGCAAGGACGTCACCACGCTGGGACGCGGC | | |
| | | | GGTTCGGACACCACGGCCGTCGCCGTGGCTGCGGCACT | | |
| | | | CGATGCCGATGTCTGCGAGATCTACACCGACGTCGACG | | |
| | | | GCATCTTCACCGCGGACCCGCGCATCGTGCCCAACGCC | | |
| | | | CGCCACCTCGACACCGTCTCCTTCGAGGAGATGCTGGA | | |
| | | | GATGGCGGCCTGCGGCGCGAAAGTTCTGATGCTGCGCT | | |
| | | | GCGTCGAGTACGCCCGCCGCTACAACGTGCCCATCCAC | | |
| | | | GTCCGGTCGTCGTATTCGGACAAGCCCGGCACCATCGT | | |
| | | | CAAAGGATCGATCGAGGACATCCCCATGGAAGACGCCA | | |
| | | | TCCTGACCGGAGTAGCCCACGACCGCAGCGAGGCCAAG | | |
| | | | GTCACGGTGGTCGGTCTGCCCGACGTTCCCGGCTACGC | | |
| | | | CGCCAAGGTGTTCCGCGCGGTCGCCGAGGCCGACGTGA | | |
| | | | ACATCGACATGGTGCTGCAGAACATCTCGAAGATCGAG | | |
| | | | GACGGCAAGACCGACATCACGTTCACGTGTGCGCGTGA | | |
| | | | CAACGGCCCGCGGGCCGTAGAGAAGCTCTCGGCGCTCA | | |
| | | | AGAGCGAGATCGGTTTCAGCCAGGTGCTGTACGACGAC | | |
| | | | CACATCGGCAAGGTGTCGCTGATCGGCGCCGGTATGCG | | |
| | | | GTCGCATCCGGGCGTGACGGCCACGTTCTGCGAGGCGC | | |
| | | | TCGCGGAGGCCGGCATCAACATCGACCTGATCTCGACG | | |
| | | | TCGGAGATCCGTATCTCGGTGCTCATCAAGGACACCGA | | |
| | | | ACTGGACAAGGCGGTTTCGGCGCTGCACGAGGCGTTCG | | |
| | | | GCCTCGGCGGCGACGACGAAGCCGTGGTGTACGCGGGA | | |
| | | | ACGGGGCGCTGA | | |
| lysC | Amycolatopsis | AF134837 | GTGGCCCTCGTGGTCCAGAAGTACGGCGGATCGTCGCT | 31 | |
| | mediterranei | | GGAAAGTGCCGACCGGATCAAGCGCGTGGCGGAGCGGA | | |
| | | | TCGTCGCGACGAAGAAGGCGGGCAACGACGTCGTCGTC | | |
| | | | GTCTGCTCGGCGATGGGTGACACCACCGACGAGCTGCT | | |
| | | | CGACCTGGCGCAGCAGGTCAACCCGGCGCCGCCGGAGC | | |
| | | | GGGAGATGGACATGCTGCTCACCGCCGGTGAGCGCATC | | |
| | | | TCGAACTCGCTGGTCGCGATGGCGATCGCGGCCCAGGG | | |
| | | | CGCCGAGGCGTGGTCGTTCACCGGTTCGCAGGCCGGCG | | |
| | | | TCGTCACGACGTCGGTGCACGGCAACGCGCGCATCATC | | |
| | | | GACGTCACGCCGAGCCGGGTCACCGAGGCGCTCGACCA | | |
| | | | GGGGTACATCGCGCTGGTGGCGGGCTTCCAGGGCGTCG | | |
| | | | CGCAGGACACCAAGGACATCACCACGCTGGGCCGCGGC | | |
| | | | GGCTCGGACACCACCGCCGTCGCGCTGGCCGCCGCGCT | | |
| | | | GAACGCCGACGTCTGCGAGATCTACTCCGATGTGGACG | | |
| | | | GTGTGTACACGGCGGACCCGCGGGTGGTGCCGGACGCG | | |
| | | | AAGAAGCTCGACACCGTCACGTACGAAGAGATGCTCGA | | |
| | | | GCTCGCCGCGAGCGGGTCGAAGATCCTGCACCTGCGTT | | |
| | | | CGGTCGAGTACGCGCGCCGCTACGGCGTCCCGATCCGA | | |
| | | | GTCCGTTCTTCCTACAGCGACAAGCCGGGCACGACGGT | | |
| | | | GACCGGTTCTATCGAGGAGATCCCCGTGGAACAAGCCC | | |
| | | | TGATCACCGGTGTGGCGCACGACCGCTCCGAAGCCAAG | | |
| | | | ATCACGGTCACCGGGGTGCCGGACCACACCGGCGCCGC | | |
| | | | GGCCCGGATCTTCCGCGTGATCGCCGACGCCGAGATCG | | |
| | | | ACATCGACATGGTGCTGCAGAACGTGTCCAGCACCGTC | | |
| | | | TCCGGCCGCACGGACATCACGTTCACGCTGTCGAAGGC | | |
| | | | CAACGGCGCCAAGGCCGTCAAGGAACTGGAGAAGGTCC | | |
| | | | AGGCGGAGATCGGCTTCGAGTCGGTCCTCTACGACGAC | | |
| | | | CACGTCGGCAAGGTGTCGGTGGTCGGCGCCGGGATGCG | | |
| | | | CTCGCACCCGGGTGTCACGGCGACGTTCTGCGAAGCGC | | |
| | | | TGGCCGAGGCCGGCGTCAACATCGAAATCATCAACACC | | |
| | | | TCGGAGATCCGCATTTCGGTGCTGATCCGCGACGCGCA | | |
| | | | GCTCGACGACGCCGTGCGCGCGATCCACGAGGCATTCG | | |
| | | | AACTCGGCGGCGACGAAGAAGCCGTCGTCTACGCGGGG | | |
| | | | AGTGGTCGCTGA | | |
| lysC | Streptomyces | AL939117.1 | GTGGGCCTTGTCGTGCAGAAGTACGGAGGCTCCTCCGT | 32 | |
| | coelicolor | | AGCCGATGCCGAGGGCATCAAGCGCGTCGCCAAGCGGA | | |
| | | | TCGTGGAAGCGAAGAAGAACGGCAACCAGGTGGTCGCC | | |
| | | | GTCGTTTCCGCGATGGGCGACACGACGGACGAGCTGAT | | |
| | | | CGATCTCGCCGAGCAGGTTTCCCCGATCCCTGCCGGGC | | |
| | | | GTGAACTCGACATGCTGCTGACCGCCGGGGAGCGTATC | | |
| | | | TCCATGGCGCTGCTGGCCATGGCGATCAAAAACCTGGG | | |
| | | | CCACGAGGCCCAGTCGTTCACCGGCAGCCAGGCCGGAG | | |
| | | | TCATCACCGACTCGGTCCACAACAAGGCCCGGATCATC | | |
| | | | GACGTCACACCGGGTCGCATCCGCACCTCGGTCGACGA | | |
| | | | GGGCAACGTGGCCATCGTGGCCGGCTTCCAGGGCGTCA | | |
| | | | GCCAGGACAGCAAGGACATCACCACGCTGGGCCGCGGC | | |
| | | | GGGTCCGACACCACGGCCGTCGCCCTCGCCGCCGCGCT | | |
| | | | CGACGCGGACGTCTGCGAGATCTACACCGACGTCGACG | | |
| | | | GCGTGTTCACCGCCGACCCGCGCGTGGTGCCGAAGGCG | | |
| | | | AAGAAGATCGACTGGATCTCCTTCGAGGACATGCTGGA | | |
| | | | GCTCGCTGCCTCCGGCTCCAAGGTGCTGCTCCACCGTT | | |
| | | | GCGTGGAGTACGCCCGCCGGTACAACATCCCGATTCAC | | |
| | | | GTGCGGTCCAGCTTCAGCGGACTCCAGGGCACGTGGGT | | |
| | | | CAGCAGCGAGCCGATCAAGCAAGGGGAAAAGCACGTGG | | |
| | | | AGCAGGCCCTCATCTCCGGAGTCGCGCACGACACCTCC | | |
| | | | GAGGCCAAGGTCACGGTCGTCGGGGTGCCCGACAAGCC | | |
| | | | GGGCGAGGCGGCCGCGATCTTCCGCGCCATCGCCGACG | | |
| | | | CCCAGGTCAACATCGACATGGTCGTGCAGAACGTGTCC | | |
| | | | GCCGCCTCCACGGGCCTGACGGACATCTCGTTCACGCT | | |
| | | | CCCCAAGAGCGAGGGCCGCAAGGCCATCGACGCGCTGG | | |
| | | | AGAAGAACCGCCCGGGCATCGGCTTCGACTCGCTGCGC | | |
| | | | TACGACGACCAGATCGGCAAGATCTCGCTGGTCGGCGC | | |
| | | | CGGTATGAAGAGCAATCCGGGCGTCACCGCCGACTTCT | | |
| | | | TCACCGCGCTCTCCGACGCCGGGGTGAACATCGAGCTG | | |
| | | | ATCTCGACCTCCGAGATCCGCATCTCGGTCGTCACCCG | | |
| | | | CAAGGACGACGTGAACGAGGCCGTGCGCGCCGTGCACA | | |
| | | | CCGCCTTCGGGCTCGACTCCGACAGTGACGAGGCCGTG | | |
| | | | GTCTACGGGGGCACCGGGCGCTGA | | |
| lysC | Thermobifida | NZ_AAAQ010 | GTGAATCTCCGATCACTAGACTGGCTGGTCGATTACCG | 33 | |
| | fusca | 00023.1 | TGAACCCGATTCCTCAGGAGCGCCGACCGTGGCTTTGA | | |
| | | | TCGTGCAAAAGTACGGCGGGTCGTCCGTCGCTGATGCG | | |
| | | | GATGCCATTAAGCGGGTAGCCGAACGGATCGTCGCTCA | | |
| | | | GAAGAAAGCCGGATACGACGTGGTCGTCGTGGTCTCCG | | |
| | | | CCATGGGCGACACCACTGACGAGCTTCTCGACCTTGCG | | |
| | | | AAGCAGGTGAGTCCGCTCCCGCCGGGCCGGGAGTTGGA | | |
| | | | CATGCTGCTGACTGCCGGGGAGCGGATCTCGATGGCCC | | |
| | | | TGGTTGCGATGGCTATCGGGAACTTGGGCTATGAGGCC | | |
| | | | CGGTCGTTCACCGGTTCGCAGGCCGGGGTGATCACCAC | | |
| | | | GTCGCTGCACGGCAACGCGAAGATCATCGATGTCACCC | | |
| | | | CGGGGCGGATCAGGGATGCGCTCGCCGAAGGGGCGATC | | |
| | | | TGCATCGTCGCTGGCTTCCAAGGGGTGTCGCAGGACAG | | |
| | | | CAAGGACATCACCACGTTGGGCCGCGGTGGTTCGGACA | | |
| | | | CTACGGCTGTGGCGCTTGCTGCGGCGCTCAACGCCGAC | | |
| | | | TTGTGCGAGATCTACACCGACGTCGACGGGGTGTTCAC | | |
| | | | TGCTGATCCGCGTATCGTGCCCTCCGCTCGACGCATCC | | |
| | | | CCCAGATCTCCTACGAGGAGATGCTGGAGATGGCGGCC | | |
| | | | TCCGGCGCCAAGATCCTGCATCTGCGCTGCGTGGAGTA | | |
| | | | TGCGCGGCGGTACAACATTCCGCTGCACGTGCGCTCGT | | |
| | | | CTTTCAGTCAGAAGCCCGGTACCTGGGTCGTCTCGGAA | | |
| | | | GTTGAGGAAACCGAAGGCATGGAACAACCGATCATCTC | | |
| | | | CGGCGTGGCGCATGACCGGAGCGAAGCCAAGATCACGG | | |
| | | | TTGTGGGGGTGCCCGACCGTGTCGGCGAGGCAGCAGCG | | |
| | | | ATCTTCAAGGCGCTGGCCGACGCTGAGATCAACGTGGA | | |
| | | | CATGATCGTGCAGAACGTGTCCGCGGCTTCCACGTCGC | | |
| | | | GTACGGACATTTCTTTCACTCTGCCTGCCGACTCGGGG | | |
| | | | CAGAACGCGCTGGCCGCGTTGAAGAAGATCCAGGACAA | | |
| | | | GGTCGGTTTCGAGTCGCTGCTGTACAACGACCGGATCG | | |
| | | | GCAAGGTGTCGCTGATCGGCGCGGGGATGCGCTCCTAT | | |
| | | | CCGGGGGTGACTGCTCGGTTCTTTGACGCTGTGGCCCG | | |
| | | | CGAGGGCATCAACATCGAGATGATTTCCACTTCCGAGA | | |
| | | | TCCGCATCTCGATCGTGGTGGCGCAGGACGACGTGGAC | | |
| | | | GCCGCAGTGGCCGCCGCGCACCGTGAGTTCCAGTTGGA | | |
| | | | CGCCGACCAGGTCGAGGCCGTTGTGTATGGAGGTACCG | | |
| | | | GCCGATGA | | |
| lysC | Erwinia | | ATGTCTGCTAACACTGATAACTCACTGATTATCGCCAA | 34 | |
| | chrysanthemi | | ATTCGGCGGCACCAGCGTCGCTGATTTCGACGCCATGA | | |
| | | | ACCGCAGCGCCGACATCGTGCTGTCCGACGCGCAGGTA | | |
| | | | CGGGTGGTGGTGCTGTCCGCCTCCGCCGGCGTGACCAA | | |
| | | | CCTGCTGGTGGCGCTGGCGGAAGGTTTACCGCCATCTG | | |
| | | | AACGCACCGCGCAACTGGAAAAACTGCGCCAGATTCAA | | |
| | | | TACGCCATCATCGACCGCCTCAACCAGCCGGCCGTCAT | | |
| | | | CCGTGAAGAAATCGACCGCATGCTGGACAACGTGGCCC | | |
| | | | GCCTGTCGGAAGCGGCGGCGCTGGCGACTTCCAACGCC | | |
| | | | CTGACCGACGAACTGGTCAGCCACGGCGAGCTGATATC | | |
| | | | CACCTTGCTGTTTGTGGAAATTCTGCGCGAGCGCAACG | | |
| | | | TCGCCGCCGAATGGTTCGACGTGCGTAAAATCATGCGT | | |
| | | | ACCAACGACCGCTTCGGCCGCGCCGAGCCGGACTGCGA | | |
| | | | CGCGCTGGGCGAACTGACCCGCAGCCAGCTGACGCCGC | | |
| | | | GTCTGGCGCAGGGGCTGATCATCACCCAGGGCTTCATC | | |
| | | | GGCAGCGAAGCTAAAGGCCGCACCACCACGCTGGGCCG | | |
| | | | CGGCGGCAGCGATTACACCGCCGCTCTGCTGGGCGAAG | | |
| | | | CGCTGCACGCCAGCCGTATCGACATCTGGACCGACGTT | | |
| | | | CCCGGCATCTACACCACCGACCCGCGCGTGGTGCCGTC | | |
| | | | CGCCCACCGCATCGACCAGATTACCTTTGAAGAAGCGG | | |
| | | | CCGAAATGGCCACCTTCGGCGCCAAGGTGCTGCACCCG | | |
| | | | GCCACACTGCTGCCTGCCGTACGCAGCGACATTCCGGT | | |
| | | | ATTCGTCGGCTCCAGCAAAGACCCGGCGGCCGGCGGCA | | |
| | | | CGCTGGTGTGCAACAACACCGAAAACCCGCCGCTGTTC | | |
| | | | CGCGCGCTGGCGCTGCGCCGCAAGCAGACGCTGCTGAC | | |
| | | | CCTGCATAGCCTTAACATGCTGCACGCGCGCGGCTTTC | | |
| | | | TGGCGGAAGTGTTCAGTATTCTGGCTCGCCACAACATC | | |
| | | | TCGGTGGATTTGATCACTACCTCCGAGGTGAACGTCGC | | |
| | | | GCTGACGCTGGACACCACCGGCTCGACCTCGACCGGCG | | |
| | | | ATAGCCTGCTGTCCAGCGCGCTGCTGACTGAACTGTCC | | |
| | | | TCGCTGTGTCGGGTGGAAGTGGAAGAGAACATGTCGCT | | |
| | | | GGTGGCGCTGATCGGCAACCAGCTGTCGCAGGCCTGCG | | |
| | | | GCGTCGGCAAAGAGGTGTTCGGGGTGCTGGAGCCATTT | | |
| | | | AATATCCGCCTCATCTGCTACGGCGCCAGCAGCCACAA | | |
| | | | CCTGTGCTTCCTGGTGCCGTCCAGCGATGCCGAGCAGG | | |
| | | | TGGTGCAGACGCTGCATCACAATCTGTTTGAATAA | | |
| lysC | Shewanella | AE015779.1 | GTGCTCGAAAAACGAAAGCTTAGTGGTAGCAAGCTTTT | 35 | |
| | oneidensis | | TGTGAAGAAGTTTGGTGGCACTTCGGTGGGTTCAATTG | | |
| | | | AACGTATCGAAGTGGTTGCCGAACAGATTGCAAAGTCC | | |
| | | | GCTCACAGTGGTGAGCAGCAAGTATTAGTTCTTTCTGC | | |
| | | | TATGGCAGGGGAGACAAATAGGCTATTTGCGCTAGCAG | | |
| | | | CGCAAATCGATCCCCGCGCGAGTGCTCGGGAACTCGAT | | |
| | | | ATGTTGGTCTCAACGGGTGAGCAAATTAGTATTGCGTT | | |
| | | | GATGGCGATGGCGTTGCAGCGTCGCGGTATCAAGGCAA | | |
| | | | GATCGCTCACTGGCGATCAAGTGCAAATCCATACAAAT | | |
| | | | AGTCAGTTTGGTCGTGCCAGTATTGAGAGCGTCGATAC | | |
| | | | GGCGTACTTAACGTCCTTGCTCGAACAAGGCATTGTGC | | |
| | | | CGATTGTGGCAGGGTTTCAAGGGATCGATCCTAATGGC | | |
| | | | GATGTCACAACCTTAGGTCGTGGTGGTTCCGATACGAC | | |
| | | | GGCTGTAGCGCTCGCCGCAGCGTTAAGAGCCGATGAAT | | |
| | | | GCCAGATATTTACCGATGTTTCAGGGGTGTTTACTACA | | |
| | | | GACCCAAATATCGATAGTAGCGCAAGGCGTCTGGATGT | | |
| | | | GATTGGCTTTGACGTCATGCTTGAAATGGCAAAGTTAG | | |
| | | | GCGCTAAAGTACTTCATCCTGATTCTGTTGAATATGCA | | |
| | | | CAGCGTTTTAAAGTACCGCTTCGGGTGTTGTCGAGTTT | | |
| | | | CGAAGCTGGGCAAGGTACATTAATTCAATTTGGTGATG | | |
| | | | AATCTGAGCTTGCGATGGCCGCATCTGTACAAGGTATT | | |
| | | | GCGATCAACAAAGCCTTAGCAACGTTGACCATCGAAGG | | |
| | | | TTTGTTCACCAGCAGTGAGCGTTACCAAGCACTATTGG | | |
| | | | CTTGTTTGGCCCGACTGGAGGTAGATGTTGAATTTATC | | |
| | | | ACTCCTTTGAAATTGAATGAAATTTCTCCTGTTGAGTC | | |
| | | | AGTCAGTTTCATGTTAGCCGAAGCTAAAGTGGATATTT | | |
| | | | TATTGCACGAGCTTGAGGTTTTAAGCGAAAGTCTTGAT | | |
| | | | CTAGGGCAATTGATTGTTGAGCGCCAACGTGCAAAAGT | | |
| | | | GTCTTTAGTTGGCAAAGGTTTACAGGCAAAAGTTGGAT | | |
| | | | TATTGACTAAGATGTTAGATGTATTGGGTAACGAAACA | | |
| | | | ATTCATGCTAAGTTACTTTCGACATCGGAGAGTAAATT | | |
| | | | GTCAACTGTGATCGATGAAAGGGACTTGCACAAGGCGG | | |
| | | | TTCGGGCGTTGCATCATGCTTTCGAGCTAAATAAGGTG | | |
| lysC | Coryne- | AX720328 | GTGGCCCTGGTCGTACAGAAATATGGCGGTTCCTCGCT | 238 | |
| | bacterium | | TGAGAGTGCGGAACGCATTAGAAACGTCGCTGAACGGA | | |
| | glutamicum | | TCGTTGCCACCAAGAAGGCTGGAAATGATGTCGTGGTT | | |
| | | | GTCTGCTCCGCAATGGGAGACACCACGGATGAACTTCT | | |
| | | | AGAACTTGCAGCGGCAGTGAATCCCGTTCCGCCAGCTC | | |
| | | | GTGAAATGGATATGCTCCTGACTGCTGGTGAGCGTATT | | |
| | | | TCTAACGCTCTCGTCGCCATGGCTATTGAGTCCCTTGG | | |
| | | | CGCAGAAGCCCAATCTTTCACGGGCTCTCAGGCTGGTG | | |
| | | | TGCTCACCACCGAGCGCCACGGAAACGCACGCATTGTT | | |
| | | | GATGTCACTCCAGGTCGTGTGCGTGAAGCACTCGATGA | | |
| | | | GGGCAAGATCTGCATTGTTGCTGGTTTCCAGGGTGTTA | | |
| | | | ATAAAGAAACCCGCGATGTCACCACGTTGGGTCGTGGT | | |
| | | | GGTTCTGACACCACTGCAGTTGCGTTGGCAGCTGCTTT | | |
| | | | GAACGCTGATGTGTGTGAGATTTACTCGGACGTTGACG | | |
| | | | GTGTGTATACCGCTGACCCGCGCATCGTTCCTAATGCA | | |
| | | | CAGAAGCTGGAAAAGCTCAGCTTCGAAGAAATGCTGGA | | |
| | | | ACTTGCTGCTGTTGGCTCCAAGATTTTGGTGCTGCGCA | | |
| | | | GTGTTGAATACGCTCGTGCATTCAATGTGCCACTTCGC | | |
| | | | GTACGCTCGTCTTATAGTAATGATCCCGGCACTTTGAT | | |
| | | | TGCCGGCTCTATGGAGGATATTCCTGTGGAAGAAGCAG | | |
| | | | TCCTTACCGGTGTCGCAACCGACAAGTCCGAAGCCAAA | | |
| | | | GTAACCGTTCTGGGTATTTCCGATAAGCCAGGCGAGGC | | |
| | | | TGCGAAGGTTTTCCGTGCGTTGGCTGATGCAGAAATCA | | |
| | | | ACATTGACATGGTTCTGCAGAACGTCTCTTCTGTAGAA | | |
| | | | GACGGCACCACCGACATCACCTTCACCTGCCCTCGTTC | | |
| | | | CGACGGCCGCCGCGCGATGGAGATCTTGAAGAAGCTTC | | |
| | | | AGGTTCAGGGCAACTGGACCAATGTGCTTTACGACGAC | | |
| | | | CAGGTCGGCAAAGTCTCCCTCGTGGGTGCTGGCATGAA | | |
| | | | GTCTCACCCAGGTGTTACCGCAGAGTTCATGGAAGCTC | | |
| | | | TGCGCGATGTCAACGTGAACATCGAATTGATTTCCACC | | |
| | | | TCTGAGATTCGTATTTCCGTGCTGATCCGTGAAGATGA | | |
| | | | TCTGGATGCTGCTGCACGTGCATTGCATGAGCAGTTCC | | |
| | | | AGCTGGGCGGCGAAGACGAAGCCGTCGTTTATGCAGGC | | |
| | | | ACCGGACGC | | |
| aspartokinase | Escherichia | M11812 | ATGTCTGAAATTGTTGTCTCCAAATTTGGCGGTACCAG | 239 | |
| III | coli | | CGTAGCCGATTTTGACGCCATGAACCGCAGCGCTGATA | | |
| | | | TTGTGCTTTCTGATGCCAACGTGCGTTTAGTTGTCCTC | | |
| | | | TCGGCTTCTGCTGGTATCACTAATCTGCTGGTCGCTTT | | |
| | | | AGCTGAAGGACTGGAACCTTGCGAGCGATTCGAAAAAC | | |
| | | | TCGACGCTATCCGCAACATCCAGTTTGCCATTCTGGAA | | |
| | | | CGTCTGCGTTACCCGAACGTTATCCGTGAAGAGATTGA | | |
| | | | ACGTCTGCTGGAGAACATTACTGTTCTGGCAGAAGCGG | | |
| | | | CGGCGCTGGCAACGTCTCCGGCGCTGACAGATGAGCTG | | |
| | | | GTCAGCCACGGCGAGCTGATGTCGACCCTGCTGTTTGT | | |
| | | | TGAGATCCTGCGCGAACGCGATGTTCAGGCACAGTGGT | | |
| | | | TTGATGTGCGTAAAGTGATGCGTACCAACGACCGATTT | | |
| | | | GGTCGTGCAGAGCCAGATATAGCCGCGCTGGCGGAACT | | |
| | | | GGCCGCGCTGCAGCTGCTCCCACGTCTCAATGAAGGCT | | |
| | | | TAGTGATCACCCAGGGATTTATCGGTAGCGAAAATAAA | | |
| | | | GGTCGTACAACGACGCTTGGCCGTGGAGGCAGCGATTA | | |
| | | | TACGGCAGCCTTGCTGGCGGAGGCTTTACACGCATCTC | | |
| | | | GTGTTGATATCTGGACCGACGTCCCGGGCATCTACACC | | |
| | | | ACCGATCCACGCGTAGTTTCCGCAGCAAAACGCATTGA | | |
| | | | TGAAATCGCGTTTGCCGAAGCGGCAGAGATGGCAACTT | | |
| | | | TTGGTGCAAAAGTACTGCATCCGGCAACGTTGCTACCC | | |
| | | | GCAGTACGCAGCGATATCCCGGTCTTTGTCGGCTCCAG | | |
| | | | CAAAGACCCACGCGCAGGTGGTACGCTGGTGTGCAATA | | |
| | | | AAACTGAAAATCCGCCGCTGTTCCGCGCTCTGGCGCTT | | |
| | | | CGTCGCAATCAGACTCTGCTCACTTTGCACAGCCTGAA | | |
| | | | TATGCTGCATTCTCGCGGTTTCCTCGCGGAAGTTTTCG | | |
| | | | GCATCCTCGCGCGGCATAATATTTCGGTAGACTTAATC | | |
| | | | ACCACGTCAGAAGTGAGCGTGGCATTAACCCTTGATAC | | |
| | | | CACCGGTTCAACCTCCACTGGCGATACGTTGCTGACAC | | |
| | | | AATCTCTGCTGATGGAGCTTTCCGCACTGTGTCGGGTG | | |
| | | | GAGGTGGAAGAAGGTCTGGCGCTGGTCGCGTTGATTGG | | |
| | | | CAATGACCTGTCAAAAGCGTGCGCCGTTGGCAAAGAGG | | |
| | | | TATTCGGCGTACTGGAACCGTTCAACATTCGCATGATT | | |
| | | | TGTTATGGCGCATCCAGCCATAACCTGTGCTTCCTGGT | | |
| | | | GCCCGGCGAAGATGCCGAGCAGGTGGTGCAAAAACTGC | | |
| | | | ATAGTAATTTGTTTGAGTAA | | |
| asd | Coryne- | X57226 | ATGACCACCATCGCAGTTGTTGGTGCAACCGGCCAGGT | 240 | |
| | bacterium | | CGGCCAGGTTATGCGCACCCTTTTGGAAGAGCGCAATT | | |
| | glutamicum | | TCCCAGCTGACACTGTTCGTTTCTTTGCTTCCCCACGT | | |
| | | | TCCGCAGGCCGTAAGATTGAATTCCGTGGCACGGAAAT | | |
| | | | CGAGGTAGAAGACATTACTCAGGCAACCGAGGAGTCCC | | |
| | | | TCAAGGACATCGACGTTGCGTTGTTCTCCGCTGGAGGC | | |
| | | | ACCGCTTCCAAGCAGTACGCTCCACTGTTCGCTGCTGC | | |
| | | | AGGCGCGACTGTTGTGGATAACTCTTCTGCTTGGCGCA | | |
| | | | AGGACGACGAGGTTCCACTAATCGTCTCTGAGGTGAAC | | |
| | | | CCTTCCGACAAGGATTCCCTGGTCAAGGGCATTATTGC | | |
| | | | GAACCCTAACTGCACCACCATGGCTGCGATGCCAGTGC | | |
| | | | TGAAGCCACTTCACGATGCCGCTGGTCTTGTAAAGCTT | | |
| | | | CACGTTTCCTCTTACCAGGCTGTTTCCGGTTCTGGTCT | | |
| | | | TGCAGGTGTGGAAACCTTGGCAAAGCAGGTTGCTGCAG | | |
| | | | TTGGAGACCACAACGTTGAGTTCGTCCATGATGGACAG | | |
| | | | GCTGCTGACGCAGGCGATGTCGGACCTTATGTTTCACC | | |
| | | | AATCGCTTACAACGTGCTGCCATTCGCCGGAAACCTCG | | |
| | | | TCGATGACGGCACCTTCGAAACCGATGAAGAGCAGAAG | | |
| | | | CTGCGCAACGAATCCCGCAAGATTCTCGGTCTCCCAGA | | |
| | | | CCTCAAGGTCTCAGGCACCTGCGTTCGCGTGCCGGTTT | | |
| | | | TCACCGGCCACACGCTGACCATTCACGCCGAATTCGAC | | |
| | | | AAGGCAATCACCGTGGACCAGGCGCAGGAGATCTTGGG | | |
| | | | TGCCGCTTCAGGCGTCAAGCTTGTCGACGTCCCAACCC | | |
| | | | CACTTGCAGCTGCCGGCATTGACGAATCCCTCGTTGGA | | |
| | | | CGCATCCGTCAGGACTCCACTGTCGACGATAACCGCGG | | |
| | | | TCTGGTTCTCGTCGTATCTGGCGACAACCTCCGCAAGG | | |
| | | | GTGCTGCGCTAAACACCATCCAGATCGCTGAGCTGCTG | | |
| | | | GTTAAGTAA | | |
| asd | Escherichia | NC_000913 | ATGAAAAATGTTGGTTTTATCGGCTGGCGCGGTATGGT | 241 | |
| | coli | | CGGCTCCGTTCTCATGCAACGCATGGTTGAAGAGCGCG | | |
| | | | ACTTCGACGCCATTCGCCCTGTCTTCTTTTCTACTTCT | | |
| | | | CAGCTTGGCCAGGCTGCGCCGTCTTTTGGCGGAACCAC | | |
| | | | TGGCACACTTCAGGATGCCTTTGATCTGGAGGCGCTAA | | |
| | | | AGGCCCTCGATATCATTGTGACCTGTCAGGGCGGCGAT | | |
| | | | TATACCAACGAAATCTATCCAAAGCTTCGTGAAAGCGG | | |
| | | | ATGGCAAGGTTACTGGATTGACGCAGCATCGTCTCTGC | | |
| | | | GCATGAAAGATGACGCCATCATCATTCTTGACCCCGTC | | |
| | | | AATCAGGACGTCATTACCGACGGATTAAATAATGGCAT | | |
| | | | CAGGACTTTTGTTGGCGGTAACTGTACCGTAAGCCTGA | | |
| | | | TGTTGATGTCGTTGGGTGGTTTATTCGCCAATGATCTT | | |
| | | | GTTGATTGGGTGTCCGTTGCAACCTACCAGGCCGCTTC | | |
| | | | CGGCGGTGGTGCGCGACATATGCGTGAGTTATTAACCC | | |
| | | | AGATGGGCCATCTGTATGGCCATGTGGCAGATGAACTC | | |
| | | | GCGACCCCGTCCTCTGCTATTCTCGATATCGAACGCAA | | |
| | | | AGTCACAACCTTAACCCGTAGCGGTGAGCTGCCGGTGG | | |
| | | | ATAACTTTGGCGTGCCGCTGGCGGGTAGCCTGATTCCG | | |
| | | | TGGATCGACAAACAGCTCGATAACGGTCAGAGCCGCGA | | |
| | | | AGAGTGGAAAGGGCAGGCGGAAACCAACAAGATCCTCA | | |
| | | | ACACATCTTCCGTAATTCCGGTAGATGGTTTATGTGTG | | |
| | | | CGTGTCGGGGCATTGCGCTGCCACAGCCAGGCATTCAC | | |
| | | | TATTAAATTGAAAAAAGATGTGTCTATTCCGACCGTGG | | |
| | | | AAGAACTGCTGGCTGCGCACAATCCGTGGGCGAAAGTC | | |
| | | | GTTCCGAACGATCGGGAAATCACTATGCGTGAGCTAAC | | |
| | | | CCCAGCTGCCGTTACCGGCACGCTGACCACGCCGGTAG | | |
| | | | GCCGCCTGCGTAAGCTGAATATGGGACCAGAGTTCCTG | | |
| | | | TCAGCCTTTACCGTGGGCGACCAGCTGCTGTGGGGGGC | | |
| | | | CGCGGAGCCGCTGCGTCGGATGCTTCGTCAACTGGCG | | |
| ppc | Thermobifida | NZ_AAAQ010 | ATGACACGCGACAGCGCCCGCCAGGAGATGCCCGACCA | 36 | |
| | fusca | 00037.1 | GCTTCGCCGCGACGTCCGGTTGCTCGGCGAAATGCTCG | | |
| | | | GCACCGTACTTGCCGAGAGTGGCGGTCAAGACCTGCTT | | |
| | | | GACGATGTGGAACGACTCCGCCGCGCCGTCATCGGAGC | | |
| | | | TCGCGAGGGGACGGTCGAGGGCAAAGAGATCACCGAGC | | |
| | | | TCGTCGCCTCGTGGCCACTGGAACGCGCCAAGCAGGTG | | |
| | | | GCGCGTGCCTTCACCGTCTACTTCCACCTGGTCAACCT | | |
| | | | GGCTGAAGAGCACCACCGTATGCGCGCCCTGCGGGAAC | | |
| | | | GCGACGACGCGGCCACACCGCAGCGCGAATCGCTGGCT | | |
| | | | GCCGCAGTGCACTCCATCCGCGAAGACGCCGGGCCAGA | | |
| | | | GCGGCTGCGCGAACTCATCGCGGGCATGGAATTCCACC | | |
| | | | CGGTCCTGACCGCGCACCCCACCGAAGCGCGCCGTCGC | | |
| | | | GCCGTCTCCACCGCGATCCAGCGCATCAGTGCCCAACT | | |
| | | | GGAACGCCTGCACGCGGCCCACCCGGGAAGCGGCGCCG | | |
| | | | AAGCCGAGGCGCGTCGCAGACTCCTCGAAGAAATCGAC | | |
| | | | CTGCTGTGGCGAACATCACAGCTCCGCTATACGAAGAT | | |
| | | | GGACCCGCTCGACGAAGTGCGGACCGCCATGGCCGCCT | | |
| | | | TCGACGAGACCATCTTCACCGTCATCCCCGAGGTCTAC | | |
| | | | CGCAGCCTCGACCGGGCGCTCGACCCCGAAGGCTGCGG | | |
| | | | ACGGCGCCCCGCGCTGGCGAAAGCCTTCGTCCGCTACG | | |
| | | | GCAGTTGGATCGGCGGTGACCGCGACGGCAACCCCTTC | | |
| | | | GTCACCCACGAAGTGACGCGGGAAGCCATCACCATCCA | | |
| | | | GTCCGAGCACGTGCTGCGCGCCCTGGAAAACGCCTGCG | | |
| | | | AACGCATCGGCCGCACCCACACCGAGTACACCGGCCTC | | |
| | | | ACCCCGCCCAGCGCGGAACTGCGCGCCGCGCTGAGCAG | | |
| | | | CGCCCGGGCTGCCTACCCGCGCCTGATGCAGGAGATCA | | |
| | | | TCAAGCGCTCGCCCAACGAACCCCACCGCCAGCTCCTG | | |
| | | | CTGCTCGCCGCGGAACGGCTCCGCGCCACCCGGCTGCG | | |
| | | | CAACGCCGACCTCGGCTACCCCAACCCGGAAGCGTTCC | | |
| | | | TCGCCGACCTGCGGACCGTCCAAGAGTCGCTTGCTGCC | | |
| | | | GCGGGCGCTGTGCGCCAAGCCTACGGCGAACTCCAAAA | | |
| | | | CCTCATCTGGCAGGCCGAAACCTTCGGCTTCCACCTCG | | |
| | | | CGGAACTGGAAATCCGCCAGCACAGCGCAGTCCACGCC | | |
| | | | GCCGCACTCAAGGAGATACGCGCTGGCGGGGAACTGTC | | |
| | | | CGAACGTACCGAGGAAGTCCTCGCCACCCTGCGGGTCG | | |
| | | | TCGCCTGGATTCAGGAGCGGTTCGGCGTGGAAGCATGC | | |
| | | | CGCCGCTACATCGTCAGCTTCACCCAGTCCGCTGACGA | | |
| | | | CATCGCCGCCGTCTACGAGCTCGCCGAGCACGCCATGC | | |
| | | | CCCCGGGCAAGGCGCCCATCCTCGACGTCATCCCGCTC | | |
| | | | TTCGAAACCGGTGCCGACCTGGACGCGGCCCCCCAGGT | | |
| | | | CCTCGACGGCATGCTCCGCCTGCCCGCCGTCCAGCGCC | | |
| | | | GCCTCGAGCAGACCGGCCGCCGCATGGAAGTCATGCTC | | |
| | | | GGCTACAGCGACTCCGCCAAGGACGTCGGCCCGGTCAG | | |
| | | | CGCCACCCTGCGGCTCTACGACGCCCAGGCGCGGCTGG | | |
| | | | CCGAATGGGCGCGCGAGCACGACATCAAACTCACCCTG | | |
| | | | TTCCACGGCCGCGGCGGTGCCCTGGGCCGCGGCGGCGG | | |
| | | | GCCCGCCAACCGGGCCGTCCTCGCCCAGGCCCCCGGAT | | |
| | | | CGGTGGACGGCCGCTTCAAGGTCACCGAGCAGGGCGPA | | |
| | | | GTCATCTTCGCCCGCTACGGTCAGCGGGCGATCGCCCA | | |
| | | | CCGCCACATCGAACAGGTGGGCCACGCCGTGCTCATGG | | |
| | | | CCTCCACCGAAAGCGTGCAGCGGAGAGCCGCCGAGGCA | | |
| | | | GCCGCCCGGTTCCGCGGTATGGCTGACCGCATCGCCGA | | |
| | | | AGCCGCCCACGCCGCCTACCGCGCCCTCGTCGACACTG | | |
| | | | AAGGGTTCGCGGAGTGGTTCTCCCGGGTCAGCCCGTTG | | |
| | | | GAGGAGCTGAGTGAGCTGCGGCTGGGGTCGCGTCCGGC | | |
| | | | GCGCCGCTCGGCTGCCCGCGGCCTCGACGACCTCCGCG | | |
| | | | CTATCCCGTGGGTGTTCGCCTGGACCCAGACCCGGGTC | | |
| | | | AATCTGCCTGGCTGGTACGGGCTCGGCAGCGGCCTGGC | | |
| | | | CGCGGTCGACGACCTGGAAGCGCTGCACACCGCCTACA | | |
| | | | AGGAGTGGCCGCTGTTCGCCTCGCTGCTGGACAACGCC | | |
| | | | GAGATGAGCCTGGCCAAGACCGACCGGGTGATCGCCGA | | |
| | | | GCGCTACCTCGCGCTGGGCGGGCGTCCAGAGCTCACCG | | |
| | | | AACAGGTCCTCGCCGAATACGACCGCACCCGGGAACTG | | |
| | | | GTCCTCAAAGTCACGCGGCACACCCGCCTCCTCGAGAA | | |
| | | | CCGCCGGGTGCTGTCCCGCGCGGTCGACCTGCGCAACC | | |
| | | | CCTACGTGGACGCCCTTTCGCACCTGCAGCTGCGTGCT | | |
| | | | CTGGAAGCCCTGCGCACCGGGGAAGCCGACCGGCTGTC | | |
| | | | CGAGGAGGACCGCAACCACCTGGAACGGCTCCTGCTGC | | |
| | | | TCTCGGTCAACGGTGTGGCCGCAGGGCTCCAGAACACT | | |
| | | | GGG | | |
| ppc | Mycobacterium | AL583919.1 | ATGGTTGAGTTTTCCGATGCTATACTGGAACCGATCGG | 37 | |
| | leprae (can be | | TGCTGTCCAGCGGACTCGAGTCGGTCGCGAGGCGACTG | | |
| | used to clone | | AACCTATGCGGGCCGACATCAGGCTATTGGGTACCATT | | |
| | M. smegmatis | | CTTGGTGATACTCTGCGTGAGCAGAACGGTGATGAGGT | | |
| | gene) | | ATTCGATCTCGTCGAACGAGTCCGGGTCGAGTCGTTCC | | |
| | | | GGGTGCGGCGTTCTGAGATTGATCGGGCCGATATGGCG | | |
| | | | CGTATGTTCTCTGGTCTCGACATTCACCTGGCCATCCC | | |
| | | | GATCATCCGGGCGTTTAGCCATTTCGCATTGTTGGCCA | | |
| | | | ACGTTGCCGAGGACATCCACCGGGAGCGTCGGCGCCAT | | |
| | | | ATTCACCTCGACGCCGGCGAGCCACTGCGGGATAGCAG | | |
| | | | TTTAGCGGCCACTTACGCGAAACTTGATCTGGCAAAAC | | |
| | | | TAGATTCGGCCACCGTGGCAGATGCCCTTACTGGTGCA | | |
| | | | GTGGTCTCGCCGGTGATTACTGCGCATCCCACCGAGAC | | |
| | | | CCGTCGGCGTACCGTATTTGTTACCCAACGCCGGATTA | | |
| | | | CCGAGTTGATGCGGCTGCACGCGGAGGGACACACCGAA | | |
| | | | ACCGCCGATGGCCGCAGCATTGAGCGTGAATTGCGCCG | | |
| | | | TCAAATTCTCACGCTGTGGCAGACGGCATTGATTCGGT | | |
| | | | TGGCGCGATTGCAGATCTCCGACGAGATCGACGTAGGG | | |
| | | | CTGCGATATTACTCTGCCGCGCTTTTCCATGTGATTCC | | |
| | | | GCAGGTGAATTCCGAGGTGCGCAACGCGTTGCGTGCCC | | |
| | | | GGTGGCCCGACGCCGAGCTGCTGTCCGGCCCTATACTG | | |
| | | | CAACCCGGATCGTGGATCGGTGGTGACCGGGACGGAAA | | |
| | | | CCCGAACGTGACTGCCGACGTGGTGCGGCGAGCGACCG | | |
| | | | GCAGCGCTGCCTACACCGTGGTGGCGCACTATTTGGCT | | |
| | | | GAACTCACCCACCTCGAGCAGGAGCTGTCGATGTCGGC | | |
| | | | GCGACTGATAACCGTCACCCCTGAGCTGGCCACGCTGG | | |
| | | | CCGCTAGCTGTCAGGACGCGGCCTGTGCCGACGAGCCG | | |
| | | | TACCGGCGGGCATTGCGGGTGATCCGCGGTCGATTGTC | | |
| | | | CTCGACTGCCGCCCACATCCTGGATCAGCAGCCACCCA | | |
| | | | ACCAGCTTGGTCTGGGTTTGCCACCGTATTCGACGCCA | | |
| | | | GCCGAACTATGTGCCGATCTGGACACCATCGAAGCCTC | | |
| | | | CCTGTGCACGCACGGCGCCGCGTTGTTAGCCGACGATC | | |
| | | | GGTTGGCGCTGTTGCGAGAAGGTGTTGGAGTCTTTGGG | | |
| | | | TTTCACTTGTGCGGTCTGGATATGCGGCAAAATTCCGA | | |
| | | | CGTGCACGAAGAGGTGGTCGCTGAGCTGTTGGCGTGGG | | |
| | | | CCGGGATGCACCAGGACTACAGTTCGTTGCCCGAAGAT | | |
| | | | CAAAGAGTCAAGCTGCTGGTGGCCGAACTCGGTAACCG | | |
| | | | CCGCCCGTTGGTCGGGGATCGTGCGCAATTATCCGATT | | |
| | | | TGGCGCGCGGCGAGCTGGCCGTTCTTGCGGCCGCTGCC | | |
| | | | CACGCCGTTGAGCTCTACGGATCGGCCGCGGTGCCCAA | | |
| | | | CTACATCATCTCGATGTGTCAGTCTGTGTCGGATGTCC | | |
| | | | TGGAGGTCGCGATCCTCTTGAAGGAGACTGGCCTGTTA | | |
| | | | GACGCCTCCGGGTCGCAGCCGTACTGTCCGGTGGGCAT | | |
| | | | CTCGCCGCTGTTCGAGACGATCGACGATCTGCACAACG | | |
| | | | GGGCGGCCATTCTGCACGCGATGCTGGAACTTCCGCTA | | |
| | | | TATCGAACGCTGGTGGCTGCTCGCGGTAACTGGCAGGA | | |
| | | | AGTGATGCTCGGCTACTCCGATTCCAACAAAGATGGCG | | |
| | | | GCTATCTGGCCGCCAACTGGGCGGTTTACCGCGCCGAG | | |
| | | | CTCGCTCTGGTAGACGTGGCCCGCAAAACCGGAATCCG | | |
| | | | TTTGCGACTTTTCCATGGTCGTGGCGGCACTGTCGGAC | | |
| | | | GTGGCGGCGGTCCTAGCTATCAAGCTATTCTGGCGCAA | | |
| | | | CCCCCGGGGGCGGTAAACGGCTCGTTGCGTCTCACCGA | | |
| | | | GCAAGGCGAGGTCATAGCCGCCAAATACGCCGAACCGC | | |
| | | | AAATAGCACGACGAAACCTAGAGAGTTTGGTGGCCGCG | | |
| | | | ACCCTAGAATCAACTCTCTTGGATGTTGAAGGCTTAGG | | |
| | | | CGATGCGGCTGAATCTGCTTACGCCATACTCGATGAAG | | |
| | | | TAGCCGGCCTCGCGCGGCGATCCTACGCTGAATTAGTC | | |
| | | | AACACACCGGGTTTCGTTGACTATTTCCAAGCTTCCAC | | |
| | | | GCCGGTCAGCGAGATCGGATCGTTGAACATTGGCAACC | | |
| | | | GACCGACATCACGTAAGCCTACCACGTCGATCGCGGAT | | |
| | | | CTTCGTGCTATTCCGTGGGTACTGGCATGGAGCCAATC | | |
| | | | GCGAGTCATGCTCCCAGGTTGGTATGGCACCGGATCGG | | |
| | | | CGTTTCAGCAGTGGGTTGCGGCTGGACCCGAAAGTGAA | | |
| | | | TCACAGCGGGTAGAAATGCTGCATGACCTCTATCAGCG | | |
| | | | TTGGCCGTTCTTTCGAAGTGTGCTGTCGAACATGGCGC | | |
| | | | AGGTACTGGCCAAAAGTGATCTGGGCCTGGCGGCCCGC | | |
| | | | TATGCTGAGCTGGTGGTCGACGAAGCCTTGCGGCGCAG | | |
| | | | AGTGTTTGACAAGATCGCCGACGAGCATCGGCGAACCA | | |
| | | | TTGCCATCCACAAGCTCATTACGGGTCATGACGATCTG | | |
| | | | CTTGCTGACAACCCGGCTCTGGCGCGTTCGGTGTTCAA | | |
| | | | CCGCTTCCCGTATCTGGAGCCGTTAAACCACCTTCAGG | | |
| | | | TGGAGCTATTGCGCCGCTACCGCTCGGGTCACGACGAC | | |
| | | | GAAATGGTGCAACGCGGCATCCTTTTGACAATGAACGG | | |
| | | | ATTGGCCAGCGCGCTACGTAACAGCGGC | | |
| ppc | Streptomyces | AF177946.1 | GTGAGCAGTGCCGACGACCAGACCACCACGACGACCAG | 38 | |
| | coelicolor | | CAGTGAACTGCGCGCCGACATCCGCCGGCTGGGTGATC | | |
| | | | TCCTCGGGGAGACCCTGGTCCGGCAGGAGGGCCCCGAA | | |
| | | | CTGCTGGAACTCGTCGAGAAGGTACGCCGACTCACCCG | | |
| | | | AGAGGACGGCGAGGCCGCCGCCGAACTGCTGCGCGGCA | | |
| | | | CCGAACTGGAGACCGCCGCCAAGCTCGTCCGCGCCTTC | | |
| | | | TCCACCTACTTCCACCTGGCCAACGTCACCGAGCAGGT | | |
| | | | CCACCGCGGCCGCGAGCTGGGCGCCAAGCGCGCCGCCG | | |
| | | | AGGGCGGACTGCTCGCCCGTACGGCCGACCGGCTGAAG | | |
| | | | GACGCCGACCCCGAGCACCTGCGCGAGACGGTCCGCAA | | |
| | | | CCTCAACGTGCGCCCCGTGTTCACCGCGCACCCCACCG | | |
| | | | AGGCCGCCCGCCGCTCCGTCCTCAACAAGCTGCGCCGC | | |
| | | | ATCGCCGCCCTCCTGGACACCCCGGTCAACGAGTCGGA | | |
| | | | CCGGCGCCGCCTGGACACCCGCCTCGCCGAGAACATCG | | |
| | | | ACCTCGTCTGGCAGACCGACGAGCTGCGCGTCGTGCGC | | |
| | | | CCCGAGCCCGCCGACGAGGCCCGCAACGCCATCTACTA | | |
| | | | CCTCGACGAGCTGCACCTGGGCGCCGTCGGCGACGTCC | | |
| | | | TCGAAGACCTCACCGCCGAGCTGGAGCGGGCCGGCGTC | | |
| | | | AAGCTCCCCGACGACACCCGCCCCCTCACCTTCGGCAC | | |
| | | | CTGGATCGGCGGCGACCGCGACGGCAACCCCAACGTCA | | |
| | | | CCCCCCAGGTGACCTGGGACGTCCTCATCCTCCAGCAC | | |
| | | | GAGCACGGCATCAACGACGCCCTGGAGATGATCGACGA | | |
| | | | GCTGCGCGGCTTCCTCTCCAACTCCATCCGGTACGCCG | | |
| | | | GTGCGACCGAGGAACTGCTCGCCTCGCTCCAGGCCGAC | | |
| | | | CTGGAACGCCTCCCCGAGATCAGCCCCCGCTACAAGCG | | |
| | | | CCTCAACGCCGAGGAGCCCTACCGGCTCAAGGCCACCT | | |
| | | | GCATCCGCCAGAAGCTGGAGAACACCAAGCAGCGCCTC | | |
| | | | GCCAAGGGCACCCCCCACGAGGACGGCCGCGACTACCT | | |
| | | | CGGCACCGCCCAGCTCATCGACGACCTGCGCATCGTCC | | |
| | | | AGACCTCGCTGCGCGAACACCGCGGCGGCCTGTTCGCC | | |
| | | | GACGGGCGCCTCGCCCGCACCATCCGCACCCTGGCCGC | | |
| | | | CTTCGGCCTCCAGCTCGCCACCATGGACGTCCGCGAGC | | |
| | | | ACGCCGACGCCCACCACCACGCCCTCGGCCAGCTCTTC | | |
| | | | GACCGGCTCGGCGAGGAGTCCTGGCGCTACGCCGACAT | | |
| | | | GCCGCGCGAGTACCGCACCAAGCTCCTCGCCAAGGAAC | | |
| | | | TGCGCTCCCGCAGGCCGCTGGCCCCCAGCCCCGCCCCC | | |
| | | | GTCGACGCGCCCGGCGAGAAGACCCTCGGCGTCTTCCA | | |
| | | | GACCGTCCGCCGCGCCCTGGAGGTCTTCGGCCCCGAGG | | |
| | | | TCATCGAGTCCTACATCATCTCCATGTGCCAGGGCGCC | | |
| | | | GACGACGTCTTCGCCGCGGCGGTACTGGCCCGCGAGGC | | |
| | | | CGGGCTGATCGACCTGCACGCCGGCTGGGCGAAGATCG | | |
| | | | GCATCGTGCCGCTGCTGGAGACCACCGACGAGCTGAAG | | |
| | | | GCCGCCGACACCATCCTGGAGGACCTGCTCGCCGACCC | | |
| | | | CTCCTACCGGCGCCTGGTCGCGCTGCGCGGCGACGTCC | | |
| | | | AGGAGGTCATGCTCGGCTACTCCGACTCCTCCAAGTTC | | |
| | | | GGCGGTATCACCACCAGCCAGTGGGAGATCCACCGCGC | | |
| | | | CCAGCGCCGGCTGCGCGACGTCGCCCACCGCTACGGCG | | |
| | | | TACGGCTGCGCCTCTTCCACGGCCGCGGCGGCACCGTC | | |
| | | | GGCCGCGGCGGCGGCCCCACCCACGACGCCATCCTCGC | | |
| | | | CCAGCCCTGGGGCACCCTGGAGGGCGAGATCAAGGTCA | | |
| | | | CCGAGCAGGGCGAGGTCATCTCCGACAAGTACCTCATC | | |
| | | | CCCGCCCTCGCCCGGGAGAACCTGGAGCTGACCGTCGC | | |
| | | | GGCCACCCTCCAGGCCTCCGCCCTGCACACCGCGCCCC | | |
| | | | GCCAGTCCGACGAGGCCCTGGCCCGCTGGGACGCCGCG | | |
| | | | ATGGACGTCGTCTCCGACGCCGCCCACACCGCCTACCG | | |
| | | | GCACCTGGTCGAGGACCCCGACCTGCCGACCTACTTCC | | |
| | | | TGGCCTCCACCCCGGTCGACCAGCTCGCCGACCTGCAC | | |
| | | | CTGGGCTCGCGGCCCTCCCGCCGCCCCGGCTCGGGCGT | | |
| | | | CTCGCTCGACGGACTGCGCGCCATCCCGTGGGTGTTCG | | |
| | | | GCTGGACCCAGTCCCGGCAGATCGTCCCCGGCTGGTAC | | |
| | | | GGCGTCGGCTCCGGCCTCAAGGCCCTGCGCGAGGCGGG | | |
| | | | CCTGGACACCGTGCTCGACGAGATGCACCAGCAGTGGC | | |
| | | | ACTTCTTCCGCAACTTCATCTCCAACGTCGAGATGACC | | |
| | | | CTCGCCAAGACCGACCTGCGCATCGCCCAGCACTACGT | | |
| | | | CGACACCCTCGTCCCGGACGAGCTCAAGCACGTCTTCG | | |
| | | | ACACCATCAAGGCCGAGCACGAGCTCACCGTCGCCGAG | | |
| | | | GTCCTGCGCGTCACCGGCGAGAGTGAACTGCTGGACGC | | |
| | | | CGACCCGGTCCTCAAGCAGACCTTCACCATCCGCGACG | | |
| | | | CCTACCTCGACCCCATCTCCTACCTCCAGGTCGCCCTC | | |
| | | | CTCGGCCGTCAGCGCGAGGCCGCCGCCGCGAACGAGGA | | |
| | | | CCCGGACCCCCTCCTCGCCCGAGCCCTCCTCCTCACCG | | |
| | | | TCAACGGCGTGGCAGCGGGCCTGCGCAACACCGGCTGA | | |
| ppc | Erwinia | | ATGAATGAACAATATTCCGCCATGCGGAGCAATGTCAG | 39 | |
| | chrysanthemi | | CATGCTGGGTAAACTACTCGGCGACACCATCAAGGATG | | |
| | | | CGCTGGGCGCCAATATCCTTGAGCGTGTTGAAACAATC | | |
| | | | CGCAAGCTGTCCAAAGCCTCGCGGGCCGGCAGCGAAAC | | |
| | | | ACACCGTCAGGAACTGCTGACCACACTGCAGAACCTGT | | |
| | | | CCAACGATGAACTGCTGCCGGTCGCCCGCGCATTCAGC | | |
| | | | CAGTTCCTTAACCTGACCAACACCGCCGAGCAATACCA | | |
| | | | CAGTATCTCTCCGCACGGCGAAGCGGCCAGTAACCCGG | | |
| | | | AAGCGCTGGCGACGGTGTTTCGCAGTCTGAAAAGCCGC | | |
| | | | GACAACCTGAGCGACAAGGATATCCGCGACGCGGTGGA | | |
| | | | GTCGCTCTCCATCGAGCTGGTGTTGACCGCGCACCCGA | | |
| | | | CCGAAATCACCCGCCGTACGCTGATCCACAAACTGGTT | | |
| | | | GAAGTGAATACCTGCCTCAAGCAGCTCGATCACGACGA | | |
| | | | TCTGGCCGATTATGAACGCCACCAGATCATGCGCCGTC | | |
| | | | TGCGCCAGCTGATCGCCCAATACTGGCATACCGATGAA | | |
| | | | ATCCGCAAAATCCGCCCGACGCCGGTGGACGAAGCCAA | | |
| | | | GTGGGGTTTCGCGGTGGTGGAAAATAGCCTGTGGGAAG | | |
| | | | GGGTGCCGGCGTTTCTGCGCGAACTCGACGAGCAGATG | | |
| | | | GGTAAAGAGTTGGGCTACCGTCTGCCGGTGGATTCGGT | | |
| | | | GCCGGTGCGCTTCACCTCCTGGATGGGCGGCGACCGCG | | |
| | | | ACGGCAACCCGAACGTGACCTCTGAAGTCACCCGCCGC | | |
| | | | GTGCTGCTGCTAAGCCGCTGGAAAGCCGCGGACCTGTT | | |
| | | | CCTGCGCGACGTACAGGTGCTGGTTTCCGAACTGTCGA | | |
| | | | TGACCACCTGTACGCCGGAACTGCAACAACTGGCAGGC | | |
| | | | GGCGACGAGGTGCAGGAACCCTACCGCGAACTGATGAA | | |
| | | | AGCGCTGCGCGCACAGTTGACTGCTACCCTGGATTATC | | |
| | | | TGGACGCGCGTCTGAAAGATGAACAACGGATGCCGCCC | | |
| | | | AAAGATCTGCTGGTCACCAACGAGCAGTTATGGGAACC | | |
| | | | GCTGTACGCCTGTTACCAGTCGCTGCATGCCTGCGGCA | | |
| | | | TGGGCATCATCGCCGATGGTCAATTGCTCGATACCCTG | | |
| | | | CGCCGGGTGCGCTGCTTTGGCGTGCCGCTGGTGCGTAT | | |
| | | | CGACGTACGTCAGGAGAGCACCCGTCACACCGACGCGC | | |
| | | | TGGCGGAAATCACCCGCTATCTGGGGCTGGGAGACTAC | | |
| | | | GAAAGCTGGTCGGAATCCGACAAGCAGGCGTTCCTGAT | | |
| | | | CCGCGAACTTAACTCCAAGCGTCCGCTGCTGCCGCGCC | | |
| | | | AGTGGGAACCGAGCGCCGACACCCAGGAAGTGCTGGAA | | |
| | | | ACCTGCCGGGTGATCGCCGAAACCCCGCGCGACTCCAT | | |
| | | | CGCCGCCTATGTAATTTCGATGGCGCGCACCCCGTCCG | | |
| | | | ACGTGCTGGCGGTGCATTTGCTGCTGAAAGAAGCCGGC | | |
| | | | TGTCCGTACGCGCTGCCGGTGGCGCCGCTGTTCGAAAC | | |
| | | | GCTGGACGACCTGAATAACGCCGACAGCGTAATGATCC | | |
| | | | AGTTGCTCAACATCGACTGGTATCGCGGCTTCATTCAG | | |
| | | | GGCAAGCAGATGGTGATGATCGGCTATTCCGACTCCGC | | |
| | | | CAAAGACGCCGGGGTGATGGCGGCCTCCTGGGCGCAGT | | |
| | | | ACCGCGCGCAAGACGCACTGATCAAGACCTGCGAGAAA | | |
| | | | TACGGCATCGCCCTGACGCTGTTTCACGGTCGCGGCGG | | |
| | | | TTCGATTGGCCGCGGCGGCGCGCCGGCTCACGCCGCGC | | |
| | | | TGCTCTCCCAACCGCCGGGCAGCCTGAAAGGCGGCCTG | | |
| | | | CGCGTCACCGAACAGGGCGAGATGATCCGCTTTAAGTT | | |
| | | | CGGCCTGCCGGAAGTCACCATTAGCAGCCTGTCGCTCT | | |
| | | | ACACGTCCGCCATTCTGGAAGCCAACCTGTTGCCGCCG | | |
| | | | CCGGAGCCGAAGCAGGAGTGGCATCACATCATGAACGA | | |
| | | | GCTGTCGCGCATTTCCTGCGACATGTACCGCGGCTACG | | |
| | | | TACGGGAAAACCCGGATTTCGTGCCCTACTTCCGTGCC | | |
| | | | GCCACGCCGGAGCTGGAACTGGGCAAACTGCCGCTGGG | | |
| | | | GTCACGTCCGGCCAAGCGTCGGCCGAACGGCGGCGTGG | | |
| | | | AAAGCCTGCGCGCCATCCCGTGGATTTTCGCCTGGACC | | |
| | | | CAGAACCGCCTGATGCTGCCCGCCTGGTTGGGCGCCGG | | |
| | | | CGCCGCGCTGCAAAAAGTGATCGACGACGGTCACCAGA | | |
| | | | ACCAGCTGGAAGCCATGTGCCGCGACTGGCCGTTCTTC | | |
| | | | TCCACCCGTATCGGTATGCTGGAAATGGTATTCGCCAA | | |
| | | | GGCCGACCTATGGCTGGCGGAATACTACGATCAGCGGC | | |
| | | | TGGTGGACGAGAAACTGTGGTCGCTCGGCAAACAGCTG | | |
| | | | CGCGAACAGCTGGAAAGAGACATCAAAGCGGTGTTGAC | | |
| | | | CATCTCCAACGACGACCATCTGATGGCCGACCTGCCGT | | |
| | | | GGATCGCCGAATCCATCGCGCTACGCAACGTCTACACC | | |
| | | | GACCCGCTCAACGTGCTGCAGGCGGAGCTGCTGCACCG | | |
| | | | TTCACGCCAGCAGGAAACACTGGACCCGCAGGTGGAAC | | |
| | | | AGGCGCTGATGGTCACCATCGCCGGCGTCGCCGCCGGG | | |
| | | | ATGCGCAATACCGGCTAA | | |
| ppc | Coryne- | NC_003450 | ATGACTGATTTTTTACGCGATGACATCAGGTTCCTCGG | 242 | |
| | bacterium | | TCAAATCCTCGGTGAGGTAATTGCGGAACAAGAAGGCC | | |
| | glutamicum | | AGGAGGTTTATGAACTGGTCGAACAAGCGCGCCTGACT | | |
| | | | TCTTTTGATATCGCCAAGGGCAACGCCGAAATGGATAG | | |
| | | | CCTGGTTCAGGTTTTCGACGGCATTACTCCAGCCAAGG | | |
| | | | CAACACCGATTGCTCGCGCATTTTCCCACTTCGCTCTG | | |
| | | | CTGGCTAACCTGGCGGAAGACCTCTACGATGAAGAGCT | | |
| | | | TCGTGAACAGGCTCTCGATGCAGGCGACACCCCTCCGG | | |
| | | | ACAGCACTCTTGATGCCACCTGGCTGAAACTCAATGAG | | |
| | | | GGCAATGTTGGCGCAGAAGCTGTGGCCGATGTGCTGCG | | |
| | | | CAATGCTGAGGTGGCGCCGGTTCTGACTGCGCACCCAA | | |
| | | | CTGAGACTCGCCGCCGCACTGTTTTTGATGCGCAAAAG | | |
| | | | TGGATCACCACCCACATGCGTGAACGCCACGCTTTGCA | | |
| | | | GTCTGCGGAGCCTACCGCTCGTACGCAAAGCAAGTTGG | | |
| | | | ATGAGATCGAGAAGAACATCCGCCGTCGCATCACCATT | | |
| | | | TTGTGGCAGACCGCGTTGATTCGTGTGGCCCGCCCACG | | |
| | | | TATCGAGGACGAGATCGAAGTAGGGCTGCGCTACTACA | | |
| | | | AGCTGAGCCTTTTGGAAGAGATTCCACGTATCAACCGT | | |
| | | | GATGTGGCTGTTGAGCTTCGTGAGCGTTTCGGCGAGGG | | |
| | | | TGTTCCTTTGAAGCCCGTGGTCAAGCCAGGTTCCTGGA | | |
| | | | TTGGTGGAGACCACGACGGTAACCCTTATGTCACCGCG | | |
| | | | GAAACAGTTGAGTATTCCACTCACCGCGCTGCGGAAAC | | |
| | | | CGTGCTCAAGTACTATGCACGCCAGCTGCATTCCCTCG | | |
| | | | AGCATGAGCTCAGCCTGTCGGACCGCATGAATAAGGTC | | |
| | | | ACCCCGCAGCTGCTTGCGCTGGCAGATGCAGGGCACAA | | |
| | | | CGACGTGCCAAGCCGCGTGGATGAGCCTTATCGACGCG | | |
| | | | CCGTCCATGGCGTTCGCGGACGTATCCTCGCGACGACG | | |
| | | | GCCGAGCTGATCGGCGAGGACGCCGTTGAGGGCGTGTG | | |
| | | | GTTCAAGGTCTTTACTCCATACGCATCTCCGGAAGAAT | | |
| | | | TCTTAAACGATGCGTTGACCATTGATCATTCTCTGCGT | | |
| | | | GAATCCAAGGACGTTCTCATTGCCGATGATCGTTTGTC | | |
| | | | TGTGCTGATTTCTGCCATCGAGAGCTTTGGATTCAACC | | |
| | | | TTTACGCACTGGATCTGCGCCAAAACTCCGAAAGCTAC | | |
| | | | GAGGACGTCCTCACCGAGCTTTTCGAACGCGCCCAAGT | | |
| | | | CACCGCAAACTACCGCGAGCTGTCTGAAGCAGAGAAGC | | |
| | | | TTGAGGTGCTGCTGAAGGAACTGCGCAGCCCTCGTCCG | | |
| | | | CTGATCCCGCACGGTTCAGATGAATACAGCGAGGTCAC | | |
| | | | CGACCGCGAGCTCGGCATCTTCCGCACCGCGTCGGAGG | | |
| | | | CTGTTAAGAAATTCGGGCCACGGATGGTGCCTCACTGC | | |
| | | | ATCATCTCCATGGCATCATCGGTCACCGATGTGCTCGA | | |
| | | | GCCGATGGTGTTGCTCAAGGAATTCGGACTCATCGCAG | | |
| | | | CCAACGGCGACAACCCACGCGGCACCGTCGATGTCATC | | |
| | | | CCACTGTTCGAAACCATCGAAGATCTCCAGGCCGGCGC | | |
| | | | CGGAATCCTCGACGAACTGTGGAAAATTGATCTCTACC | | |
| | | | GCAACTACCTCCTGCAGCGCGACAACGTCCAGGAAGTC | | |
| | | | ATGCTCGGTTACTCCGATTCCAACAAGGATGGCGGATA | | |
| | | | TTTCTCCGCAAACTGGGCGCTTTACGACGCGGAACTGC | | |
| | | | AGCTCGTCGAACTATGCCGATCAGCCGGGGTCAAGCTT | | |
| | | | CGCCTGTTCCACGGCCGTGGTGGCACCGTCGGCCGCGG | | |
| | | | TGGCGGACCTTCCTACGACGCGATTCTTGCCCAGCCCA | | |
| | | | GGGGGGCTGTCCAAGGTTCCGTGCGCATCACCGAGCAG | | |
| | | | GGCGAGATCATCTCCGCTAAGTACGGCAACCCCGAAAC | | |
| | | | CGCGCGCCGAAACCTCGAAGCCCTGGTCTCAGCCACGC | | |
| | | | TTGAGGCATCGCTTCTCGACGTCTCCGAACTCACCGAT | | |
| | | | CACCAACGCGCGTACGACATCATGAGTGAGATCTCTGA | | |
| | | | GCTCAGCTTGAAGAAGTACGCCTCCTTGGTGCACGAGG | | |
| | | | ATCAAGGCTTCATCGATTACTTCACCCAGTCCACGCCG | | |
| | | | CTGCAGGAGATTGGATCCCTCAACATCGGATCCAGGCC | | |
| | | | TTCCTCACGCAAGCAGACCTCCTCGGTGGAAGATTTGC | | |
| | | | GAGCCATCCCATGGGTGCTCAGCTGGTCACAGTCTCGT | | |
| | | | GTCATGCTGCCAGGCTGGTTTGGTGTCGGAACCGCATT | | |
| | | | AGAGCAGTGGATTGGCGAAGGGGAGCAGGCCACCCAAC | | |
| | | | GCATTGCCGAGCTGCAAACACTCAATGAGTCCTGGCCA | | |
| | | | TTTTTCACCTCAGTGTTGGATAACATGGCTCAGGTGAT | | |
| | | | GTCCAAGGCAGAGCTGCGTTTGGCAAAGCTCTACGCAG | | |
| | | | ACCTGATCCCAGATACGGAAGTAGCCGAGCGAGTCTAT | | |
| | | | TCCGTCATCCGCGAGGAGTACTTCCTGACCAAGAAGAT | | |
| | | | GTTCTGCGTAATCACCGGCTCTGATGATCTGCTTGATG | | |
| | | | ACAACCCACTTCTCGCACGCTCTGTCCAGCGCCGATAC | | |
| | | | CCCTACCTGCTTCCACTCAACGTGATCCAGGTAGAGAT | | |
| | | | GATGCGACGCTACCGAAAAGGCGACCAAAGCGAGCAAG | | |
| | | | TGTCCCGCAACATTCAGCTGACCATGAACGGTCTTTCC | | |
| | | | ACTGCGCTGCGCAACTCCGGC | | |
| ppc | Escherichia | X05903 | ATGAACGAACAATATTCCGCATTGCGTAGTAATGTCAG | 243 | |
| | coli | | TATGCTCGGCAAAGTGCTGGGAGAAACCATCAAGGATG | | |
| | | | CGTTGGGAGAACACATTCTTGAACGCGTAGAAACTATC | | |
| | | | CGTAAGTTGTCGAAATCTTCACGCGCTGGCAATGATGC | | |
| | | | TAACCGCCAGGAGTTGCTCACCACCTTACAAAATTTGT | | |
| | | | CGAACGACGAGCTGCTGCCCGTTGCGCGTGCGTTTAGT | | |
| | | | CAGTTCCTGAACCTGGCCAACACCGCCGAGCAATACCA | | |
| | | | CAGCATTTCGCCGAAAGGCGAAGCTGCCAGCAACCCGG | | |
| | | | AAGTGATCGCCCGCACCCTGCGTAAACTGAAAAACCAG | | |
| | | | CCGGAACTGAGCGAAGACACCATCAAAAAAGCAGTGGA | | |
| | | | ATCGCTGTCGCTGGAACTGGTCCTCACGGCTCACCCAA | | |
| | | | CCGAAATTACCCGTCGTACACTGATCCACAAAATGGTG | | |
| | | | GAAGTGAACGCCTGTTTAAAACAGCTCGATAACAAAGA | | |
| | | | TATCGCTGACTACGAACACAACCAGCTGATGCGTCGCC | | |
| | | | TGCGCCAGTTGATCGCCCAGTCATGGCATACCGATGAA | | |
| | | | ATCCGTAAGCTGCGTCCAAGCCCGGTAGATGAAGCCAA | | |
| | | | ATGGGGCTTTGCCGTAGTGGAAAACAGCCTGTGGCAAG | | |
| | | | GCGTACCAAATTACCTGCGCGAACTGAACGAACAACTG | | |
| | | | GAAGAGAACCTCGGCTACAAACTGCCCGTCGAATTTGT | | |
| | | | TCCGGTCCGTTTTACTTCGTGGATGGGCGGCGACCGCG | | |
| | | | ACGGCAACCCGAACGTCACTGCCGATATCACCCGCCAC | | |
| | | | GTCCTGCTACTCAGCCGCTGGAAAGCCACCGATTTGTT | | |
| | | | CCTGAAAGATATTCAGGTGCTGGTTTCTGAACTGTCGA | | |
| | | | TGGTTGAAGCGACCCCTGAACTGCTGGCGCTGGTTGGC | | |
| | | | GAAGAAGGTGCCGCAGAACCGTATCGCTATCTGATGAA | | |
| | | | AAACCTGCGTTCTCGCCTGATGGCGACACAGGCATGGC | | |
| | | | TGGAAGCGCGCCTGAAAGGCGAAGAACTGCCAAAACCA | | |
| | | | GAAGGCCTGCTGACACAAAACGAAGAACTGTGGGAACC | | |
| | | | GCTCTACGCTTGCTACCAGTCACTTCAGGCGTGTGGCA | | |
| | | | TGGGTATTATCGCCAACGGCGATCTGCTCGACACCCTG | | |
| | | | CGCCGCGTGAAATGTTTCGGCGTACCGCTGGTCCGTAT | | |
| | | | TGATATCCGTCAGGAGAGCACGCGTCATACCGAAGCGC | | |
| | | | TGGGCGAGCTGACCCGCTACCTCGGTATCGGCGACTAC | | |
| | | | GAAAGCTGGTCAGAGGCCGACAAACAGGCGTTCCTGAT | | |
| | | | CCGCGAACTGAACTCCAAACGTCCGCTTCTGCCGCGCA | | |
| | | | ACTGGCAACCAAGCGCCGAAACGCGCGAAGTGCTCGAT | | |
| | | | ACCTGCCAGGTGATTGCCGAAGCACCGCAAGGCTCCAT | | |
| | | | TGCCGCCTACGTGATCTCGATGGCGAAAACGCCGTCCG | | |
| | | | ACGTACTGGCTGTCCACCTGCTGCTGAAAGAAGCGGGT | | |
| | | | ATCGGGTTTGCGATGCCGGTTGCTCCGCTGTTTGAAAC | | |
| | | | CCTCGATGATCTGAACAACGCCAACGATGTCATGACCC | | |
| | | | AGCTGCTCAATATTGACTGGTATCGTGGCCTGATTCAG | | |
| | | | GGCAAACAGATGGTGATGATTGGCTATTCCGACTCAGC | | |
| | | | AAAAGATGCGGGAGTGATGGCAGCTTCCTGGGCGCAAT | | |
| | | | ATCAGGCACAGGATGCATTAATCAAAACCTGCGAAAAA | | |
| | | | GCGGGTATTGAGCTGACGTTGTTCCACGGTCGCGGCGG | | |
| | | | TTCCATTGGTCGCGGCGGCGCACCTGCTCATGCGGCGC | | |
| | | | TGCTGTCACAACCGCCAGGAAGCCTGAAAGGCGGCCTG | | |
| | | | CGCGTAACCGAACAGGGCGAGATGATCCGCTTTAAATA | | |
| | | | TGGTCTGCCAGAAATCACCGTCAGCAGCCTGTCGCTTT | | |
| | | | ATACCGGGGCGATTCTGGAAGCCAACCTGCTGCCACCG | | |
| | | | CCGGAGCCGAAAGAGAGCTGGCGTCGCATTATGGATGA | | |
| | | | ACTGTCAGTCATCTCCTGCGATGTCTACCGCGGCTACG | | |
| | | | TACGTGAAAACAAAGATTTTGTGCCTTACTTCCGCTCC | | |
| | | | GCTACGCCGGAACAAGAACTGGGCAAACTGCCGTTGGG | | |
| | | | TTCACGTCCGGCGAAACGTCGCCCAACCGGCGGCGTCG | | |
| | | | AGTCACTACGCGCCATTCCGTGGATCTTCGCCTGGACG | | |
| | | | CAAAACCGTCTGATGCTCCCCGCCTGGCTGGGTGCAGG | | |
| | | | TACGGCGCTGCAAAAAGTGGTCGAAGACGGCAAACAGA | | |
| | | | GCGAGCTGGAGGCTATGTGCCGCGATTGGCCATTCTTC | | |
| | | | TCGACGCGTCTCGGCATGCTGGAGATGGTCTTCGCCAA | | |
| | | | AGCAGACCTGTGGCTGGCGGAATACTATGACCAACGCC | | |
| | | | TGGTAGACAAAGCACTGTGGCCGTTAGGTAAAGAGTTA | | |
| | | | CGCAACCTGCAAGAAGAAGACATCAAAGTGGTGCTGGC | | |
| | | | GATTGCCAACGATTCCCATCTGATGGCCGATCTGCCGT | | |
| | | | GGATTGCAGAGTCTATTCAGCTACGGAATATTTACACC | | |
| | | | GACCCGCTGAACGTATTGCAGGCCGAGTTGCTGCACCG | | |
| | | | CTCCCGCCAGGCAGAAAAAGAAGGCCAGGAACCGGATC | | |
| | | | CTCGCGTCGAACAAGCGTTAATGGTCACTATTGCCGGG | | |
| | | | ATTGCGGCAGGTATGCGTAATACCGGCTAA | | |
| pyc | Streptomyces | AL939105.1 | ATGGTCTCGTCACCCGGCAGGCTGAAGGGATCAAGAAT | 40 | |
| | coelicolor | | GTTCCGCAAGGTGCTGGTCGCCAACCGCGGTGAGATCG | | |
| | | | CGATCCGTGCGTTTCGGGCGGGCTACGAGCTCGGCGCG | | |
| | | | CGCACCGTCGCCGTCTTCCCGCACGAGGACCGCAATTC | | |
| | | | GCTGCACCGGCTCAAGGCCGACGAGGCCTACGAGATCG | | |
| | | | GGGAGCAGGGGCATCCCGTCCGCGCGTACCTCTCCGTG | | |
| | | | GAGGAGATCGTGCGCGCCGCCCGCCGTGCGGGGGCCGA | | |
| | | | CGCCGTCTACCCGGGCTACGGCTTCCTGTCCGAGAACC | | |
| | | | CCGAACTCGCCCGCGCCTGCGAGGAGGCCGGGATCACC | | |
| | | | TTCGTCGGTCCCAGCGCCCGGATCCTGGAACTGACCGG | | |
| | | | CAACAAGGCACGGGCCGTGGCCGCCGCCCGCGAGGCCG | | |
| | | | GAGTACCCGTGCTCGGCTCCTCGGCGCCCTCCACCGAC | | |
| | | | GTGGACGAACTCGTACGCGCCGCCGACGACGTCGGCTT | | |
| | | | CCCCGTGTTCGTCAAGGCGGTCGCGGGCGGCGGCGGGC | | |
| | | | GCGGCATGCGCCGCGTCGAGGAACCCGCCCAGCTGCGC | | |
| | | | GAGGCCATCGAGGCCGCCTCCCGCGAGGCCGCGTCCGC | | |
| | | | CTTCGGCGACTCCACCGTCTTCCTGGAGAAGGCGGTCG | | |
| | | | TCGAACCCCGCCACATCGAGGTGCAGATCCTCGCCGAC | | |
| | | | GGCGAGGGCGACGTCATCCACCTCTTCGAGCGGGACTG | | |
| | | | CTCGGTGCAGCGCCGCCACCAGAAGGTGATCGAGCTGG | | |
| | | | CGCCCGCGCCCAACCTCGACCCGGCCCTGCGGGAGCGG | | |
| | | | ATCTGCGCCGACGCCGTGAACTTCGCCCGGCAGATCGG | | |
| | | | CTACCGCAACGCGGGCACCGTCGAGTTCCTCGTCGACC | | |
| | | | GGGACGGCAACCACGTCTTCATCGAGATGAACCCGCGC | | |
| | | | ATCCAGGTCGAGCACACGGTCACCGAGGAGGTCACCGA | | |
| | | | CGTCGACCTGGTCCAGTCCCAGCTGCGCATCGCCGCCG | | |
| | | | GCCAGACGCTGGCCGACCTCGGACTCGCCCAGGAGAAC | | |
| | | | ATCACCCTGCGCGGTGCCGCACTCCAGTGCCGCATCAC | | |
| | | | CACCGAGGACCCGGCCAACGGCTTCCGCCCGGACACCG | | |
| | | | GGCAGATCAGCGCCTACCGTTCGCCGGGCGGCTCCGGC | | |
| | | | ATCCGGCTCGACGGCGGTACCACCCACGCCGGTACGGA | | |
| | | | GATCAGCGCGCACTTCGACTCGATGCTGGTCAAGCTCT | | |
| | | | CCTGCCGGGGACGGGACTTCACCACCGCGGTGAACCGC | | |
| | | | GCCCGGCGTGCGGTCGCCGAGTTCCGCATCCGCGGCGT | | |
| | | | CGCCACCAACATCCCCTTCCTCCAGGCGGTCCTGGACG | | |
| | | | ACCCCGACTTCCAGGCCGGCCGGGTCACCACCTCGTTC | | |
| | | | ATCGAACAGCGCCCGCACCTGCTGACCGCCCGGCACTC | | |
| | | | CGCCGACCGCGGCACCAAGCTGCTGACCTACCTCGCCG | | |
| | | | ACGTCACGGTGAACAAGCCGCACGGCGAGCGCCCCGAG | | |
| | | | CTGGTCGACCCGCTGACCAAGCTGCCGACGGCGTCCGC | | |
| | | | CGGTGAACCGCCCGCCGGGTCCCGCCAGTTGCTGGCCG | | |
| | | | AGCTGGGGCCGGAGGGGTTCGCCCGCCGACTGCGCGAG | | |
| | | | TCGTCCACCATCGGCGTCACCGACACCACCTTCCGCGA | | |
| | | | CGCCCACCAGTCGCTGCTCGCCACCCGGGTGCGCACCA | | |
| | | | AGGACATGCTCGCCGTGGCGCCCGTCGTCGCCCGCACC | | |
| | | | CTGCCCCAGCTGCTGTCCCTGGAGTGCTGGGGCGGCGC | | |
| | | | CACCTACGACGTCGCCCTGCGCTTCCTCGCCGAGGACC | | |
| | | | CCTGGGAGCGGCTAGCCGCGCTGCGCGAGGCGGTGCCC | | |
| | | | AACCTCTGCCTCCAGATGCTGCTGCGCGGCCGCAACAC | | |
| | | | CGTGGGCTACACCCCGTACCCGACCGAGGTGACCGACG | | |
| | | | CCTTCGTGCAGGAGGCCGCCGCCACCGGCATCGACATC | | |
| | | | TTCCGCATCTTCGACGCCCTCAACGACGTCGAGCAGAT | | |
| | | | GCGGCCCGCCATCGAGGCCGTACGGCAGACCGGCAGCG | | |
| | | | CCGTCGCCGAGGTCGCGCTCTGCTACACCGCCGACCTG | | |
| | | | TCCGACCCCTCCGAGCGGCTCTACACCCTCGACTACTA | | |
| | | | CCTGCGGCTCGCCGAGCAGATCGTGAACGCCGGAGCGC | | |
| | | | ACGTGCTGGCCGTCAAGGACATGGCCGGGCTGCTGCGC | | |
| | | | GCACCGGCCGCCGCGACCCTGGTGTCCGCGCTGCGCCG | | |
| | | | GGAGTTCGACCTGCCGGTGCACCTGCACACCCACGACA | | |
| | | | CCACCGGCGGCCAGCTCGCCACCTACCTGGCCGCGATC | | |
| | | | CAGGCGGGCGCGGACGCCGTCGACGGTGCGGTGGCGTC | | |
| | | | CATGGCGGGCACCACTTCGCAGCCGTCGCTGTCGGCGA | | |
| | | | TCGTGGCCGCCACCGACCACACCGAGCGGCCCACCGGC | | |
| | | | CTCGACCTCCAGGCCGTCGGCGACCTGGAGCCGTACTG | | |
| | | | GGAGAGCGTCCGCAAGGTCTACGCCCCGTTCGAGGCCG | | |
| | | | GCCTGGCCTCCCCGACCGGCCGGGTCTACCACCACGAG | | |
| | | | ATTCCCGGCGGCCAGCTCTCCAACCTGCGCACCCAGGC | | |
| | | | CGTCGCGCTCGGCCTCGGCGACCGCTTCGAGGACATCG | | |
| | | | AGGCCATGTACGCCGCCGCCGACCGGATGCTGGGCCGC | | |
| | | | CTGGTGAAGGTCACCCCGTCCTCCAAGGTGGTCGGCGA | | |
| | | | CCTGGCCCTGCATCTGGTGGGCGCCGGTGTCTCCCCGG | | |
| | | | CGGACTTCGAGCAGGACCCCGACCGGTTCGACATCCCG | | |
| | | | GACTCCGTGGTCGGCTTCCTGCGCGGCGAGCTGGGCAC | | |
| | | | CCCGCCCGGCGGCTGGCCCGAGCCGTTCCGCAGCAAGG | | |
| | | | CGCTGCGCGGCCGCGCCGAGGCCAGGCCGCTCGCCGAG | | |
| | | | CTGTCCGAGGACGACCGCGACGGCCTCGGCAAGGACCG | | |
| | | | CCGGGCGACGCTCAACCGGCTGCTGTTCCCGGGACCGG | | |
| | | | CCCGCGAGTTCGACACCCACCGCGCCTCGTACGGCGAC | | |
| | | | ACCAGCATCCTCGACAGCAAGGACTTCTTCTACGGGCT | | |
| | | | GCGCCCGGGCAAGGAGTACACGGTCGACCTCGACCCCG | | |
| | | | GCGTCCGGCTGCTCATCGAACTCCAGGCGGTCGGCGAC | | |
| | | | GCCGACGAGCGCGGCATGCGCACCGTGATGTCCTCCCT | | |
| | | | GAACGGACAGCTCCGCCCCATCCAGGTCCGCGACCGGT | | |
| | | | CGGCCGCCACCGACGTCCCGGTGACGGAGAAGGCCGAC | | |
| | | | CGGGCGAACCCCGGCCACGTCGCGGCGCCGTTCGCCGG | | |
| | | | TGTGGTGACCCTCGCCGTCGCCGAGGGCGACGAGGTGG | | |
| | | | AGGCCGGGGCCACCGTGGCCACCATCGAGGCGATGAAG | | |
| | | | ATGGAGGCGTCGATCACGGCCCCGAAGTCCGGCACGGT | | |
| | | | GACCAGGCTCGCCATCAACCGCATCCAGCAGGTCGAGG | | |
| | | | GCGGCGATCTTCTCGTCCAACTCGCC | | |
| pyc | Mycobacterium | AF262949 | GTGATCTCCAAGGTGCTCGTCGCCAACCGCGGCGAAAT | 41 | |
| | smegmatis | | CGCGATCCGCGCATTCCGTGCTGCGTACGAGATGGGCA | | |
| | | | TCGCCACGGTGGCGGTGTATCCGTACGAGGACCGGAAT | | |
| | | | TCGCTCCATCGGCTCAAGGCCGACGAGTCATATCAGAT | | |
| | | | CGGCGAGGTGGGTCATCCCGTCCGCGCGTATCTGTCGG | | |
| | | | TCGACGAGATCATCCGCGTCGCCAAGCATTCGGGCGCC | | |
| | | | GACGCGGTGTACCCGGGCTACGGCTTCCTGTCGGAGAA | | |
| | | | CCCCGATCTGGCGGCCAAGTGCGCCGAGGCGGGTATCA | | |
| | | | CGTTCGTGGGACCGTCCGCCGAGGTGCTGCAGCTCACG | | |
| | | | GGTAACAAGGCACGCGCGATCGCCGCGGCGCGCGCCGC | | |
| | | | GGGCCTTCCCGTGCTGAGTTCGTCGGAGCCGTCGTCGT | | |
| | | | CGGTGGACGAGTTGATGGCCGCTGCCGCCGACATGGAG | | |
| | | | TTCCCGCTGTTCGTCAAGGCGGTCTCGGGTGGCGGCGG | | |
| | | | GCGCGGCATGCGCCGCGTCACCGACCGCGAGTCCCTGG | | |
| | | | CCGAGGCGATCGAGGCGGCCTCGCGGGAGGCCGAGTCG | | |
| | | | GCGTTCGGCGACGCGTCGGTGTACCTGGAGCAGGCCGT | | |
| | | | GCTCAACCCGCGTCACATCGAGGTGCAGATCCTCGCCG | | |
| | | | ACGGCGCGGGCAACGTCATGCACCTGTTCGAGCGTGAC | | |
| | | | TGCAGCGTGCAGCGCAGGCATCAGAAGGTCGTCGAGCT | | |
| | | | GGCGCCCGCGCCCAACCTGAGTGACGAACTGCGCCAAC | | |
| | | | AGATCTGCGCCGACGCCGTGGCCTTCGCGCGCCAGATC | | |
| | | | GGGTACTCGTGCGCGGGCACCGTCGAGTTCCTGCTCGA | | |
| | | | CGAGCGCGGCCATCACGTGTTCATCGAGTGCAATCCGC | | |
| | | | GAATCCAGGTGGAGCACACGGTGACCGAGGAGATCACC | | |
| | | | GACGTGGACCTGGTGTCCTCGCAGTTGCGCATCGCCGC | | |
| | | | GGGCGAGACGCTCGCCGATCTCGGTCTGTCCCAGGACC | | |
| | | | GGCTCGTGGTGCGTGGCGCGGCCATGCAGTGCCGCATC | | |
| | | | ACCACCGAGGTCCCGGCCAACGGCTTCCGACCCGACAC | | |
| | | | CGGCCGCATCACCGCGTACCGCTCGCCGGGCGGCGCGG | | |
| | | | GCATCCGCCTCGACGGCGGCACCAACCTGGGTGCGGAG | | |
| | | | ATCTCGGCGCACTTCGACTCCATGCTGGTCAAGCTGAC | | |
| | | | GTGCCGGGGACGCGACTTCTCGGCCGCGGCCTCGCGCG | | |
| | | | CGCGCCGCGCCCTGGCGGAGTTCCGCATCCGCGGTGTG | | |
| | | | TCGACCAACATCCCGTTCCTGCAGGCGGTCATCGACGA | | |
| | | | TCCGGACTTCCGCGCCGGACGGGTGACGACGTCGTTCA | | |
| | | | TCGACGACCGGCCGCATCTATTGACCTCGCGGTCTCCT | | |
| | | | GCCGACCGCGGCACCAGGATCCTCAACTACCTGGCCGA | | |
| | | | CATCACGGTCAACAAGCCGCACGGCGAACGGCCTTCGA | | |
| | | | CGGTTTACCCGCAGGACAAGCTGCCGCCGCTGGATCTG | | |
| | | | CAGGCGCCGCCGCCCGCGGGATCCAAACAGCGCCTCGT | | |
| | | | GGAACTGGGGCCCGAGGGTTTCGCGGGCTGGCTGCGCG | | |
| | | | AATCCAAGGCCGTCGGCGTCACCGACACGACGTTCCGC | | |
| | | | GACGCGCACCAGTCGCTGCTGGCCACGCGTGTGCGCAC | | |
| | | | CACCGGTCTGCTGATGGTGGCGCCGTACGTCGCACGCT | | |
| | | | CCATGCCGCAGTTGCTGTCGATCGAGTGCTGGGGCGGC | | |
| | | | GCGACCTACGATGTGGCCCTTCGCTTCCTGAAGGAAGA | | |
| | | | CCCGTGGGAGCGGCTGGCGGCGCTGCGCGAGAGCGTGC | | |
| | | | CCAACATCTGCCTGCAGATGCTGCTGCGGGGACGCAAC | | |
| | | | ACCGTGGGCTACACGCCGTACCCGGAACTGGTCACCTC | | |
| | | | GGCGTTCGTCGAGGAGGCCGCGGCGACCGGTATCGACA | | |
| | | | TCTTCCGGATCTTCGACGCGCTCAACAACGTCGAGTCG | | |
| | | | ATGCGGCCCGCGATCGACGCGGTGCGGGAAACCGGTTC | | |
| | | | GACCATCGCCGAAGTCGCGATGTGCTACACGGGCGACC | | |
| | | | TCAGCGATCCCGCGGAGAACCTCTACACGCTCGACTAC | | |
| | | | TACCTGAAGCTGGCCGAGCAGATCGTCGAGGCCGGCGC | | |
| | | | CCACGTGCTGGCGATCAAGGACATGGCCGGTCTGCTGC | | |
| | | | GCGCCCCGGCCGCCCACACGCTCGTGAGCGCGTTGCGC | | |
| | | | AGCCGGTTCGATCTGCCCGTGCACGTGCACACCCACGA | | |
| | | | CACCCCGGGCGGTCAGCTCGCGACGTACCTCGCGGCGT | | |
| | | | GGTCGGCCGGCGCGGACGCGGTGGACGGCGCCTCGGCG | | |
| | | | CCGATGGCCGGGACCACGAGCCAGCCCGCGCTGAGCTC | | |
| | | | GATCGTCGCGGCGGCCGCGCACACCCAGTACGACACGG | | |
| | | | GCCTGGACCTGCGTGCGGTGTGCGACCTTGAGCCCTAC | | |
| | | | TGGGAGGCGGTGAGAAAGGTCTACGCGCCGTTCGAGTC | | |
| | | | CGGGCTGCCCGGGCCAACCGGCCGCGTCTACACCCACG | | |
| | | | AGATTCCCGGTGGGCAGTTGAGCAACCTGCGTCAGCAG | | |
| | | | GCCATCGCGTTGGGCCTCGGCGACCGGTTCGAGGAGAT | | |
| | | | CGAGGCCAATTACGCTGCGGCCGACCGGGTTCTGGGAC | | |
| | | | GGCTCGTGAAGGTGACCCCGTCGTCGAAGGTGGTCGGG | | |
| | | | GACCTGGCGCTGGCGCTCGTGGGTGCGGGCATCACCGC | | |
| | | | CGAGGAGTTCGCCGAGGATCCCGCGAAGTACGACATCC | | |
| | | | CCGACAGCGTGATCGGCTTCCTGCGCGGTGAACTCGGG | | |
| | | | GATCCGCCGGGCGGATGGCCGGAACCGTTGCGCACCAA | | |
| | | | GGCGCTCCAGGGCCGCGGACCGGCCCGGCCGGTCGAGA | | |
| | | | AGCTGACCGCCGACGACGAGGCGTTGCTCGCCCAGCCC | | |
| | | | GGGCCCAAGCGGCAGGCCGCGTTGAACCGCCTGCTTTT | | |
| | | | CCCCGGGCCCACCGCCGAGTTCGAGGCGCACCGCGAAA | | |
| | | | CCTACGGCGACACCTCATCCCTCAGCGCGAACCAGTTC | | |
| | | | TTCTACGGGCTGCGCTACGGCGAGGAGCACCGCGTGCA | | |
| | | | ACTCGAACGTGGCGTGGAACTGCTGATCGGGCTTGAGG | | |
| | | | CGATCTCGGAGGCCGACGAGCGCGGCATGCGCACCGTG | | |
| | | | ATGTGCATCATCAACGGTCAGCTGCGCCCGGTTCTCGT | | |
| | | | GCGCGACCGCAGCATCGCCAGCGAGGTGCCCGCCGCCG | | |
| | | | AAAAGGCCGACCGCAACAATGCCGACCACATCGCCGCG | | |
| | | | CCCTTCGCCGGTGTGGTGACCGTCGGTGTCGCAGAAGG | | |
| | | | TGACTCGGTGGACGCGGGACAAACCATCGCGACGATCG | | |
| | | | AGGCGATGAAGATGGAGGCCGCCATCACCGCGCCCAAG | | |
| | | | GCAGGCACCGTCGCGCGCGTCGCGGTCGCGGCGACCGC | | |
| | | | CCAGGTCGAGGGCGGCGATCTGCTGGTGGTGGTCAGCT | | |
| | | | GA | | |
| pyc | Coryne- | Y09548 | GTGTCGACTCACACATCTTCAACGCTTCCAGCATTCAA | 244 | |
| | bacterium | | AAAGATCTTGGTAGCAAACCGCGGCGAAATCGCGGTCC | | |
| | glutamicum | | GTGCTTTCCGTGCAGCACTCGAAACCGGTGCAGCCACG | | |
| | | | GTAGCTATTTACCCCCGTGAAGATCGGGGATCATTCCA | | |
| | | | CCGCTCTTTTGCTTCTGAAGCTGTCCGCATTGGTACCG | | |
| | | | AAGGCTCACCAGTCAAGGCGTACCTGGACATCGATGAA | | |
| | | | ATTATCGGTGCAGCTAAAAAAGTTAAAGCAGATGCCAT | | |
| | | | TTACCCGGGATACGGCTTCCTGTCTGAAAATGCCCAGC | | |
| | | | TTGCCCGCGAGTGTGCGGAAAACGGCATTACTTTTATT | | |
| | | | GGCCCAACCCCAGAGGTTCTTGATCTCACCGGTGATAA | | |
| | | | GTCTCGCGCGGTAACCGCCGCGAAGAAGGCTGGTCTGC | | |
| | | | CAGTTTTGGCGGAATCCACCCCGAGCAAAAACATCGAT | | |
| | | | GAGATCGTTAAAAGCGCTGAAGGCCAGACTTACCCCAT | | |
| | | | CTTTGTGAAGGCAGTTGCCGGTGGTGGCGGACGCGGTA | | |
| | | | TGCGTTTTGTTGCTTCACCTGATGAGCTTCGCAAATTA | | |
| | | | GCAACAGAAGCATCTCGTGAAGCTGAAGCGGCTTTCGG | | |
| | | | CGATGGCGCGGTATATGTCGAACGTGCTGTGATTAACC | | |
| | | | CTCAGCATATTGAAGTGCAGATCCTTGGCGATCACACT | | |
| | | | GGAGAAGTTGTACACCTTTATGAACGTGACTGCTCACT | | |
| | | | GCAGCGTCGTCACCAAAAAGTTGTCGAAATTGCGCCAG | | |
| | | | CACAGCATTTGGATCCAGAACTGCGTGATCGCATTTGT | | |
| | | | GCGGATGCAGTAAAGTTCTGCCGCTCCATTGGTTACCA | | |
| | | | GGGCGCGGGAACCGTGGAATTCTTGGTCGATGAAAAGG | | |
| | | | GCAACCACGTCTTCATCGAAATGAACCCACGTATCCAG | | |
| | | | GTTGAGCACACCGTGACTGAAGAAGTCACCGAGGTGGA | | |
| | | | CCTGGTGAAGGCGCAGATGCGCTTGGCTGCTGGTGCAA | | |
| | | | CCTTGAAGGAATTGGGTCTGACCCAAGATAAGATCAAG | | |
| | | | ACCCACGGTGCAGCACTGCAGTGCCGCATCACCACGGA | | |
| | | | AGATCCAAACAACGGCTTCCGCCCAGATACCGGAACTA | | |
| | | | TCACCGCGTACCGCTCACCAGGCGGAGCTGGCGTTCGT | | |
| | | | CTTGACGGTGCAGCTCAGCTCGGTGGCGAAATCACCGC | | |
| | | | ACACTTTGACTCCATGCTGGTGAAAATGACCTGCCGTG | | |
| | | | GTTCCGACTTTGAAACTGCTGTTGCTCGTGCACAGCGC | | |
| | | | GCGTTGGCTGAGTTCACCGTGTCTGGTGTTGCAACCAA | | |
| | | | CATTGGTTTCTTGCGTGCGTTGCTGCGGGAAGAGGACT | | |
| | | | TCACTTCCAAGCGCATCGCCACCGGATTCATTGCCGAT | | |
| | | | CACCCGCACCTCCTTCAGGCTCCACCTGCTGATGATGA | | |
| | | | GCAGGGACGCATCCTGGATTACTTGGCAGATGTCACCG | | |
| | | | TGAACAAGCCTCATGGTGTGCGTCCAAAGGATGTTGCA | | |
| | | | GCTCCTATCGATAAGCTGCCTAACATCAAGGATCTGCC | | |
| | | | ACTGCCACGCGGTTCCCGTGACCGCCTGAAGCAGCTTG | | |
| | | | GCCCAGCCGCGTTTGCTCGTGATCTCCGTGAGCAGGAC | | |
| | | | GCACTGGCAGTTACTGATACCACCTTCCGCGATGCACA | | |
| | | | CCAGTCTTTGCTTGCGACCCGAGTCCGCTCATTCGCAC | | |
| | | | TGAAGCCTGCGGCAGAGGCCGTCGCAAAGCTGACTCCT | | |
| | | | GAGCTTTTGTCCGTGGAGGCCTGGGGCGGCGCGACCTA | | |
| | | | CGATGTGGCGATGCGTTTCCTCTTTGAGGATCCGTGGG | | |
| | | | ACAGGCTCGACGAGCTGCGCGAGGCGATGCCGAATGTA | | |
| | | | AACATTCAGATGCTGCTTCGCGGCCGCAACACCGTGGG | | |
| | | | ATACACCCCGTACCCAGACTCCGTCTGCCGCGCGTTTG | | |
| | | | TTAAGGAAGCTGCCAGCTCCGGCGTGGACATCTTCCGC | | |
| | | | ATCTTCGACGCGCTTAACGACGTCTCCCAGATGCGTCC | | |
| | | | AGCAATCGACGCAGTCCTGGAGACCAACACCGCGGTAG | | |
| | | | CCGAGGTGGCTATGGCTTATTCTGGTGATCTCTCTGAT | | |
| | | | CCAAATGAAAAGCTCTACACCCTGGATTACTACCTAAA | | |
| | | | GATGGCAGAGGAGATCGTCAAGTCTGGCGCTCACATCT | | |
| | | | TGGCCATTAAGGATATGGCTGGTCTGCTTCGCCCAGCT | | |
| | | | GCGGTAACCAAGCTGGTCACCGCACTGCGCCGTGAATT | | |
| | | | CGATCTGCCAGTGCACGTGCACACCCACGACACTGCGG | | |
| | | | GTGGCCAGCTGGCAACCTACTTTGCTGCAGCTCAAGCT | | |
| | | | GGTGCAGATGCTGTTGACGGTGCTTCCGCACCACTGTC | | |
| | | | TGGCACCACCTCCCAGCCATCCCTGTCTGCCATTGTTG | | |
| | | | CTGCATTCGCGCACACCCGTCGCGATACCGGTTTGAGC | | |
| | | | CTCGAGGCTGTTTCTGACCTCGAGCCGTACTGGGAAGC | | |
| | | | AGTGCGCGGACTGTACCTGCCATTTGAGTCTGGAACCC | | |
| | | | CAGGCCCAACCGGTCGCGTCTACCGCCACGAAATCCCA | | |
| | | | GGCGGACAGTTGTCCAACCTGCGTGCACAGGCCACCGC | | |
| | | | ACTGGGCCTTGCGGATCGTTTCGAACTCATCGAAGACA | | |
| | | | ACTACGCAGCCGTTAATGAGATGCTGGGACGCCCAACC | | |
| | | | AAGGTCACCCCATCCTCCAAGGTTGTTGGCGACCTCGC | | |
| | | | ACTCCACCTCGTTGGTGCGGGTGTGGATCCAGCAGACT | | |
| | | | TTGCTGCCGATCCACAAAAGTACGACATCCCAGACTCT | | |
| | | | GTCATCGCGTTCCTGCGCGGCGAGCTTGGTAACCCTCC | | |
| | | | AGGTGGCTGGCCAGAGCCACTGCGCACCCGCGCACTGG | | |
| | | | AAGGCCGCTCCGAAGGCAAGGCACCTCTGACGGAAGTT | | |
| | | | CCTGAGGAAGAGCAGGCGCACCTCGACGCTGATGATTC | | |
| | | | CAAGGAACGTCGCAATAGCCTCAACCGCCTGCTGTTCC | | |
| | | | CGAAGCCAACCGAAGAGTTCCTCGAGCACCGTCGCCGC | | |
| | | | TTCGGCAACACCTCTGCGCTGGATGATCGTGAATTCTT | | |
| | | | CTACGGCCTGGTCGAAGGCCGCGAGACTTTGATCCGCC | | |
| | | | TGCCAGATGTGCGCACCCCACTGCTTGTTCGCCTGGAT | | |
| | | | GCGATCTCTGAGCCAGACGATAAGGGTATGCGCAATGT | | |
| | | | TGTGGCCAACGTCAACGGCCAGATCCGCCCAATGCGTG | | |
| | | | TGCGTGACCGCTCCGTTGAGTCTGTCACCGCAACCGCA | | |
| | | | GAAAAGGCAGATTCCTCCAACAAGGGCCATGTTGCTGC | | |
| | | | ACCATTCGCTGGTGTTGTCACCGTGACTGTTGCTGAAG | | |
| | | | GTGATGAGGTCAAGGCTGGAGATGCAGTCGCAATCATC | | |
| | | | GAGGCTATGAAGATGGAAGCAACAATCACTGCTTCTGT | | |
| | | | TGACGGCAAAATCGATCGCGTTGTGGTTCCTGCTGCAA | | |
| | | | CGAAGGTGGAAGGTGGCGACTTGATCGTCGTCGTTTCC | | |
| | | | TAA | | |
| dapA | Thermobifida | NZ_AAAQQ10 | ATGGTAGGCAGTACGACGCCGAACGCGCCCTTCGGCCA | 42 | |
| | fusca | 00040.1 | GATGTTGACCGCGATGATCACCCCCATGCTCGACAATG | | |
| | | | GGGAGGTGGACTACGACGGGGTGGCCCGCCTCGCGACC | | |
| | | | TACCTCGTCGATGAGCAGCGCAACGACGGCCTCATCGT | | |
| | | | CAACGGAACCACCGGAGAGTCCGCCACCACCAGCGATG | | |
| | | | AGGAGAAGGAGCGCATCCTCCGCACCGTGATCGACGCG | | |
| | | | GTCGGCGACCGCGCCACCATCGTTGCCGGAGCGGGCAG | | |
| | | | CAACGACACCAGGCACAGTATTGAACTCGCGCGGACCG | | |
| | | | CGGAACGCGCCGGAGCAGACGGCCTGCTGCTCGTCACC | | |
| | | | CCCTACTACAACCGGCCGCCCCAAGAAGGCCTGCTGCG | | |
| | | | GCACTTCACGGCCATTGCCGACGCCACAGGGCTGCCGA | | |
| | | | TCATGCTCTACGACATTCCTGGCCGCACAGGCACGCCG | | |
| | | | ATCGACTCCGAAACCCTGGTCCGGCTCGCCGAGCACCC | | |
| | | | CCGCATCGTCGCCAACAAGGACGCCAAAGACGACCTCG | | |
| | | | GCGCCAGCTCGTGGGTGATGTCCCGCACCGACCTCGCC | | |
| | | | TACTACAGCGGCAGCGACATGCTCAACCTGCCGCTGCT | | |
| | | | GTCCATCGGCGCCGCGGGCTTCGTCAGCGTGGTCGGCC | | |
| | | | ATGTCGTCGGCTCCGAACTGCACGACATGATCGACGCC | | |
| | | | TACCGGGCCGGGGACGTGGCCCGGGCTTTGGACATCCA | | |
| | | | CCGCCGCCTGATCCCCGTCTACCGGGGCATGTTCCGCA | | |
| | | | CCCAGGGAGTCATCACCACTAAGGCGGTGCTCGCCATG | | |
| | | | TTCGGGCTGCCCGCCGGAGTGGTCCGCGCCCCCCTGCT | | |
| | | | CGACGCGTCCCCCGAACTCAAAGAGCTGCTCCGCGAAG | | |
| | | | ACCTCGCCATGGCCGGGGTGAAGGGCCCCACTGGCCTT | | |
| | | | GCCTCCGCTCACGAGGACGCGGCCAGCGGGAGGGAAGC | | |
| | | | GGAACGACTCACGGAGGGGACCGCA | | |
| dapA | Mycobacterium | AL583922.1 | GTGACCACTGTCGGATTCGACGTCCCCGCACGTTTGGG | 43 | |
| | leprae (can be | | GACCCTGCTTACTGCGATGGTGACACCGTTTGACGCTG | | |
| | used to clone | | ATGGTTCTGTTGACACTGCGGCTGCGACGCGGCTGGCG | | |
| | M. smegmatis | | AACCGCCTGGTCGACGCGGGTTGTGATGGTCTGGTGCT | | |
| | gene) | | CTCGGGCACCACCGGCGAGTCGCCGACCACTACTGACG | | |
| | | | ACGAGAAACTCCAACTGTTGCGTGTCGTACTTGAGGCG | | |
| | | | GTAGGTGACCGAGCTAGAGTCATCGCCGGCGCAGGTAG | | |
| | | | TTATGACACAGCTCATAGTGTCCGACTCGTCAAGGCCT | | |
| | | | GTGCGGGTGAGGGCGCGCACGGACTTCTGGTGGTTACC | | |
| | | | CCTTACTACTCGAAGCCGCCGCAGACCGGGCTGTTTGC | | |
| | | | GCACTTCACCGCTGTGGCCGACGCGACTGAGCTACCAG | | |
| | | | TGTTGCTCTACGACATTCCCGGGCGGTCGGTCGTGCCG | | |
| | | | ATCGAGCCTGACACGATTCGCGCGCTGGCGTCGCATCC | | |
| | | | CAACATCGTCGGAGTCAAAGAGGCCAAGGCTGATTTAT | | |
| | | | ACAGCGGTGCCCGGATCATGGCTGACACCGGCCTGGCC | | |
| | | | TACTATTCCGGCGACGACGCACTGAACCTGCCCTGGCT | | |
| | | | GGCGGTGGGTGCCATCGGCTTCATCAGTGTGATTTCTC | | |
| | | | ATCTAGCCGCAGGACAGCTTCGAGAGCTGTTATCCGCT | | |
| | | | TTTGGTTCTGGGGATATTACCACTGCCCGAAAGATCAA | | |
| | | | CGTCGCGATCGGCCCGCTGTGCAGCGCGATGGACCGCT | | |
| | | | TGGGTGGGGTGACGATGTCCAAGGCAGGTCTGCGGCTT | | |
| | | | CAGGGTATCGACGTCGGTGATCCGCGGTTGCCGCAGAT | | |
| | | | GCCGGCAACAGCGGAGCAGATCGATGAGTTGGCTGTCG | | |
| | | | ATATGCGTGCAGCCTCGGTGCTTAGG | | |
| dapA | Mycobacterium | AL008967.1 | GTGACCACCGTCGGATTCGACGTCGCAGCGCGCCTAGG | 44 | |
| | tuberculosis | | AACCCTGCTGACCGCGATGGTGACACCGTTTAGCGGCG | | |
| | (can be used to | | ATGGCTCCCTGGACACCGCCACCGCGGCGCGGCTGGCC | | |
| | clone M. | | AACCACCTGGTCGATCAGGGGTGCGACGGTCTGGTGGT | | |
| | smegmatis | | CTCGGGCACCACCGGCGAGTCGCCGACCACCACCGACG | | |
| | gene) | | GGGAGAAAATCGAGCTGCTGCGGGCCGTCTTGGAAGCG | | |
| | | | GTGGGGGACCGGGCCCGTGTTATCGCCGGTGCCGGCAC | | |
| | | | CTATGACACCGCGCACAGCATCCGGCTGGCCAAGGCTT | | |
| | | | GTGCGGCCGAGGGTGCGCACGGGCTGCTGGTGGTCACG | | |
| | | | CCCTACTATTCCAAGCCGCCGCAGCGGGGGCTGCAAGC | | |
| | | | CCATTTCACCGCCGTCGCCGACGCGACCGAGCTGCCGA | | |
| | | | TGCTGCTCTATGACATCCCGGGGCGGTCGGCGGTGCCG | | |
| | | | ATCGAGCCCGACACGATCCGCGCGTTGGCGTCGCATCC | | |
| | | | GAACATCGTCGGAGTCAAGGACGCCAAAGCCGACCTGC | | |
| | | | ACAGCGGCGCCCAAATCATGGCCGACACCGGACTGGCC | | |
| | | | TACTATTCCGGCGACGACGCGCTCAACCTGCCCTGGCT | | |
| | | | GGCCATGGGCGCCACGGGCTTCATCAGCGTGATTGCCC | | |
| | | | ACCTGGCAGCCGGGCAGCTTCGAGAGTTGTTGTCCGCC | | |
| | | | TTCGGTTCTGGGGATATCGCCACCGCCCGCAAGATCAA | | |
| | | | CATTGCGGTCGCCCCGCTGTGCAACGCGATGAGCCGCC | | |
| | | | TGGGTGGGGTGACGTTGTCCAAGGCGGGCTTGCGGCTG | | |
| | | | CAGGGCATCGACGTCGGTGATCCCCGGCTGCCCCAGGT | | |
| | | | GGCCGCGACACCGGAGCAGATCGACGCGTTGGCCGCCG | | |
| | | | ACATGCGCGCGGCCTCGGTGCTTCGG | | |
| dapA | Streptomyces | AL939124.1 | ATGGCTCCGACCTCCACTCCGCAGACCCCCTTCGGGCG | 45 | |
| | coelicolor | | GGTCCTCACCGCCATGGTCACGCCCTTCACGGCGGACG | | |
| | | | GCGCACTCGACCTCGACGGCGCCCAGCGGCTCGCCGCC | | |
| | | | CACCTGGTGGACGCAGGCAACGACGGCCTGATCATCAA | | |
| | | | CGGCACCACCGGCGAGTCCCCGACCACCAGCGACGCGG | | |
| | | | AGAAAGCGGACCTCGTACGGGCCGTCGTGGAGGCGGTC | | |
| | | | GGCGACCGGGCGCACGTGGTGGCCGGAGTCGGCACCAA | | |
| | | | CAACACCCAGCACAGCATCGAGCTGGCCCGCGCCGCCG | | |
| | | | AGCGCGTCGGCGCCCACGGCCTGCTGCTCGTCACGCCG | | |
| | | | TACTACAACAAGCCCCCGCAGGAGGGCCTGTACCTGCA | | |
| | | | CTTCACGGCCATCGCCGACGCCGCCGGGCTGCCGGTCA | | |
| | | | TGCTCTACGACATCCCCGGCCGCAGCGGCGTCCCGATC | | |
| | | | AACACCGAGACCCTGGTCCGCCTCGCGGAGCACCCGCG | | |
| | | | GATCGTCGCCAACAAGGACGCCAAGGGCGACCTCGGCC | | |
| | | | GGGCCAGCTGGGCCATCGCGCGCTCCGGCCTCGCCTGG | | |
| | | | TACTCCGGCGACGACATGCTCAACCTGCCGCTGCTCGC | | |
| | | | CGTGGGCGCGGTCGGCTTCGTCTCCGTCGTGGGCCACG | | |
| | | | TCGTCACCCCGGAGCTGCGCGCCATGGTGGACGCGCAC | | |
| | | | GTCGCCGGTGACGTACAGAAGGCCCTGGAGATCCACCA | | |
| | | | GAAGCTGCTCCCCGTCTTCACCGGCATGTTCCGCACCC | | |
| | | | AGGGCGTCATGACCACCAAGGGCGCGCTCGCCCTCCAG | | |
| | | | GGACTGCCCGCGGGACCGCTGCGCGCCCCCATGGTCGG | | |
| | | | CCTCACGCCCGAGGAAACCGAGCAGCTCAAGATCGATC | | |
| | | | TTGCCGCCGGCGGGGTACAGCTC | | |
| dapA | Erwinia | | ATGTTTACGGGTAGTATTGTTGCTCTGGTTACGCCGAT | 46 | |
| | chrysanthemi | | GGACGACAAAGGTGCCGTTGATCGCGCGAGCTTGAAAA | | |
| | | | AACTGATTGATTATCATGTCGCTAGCGGAACTTCCGCG | | |
| | | | ATTGTGTCGGTGGGTACCACCGGCGAATCCGCCACCTT | | |
| | | | GAGTCACGATGAGCATGGCGACGTGGTGATGCTGACGC | | |
| | | | TGGAATTGAGCGATGGCCGCATCCCGGTCATCGCCGGC | | |
| | | | ACCGGCGCCAATTCGACCGCTGAGGCGATTTCCCTCAC | | |
| | | | CCAGCGTTTCAACGACACGGGCGTGGCCGGGTGCCTGA | | |
| | | | CCGTGACGCCGTATTACAATAAGCCGACCCAAAACGGC | | |
| | | | TTGTTCCTGCACTTCAAGGCGATTGCCGAGCACACCGA | | |
| | | | CCTGCCGCAAATCCTCTACAACGTGCCGTCCCGTACCG | | |
| | | | GTTGCGACATGTTGCCGGAAACCGTCGCCCGTCTGTCG | | |
| | | | GAAATCAAAAATATTGTCGCAATCAAGGAAGCGACCGG | | |
| | | | GAACTTAAGCCGGGTCAGTCAGATCCAAGAGCTGGTTC | | |
| | | | ATGAAGATTTCATTTTGCTGAGCGGCGACGACGCCAGC | | |
| | | | TCGCTGGACTTCATGCAACTGGGTGGCGACGGCGTGAT | | |
| | | | TTCCGTGACAGCCAACATCGCGGCCCGCGAAATGGCGG | | |
| | | | CGCTGTGCGAGCTGGCGGCGCAAGGGAATTTCGTTGAA | | |
| | | | GCCCGCCGTCTGAATCAGCGTCTGATGCCGCTGCATCA | | |
| | | | GAAACTGTTTGTTGAACCCAATCCGATTCCGGTGAAAT | | |
| | | | GGGCCTGTAAGGCATTGGGATTGATGGCGACCGACACG | | |
| | | | CTTCGTCTGCCGATGACGCCGCTGACCGATGCCGGTCG | | |
| | | | CGACGTGATGGAGCAGGCCATGAAGCAGGCGGGTCTGC | | |
| | | | TGTAA | | |
| dapA | Coryne- | X53993 | ATGAGCACAGGTTTAACAGCTAAGACCGGAGTAGAGCA | 128 | |
| | bacterium | | CTTCGGCACCGTTGGAGTAGCAATGGTTACTCCATTCA | | |
| | glutamicum | | CGGAATCCGGAGACATCGATATCGCTGCTGGCCGCGAA | | |
| | | | GTCGCGGCTTATTTGGTTGATAAGGGCTTGGATTCTTT | | |
| | | | GGTTCTCGCGGGCACCACTGGTGAATCCCCAACGACAA | | |
| | | | CCGCCGCTGAAAAACTAGAACTGCTCAAGGCCGTTCGT | | |
| | | | GAGGAAGTTGGGGATCGGGCGAAGCTCATCGCCGGTGT | | |
| | | | CGGAACCAACAACACGCGGACATCTGTGGAACTTGCGG | | |
| | | | AAGCTGCTGCTTCTGCTGGCGCAGACGGCCTTTTAGTT | | |
| | | | GTAACTCCTTATTACTCCAAGCCGAGCCAAGAGGGATT | | |
| | | | GCTGGCGCACTTCGGTGCAATTGCTGCAGCAACAGAGG | | |
| | | | TTCCAATTTGTCTCTATGACATTCCTGGTCGGTCAGGT | | |
| | | | ATTCCAATTGAGTCTGATACCATGAGACGCCTGAGTGA | | |
| | | | ATTACCTACGATTTTGGCGGTCAAGGACGCCAAGGGTG | | |
| | | | ACCTCGTTGCAGCCACGTCATTGATCAAAGAAACGGGA | | |
| | | | CTTGCCTGGTATTCAGGCGATGACCCACTAAACCTTGT | | |
| | | | TTGGCTTGCTTTGGGCGGATCAGGTTTCATTTCCGTAA | | |
| | | | TTGGACATGCAGCCCCCACAGCATTACGTGAGTTGTAC | | |
| | | | ACAAGCTTCGAGGAAGGCGACCTCGTCCGTGCGCGGGA | | |
| | | | AATCAACGCCAAACTATCACCGCTGGTAGCTGCCCAAG | | |
| | | | GTCGCTTGGGTGGAGTCAGCTTGGCAAAAGCTGCTTCG | | |
| | | | CGTCTGCAGGGCATCAACGTAGGAGATCCTCGACTTCC | | |
| | | | AATTATGGCTCCAAATGAGCAGGAACTTGAGGCTCTCC | | |
| | | | GAGAAGACATGAAAAAAGCTGGAGTTCTATAA | | |
| dapA | Escherichia | | ATGTTCACGGGAAGTATTGTCGCGATTGTTACTCCGAT | 129 | |
| | coli | | GGATGAAAAAGGTAATGTCTGTCGGGCTAGCTTGAAAA | | |
| | | | AACTGATTGATTATCATGTCGCCAGCGGTACTTCGGCG | | |
| | | | ATCGTTTCTGTTGGCACCACTGGCGAGTCCGCTACCTT | | |
| | | | AAATCATGACGAACATGCTGATGTGGTGATGATGACGC | | |
| | | | TGGATCTGGCTGATGGGCGCATTCCGGTAATTGCCGGG | | |
| | | | ACCGGCGCTAACGCTACTGCGGAAGCCATTAGCCTGAC | | |
| | | | GCAGCGCTTCAATGACAGTGGTATCGTCGGCTGCCTGA | | |
| | | | CGGTAACCCCTTACTACAATCGTCCGTCGCAAGAAGGT | | |
| | | | TTGTATCAGCATTTCAAAGCCATCGCTGAGCATACTGA | | |
| | | | CCTGCCGCAAATTCTGTATAATGTGCCGTCCCGTACTG | | |
| | | | GCTGCGATCTGCTCCCGGAAACGGTGGGCCGTCTGGCG | | |
| | | | AAAGTAAAAAATATTATCGGAATCAAAGAGGCAACAGG | | |
| | | | GAACTTAACGCGTGTAAACCAGATCAAAGAGCTGGTTT | | |
| | | | CAGATGATTTTGTTCTGCTGAGCGGCGATGATGCGAGC | | |
| | | | GCGCTGGACTTCATGCAATTGGGCGGTCATGGGGTTAT | | |
| | | | TTCCGTTACGACTAACGTCGCAGCGCGTGATATGGCCC | | |
| | | | AGATGTGCAAACTGGCAGCAGAAGAACATTTTGCCGAG | | |
| | | | GCACGCGTTATTAATCAGCGTCTGATGCCATTACACAA | | |
| | | | CAAACTATTTGTCGAACCCAATCCAATCCCGGTGAAAT | | |
| | | | GGGCATGTAAGGAACTGGGTCTTGTGGCGACCGATACG | | |
| | | | CTGCGCCTGCCAATGACACCAATCACCGACAGTGGTCG | | |
| | | | TGAGACGGTCAGAGCGGCGCTTAAGCATGCCGGTTTGC | | |
| | | | TGTAA | | |
| dapA | Coryne- | X53993 | ATGAGCACAGGTTTAACAGCTAAGACCGGAGTAGAGCA | 245 | |
| | bacterium | | CTTCGGCACCGTTGGAGTAGCAATGGTTACTCCATTCA | | |
| | glutamicum | | CGGAATCCGGAGACATCGATATCGCTGCTGGCCGCGAA | | |
| | | | GTCGCGGCTTATTTGGTTGATAAGGGCTTGGATTCTTT | | |
| | | | GGTTCTCGCGGGCACCACTGGTGAATCCCCAACGACAA | | |
| | | | CCGCCGCTGAAAAACTAGAACTGCTCAAGGCCGTTCGT | | |
| | | | GAGGAAGTTGGGGATCGGGCGAAGCTCATCGCCGGTGT | | |
| | | | CGGAACCAACAACACGCGGACATCTGTGGAACTTGCGG | | |
| | | | AAGCTGCTGCTTCTGCTGGCGCAGACGGCCTTTTAGTT | | |
| | | | GTAACTCCTTATTACTCCAAGCCGAGCCAAGAGGGATT | | |
| | | | GCTGGCGCACTTCGGTGCAATTGCTGCAGCAACAGAGG | | |
| | | | TTCCAATTTGTCTCTATGACATTCCTGGTCGGTCAGGT | | |
| | | | ATTCCAATTGAGTCTGATACCATGAGACGCCTGAGTGA | | |
| | | | ATTACCTACGATTTTGGCGGTCAAGGACGCCAAGGGTG | | |
| | | | ACCTCGTTGCAGCCACGTCATTGATCAAAGAAACGGGA | | |
| | | | CTTGCCTGGTATTCAGGCGATGACCCACTAAACCTTGT | | |
| | | | TTGGCTTGCTTTGGGCGGATCAGGTTTCATTTCCGTAA | | |
| | | | TTGGACATGCAGCCCCCACAGCATTACGTGAGTTGTAC | | |
| | | | ACAAGCTTCGAGGAAGGCGACCTCGTCCGTGCGCGGGA | | |
| | | | AATCAACGCCAAACTATCACCGCTGGTAGCTGCCCAAG | | |
| | | | GTCGCTTGGGTGGAGTCAGCTTGGCAAAAGCTGCTTCG | | |
| | | | CGTCTGCAGGGCATCAACGTAGGAGATCCTCGACTTCC | | |
| | | | AATTATGGCTCCAAATGAGCAGGAACTTGAGGCTCTCC | | |
| | | | GAGAAGACATGAAAAAAGCTGGAGTTCTATAA | | |
| dapA | Escherichia | M12844 | ATGTTCACGGGAAGTATTGTCGCGATTGTTACTCCGAT | 246 | |
| | coli | | GGATGAAAAAGGTAATGTCTGTCGGGCTAGCTTGAAAA | | |
| | | | AACTGATTGATTATCATGTCGCCAGCGGTACTTCGGCG | | |
| | | | ATCGTTTCTGTTGGCACCACTGGCGAGTCCGCTACCTT | | |
| | | | AAATCATGACGAACATGCTGATGTGGTGATGATGACGC | | |
| | | | TGGATCTGGCTGATGGGCGCATTCCGGTAATTGCCGGG | | |
| | | | ACCGGCGCTAACGCTACTGCGGAAGCCATTAGCCTGAC | | |
| | | | GCAGCGCTTCAATGACAGTGGTATCGTCGGCTGCCTGA | | |
| | | | CGGTAACCCCTTACTACAATCGTCCGTCGCAAGAAGGT | | |
| | | | TTGTATCAGCATTTCAAAGCCATCGCTGAGCATACTGA | | |
| | | | CCTGCCGCAAATTCTGTATAATGTGCCGTCCCGTACTG | | |
| | | | GCTGCGATCTGCTCCCGGAAACGGTGGGCCGTCTGGCG | | |
| | | | AAAGTAAAAAATATTATCGGAATCAAAGAGGCAACAGG | | |
| | | | GAACTTAACGCGTGTAAACCAGATCAAAGAGCTGGTTT | | |
| | | | CAGATGATTTTGTTCTGCTGAGCGGCGATGATGCGAGC | | |
| | | | GCGCTGGACTTCATGCAATTGGGCGGTCATGGGGTTAT | | |
| | | | TTCCGTTACGACTAACGTCGCAGCGCGTGATATGGCCC | | |
| | | | AGATGTGCAAACTGGCAGCAGAAGAACATTTTGCCGAG | | |
| | | | GCACGCGTTATTAATCAGCGTCTGATGCCATTACACAA | | |
| | | | CAAACTATTTGTCGAACCCAATCCAATCCCGGTGAAAT | | |
| | | | GGGCATGTAAGGAACTGGGTCTTGTGGCGACCGATACG | | |
| | | | CTGCGCCTGCCAATGACACCAATCACCGACAGTGGTCG | | |
| | | | TGAGACGGTCAGAGCGGCGCTTAAGCATGCCGGTTTGC | | |
| | | | TGTAA | | |
| hom | Streptomyces | AL939123.1 | ATGATGCGTACGCGTCCGCTGAAGGTGGCGCTGCTGGG | 47 | |
| | coelicolor | | CTGTGGAGTGGTCGGCTCAAAGGTGGCGCGCATCATGA | | |
| | | | CGACGCACGCCGCCGACCTCGCCGCCCGGATCGGGGCC | | |
| | | | CCGGTGGAGCTCGCGGGCGTCGCCGTACGGCGGCCCGA | | |
| | | | CAAGGTGCGGGAGGGGATCGACCCGGCCCTCGTCACCA | | |
| | | | CCGACGCCACCGCGCTCGTCAAGCGCGGGGACATCGAC | | |
| | | | GTCGTCGTCGAGGTCATCGGGGGGATCGAGCCCGCGCG | | |
| | | | GACGCTCATCACCACCGCCTTCGCGCACGGCGCCTCCG | | |
| | | | TGGTCTCCGCCAACAAGGCGCTCATCGCCCAGGACGGC | | |
| | | | GCCGCCCTGCACGCCGCCGCCGACGAGCACGGCAAGGA | | |
| | | | CCTGTACTACGAGGCCGCCGTCGCCGGTGCCATCCCGC | | |
| | | | TGATCCGGCCGCTGCGCGAGTCCCTCGCCGGCGACAAG | | |
| | | | GTCAACCGGGTGCTCGGCATCGTCAACGGGACCACCAA | | |
| | | | CTTCATCCTCGACGCCATGGACTCGACCGGGGCCGGCT | | |
| | | | ATCAGGAAGCGCTCGACGAGGCCACGGCCCTCGGGTAC | | |
| | | | GCCGAGGCCGACCCGACCGCCGACGTCGAGGGCTTCGA | | |
| | | | CGCCGCAGCCAAGGCCGCCATCCTCGCCGGGATCGCCT | | |
| | | | TCCACACGCGCGTACGCCTCGACGACGTCTACCGCGAG | | |
| | | | GGCATGACCGAGGTCACCGCCGCCGACTTCGCCTCCGC | | |
| | | | CAAGGAGATGGGCTGCACCATCAAGCTGCTCGCCATCT | | |
| | | | GCGAGCGGGCGGCGGACGGAGGGTCGGTCACCGCACGC | | |
| | | | GTGCATCCCGCGATGATCCCGCTCAGCCATCCGCTGGC | | |
| | | | CAACGTGCGCGAGGCGTACAACGCCGTGTTCGTGGAGT | | |
| | | | CCGACGCCGCCGGTCAGCTCATGTTCTACGGGCCCGGC | | |
| | | | GCCGGCGGTTCGCCGACCGCGTCCGCCGTGCTCGGCGA | | |
| | | | CCTGGTGGCCGTGTGCCGCAACCGGCTGGGCGGAGCGA | | |
| | | | CCGGACCCGGTGAGTCCGCGTACGCCGCCCTGCCCGTC | | |
| | | | TCCCCGATGGGCGACGTCGTCACGCGCTACCACATCAG | | |
| | | | CCTCGACGTGGCCGACAAACCGGGCGTGCTCGCCCAGG | | |
| | | | TCGCGACCGTGTTCGCGGAGCACGGTGTCTCCATCGAC | | |
| | | | ACCGTGCGGCAGTCCGGCAAGGACGGCGAGGCATCCCT | | |
| | | | CGTCGTCGTCACCCATCGCGCGTCCGACGCCGCCCTCG | | |
| | | | GCGGTACGGTCGAGGCGCTGCGCAAGCTCGACACCGTG | | |
| | | | CGGGGTGTCGCCAGCATCATGCGGGTTGAAGGAGAG | | |
| hom | Mycobacterium | AF126720 | ATGAGTAAGAAGCCCATCGGGGTAGCGGTACTGGGCCT | 48 | |
| | smegmatis | | GGGGAACGTCGGCAGCGAGGTCGTGCGCATCATCGCCG | | |
| | | | ACAGCGCGGACGATCTCGCGGCGCGCATCGGTGCGCCG | | |
| | | | CTGGAACTGCGCGGCGTCGGCGTGCGCCGTGTGGCCGA | | |
| | | | CGACCGCGGCGTGCCCACGGAACTGCTCACCGACGACA | | |
| | | | TCGACGCGCTGGTGTCGCGTGACGACGTCGACATCGTC | | |
| | | | GTCGAGGTCATGGGCCCCGTCGAACCGGCACGCAAGGC | | |
| | | | CATCCTGTCGGCGCTGGAGCAGGGCAAGTCGGTGGTCA | | |
| | | | CCGCCAACAAGGCGCTGATGGCCATGTCCACCGGCGAG | | |
| | | | CTCGCCCAGGCCGCCGAGAAGGCCCACGTGGACCTGTA | | |
| | | | TTTCGAGGCCGCAGTGGCCGGCGCCATCCCGGTGATCC | | |
| | | | GCCCGCTGACCCAGTCGCTGGCCGGTGACACGGTGCGC | | |
| | | | CGCGTGGCCGGCATCGTCAACGGCACCACCAACTACAT | | |
| | | | CCTGTCCGAGATGGACAGCACCGGCGCCGATTACACCA | | |
| | | | GCGCGCTGGCCGATGCGAGCGCCCTCGGTTACGCCGAG | | |
| | | | GCCGATCCCACCGCCGACGTCGAGGGCTACGACGCCGC | | |
| | | | GGCCAAGGCCGCGATCCTCGCTTCGATCGCGTTCCACA | | |
| | | | CCCGTGTGACCGCCGACGACGTGTACCGCGAGGGCATC | | |
| | | | ACCACGGTCAGCGCCGAGGACTTCGCGTCGGCACGCGC | | |
| | | | GCTGGGCTGCACCATCAAACTGCTCGCGATCTGCGAGC | | |
| | | | GGCTCACCTCCGACGAGGGCAAGGACCGGGTCTCGGCC | | |
| | | | CGCGTCTACCCGGCGCTCGTCCCGCTGACCCACCCGCT | | |
| | | | GGCCGCGGTCAACGGTGCGTTCAACGCGGTGGTGGTGG | | |
| | | | AAGCCGAGGCGGCCGGGCGGCTCATGTTCTACGGTCAA | | |
| | | | GGCGCCGGCGGTGCCCCCACCGCCTTTGCGGTGATGGG | | |
| | | | AGACGTGGTCATGGCGGCTCGCAACCGTGTCCAGGGCG | | |
| | | | GCCGTGGCCCGCGCGAATCGAAGTACGCCAAGCTGCCG | | |
| | | | ATCGCGCCCATCGGGTTCATCCCGACGCGCTACTACGT | | |
| | | | CAACATGAACGTGGCCGACCGGCCCGGCGTGTTGTCCG | | |
| | | | CTGTGGCAGCCGAATTC | | |
| hom | Thermobifida | NZ_AAAQ010 | ATGCGCCGCCCAGAACCTGCCGGTGCCGCGGATCGCGG | 49 | |
| | fusca | 00037.1 | TCGAACCCGGCCGCGCCATCGCCGGACCGGCGGGCATC | | |
| | | | ACCCTCTACGAGGTCGGCACGGTCAAGGACGTGGAGGG | | |
| | | | GATCCGCACCTATGTCAGTGTCGACGGCGGTATGAGCG | | |
| | | | ACAACATCCGCACCGCGCTGTACGGTGCGGAGTACACC | | |
| | | | TGTGTGCTGGCCTCGCGGCACAGCGACGCCGAGCCGAT | | |
| | | | GCTGTCCCGCCTGGTCGGCAAGCACTGCGAGAGCGGCG | | |
| | | | ACATCGTCGTGCGCGACCTCTACCTCCCTGCCGACCTG | | |
| | | | CGTCCCGGCGACCTGGTAGCAGTGGCCGCCACCGGCGC | | |
| | | | CTACTGCTACTCCATGGCCAGCAACTACAACCACGTGC | | |
| | | | CCCGGCCTGCCGTGGTCGCGGTCCGCGAGAAGAACGCC | | |
| | | | CGCGTCCTGGTGCGACGGGAAACCGAAGAAGACCTGTT | | |
| | | | GCGGCTGGACGTAGGCTGAGCAGTGGCCGACGACGCTC | | |
| | | | TGGCCACCACGACGAGGTTCTGGATACGGACAATGAAC | | |
| | | | GACGAAACGGGAGTCACCCCCTCATGGCACTGAAGGTG | | |
| | | | GCGCTGCTGGGTTGCGGCGTTGTGGGTTCTCAGGTGGT | | |
| | | | CCGGCTGCTCAACGAGCAGTCGCGTGAACTTGCGGAGC | | |
| | | | GCATCGGAACGCCCCTGGAGATCGGAGGCATCGCGGTG | | |
| | | | CGCCGCCTGGACCGCGCCCGGGGGACGGGCGTGGACCC | | |
| | | | CGACCTCCTCACCACCGACGCCATGGGTCTTGTGACCA | | |
| | | | GAGACGACATCGACCTCGTGGTGGAGGTCATCGGCGGC | | |
| | | | ATCGAGCCCGCCCGGTCGCTCATCCTGGCCGCGATCCA | | |
| | | | GAAGGGCAAGTCTGTGGTGACCGCCAACAAGGCGCTGC | | |
| | | | TCGCCGAGGACGGCGCGACCATCCACGCCGCTGCCCGG | | |
| | | | GAAGCGGGAGTTGACGTGTACTACGAGGCCAGCGTCGC | | |
| | | | CGGGGCCATCCCGCTGCTGCGGCCGCTGCGTGACTCCC | | |
| | | | TGGCCGGGGACCGCGTCAACCGGGTCTTGGGCATCGTC | | |
| | | | AACGGCACCACCAACTACATCCTGGACCGGATGGACAG | | |
| | | | CCTGGGCGCCGGCTTCACCGAGTCACTGGAGGAAGCCC | | |
| | | | AGGCCCTGGGATACGCCGAAGCCGACCCGACCGCCGAC | | |
| | | | GTGGAGGGCTTCGACGCCGCCGCTAAAGCCGCGATCCT | | |
| | | | GGCCCGGCTCGCCTTCCACACACCGGTGACCGCTGCCG | | |
| | | | ATGTGCACCGCGAAGGCATCACCGAGGTCTCCGCGGCC | | |
| | | | GACATCGCCAGCGCCAAGGCCATGGGCTGCGTGGTGAA | | |
| | | | ACTCCTCGCGATCTGCCAGCGCTCCGACGACGGCTCCA | | |
| | | | GCATCGGCGTGCGCGTCCACCCGGTGATGCTGCCCCGC | | |
| | | | GAACACCCGCTCGCCAGCGTCAAAGGCGCCTACAACGC | | |
| | | | GGTGTTCGTGGAAGCCGAGTCCGCCGGGCAGCTCATGT | | |
| | | | TCTACGGCGCGGGCGCGGGAGGCGTCCCCACCGCCAGC | | |
| | | | GCAGTCCTCGGCGACCTGGTCGCGGTGGCACGGAACCG | | |
| | | | CCTGGCCCGCACTTTCGTGGCCGACGGCCGGGCCGACG | | |
| | | | CGAAACTGCCCGTCCACCCCATGGGGGAGACCATCACC | | |
| | | | AGCTACCACGTGGCGCTGGACGTTGCCGACCGGCCCGG | | |
| | | | CGTGCTCGCCGGGGTCGCCAAAGTCTTCGCGGCCAACG | | |
| | | | GCGTGTCGATCAAGCACGTCCGCCAGGAAGGCCGCGGG | | |
| | | | GACGACGCCCAGCTCGTCCTGGTCAGCCACACCGCGCC | | |
| | | | GGATGCCGCCCTGGCCCGGACCGTGGAGCAACTGCGCA | | |
| | | | ACCACGAGGACGTCCGCGCGGTCGCCAGCGTGATGCGG | | |
| | | | GTCGAAACCTTCGACAACGAACGA | | |
| hom | Coryne- | Y00546 | ATGACCTCAGCATCTGCCCCAAGCTTTAACCCCGGCAA | 247 | |
| | bacterium | | GGGTCCCGGCTCAGCAGTCGGAATTGCCCTTTTAGGAT | | |
| | glutamicum | | TCGGAACAGTCGGCACTGAGGTGATGCGTCTGATGACC | | |
| | | | GAGTACGGTGATGAACTTGCGCACCGCATTGGTGGCCC | | |
| | | | ACTGGAGGTTCGTGGCATTGCTGTTTCTGATATCTCAA | | |
| | | | AGCCACGTGAAGGCGTTGCACCTGAGCTGCTCACTGAG | | |
| | | | GACGCTTTTGCACTCATCGAGCGCGAGGATGTTGACAT | | |
| | | | CGTCGTTGAGGTTATCGGCGGCATTGAGTACCCACGTG | | |
| | | | AGGTAGTTCTCGCAGCTCTGAAGGCCGGCAAGTCTGTT | | |
| | | | GTTACCGCCAATAAGGCTCTTGTTGCAGCTCACTCTGC | | |
| | | | TGAGCTTGCTGATGCAGCGGAAGCCGCAAACGTTGACC | | |
| | | | TGTACTTCGAGGCTGCTGTTGCAGGCGCAATTCCAGTG | | |
| | | | GTTGGCCCACTGCGTCGCTCCCTGGCTGGCGATCAGAT | | |
| | | | CCAGTCTGTGATGGGCATCGTTAACGGCACCACCAACT | | |
| | | | TCATCTTGGACGCCATGGATTCCACCGGCGCTGACTAT | | |
| | | | GCAGATTCTTTGGCTGAGGCAACTCGTTTGGGTTACGC | | |
| | | | CGAAGCTGATCCAACTGCAGACGTCGAAGGCCATGACG | | |
| | | | CCGCATCCAAGGCTGCAATTTTGGCATCCATCGCTTTC | | |
| | | | CACACCCGTGTTACCGCGGATGATGTGTACTGCGAAGG | | |
| | | | TATCAGCAACATCAGCGCTGCCGACATTGAGGCAGCAC | | |
| | | | AGCAGGCAGGCCACACCATCAAGTTGTTGGCCATCTGT | | |
| | | | GAGAAGTTCACCAACAAGGAAGGAAAGTCGGCTATTTC | | |
| | | | TGCTCGCGTGCACCCGACTCTATTACCTGTGTCCCACC | | |
| | | | CACTGGCGTCGGTAAACAAGTCCTTTAATGCAATCTTT | | |
| | | | GTTGAAGCAGAAGCAGCTGGTCGCCTGATGTTCTACGG | | |
| | | | AAACGGTGCAGGTGGCGCGCCAACCGCGTCTGCTGTGC | | |
| | | | TTGGCGACGTCGTTGGTGCCGCACGAAACAAGGTGCAC | | |
| | | | GGTGGCCGTGCTCCAGGTGAGTCCACCTACGCTAACCT | | |
| | | | GCCGATCGCTGATTTCGGTGAGACCACCACTCGTTACC | | |
| | | | ACCTCGACATGGATGTGGAAGATCGCGTGGGGGTTTTG | | |
| | | | GCTGAATTGGCTAGCCTGTTCTCTGAGCAAGGAATCTC | | |
| | | | CCTGCGTACAATCCGACAGGAAGAGCGCGATGATGATG | | |
| | | | CACGTCTGATCGTGGTCACCCACTCTGCGCTGGAATCT | | |
| | | | GATCTTTCCCGCACCGTTGAACTGCTGAAGGCTAAGCC | | |
| | | | TGTTGTTAAGGCAATCAACAGTGTGATCCGCCTCGAAA | | |
| | | | GGGACTAA | | |
| metL | Escherichia | V00305 | AGTGTGATTGCGCAGGCAGGGGCGAAAGGTCGTCAGCT | 248 | |
| | coli | | GCATAAATTTGGTGGCAGTAGTCTGGCTGATGTGAAGT | | |
| | | | GTTATTTGCGTGTCGCGGGCATTATGGCGGAGTACTCT | | |
| | | | CAGCCTGACGATATGATGGTGGTTTCCGCCGCCGGTAG | | |
| | | | CACCACTAACCGGTTGATTAGCTGGTTGAAACTAAGCC | | |
| | | | AGACCGATCGTCTCTCTGCGCATCAGGTTCAACAAACG | | |
| | | | CTGCGTCGCTATCAGTGCGATCTGATTAGCGGTCTGCT | | |
| | | | ACCCGCTGAAGAAGCCGATAGCCTCATTAGCGCTTTTG | | |
| | | | TCAGCGACCTTGAGCGCCTGGCGGCGCTGCTCGACAGC | | |
| | | | GGTATTAACGACGCAGTGTATGCGGAAGTGGTGGGCCA | | |
| | | | CGGGGAAGTATGGTCGGCACGTCTGATGTCTGCGGTAC | | |
| | | | TTAATCAACAAGGGCTGCCAGCGGCCTGGCTTGATGCC | | |
| | | | CGCGAGTTTTTACGCGCTGAACGCGCCGCACAACCGCA | | |
| | | | GGTTGATGAAGGGCTTTCTTACCCGTTGCTGCAACAGC | | |
| | | | TGCTGGTGCAACATCCGGGCAAACGTCTGGTGGTGACC | | |
| | | | GGATTTATCAGCCGCAACAACGCCGGTGAAACGGTGCT | | |
| | | | GCTGGGGCGTAACGGTTCCGACTATTCCGCGACACAAA | | |
| | | | TCGGTGCGCTGGCGGGTGTTTCTCGCGTAACCATCTGG | | |
| | | | AGCGACGTCGCCGGGGTATACAGTGCCGACCCGCGTAA | | |
| | | | AGTGAAAGATGCCTGCCTGCTGCCGTTGCTGCGTCTGG | | |
| | | | ATGAGGCCAGCGAACTGGCGCGCCTGGCGGCTCCCGTT | | |
| | | | CTTCACGCCCGTACTTTACAGCCGGTTTCTGGCAGCGA | | |
| | | | AATCGACCTGCAACTGCGCTGTAGCTACACGCCGGATC | | |
| | | | AAGGTTCCACGCGCATTGAACGCGTGCTGGCCTCCGGT | | |
| | | | ACTGGTGCGCGTATTGTCACCAGCCACGATGATGTCTG | | |
| | | | TTTGATTGAGTTTCAGGTGCCCGCCAGTCAGGATTTCA | | |
| | | | AACTGGGGCATAAAGAGATCGACCAAATCCTGAAACGC | | |
| | | | GCGCAGGTACGCCCGCTGGCGGTTGGCGTACATAACGA | | |
| | | | TCGCCAGTTGCTGCAATTTTGCTACACCTCAGAAGTGG | | |
| | | | CCGACAGTGCGCTGAAAATCCTCGACGAAGCGGGATTA | | |
| | | | CCTGGCGAACTGCGCCTGCGTCAGGGGCTGGCGCTGGT | | |
| | | | GGCGATGGTCGGTGCAGGCGTCACCCGTAACCCGCTGC | | |
| | | | ATTGCCACCGCTTCTGGCAGCAACTGAAAGGCCAGCCG | | |
| | | | GTCGAATTTACCTGGCAGTCCGATGACGGCATCAGCCT | | |
| | | | GGTGGCAGTACTGCGCACCGGCCCGACCGAAAGCCTGA | | |
| | | | TTCAGGGGCTGCATCAGTCCGTCTTCCGCGCAGAAAAA | | |
| | | | CGCATCGGCCTGGTATTGTTCGGTAAGGGCAATATCGG | | |
| | | | TTCCCGTTGGCTGGAACTGTTCGCCCGTGAGCAGAGCA | | |
| | | | CGCTTTCGGCACGTACCGGCTTTGAGTTTGTGCTGGCA | | |
| | | | GGTGTGGTGGACAGCCGCCGCAGCCTGTTGAGCTATGA | | |
| | | | CGGGCTGGACGCCAGCCGCGCGTTAGCCTTCTTCAACG | | |
| | | | ATGAAGCGGTTGAGCAGGATGAAGAGTCGTTGTTCCTG | | |
| | | | TGGATGCGCGCCCATCCGTATGATGATTTAGTGGTGCT | | |
| | | | GGACGTTACCGCCAGCCAGCAGCTTGCTGATCAGTATC | | |
| | | | TTGATTTCGCCAGCCACGGTTTCCACGTTATCAGCGCC | | |
| | | | AACAAACTGGCGGGAGCCAGCGACAGCAATAAATATCG | | |
| | | | CCAGATCCACGACGCCTTCGAAAAAACCGGGCGTCACT | | |
| | | | GGCTGTACAATGCCACCGTCGGTGCGGGCTTGCCGATC | | |
| | | | AACCACACCGTGCGCGATCTGATCGACAGCGGCGATAC | | |
| | | | TATTTTGTCGATCAGCGGGATCTTCTCCGGCACGCTCT | | |
| | | | CCTGGCTGTTCCTGCAATTCGACGGTAGCGTGCCGTTT | | |
| | | | ACCGAGCTGGTGGATCAGGCGTGGCAGCAGGGCTTAAC | | |
| | | | CGAACCTGACCCGCGTGACGATCTCTCTGGCAAAGACG | | |
| | | | TGAGTCGCAAGCTGGTGATTCTGGCGCGTGAAGCAGGT | | |
| | | | TACAACATCGAACCGGATCAGGTACGTGTGGAATCGCT | | |
| | | | GGTGCCTGCTCATTGCGAAGGCGGCAGCATCGACCATT | | |
| | | | TCTTTGAAAATGGCGATGAACTGAACGAGCAGATGGTG | | |
| | | | CAACGGCTGGAAGCGGCCCGCGAAATGGGGCTGGTGCT | | |
| | | | GCGCTACGTGGCGCGTTTCGATGCCAACGGTAAAGCGC | | |
| | | | GTGTAGGCGTGGAAGCGGTGCGTGAAGATCATCCGTTG | | |
| | | | CGATCACTGCTGCCGTGCGATAACGTCTTTGCCATCGA | | |
| | | | AAGCCGCTGGTATCGCGATAACCCTCTGGTGATCCGCG | | |
| | | | GACCTGGCGCTGGGCGCGACGTCACCGCCGGGGCGATT | | |
| | | | CAGTCGGATATCAACCGGCTGGCACAGTTGTTGTAA | | |
| thrA | Escherichia | U14003 | ATGCGAGTGTTGAAGTTCGGCGGTACATCAGTGGCAAA | 249 | |
| | coli | | TGCAGAACGTTTTCTGCGTGTTGCCGATATTCTGGAAA | | |
| | | | GCAATGCCAGGCAGGGGCAGGTGGCCACCGTCCTCTCT | | |
| | | | GCCCCCGCCAAAATCACCAACCACCTGGTGGCGATGAT | | |
| | | | TGAAAAAACCATTAGCGGCCAGGATGCTTTACCCAATA | | |
| | | | TCAGCGATGCCGAACGTATTTTTGCCGAACTTTTGACG | | |
| | | | GGACTCGCCGCCGCCCAGCCGGGGTTCCCGCTGGCGCA | | |
| | | | ATTGAAAACTTTCGTCGATCAGGAATTTGCCCAAATAA | | |
| | | | AACATGTCCTGCATGGCATTAGTTTGTTGGGGCAGTGC | | |
| | | | CCGGATAGCATCAACGCTGCGCTGATTTGCCGTGGCGA | | |
| | | | GAAAATGTCGATCGCCATTATGGCCGGCGTATTAGAAG | | |
| | | | CGCGCGGTCACAACGTTACTGTTATCGATCCGGTCGAA | | |
| | | | AAACTGCTGGCAGTGGGGCATTACCTCGAATCTACCGT | | |
| | | | CGATATTGCTGAGTCCACCCGCCGTATTGCGGCAAGCC | | |
| | | | GCATTCCGGCTGATCACATGGTGCTGATGGCAGGTTTC | | |
| | | | ACCGCCGGTAATGAAAAAGGCGAACTGGTGGTGCTTGG | | |
| | | | ACGCAACGGTTCCGACTACTCTGCTGCGGTGCTGGCTG | | |
| | | | CCTGTTTACGCGCCGATTGTTGCGAGATTTGGACGGAC | | |
| | | | GTTGACGGGGTCTATACCTGCGACCCGCGTCAGGTGCC | | |
| | | | CGATGCGAGGTTGTTGAAGTCGATGTCCTACCAGGAAG | | |
| | | | CGATGGAGCTTTCCTACTTCGGCGCTAAAGTTCTTCAC | | |
| | | | CCCCGCACCATTACCCCCATCGCCCAGTTCCAGATCCC | | |
| | | | TTGCCTGATTAAAAATACCGGAAATCCTCAAGCACCAG | | |
| | | | GTACGCTCATTGGTGCCAGCCGTGATGAAGACGAATTA | | |
| | | | CCGGTCAAGGGCATTTCCAATCTGAATAACATGGCAAT | | |
| | | | GTTCAGCGTTTCTGGTCCGGGGATGAAAGGGATGGTCG | | |
| | | | GCATGGCGGCGCGCGTCTTTGCAGCGATGTCACGCGCC | | |
| | | | CGTATTTCCGTGGTGCTGATTACGCAATCATCTTCCGA | | |
| | | | ATACAGCATCAGTTTCTGCGTTCCACAAAGCGACTGTG | | |
| | | | TGCGAGCTGAACGGGCAATGCAGGAAGAGTTCTACCTG | | |
| | | | GAACTGAAAGAAGGCTTACTGGAGCCGCTGGCAGTGAC | | |
| | | | GGAACGGCTGGCCATTATCTCGGTGGTAGGTGATGGTA | | |
| | | | TGCGCACCTTGCGTGGGATCTCGGCGAAATTCTTTGCC | | |
| | | | GCACTGGCCCGCGCCAATATCAACATTGTCGCCATTGC | | |
| | | | TCAGGGATCTTCTGAACGCTCAATCTCTGTCGTGGTAA | | |
| | | | ATAACGATGATGCGACCACTGGCGTGCGCGTTACTCAT | | |
| | | | CAGATGCTGTTCAATACCGATCAGGTTATCGAAGTGTT | | |
| | | | TGTGATTGGCGTCGGTGGCGTTGGCGGTGCGCTGCTGG | | |
| | | | AGCAACTGAAGCGTCAGCAAAGCTGGCTGAAGAATAAA | | |
| | | | CATATCGACTTACGTGTCTGCGGTGTTGCCAACTCGAA | | |
| | | | GGCTCTGCTCACCAATGTACATGGCCTTAATCTGGAAA | | |
| | | | ACTGGCAGGAAGAACTGGCGCAAGCCAAAGAGCCGTTT | | |
| | | | AATCTCGGGCGCTTAATTCGCCTCGTGAAAGAATATCA | | |
| | | | TCTGCTGAACCCGGTCATTGTTGACTGCACTTCCAGCC | | |
| | | | AGGCAGTGGCGGATCAATATGCCGACTTCCTGCGCGAA | | |
| | | | GGTTTCCACGTTGTCACGCCGAACAAAAAGGCCAACAC | | |
| | | | CTCGTCGATGGATTACTACCATCAGTTGCGTTATGCGG | | |
| | | | CGGAAAAATCGCGGCGTAAATTCCTCTATGACACCAAC | | |
| | | | GTTGGGGCTGGATTACCGGTTATTGAGAACCTGCAAAA | | |
| | | | TCTGCTCAATGCAGGTGATGAATTGATGAAGTTCTCCG | | |
| | | | GCATTCTTTCTGGTTCGCTTTCTTATATCTTCGGCAAG | | |
| | | | TTAGACGAAGGCATGAGTTTCTCCGAGGCGACCACGCT | | |
| | | | GGCGCGGGAAATGGGTTATACCGAACCGGACCCGCGAG | | |
| | | | ATGATCTTTCTGGTATGGATGTGGCGCGTAAACTATTG | | |
| | | | ATTCTCGCTCGTGAAACGGGACGTGAACTGGAGCTGGC | | |
| | | | GGATATTGAAATTGAACCTGTGCTGCCCGCAGAGTTTA | | |
| | | | ACGCCGAGGGTGATGTTGCCGCTTTTATGGCGAATCTG | | |
| | | | TCACAACTCGACGATCTCTTTGCCGCGCGCGTGGCGAA | | |
| | | | GGCCCGTGATGAAGGAAAAGTTTTGCGCTATGTTGGCA | | |
| | | | ATATTGATGAAGATGGCGTCTGCCGCGTGAAGATTGCC | | |
| | | | GAAGTGGATGGTAATGATCCGCTGTTCAAAGTGAAAAA | | |
| | | | TGGCGAAAACGCCCTGGCCTTCTATAGCCACTATTATC | | |
| | | | AGCCGCTGCCGTTGGTACTGCGCGGATATGGTGCGGGC | | |
| | | | AATGACGTTACAGCTGCCGGTGTCTTTGCTGATCTGCT | | |
| | | | ACGTACCCTCTCATGGAAGTTAGGAGTCTGA | | |
| metA | Mycobacterium | AL021841.1 | ATGACGATCTCCGATGTACCCACCCAGACGCTGCCCGC | 50 | |
| | tuberculosis | | CGAAGGCGAAATCGGCCTGATAGACGTCGGCTCGCTGC | | |
| | (can be used to | | AACTGGAAAGCGGGGCGGTGATCGACGATGTCTGTATC | | |
| | clone M. | | GCCGTGCAACGCTGGGGCAAATTGTCGCCCGCACGGGA | | |
| | smegmatis | | CAACGTGGTGGTGGTCTTGCACGCGCTCACCGGCGACT | | |
| | gene) | | CGCACATCACTGGACCCGCCGGACCCGGCCACCCCACC | | |
| | | | CCCGGCTGGTGGGACGGGGTGGCCGGGCCGGGTGCGCC | | |
| | | | GATTGACACCACCCGCTGGTGCGCGGTAGCTACCAATG | | |
| | | | TGCTCGGCGGCTGCCGCGGCTCCACCGGGCCCAGCTCG | | |
| | | | CTTGCCCGCGACGGAAAGCCTTGGGGCTCAAGATTTCC | | |
| | | | GCTGATCTCGATACGTGACCAGGTGCAGGCGGACGTCG | | |
| | | | CGGCGCTGGCCGCGCTGGGCATCACCGAGGTCGCCGCC | | |
| | | | GTCGTCGGCGGCTCCATGGGCGGCGCCCGGGCCCTGGA | | |
| | | | ATGGGTGGTCGGCTACCCGGATCGGGTCCGAGCCGGAT | | |
| | | | TGCTGCTGGCGGTCGGTGCGCGTGCCACCGCAGACCAG | | |
| | | | ATCGGCACGCAGACAACGCAAATCGCGGCCATCAAAGC | | |
| | | | CGACCCGGACTGGCAGAGCGGCGACTACCACGAGACGG | | |
| | | | GGAGGGCACCAGACGCCGGGCTGCGACTCGCCCGCCGC | | |
| | | | TTCGCGCACCTCACCTACCGCGGCGAGATCGAGCTCGA | | |
| | | | CACCCGGTTCGCCAACCACAACCAGGGCAACGAGGATC | | |
| | | | CGACGGCCGGCGGGCGCTACGCGGTGCAAAGTTATCTG | | |
| | | | GAACACCAAGGAGACAAACTGTTATCCCGGTTCGACGC | | |
| | | | CGGCAGCTACGTGATTCTCACCGAGGCGCTCAACAGCC | | |
| | | | ACGACGTCGGCCGCGGCCGCGGCGGGGTCTCCGCGGCT | | |
| | | | CTGCGCGCCTGCCCGGTGCCGGTGGTGGTGGGCGGCAT | | |
| | | | CACCTCCGACCGGCTCTACCCGCTGCGCCTGCAGCAGG | | |
| | | | AGCTGGCCGACCTGCTGCCGGGCTGCGCCGGGCTGCGA | | |
| | | | GTCGTCGAGTCGGTCTACGGACACGACGGCTTCCTGGT | | |
| | | | GGAAACCGAGGCCGTGGGCGAATTGATCCGCCAGACAC | | |
| | | | TGGGATTGGCTGATCGTGAAGGCGCGTGTCGGCGG | | |
| metA | Mycobacterium | Z98271.1 | ATGACAATCTCCAAGGTCCCTACCCAGAAGCTGCCGGC | 51 | |
| | leprae (can be | | CGAAGGCGAGGTCGGCTTGGTCGACATCGGCTCACTTA | | |
| | used to clone | | CCACCGAAAGCGGTGCCGTCATCGACGATGTCTGCATC | | |
| | M. smegmatis | | GCCGTTCAGCGCTGGGGGGAATTGTCGCCCACGCGAGA | | |
| | gene) | | CAACGTAGTGATGGTACTGCATGCACTCACCGGTGACT | | |
| | | | CGCACATCACCGGGCCCGCCGGACCGGGACATCCCACA | | |
| | | | CCCGGCTGGTGGGACTGGATAGCTGGACCGGGTGCACC | | |
| | | | AATCGACACCAACCGCTGGTGCGCGATAGCCACCAACG | | |
| | | | TGCTGGGCGGTTGCCGTGGCTCCACCGGCCCTAGTTCG | | |
| | | | CTTGCCCGCGACGGAAAGCCTTGGGGTTCAAGATTTCC | | |
| | | | GCTGATATCTATACGCGACCAGGTAGAGGCAGATATCG | | |
| | | | CTGCACTGGCCGCCATGGGAATTACAAAGGTTGCCGCC | | |
| | | | GTCGTTGGAGGATCTATGGGCGGGGCGCGTGCACTGGA | | |
| | | | ATGGATCATCGGCCACCCGGACCAAGTCCGGGCCGGGC | | |
| | | | TGTTGCTGGCGGTCGGTGTGCGCGCCACCGCCGACCAG | | |
| | | | ATCGGCACCCAAACCACCCAAATCGCAGCCATCAAGAC | | |
| | | | AGACCCGAACTGGCAAGGCGGTGACTACTACGAGACAG | | |
| | | | GGAGGGCACCAGAGAACGGCTTGACAATTGCCCGCCGC | | |
| | | | TTCGCCCACCTGACCTACCGCAGCGAGGTCGAGCTCGA | | |
| | | | CACCCGGTTTGCCAACAACAACCAAGGCAATGAGGACC | | |
| | | | CGGCGACGGGCGGGCGTTACGCAGTGCAGAGTTACCTA | | |
| | | | GAGCACCAGGGTGACAAGCTATTGGCCCGCTTTGACGC | | |
| | | | AGGCAGCTACGTGGTCTTGACCGAAACGCTGAACAGCC | | |
| | | | ACGACGTTGGCCGGGGCCGCGGAGGGATCGGTACAGCG | | |
| | | | CTGCGCGGGTGCCCGGTACCGGTGGTGGTGGGTGGCAT | | |
| | | | TACCTCGGATCGGCTCTACCCACTGCGCTTGCAGCAGG | | |
| | | | AGCTGGCCGAGATGCTGCCGGGCTGCACCGGGCTGCAG | | |
| | | | GTTGTAGACTCCACCTACGGGCACGACGGCTTCCTGGT | | |
| | | | GGAATCCGAGGCCGTCGGCAAATTGATCCGTCAAACCC | | |
| | | | TCGAATTGGCCGACGTGGGTTCCAAGGAAGACGCGTGT | | |
| | | | TCGCAATGA | | |
| metA | Thermobifida | NZ_AAAQ010 | GTGAGTCACGACACCACCCCTCCCCTTCCCGCGACCGG | 52 | |
| | fusca | 00035.1 | CGCGTGGCGGGAAGGGGACCCTCCGGGCGACCGGCGCT | | |
| | | | GGGTCGAACTGTCCGAACCTCTGCCGCTGGAGACCGGG | | |
| | | | GGTGAACTTCCCGGGGTCCGCCTGGCCTACGAGACGTG | | |
| | | | GGGCAGTCTCAACGAGGACCGCTCCAACGCGGTCCTCG | | |
| | | | TGCTGCACGCCCTCACCGGCGACAGCCACGTCGTAGGC | | |
| | | | CCGGAAGGCCCCGGGCACCCCAGCCCAGGCTGGTGGGA | | |
| | | | AGGCATCATCGGCCCCGGGCTGGCACTCGACACCGACC | | |
| | | | GGTACTTCGTGGTCGCCCCCAACGTGCTGGGCGGCTGC | | |
| | | | CAAGGCAGCACCGGGCCGTCGTCGACCGCGCCCGACGG | | |
| | | | CAGGCCGTGGGGGTCCCGGTTCCCGAGGATCACCATCC | | |
| | | | GCGACACGGTGCGCGCCGAGTTCGCCCTGCTGCGCGAA | | |
| | | | TTCGGCATCCACTCGTGGGCCGCGGTCCTCGGCGGGTC | | |
| | | | CATGGGCGGGATGCGTGCCCTCGAATGGGCGGCCACCT | | |
| | | | ACCCGGAGCGGGTGCGTCGCCTCCTGCTGCTGGCCAGC | | |
| | | | CCTGCGGCCAGCTCCGCACAGCAGATCGCCTGGGCCGC | | |
| | | | CCCCCAGTTGCACGCCATCCGGTCTGATCCGTACTGGC | | |
| | | | ACGGTGGCGACTACTACGACCGTCCCGGTCCGGGACCG | | |
| | | | GTCACCGGCATGGGGATCGCCCGCCGTATCGCGCACAT | | |
| | | | CACCTACCGGGGTGCCACCGAGTTCGACGAACGGTTCG | | |
| | | | GCCGCAACCCCCAAGACGGGGAAGACCCGATGGCCGGG | | |
| | | | GGCCGGTTCGCTGTCGAGTCGTACCTGGACCACCACGC | | |
| | | | GGTCAAACTCGCCCGCCGGTTCGACGCGGGCAGCTACG | | |
| | | | TCGTGCTCACCCAAGCCATGAACACCCACGACGTGGGT | | |
| | | | CGGGGCCGCGGCGGGGTGGCGCAGGCGCTGCGCCGGGT | | |
| | | | CACCGCCCGCACCATGGTGGCCGGGGTGAGCAGCGACT | | |
| | | | TCCTGTACCCCCTCGCCCAGCAGCAGGAGCTCGCCGAC | | |
| | | | GGTATTCCCGGGGCCGACGAAGTCCGCGTCATCGAATC | | |
| | | | AGCCTCGGGCCACGACGGGTTCCTCACCGAGATC~CC | | |
| | | | AAGTGTCGGTCCTCATCAAAGAACTGCTGGCGCAG | | |
| metA | Coryne- | AF052652 | ATGCCCACCCTCGCGCCTTCAGGTCAACTTGAAATCCA | 250 | |
| | bacterium | | AGCGATCGGTGATGTCTCCACCGAAGCCGGAGCAATCA | | |
| | glutamicum | | TTACAAACGCTGAAATCGCCTATCACCGCTGGGGTGAA | | |
| | | | TACCGCGTAGATAAAGAAGGACGCAGCAATGTCGTTCT | | |
| | | | CATCGAACACGCCCTCACTGGAGATTCCAACGCAGCCG | | |
| | | | ATTGGTGGGCTGACTTGCTCGGTCCCGGCAAAGCCATC | | |
| | | | AACACTGATATTTACTGCGTGATCTGTACCAACGTCAT | | |
| | | | CGGTGGTTGCAACGGTTCCACCGGACCTGGCTCCATGC | | |
| | | | ATCCAGATGGAAATTTCTGGGGTAATCGCTTCCCCGCC | | |
| | | | ACGTCCATTCGTGATCAGGTAAACGCCGAAAAACAATT | | |
| | | | CCTCGACGCACTCGGCATCACCACGGTCGCCGCAGTAG | | |
| | | | TACTACTTGGTGGTTCCATGGGTGGTGCCCGCACCCTA | | |
| | | | GAGTGGGCCGCAATGTACCCAGAAACTGTTGGCGCAGC | | |
| | | | TGCTGTTCTTGCAGTTTCTGCACGCGCCAGCGCCTGGC | | |
| | | | AAATCGGCATTCAATCCGCCCAAATTAAGGCGATTGAA | | |
| | | | AACGACCACCACTGGCACGAAGGCAACTACTACGAATC | | |
| | | | CGGCTGCAACCCAGCCACCGGACTCGGCGCCGCCCGAC | | |
| | | | GCATCGCCCACCTCACCTACCGTGGCGAACTAGAAATC | | |
| | | | GACGAACGCTTCGGCACCAAAGCCCAAAAGAACGAAAA | | |
| | | | CCCACTCGGTCCCTACCGCAAGCCCGACCAGCGCTTCG | | |
| | | | CCGTGGAATCCTACTTGGACTACCAAGCAGACAAGCTA | | |
| | | | GTACAGCGTTTCGACGCCGGCTCCTACGTCTTGCTCAC | | |
| | | | CGACGCCCTCAACCGCCACGACATTGGTCGCGACCGCG | | |
| | | | GAGGCCTCAACAAGGCACTCGAATCCATCAAAGTTCCA | | |
| | | | GTCCTTGTCGCAGGCGTAGATACCGATATTTTGTACCC | | |
| | | | CTACCACCAGCAAGAACACCTCTCCAGAAACCTGGGAA | | |
| | | | ATCTACTGGCAATGGCAAAAATCGTATCCCCTGTCGGC | | |
| | | | CACGATGCTTTCCTCACCGAAAGCCGCCAAATGGATCG | | |
| | | | CATCGTGAGGAACTTCTTCAGCCTCATCTCCCCAGACG | | |
| | | | AAGACAACCCTTCGACCTACATCGAGTTCTACATCTAA | | |
| metA | Escherichia | NC_000913 | ATGCCGATTCGTGTGCCGGACGAGCTACCCGCCGTCAA | 251 | |
| | coli | | TTTCTTGCGTGAAGAAAACGTCTTTGTGATGACAACTT | | |
| | | | CTCGTGCGTCTGGTCAGGAAATTCGTCCACTTAAGGTT | | |
| | | | CTGATCCTTAACCTGATGCCGAAGAAGATTGAAACTGA | | |
| | | | AAATCAGTTTCTGCGCCTGCTTTCAAACTCACCTTTGC | | |
| | | | AGGTCGATATTCAGCTGTTGCGCATCGATTCCCGTGAA | | |
| | | | TCGCGCAACACGCCCGCAGAGCATCTGAACAACTTCTA | | |
| | | | CTGTAACTTTGAAGATATTCAGGATCAGAACTTTGACG | | |
| | | | GTTTGATTGTAACTGGTGCGCCGCTGGGCCTGGTGGAG | | |
| | | | TTTAATGATGTCGCTTACTGGCCGCAGATCAAACAGGT | | |
| | | | GCTGGAGTGGTCGAAAGATCACGTCACCTCGACGCTGT | | |
| | | | TTGTCTGCTGGGCGGTACAGGCCGCGCTCAATATCCTC | | |
| | | | TACGGCATTCCTAAGCAAACTCGCACCGAAAAACTCTC | | |
| | | | TGGCGTTTACGAGCATCATATTCTCCATCCTCATGCGC | | |
| | | | TTCTGACGCGTGGCTTTGATGATTCATTCCTGGCACCG | | |
| | | | CATTCGCGCTATGCTGACTTTCCGGCAGCGTTGATTCG | | |
| | | | TGATTACACCGATCTGGAAATTCTGGCAGAGACGGAAG | | |
| | | | AAGGGGATGCATATCTGTTTGCCAGTAAAGATAAGCGC | | |
| | | | ATTGCCTTTGTGACGGGCCATCCCGAATATGATGCGCA | | |
| | | | AACGCTGGCGCAGGAATTTTTCCGCGATGTGGAAGCCG | | |
| | | | GACTAGACCCGGATGTACCGTATAACTATTTCCCGCAC | | |
| | | | AATGATCCGCAAAATACACCGCGAGCGAGCTGGCGTAG | | |
| | | | TCACGGTAATTTACTGTTTACCAACTGGCTCAACTATT | | |
| | | | ACGTCTACCAGATCACGCCATACGATCTACGGCACATG | | |
| | | | AATCCAACGCTGGAT | | |
| metA K233A | C. glutamicum | n/a | atgcccaccctcgcgccttcaggtcaacttgaaatccaagcg | 294 | |
| | | | atcggtgatgtctccaccgaagccggagcaatcattacaaac | | |
| | | | gctgaaatcgcctatcaccgctggggtgaataccgcgtagat | | |
| | | | aaagaaggacgcagcaatgtcgttctcatcgaacacgccctc | | |
| | | | actggagattccaacgcagccgattggtgggctgacttgctc | | |
| | | | ggtcccggcaaagccatcaacactgatatttactgcgtgatc | | |
| | | | tgtaccaacgtcatcggtggttgcaacggttccaccggacct | | |
| | | | ggctccatgcatccagatggaaatttctggggtaatcgcttc | | |
| | | | cccgccacgtccattcgtgatcaggtaaacgccgaaaaacaa | | |
| | | | ttcctcgacgcactcggcatcaccacggtcgccgcagtactt | | |
| | | | ggtggttccatgggtggtgcccgcaccctagagtgggccgca | | |
| | | | atgtacccagaaactgttggcgcagctgctgttcttgcagtt | | |
| | | | tctgcacgcgccagcgcctggcaaatcggcattcaatccgcc | | |
| | | | caaattaaggcgattgaaaacgaccaccactggcacgaaggc | | |
| | | | aactactacgaatccggctgcaacccagccaccggactcggc | | |
| | | | gccgcccgacgcatcgcccacctcacctaccgtggcgaacta | | |
| | | | gaaatcgacgaacgcttcggcaccgcagcccaaaagaacgaa | | |
| | | | aacccactcggtccctaccgcaagcccgaccagcgcttcgcc | | |
| | | | gtggaatcctacttggactaccaagcagacaagctagtacag | | |
| | | | cgtttcgacgccggctcctacgtcttgctcaccgacgccctc | | |
| | | | aaccgccacgacattggtcgcgaccgcggaggcctcaacaag | | |
| | | | gcactcgaatccatcaaagttccagtccttgtcgcaggcgta | | |
| | | | gataccgatattttgtacccctaccaccagcaagaacacctc | | |
| | | | tccagaaacctgggaaatctactggcaatggcaaaaatcgta | | |
| | | | tcccctgtcggccacgatgctttcctcaccgaaagccgccaa | | |
| | | | atggatcgcatcgtgaggaacttcttcagcctcatctcccca | | |
| | | | gacgaagacaacccttcgacctacatcgagttctacatctaa | | |
| metY | Thermobifida | NZ_AAAQ010 | GTGGCACTGCGTCCTGACAGGAGCATCATGACCGCTGA | 53 | |
| | fusca | 00035.1 | AGACACCACGCCTGAATCCACCGCGGCCGACAAGTGGT | | |
| | | | CGTTCGAAACCAAGCAGATCCACGCCGGAGCGGCCCCC | | |
| | | | GATCCGGCCACCAACGCACGGGCCACCCCCATCTACCA | | |
| | | | GACCACGTCGTACGTCTTCCGGGACACGCAGCACGGGG | | |
| | | | CCGACCTGTTCTCGCTCGCAGAGCCGGGCAACATCTAC | | |
| | | | ACGCGGATCATGAACCCCACCCAGGACGTGCTGGAAAA | | |
| | | | GCGGGTCGCGGCTCTGGAAGGCGGGGTCGCCGCGGTCG | | |
| | | | CGTTCGCGTCCGGGTCAGCTGCCATCACCGCTGCCGTC | | |
| | | | CTCAACCTGGCGGGTGCGGGTGACCACATCGTGTCCAG | | |
| | | | CCCGTCCCTGTACGGCGGCACCTACAACCTGTTCCGCT | | |
| | | | ACACCCTGCCCAAGCTCGGCATCGAGGTCACCTTCATC | | |
| | | | AAAGACCAGGACGACCTCGACGAGTGGCGTGCCGCGGC | | |
| | | | CCGCGACAACACCAAGCTGTTCTTCGCGGAAACCCTGC | | |
| | | | CCAACCCGGCGAACAACGTGCTCGACGTGCGCGCGGTG | | |
| | | | GCGGACGTCGCCCACGAGGTCGGTGTGCCGCTCATGGT | | |
| | | | CGACAACACCGTGCCCACCCCCTACCTGCAGCGGCCCA | | |
| | | | TCGACCACGGCGCGGACATCGTGGTGCACTCGGCCACC | | |
| | | | AAGTTCCTCGGCGGCCACGGCACCACGATCGCGGGCAT | | |
| | | | CGTGGTGGACGCCGGCACCTTCGACTTCGGCGCCCACG | | |
| | | | GCGACCGGTTCCCCGGCTTCGTCGAACCCGACCCCAGC | | |
| | | | TACCATGGCCTGAAGTACTGGGAGGCGCTGGGACCGGG | | |
| | | | TGCCTACGCTGCCAAGCTGCGGGTGCAACTGCTCCGCG | | |
| | | | ACACGGGCGCGGCCATCTCGCCGTTCAACAGCTTCCTG | | |
| | | | ATCCTCCAGGGGATCGAAACGCTGTCGCTGCGCATGGA | | |
| | | | ACGGCACGTCGCCAACGCCCAGGCGCTCGCCGAGTGGC | | |
| | | | TGGAATCCCGCGACGAGGTGGCGAAGGTCTACTACCCG | | |
| | | | GGCCTGCCTTCCAGCCCCTACTACGAGGCTGCAAAGAA | | |
| | | | GTACCTGCCCAAGGGGGCGGGTGCGATCGTCTCCTTTG | | |
| | | | AGCTGCACGGCGGTATCGAGGCCGGACGCGCCTTCGTG | | |
| | | | GACGGCACCGAACTGTTCAGCCAGCTCGTCAACATCGG | | |
| | | | TGACGTGCGCAGCCTCATCGTCCACCCGGCCAGCACCA | | |
| | | | CGCACAGCCAGCTCACCCCCGAAGAGCAGCTCGCCAGc | | |
| | | | GGGGTCACTCCCGGCCTCGTGCGGCTGTCCGTGGGCTT | | |
| | | | GGAACACGTTGACGACCTTCGCGCAGACTTGGAGGCCG | | |
| | | | GGCTGCGCGCAGCCAAGGCATACCAGTGA | | |
| metY | Mycobacterium | AL021841.1 | ATGAGCGCCGACAGCAATAGCACCGACGCCGATCCGAC | 54 | |
| | tuberculosis | | CGCGCATTGGTCGTTCGAAACCAAACAGATACACGCTG | | |
| | (can be used to | | GTCAGCACCCTGATCCGACCACCAACGCCCGGGCTCTG | | |
| | clone M. | | CCGATCTATGCGACCACGTCGTACACCTTCGACGACAC | | |
| | smegmatis | | CGCGCACGCCGCCGCCCTGTTCGGACTGGAAATTCCGG | | |
| | gene) | | GCAATATCTACACCCGGATCGGCAACCCCACCACCGAC | | |
| | | | GTCGTCGAGCAGCGCATCGCCGCGCTCGAGGGCGGTGT | | |
| | | | GGCCGCGCTGTTCCTGTCGTCGGGGCAGGCCGCGGAGA | | |
| | | | CGTTCGCCATCTTGAACCTGGCCGGCGCGGGCGATCAC | | |
| | | | ATCGTGTCCAGCCCGCGCCTGTACGGCGGCACCTACAA | | |
| | | | CCTGTTCCACTATTCGCTGGCCAAGCTCGGCATCGAGG | | |
| | | | TCAGCTTCGTCGACGATCCGGACGATCTGGACACCTGG | | |
| | | | CAGGCGGCGGTACGGCCCAACACCAAGGCGTTCTTCGC | | |
| | | | CGAGACCATCTCCAACCCGCAGATCGACCTGCTGGACA | | |
| | | | CCCCGGCGGTTTCCGAGGTCGCCCATCGCAACGGGGTG | | |
| | | | CCGTTGATCGTCGACAACACCATCGCCACGCCATACCT | | |
| | | | GATCCAACCGTTGGCCCAGGGCGCCGACATCGTCGTGC | | |
| | | | ATTCGGCCACCAAGTACCTGGGCGGGCACGGTGCCGCC | | |
| | | | ATCGCGGGTGTGATCGTCGACGGCGGCAACTTCGATTG | | |
| | | | GACCCAGGGCCGCTTCCCCGGCTTCACCACCCCCGACC | | |
| | | | CCAGCTACCACGGCGTGGTGTTCGCCGAGCTGGGTCCA | | |
| | | | CCGGCGTTTGCGCTCAAAGCTCGAGTGCAGCTGCTCCG | | |
| | | | TGACTACGGCTCGGCGGCTTCGCCGTTCAACGCGTTCT | | |
| | | | TGGTGGCGCAGGGTCTGGAAACGCTGAGCCTGCGGATC | | |
| | | | GAGCGGCACGTCGCCAACGCGCAGCGCGTCGCCGAGTT | | |
| | | | CCTGGCCGCCCGCGACGACGTGCTTTCGGTCAACTATG | | |
| | | | CGGGGCTGCCCTCCTCGCCCTGGCATGAGCGGGCCAAG | | |
| | | | AGGCTGGCGCCCAAGGGAACCGGGGCCGTGCTGTCCTT | | |
| | | | CGAGTTGGCCGGCGGCATCGAGGCCGGCAAGGCATTCG | | |
| | | | TGAACGCGTTGAAGCTGCACAGCCACGTCGCCAACATC | | |
| | | | GGTGACGTGCGCTCGCTGGTGATCCACCCGGCATCGAC | | |
| | | | CACTCATGCCCAGCTGAGCCCGGCCGAGCAGCTGGCGA | | |
| | | | CCGGGGTCAGCCCGGGCCTGGTGCGTTTGGCTGTGGGC | | |
| | | | ATCGAAGGTATCGACGATATCCTGGCCGACCTGGAGCT | | |
| | | | TGGCTTTGCCGCGGCCCGCAGATTCAGCGCCGACCCGC | | |
| | | | AGTCCGTGGCGGCGTTCTGA | | |
| metY | Coryne- | AF220150 | ATGCCAAAGTACGACAATTCCAATGCTGACCAGTGGGG | 252 | |
| | bacterium | | CTTTGAAACCCGCTCCATTCACGCAGGCCAGTCAGTAG | | |
| | glutamicum | | ACGCACAGACCAGCGCACGAAACCTTCCGATCTACCAA | | |
| | | | TCCACCGCTTTCGTGTTCGACTCCGCTGAGCACGCCAA | | |
| | | | GCAGCGTTTCGCACTTGAGGATCTAGGCCCTGTTTACT | | |
| | | | CCCGCCTCACCAACCCAACCGTTGAGGCTTTGGAAAAC | | |
| | | | CGCATCGCTTCCCTCGAAGGTGGCGTCCACGCTGTAGC | | |
| | | | GTTCTCCTCCGGACAGGCCGCAACCACCAACGCCATTT | | |
| | | | TGAACCTGGCAGGAGCGGGCGACCACATCGTCACCTCC | | |
| | | | CCACGCCTCTACGGTGGCACCGAGACTCTATTCCTTAT | | |
| | | | CACTCTTAACCGCCTGGGTATCGATGTTTCCTTCGTGG | | |
| | | | AAAACCCCGACGACCCTGAGTCCTGGCAGGCAGCCGTT | | |
| | | | CAGCCAAACACCAAAGCATTCTTCGGCGAGACTTTCGC | | |
| | | | CAACCCACAGGCAGACGTCCTGGATATTCCTGCGGTGG | | |
| | | | CTGAAGTTGCGCACCGCAACAGCGTTCCACTGATCATC | | |
| | | | GACAACACCATCGCTACCGCAGCGCTCGTGCGCCCGCT | | |
| | | | CGAGCTCGGCGCAGACGTTGTCGTCGCTTCCCTCACCA | | |
| | | | AGTTCTACACCGGCAACGGCTCCGGACTGGGCGGCGTG | | |
| | | | CTTATCGACGGCGGAAAGTTCGATTGGACTGTCGAAAA | | |
| | | | GGATGGAAAGCCAGTATTCCCCTACTTCGTCACTCCAG | | |
| | | | ATGCTGCTTACCACGGATTGAAGTACGCAGACCTTGGT | | |
| | | | GCACCAGCCTTCGGCCTCAAGGTTCGCGTTGGCCTTCT | | |
| | | | ACGCGACACCGGCTCCACCCTCTCCGCATTCAACGCAT | | |
| | | | GGGCTGCAGTCCAGGGCATCGACACCCTTTCCCTGCGC | | |
| | | | CTGGAGCGCCACAACGAAAACGCCATCAAGGTTGCAGA | | |
| | | | ATTCCTCAACAACCACGAGAAGGTGGAAAAGGTTAACT | | |
| | | | TCGCAGGCCTGAAGGATTCCCCTTGGTACGCAACCAAG | | |
| | | | GAAAAGCTTGGCCTGAAGTACACCGGCTCCGTTCTCAC | | |
| | | | CTTCGAGATCAAGGGCGGCAAGGATGAGGCTTGGGCAT | | |
| | | | TTATCGACGCCCTGAAGCTACACTCCAACCTTGCAAAC | | |
| | | | ATCGGCGATGTTCGCTCCCTCGTTGTTCACCCAGCAAC | | |
| | | | CACCACCCATTCACAGTCCGACGAAGCTGGCCTGGCAC | | |
| | | | GCGCGGGCGTTACCCAGTCCACCGTCCGCCTGTCCGTT | | |
| | | | GGCATCGAGACCATTGATGATATCATCGCTGACCTCGA | | |
| | | | AGGCGGCTTTGCTGCAATCTAG | | |
| metY D231A | C. glutamicum | N/a | ATGCCAAAGTACGACAATTCCAATGCTGACCAGTGGGGCTTT | 295 | |
| | | | GAAACCCGCTCCATTCACGCAGGCCAGTCAGTAGACGCACAG | | |
| | | | ACCAGCGCACGAAACCTTCCGATCTACCAATCCACCGCTTTC | | |
| | | | GTGTTCGACTCCGCTGAGCACGCCAAGCAGCGTTTCGCACTT | | |
| | | | GAGGATCTAGGCCCTGTTTACTCCCGCCTCACCAACCCAACC | | |
| | | | GTTGAGGCTTTGGAAAACCGCATCGCTTCCCTCGAAGGTGGC | | |
| | | | GTCCACGCTGTAGCGTTCTCCTCCGGACAGGCCGCAACCACC | | |
| | | | AACGCCATTTTGAACCTGGCAGGAGCGGGCGACCACATCGTC | | |
| | | | ACCTCCCCACGCCTCTACGGTGGCACCGAGACTCTATTCCTT | | |
| | | | ATCACTCTTAACCGCCTGGGTATCGATGTTTCCTTCGTGGAA | | |
| | | | AACCCCGACGACCCTGAGTCCTGGCAGGCAGCCGTTCAGCCA | | |
| | | | AACACCAAAGCATTCTTCGGCGAGACTTTCGCCAACCCACAG | | |
| | | | GCAGACGTCCTGGATATTCCTGCGGTGGCTGAAGTTGCGCAC | | |
| | | | CGCAACAGCGTTCCACTGATCATCGACAACACCATCGCTACC | | |
| | | | GCAGCGCTCGTGCGCCCGCTCGAGCTCGGCGCAGACGTTGTC | | |
| | | | GTCGCTTCCCTCACCAAGTTCTACACCGGCAACGGCTCCGGA | | |
| | | | CTGGGCGGCGTGCTTATCGCCGGCGGAAAGTTCGATTGGACT | | |
| | | | GTCGAAAAGGATGGAAAGCCAGTATTCCCCTACTTCGTCACT | | |
| | | | CCAGATGCTGCTTACCACGGATTGAAGTACGCAGACCTTGGT | | |
| | | | GCACCAGCCTTCGGCCTCAAGGTTCGCGTTGGCCTTCTACGC | | |
| | | | GACACCGGCTCCACCCTCTCCGCATTCAACGCATGGGCTGCA | | |
| | | | GTCCAGGGCATCGACACCCTTTCCCTGCGCCTGGAGCGCCAC | | |
| | | | AACGAAAACGCCATCAAGGTTGCAGAATTCCTCAACAACCAC | | |
| | | | GAGAAGGTGGAAAAGGTTAACTTCGCAGGCCTGAAGGATTCC | | |
| | | | CCTTGGTACGCAACCAAGGAAAAGCTTGGCCTGAAGTACACC | | |
| | | | GGCTCCGTTCTCACCTTCGAGATCAAGGGCGGCAAGGATGAG | | |
| | | | GCTTGGGCATTTATCGACGCCCTGAAGCTACACTCCAACCTT | | |
| | | | GCAAACATCGGCGATGTTCGCTCCCTCGTTGTTCACCCAGCA | | |
| | | | ACCACCACCCATTCACAGTCCGACGAAGCTGGCCTGGCACGC | | |
| | | | GCGGGCGTTACCCAGTCCACCGTCCGCCTGTCCGTTGGCATC | | |
| | | | GAGACCATTGATGATATCATCGCTGACCTCGAAGGCGGCTTT | | |
| | | | GCTGCAATCTAG | | |
| metY G232A | C. glutamicum | N/a | ATGCCAAAGTACGACAATTCCAATGCTGACCAGTGGGGCTTT | 296 | |
| | | | GAAACCCGCTCCATTCACGCAGGCCAGTCAGTAGACGCACAG | | |
| | | | ACCAGCGCACGAAACCTTCCGATCTACCAATCCACCGCTTTC | | |
| | | | GTGTTCGACTCCGCTGAGCACGCCAAGCAGCGTTTCGCACTT | | |
| | | | GAGGATCTAGGCCCTGTTTACTCCCGCCTCACCAACCCAACC | | |
| | | | GTTGAGGCTTTGGAAAACCGCATCGCTTCCCTCGAAGGTGGC | | |
| | | | GTCCACGCTGTAGCGTTCTCCTCCGGACAGGCCGCAACCACC | | |
| | | | AACGCCATTTTGAACCTGGCAGGAGCGGGCGACCACATCGTC | | |
| | | | ACCTCCCCACGCCTCTACGGTGGCACCGAGACTCTATTCCTT | | |
| | | | ATCACTCTTAACCGCCTGGGTATCGATGTTTCCTTCGTGGAA | | |
| | | | AACCCCGACGACCCTGAGTCCTGGCAGGCAGCCGTTCAGCCA | | |
| | | | AACACCAAAGCATTCTTCGGCGAGACTTTCGCCAACCCACAG | | |
| | | | GCAGACGTCCTGGATATTCCTGCGGTGGCTGAAGTTGCGCAC | | |
| | | | CGCAACAGCGTTCCACTGATCATCGACAACACCATCGCTACC | | |
| | | | GCAGCGCTCGTGCGCCCGCTCGAGCTCGGCGCAGACGTTGTC | | |
| | | | GTCGCTTCCCTCACCAAGTTCTACACCGGCAACGGCTCCGGA | | |
| | | | CTGGGCGGCGTGCTTATCGACGCCGGAAAGTTCGATTGGACT | | |
| | | | GTCGAAAAGGATGGAAAGCCAGTATTCCCCTACTTCGTCACT | | |
| | | | CCAGATGCTGCTTACCACGGATTGAAGTACGCAGACCTTGGT | | |
| | | | GCACCAGCCTTCGGCCTCAAGGTTCGCGTTGGCCTTCTACGC | | |
| | | | GACACCGGCTCCACCCTCTCCGCATTCAACGCATGGGCTGCA | | |
| | | | GTCCAGGGCATCGACACCCTTTCCCTGCGCCTGGAGCGCCAC | | |
| | | | AACGAAAACGCCATCAAGGTTGCAGAATTCCTCAACAACCAC | | |
| | | | GAGAAGGTGGAAAAGGTTAACTTCGCAGGCCTGAAGGATTCC | | |
| | | | CCTTGGTACGCAACCAAGGAAAAGCTTGGCCTGAAGTACACC | | |
| | | | GGCTCCGTTCTCACCTTCGAGATCAAGGGCGGCAAGGATGAG | | |
| | | | GCTTGGGCATTTATCGACGCCCTGAAGCTACACTCCAACCTT | | |
| | | | GCAAACATCGGCGATGTTCGCTCCCTCGTTGTTCACCCAGCA | | |
| | | | ACCACCACCCATTCACAGTCCGACGAAGCTGGCCTGGCACGC | | |
| | | | GCGGGCGTTACCCAGTCCACCGTCCGCCTGTCCGTTGGCATC | | |
| | | | GAGACCATTGATGATATCATCGCTGACCTCGAAGGCGGCTTT | | |
| | | | GCTGCAATCTAG | | |
| metK | Mycobacterium | Z80108.1 | GTGAGCGAAAAGGGTCGGCTGTTTACCAGTGAGTCGGT | 55 | |
| | tuberculosis | | GACAGAGGGACATCCCGACAAGATCTGTGACGCCATCA | | |
| | (can be used to | | GCGACTCGGTTCTGGACGCGCTTCTAGCGGCGGACCCG | | |
| | clone M. | | CGCTCACGTGTCGCGGTCGAGACGCTGGTGACCACCGG | | |
| | smegmatis | | GCAGGTGCACGTGGTGGGTGAGGTGACCACCTCGGCTA | | |
| | gene) | | AGGAGGCGTTTGCCGACATCACCAACACGGTCCGCGCA | | |
| | | | CGGATCCTCGAGATCGGCTACGACTCGTCGGACAAGGG | | |
| | | | TTTCGACGGGGCGACCTGCGGGGTGAACATCGGCATCG | | |
| | | | GCGCACAGTCACCCGACATCGCCCAGGGGGTCGACACC | | |
| | | | GCCCACGAGGCCCGGGTCGAGGGCGCGGCCGATCCGCT | | |
| | | | GGACTCCCAGGGCGCCGGTGACCAGGGCCTGATGTTCG | | |
| | | | GCTACGCGATCAATGCCACCCCGGAACTGATGCCACTG | | |
| | | | CCCATCGCGCTGGCCCACCGACTGTCGCGGCGGCTGAC | | |
| | | | CGAGGTCCGCAAGAACGGGGTGCTGCCCTACCTGCGTC | | |
| | | | CGGATGGCAAGACGCAGGTCACTATCGCCTACGAGGAC | | |
| | | | AACGTTCCGGTGCGGCTGGATACCGTGGTCATCTCCAC | | |
| | | | CCAGCACGCGGCCGATATCGACCTGGAGAAGACGCTTG | | |
| | | | ATCCCGACATCCGGGAAAAGGTGCTCAACACCGTGCTC | | |
| | | | GACGACCTGGCCCACGAAACCCTGGACGCGTCGACGGT | | |
| | | | GCGGGTGCTGGTGAACCCGACCGGCAAGTTCGTGCTCG | | |
| | | | GCGGGCCGATGGGCGATGCCGGGCTCACCGGCCGCAAG | | |
| | | | ATCATCGTCGACACCTACGGCGGCTGGGCCCGCCACGG | | |
| | | | CGGCGGCGCCTTCTCCGGCAAGGATCCGTCCAAGGTGG | | |
| | | | ACCGGTCGGCGGCGTACGCGATGCGCTGGGTGGCCAAG | | |
| | | | AATGTCGTCGCCGCCGGGTTGGCTGAACGGGTCGAGGT | | |
| | | | GCAGGTGGCCTACGCCATCGGTAAAGCGGCACCCGTCG | | |
| | | | GCCTGTTCGTCGAGACGTTCGGTACCGAGACGGAAGAC | | |
| | | | CCGGTCAAGATCGAGAAGGCCATCGGCGAGGTATTCGA | | |
| | | | CCTGCGCCCCGGTGCCATCATCCGCGACCTGAACCTGT | | |
| | | | TGCGCCCGATCTATGCGCCGACCGCCGCCTACGGGCAC | | |
| | | | TTCGGCCGCACCGACGTCGAATTACCGTGGGAGCAGCT | | |
| | | | CGACAAGGTCGACGACCTCAAGCGCGCCATCTAG | | |
| metK | Mycobacterium | AL583918.1 | GTGAGTGAGAAGGGTCGGCTGTTCACTAGCGAGTCGGT | 56 | |
| | leprae (can be | | GACTGAGGGACATCCCGACAAGATCTGTGATGCGATCA | | |
| | used to clone | | GCGACTCGATCCTTGACGCACTTTTGGCGGAGGATCCT | | |
| | M. smegmatis | | TGCTCACGTGTCGCGGTCGAGACGTTGGTCACCACCGG | | |
| | gene) | | GCAGGTGCATGTGGTGGGTGAAGTGACGACGTTGGCCA | | |
| | | | AGACGGCGTTCGCTGATATCAGTAATACGGTCCGCGAA | | |
| | | | CGTATTCTCGATATCGGCTACGACTCGTCGGACAAGGG | | |
| | | | CTTCGATGGGGCGTCGTGCGGAGTTAACATTGGCATCG | | |
| | | | GCGCTCAGTCGTCTGACATTGCTCAAGGCGTCAATACC | | |
| | | | GCCCATGAAGTACGCGTCGAGGGCGCGGCGGATCCGCT | | |
| | | | GGACGCCCAGGGTGCTGGTGACCAAGGCCTGATGTTCG | | |
| | | | GTTACGCGATCAATGACACCCCGGAACTGATGCCGCTA | | |
| | | | CCGATTGCACTGGCCCACCGACTGGCGCGAAGGCTGAC | | |
| | | | CGAGGTACGCAAGAACGGCGTGCTGCCCTACCTGCGTT | | |
| | | | CCGACGGCAAGACCCAGGTCACTATCGCCTACGAGGAC | | |
| | | | AATGTCCCAGTGCGTTTGGACACTGTGGTCATCTCcAC | | |
| | | | TCAGCACGCCGCTGGTGTCGACCTGGATGCCACGCTGG | | |
| | | | CTCCTGATATCCGGGAGAAGGTGCTCAACACCGTTATT | | |
| | | | GACGATCTGTCTCATGACACCTTGGATGTATCGTCGGT | | |
| | | | GCGGGTGCTGGTAAACCCGACCGGCAAGTTCGTGCTAG | | |
| | | | GTGGGCCGATGGGCGATGCCGGGCTCACCGGTCGCAAG | | |
| | | | ATCATCGTCGACACCTACGGTGGCTGGGCGCGTCACGG | | |
| | | | CGGCGGCGCCTTCTCTGGCAAGGATCCGTCCAAGGTGG | | |
| | | | ACCGGTCGGCAGCCTACGCGATGCGCTGGGTGGCCAAG | | |
| | | | AACATCGTCGCTGCCGGGCTGGCGGAGCGAATCGAGGT | | |
| | | | GCAGGTGGCATACGCCATCGGCAAAGCCGCCCCGGTCG | | |
| | | | GTTTGTTCGTCGAGACCTTTGGCACTGAGGCGGTCGAT | | |
| | | | CCGGCCAAAATCGAGAAAGCCATCGGCGAGGTGTTCGA | | |
| | | | TCTGCGTCCCGGCGCGATCATCCGCGACCTGCATCTGC | | |
| | | | TGCGCCCAATTTACGCGCAAACCGCTGCCTATGGGCAC | | |
| | | | TTCGGTCGCACTGACGTCGAACTGCCATGGGAGCAGCT | | |
| | | | CAACAAAGTCGACGATCTCAAGCGCGCCATC | | |
| metK | Thermobifida | NZ_AAAQ010 | GTGTCCCGTCGACTTTTCACCTCCGAGTCGGTCACCGA | 57 | |
| | fusca | 00031.1 | AGGCCACCCCGACAAGATCGCTGACCAGATCAGTGACG | | |
| | | | CGATCCTCGACTCGATGCTCAGGGATGACCCCCACAGC | | |
| | | | CGGGTCGCGGTGGAGACCCTCATCACGACCGGCCTGGT | | |
| | | | CCACGTCGCCGGCGAAGTGACCACATCCACCTACGTCG | | |
| | | | ACATTCCCACCATCATCCGCGAGAAGATCCTGGAGATC | | |
| | | | GGCTACGACTCCTCGGCCAAGGGGTTCGACGGCGCCTC | | |
| | | | CTGCGGAGTGTCCGTGTCGATCGGCGGGCAGTCACCCG | | |
| | | | ACATCGCCCAGGGCGTCGACAACGCCTACGAGGCCCGG | | |
| | | | GAGGAAGAGATCTTCGACGACCTCGACCGGCAGGGCGC | | |
| | | | AGGCGACCAAGGCCTCATGTTCGGCTACGCCAACAACG | | |
| | | | AGACCCCGGAGCTGATGCCGCTGCCGATCACGCTGGCC | | |
| | | | CACGCCCTGTCGCAGCGACTCGCTGAAGTGCGCCGCGA | | |
| | | | CGGGACCATCCCCTACCTGCGGCCCGACGGCAAGACCC | | |
| | | | AGGTCACCGTGGAGTACGACGGGAACCGGCCCGTCCGG | | |
| | | | TTGGACACCGTGGTGGTCTCCAGCCAGCACGCGCCCGA | | |
| | | | CATCGACCTGCGGGAACTGCTCACCCCGGACATCAAGG | | |
| | | | AGCACGTGGTCGACCCGGTAGTGGCCCGCTACAACCTG | | |
| | | | GAGGCCGACAACTACCGACTGCTCGTCAACCCCACCGG | | |
| | | | ACGGTTCGAGATCGGCGGCCCGATGGGTGACGCCGGGC | | |
| | | | TGACCGGCCGCAAGATCATCGTCGACACCTACGGCGGC | | |
| | | | TACGCCCGCCACGGCGGTGGCGCGTTCTCCGGCAAGGA | | |
| | | | CCCGTCCAAGGTGGACCGCTCCGCCGCGTACGCCACCC | | |
| | | | GCTGGGTCGCGAAGAACATCGTCGCCGCCGGGCTCGCC | | |
| | | | GACCGAGTCGAAGTCCAGGTCGCCTACGCGATCGGCAA | | |
| | | | AGCCCACCCGGTCGGCGTGTTCCTGGAGACCTTCGGCA | | |
| | | | CCGAGAAGGTCGCCCCGGAGCAGTTGGAGAAGGCGGTG | | |
| | | | CTGGAGGTCTTCGACCTGCGTCCCGCCGCGATCATCCG | | |
| | | | CGACCTGGACCTGCTGCGCCCCATCTACTCCCAGACCT | | |
| | | | CGGTCTACGGCCACTTCGGCCGGGAGCTGCCCGACTTC | | |
| | | | ACCTGGGAGCGCACCGACCGCGTCGACGCTCTCAAGGC | | |
| | | | TGCCGTGGGCGCCTGA | | |
| metk | Streptomyces | AL939109.1 | GTGTCCCGTCGCCTGTTCACCTCGGAGTCCGTGACCGA | 58 | |
| | coelicolor | | AGGTCACCCCGACAAGATCGCTGACCAGATCAGCGACA | | |
| | | | CGATTCTCGACGCGCTTCTGCGCGAGGACCCGACCTCC | | |
| | | | CGGGTCGCCGTCGAAACCCTGATCACCACCGGTCTGGT | | |
| | | | GCACGTGGCCGGCGAGGTCACCACCAAGGCCTACGCGG | | |
| | | | ACATCGCCAACCTGGTCCGCGGCAAGATCCTGGAGATC | | |
| | | | GGCTACGACTCCTCCAAGAAGGGCTTCGACGGCGCCTC | | |
| | | | CTGCGGCGTCTCGGTCTCCATCGGCGCGCAGTCCCCGG | | |
| | | | ACATCGCGCAGGGCGTCGACACGGCGTACGAGAACCGG | | |
| | | | GTGGAGGGCGACGAGGACGAGCTGGACCGCCAGGGTGC | | |
| | | | CGGCGACCAGGGCCTGATGTTCGGCTACGCGTCCGACG | | |
| | | | AGACGCCGACGCTGATGCCGCTGCCGGTCTTCCTGGCG | | |
| | | | CACCGCCTGTCCAAGCGCCTGTCCGAGGTCCGCAAGAA | | |
| | | | CGGCACCATCCCGTACCTGCGTCCGGACGGCAAGACCC | | |
| | | | AGGTCACCATCGAGTACGACGGCGACAAGGCCGTCCGT | | |
| | | | CTGGACACGGTCGTCGTCTCCTCCCAGCACGCGAGCGA | | |
| | | | CATCGACCTGGAGTCGCTGCTGGCGCCGGACATCAAGG | | |
| | | | AGTTCGTCGTCGAGCCGGAGCTGAAGGCGCTCCTCGAG | | |
| | | | GACGGCATCAAGATCGACACGGAGAACTACCGCCTCCT | | |
| | | | GGTCAACCCGACCGGCCGCTTCGAGATCGGCGGCCCGA | | |
| | | | TGGGCGACGCCGGTCTGACCGGCCGCAAGATCATCATC | | |
| | | | GACACCTACGGCGGCATGGCCCGGCACGGCGGCGGCGC | | |
| | | | CTTCTCCGGCAAGGACCCGTCGAAGGTCGACCGCTCCG | | |
| | | | CGGCGTACGCGATGCGCTGGGTCGCCAAGAACGTCGTG | | |
| | | | GCCGCGGGTCTCGCCGCGCGCTGCGAGGTCCAGGTCGC | | |
| | | | CTACGCCATCGGCAAGGCCGAGCCCGTGGGTCTGTTCG | | |
| | | | TGGAGACCTTCGGTACCGCCAAGGTCGACACCGAGAAG | | |
| | | | ATCGAGAAGGCGATCGACGAGGTCTTCGACCTGCGCCC | | |
| | | | GGCCGCCATCATCCGCGCTCTCGACCTGCTCCGCCCGA | | |
| | | | TCTACGCCCAGACCGCGGCGTACGGTCACTTCGGCCGT | | |
| | | | GAGCTGCCCGACTTCACGTGGGAGCGCACCGACCGCGT | | |
| | | | GGACGCGCTGCGCGAGGCCGCGGGCCTGTAA | | |
| metK | Coryne- | AP005279 | GTGGCTCAGCCAACCGCCGTCCGTTTGTTCACCAGTGA | 253 | |
| | bacterium | | ATCTGTAACTGAGGGACATCCAGACAAAATATGTGATG | | |
| | glutamicum | | CTATTTCCGATACCATTTTGGACGCGCTGCTCGAAAAA | | |
| | | | GATCCGCAGTCGCGCGTCGCAGTGGAAACTGTGGTCAC | | |
| | | | CACCGGAATCGTCCATGTTGTTGGCGAGGTCCGTACCA | | |
| | | | GCGCTTACGTAGAGATCCCTCAATTAGTCCGCAACAAG | | |
| | | | CTCATCGATATCGGATTCAACTCCTCTGAGGTTGGATT | | |
| | | | CGACGGACGCACCTGTGGCGTCTCAGTATCCATCGGTG | | |
| | | | AGCAGTCCCAGGAAATCGCTGACGGCGTGGATAACTCC | | |
| | | | GACGAAGCCCGCACCAACGGCGACGTTGAAGAAGACGA | | |
| | | | CCGCGCAGGTGCTGGCGACCAGGGCCTGATGTTCGGCT | | |
| | | | ACGCCACCAACGAAACCGAAGAGTACATGCCTCTTCCT | | |
| | | | ATCGCGTTGGCGCACCGACTGTCACGTCGTCTGACCCA | | |
| | | | GGTTCGTAAAGAGGGCATCGTTCCTCACCTGCGTCCAG | | |
| | | | ACGGAAAAACCCAGGTCACCTTCGCATACGATGCGCAA | | |
| | | | GACCGCCCTAGCCACCTGGATACCGTTGTCATCTCCAC | | |
| | | | CCAGCACGACCCAGAAGTTGACCGTGCATGGTTGGAAA | | |
| | | | CCCAACTGCGCGAACACGTCATTGATTGGGTAATCAAA | | |
| | | | GACGCAGGCATTGAGGATCTGGCAACCGGTGAGATCAC | | |
| | | | CGTGTTGATCAACCCTTCAGGTTCCTTCATTCTGGGTG | | |
| | | | GCCCCATGGGTGATGCGGGTCTGACCGGCCGCAAGATC | | |
| | | | ATCGTGGATACCTACGGTGGCATGGCTCGCCATGGTGG | | |
| | | | TGGAGCATTCTCCGGTAAGGATCCAAGCAAGGTGGACC | | |
| | | | GCTCTGCTGCATACGCCATGCGTTGGGTAGCAAAGAAC | | |
| | | | ATCGTGGCAGCAGGCCTTGCTGATCGCGCTGAAGTTCA | | |
| | | | GGTTGCATACGCCATTGGACGCGCAAAGCCAGTCGGAC | | |
| | | | TTTACGTTGAAACCTTTGACACCAACAAGGAAGGCCTG | | |
| | | | AGCGACGAGCAGATTCAGGCTGCCGTGTTGGAGGTCTT | | |
| | | | TGACCTGCGTCCAGCAGCAATTATCCGTGAGCTTGATC | | |
| | | | TGCTTCGTCCGATCTACGCTGACACTGCTGCCTACGGC | | |
| | | | CACTTTGGTCGCACTGATTTGGACCTTCCTTGGGAGGC | | |
| | | | TATCGACCGCGTTGATGAACTTCGCGCAGCCCTCAAGT | | |
| | | | TGGCC | | |
| metK | Escherichia | U28377 | ATGGCAAAACACCTTTTTACGTCCGAGTCCGTCTCTGA | 254 | |
| | coli | | AGGGCATCCTGACAAAATTGCTGACCAAATTTCTGATG | | |
| | | | CCGTTTTAGACGCGATCCTCGAACAGGATCCGAAAGCA | | |
| | | | CGCGTTGCTTGCGAAACCTACGTAAAAACCGGCATGGT | | |
| | | | TTTAGTTGGCGGCGAAATCACCACCAGCGCCTGGGTAG | | |
| | | | ACATCGAAGAGATCACCCGTAACACCGTTCGCGAAATT | | |
| | | | GGCTATGTGCATTCCGACATGGGCTTTGACGCTAACTC | | |
| | | | CTGTGCGGTTCTGAGCGCTATCGGCAAACAGTCTCCTG | | |
| | | | ACATCAACCAGGGCGTTGACCGTGCCGATCCGCTGGAA | | |
| | | | CAGGGCGCGGGTGACCAGGGTCTGATGTTTGGCTACGC | | |
| | | | AACTAATGAAACCGACGTGCTGATGCCAGCACCTATCA | | |
| | | | CCTATGCACACCGTCTGGTACAGCGTCAGGCTGAAGTG | | |
| | | | CGTAAAAACGGCACTCTGCCGTGGCTGCGCCCGGACGC | | |
| | | | GAAAAGCCAGGTGACTTTTCAGTATGACGACGGCAAAA | | |
| | | | TCGTTGGTATCGATGCTGTCGTGCTTTCCACTCAGCAC | | |
| | | | TCTGAAGAGATCGACCAGAAATCGCTGCAAGAAGCGGT | | |
| | | | AATGGAAGAGATCATCAAGCCAATTCTGCCCGCTGAAT | | |
| | | | GGCTGACTTCTGCCACCAAATTCTTCATCAACCCGACC | | |
| | | | GGTCGTTTCGTTATCGGTGGCCCAATGGGTGACTGCGG | | |
| | | | TCTGACTGGTCGTAAAATTATCGTTGATACCTACGGCG | | |
| | | | GCATGGCGCGTCACGGTGGCGGTGCATTCTCTGGTAAA | | |
| | | | GATCCATCAAAAGTGGACCGTTCCGCAGCCTACGCAGC | | |
| | | | ACGTTATGTCGCGAAAAACATCGTTGCTGCTGGCCTGG | | |
| | | | CCGATCGTTGTGAAATTCAGGTTTCCTACGCAATCGGC | | |
| | | | GTGGCTGAACCGACCTCCATCATGGTAGAAACTTTCGG | | |
| | | | TACTGAGAAAGTGCCTTCTGAACAACTGACCCTGCTGG | | |
| | | | TACGTGAGTTCTTCGACCTGCGCCCATACGGTCTGATT | | |
| | | | CAGATGCTGGATCTGCTGCACCCGATCTACAAAGAAAC | | |
| | | | CGCAGCATACGGTCACTTTGGTCGTGAACATTTCCCGT | | |
| | | | GGGAAAAAACCGACAAAGCGCAGCTGCTGCGCGATGCT | | |
| | | | GCCGGTCTGAAG | | |
| metC | Mycobacterium | AL021428.1 | ATGCAGGACAGCATCTTCAATCTGTTGACCGAGGAACA | 130 | |
| | tuberculosis | | GCTTCGGGGTCGCAACACGCTCAAGTGGAACTATTTCG | | |
| | (use this to | | GGCCCGATGTAGTGCCACTGTGGCTGGCGGAGATGGAC | | |
| | clone M. | | TTTCCCACCGCACCGGCTGTGCTCGACGGGGTGCGGGC | | |
| | smegmatis | | GTGCGTCGACAACGAGGAGTTCGGCTACCCGCCGTTGG | | |
| | gene) | | GCGAGGACAGCCTGCCGAGGGCGACGGCCGATTGGTGC | | |
| | | | CGACAACGCTACGGTTGGTGCCCCCGACCGGACTGGGT | | |
| | | | CCGCGTCGTGCCGGATGTCCTGAAGGGGATGGAAGTCG | | |
| | | | TCGTCGAATTCCTTACCCGGCCGGAGAGTCCGGTCGCG | | |
| | | | TTGCCGGTTCCGGCTTACATGCCGTTTTTCGACGTCCT | | |
| | | | GCACGTCACCGGCCGCCAACGAGTGGAAGTCCCAATGG | | |
| | | | TGCAGCAAGACTCGGGACGCTACCTGCTGGACCTGGAC | | |
| | | | GCTCTGCAGGCCGCGTTCGTCCGCGGTGCCGGATCGGT | | |
| | | | GATTATCTGCAATCCGAATAACCCACTGGGTACGGCGT | | |
| | | | TCACCGAAGCCGAGCTACGTGCGATTGTGGATATCGCG | | |
| | | | GCCCGCCACGGCGCCCGGGTGATCGCGGATGAGATCTG | | |
| | | | GGCACCGGTGGTCTACGGATCGCGCCATGTCGCCGCCG | | |
| | | | CTTCGGTGTCGGAGGCGGCGGCTGAAGTCGTGGTCACG | | |
| | | | TTGGTGTCGGCGTCCAAAGGCTGGAACTTGCCGGGTCT | | |
| | | | GATGTGCGCTCAGGTGATCCTGTCTAACCGCCGTGACG | | |
| | | | CCCACGACTGGGACCGGATCAACATGTTGCACCGCATG | | |
| | | | GGCGCATCAACGGTCGGTATCCGCGCGAACATCGCCGC | | |
| | | | CTACCATCATGGCGAATCTTGGTTGGACGAGCTGCTCC | | |
| | | | CTTATCTGCGGGCGAACCGTGATCATCTGGCACGGGCG | | |
| | | | CTGCCGGAGTTAGCTCCCGGGGTAGAGGTCAACGCTCC | | |
| | | | GGACGGTACCTACCTGTCGTGGGTGGATTTCCGTGCGC | | |
| | | | TGGCTCTGCCGTCTGAACCGGCGGAATACCTGCTCTCG | | |
| | | | AAGGCGAAGGTGGCGCTGTCGCCTGGCATTCCGTTCGG | | |
| | | | CGCCGCGGTGGGCTCGGGATTTGCGCGGCTGAACTTCG | | |
| | | | CCACCACCCGCGCAATACTGGATCGGGCGATCGAGGCT | | |
| | | | ATCGCGGCCGCCCTGCGCGACATCATCGATTAA | | |
| metC | Bifidobacterium | NZ_AABM020 | ATGAGCATGAACAACATTCCCCAGTCAACGACTGTGAG | 131 | |
| | longum | 00009.1 | CAACGCAACCGCCGACGTCTCTTGCTTTGATGCCAATC | | |
| | | | ACATCGACGTGACGACCATCGAGGATCTGAAGCAGGTC | | |
| | | | GGTTCGGATAAATGGACCCGCTACCCCGGCTGCATCGG | | |
| | | | CGCATTCATCGCCGAGATGGATTACGGTCTGGCACCAT | | |
| | | | GCGTGGCCGAAGCCATCGAAGAGGCCACCGAACGTGGC | | |
| | | | GCGCTCGGCTACATTCCCGACCCGTGGAAGAAGGAGGT | | |
| | | | CGCCCGCTCGTGCGCCGCATGGCAGCGCCGCTACGGCT | | |
| | | | GGGATGTGGATCCGACGTGCATCCGCCCGGTGCCGGAC | | |
| | | | GTGCTGGAGGCGTTCGAAGTGTTCCTGCGCGAGATCGT | | |
| | | | GCGCGCCGGCAACTCCATCGTGGTACCGACTCCGGCCT | | |
| | | | ATATGCCGTTCCTGAGCGTGCCGCGTCTGTATGGCGTG | | |
| | | | GAGGTCCTTGAGATTCCGATGCTGTGCGCGGGCGCCAG | | |
| | | | CGAGAGCAGCGGGCGCAATGATGAATGGCTGTTCGATT | | |
| | | | TCGACGCCATTGAGCAGGCGTTCGCGAACGGCTGCCAT | | |
| | | | GCCTTCGTGCTGTGCAACCCGCACAACCCGATCGGCAA | | |
| | | | GGTATTGACGCGCGAGGAAATGCTGCGATTGTCCGATC | | |
| | | | TGGCCGCCAAGTACAACGTGCGTATATTCTCCGATGAG | | |
| | | | ATTCACGCGCCGTTCGTCTACCAAGGCCACACGCATGT | | |
| | | | GCCATTCGCCTCAATCAACCGGCAGACGGCCATGCAGG | | |
| | | | CTTTCACCTCCACTTCAGCCTCGAAGTCGTTCAACATT | | |
| | | | CCCGGCACCAAGTGCGCGCAGGTGATTCTCACCAATCC | | |
| | | | GGACGATCTGGAACTATGGATGAGGAACGCGGAATGGT | | |
| | | | CCGAGCACCAGACGGCCACCATCGGTGCCATAGCCACC | | |
| | | | ACTGCGGCCTATGACGGCGGCGCGGCATGGTTCGAGGG | | |
| | | | CGTGATGGCATATATCGAGCGCAATATCGCGCTGGTCA | | |
| | | | ACGAGCAGATGCGCACGAGATTCGCCAAGGTGCGCTAT | | |
| | | | GTGGAGCCGCAGGGCACGTATATCGCGTGGCTGGATTT | | |
| | | | CTCGCCACTGGGCATCGGCGACCCGGCCAACTATTTCT | | |
| | | | TTAAGAAGGCCAACGTGGCGTTGACAGACGGCCGTGAA | | |
| | | | TGCGGCGAGGTCGGGCGCGGTTGCGTGCGTATGAACTT | | |
| | | | CGCCATGCCCTACCCGCTACTGGAGGAATGCTTCGACC | | |
| | | | GCATGGCCGCCGCACTTGAGGCGGACGGGTTGTTGTAG | | |
| metC | Lactobacillus | L935262 | ATGCAATATGATTTTAATAAGGTTATAAATCGTAGAGG | 132 | |
| | plantarum | | GACATACAGTACTCAGTGGGATTATATTCAAGATCGCT | | |
| | | | TTGGTCGTTCTGACATTCTACCATTTTCAATTTCAGAT | | |
| | | | ACTGACTTTCCGGTTCCCGTTGGCGTCCAAGAGGCGCT | | |
| | | | TGAACAGCGTATTAAGCATCCTATTTATGGTTATACAC | | |
| | | | GCTGGAATAATGAGGATTACAAAAATAGTATTATTAAT | | |
| | | | TGGTTTAGCTCTCAAAATCAAGTTACTATAAACCCAGA | | |
| | | | TTGGATTTTATATAGTCCCAGTGTTGTTTTTTCAATTG | | |
| | | | CCACCTTTATTCGAATGAAGTCAGCCGTTGGAGAAAGT | | |
| | | | GTAGCGGTCTTCACTCCTATGTATGACGCCTTTTATCA | | |
| | | | TGTGATTGAGGATAATCAGCGGGTGTTAGCGCCGGTCA | | |
| | | | GACTAGGCAGTGCACAACAAGACTATAGTATCGATTGG | | |
| | | | GATACTTTGAAAGCTGTTTTAAAGCAAACAGCAACAAA | | |
| | | | AATTTTACTTTTGACTAATCCACATAATCCTACCGGGA | | |
| | | | AGGTCTTTTCAGATGATGAATTGAAGCATATAGTTGCA | | |
| | | | CTATGTCAACAATATAATGTCTTTATAATTTCAGATGA | | |
| | | | TATTCATAAGGACATTGTGTATCAAAAGGCAGCATATA | | |
| | | | CGCCTGTAACCGAATTTACAACTAAGAATGTGGTCCTA | | |
| | | | TGTTGTTCAGCTACTAAAACTTTTAATACCCCTGGGTT | | |
| | | | GATTGGCGCATATTTATTTGAGCCTGAGGCTGAACTAC | | |
| | | | GTGAGATGTTTTTATGTGAATTAAAGCAAAAAAATGCT | | |
| | | | TTATCATCAGCTAGCATCCTTGGAATTGAATCTCAGAT | | |
| | | | GGCTGCTTATAATACTGGAAGTGACTATTTAGTACAAC | | |
| | | | TCATAACGTATTTGCAAAATAACTTTGATTATCTATCT | | |
| | | | ACTTTCTTAAAAAGTCAGTTACCAGAGATTAGATTTAA | | |
| | | | GCAGCCTGAAGCGACTTATTTGGCTTGGATGGATGTCT | | |
| | | | CGCAATTGGGGCTAACGGCTGAAAAACTACAAGATAAA | | |
| | | | CTTGTTAATACGGGTCGAGTTGGGATCATGTCGGGGAC | | |
| | | | AACATATGGTGACAGTCATTATTTACGTATGAATATTG | | |
| | | | CTTGTCCTATTTCTAAATTGCAGGAAGGACTGAAAAGA | | |
| | | | ATGGAGTACGGGATCCGTTCGTAA | | |
| metC | Coryne- | F276227 | ATGCGATTTCCTGAACTCGAAGAATTGAAGAATCGCCG | 255 | |
| | bacterium | | GACCTTGAAATGGACCCGGTTTCCAGAAGACGTGCTTC | | |
| | glutamicum | | CTTTGTGGGTTGCGGAAAGTGATTTTGGCACCTGCCCG | | |
| | | | CAGTTGAAGGAAGCTATGGCAGATGCCGTTGAGCGCGA | | |
| | | | GGTCTTCGGATACCCACCAGATGCTACTGGGTTGAATG | | |
| | | | ATGCGTTGACTGGATTCTACGAGCGTCGCTATGGGTTT | | |
| | | | GGCCCAAATCCGGAAAGTGTTTTCGCCATTCCGGATGT | | |
| | | | GGTTCGTGGCCTGAAGCTTGCCATTGAGCATTTCACTA | | |
| | | | AGCCTGGTTCGGCGATCATTGTGCCGTTGCCTGCATAC | | |
| | | | CCTCCTTTCATTGAGTTGCCTAAGGTGACTGGTCGTCA | | |
| | | | GGCGATCTACATTGATGCGCATGAGTACGATTTGAAGG | | |
| | | | AAATTGAGAAGGCCTTCGCTGACGGTGCGGGATCACTG | | |
| | | | TTGTTCTGCAATCCACACAACCCACTGGGCACGGTCTT | | |
| | | | TTCTGAAGAGTACATCCGCGAGCTCACCGATATTGCGG | | |
| | | | CGAAGTACGATGCCCGCATCATCGTCGATGAGATCCAC | | |
| | | | GCGCCACTGGTTTATGAAGGCACCCATGTGGTTGCTGC | | |
| | | | TGGTGTTTCTGAGAACGCTGCAAACACTTGCATCACCA | | |
| | | | TCACCGCAACTTCTAAGGCGTGGAACACTGCTGGTTTG | | |
| | | | AAGTGTGCTCAGATCTTCTTCAGTAATGAAGCCGATGT | | |
| | | | GAAGGCCTGGAAGAATTTGTCGGATATTACCCGTGACG | | |
| | | | GTGTGTCCATCCTTGGATTGATCGCTGCGGAGACAGTG | | |
| | | | TACAACGAGGGCGAAGAATTCCTTGATGAGTCAATTCA | | |
| | | | GATTCTCAAGGACAACCGTGACTTTGCGGCTGCTGAAC | | |
| | | | TGGAAAAGCTTGGCGTGAAGGTCTACGCACCGGACTCC | | |
| | | | ACTTATTTGATGTGGTTGGACTTCGCTGGCACCAAGAT | | |
| | | | CGAAGAGGCGCCTTCTAAAATTCTTCGTGAGGAGGGTA | | |
| | | | AGGTCATGCTGAATGATGGCGCAGCTTTTGGTGGTTTC | | |
| | | | ACCACCTGCGCTCGTCTTAATTTTGCGTGTTCCAGAGA | | |
| | | | GACCCTTGAGGAGGGGCTGCGCCGTATCGCCAGCGTGT | | |
| | | | TGTAA | | |
| metC | Escherichia coli | E000383 | ATGGCGGACAAAAAGCTTGATACTCAACTGGTGAATGC | 256 | |
| | | | AGGACGCAGCAAAAAATACACTCTCGGCGCGGTAAATA | | |
| | | | GCGTGATTCAGCGCGCTTCTTCGCTGGTCTTTGACAGT | | |
| | | | GTAGAAGCCAAAAAACACGCGACACGTAATCGCGCCAA | | |
| | | | TGGAGAGTTGTTCTATGGACGGCGCGGAACGTTAACCC | | |
| | | | ATTTCTCCTTACAACAAGCGATGTGTGAACTGGAAGGT | | |
| | | | GGCGCAGGCTGCGTGCTATTTCCCTGCGGGGCGGCAGC | | |
| | | | GGTTGCTAATTCCATTCTTGCTTTTATCGAACAGGGCG | | |
| | | | ATCATGTGTTGATGACCAACACCGCCTATGAACCGAGT | | |
| | | | CAGGATTTCTGTAGCAAAATCCTCAGCAAACTGGGCGT | | |
| | | | AACGACATCATGGTTTGATCCGCTGATTGGTGCCGATA | | |
| | | | TCGTTAAGCATCTGCAGCCAAACACTAAAATCGTGTTT | | |
| | | | CTGGAATCGCCAGGCTCCATCACCATGGAAGTCCACGA | | |
| | | | CGTTCCGGCGATTGTTGCCGCCGTACGCAGTGTGGTGC | | |
| | | | CGGATGCCATCATTATGATCGACAACACCTGGGCAGCC | | |
| | | | GGTGTGCTGTTTAAGGCGCTGGATTTTGGCATCGATGT | | |
| | | | TTCTATTCAAGCCGCCACCAAATATCTGGTTGGGCATT | | |
| | | | CAGATGCGATGATTGGCACTGCCGTGTGCAATGCCCGT | | |
| | | | TGCTGGGAGCAGCTACGGGAAAATGCCTATCTGATGGG | | |
| | | | CCAGATGGTCGATGCCGATACCGCCTATATAACCAGCC | | |
| | | | GTGGCCTGCGCACATTAGGTGTGCGTTTGCGTCAACAT | | |
| | | | CATGAAAGCAGTCTGAAAGTGGCTGAATGGCTGGCAGA | | |
| | | | ACATCCGCAAGTTGCGCGAGTTAACCACCCTGCTCTGC | | |
| | | | CTGGCAGTAAAGGTCACGAATTCTGGAAACGAGACTTT | | |
| | | | ACAGGCAGCAGCGGGCTATTTTCCTTTGTGCTTAAGAA | | |
| | | | AAAACTCAATAATGAAGAGCTGGCGAACTATCTGGATA | | |
| | | | ACTTCAGTTTATTCAGCATGGCCTACTCGTGGGGCGGG | | |
| | | | TATGAATCGTTGATCCTGGCAAATCAACCAGAACATAT | | |
| | | | CGCCGCCATTCGCCCACAAGGCGAGATCGATTTTAGCG | | |
| | | | GGACCTTGATTCGCCTGCATATTGGTCTGGAAGATGTC | | |
| | | | GACGATCTGATTGCCGATCTGGACGCCGGTTTTGCGCG | | |
| | | | AATTGTA | | |
| gdh | Streptomyces | L939121.1 | GTGCCCGCCGTGCCAGAAAGGGCCCCTGTGACGACGCG | 133 | |
| | coelicolor | | AAGCGAGACGCAGTCCACCCTCGACCACCTCCTCACCG | | |
| | | | AGATCGAGCTGCGCAACCCGGCCCAGCCCGAGTTCCAC | | |
| | | | CAGGCGGCCCACGAGGTCCTGGAGACCCTGGCGCCGGT | | |
| | | | CGTCGCGGCCCGCCCCGAGTACGCCGAGCCGGGCCTCA | | |
| | | | TCGAGCGGCTGGTCGAGCCGGAGCGCCAGGTGATGTTC | | |
| | | | CGGGTGCCGTGGCAGGACGACCAGGGCCGCGTCCGCGT | | |
| | | | CAACCGGGGCTTCCGGGTCGAGTTCAACAGCGCGCTGG | | |
| | | | GCCCGTACAAGGGCGGTCTGCGCTTCCATCCGTCCGTC | | |
| | | | AACCTGGGCGTCATCAAGTTCCTGGGCTTCGAGCAGAT | | |
| | | | CTTCAAGAACGCGCTGACCGGCCTCGGCATCGGCGGCG | | |
| | | | GCAAGGGCGGCAGCGACTTCGACCCGCACGGGCGCAGC | | |
| | | | GACGCGGAGGTCATGCGGTTCTGCCAGTCCTTCATGAC | | |
| | | | GGAGCTGTACCGGCACATCGGCGAGCACACGGACGTCC | | |
| | | | CGGCGGGGGACATCGGCGTCGGGGGCCGCGAGATCGGC | | |
| | | | TACCTCTTCGGCCAGTACCGGCGGATCACCAACCGCTG | | |
| | | | GGAGTCCGGCGTCCTGACCGGCAAGGGCCAGGGCTGGG | | |
| | | | GCGGCTCGCTGATCCGCCCGGAGGCGACCGGCTACGGC | | |
| | | | AACGTGCTGTTCGCGGCGGCGATGCTGCGGGAGCGCGG | | |
| | | | CGAGGACCTGGAGGGCCAGACCGCGGTCGTCTCCGGCT | | |
| | | | CCGGCAACGTGGCGATCTACACCATCGAGAAGCTGACC | | |
| | | | GCCCTCGGCGCCAACGCCGTCACCTGCTCGGACTCCTC | | |
| | | | CGGCTACGTCGTCGACGAGAAGGGCATCGACCTCGACC | | |
| | | | TGCTCAAGCAGATCAAGGAGGTCGAGCGCGGCCGCGTC | | |
| | | | GACGCGTACGCCGAGCGCCGGGGCGCCTCGGCCCGCTT | | |
| | | | CGTGCCCGGCGGCAGCGTCTGGGACGTTCCGGCCGACC | | |
| | | | TTGCCCTCCCCTCCGCCACGCAGAACGAGCTGGACGAG | | |
| | | | AACGCCGCCGCCACGCTCGTCCGCAACGGCGTCAAGGC | | |
| | | | GGTCTCCGAGGGCGCGAACATGCCGACCACCCCCGAGG | | |
| | | | CCGTCCACCTGCTCCAGAAGGCGGGCGTCGCCTTCGGC | | |
| | | | CCCGGCAAGGCGGCCAACGCGGGCGGCGTCGCGGTCAG | | |
| | | | CGCCCTGGAGATGGCGCAGAACCACGCCCGTACCTCGT | | |
| | | | GGACGGCGGCGCGGGTCGAGGAGGAGCTGGCCGACATC | | |
| | | | ATGACCAGCATCCACACCACCTGCCACGAGACCGCCGA | | |
| | | | GCGCTACGACGCCCCCGGCGACTACGTCACCGGCGCGA | | |
| | | | ACATCGCCGGCTTCGAGCGGGTGGCCGACGCGATGCTG | | |
| | | | GCGCAGGGCGTCATCTGA | | |
| gdh | Thermobifida | NZ_AAAQ010 | GTGCGCCCCGAACCGGAGGCGACCATGTCGGCGAATCT | 134 | |
| | fusca | 00033.1 | CGATGAGAAACTGTCCCCGATCTACGAGGAAATCCTGC | | |
| | | | GGCGTAACCCGGGGGAGGTCGAGTTCCACCAGGCTGTT | | |
| | | | CGCGAAGTCCTGGAGTGCCTCGGCCCCGTGGTGGCCAA | | |
| | | | GAACCCTGACATCAGCCACGCCAAGATCATCGAGCGGC | | |
| | | | TCTGTGAGCCGGAGCGCCAGCTGATCTTCCGGGTGCCC | | |
| | | | TGGATGGACGACTCCGGTGAGATCCACGTCAACCGGGG | | |
| | | | TTTCCGGGTGGAGTTCAGCAGCTCTTTGGGACCTTACA | | |
| | | | AGGGCGGGCTGCGGTTCCACCCGTCGGTGAACCTGAGC | | |
| | | | ATCATCAAGTTCCTCGGGTTCGAGCAGATCTTCAAGAA | | |
| | | | CTCGCTGACCGGATTGCCGATCGGCGGTGCGAAAGGCG | | |
| | | | GCAGCGACTTCGACCCGAAGGGCCGTTCCGACGCCGAG | | |
| | | | ATCATGCGGTTCTGCCAGTCGTTCATGACGGAGCTGTA | | |
| | | | CCGGCACCTGGGTGAGCACACGGACGTGCCTGCCGGTG | | |
| | | | ACATCGGCGTGGGCCAGCGTGAGATCGGCTACCTGTTC | | |
| | | | GGCCAGTACAAGCGGATCACCAACCGCTACGAGTCGGG | | |
| | | | CGTGTTCACCGGTAAGGGCCTCAGTTGGGGCGGTTCCC | | |
| | | | AGGTGCGTCGTGAGGCCACCGGGTACGGCTGTGTGCTC | | |
| | | | TTCACTGCGGAGATGCTGCGAGCCCGCGGCGACTCGCT | | |
| | | | GGAAGGCAAGCGGGTCTCGGTGTCGGGTTCGGGCAATG | | |
| | | | TGGCGATCTACGCGATCGAGAAGGCCCAGCAGCTCGGC | | |
| | | | GCGCATGTGGTGACCTGCTCGGACTCCAACGGCTACGT | | |
| | | | GGTGGACGAGAAGGGGATCGACCTGGAGCTGCTCAAGC | | |
| | | | AGGTCAAGGAGGTCGAACGCGGCCGGGTGTCCGACTAC | | |
| | | | GCCAAGCGGCGCGGCTCCCACGTCCGCTACATCGACTC | | |
| | | | GTCGTCGTCCAGCGTGTGGGAGGTGCCCTGCGACATCG | | |
| | | | CGCTGCCGTGCGCGACGCAGAACGAGCTGACCGGCCGC | | |
| | | | GACGCTATCACCCTGGTGCGCAACGGGGTGGGCGCGGT | | |
| | | | GGCGGAGGGCGCGAACATGCCCACGACCCCGGAGGGGA | | |
| | | | TCCGGGTGTTCGCGGAGGCGGGCGTAGCGTTCGCGCCG | | |
| | | | GGCAAGGCCGCGAACGCGGGCGGGGTGGCGACGAGCGC | | |
| | | | GTTGGAGATGCAGCAGAACGCGTCCCGCGACTCGTGGT | | |
| | | | CGTTCGAGTACACCGAGAAGCGGCTCGCGGAAATCATG | | |
| | | | CGCCACATCCACGACACCTGCTATGAGACGGCGGAACG | | |
| | | | CTATGGGCGGCCCGGCGACTATGTGGCAGGTGCCAACA | | |
| | | | TCGCTGCTTTCGAGATCGTCGCTGAGGCGATGCTCGCT | | |
| | | | CAGGGCCTGATCTGA | | |
| gdh | Lactobacillus | AL935255.1 | TTGAGTCAAGCAACCGATTATGTCCAACATGTTTACCA | 135 | |
| | plantarum | | AGTCATTGAACACCGTGATCCGAACCAAACCGAATTTT | | |
| | | | TAGAGGCCATCAACGACGTCTTCAAAACGATCACGCCA | | |
| | | | GTCCTCGAACAACATCCAGAATATATCGAAGCCAATAT | | |
| | | | TTTGGAACGTTTGACCGAACCAGAACGGATTATTCAAT | | |
| | | | TCCGGGTTCCTTGGCTCGACGATGCTGGTCATGCACGA | | |
| | | | GTCAACCGTGGGTTCCGAGTACAATTTAACTCAGCAAT | | |
| | | | CGGTCCTTACAAGGGCGGCTTACGGTTACACCCATCCG | | |
| | | | TTAATCTGAGTATCGTCAAATTCTTGGGCTTTGAACAG | | |
| | | | ATCTTCAAAAATGCCCTGACCGGCCTACCAATTGGCGG | | |
| | | | TGGTAAAGGGGGCTCTGATTTCGACCCTAAGGGCAAAT | | |
| | | | CAGACAACGAAATTATGCGCTTCTGTCAGAGTTTCATG | | |
| | | | ACCGAACTGAGCAAGTACATTGGTCTCGATACTGACGT | | |
| | | | TCCTGCTGGTGATATCGGTGTTGGTGGCCGCGAAATCG | | |
| | | | GCTTTTTATACGGCCAATACAAGCGACTCCGGGGCGCT | | |
| | | | GACCGCGGCGTACTCACCGGTAAAGGATTGAACTATGG | | |
| | | | CGGTTCGTTAGCCCGGACTGAAGCTACCGGTTATGGTC | | |
| | | | TCGCCTACTATACCAACGAAATGCTCAAGGCCAACCAA | | |
| | | | CTTTCCTTCCCTGGTCAACGCGTTGCCATTTCTGGTGC | | |
| | | | TGGTAATGTCGCCATCTACGCGATTCAAAAGGTTGAAG | | |
| | | | AACTCGGTGGCAAGGTGATTACTTGCTCCGACTCAAAC | | |
| | | | GGTTACGTTATTGACGAAAACGGTATCGACTTCAAGAT | | |
| | | | CGTTAAGCAGATCAAGGAAGTTGAACGCGGTCGTATCA | | |
| | | | AAGACTATGCCGACCGTGTAGCCAGTGCCAGCTATTAC | | |
| | | | GAAGGTTCCGTCTGGGACGCCCAAGTAGCTTATGATAT | | |
| | | | CGCGTTACCTTGCGCCACCCAAAACGAAATCAGCGGTG | | |
| | | | ATCAAGCCAAGAACTTGATTGCCAATGGTGCCAAGGTC | | |
| | | | GTTGCCGAAGGGGCTAACATGCCTAGCAGTCCAGAAGC | | |
| | | | CATTGCGACATACCAAGCTGCCAGCTTGCTATATGGTC | | |
| | | | CGGCCAAAGCTGCCAATGCTGGTGGCGTTGCCGTTTCC | | |
| | | | GCCCTTGAAATGAGCCAAAATAGTATGCGTTTGAGCTG | | |
| | | | GACTTTTGAAGAAGTCGATAATCGCCTCAAGCAAATCA | | |
| | | | TGCAAGATATCTTTGCACACTCCGTTGCCGCTGCCGAC | | |
| | | | GAATACCACGTTAGCGGTGATTACCTGAGTGGTGCTAA | | |
| | | | CATTGCTGGCTTCACAAAAGTTGCTGACGCCATGTTAG | | |
| | | | CGCAAGGCTTAGTTTAA | | |
| gdh | Corynebacterium | X59404 | ATGACAGTTGATGAGCAGGTCTCTAACTATTACGACAT | 257 | |
| | glutamicum | | GCTTCTGAAGCGCAATGCTGGCGAGCCTGAATTTCACC | | |
| | | | AGGCAGTGGCAGAGGTTTTGGAATCTTTGAAGCTCGTC | | |
| | | | CTGGAAAAGGACCCTCATTACGCTGATTACGGTCTCAT | | |
| | | | CCAGCGCCTGTGCGAGCCTGAGCGTCAGCTCATCTTCC | | |
| | | | GTGTGCCTTGGGTTGATGACCAGGGCCAGGTCCACGTC | | |
| | | | AACCGTGGTTTCCGCGTGCAGTTCAACTCTGCACTTGG | | |
| | | | ACCATACAAGGGCGGCCTGCGCTTCCACCCATCTGTAA | | |
| | | | ACCTGGGCATTGTGAAGTTCCTGGGCTTTGAGCAGATC | | |
| | | | TTTAAAAACTCCCTAACCGGCCTGCCAATCGGTGGTGG | | |
| | | | CAAGGGTGGATCCGACTTCGACCCTAAGGGCAAGTCCG | | |
| | | | ATCTGGAAATCATGCGTTTCTGCCAGTCCTTCATGACC | | |
| | | | GAGCTACACCGCCACATCGGTGAGTACCGCGACGTTCC | | |
| | | | TGCAGGTGACATCGGAGTTGGTGGCCGCGAGATCGGTT | | |
| | | | ACCTGTTTGGCCACTACCGTCGCATGGCTAACCAGCAC | | |
| | | | GAGTCCGGCGTTTTGACCGGTAAGGGCCTGACCTGGGG | | |
| | | | TGGATCCCTGGTCCGCACCGAGGCAACTGGCTACGGCT | | |
| | | | GCGTTTACTTCGTGAGTGAAATGATCAAGGCTAAGGGC | | |
| | | | GAGAGCATCAGCGGCCAGAAGATCATCGTTTCCGGTTC | | |
| | | | CGGCAACGTAGCAACCTACGCGATTGAAAAGGCTCAGG | | |
| | | | AACTCGGCGCAACCGTTATTGGTTTCTCCGATTCCAGC | | |
| | | | GGTTGGGTTCATACCCCTAACGGCGTTGACGTGGCTAA | | |
| | | | GCTCCGCGAAATCAAGGAAGTTCGTCGCGCACGCGTAT | | |
| | | | CCGTGTACGCCGACGAAGTTGAAGGCGCAACCTACCAC | | |
| | | | ACCGACGGTTCCATCTGGGATCTCAAGTGCGATATCGC | | |
| | | | TCTTCCTTGTGCAACTCAGAACGAGCTCAACGGCGAGA | | |
| | | | ACGCTAAGACTCTTGCAGACAACGGCTGCCGTTTCGTT | | |
| | | | GCTGAAGGCGCGAACATGCCTTCCACCCCTGAGGCTGT | | |
| | | | TGAGGTCTTCCGTGAGCGCGACATCCGCTTCGGACCAG | | |
| | | | GCAAGGCCACCCCTGAGGCTGTTGAGGTCTTCCGTGAG | | |
| | | | CGCGACATCCGCTTCGGACCAGGCAAGGCAGTCAACGT | | |
| | | | CGGTGGCGTTGCAACCTCCGCTCTGGAGATGCAGCAGA | | |
| | | | ACGCTTCGCGCGAGACCTGTGCAGAGACCGCAGCAGAG | | |
| | | | TATGGACACGAGAACGATTACGTTGTCGGCGCTAACAT | | |
| | | | TGCTGGCTTCAAGAAGGTAGCTGACGCGATGCTGGCAC | | |
| | | | AGGGCGTCATCTAA | | |
| gdh | Escherichia coli | D90819 | ATGGATCAGACATATTCTCTGGAGTCATTCCTCAACCA | 258 | |
| | | | TGTCCAAAAGCGCGACCCGAATCAAACCGAGTTCGCGC | | |
| | | | AAGCCGTTCGTGAAGTAATGACCACACTCTGGCCTTTT | | |
| | | | CTTGAACAAAATCCAAAATATCGCCAGATGTCATTACT | | |
| | | | GGAGCGTCTGGTTGAACCGGAGCGCGTGATCCAGTTTC | | |
| | | | GCGTGGTATGGGTTGATGATCGCAACCAGATACAGGTC | | |
| | | | AACCGTGCATGGCGTGTGCAGTTCAGCTCTGCCATCGG | | |
| | | | CCCGTACAAAGGCGGTATGCGCTTCCATCCGTCAGTTA | | |
| | | | ACCTTTCCATTCTCAAATTCCTCGGCTTTGAACAAACC | | |
| | | | TTCAAAAATGCCCTGACTACTCTGCCGATGGGCGGTGG | | |
| | | | TAAAGGCGGCAGCGATTTCGATCCGAAAGGAAAAAGCG | | |
| | | | AAGGTGAAGTGATGCGTTTTTGCCAGGCGCTGATGACT | | |
| | | | GAACTGTATCGCCACCTGGGCGCGGATACCGACGTTCC | | |
| | | | GGCAGGTGATATCGGGGTTGGTGGTCGTGAAGTCGGCT | | |
| | | | TTATGGCGGGGATGATGAAAAAGCTCTCCAACAATACC | | |
| | | | GCCTGCGTCTTCACCGGTAAGGGCCTTTCATTTGGCGG | | |
| | | | CAGTCTTATTCGCCCGGAAGCTACCGGCTACGGTCTGG | | |
| | | | TTTATTTCACAGAAGCAATGCTAAAACGCCACGGTATG | | |
| | | | GGTTTTGAAGGGATGCGCGTTTCCGTTTCTGGCTCCGG | | |
| | | | CAACGTCGCCCAGTACGCTATCGAAAAAGCGATGGAAT | | |
| | | | TTGGTGCTCGTGTGATCACTGCGTCAGACTCCAGCGGC | | |
| | | | ACTGTAGTTGATGAAAGCGGATTCACGAAAGAGAAACT | | |
| | | | GGCACGTCTTATCGAAATCAAAGCCAGCCGCGATGGTC | | |
| | | | GAGTGGCAGATTACGCCAAAGAATTTGGTCTGGTCTAT | | |
| | | | CTCGAAGGCCAACAGCCGTGGTCTCTACCGGTTGATAT | | |
| | | | CGCCCTGCCTTGCGCCACCCAGAATGAACTGGATGTTG | | |
| | | | ACGCCGCGCATCAGCTTATCGCTAATGGCGTTAAAGCC | | |
| | | | GTCGCCGAAGGGGCAAATATGCCGACCACCATCGAAGC | | |
| | | | GACTGAACTGTTCCAGCAGGCAGGCGTACTATTTGCAC | | |
| | | | CGGGTAAAGCGGCTAATGCTGGTGGCGTCGCTACATCG | | |
| | | | GGCCTGGAAATGGCACAAAACGCTGCGCGCCTGGGCTG | | |
| | | | GAAAGCCGAGAAAGTTGACGCACGTTTGCATCACATCA | | |
| | | | TGCTGGATATCCACCATGCCTGTGTTGAGCATGGTGGT | | |
| | | | GAAGGTGAGCAAACCAACTACGTGCAGGGCGCGAACAT | | |
| | | | TGCCGGTTTTGTGAAGGTTGCCGATGCGATGCTGGCGC | | |
| | | | AGGGTGTGATT | | |
| ddh | Bacillus | AB030649 | ATGAGTGCAATTCGAGTAGGTATTGTCGGTTATGGAAA | 136 | |
| | sphaericus | | TTTAGGGCGCGGTGTTGAATTCGCTATTTCACAAAATC | | |
| | | | CAGATATGGAATTAGTAGCGGTATTCACTCGTCGCGAT | | |
| | | | CCTTCAACAGTGAGCGTTGCAAGTAACGCGAGCGTATA | | |
| | | | TTTAGTAGATGATGCTGAAAAATTTCAAGATGACATTG | | |
| | | | ATGTAATGATTTTATGTGGTGGCTCTGCAACAGATTTA | | |
| | | | CCTGAGCAAGGTCCACACTTTGCGCAATGGTTTAATAC | | |
| | | | AATTGATAGTTTTGATACTCATGCGAAAATTCCAGAGT | | |
| | | | TTTTCGATGCGGTTGACGCTGCTGCTCAAAAATCTGGT | | |
| | | | AAAGTATCTGTTATCTCTGTAGGTTGGGATCCAGGTCT | | |
| | | | ATTTTCTTTAAATCGTGTTTTAGGCGAGGCAGTATTAC | | |
| | | | CTGTAGGTACAACGTATACATTCTGGGGTGATGGCTTA | | |
| | | | AGTCAAGGTCACTCGGATGCAGTTCGTCGTATTGAAGG | | |
| | | | GGTTAAAAATGCTGTACAGTATACATTACCTATCAAAG | | |
| | | | ATGCTGTTGAACGTGTTCGTAATGGTGAGAATCCAGAG | | |
| | | | CTTACTACACGTGAAAAGCATGCACGTGAATGCTGGGT | | |
| | | | AGTGCTTGAAGAAGGTGCAGATGCGCCAAAAGTAGAGC | | |
| | | | AAGAAATTGTAACAATGCCGAACTATTTCGATGAGTAT | | |
| | | | AACACAACTGTAAACTTTATCTCTGAAGATGAGTTTAA | | |
| | | | TGCCAACCATACAGGCATGCCACATGGTGGCTTCGTTA | | |
| | | | TTCGTAGTGGTGAAAGCGGCGCTAATGATAAACAAATT | | |
| | | | TTAGAATTCTCGTTAAAACTTGAAAGTAATCCAAACTT | | |
| | | | CACGTCAAGTGTCCTTGTGGCTTATGCACGTGCAGCAC | | |
| | | | ACCGCTTAAGTCAAGCGGGTGAAAAAGGTGCAAAAACA | | |
| | | | GTATTCGATATTCCGTTCGGTCTGTTATCTCCAAAATC | | |
| | | | AGCTGCACAATTACGTAAGGAACTATTATAA | | |
| dtsR1 | Thermobifida | NZ_AAAQ010 | ATGGCGACCCAAGCCCCTGAACCGCTGCCCGCGGACCA | 137 | |
| | fusca | 00037.1 | GATCGACATTCGCACCACCGCGGGCAAACTCGCAGACC | | |
| | | | TGCAGCGACGCCGCTACGAGGCGGTCCACGCAGGCTCC | | |
| | | | GAACGAGCCGTAGCAAAACAGCACGCCAAGGGCAAGAT | | |
| | | | GACCGCCCGCGAGCGCATCGACGCCCTGCTCGACCCGG | | |
| | | | GCTCCTTCGTGGAGTTCGACGCCTTCGCGCGTCACCGG | | |
| | | | TCCACCAACTTCGGCTTGGAGAAGAACCGCCCCTACGG | | |
| | | | CGACGGCGTCGTCACCGGCTACGGCACCATCGACGGCC | | |
| | | | GACCGGTCGCCGTGTTCAGCCAGGACGTCACCGTCTTC | | |
| | | | GGCGGTTCCCTCGGCGAGGTCTACGGCGAGAAGATCGT | | |
| | | | CAAAGTCCTCGACCATGCGCTCAAAACCGGCTGCCCGG | | |
| | | | TCATCGGCATCAACGAAGGCGGCGGCGCGCGCATCCAA | | |
| | | | GAGGGCGTGGTGGCGCTGGGCCTCTACGCCGAGATTTT | | |
| | | | CAAACGCAACACCCACGCCTCCGGGGTCATCCCCCAGA | | |
| | | | TCTCGCTCGTCATGGGGGCAGCAGCAGGCGGCCACGTC | | |
| | | | TACTCGCCCGCCCTCACCGACTTCATCGTCATGGTCGA | | |
| | | | CCAGACCTCCCAGATGTTCATCACCGGGCCCGACGTCA | | |
| | | | TCAAGACGGTCACCGGTGAAGACGTCACCATGGAGGAG | | |
| | | | CTGGGCGGCGCACGCACCCACAACACCAAGTCGGGCGT | | |
| | | | GGCCCACTACATGGCCTCCGACGAGCACGACGCCCTGG | | |
| | | | AGTACGTCAAGGCGCTGCTGTCCTACCTGCCCTCCAAC | | |
| | | | AACCTGGACGAGCCGCCCGTCGAACCCGTCCAGGTGAC | | |
| | | | CCTGGAGGTGACCGAGGAAGACCGGGAGCTGGACACCT | | |
| | | | TCATCCCCGACTCGGCCAACCAGCCCTACGACATGCGC | | |
| | | | CGCGTCATCGAACACATCGTGGACGACGGGGAGTTCCT | | |
| | | | GGAAGTCCACGAACTGTTCGCGCAGAACATCATCGTGG | | |
| | | | GCTTCGGCCGGGTCGAAGGCCACCCGGTAGGTGTCGTC | | |
| | | | GCCAACCAGCCGATGAACCTCGCGGGCTGCCTGGACAT | | |
| | | | CGACGCCTCCGAGAAAGCCGCCCGGTTCGTCCGCACCT | | |
| | | | GCGACGCCTTCAACATCCCCGTGCTGACCCTGGTCGAC | | |
| | | | GTCCCCGGCTTCCTGCCCGGAACCGACCAGGAGTTCGG | | |
| | | | CGGCATCATCCGGCGCGGCGCCAAACTGCTCTACGCCT | | |
| | | | ACGCTGAGGCGACCGTCCCCCTGGTGACCATCATCACC | | |
| | | | CGCAAAGCGTTCGGCGGCGCCTACGACGTCATGGGCTC | | |
| | | | CAAGCACCTGGGTGCAGACATCAACCTGGCGTGGCCGA | | |
| | | | CCGCGCAGATCGCGGTCATGGGAGCCCAGGGTGCCGTC | | |
| | | | AACATCCTGCACCGGCGTACCCTCGCCGCCGCCGACGA | | |
| | | | CGTCGAAGCGACCCGCGCCCAGCTCATCGCCGAATACG | | |
| | | | AAGACACTCTGCTCAACCCGTACAGCGCGGCCGAACGG | | |
| | | | GGCTACGTCGACAGCGTCATCATGCCGTCGGAAACCCG | | |
| | | | CACGTCCGTCATCAAAGCCCTGCGTGCGCTGCGCGGCA | | |
| | | | AACGCAAGCAGCTCCCGCCCAAGAAGCACGGGAATATC | | |
| | | | CCACTCTGA | | |
| dtsR1 | Streptomyces | AF113605.1 | ATGTCCGAGCCGGAAGAGCAGCAGCCCGACATCCACAC | 138 | |
| | coelicolor | | GACCGCGGGCAAGCTCGCGGATCTCAGGCGCCGTATCG | | |
| | | | AGGAAGCGACGCACGCCGGTTCCGCACGCGCCGTCGAG | | |
| | | | AAGCAGCACGCCAAGGGCAAGCTGACGGCTCGTGAACG | | |
| | | | CATCGACCTCCTCCTCGACGAGGGTTCCTTCGTCGAGC | | |
| | | | TGGACGAGTTCGCCCGGCACCGCTCCACCAACTTCGGC | | |
| | | | CTCGACGCCAACCGCCCCTACGGCGACGGCGTCGTCAC | | |
| | | | CGGCTACGGCACCGTCGACGGCCGCCCCGTGGCCGTCT | | |
| | | | TCTCCCAGGACTTCACCGTCTTCGGCGGCGCGCTGGGC | | |
| | | | GAGGTCTACGGCCAGAAGATCGTCAAGGTGATGGACTT | | |
| | | | CGCCCTCAAGACCGGCTGCCCGGTCGTCGGCATCAACG | | |
| | | | ACTCCGGCGGCGCCCGCATCCAGGAGGGCGTGGCCTCC | | |
| | | | CTCGGCGCCTACGGCGAGATCTTCCGCCGCAACACCCA | | |
| | | | CGCCTCCGGCGTGATCCCGCAGATCAGCCTGGTCGTCG | | |
| | | | GCCCGTGTGCGGGCGGCGCGGTGTACTCCCCCGCGATC | | |
| | | | ACCGACTTCACGGTGATGGTGGACCAGACCAGCCACAT | | |
| | | | GTTCATCACCGGTCCCGACGTCATCAAGACGGTCACCG | | |
| | | | GCGAGGACGTCGGCTTCGAGGAGCTGGGCGGCGCCCGC | | |
| | | | ACCCACAACTCCACCTCGGGCGTGGCCCACCACATGGC | | |
| | | | CGGCGACGAGAAGGACGCGGTCGAGTACGTCAAGCAGC | | |
| | | | TCCTGTCGTACCTGCCGTCCAACAACCTCTCCGAGCCC | | |
| | | | CCCGCCTTCCCGGAGGAGGCGGACCTCGCGGTCACGGA | | |
| | | | CGAGGACGCCGAGCTGGACACGATCGTCCCGGACTCGG | | |
| | | | CGAACCAGCCCTACGACATGCACTCCGTCATCGAGCAC | | |
| | | | GTCCTGGACGACGCCGAGTTCTTCGAGACGCAACCCCT | | |
| | | | CTTCGCGCCGAACATCCTCACCGGCTTCGGCCGCGTGG | | |
| | | | AGGGCCGCCCGGTCGGCATCGTCGCCAACCAGCCCATG | | |
| | | | CAGTTCGCCGGCTGCCTGGACATCACGGCCTCCGAGAA | | |
| | | | GGCGGCCCGCTTCGTGCGCACCTGCGACGCCTTCAACG | | |
| | | | TCCCCGTCCTCACCTTCGTGGACGTCCCCGGCTTCCTG | | |
| | | | CCCGGCGTCGACCAGGAGCACGACGGCATCATCCGCCG | | |
| | | | CGGCGCCAAGCTGATCTTCGCCTACGCCGAGGCCACGG | | |
| | | | TGCCGCTCATCACGGTCATCACCCGCAAGGCCTTCGGC | | |
| | | | GGCGCCTACGACGTCATGGGCTCCAAGCACCTGGGCGC | | |
| | | | CGACCTCAACCTGGCCTGGCCCACCGCCCAGATCGCCG | | |
| | | | TCATGGGCGCCCAAGGCGCGGTCAACATCCTGCACCGC | | |
| | | | CGCACCATCGCCGACGCCGGTGACGACGCCGAGGCCAC | | |
| | | | CCGGGCCCGCCTGATCCAGGAGTACGAGGACGCCCTCC | | |
| | | | TCAACCCCTACACGGCGGCCGAACGCGGCTACGTCGAC | | |
| | | | GCCGTGATCATGCCCTCCGACACTCGCCGCCACATCGT | | |
| | | | CCGCGGCCTGCGCCAGCTGCGCACCAAGCGCGAGTCCC | | |
| | | | TGCCCCCGAAGAAGCACGGCAACATCCCCCTGTAA | | |
| dtsR1 | Mycobacterium | Z92771.1 | ATGACAAGCGTTACCGACCGCTCGGCTCATTCCGCAGA | 139 | |
| | tuberculosis | | GCGGTCCACCGAGCACACCATCGACATCCACACCACCG | | |
| | (use this to | | CGGGCAAGCTGGCGGAGCTGCACAAACGCAGGGAAGAG | | |
| | clone M. | | TCGCTGCACCCCGTCGGTGAGGATGCCGTCGAAAAAGT | | |
| | smegmatis | | ACACGCCAAGGGCAAGCTGACGGCTCGCGAGCGTATCT | | |
| | gene) | | ACGCGTTGCTGGATGAGGATTCGTTCGTCGAGCTGGAC | | |
| | | | GCGCTGGCCAAACACCGCAGCACCAACTTCAATCTCGG | | |
| | | | TGAAAAACGCCCGCTCGGCGACGGCGTGGTCACCGGCT | | |
| | | | ACGGCACCATCGACGGGCGCGACGTGTGCATCTTCAGC | | |
| | | | CAGGACGCCACGGTGTTTGGCGGCAGCCTTGGCGAGGT | | |
| | | | GTACGGCGAGAAAATCGTCAAGGTCCAGGAACTGGCGA | | |
| | | | TCAAGACCGGCCGTCCGCTCATCGGCATCAACGACGGT | | |
| | | | GCTGGCGCGCGCATCCAGGAAGGTGTCGTCTCGCTGGG | | |
| | | | CCTGTACAGCCGTATCTTTCGCAACAACATCCTGGCCT | | |
| | | | CCGGCGTCATCCCGCAAATCTCGTTGATCATGGGAGCC | | |
| | | | GCCGCCGGTGGGCACGTCTACTCCCCCGCCCTGACCGA | | |
| | | | CTTCGTGATCATGGTCGATCAGACCAGCCAGATGTTCA | | |
| | | | TCACCGGGCCCGACGTCATCAAGACCGTCACCGGCGAG | | |
| | | | GAAGTCACCATGGAAGAACTCGGCGGCGCCCACACCCA | | |
| | | | CATGGCCAAGTCGGGTACGGCACACTACGCCGCATCGG | | |
| | | | GCGAACAGGACGCCTTCGACTACGTTCGCGAGCTGCTG | | |
| | | | AGCTACCTGCCGCCCAACAACTCCACCGACGCGCCCCG | | |
| | | | ATACCAAGCCGCAGCCCCGACAGGGCCCATCGAGGAGA | | |
| | | | ACCTCACCGACGAGGACCTCGAATTGGATACGCTGATC | | |
| | | | CCGGACTCGCCCAACCAGCCCTATGACATGCACGAGGT | | |
| | | | GATCACCCGGCTCCTCGACGACGAATTCCTGGAGATAC | | |
| | | | AGGCCGGTTACGCCCAAAACATCGTGGTGGGGTTCGGG | | |
| | | | CGCATCGACGGCCGGCCAGTCGGCATTGTCGCCAACCA | | |
| | | | GCCGACACACTTCGCCGGCTGCCTGGATATCAACGCCT | | |
| | | | CGGAGAAAGCGGCCCGGTTTGTGCGGACCTGCGACTGC | | |
| | | | TTCAATATCCCCATCGTCATGCTGGTGGACGTCCCGGG | | |
| | | | CTTCCTGCCGGGCACCGACCAGGAATACAACGGCATCA | | |
| | | | TCCGGCGCGGCGCCAAGCTGCTCTACGCCTACGGCGAG | | |
| | | | GCCACCGTGCCAAAGATCACGGTCATCACCCGCAAGGC | | |
| | | | CTACGGCGGTGCGTACTGCGTTATGGGCTCCAAAGACA | | |
| | | | TGGGCTGCGACGTCAACCTGGCGTGGCCGACCGCGCAG | | |
| | | | ATCGCGGTGATGGGCGCCTCCGGCGCAGTGGGCTTCGT | | |
| | | | GTACCGCCAGCAGCTGGCCGAGGCCGCCGCCAACGGCG | | |
| | | | AGGACATCGACAAGCTGCGGCTGCGGCTCCAGCAGGAG | | |
| | | | TACGAGGACACACTGGTCAACCCGTACGTGGCCGCCGA | | |
| | | | ACGCGGATACGTCGACGCGGTGATCCCGCCGTCGCATA | | |
| | | | CTCGCGGCTACATCGGGACCGCGCTGCGGCTGCTGGAA | | |
| | | | CGCAAGATCGCGCAGCTGCCGCCCAAAAAGCATGGGAA | | |
| | | | CGTGCCCCTGTGA | | |
| dtsR1 | Mycobacterium | U00012.1 | ATGACAAGCGTTACCGACCACTCGGCTCATTCAATGGA | 140 | |
| | leprae (use this | | ACGCGCTGCCGAGCACACGATCAATATCCACACCACGG | | |
| | to clone M. | | CAGGCAAGCTGGCCGAGCTGCATAAGCGGACCGAAGAA | | |
| | smegmatis | | GCGCTGCATCCGGTCGGTGCAGCTGCCTTCGAGAAGGT | | |
| | gene) | | ACACGCTAAGGGTAAGTTTACCGCCCGCGAGCGCATCT | | |
| | | | ACGCCCTATTGGACGACGACTCATTCGTCGAACTCGAC | | |
| | | | GCACTGGCCAGACACCGCAGCACCAACTTCGGCCTCGG | | |
| | | | TGAAAACCGCCCGGTAGGCGATGGCGTGGTCACCGGCT | | |
| | | | ACGGCACCATCGACGGCCGCGACGTATGCATCTTCAGC | | |
| | | | CAGGACGTCACGGTGTTCGGCGGCAGCCTGGGCGAAGT | | |
| | | | GTATGGCGAGAAGATCGTCAAGGTCCAGGAACTGGCGA | | |
| | | | TCAAGACCGGCCGTCCGCTTATCGGCATCAACGACGGC | | |
| | | | GCGGGCGCGCGTATCCAAGAAGGCGTCGTCTCGCTCGG | | |
| | | | CCTGTACAGCCGGATTTTCCGCAACAATATCTTGGCCT | | |
| | | | CCGGCGTCATCCCGCAGATCTCGCTGATCATGGGAGCG | | |
| | | | GCCGCCGGTGGACACGTGTATTCCCCAGCACTGACCGA | | |
| | | | CTTCGTGGTTATGGTCGACCAAACCAGCCAGATGTTCA | | |
| | | | TCACCGGACCCGACGTCATCAAGACCGTCACCGGCGAG | | |
| | | | GACGTCACCATGGAGGAGCTGGGTGGCGCCCATACCCA | | |
| | | | CATGGCCAAGTCGGGTACCGCACACTATGTAGCATCGG | | |
| | | | GCGAGCAAGACGCCTTCGATTGGGTGCGCGATGTGTTG | | |
| | | | AGCTACCTGCCGTCAAACAACTTCACCGACGCGCCGCG | | |
| | | | GTATTCTAAGCCCGTTCCTCACGGCTCCATTGAAGACA | | |
| | | | ACCTGACCGCTAAAGACTTGGAGTTGGACACGCTTATC | | |
| | | | CCGGACTCGCCGAACCAACCGTACGACATGCACGAAGT | | |
| | | | GGTGACCCGCCTCCTCGACGAGGAAGAGTTCCTTGAGG | | |
| | | | TGCAAGCCGGTTACGCCACCAACATCGTCGTCGGGCTC | | |
| | | | GGACGCATAGATGACCGACCGGTGGGCATCGTTGCCAA | | |
| | | | CCAACCCATCCAGTTCGCCGGCTGTCTAGACATCAACG | | |
| | | | CCTCGGAAAAGGCAGCCCGATTTGTGCGGGTCTGCGAC | | |
| | | | TGCTTCAACATCCCGATCGTGATGTTGGTGGATGTTCC | | |
| | | | AGGCTTCCTGCCTGGCACCGAGCAAGAATATGATGGCA | | |
| | | | TCATCCGACGCGGCGCAAAGCTGCTCTTCGCCTACGGC | | |
| | | | GAAGCCACCGTACCCAAGATCACCGTCATCACCCGCAA | | |
| | | | GGCCTACGGTGGCGCTTACTGCGTGATGGGCTCCAAAA | | |
| | | | ATATGGGCTGCGACGTCAACCTGGCTTGGCCGACCGCA | | |
| | | | CAGATTGCGGTGATGGGTGCCTCCGGCGCAGTAGGCTT | | |
| | | | CGTGTACCGCAAGGAACTGGCCCAAGCGGCCAAGAACG | | |
| | | | GCGCCAATGTTGATGAGCTACGCCTGCAGCTGCAGCAA | | |
| | | | GAGTACGAGGACACCCTGGTGAACCCGTACATCGCCGC | | |
| | | | CGAACGAGGTTACGTCGATGCGGTGATCCCGCCGTCAC | | |
| | | | ACACTCGCGGCTACATTGCCACGGCGCTTCACCTGTTG | | |
| | | | GAGCGCAAGATCGCACACCTTCCCCCCAAGAAGCACGG | | |
| | | | GAACATTCCGCTGTGA | | |
| dtsR1 | Corynebacterium | NC_003450 | ATGACCATTTCCTCACCTTTGATTGACGTCGCCAACCT | 259 | |
| | glutamicum | | TCCAGACATCAACACCACTGCCGGCAAGATCGCCGACC | | |
| | | | TTAAGGCTCGCCGCGCGGAAGCCCATTTCCCCATGGGT | | |
| | | | GAAAAGGCAGTAGAGAAGGTCCACGCTGCTGGACGCCT | | |
| | | | CACTGCCCGTGAGCGCTTGGATTACTTACTCGATGAGG | | |
| | | | GCTCCTTCATCGAGACCGATCAGCTGGCTCGCCACCGC | | |
| | | | ACCACCGCTTTCGGCCTGGGCGCTAAGCGTCCTGCAAC | | |
| | | | CGACGGCATCGTGACCGGCTGGGGCACCATTGATGGAC | | |
| | | | GCGAAGTCTGCATCTTCTCGCAGGACGGCACCGTATTC | | |
| | | | GGTGGCGCGCTTGGTGAGGTGTACGGCGAAAAGATGAT | | |
| | | | CAAGATCATGGAGCTGGCAATCGACACCGGCCGCCCAT | | |
| | | | TGATCGGTCTTTACGAAGGCGCTGGCGCTCGTATTCAG | | |
| | | | GACGGCGCTGTCTCCCTGGACTTCATTTCCCAGACCTT | | |
| | | | CTACCAAAACATTCAGGCTTCTGGCGTTATCCCACAGA | | |
| | | | TCTCCGTCATCATGGGCGCATGTGCAGGTGGCAACGCT | | |
| | | | TACGGCCCAGCTCTGACCGACTTCGTGGTCATGGTGGA | | |
| | | | CAAGACCTCCAAGATGTTCGTTACCGGCCCAGACGTGA | | |
| | | | TCAAGACCGTCACCGGCGAGGAAATCACCCAGGAAGAG | | |
| | | | CTTGGCGGAGCAACCACCCACATGGTGACCGCTGGTAA | | |
| | | | CTCCCACTACACCGCTGCGACCGATGAGGAAGCACTGG | | |
| | | | ATTGGGTACAGGACCTGGTGTCCTTCCTCCCATCCAAC | | |
| | | | AATCGCTCCTACGCACCGATGGAAGACTTCGACGAGGA | | |
| | | | AGAAGGCGGCGTTGAAGAAAACATCACCGCTGACGATC | | |
| | | | TGAAGCTCGACGAGATCATCCCAGATTCCGCGACCGTT | | |
| | | | CCTTACGACGTCCGCGATGTCATCGAATGCCTCACCGA | | |
| | | | CGATGGCGAATACCTGGAAATCCAGGCAGACCGCGCAG | | |
| | | | AAAACGTTGTTATTGCATTCGGCCGCATCGAAGGCCAG | | |
| | | | TCCGTTGGCTTTGTTGCCAACCAGCCAACCCAGTTCGC | | |
| | | | TGGCTGCCTGGACATCGACTCCTCTGAGAAGGCAGCTC | | |
| | | | GCTTCGTCCGCACCTGCGACGCGTTCAACATCCCAATC | | |
| | | | GTCATGCTTGTCGACGTCCCCGGCTTCCTCCCAGGCGC | | |
| | | | AGGCCAGGAGTACGGTGGCATTCTGCGTCGTGGCGCAA | | |
| | | | AGCTGCTCTACGCATACGGCGAAGCAACCGTTCCAAAG | | |
| | | | ATCACCGTCACCATGCGTAAGGCTTACGGCGGAGCGTA | | |
| | | | CTGCGTGATGGGTTCCAAGGGCTTGGGCTCTGACATCA | | |
| | | | ACCTTGCATGGCCAACCGCACAGATCGCCGTCATGGGC | | |
| | | | GCTGCTGGCGCAGTTGGATTCATCTACCGCAAGGAGCT | | |
| | | | CATGGCAGCTGATGCCAAGGGCCTCGATACCGTAGCTC | | |
| | | | TGGCTAAGTCCTTCGAGCGCGAGTATGAAGACCACATG | | |
| | | | CTCAACCCGTACCACGCTGCAGAACGTGGCCTGATCGA | | |
| | | | CGCCGTGATCCTGCCAAGCGAAACCCGCGGACAGATTT | | |
| | | | CCCGCAACCTTCGCCTGCTCAAGCACAAGAACGTCACT | | |
| | | | CGCCCTGCTCGCAAGCACGGCAACATGCCACTG | | |
| metH | Thermobifida | NZ_AAAQ010 | ATGAGCGCTCGACTCTCCTTCCGTGAAGTCCTCGGTTC | 141 | |
| | fusca | 00042.1 | CCGCGTCCTCGTCGCCGACGGGGCGATGGGAACGATGC | | |
| | | | TTCAGACATACGACCTGAGCATGGACGACTTCGAGGGA | | |
| | | | CACGAGGGGTGTAACGAGGTCCTCAACATCACCCGGCC | | |
| | | | CGACGTGGTCCGGGAGATCCACGAGGCCTACCTGCAGG | | |
| | | | CCGGCGTCGACTGTGTCGAAACCAACACGTTCGGCGCG | | |
| | | | AACTTCGGAAACCTCGGCGAATACGGCATCGCGGAACG | | |
| | | | CACCTACGAACTGGCTGAAGCCGGTGCCCGCCTGGCCC | | |
| | | | GCGAAGCCGCCGACGCGTACACCACTGCCGATCACGTC | | |
| | | | CGCTACGTCCTCGGCTCTGTGGGGCCCGGGACGAAGCT | | |
| | | | GCCCACCCTTGGCCACGCCCCGTACGCTGTGCTGCGCG | | |
| | | | ACCACTACGAACAGTGCGCACGCGGGCTCATTGACGGC | | |
| | | | GGTGTCGACGCGATCGTGATCGAAACCTGCCAGGACTT | | |
| | | | GCTGCAGGCGAAAGCCGCGATCGTGGGGGCACGGCGGG | | |
| | | | CCCGCAAGGCCGCGGGTACCGACACGCCGATCATCGTC | | |
| | | | CAGGTGACGATTGAAACCACGGGGACCATGCTGGTGGG | | |
| | | | CTCCGAGATCGGTGCGGCACTGACCTCGCTGGAACCGC | | |
| | | | CAGGGGTCGACATGATCGGCCTCAACTGCGCTACCGGT | | |
| | | | CCAGCAGAGATGAGCGAGCACCTGCGCTACCTCTCCCA | | |
| | | | CCACTCCCGCATCCCCCTCTCCTGCATGCCGAACGCGG | | |
| | | | GCCTGCCTGAGCTGGGGGCGGACGGGGCCGTCTACCCG | | |
| | | | CTGCAGCCGCATGAGCTCACCGAAGCACACGACACGTT | | |
| | | | CATCCGCGAGTTTGGCCTGGCCCTGGTGGGCGGCTGCT | | |
| | | | GCGGCACCACCCCTGAGCACCTCGCCCAAGTGGTGGAG | | |
| | | | CGGGTGCAGGGACGCGGCGTGCCGGACCGCAAACCGCA | | |
| | | | CGTCGAACCCGCCGCCGCCTCTATCTACCAGAGCGTCC | | |
| | | | CGTTCCGCCAGGACACCAGCTACCTGGCGATCGGGGAA | | |
| | | | CGCACCAACGCCAACGGCTCCAAGGCGTTCCGCGAAGC | | |
| | | | CATGCTCGCGGAACGCTACGACGACTGTGTGGAGATCG | | |
| | | | CCCGCCAGCAGATCCGCGACGGCGCGCACATGCTCGAC | | |
| | | | CTGTGCGTCGACTATGTGGGACGCGACGGGGTGCGCGA | | |
| | | | TATGCGGGAGCTGGCTTCCCGGCTGGCCACCGCCTCCA | | |
| | | | CGCTGCCGCTCGTACTGGACTCCACCGAAGTAGCGGTA | | |
| | | | CTGGAAGCTGGACTGGAGATGCTGGGCGGGCGCGCCGT | | |
| | | | GCTCAACTCGGTCAACTACGAGGACGGCGACGGCCCTG | | |
| | | | ACTCCCGGTTCGCCAAGGTCGCCGCGCTGGCGGTGGAG | | |
| | | | CACGGGGCGGCCCTCATGGCGCTGACCATCGACGAGCA | | |
| | | | GGGGCAGGCGCGGACCGCGGAACGGAAAGTGGAGGTCG | | |
| | | | CCGAGCGGCTCATCCGGCAGCTCACCACCGAGTACGGC | | |
| | | | ATCCGCAAGCACGACATCATCGTGGACTGCCTGACCTT | | |
| | | | CACGATCGCAACCGGACAGGAGGAGTCGCGGCGCGACG | | |
| | | | CTCTGGAAACCATCGAGGCGATCCGTGAACTGAAGCGG | | |
| | | | CGCCACCCGGACGTGCAGACCACGCTGGGCGTGTCCAA | | |
| | | | CGTCTCCTTCGGGCTCAACCCGGCTGCCCGCATTGTGC | | |
| | | | TCAACTCGGTGTTCCTCCACGAGTGCGTCCAGGCCGGC | | |
| | | | TTGGACTCCGCGATCGTGCACGCCTCCAAGATCCTGCC | | |
| | | | GATCAACCGCATCCCCGAGGAGCAGCGGCAGGTGGCGT | | |
| | | | TGGACATGATCTACGACCGCCGCACCGATGACTACGAC | | |
| | | | CCGCTGCAACGCTTCCTGCAGCTTTTCGAAGGAGTGGA | | |
| | | | CGCGCAGGCGATGCGCGCCTCGCGCGAGGAAGAGCTGG | | |
| | | | CCGCGCTGCCGCTGTGGGAGCGCCTGGAGCGCCGTATC | | |
| | | | GTCGACGGGGAAGCCGCCGGCATGGAAGCGGACCTGGA | | |
| | | | CGAAGCGCTCACCCAGCGGTCCGCGCTGGACATCATCA | | |
| | | | ACACCACGCTGCTGGCGGGGATGAAGACCGTCGGCGAC | | |
| | | | CTGTTCGGCTCCGGGCAGATGCAGCTCCCGTTCGTGCT | | |
| | | | GAAGTCGGCCGAGGTGATGAAGGCCGCCGTGGCCTATC | | |
| | | | TGGAGCCGCACATGGAGAAGGTGGACGGCGACCTCGGC | | |
| | | | AAGGGGCGGATCGTGCTGGCCACGGTCAAGGGCGACGT | | |
| | | | CCACGACATCGGCAAGAACCTTGTGGACATCATCCTGT | | |
| | | | CCAACAACGGCTACGAGGTCATCAACCTGGGGATCAAG | | |
| | | | CAGCCCATCTCCGCGATTCTGGAGGCGGCCGAGCGGCA | | |
| | | | CCGCGCCGACGTGATCGGCATGTCCGGCCTGCTGGTGA | | |
| | | | AGTCCACGGTGGTGATGCGGGAGAACCTGGAGGAGATG | | |
| | | | AACGCCCGCGGGGTCGCTGACCGCTACCCGGTCCTGCT | | |
| | | | GGGCGGTGCCGCGTTGACCCGCTCCTATGTGGAACAGG | | |
| | | | ACCTCGCCGAGATTTTCAAAGGCGAGGTGCGCTATGCC | | |
| | | | CGCGACGCTTTTGAAGGCTTGAAGCTCATGGACGCCAT | | |
| | | | CATGGCGGTCAAACGCGGGGTGAAGGGGGCTAAGCTGC | | |
| | | | CGCCGCTGCGCACCCGCCGGGTGAAGCGGGGCGCACAG | | |
| | | | CTTACCGTCACCGAGCCGGAGAAGATGCCGACGCGCAG | | |
| | | | CGACGTGGCCACCGACAACCCGGTGCCGACCCCGCCGT | | |
| | | | TCTGGGGGGACCGCATCTGCAAGGGGATTCCGCTCGCC | | |
| | | | GACTACGCGGCTTTCCTGGATGAGCGCGCCACGTTCAT | | |
| | | | GGGCCAGTGGGGGCTGCGCGGCTCCCGCGGCGACGGCC | | |
| | | | CCACCTACGAGGAGCTGGTGGAGACGGAGGGGCGGCCG | | |
| | | | CGGCTGCGCATGTGGCTGGACCGGATCCAGACCGAGGG | | |
| | | | GTGGCTGGAGCCGGCGGTCGTCTACGGCTACTACCGCT | | |
| | | | GCTACAGCGAAGGCAACGACCTGGTCGTCCTCGGTGAG | | |
| | | | GACGAAAACGAGCTGACCCGGTTCACGTTCCCGCGGCA | | |
| | | | GCGCCGCGACCGGCACCTGTGCCTGGCTGACTTCTTCC | | |
| | | | GCCCCAAGGAGTCCGGGGAACTGGACACGGTGGCGTTC | | |
| | | | CAGGTCGTCACCGTCGGTTCGACGATCAGCAAGGCGAC | | |
| | | | CGCGGAGCTGTTCGAGAAGAACGCGTACCGGGACTACT | | |
| | | | TGGAGCTCCACGGGCTGTCCGTGCAGTTGACGGAGGCA | | |
| | | | CTCGCGGAGTACTGGCACACCCGGGTCCGCGCCGAGCT | | |
| | | | GGGCTTCGCCGGGGAGGATCCCGACCCGGCCGATTTGG | | |
| | | | ACGCCTACTTTAAGCTCGGCTATCGAGGCGCCCGTTTC | | |
| | | | TCCCTGGGGTACGGGGCCTGCCCCAACTTGGAGGACCG | | |
| | | | CGCCAAGATCGTGGCCCTGCTGCGTCCGGAACGGGTTG | | |
| | | | GGGTGACGTTGTCCGAGGAGTTCCAGCTTGTTCCCGAA | | |
| | | | CAGTCCACTGACGCGATCGTTGTCCATCACCCCGAGGC | | |
| | | | GAAATACTTCAACGTATGA | | |
| metH | Streptomyces | AL939109.1 | ATGGCCTCGTCGCCATCCACCCCGCCCGCCGACACCCG | 142 | |
| | coelicolor | | CACCCGCGTGTCCGCCCTCCGAGAGGCCCTCGCCACCC | | |
| | | | GCGTGGTGGTCGCCGACGGCGCCATGGGCACCATGCTC | | |
| | | | CAGGCCCAGAACCCCACGCTGGACGACTTCCAGCAGCT | | |
| | | | CGAAGGGTGCAACGAGGTCCTGAACCTCACCCGGCCCG | | |
| | | | ACATCGTCCGCTCGGTGCACGAGGAGTACTTCGCGGCC | | |
| | | | GGCGTCGACTGCGTCGAGACCAACACCTTCGGCGCCAA | | |
| | | | CCACTCCGCCCTGGGCGAGTACGACATCCCCGAGCGCG | | |
| | | | TCCACGAACTGTCCGAGGCCGGCGCCCGCGTCGCCCGC | | |
| | | | GAGGTCGCCGACGAGTTCGGCGCCCGCGACGGCCGGCA | | |
| | | | GCGCTGGGTGCTGGGCTCCATGGGCCCCGGCACCAAGC | | |
| | | | TCCCCACCCTCGGCCACGCCCCGTACACCGTCCTGCGC | | |
| | | | GACGCCTACCAGCGCAACGCCGAGGGACTGGTCGCGGG | | |
| | | | CGGCGCGGACGCACTGCTGGTGGAGACCACGCAGGACC | | |
| | | | TGCTCCAGACCAAGGCCTCGGTGCTCGGCGCCCGGCGC | | |
| | | | GCCCTGGACGTCCTCGGCCTCGACCTGCCGCTCATCGT | | |
| | | | GTCCGTCACCGTCGAGACCACCGGCACCATGCTGCTCG | | |
| | | | GCTCGGAGATCGGCGCCGCGCTCACCGCGCTGGAACCG | | |
| | | | CTCGGCATCGACATGATCGGCCTGAACTGCGCCACCGG | | |
| | | | CCCCGCCGAGATGAGCGAGCACCTGCGCTACCTCGCCC | | |
| | | | GGCACTCCCGCATCCCGCTGACCTGCATGCCCAACGCC | | |
| | | | GGTCTGCCCGTCCTCGGCAAGGACGGCGCCCACTACCC | | |
| | | | GCTGACCGCGCCCGAGCTGGCCGACGCACACGAGACCT | | |
| | | | TCGTGCGCGAGTACGGCCTGTCCCTGGTCGGCGGCTGC | | |
| | | | TGCGGCACCACGCCCGAGCACCTGCGCCAGGTCGTCGA | | |
| | | | GCGGGTCCGGGACACCGCCCCCACCGCACGCGACCCGC | | |
| | | | GCCCCGAGCCCGGCGCCGCCTCGCTCTACCAGACCGTG | | |
| | | | CCCTTCCGCCAGGACACCTCCTACCTGGCCATCGGCGA | | |
| | | | GCGCACCAACGCCAACGGGTCCAAGAAGTTCCGCGAGG | | |
| | | | CCATGCTGGACGGCCGCTGGGACGACTGCGTCGAGATG | | |
| | | | GCCCGCGACCAGATCCGCGAAGGCGCGCACATGCTCGA | | |
| | | | CCTCTGCGTCGACTACGTCGGCCGGGACGGCGTCGCCG | | |
| | | | ACATGGAGGAACTGGCCGGCCGGTTCGCCACCGCCTCC | | |
| | | | ACGCTGCCGATCGTCCTCGACTCCACCGAGGTCGACGT | | |
| | | | CATCCGGGCCGGCCTGGAGAAGCTCGGCGGCCGCGCGG | | |
| | | | TGATCAACTCGGTCAACTACGAGGACGGCGCCGGCCCC | | |
| | | | GAGTCCCGGTTCGCCCGCGTCACGAAGCTCGCCCGGGA | | |
| | | | GCACGGCGCCGCGCTGATCGCGCTGACCATCGACGAGG | | |
| | | | TGGGACAGGCCCGCACCGCCGAGAAGAAGGTCGAGATC | | |
| | | | GCCGAACGGCTCATCGACGACCTCACCGGCAACTGGGG | | |
| | | | CATCCACGAGTCCGACATCCTCGTCGACTGCCTGACCT | | |
| | | | TCACCATCTGCACCGGCCAGGAGGAGTCCCGCAAGGAC | | |
| | | | GGCCTGGCCACCATCGAGGGCATCCGGGAACTCAAGCG | | |
| | | | GCGCCACCCGGACGTGCAGACCACGCTCGGCCTGTCGA | | |
| | | | ACATCTCCTTCGGCCTCAACCCGGCCGCCCGCATCCTG | | |
| | | | CTCAACTCCGTCTTCCTCGACGAATGCGTCAAGGCCGG | | |
| | | | CCTGGACTCGGCCATCGTGCACGCGAGCAAGATCCTGC | | |
| | | | CGATCGCCCGCTTCGACGAGGAGCAGGTCACCACCGCC | | |
| | | | CTCGACTTGATCTACGACCGCCGCCGCGAGGGCTACGA | | |
| | | | CCCCCTGCAAAAGCTCATGCAGCTCTTCGAGGGCGCCA | | |
| | | | CCGCCAAGTCGCTGAAGGCCTCCAAGGCCGAGGAACTG | | |
| | | | GCCGCCCTCCCGCTGGAGGAGCGCCTCAAGCGCCGCAT | | |
| | | | CATCGACGGCGAGAAGAACGGCCTCGAACAGGACCTCG | | |
| | | | ACGAGGCCCTCCGGGAGCGCCCGGCCCTCGAGATCGTC | | |
| | | | AACGACACCCTGCTCGACGGTATGAAGGTCGTCGGCGA | | |
| | | | GCTGTTCGGCTCCGGCCAGATGCAGCTGCCGTTCGTGC | | |
| | | | TCCAGTCCGCCGAGGTCATGAAGACCGCGGTGGCCCAC | | |
| | | | CTGGAGCCGCACATGGAGAAGACCGACGACGACGGCAA | | |
| | | | GGGCACGATCGTGCTGGCCACCGTCCGCGGCGACGTCC | | |
| | | | ACGACATCGGCAAGAACCTCGTCGACATCATCCTGTCC | | |
| | | | AACAACGGCTACAACGTCGTCAACCTCGGCATCAAGCA | | |
| | | | GCCCGTCTCCGCGATCCTGGAAGCGGCCGACGAGCACC | | |
| | | | GGGCCGACGTCATCGGCATGTCCGGCCTCCTCGTCAAG | | |
| | | | TCCACGGTGATCATGAAGGAGAACCTGGAGGAGCTGAA | | |
| | | | CCAGCGCAAGCTGGCCGCCGACTACCCGGTCATCCTCG | | |
| | | | GCGGCGCCGCCCTCACCAGGGCCTACGTCGAACAGGAC | | |
| | | | CTGCACGAGATCTACGACGGCGAGGTCCGCTACGCCCG | | |
| | | | CGACGCCTTCGAGGGCCTGCGCCTCATGGACGCCCTCA | | |
| | | | TCGGCATCAAGCGCGGCGTGCCCGGCGCCAAGCTGCCG | | |
| | | | GAGCTGAAGCAGCGCCGGGTGCGGGCCGCCACCGTCGA | | |
| | | | GATCGACGAGCGCCCCGAGGAAGGCCACGTCCGCTCCG | | |
| | | | ACGTCGCCACCGACPACCCGGTCCCGACCCCGCCCTTC | | |
| | | | CGCGGCACCCGCGTCGTCAAGGGCATCCAGCTCAAGGA | | |
| | | | GTACGCCTCCTGGCTCGACGAGGGCGCCCTCTTCAAGG | | |
| | | | GCCAGTGGGGCCTCAAGCAGGCCCGCACCGGCGAGGGA | | |
| | | | CCCTCCTACGAGGAACTGGTCGAGTCCGAGGGCCGGCC | | |
| | | | GCGGCTGCGCGGCCTGCTCGACCGGCTCCAGACGGACA | | |
| | | | ACCTTTTGGAGGCGGCCGTGGTCTACGGCTACTTCCCC | | |
| | | | TGCGTCTCCAAGGACGACGACCTGATCGTCCTCGACGA | | |
| | | | CGACGGCAACGAACGCACCCGCTTCACCTTCCCCCGCC | | |
| | | | AGCGCCGCGGCCGGCGCCTGTGCCTGGCCGACTTCTTC | | |
| | | | CGCCCGGAGGAGTCCGGCGAGACCGACGTGGTCGGCTT | | |
| | | | CCAGGTCGTCACCGTCGGCTCCCGCATCGGCGAGGAGA | | |
| | | | CGGCCCGCATGTTCGAGGCCAACGCCTACCGCGACTAT | | |
| | | | CTCGAGCTGCACGGCCTGTCCGTGCAGCTCGCCGAGGC | | |
| | | | CCTCGCCGAGTACTGGCACGCGCGCGTGCGCTCGGAAC | | |
| | | | TCGGCTTCGCCGGGGAGGACCCGGCCGAGATGGAGGAC | | |
| | | | ATGTTCGCCCTGAAGTACCGGGGTGCCCGCTTCTCCCT | | |
| | | | CGGCTACGGCGCCTGCCCCGACCTGGAGGACCGCGCCA | | |
| | | | AGATCGCCGCCCTGCTGGAGCCCGAGCGCATCGGCGTC | | |
| | | | CACCTATCCGAGGAGTTCCAGCTCCACCCCGAGCAGTC | | |
| | | | CACCGACGCCATCGTCATCCACCACCCGGAGGCCAAGT | | |
| | | | ACTTCAACGCCCGCTGA | | |
| metH | Mycobacterium | Z97559.1 | GTGACTGCGGCCGACAAGCACCTCTACGACACCGATCT | 143 | |
| | tuberculosis | | GCTCGACGTCTTGTCGCAGCGAGTGATGGTCGGCGACG | | |
| | (use this to | | GTGCAATGGGAACCCAACTACAGGCCGCGGACCTCACG | | |
| | clone M. | | CTCGACGACTTCCGCGGCCTGGAGGGCTGCAACGAGAT | | |
| | smegmatis | | CCTCAACGAAACCCGCCCTGACGTGCTGGAAACCATTC | | |
| | gene) | | ACCGCAACTATTTCGAAGCGGGCGCCGACGCCGTCGAG | | |
| | | | ACGAACACGTTTGGCTGCAACCTGTCCAACCTCGGCGA | | |
| | | | CTACGACATCGCCGACAGGATCCGCGATCTATCACAGA | | |
| | | | AGGGCACCGCGATCGCACGCCGGGTGGCCGACGAGCTG | | |
| | | | GGCAGTCCCGACCGCAAGCGCTACGTGCTGGGGTCGAT | | |
| | | | GGGGCCGGGCACCAAGCTGCCGACTCTGGGCCACACCG | | |
| | | | AATACGCGGTGATCCGCGACGCCTACACCGAGGCCGCG | | |
| | | | CTGGGCATGCTGGACGGCGGAGCCGACGCCATCCTGGT | | |
| | | | GGAAACCTGCCAGGACCTACTGCAGCTGAAGGCGGCGG | | |
| | | | TGTTGGGGTCGCGGCGGGCGATGACGCGGGCCGGGCGG | | |
| | | | CACATTCCGGTGTTTGCCCACGTCACCGTCGAGACCAC | | |
| | | | CGGCACCATGCTGCTGGGCAGCGAGATCGGGGCGGCGT | | |
| | | | TGACCGCTGTCGAGCCGCTCGGTGTGGACATGATCGGC | | |
| | | | TTGAACTGCGCGACGGGTCCGGCCGAGATGAGCGAGCA | | |
| | | | CCTGCGCCACCTGTCCCGGCACGCCCGCATCCCGGTGT | | |
| | | | CGGTGATGCCCAACGCCGGGTTGCCGGTGCTGGGCGCC | | |
| | | | AAGGGCGCCGAATATCCGTTGCTGCCCGACGAATTGGC | | |
| | | | CGAGGCGCTGGCCGGCTTCATCGCCGAGTTCGGGCTCT | | |
| | | | CGCTGGTCGGTGGCTGCTGCGGCACCACCCCGGCCCAT | | |
| | | | ATCCGCGAAGTGGCTGCCGCGGTTGCGAACATCAAGCG | | |
| | | | TCCCGAGCGACAGGTCAGCTACGAGCCGTCGGTGTCGT | | |
| | | | CGCTGTACACCGCAATCCCGTTCGCCCAGGACGCCTCG | | |
| | | | GTTCTGGTGATCGGGGAGCGAACGAACGCCAACGGCTC | | |
| | | | CAAGGGTTTTCGTGAGGCGATGATCGCCGAGGACTACC | | |
| | | | AGAAGTGCCTGGACATCGCCAAGGACCAGACCCGCGAC | | |
| | | | GGCGCCCACCTGCTGGACCTGTGTGTGGACTACGTGGG | | |
| | | | CCGCGACGGTGTGGCCGACATGAAGGCGCTGGCCAGCC | | |
| | | | GGCTGGCCACGTCCTCGACGCTGCCGATCATGCTGGAC | | |
| | | | TCCACCGAAACCGCGGTGCTGCAGGCGGGTTTGGAGCA | | |
| | | | TCTGGGTGGCCGTTGCGCGATCAACTCGGTGAACTACG | | |
| | | | AGGACGGCGACGGCCCGGAATCGCGCTTTGCCAAGACC | | |
| | | | ATGGCGCTGGTCGCCGAGCACGGCGCGGCGGTGGTCGC | | |
| | | | GCTGACCATCGACGAAGAGGGCCAGGCCCGCACCGCGC | | |
| | | | AGAAGAAGGTCGAGATCGCCGAGCGGCTGATCAACGAC | | |
| | | | ATCACCGGCAACTGGGGCGTCGACGAATCATCCATCCT | | |
| | | | CATCGACACCTTGACGTTCACCATCGCCACCGGTCAGG | | |
| | | | AGGAGTCCCGCCGCGACGGCATCGAGACCATCGAGGCG | | |
| | | | ATCCGCGAACTGAAAAAGCGCCACCCGGATGTGCAGAC | | |
| | | | CACACTTGGTCTGTCCAACATCTCGTTTGGTCTCAATC | | |
| | | | CCGCAGCGCGCCAGGTGCTCAACTCGGTGTTCCTGCAC | | |
| | | | GAATGCCAAGAAGCGGGGCTGGATTCGGCGATCGTGCA | | |
| | | | CGCGTCGAAGATCCTGCCGATGAACCGGATTCCCGAGG | | |
| | | | AGCAACGCAACGTCGCCCTGGATCTGGTCTACGACCGC | | |
| | | | CGCCGCGAGGACTACGATCCGCTGCAGGAGCTGATGCG | | |
| | | | GCTGTTCGAAGGCGTGTCGGCGGCCTCCTCGAAAGAGG | | |
| | | | ACCGACTGGCTGAACTAGCTGGGCTGCCGCTGTTCGAA | | |
| | | | CGGCTGGCCCAACGCATCGTCGACGGCGAGCGCAACGG | | |
| | | | CCTGGACGCCGATCTCGACGAGGCGATGACGCAAAAGC | | |
| | | | CGCCGCTTCAGATCATCAACGAACATCTGCTGGCCGGC | | |
| | | | ATGAAGACGGTCGGCGAGCTCTTCGGCTCCGGCCAGAT | | |
| | | | GCAGCTGCCGTTCGTGCTGCAGTCGGCGGAGGTAATGA | | |
| | | | AAGCCGCCGTCGCGTATCTGGAACCGCACATGGAGCGC | | |
| | | | TCGGACGACGATTCGGGCAAGGGACGCATCGTGCTGGC | | |
| | | | CACCGTCAAGGGCGACGTGCACGACATCGGCAAGAACC | | |
| | | | TGGTCGACATCATCTTGAGCAACAACGGCTACGAAGTG | | |
| | | | GTCAACATCGGCATCAAGCAGCCAATCGCCACCATCCT | | |
| | | | CGAAGTCGCCGAGGACAAGAGCGCCGACGTGGTCGGCA | | |
| | | | TGTCGGGCCTGCTGGTGAAGTCGACCGTGGTGATGAAG | | |
| | | | GAAAACCTCGAGGAGATGAACACCCGGGGAGTCGCCGA | | |
| | | | AAAGTTCCCGGTGCTGCTCGGCGGCGCGGCGTTGACGC | | |
| | | | GCAGCTATGTCGAAAACGACCTGGCCGAGATCTACCAG | | |
| | | | GGCGAAGTGCATTACGCGCGAGACGCTTTCGAGGGCCT | | |
| | | | GAAGTTGATGGACACCATCATGAGCGCCAAGCGCGGCG | | |
| | | | AGGCGCCCGACGAAAACAGCCCGGAAGCCATTAAGGCG | | |
| | | | CGTGAGAAAGAAGCCGAACGTAAGGCCCGCCACCAGCG | | |
| | | | ATCCAAACGCATTGCCGCACAGCGCAAAGCCGCCGAAG | | |
| | | | AACCAGTCGAGGTGCCCGAACGCTCCGATGTCGCGGCC | | |
| | | | GACATCGAGGTCCCGGCGCCGCCGTTCTGGGGTTCGCG | | |
| | | | GATCGTCAAGGGCCTGGCGGTGGCCGACTACACCGGTC | | |
| | | | TGCTCGATGAGCGCGCATTGTTTTTGGGCCAGTGGGGT | | |
| | | | TTACGCGGCCAGCGCGGCGGTGAGGGTCCGTCCTACGA | | |
| | | | AGATCTCGTCGAGACCGAGGGCCGGCCGCGGCTGCGGT | | |
| | | | ACTGGTTGGACCGGCTGTCCACCGACGGCATCTTGGCG | | |
| | | | CACGCCGCCGTGGTGTACGGCTATTTCCCGGCGGTGTC | | |
| | | | CGAGGGCAACGACATCGTGGTGCTCACCGAGCCCAAGC | | |
| | | | CCGACGCCCCGGTGCGCTACCGGTTTCACTTCCCGCGC | | |
| | | | CAGCAGCGCGGTCGGTTTTTGTGCATTGCCGATTTCAT | | |
| | | | CCGCTCGCGGGAGCTGGCCGCCGAGCGTGGCGAGGTTG | | |
| | | | ACGTGCTGCCGTTCCAGCTGGTGACCATGGGTCAGCCG | | |
| | | | ATCGCGGATTTCGCCAACGAGCTGTTCGCGTCCAACGC | | |
| | | | CTACCGCGACTACCTGGAGGTGCACGGTATCGGCGTGC | | |
| | | | AGCTCACCGAGGCGCTGGCCGAGTACTGGCACCGGCGG | | |
| | | | ATCCGTGAGGAGCTCAAGTTCTCCGGGGATCGGGCGAT | | |
| | | | GGCGGCCGAGGATCCGGAGGCGAAAGAAGACTATTTCA | | |
| | | | AGCTCGGCTACCGCGGTGCTCGCTTTGCCTTCGGCTAC | | |
| | | | GGCGCATGCCCGGATCTGGAGGACCGCGCCAAGATGAT | | |
| | | | GGCGCTGCTGGAGCCCGAACGCATCGGTGTGACGTTAT | | |
| | | | CCGAGGAATTACAGCTGCATCCCGAACAGTCGACCGAC | | |
| | | | GCGTTCGTCCTGCACCATCCGGAAGCCAAGTACTTCAA | | |
| | | | CGTTTAA | | |
| metH | Mycobacterium | AL583921.1 | ATGCGTGTAACTGCCGCTAACCAACATCAGTACGACAC | 144 | |
| | leprae (use this | | CGATCTCCTCGAGACTTTGGCGCAGCGTGTGATGGTGG | | |
| | to clone M. | | GTGACGGCGCAATGGGTACTCAGCTCCAGGACGCGGAA | | |
| | smegmatis | | CTTACGTTAGATGATTTCCGCGGCCTGGAGGGCTGCAA | | |
| | gene) | | CGAGATTCTCAACGAAACGCGTCCTGACGTGCTGGAAA | | |
| | | | CCATCCACCGACGCTACTTCGAGGCAGGTGCGGACCTC | | |
| | | | GTCGAGACCAACACTTTCGGCTGCAACCTGTCCAACCT | | |
| | | | TGGTGACTACGACATCGCCGACAAGATCAGGGACTTGT | | |
| | | | CGCAGCGGGGCACCGTGATTGCGCGACGGGTCGCCGAC | | |
| | | | GAGCTGACCACCCCCGACCACAAGCGATACGTGCTGGG | | |
| | | | GTCGATGGGACCAGGCACCAAGTTGCCCACCCTGGGCC | | |
| | | | ACACCGAGTACCGGGTCGTTCGAGACGCCTACACCGAG | | |
| | | | TCGGCGTTAGGCATGCTGGACGGTGGCGCTGACGCCGT | | |
| | | | ACTGGTTGAAACCTGTCAGGACTTGCTGCAGCTCAAGG | | |
| | | | CTGCGGTGCTGGGCTCGCGGCGCGCGATGACACAGGCC | | |
| | | | GGTCGGCACATTCCGGTCTTCGTCCACGTGACTGTCGA | | |
| | | | GACGACCGGAACGATGCTGCTGGGAAGTGAGATCGGCG | | |
| | | | CTGCACTGGCTGCCGTCGAGCCGCTCGGTGTCGACATG | | |
| | | | ATCGGTTTGAACTGCGCAACGGGCCCCGCTGAGATGAG | | |
| | | | TGAGCATCTGCGGCACTTGTCCAAGCATGCCCGCATCC | | |
| | | | CGGTGTCGGTGATGCCCAACGCCGGGCTGCCGGTGCTG | | |
| | | | GGTGCCAAGGGAGCTGAATACCCGCTGCAGCCCGACGA | | |
| | | | ATTGGCCGAAGCTTTGGCTGGGTTCATCGCTGAATTTG | | |
| | | | GTCTTTCGTTGGTAGGTGGCTGCTGTGGTACCACCCCG | | |
| | | | GACCACATCCGGGAAGTGGCCGCAGCGGTAGCCAGATG | | |
| | | | CAACGACGGGACAGTGCCACGCGGTGAGCGTCATGTGA | | |
| | | | CCTATGAGCCGTCGGTATCGTCGCTGTATACAGCCATT | | |
| | | | CCATTCGCCCAAAAACCCTCGGTTCTGATGATCGGTGA | | |
| | | | GCGTACGAATGCCAACGGCTCCAAGGTTTTTCGTGAGG | | |
| | | | CAATGATCGCCGAGGACTATCAAAAGTGTCTAGATATC | | |
| | | | GCCAAGGACCAAACCCGTGGCGGCGCACACCTGCTGGA | | |
| | | | TCTGTGTGTCGATTACGTCGGCCGCAACGGTGTGGCCG | | |
| | | | ACATGAAGGCGTTGGCCGGTCGGCTTGCAACGGTGTCG | | |
| | | | ACATTGCCGATCATGCTGGACTCTACCGAAATACCGGT | | |
| | | | GCTGCAGGCAGGTTTGGAGCACCTGGGCGGGCGCTGCG | | |
| | | | TGATCAATTCCGTCAACTACGAGGACGGTGACGGTCCC | | |
| | | | GAGTCACGGTTTGTCAAGACCATGGAGCTGGTCGCCGA | | |
| | | | GCACGGAGCGGCGGTGGTTGCGCTGACCATCGACGAAC | | |
| | | | AGGGTCAGGCCCGCACCGTTGAGAAGAAGGTCGAAGTC | | |
| | | | GCGGAGCGGCTTATCAATGACATTACGAGTAACTGGGG | | |
| | | | CGTTGATAAATCGGCGATTCTCATCGATTGCTTGACTT | | |
| | | | TTACTATTGCCACTGGCCAGGAGGAGTCACGCAAAGAC | | |
| | | | GGCATTGAGACCATCGACGCGATTCGTGAGCTGAAGAA | | |
| | | | GCGGCACCCAGCGGTGCAGACTACGCTGGGGTTGTCCA | | |
| | | | ACATCTCCTTCGGTCTCAATCCTTCTGCACGCCAAGTT | | |
| | | | CTTAACTCTGTTTTTCTACATGAATGTCAGGAAGCAGG | | |
| | | | ACTGGATTCGGCGATTGTGCACGCTTCAAAGATATTGC | | |
| | | | CCATCAACCGGATACCCGAAGAACAGCGCCAGGCTGCG | | |
| | | | CTGGATCTAGTGTATGACCGCCGTCGCGAAGGCTACGA | | |
| | | | CCCATTGCAGAAGCTGATGTGGTTATTCAAAGGTGTGT | | |
| | | | CGTCGCCATCGTCGAAGGAAACACGGGAGGCAGAACTC | | |
| | | | GCTAAGCTGCCGTTGTTCGACCGGTTAGCACAGCGGAT | | |
| | | | CGTCGACGGCGAGCGCAACGGGTTAGATGTTGATCTCG | | |
| | | | ACGAGGCAATGACCCAGAAACCGCCGTTGGCGATCATC | | |
| | | | AACGAGAACCTGCTGGACGGCATGAAGACAGTCGGTGA | | |
| | | | ATTGTTCGGCTCTGGGCAGATGCAGCTGCCTTTCGTGT | | |
| | | | TGCAGTCGGCCGAGGTTATGAAAGCAGCGGTGGCTTAT | | |
| | | | CTAGAACCGCACATGGAGAAATCCGACTGTGACTTCGG | | |
| | | | TAAGGGGTTAGCCAAAGGACGGATTGTGCTGGCTACCG | | |
| | | | TCAAAGGAGATGTGCACGATATTGGCAAAAACCTCGTC | | |
| | | | GATATCATTCTGAGCAACAACGGCTACGAAGTGGTAAA | | |
| | | | CCTCGGCATCAAGCAGCCGATTACCAACATTCTCGAGG | | |
| | | | TGGCCGAGGACAAAAGCGCCGACGTAGTCGGGATGTCG | | |
| | | | GGCTTGCTGGTGAAATCGACTGTGATCATGAAGGAAAA | | |
| | | | CCTCGAGGAGATGAACACTCGCGGAGTCGCTGAGAAAT | | |
| | | | TCCCAGTGCTGCTCGGCGGCGCGGCGTTGACCCGCAGC | | |
| | | | TATGTGGAAAACGACCTGGCCGAAGTCTATGAGGGCGA | | |
| | | | AGTGCATTACGCACGAGACGCTTTCGAGGGTTTGAAGT | | |
| | | | TGATGGACACCATTATGAGCGCCAAGCGCGGCGAGGCG | | |
| | | | CTTGCGCCGGGGAGCCCGGAGTCCTTAGCTGCAGAAGC | | |
| | | | AGACCGCAATAAGGAAACTGAGCGCAAGGCACGTCATG | | |
| | | | AGCGGTCCAAACGCATTGCAGTGCAGCGTAAGGCTGCC | | |
| | | | GAAGAGCCAGTTGAGGTTCCCGAACGCTCCGATGTTCC | | |
| | | | GAGTGATGTCGAGGTTCCGGCGCCGCCGTTCTGGGGTT | | |
| | | | CGCGGATCATCAAGGGTCTGGCGGTGGCCGACTATACC | | |
| | | | GGGTTCCTCGACGAGCGCGCGTTGTTCTTGGGTCAGTG | | |
| | | | GGGATTACGTGGTGTGCGCGGCGGTGCGGGGCCCTCGT | | |
| | | | ACGAGGATTTGGTGCAGACCGAGGGCCGGCCGCGGTTG | | |
| | | | CGCTACTGGCTAGACCGATTGTCCACCTACGGCGTCTT | | |
| | | | GGCGTACGCCGCCGTGGTGTACGGTTACTTCCCGGCGG | | |
| | | | TGTCCGAAGACAACGATATTGTCGTGCTCGCTGAGCCG | | |
| | | | AGACCGGACGCCGAGCAGCGGTACCGGTTCACCTTCCC | | |
| | | | GCGTCAGCAACGCGGTCGGTTCCTGTGCATTGCCGATT | | |
| | | | TTATTCGATCCCGGGATCTGGCGACCGAGCGGAGTGAG | | |
| | | | GTGGATGTTTTGCCGTTCCAGCTGGTGACCATGGGTCA | | |
| | | | ACCCATTGCTGACTTCGTTGGCGAGTTGTTCGTGTCCA | | |
| | | | ATTCCTATCGTGATTATCTTGAAGTGCATGGCATCGGT | | |
| | | | GTGCAGCTAACCGAGGCGCTGGCCGAATACTGGCACCG | | |
| | | | GCGCATTCGTGAAGAGCTGAAATTCTCCGGAAACCGGA | | |
| | | | CGATGTCGGCTGACGATCCCGAGGCCGTCGAGGACTAT | | |
| | | | TTCAAGCTCGGCTACCGAGGTGCCCGCTTCGCGTTCGG | | |
| | | | GTATGGAGCATGCCCGGACCTGGAGGACCGGATCAAGA | | |
| | | | TGATGGAGCTGCTTCAACCCGAACGCATCGGTGTAACG | | |
| | | | ATATCTGAAGAGTTGCAGTTACATCCCGAGCAATCGAC | | |
| | | | TGATGCGTTCGTGCTGCACCATCCGGCGGCTAAGTACT | | |
| | | | TCAACGTCTGA | | |
| metH | Lactobacillus | AL935256 | ATGAAGTTTAAACAAGCACTCCAGCAACGGGTCCTCGT | 145 | |
| | plantarum | | TGCCGATGGCGCAATGGGCACCCTTTTATATGGTAACT | | |
| | | | ATGGCATCAATTCGGCTTTTGAAAACCTGAATTTGACG | | |
| | | | CATCCCGACACGATCTTACGCGTTCACCGATCGTACAT | | |
| | | | TCGGGCTGGTGCCGATATTATTCAAACCAACACCTACG | | |
| | | | CTGCGAACCGCCTAAAGTTGACCCGGTATGATTTACAA | | |
| | | | GACCAAGTCACCACCATCAATCAGGCCGCTGTGAAAAT | | |
| | | | TGCAGCGACCGCACGGGAACACGCGGATCACCCCGTTT | | |
| | | | ACATTCTGGGAACGATCGGTGGACTAGCCGGCGATACC | | |
| | | | GATGCAACTGTTCAACGGGCGACACCAGCAACGATTGC | | |
| | | | TGCCAGCGTGACTGAACAACTTACCGCCCTTCTAGCCA | | |
| | | | CCAACCAGTTAGATGGCATCTTGCTCGAAACATATTAT | | |
| | | | GATTTGCCAGAACTACTCGCCGCGTTAAAAATCGTGAA | | |
| | | | GGCCCATACTGACTTGCCCGTCATCACGAATGTTTCAA | | |
| | | | TGTTAGCCCCCGGCGTCTTACGAAACGGTACGAGCTTC | | |
| | | | ACTGATGCCATCGTCCAACTCAACGCTGCCGGCGCCGA | | |
| | | | CGTAATCGGCACGAACTGTCGCCTGGGACCTTACTATT | | |
| | | | TAGCTCAGTCATTTGAAAACTTGGCGATTCCAGCTAAC | | |
| | | | GTTAAACTAGCCGTTTACCCAAACGCTGGCTTGCCTGG | | |
| | | | CACTGATCAGGACGGTGCGGTGGTCTACGATGGTGAAC | | |
| | | | CAAGCTATTTCGAAGAATATGCCGAACGCTTTCGTCAG | | |
| | | | CTCGGTCTGAACATTATTGGTGGTTGTTGTGGGACCAC | | |
| | | | ACCTTTGCATACCAGCGCAACCGTCCGCGGTCTAAGTA | | |
| | | | ATCGCAGCATCGTTGCTCATGACCAGCCGGCTACAAAA | | |
| | | | CCACAGCCACCAACGCTCGTCACGACAAAGAGTCAGCA | | |
| | | | CCGGTTTCTGCAAAAAGTTGCGACCCAAAAAACGGCGT | | |
| | | | TAGTCGAACTCGATCCACCCCGCGATTTTGATACGACT | | |
| | | | AAATTTTTCCGGGGTGCTGAACGATTAAAAGCCGCTGG | | |
| | | | TGTCGATGGCATTACACTGTCTGACAATTCGTTAGCAA | | |
| | | | CGGTCCGGATTGCTAATACGACGATTGCGGCGCAGCTC | | |
| | | | AAGTTGAACTACGGCATCACGCCGATCGTTCACTTGAC | | |
| | | | GACCCGCGACCACAATCTAATCGGCTTACAATCAGAGA | | |
| | | | TCATGGGTCTACACAGCCTGGGTATTGAGGACATCTTA | | |
| | | | GCTATCACTGGCGATCCGGCCAAACTCGGTGATTTTCC | | |
| | | | GGGAGCCACTTCGGTCAGCGATGTGCGCTCCGTTGAAC | | |
| | | | TGATGAAGTTGATCAAGCAATTCAATAGCGGCATCGGA | | |
| | | | CCAACGGGTAAGTCGCTTAAAGAAGCCAGTGACTTTCG | | |
| | | | GGTCGCAGGCGCCTTTAATCCTAACGCTTATCGCACTT | | |
| | | | CCATATCGACCAAGTCAATCAGTCGGAAGTTAAGTTAT | | |
| | | | GGTTGTGACTACATTATCACCCAACCCGTGTATGATCT | | |
| | | | TGCAAACGTTGACGCTTTGGCGGATGCTCTAGCGGCTA | | |
| | | | ATCACGTGAATGTGCCAGTGTTCGTTGGTGTTATGCCA | | |
| | | | CTCGTCTCACGGCGTAATGCTGAATTTCTACACCATGA | | |
| | | | AGTCCATGGCATTCGGATTCCAGAGCCTATCTTGACAC | | |
| | | | GCATGGCAGAAGCCGAACAGACCGGAAACGAACGGGCA | | |
| | | | GTGGGCATTGCTATTGCAAAGGAATTGATTGATGGTAT | | |
| | | | CTGTGCGCGCTTCAACGGCGTTCACATCGTCACACCGT | | |
| | | | TTAACCGCTTTAAAACGGTCATTGAATTAGTCGATTAC | | |
| | | | ATCCAACAGAAAAACTTAATTAAAGTACAATAA | | |
| metH | Coryne- | AX371329 | ATGTCTACTTCAGTTACTTCACCAGCCCACAACAACGC | 260 | |
| | bacterium | | ACATTCCTCCGAATTTTTGGATGCGTTGGCAAACCATG | | |
| | glutamicum | | TGTTGATCGGCGACGGCGCCATGGGCACCCAGCTCCAA | | |
| | | | GGCTTTGACCTGGACGTGGAAAAGGATTTCCTTGATCT | | |
| | | | GGAGGGGTGTAATGAGATTCTCAACGACACCCGCCCTG | | |
| | | | ATGTGTTGAGGCAGATTCACCGCGCCTACTTTGAGGCG | | |
| | | | GGAGCTGACTTGGTTGAGACCAATACTTTTGGTTGCAA | | |
| | | | CCTGCCGAACTTGGCGGATTATGACATCGCTGATCGTT | | |
| | | | GCCGTGAGCTTGCCTACAAGGGCACTGCAGTGGCTAGG | | |
| | | | GAAGTGGCTGATGAGATGGGGCCGGGCCGAAACGGCAT | | |
| | | | GCGGCGTTTCGTGGTTGGTTCCCTGGGACCTGGAACGA | | |
| | | | AGCTTCCATCGCTGGGCCATGCACCGTATGCAGATTTG | | |
| | | | CGTGGGCACTACAAGGAAGCAGCGCTTGGCATCATCGA | | |
| | | | CGGTGGTGGCGATGCCTTTTTGATTGAGACTGCTCAGG | | |
| | | | ACTTGCTTCAGGTCAAGGCTGCGGTTCACGGCGTTCAA | | |
| | | | GATGCCATGGCTGAACTTGATACATTCTTGCCCATTAT | | |
| | | | TTGCCACGTCACCGTAGAGACCACCGGCACCATGCTCA | | |
| | | | TGGGTTCTGAGATCGGTGCCGCGTTGACAGCGCTGCAG | | |
| | | | CCACTGGGTATCGACATGATTGGTCTGAACTGCGCCAC | | |
| | | | CGGCCCAGATGAGATGAGCGAGCACCTGCGTTACCTGT | | |
| | | | CCAAGCACGCCGATATTCCTGTGTCGGTGATGCCTAAC | | |
| | | | GCAGGTCTTCCTGTCCTGGGTAAAAACGGTGCAGAATA | | |
| | | | CCCACTTGAGGCTGAGGATTTGGCGCAGGCGCTGGCTG | | |
| | | | GATTCGTCTCCGAATATGGCCTGTCCATGGTGGGTGGT | | |
| | | | TGTTGTGGCACCACACCTGAGCACATCCGTGCGGTCCG | | |
| | | | CGATGCGGTGGTTGGTGTTCCAGAGCAGGAAACCTCCA | | |
| | | | CACTGACCAAGATCCCTGCAGGCCCTGTTGAGCAGGCC | | |
| | | | TCCCGCGAGGTGGAGAAAGAGGACTCCGTCGCGTCGCT | | |
| | | | GTACACCTCGGTGCCATTGTCCCAGGAAACCGGCATTT | | |
| | | | CCATGATCGGTGAGCGCACCAACTCCAACGGTTCCAAG | | |
| | | | GCATTCCGTGAGGCAATGCTGTCTGGCGATTGGGAAAA | | |
| | | | GTGTGTGGATATTGCCAAGCAGCAAACCCGCGATGGTG | | |
| | | | CACACATGCTGGATCTTTGTGTGGATTACGTGGGACGA | | |
| | | | GACGGCACCGCCGATATGGCGACCTTGGCAGCACTTCT | | |
| | | | TGCTACCAGCTCCACTTTGCCAATCATGATTGACTCCA | | |
| | | | CCGAGCCAGAGGTTATTCGCACAGGCCTTGAGCACTTG | | |
| | | | GGTGGACGAAGCATCGTTAACTCCGTCAACTTTGAAGA | | |
| | | | CGGCGATGGCCCTGAGTCCCGCTACCAGCGCATCATGA | | |
| | | | AACTGGTAAAGCAGCACGGTGCGGCCGTGGTTGCGCTG | | |
| | | | ACCATTGATGAGGAAGGCCAGGCACGTACCGCTGAGCA | | |
| | | | CAAGGTGCGCATTGCTAAACGACTGATTGACGATATCA | | |
| | | | CCGGCAGCTACGGCCTGGATATCAAAGACATCGTTGTG | | |
| | | | GACTGCCTGACCTTCCCGATCTCTACTGGCCAGGAAGA | | |
| | | | AACCAGGCGAGATGGCATTGAAACCATCGAAGCCATCC | | |
| | | | GCGAGCTGAAGAAGCTCTACCCAGAAATCCACACCACC | | |
| | | | CTGGGTCTGTCCAATATTTCCTTCGGCCTGAACCCTGC | | |
| | | | TGCACGCCAGGTTCTTAACTCTGTGTTCCTCAATGAGT | | |
| | | | GCATTGAGGCTGGTCTGGACTCTGCGATTGCGCACAGC | | |
| | | | TCCAAGATTTTGCCGATGAACCGCATTGATGATCGCCA | | |
| | | | GCGCGAAGTGGCGTTGGATATGGTCTATGATCGCCGCA | | |
| | | | CCGAGGATTACGATCCGCTGCAGGAATTCATGCAGCTG | | |
| | | | TTTGAGGGCGTTTCTGCTGCCGATGCCAAGGATGCTCG | | |
| | | | CGCTGAACAGCTGGCCGCTATGCCTTTGTTTGAGCGTT | | |
| | | | TGGCACAGCGCATCATCGACGGCGATAAGAATGGCCTT | | |
| | | | GAGGATGATCTGGAAGCAGGCATGAAGGAGAAGTCTCC | | |
| | | | TATTGCGATCATCAACGAGGACCTTCTCAACGGCATGA | | |
| | | | AGACCGTGGGTGAGCTGTTTGGTTCCGGACAGATGCAG | | |
| | | | CTGCCATTCGTGCTGCAATCGGCAGAAACCATGAAAAC | | |
| | | | TGCGGTGGCCTATTTGGAACCGTTCATGGAAGAGGAAG | | |
| | | | CAGAAGCTACCGGATCTGCGCAGGCAGAGGGCAAGGGC | | |
| | | | AAAATCGTCGTGGCCACCGTCAAGGGTGACGTGCACGA | | |
| | | | TATCGGCAAGAACTTGGTGGACATCATTTTGTCCAACA | | |
| | | | ACGGTTACGACGTGGTGAACTTGGGCATCAAGCAGCCA | | |
| | | | CTGTCCGCCATGTTGGAAGCAGCGGAAGAACACAAAGC | | |
| | | | AGACGTCATCGGCATGTCGGGACTTCTTGTGAAGTCCA | | |
| | | | CCGTGGTGATGAAGGAAAACCTTGAGGAGATGAACAAC | | |
| | | | GCCGGCGCATCCAATTACCCAGTCATTTTGGGTGGCGC | | |
| | | | TGCGCTGACGCGTACCTACGTGGAAAACGATCTCAACG | | |
| | | | AGGTGTACACCGGTGAGGTGTACTACGCCCGTGATGCT | | |
| | | | TTCGAGGGCCTGCGCCTGATGGATGAGGTGATGGCAGA | | |
| | | | AAAGCGTGGTGAAGGACTTGATCCCAACTCACCAGAAG | | |
| | | | CTATTGAGCAGGCGAAGAAGAAGGCGGAACGTAAGGCT | | |
| | | | CGTAATGAGCGTTCCCGCAAGATTGCCGCGGAGCGTAA | | |
| | | | AGCTAATGCGGCTCCCGTGATTGTTCCGGAGCGTTCTG | | |
| | | | ATGTCTCCACCGATACTCCAACCGCGGCACCACCGTTC | | |
| | | | TGGGGAACCCGCATTGTCAAGGGTCTGCCCTTGGCGGA | | |
| | | | GTTCTTGGGCAACCTTGATGAGCGCGCCTTGTTCATGG | | |
| | | | GGCAGTGGGGTCTGAAATCCACCCGCGGCAACGAGGGT | | |
| | | | CCAAGCTATGAGGATTTGGTGGAAACTGAAGGCCGACC | | |
| | | | ACGCCTGCGCTACTGGCTGGATCGCCTGAAGTCTGAGG | | |
| | | | GCATTTTGGACCACGTGGCCTTGGTGTATGGCTACTTC | | |
| | | | CCAGCGGTCGCGGAAGGCGATGACGTGGTGATCTTGGA | | |
| | | | ATCCCCGGATCCACACGCAGCCGAACGCATGCGCTTTA | | |
| | | | GCTTCCCACGCCAGCAGCGCGGCAGGTTCTTGTGCATC | | |
| | | | GCGGATTTCATTCGCCCACGCGAGCAAGCTGTCAAGGA | | |
| | | | CGGCCAAGTGGACGTCATGCCATTCCAGCTGGTCACCA | | |
| | | | TGGGTAATCCTATTGCTGATTTCGCCAACGAGTTGTTC | | |
| | | | GCAGCCAATGAATACCGCGAGTACTTGGAAGTTCACGG | | |
| | | | CATCGGCGTGCAGCTCACCGAAGCATTGGCCGAGTACT | | |
| | | | GGCACTCCCGAGTGCGCAGCGAACTCAAGCTGAACGAC | | |
| | | | GGTGGATCTGTCGCTGATTTTGATCCAGAAGACAAGAC | | |
| | | | CAAGTTCTTCGACCTGGATTACCGCGGCGCCCGCTTCT | | |
| | | | CCTTTGGTTACGGTTCTTGCCCTGATCTGGAAGACCGC | | |
| | | | GCAAAGCTGGTGGAATTGCTCGAGCCAGGCCGTATCGG | | |
| | | | CGTGGAGTTGTCCGAGGAACTCCAGCTGCACCCAGAGC | | |
| | | | AGTCCACAGACGCGTTTGTGCTCTACCACCCAGAGGCA | | |
| | | | AAGTACTTTAACGTCTAA | | |
| metH | Escherichia coli | AE000475 | GTGAGCAGCAAAGTGGAACAACTGCGTGCGCAGTTAAA | 261 | |
| | | | TGAACGTATTCTGGTGCTGGACGGCGGTATGGGCACCA | | |
| | | | TGATCCAGAGTTATCGACTGAACGAAGCCGATTTTCGT | | |
| | | | GGTGAACGCTTTGCCGACTGGCCATGCGACCTCAAAGG | | |
| | | | CAACAACGACCTGCTGGTACTCAGTAAACCGGAAGTGA | | |
| | | | TCGCCGCTATCCACAACGCCTACTTTGAAGCGGGCGCG | | |
| | | | GATATCATCGAAACCAACACCTTCAACTCCACGACCAT | | |
| | | | TGCGATGGCGGATTACCAGATGGAATCCCTGTCGGCGG | | |
| | | | AAATCAACTTTGCGGCGGCGAAACTGGCGCGAGCTTGT | | |
| | | | GCTGACGAGTGGACCGCGCGCACGCCAGAGAAACCGCG | | |
| | | | CTACGTTGCCGGTGTTCTCGGCCCGACCAACCGCACGG | | |
| | | | CGTCTATTTCTCCGGACGTCAACGATCCGGCATTTCGT | | |
| | | | AATATCACTTTTGACGGGCTGGTGGCGGCTTATCGAGA | | |
| | | | GTCCACCAAAGCGCTGGTGGAAGGTGGCGCGGATCTGA | | |
| | | | TCCTGATTGAAACCGTTTTCGACACCCTTAACGCCAAA | | |
| | | | GCGGCGGTATTTGCGGTGAAAACGGAGTTTGAAGCGCT | | |
| | | | GGGCGTTGAGCTGCCGATTATGATCTCCGGCACCATCA | | |
| | | | CCGACGCCTCCGGGCGCACGCTCTCCGGGCAGACCACC | | |
| | | | GAAGCATTTTACAACTCATTGCGCCACGCCGAAGCTCT | | |
| | | | GACCTTTGGCCTGAACTGTGCGCTGGGGCCCGATGAAC | | |
| | | | TGCGCCAGTACGTGCAGGAGCTGTCACGGATTGCGGAA | | |
| | | | TGCTACGTCACCGCGCACCCGAACGCCGGGCTACCCAA | | |
| | | | CGCCTTTGGTGAGTACGATCTCGACGCCGACACGATGG | | |
| | | | CAAAACAGATACGTGAATGGGCGCAAGCGGGTTTTCTC | | |
| | | | AATATCGTCGGCGGCTGCTGTGGCACCACGCCACAACA | | |
| | | | TATTGCAGCGATGAGTCGTGCAGTAGAAGGATTAGCGC | | |
| | | | CGCGCAAACTGCCGGAAATTCCCGTAGCCTGCCGTTTG | | |
| | | | TCCGGCCTGGAGCCGCTGAACATTGGCGAAGATAGCCT | | |
| | | | GTTTGTGAACGTGGGTGAACGCACCAACGTCACCGGTT | | |
| | | | CCGCTAAGTTCAAGCGCCTGATCAAAGAAGAGAAATAC | | |
| | | | AGCGAGGCGCTGGATGTCGCGCGTCAACAGGTGGAAAA | | |
| | | | CGGCGCGCAGATTATCGATATCAACATGGATGAAGGGA | | |
| | | | TGCTCGATGCCGAAGCGGCGATGGTGCGTTTTCTCAAT | | |
| | | | CTGATTGCCGGTGAACCGGATATCGCTCGCGTGCCGAT | | |
| | | | TATGATCGACTCCTCAAAATGGGACGTCATTGAAAAAG | | |
| | | | GTCTGAAGTGTATCCAGGGCAAAGGCATTGTTAACTCT | | |
| | | | ATCTCGATGAAAGAGGGCGTCGATGCCTTTATCCATCA | | |
| | | | CGCGAAATTGTTGCGTCGCTACGGTGCGGCAGTGGTGG | | |
| | | | TAATGGCCTTTGACGAACAGGGACAGGCCGATACTCGC | | |
| | | | GCACGGAAAATCGAGATTTGCCGTCGGGCGTACAAAAT | | |
| | | | CCTCACCGAAGAGGTTGGCTTCCCGCCAGAAGATATCA | | |
| | | | TCTTCGACCCAAACATCTTCGCGGTCGCAACTGGCATT | | |
| | | | GAAGAGCACAACAACTACGCGCAGGACTTTATCGGCGC | | |
| | | | GTGTGAAGACATCAAACGCGAACTGCCGCACGCGCTGA | | |
| | | | TTTCCGGCGGCGTATCTAACGTTTCTTTCTCGTTCCGT | | |
| | | | GGCAACGATCCGGTGCGCGAAGCCATTCACGCAGTGTT | | |
| | | | CCTCTACTACGCTATTCGCAATGGCATGGATATGGGGA | | |
| | | | TCGTCAACGCCGGGCAACTGGCGATTTACGACGACCTA | | |
| | | | CCCGCTGAACTGCGCGACGCGGTGGAAGATGTGATTCT | | |
| | | | TAATCGTCGCGACGATGGCACCGAGCGTTTACTGGAGC | | |
| | | | TTGCCGAGAAATATCGCGGCAGCAAAACCGACGACACC | | |
| | | | GCCAACGCCCAGCAGGCGGAGTGGCGCTCGTGGGAAGT | | |
| | | | GAATAAACGTCTGGAATACTCGCTGGTCAAAGGCATTA | | |
| | | | CCGAGTTTATCGAGCAGGATACCGAAGAAGCCCGCCAG | | |
| | | | CAGGCTACGCGCCCGATTGAAGTGATTGAAGGCCCGTT | | |
| | | | GATGGACGGCATGAATGTGGTCGGCGACCTGTTTGGCG | | |
| | | | AAGGGAAAATGTTCCTGCCACAGGTGGTCAAATCGGCG | | |
| | | | CGCGTCATGAAACAGGCGGTGGCCTACCTCGAACCGTT | | |
| | | | TATTGAAGCCAGCAAAGAGCAGGGCAAAACCAACGGCA | | |
| | | | AGATGGTGATCGCCACCGTGAAGGGCGACGTCCACGAC | | |
| | | | ATCGGTAAAAATATCGTTGGTGTGGTGCTGCAATGTAA | | |
| | | | CAACTACGAAATTGTCGATCTCGGCGTTATGGTGCCTG | | |
| | | | CGGAAAAAATTCTCCGTACCGCTAAAGAAGTGAATGCT | | |
| | | | GATCTGATTGGCCTTTCGGGGCTTATCACGCCGTCGCT | | |
| | | | GGACGAGATGGTTAACGTGGCGAAAGAGATGGAGCGTC | | |
| | | | AGGGCTTCACTATTCCGTTACTGATTGGCGGCGCGACG | | |
| | | | ACCTCAAAAGCGCACACGGCGGTGAAAATCGAGCAGAA | | |
| | | | CTACAGCGGCCCGACGGTGTATGTGCAGAATGCCTCGC | | |
| | | | GTACCGTTGGTGTGGTGGCGGCGCTGCTTTCCGATACC | | |
| | | | CAGCGTGATGATTTTGTCGCTCGTACCCGCAAGGAGTA | | |
| | | | CGAAACCGTACGTATTCAGCACGGGCGCAAGAAACCGC | | |
| | | | GCACACCACCGGTCACGCTGGAAGCGGCGCGCGATAAC | | |
| | | | GATTTCGCTTTTGACTGGCAGGCTTACACGCCGCCGGT | | |
| | | | GGCGCACCGTCTCGGCGTGCAGGAAGTCGAAGCCAGCA | | |
| | | | TCGAAACGCTGCGTAATTACATCGACTGGACACCGTTC | | |
| | | | TTTATGACCTGGTCGCTGGCCGGGAAGTATCCGCGCAT | | |
| | | | TCTGGAAGATGAAGTGGTGGGCGTTGAGGCGCAGCGGC | | |
| | | | TGTTTAAAGACGCCAACGACATGCTGGATAAATTAAGC | | |
| | | | GCCGAGAAAACGCTGAATCCGCGTGGCGTGGTGGGCCT | | |
| | | | GTTCCCGGCAAACCGTGTGGGCGATGACATTGAAATCT | | |
| | | | ACCGTGACGAAACGCGTACCCATGTGATCAACGTCAGC | | |
| | | | CACCATCTGCGTCAACAGACCGAAAAAACAGGCTTCGC | | |
| | | | TAACTACTGTCTCGCTGACTTCGTTGCGCCGAAGCTTT | | |
| | | | CTGGTAAAGCAGATTACATCGGCGCATTTGCCGTGACT | | |
| | | | GGCGGGCTGGAAGAGGACGCACTGGCTGATGCCTTTGA | | |
| | | | AGCGCAGCACGATGATTACAACAAAATCATGGTGAAAG | | |
| | | | CGCTTGCCGACCGTTTAGCCGAAGCCTTTGCGGAGTAT | | |
| | | | CTCCATGAGCGTGTGCGTAAAGTCTACTGGGGCTATGC | | |
| | | | GCCGAACGAGAACCTCAGCAACGAAGAGCTGATCCGCG | | |
| | | | AAAACTACCAGGGCATCCGTCCGGCACCGGGCTATCCG | | |
| | | | GCCTGCCCGGAACATACGGAAAAAGCCACCATCTGGGA | | |
| | | | GCTGCTGGAAGTGGAAAAACACACTGGCATGAAACTCA | | |
| | | | CAGAATCTTTCGCCATGTGGCCCGGTGCATCGGTTTCG | | |
| | | | GGTTGGTACTTCAGCCACCCGGACAGCAAGTACTACGC | | |
| | | | TGTAGCACAAATTCAGCGCGATCAGGTTGAAGATTATG | | |
| | | | CCCGCCGTAAAGGTATGAGCGTTACCGAAGTTGAGCGC | | |
| | | | TGGCTGGCACCGAATCTGGGGTATGACGCGGACTGA | | |
| metE | Mycobacterium | Z95585.1 | GTGACCCAGCCTGTACGTCGTCAACCCTTTACCGCAAC | 146 | |
| | tuberculosis | | CATCACCGGCTCCCCGCGCATCGGCCCGCGCCGCGAAC | | |
| | (use this to | | TCAAGCGCGCCACCGAAGGCTACTGGGCCGGACGTACC | | |
| | clone M. | | AGCCGATCCGAGCTGGAGGCCGTCGCCGCCACGTTACG | | |
| | smegmatis | | CCGCGACACCTGGTCGGCCCTGGCCGCGGCCGGTCTGG | | |
| | gene) | | ACTCGGTGCCGGTGAACACCTTCTCCTACTACGACCAA | | |
| | | | ATGCTCGATACCGCGGTGCTGCTCGGCGCGCTGCCGCC | | |
| | | | CCGAGTGAGCCCGGTTTCCGACGGGCTGGACCGCTATT | | |
| | | | TCGCCGCGGCGCGGGGCACCGACCAGATCGCGCCGCTG | | |
| | | | GAGATGACGAAGTGGTTCGACACCAACTACCACTACCT | | |
| | | | GGTACCCGAGATCGGGCCGTCGACCACGTTCACGCTGC | | |
| | | | ACCCCGGCAAGGTGCTCGCCGAACTCAAAGAGGCGTTA | | |
| | | | GGGCAAGGCATTCCCGCACGTCCGGTGATCATCGGGCC | | |
| | | | GATCACCTTCCTGCTGCTGAGCAAGGCCGTCGACGGCG | | |
| | | | CGGGGGCGCCGATCGAACGCCTCGAAGAGTTGGTTCCG | | |
| | | | GTCTATTCGGAGCTGCTGTCGCTGCTTGCCGACGGCGG | | |
| | | | CGCCCAGTGGGTGCAGTTCGACGAGCCGGCGCTGGTGA | | |
| | | | CCGACCTCTCCCCCGACGCGCCCGCCCTGGCTGAAGCG | | |
| | | | GTGTACACCGCGCTGTGCTCGGTGAGCAACCGGCCTGC | | |
| | | | GATCTATGTCGCCACCTACTTCGGGGACCCGGGCGCGG | | |
| | | | CCCTACCGGCGCTGGCTCGCACCCCGGTCGAAGCCATC | | |
| | | | GGCGTCGACCTGGTGGCCGGTGCCGACACCTCGGTGGC | | |
| | | | CGGGGTACCCGAGCTGGCCGGCAAGACGCTGGTGGCCG | | |
| | | | GGGTCGTCGACGGGCGCAACGTCTGGCGCACCGACCTG | | |
| | | | GAGGCGGCGTTGGGCACGTTGGCGACCCTGCTGGGTTC | | |
| | | | GGCGGCTACCGTGGCCGTCTCGACGTCGTGCTCGACAC | | |
| | | | TGCACGTGCCGTACTCGCTGGAACCGGAAACCGACCTG | | |
| | | | GATGACGCGTTGCGGAGCTGGCTGGCGTTCGGTGCCGA | | |
| | | | AAAGGTGCGCGAAGTCGTCGTTCTCGCGCGTGCCCTGC | | |
| | | | GCGACGGACACGACGCGGTCGCCGACGAGATCGCGTCG | | |
| | | | TCCCGCGCCGCCATCGCGTCCCGCAAGCGCGACCCGCG | | |
| | | | GTTACACAATGGGCAAATCCGGGCGCGCATCGAGGCGA | | |
| | | | TCGTCGCGTCCGGAGCCCACCGCGGCAATGCCGCCCAG | | |
| | | | CGCCGCGCCAGCCAAGACGCGCGACTGCACCTGCCGCC | | |
| | | | GCTGCCGACCACGACGATCGGCTCCTACCCGCAGACCT | | |
| | | | CGGCGATCCGCGTTGCGCGTGCGGCGCTGCGGGCCGGT | | |
| | | | GAGATCGACGAGGCCGAGTACGTGCGCCGGATGCGGCA | | |
| | | | AGAGATCACCGAGGTGATCGCGCTACAGGAGCGGCTCG | | |
| | | | GGCTCGACGTGCTGGTGCACGGCGAACCGGAGCGCAAC | | |
| | | | GACATGGTGCAGTACTTCGCCGAGCAATTGGCGGGTTT | | |
| | | | CTTCGCTACCCAGAACGGCTGGGTGCAGTCCTACGGCA | | |
| | | | GCCGCTGTGTGCGTCCGCCGATCCTGTACGGCGACGTG | | |
| | | | TCCCGGCCGCGGGCGATGACGGTCGAGTGGATCACCTA | | |
| | | | CGCGCAGTCGCTGACCGACAAACCGGTGAAGGGCATGT | | |
| | | | TGACCGGGCCGGTGACGATTCTGGCGTGGTCGTTCGTG | | |
| | | | CGTGACGACCAGCCGTTGGCCGATACCGCCAACCAGGT | | |
| | | | GGCGCTGGCGATTCGCGACGAGACCGTGGATTTGCAGT | | |
| | | | CCGCCGGCATCGCGGTCATCCAGGTCGACGAGCCTGCG | | |
| | | | CTGCGTGAACTGCTGCCGCTGCGTCGCGCCGACCAGGC | | |
| | | | CGAGTACTTGCGTTGGGCGGTAGGGGCTTTCCGGTTGG | | |
| | | | CCACCTCCGGCGTCTCGGACGCCACCCAGATCCACACG | | |
| | | | CATCTGTGCTACTCGGAGTTCGGCGAGGTGATCGGCGC | | |
| | | | GATCGCCGATCTGGACGCGGACGTCACGTCCATCGAGG | | |
| | | | CGGCCCGGTCACACATGGAGGTGCTCGACGACCTGAAC | | |
| | | | GCGATCGGCTTCGCCAACGGTGTGGGCCCGGGCGTCTA | | |
| | | | TGACATTCACTCGCCACGGGTGCCCTCCGCTGAGGAGA | | |
| | | | TGGCCGACTCGTTGCGGGCCGCGTTGCGCGCGGTGCCG | | |
| | | | GCCGAGCGGCTGTGGGTCAACCCCGACTGCGGACTGAA | | |
| | | | GACCCGCAATGTCGACGAGGTGACCGCGTCGCTGCACA | | |
| | | | ACATGGTCGCCGCCGCCCGGGAGGTGCGCGCGGGCTAG | | |
| metE | Mycobacterium | Z94723.1 | ATGGACGAACTCGTGACCACTCAATCATTCACCGCAAC | 147 | |
| | leprae (use this | | CGTAACTGGCTCTCCACGCATTGGCCCGCGCCGCGAAC | | |
| | to clone M. | | TTAAACGGGCGACCGAAGGCTATTGGGCCAAGCGTACC | | |
| | smegmatis | | AGCCGATCAGAACTGGAGTCCGTCGCCTCAACATTGCG | | |
| | gene) | | CCGCGACATGTGGTCGGACTTAGCCGCCGCCGGCCTGG | | |
| | | | ACTCCGTACCGGTGAACACCTTCTCTTACTACGACCAG | | |
| | | | ATGCTCGACACGGCATTCATGCTCGGCGCGCTGCCTGC | | |
| | | | CCGGGTAGCACAAGTGTCCGACGACCTAGATCAGTACT | | |
| | | | TCGCCCTCGCACGCGGCAACAACGACATCAAGCCGCTG | | |
| | | | GAGATGACTAAGTGGTTCGACACCAACTACCACTACCT | | |
| | | | GGTTCCTGAAATCGAGCCCGCGACCACCTTCTCACTGA | | |
| | | | ACCCAGGCAAGATACTCGGTGAGCTGAAAGAAGCACTT | | |
| | | | GAGCAAAGAATTCCGTCCCGACCGGTCATTATCGGTCC | | |
| | | | GGTCACCTTCCTGTTACTGAGCAAGGGCATCAATGGCG | | |
| | | | GGGGCGCACCGATACAGCGGCTCGAGGAGCTGGTGGGA | | |
| | | | ATCTACTGCACGCTGCTATCACTGCTCGCCGAGAATGG | | |
| | | | CGCACGATGGGTACAGTTCGACGAGCCGGCGCTGGTGA | | |
| | | | CTGATCTATCCCCCGATGCACCGGCGTTGGCGGAAGCA | | |
| | | | GTTTACACTGCACTCGGCTCAGTTAGCAAACGACCCGC | | |
| | | | CATTTACGTGGCCACTTACTTCGGTAACCCCGGCGCTT | | |
| | | | CCTTGGCGGGGCTAGCCCGCACGCCCATCGAGGCGATC | | |
| | | | GGTGTCGACTTCGTTTGTGGTGCCGACACGTCGGTCGC | | |
| | | | GGCGGTGCCCGAGCTGGCCGGCAAGACTCTGGTGGCTG | | |
| | | | GCATCGTCGACGGACGCAACATCTGGCGCACTGACCTG | | |
| | | | GAATCGGCGTTGAGCAAGTTGGCTACTCTGCTGGGTTC | | |
| | | | AGCAGCCACCGTTGCTGTTTCGACGTCGTGCTCTACGC | | |
| | | | TGCATGTGCCGTATTCGTTGGAACCAGAAACCGACCTG | | |
| | | | GACGACAATTTGCGCAGCTGGCTGGCGTTCGGTGCGGA | | |
| | | | AAAGGTGGCCGAAGTCGTTGTGCTGGCACGCGCACTTC | | |
| | | | GCGACGGGCGCGACGCGGTCGCCGATGAGATCGCGGCG | | |
| | | | TCCAATGCCGCCGTTGCCTCGCGACGCAGCGACCCGCG | | |
| | | | GCTGCACAACGGGCAGGTACGCGCGCGTATTGACTCGA | | |
| | | | TTGTCGCTTCCGGTACGCACCGCGGTGACGCAGCGCAG | | |
| | | | CGCCGCACCAGCCAGGACGCGCGCCTACACTTACCGCC | | |
| | | | GCTGCCGACCACGACGATCGGCTCCTACCCGCAGACCT | | |
| | | | CAGCGATCCGCAAAGCGCGAGCGGCACTGCAGGACGCT | | |
| | | | GAGATCGACGAGGCCGAGTACATCAGCAGGATGAAAAA | | |
| | | | AGAAGTCGCCGACGCCATCAAACTGCAGGAGCAACTCG | | |
| | | | GGCTAGATGTACTGGTCCATGGCGAGCCGGAGCGCAAC | | |
| | | | GACATGGTACAGTATTTCGCTGAGCAACTGGGCGGCTT | | |
| | | | CTTCGCCACGCAGAACGGTTGGGTGCAGTCCTACGGCA | | |
| | | | GCCGTTGTGTACGTCCGCCGATCCTCTACGGTGACGTG | | |
| | | | TCCCGGCCTCACCCGATGACAATCGAGTGGATCACCTA | | |
| | | | CGCGCAGTCCCTAACTGACAAGCCAGTTAAGGGCATGT | | |
| | | | TGACCGGACCGGTCACGATCTTAGCCTGGTCGTTTGTT | | |
| | | | CGTGACGACCAGCCGCTGGCCGATACCGCGAACCAAGT | | |
| | | | AGCACTGGCGATTCGCGATGAGACCGTAGATCTACAAT | | |
| | | | CCGCCGGTATCGCAATCATCCAGGTTGACGAGCCCGCG | | |
| | | | CTACGTGAGCTGCTGCCGCTGCGTAGGGCTGATCAAGA | | |
| | | | CGAATACTTATGTTGGGCAGTAAAGGCTTTCCGCCTAG | | |
| | | | CTACCTCGGGGGTCGCCGACTCGACGCAAATCCACACT | | |
| | | | CATCTGTGCTACTCCGAGTTCGGCGAAGTGATTGGAGC | | |
| | | | TATCGCCGACCTGGACGCCGACGTCACATCCATCGAAG | | |
| | | | CGGCGCGCTCACACATGGAAGTATTGGATGACCTGAAC | | |
| | | | GCAGTCGGCTTCGCTAACAGCATAGGCCCGGGAGTCTA | | |
| | | | CGACATCCACTCGCCGCGGGTACCAAGCACTGACGAGA | | |
| | | | TTGCCAAGTCGCTACGCGCAGCATTAAAAGCCATACCG | | |
| | | | ATGCAACGGCTTTGGGTTAACCCCGACTGCGGGCTGAA | | |
| | | | GACCCGATCAGTTGACGAGGTGAGCGCGTCGCTGCAGA | | |
| | | | ACATGGTCGCAGCAGCACGCCAGGTGCGGGCAGGGGCC | | |
| | | | TAA | | |
| metE | Streptomyces | AL939107.1 | GTGACAGCGAAGTCCGCAGCCGCGGCAGCACGGGCCAC | 148 | |
| | coelicolor | | CGTGTACGGCTACCCCCGCCAGGGCCCGAACCGGGAAC | | |
| | | | TGAAGAAGGCGATCGAGGGCTACTGGAAGGGCCGCGTC | | |
| | | | AGCGCGCCCGAACTCCGGTCCCTCGCCGCGGACCTGCG | | |
| | | | CGCCGCGAACTGGCGCCGACTGGCCGACGCCGGCATCG | | |
| | | | ACGAGGTGCCCGCCGGCGACTTCTCGTACTACGACCAC | | |
| | | | GTCCTCGACACCACCGTCATGGTCGGTGCGATCCCCGA | | |
| | | | GCGCCACCGCGCCGCCGTCGCGGCCGACGCCCTGGACG | | |
| | | | GCTACTTCGCCATGGCCCGCGGCACCCAGGAGGTCGCG | | |
| | | | CCGCTGGAGATGACCAAGTGGTTCGACACCAACTACCA | | |
| | | | CTATCTGGTTCCGGAGTTGGGTCCGGACACCGTCTTCA | | |
| | | | CGGCCGACTCCACCAAGCAGGTCACCGAGCTGGCGGAA | | |
| | | | GCCGTCGCCCTGGGCCTGACCGCCCGCCCCGTGCTGGT | | |
| | | | CGGCCCGGTCACCTATCTCCTGCTGGCCAAGCCGGCCC | | |
| | | | CCGGCGCCCCCGCGGACTTCGAGCCGCTCACCCTGCTC | | |
| | | | GACCGGCTCCTGCCGGTGTACGCCGAGGTCCTCACCGA | | |
| | | | CCTGCGCGCGGCCGGCGCCGAGTGGGTCCAGCTGGACG | | |
| | | | AGCCCGCCTTCGTGCAGGACCGCACCCCGGCGGAACTG | | |
| | | | AACGCCCTGGAACGCGCCTACCGGGAACTCGGCGCCCT | | |
| | | | GACCGACCGGCCCAAGCTGCTCGTCGCCTCCTACTTCG | | |
| | | | ACCGCCTCGGCGACGCGCTGCCCGTCCTGGCCAAGGCA | | |
| | | | CCGATCGAGGGTCTTGCCCTGGACTTCACCGACGCCGC | | |
| | | | CGCGACCAACCTGGACGCCTTGGCCGCCGTCGGCGGAC | | |
| | | | TGCCCGGCAAGCGCCTCGTCGCCGGTGTCGTCAACGGC | | |
| | | | CGCAACATCTGGATCAACGACCTGCAGAAGTCGTTGTC | | |
| | | | CACGCTCGGCACGCTGCTGGGTCTCGCGGACCGGGTCG | | |
| | | | ACGTGTCCGCCTCCTGCTCCCTCCTCCATGTGCCCCTC | | |
| | | | GACACCGGGGCGGAGCGGGACATCGAGCCGCAGATCCT | | |
| | | | GCGCTGGCTGGCCTTCGCCCGGCAGAAGACCGCCGAGA | | |
| | | | TCGTCACCCTCGCCAAGGGCCTCGCCCAGGGCACCGAC | | |
| | | | GCCATCACCGGCGAACTCGCCGCCAGCCGCGCCGACAT | | |
| | | | GGCCTCCCGCGCCGGCTCACCGATCACCCGCAACCCGG | | |
| | | | CCGTACGAGCCCGTGCCGAGGCCGTGACGGACGACGAC | | |
| | | | GCCCGTCGCTCCCAGCCGTACGCCGAACGGACCGCCGC | | |
| | | | CCAGCGGGCACACCTGGGGCTGCCGCCGCTGCCGACCA | | |
| | | | CGACCATCGGCTCGTTCCCGCAGACCGGCGAGATCCGG | | |
| | | | GCCGCCCGTGCCGACCTGCGCGACGGCCGCATCGACAT | | |
| | | | CGCCGGCTACGAGGAACGGATCCGGGCCGAGATCCAGG | | |
| | | | AGGTGATCTCCTTCCAGGAGAAGACCGGCCTGGACGTC | | |
| | | | CTGGTGCACGGCGAGCCCGAACGCAACGACATGGTCCA | | |
| | | | GTACTTCGCCGAACAGCTGACCGGGTATCTGGCCACGC | | |
| | | | AGCACGGCTGGGTCCAGTCCTACGGCACCCGCTACGTC | | |
| | | | CGCCCGCCGATCCTGGCCGGGGACATCTCCCGCCCCGA | | |
| | | | GCCGATGACGGTGCGCTGGACGACGTACGCCCAGTCGC | | |
| | | | TCACCGAGAAGCCGGTCAAGGGCATGCTCACCGGCCCG | | |
| | | | GTGACCATGCTCGCATGGTCCTTCGTCCGCGACGACCA | | |
| | | | GCCCCTCGGTGACACCGCCCGCCAGGTCGCCCTCGCCC | | |
| | | | TGCGCGACGAGGTGAACGACCTGGAGGCGGCCGGGACC | | |
| | | | TCGGTCATCCAGGTCGACGAACCCGCCCTGCGCGAGAC | | |
| | | | ACTGCCGCTGCGGGCCGCCGACCACACCGCCTACCTGG | | |
| | | | CCTGGGCGACGGAGGCGTTCCGGCTGACCACCTCTGGC | | |
| | | | GTCCGCCCGGACACCCAGATCCACACCCACATGTGCTA | | |
| | | | CGCCGAGTTCGGCGACATCGTCCAGGCCATCGACGACC | | |
| | | | TCGACGCCGACGTCATCAGCCTGGAAGCCGCTCGCTCA | | |
| | | | CACATGCAGGTAGCCCACGAACTCGCTACCCACGGCTA | | |
| | | | CCCGCGCGAAGCCGGACCCGGCGTGTACGACATCCACT | | |
| | | | CCCCGCGCGTCCCGAGCGCCGAGGAAGCCGCCGCACTG | | |
| | | | CTGCGCACCGGCCTCAAGGCGATTCCTGCCGAACGGCT | | |
| | | | GTGGGTCAACCCCGACTGCGGTCTGAAGACCCGCGGCT | | |
| | | | GGCCCGAGACCCGCGCCTCCCTGGAGAACCTGGTCGCC | | |
| | | | ACCGCCCGCACCCTCCGCGGAGAGCTGTCCGCTTCCTGA | | |
| metE | Coryne- | AX371335 | ATGACTTCCAACTTTTCTTCCACTGTCGCTGGTCTTCC | 262 | |
| | bacterium | | TCGCATCGGAGCGAAGCGTGAACTGAAGTTCGCGCTCG | | |
| | glutamicum | | AAGGCTACTGGAATGGATCAATTGAAGGTCGCGAACTT | | |
| | | | CGGCAGACCGCCCGCCAATTGGTCAACACTGCATCGGA | | |
| | | | TTCTTTGTCTGGATTGGATTCCGTTCCGTTTGCAGGAC | | |
| | | | GTTCCTACTACGACGCAATGCTCGATACCGCCGCTATT | | |
| | | | TTGGGTGTGCTGCCGGAGCGTTTTGATGACATCGCTGA | | |
| | | | TCATGAAAACGATGGTCTCCCACTGTGGATTGACCGCT | | |
| | | | ACTTTGGCGCTGCTCGCGGTACTGAGACCCTGCCTGCA | | |
| | | | CAGGCAATGACCAAGTGGTTTGATACCAACTACCACTA | | |
| | | | CCTCGTGCCGGAGTTGTCTGCGGATACACGTTTCGTTT | | |
| | | | TGGATGCGTCCGCGCTGATTGAGGATCTCCGTTGCCAG | | |
| | | | CAGGTTCGTGGCGTTAATGCCCGCCCTGTTCTGGTTGG | | |
| | | | TCCACTGACTTTCCTTTCCCTTGCTCGCACCACTGATG | | |
| | | | GTTCCAATCCTTTGGATCACCTGCCTGCACTGTTTGAG | | |
| | | | GTCTACGAGCGCCTCATCAAGTCTTTCGATACTGAGTG | | |
| | | | GGTTCAGATCGATGAGCCTGCGTTGGTCACCGATGTTG | | |
| | | | CTCCTGAGGTTTTGGAGCAGGTCCGCGCTGGTTACACC | | |
| | | | ACTTTGGCTAAGCGCGATGGCGTGTTTGTCAATACTTA | | |
| | | | CTTCGGCTCTGGCGATCAGGCGCTGAACACTCTTGCGG | | |
| | | | GCATCGGCCTTGGCGCGATTGGCGTTGACTTGGTCACC | | |
| | | | CATGGCGTCACTGAGCTTGCTGCGTGGAAGGGTGAGGA | | |
| | | | GCTGCTGGTTGCGGGCATCGTTGATGGTCGTAACATTT | | |
| | | | GGCGCACCGACCTGTGTGCTGCTCTTGCTTCCCTGAAG | | |
| | | | CGCCTGGCAGCTCGCGGCCCAATCGCAGTGTCTACCTC | | |
| | | | TTGTTCACTGCTGCACGTTCCTTACACCCTCGAGGCTG | | |
| | | | AGAACATTGAGCCTGAGGTCCGCGACTGGCTTGCCTTC | | |
| | | | GGCTCGGAGAAGATCACCGAGGTCAAGCTGCTTGCCGA | | |
| | | | CGCCCTAGCCGGCAACATCGACGCGGCTGCGTTCGATG | | |
| | | | CGGCGTCCGCAGCAATTGCTTCTCGACGCACCTCCCCA | | |
| | | | CGCACCGCACCAATCACGCAGGAACTCCCTGGCCGTAG | | |
| | | | CCGTGGATCCTTCGACACTCGTGTTACGCTGCAGGAGA | | |
| | | | AGTCACTGGAGCTTCCAGCTCTGCCAACCACCACCATT | | |
| | | | GGTTCTTTCCCACAGACCCCATCCATTCGTTCTGCTCG | | |
| | | | CGCTCGTCTGCGCAAGGAATCCATCACTTTGGAGCAGT | | |
| | | | ACGAAGAGGCAATGCGCGAAGAAATCGATCTGGTCATC | | |
| | | | GCCAAGCAGGAAGAACTTGGTCTTGATGTGTTGGTTCA | | |
| | | | CGGTGAGCCAGAGCGCAACGACATGGTTCAGTACTTCT | | |
| | | | CTGAACTTCTCGACGGTTTCCTCTCAACCGCCAACGGC | | |
| | | | TGGGTCCAAAGCTACGGCTCCCGCTGTGTTCGTCCTCC | | |
| | | | AGTGTTGTTCGGAAACGTTTCCCGCCCAGCGCCAATGA | | |
| | | | CTGTCAAGTGGTTCCAGTACGCACAGAGCCTGACCCAG | | |
| | | | AAGCATGTCAAGGGAATGCTCACCGGTCCAGTCACCAT | | |
| | | | CCTTGCATGGTCCTTCGTTCGCGATGATCAGCCGCTGG | | |
| | | | CTACCACTGCTGACCAGGTTGCACTGGCACTGCGCGAT | | |
| | | | GAAATTAACGATCTCATCGAGGCTGGCGCGAAGATCAT | | |
| | | | CCAGGTGGATGAGCCTGCGATTCGTGAACTGTTGCCGC | | |
| | | | TACGAGACGTCGATAAGCCTGCCTACCTGCAGTGGTCC | | |
| | | | GTGGACTCCTTCCGCCTGGCGACTGCCGGCGCACCCGA | | |
| | | | CGACGTCCAAATCCACACCCACATGTGCTACTCCGAGT | | |
| | | | TCAACGAAGTGATCTCCTCGGTCATCGCGTTGGATGCC | | |
| | | | GATGTCACCACCATCGAAGCAGCACGTTCCGACATGCA | | |
| | | | GGTCCTCGCTGCTCTGAAATCTTCCGGCTTCGAGCTCG | | |
| | | | GCGTCGGACCTGGTGTGTGGGATATCCACTCCCCGCGC | | |
| | | | GTTCCTTCCGCGCAGGAAGTGGACGGTCTCCTCGAGGC | | |
| | | | TGCACTGCAGTCCGTGGATCCTCGCCAGCTGTGGGTCA | | |
| | | | ACCCAGACTGTGGTCTGAAGACCCGTGGATGGCCAGAA | | |
| | | | GTGGAAGCTTCCCTAAAGGTTCTCGTTGAGTCCGCTAA | | |
| | | | GCAGGCTCGTGAGAAAATCGGAGCAACTATCTAA | | |
| metE | Escherichia coli | AE016769 | ATGACAATTCTTAATCACACCCTCGGTTTCCCTCGCGT | 263 | |
| | | | TGGCCTGCGTCGCGAGCTGAAAAAAGCGCAAGAGAGTT | | |
| | | | ATTGGGCGGGGAACTCCACGCGTGAAGAACTGCTGGCG | | |
| | | | GTAGGGCGTGAATTGCGTGCTCGTCACTGGGATCAACA | | |
| | | | AAAGCAAGCGGGTATCGACCTGCTGCCGGTGGGCGATT | | |
| | | | TTGCCTGGTACGATCATGTACTGACCACCAGTCTGCTG | | |
| | | | CTGGGTAATGTTCCGCCACGTCATCAGAACAAAGATGG | | |
| | | | TTCGGTAGATATCGACACCCTGTTCCGTATTGGTCGTG | | |
| | | | GACGTGCACCGACTGGCGAACCTGCGGCGGCAGCGGAA | | |
| | | | ATGACCAAATGGTTTAACACCAACTATCACTACATGGT | | |
| | | | GCCGGAGTTCGTTAAAGGCCAACAGTTCAAACTGACCT | | |
| | | | GGACGCAGCTGCTGGAGGAAGTGGACGAGGCGCTGGCG | | |
| | | | CTGGGCCACAAGGTGAAACCTGTGCTGCTGGGGCCGAT | | |
| | | | TACCTACCTGTGGCTGGGTAAAGTGAAAGGTGAACAGT | | |
| | | | TTGATCGCCTGAGCCTGCTGAACGACATTCTGCCGGTT | | |
| | | | TATCAGCAAGTGCTGGCAGAACTGGCGAAACGCGGCAT | | |
| | | | CGAGTGGGTACAGATTGATGAACCCGCGTTGGTACTGG | | |
| | | | AACTGCCGCAGGCGTGGCTGGACGCATACAAACCCGCT | | |
| | | | TACGACGCGCTCCAGGGACAGGTGAAACTGCTGCTGAC | | |
| | | | CACCTATTTTGAAGGCGTAACGCCAAACCTCGACACGA | | |
| | | | TTACTGCGCTGCCTGTTCAGGGTCTGCATGTCGATCTc | | |
| | | | GTACATGGTAAAGATGACGTTGCTGAACTGCACAAGCG | | |
| | | | TCTGCCTTCTGACTGGCTGCTGTCTGCGGGTCTTATCA | | |
| | | | ATGGTCGTAACGTCTGGCGCGCCGATCTTACCGAGAAA | | |
| | | | TATGCGCAAATTAAGGACATTGTCGGCAAACGCGATTT | | |
| | | | GTGGGTGGCATCTTCCTGCTCGTTGCTGCACAGCCCCA | | |
| | | | TCGACTTGAGCGTGGAAACGCGTCTTGATGCAGAAGTG | | |
| | | | AAAAGCTGGTTTGCCTTCGCCCTGCAAAAATGTCATGA | | |
| | | | ACTGGCATTGCTGCGCGATGCGTTGAACAGTGGTGATA | | |
| | | | CGGCAGCTCTGGCAGAGTGGAGCGCTCCGATTCAGGCG | | |
| | | | CGTCGTCACTCTACTCGTGTACATAATCCGGCAGTAGA | | |
| | | | AAAGCGTCTGGCGGCGATCACCGCCCAGGACAGTCAGC | | |
| | | | GTGCGAATGTCTATGAAGTGCGTGCTGAAGCTCAGCGT | | |
| | | | GCGCGTTTTAAACTGCCCGCGTGGCCGACCACCACGAT | | |
| | | | TGGTTCCTTCCCGCAAACCACGGAGATTCGTACCCTGC | | |
| | | | GTCTGGATTTTAAAAAGGGTAATCTCGACGCCAATAAC | | |
| | | | TACCGCACGGGCATTGCGGAACATATCAAGCAGGCCAT | | |
| | | | TGTTGAGCAGGAACGTTTGGGACTGGATGTGCTGGTAC | | |
| | | | ATGGCGAGGCCGAGCGTAATGACATGGTGGAATACTTT | | |
| | | | GGCGAGCATCTGGATGGCTTTGTCTTTACGCAAAACGG | | |
| | | | TTGGGTACAGAGCTACGGTTCCCGCTGCGTGAAGCCAC | | |
| | | | CGATTGTTATTGGTGACGTTAGCCGCCCGGCACCGATT | | |
| | | | ACCGTGGAGTGGGCAAAATATGCGCAATCCCTGACTGA | | |
| | | | TAAACCGGTGAAAGGGATGTTGACCGGCCCGGTGACTA | | |
| | | | TTCTCTGCTGGTCGTTCCCGCGTGAAGATGTCAGCCGT | | |
| | | | GAAACCATCGCCAAACAAATTGCGCTGGCGCTGCGTGA | | |
| | | | TGAAGTCGCGGACCTGGAAGCCGCTGGAATTGGCATCA | | |
| | | | TTCAGATTGACGAACCGGCATTGCGCGAAGGTTTACCA | | |
| | | | CTGCGTCGCAGCGACTGGGATGCCTATCTCCAGTGGGG | | |
| | | | CGTGGAGGCTTTCCGTATCAACGCCGCCGTGGCGAAAG | | |
| | | | ATGACACACAAATCCACACTCACATGTGTTACTGCGAG | | |
| | | | TTCAACGACATCATGGATTCGATTGCGGCGCTGGACGC | | |
| | | | AGACGTCATCACCATCGAAACCTCGCGTTCCGACATGG | | |
| | | | AGTTGCTGGAGTCGTTTGAAGAGTTTGATTATCCAAAT | | |
| | | | GAAATCGGTCCTGGCGTCTATGACATTCACTCGCCAAA | | |
| | | | CGTACCGAGCGTGGAATGGATTGAAGCCTTGCTGAAGA | | |
| | | | AAGCGGCAAAACGCATTCCGGCAGAGCGTCTGTGGGTC | | |
| | | | AACCCGGACTGTGGCCTGAAAACGCGCGGCTGGCCAGA | | |
| | | | AACCCGCGCGGCACTGGCGAACATGGTGCAGGCGGCGC | | |
| | | | AGAATTTGCGTCGGGGA | | |
| glyA | Streptomyces | AL939123 | ATGTCGCTTCTGAACACACCCCTGCACGAGCTGGACCC | 149 | |
| | coelicolor | | GGACGTCGCCGCCGCCGTCGACGCCGAGCTGGACCGCC | | |
| | | | AGCAGTCCACCCTCGAGATGATCGCGTCGGAGAACTTC | | |
| | | | GCCCCGGTCGCGGTCATGGAGGCCCAGGGCTCGGTCCT | | |
| | | | CACCAACAAGTACGCCGAGGGCTACCCCGGCCGCCGCT | | |
| | | | ACTACGGCGGCTGCGAGCACGTCGACGTGGTCGAGCAG | | |
| | | | ATCGCCATCGACCGGGTCAAGGCGCTCTTCGGCGCCGA | | |
| | | | GCACGCCAACGTGCAGCCGCACTCGGGCGCCCAGGCCA | | |
| | | | ACGCGGCCGCGATGTTCGCGCTGCTCAAGCCCGGCGAC | | |
| | | | ACGATCATGGGTCTGAACCTCGCGCACGGCGGGCACCT | | |
| | | | GACCCACGGCATGAAGATCAACTTCTCCGGCAAGCTCT | | |
| | | | ACAACGTGGTCCCCTACCACGTCGGCGACGACGGCCAG | | |
| | | | GTCGACATGGCCGAGGTGGAGCGCCTGGCCAAGGAGAC | | |
| | | | CAAGCCGAAGCTGATCGTGGCGGGCTGGTCGGCCTACC | | |
| | | | CGCGTCAGCTGGACTTCGCCGCGTTCCGCAAGGTCGCG | | |
| | | | GACGAGGTCGGCGCGTACCTGATGGTCGACATGGCGCA | | |
| | | | CTTCGCCGGTCTGGTCGCGGCGGGCCTGCACCCGAACC | | |
| | | | CGGTCCCGCACGCCCACGTCGTCACCACGACCACCCAC | | |
| | | | AAGACGCTGGGCGGTCCGCGCGGCGGTGTGATCCTCTC | | |
| | | | CACGGCCGAGCTGGCCAAGAAGATCAACTCCGCCGTCT | | |
| | | | TCCCCGGTCAGCAGGGTGGCCCGCTGGAGCACGTGGTG | | |
| | | | GCCGCCAAGGCCGTCGCCTTCAAGGTCGCCGCGAGCGA | | |
| | | | GGACTTCAAGGAGCGCCAGGGCCGTACGCTGGAGGGTG | | |
| | | | CCCGCATCCTGGCCGAGCGCCTGGTGCGGGACGACGCG | | |
| | | | AAGGCCGCGGGCGTCTCCGTCCTGACCGGCGGCACGGA | | |
| | | | CGTCCACCTGGTCCTGGTGGACCTGCGCGACTCCGAGC | | |
| | | | TGGACGGACAGCAGGCCGAGGACCGCCTCCACGAGGTC | | |
| | | | GGCATCACGGTCAACCGCAACGCCGTCCCGAACGACCC | | |
| | | | GCGCCCGCCGATGGTGACCTCCGGTCTGCGCATCGGTA | | |
| | | | CGCCGGCCCTGGCGACCCGCGGCTTCACCGCCGAGGAC | | |
| | | | TTCGCCGAGGTCGCGGACGTGATCGCCGAGGCGCTGAA | | |
| | | | GCCGTCCTACGACGCGGAGGCCCTCAAGGCCCGGGTGA | | |
| | | | AGACCCTGGCCGACAAGCACCCGCTGTACCCGGGTCTG | | |
| | | | AACAAGTAG | | |
| glyA | Thermobifida | NZ_AAAQ010 | GTGAAGGTTAGGAAACTCATGACCGCCCAGAGCACTTC | 150 | |
| | fusca | 00038 | GCTCACCCAGTCGCTGGCTCAGCTCGACCCTGAGGTCG | | |
| | | | CGGCAGCCGTGGACGCCGAGCTCGCCCGCCAGCGCGAC | | |
| | | | ACCTTGGAGATGATCGCCTCCGAAAACTTTGCGCCCCG | | |
| | | | GGCGGTGCTGGAGGCGCAAGGCACGGTGCTGACCAACA | | |
| | | | AGTACGCGGAAGGCTACCCGGGCCGCCGCTACTACGGC | | |
| | | | GGGTGTGAGCACGTGGACGTCATCGAACAGCTGGCCAT | | |
| | | | CGACCGTGCCAAGGCCCTGTTCGGTGCCGAGCACGCCA | | |
| | | | ACGTGCAGCCGCACTCGGGCGCTCAGGCGAACACCGCC | | |
| | | | GTGTACTTTGCGCTGCTGCAGCCGGGCGACACCATCCT | | |
| | | | GGGCCTGGACCTCGCACACGGCGGGCACCTCACCCACG | | |
| | | | GCATGCGGATCAACTACTCCGGCAAGATCCTCAACGCC | | |
| | | | GTGGCCTACCACGTACGCGAGTCCGACGGCCTGATCGA | | |
| | | | CTACGACGAGGTCGAAGCGCTAGCCAAGGAGCACCAGC | | |
| | | | CGAAACTGATCATCGCGGGCTGGTCGGCGTACCCGCGC | | |
| | | | CAGTTGGACTTTGCCCGGTTCCGGGAGATCGCCGACCA | | |
| | | | GACAGGCGCCCTCCTCATGGTGGATATGGCGCATTTCG | | |
| | | | CGGGTCTGGTCGCGGCTGGACTGCACCCCAACCCGGTC | | |
| | | | CCCTACGCCGACGTAGTGACCACCACCACCCACAAGAC | | |
| | | | CTTGGGCGGGCCGCGAGGCGGGCTCATCCTGGCCAAGG | | |
| | | | AGGAGCTGGGCAAGAAGATCAACTCGGCGGTGTTCCCG | | |
| | | | GGGATGCAGGGCGGTCCGCTCCAGCACGTCATCGCTGC | | |
| | | | CAAGGCCGTAGCGTTGAAGGTCGCGGCCAGCGAAGAGT | | |
| | | | TCGCTGAGCGGCAGCGGCGCACCCTTTCCGGCGCGAAG | | |
| | | | ATCCTCGCCGAGCGGCTCACCCAGCCTGACGCGGCCGA | | |
| | | | GGCCGGTATTCGGGTGCTGACCGGCGGCACCGACGTCC | | |
| | | | ACCTGGTCCTGGTCGACCTGGTCAACTCGGAACTCAAC | | |
| | | | GGCAAAGAGGCGGAGGACCGGCTGCACGAGATCGGTAT | | |
| | | | CACGGTCAACCGCAACGCGGTCCCCAACGACCCGCGGC | | |
| | | | CGCCCATGGTCACGTCGGGACTGCGGATCGGCACCCCG | | |
| | | | GCTCTCGCCACCCGCGGTTTCGGCGACGCCGACTTCGC | | |
| | | | TGAGGTCGCCGACATCATCGCTGAGGCGCTCAAGCCGG | | |
| | | | GCTTCGACGCGGCGACCCTGCGCTCCCGCGTCCAGGCG | | |
| | | | CTGGCCGCCAAGCACCCGCTCTACCCTGGACTGTGA | | |
| glyA | Mycobacterium | E006993 | ATGTCTGCCCCGCTCGCTGAGGTTGACCCCGATATCGC | 151 | |
| | tuberculosis | | CGAGTTGCTGGCCAAGGAGCTTGGTCGGCAACGAGACA | | |
| | (use this to | | CCCTGGAGATGATCGCCTCGGAGAACTTCGCACCGCGC | | |
| | clone M. | | GCTGTGCTGCAGGCCCAGGGCAGTGTGCTGACCAACAA | | |
| | smegmatis | | GTACGCCGAGGGACTGCCCGGGCGGCGCTACTACGGCG | | |
| | gene) | | GTTGTGAGCACGTCGACGTGGTGGAAAACCTCGCCCGC | | |
| | | | GACCGAGCCAAGGCGTTGTTCGGTGCCGAATTCGCCAA | | |
| | | | TGTGCAACCGCATTCGGGCGCTCAGGCCAACGCCGCGG | | |
| | | | TGCTGCATGCGCTGATGTCACCCGGCGAGCGGCTGTTG | | |
| | | | GGTCTGGACCTGGCCAACGGTGGTCACCTGACCCATGG | | |
| | | | CATGCGGCTGAACTTCTCCGGCAAGCTCTACGAGAATG | | |
| | | | GCTTCTACGGCGTCGACCCGGCGACACATCTGATCGAC | | |
| | | | ATGGATGCGGTGCGGGCCACCGCACTCGAATTCCGCCC | | |
| | | | GAAGGTGATCATCGCCGGCTGGTCGGCCTACCCGCGGG | | |
| | | | TGCTCGACTTCGCGGCGTTCCGGTCGATCGCCGACGAG | | |
| | | | GTCGGGGCCAAGTTGCTCGTGGACATGGCGCATTTCGC | | |
| | | | GGGTCTGGTCGCCGCGGGGTTGCACCCGTCGCCGGTGC | | |
| | | | CGCACGCGGATGTGGTGTCCACCACCGTGCACAAGACG | | |
| | | | CTCGGCGGCGGCCGCTCCGGCCTGATCGTCGGTAAGCA | | |
| | | | GCAGTACGCCAAGGCGATCAACTCGGCGGTGTTTCCCG | | |
| | | | GGCAGCAGGGCGGTCCGCTCATGCACGTCATTGCCGGC | | |
| | | | AAGGCGGTCGCGTTGAAGATCGCCGCCACACCCGAATT | | |
| | | | TGCCGACCGGCAGCGGCGCACGCTGTCCGGGGCCCGGA | | |
| | | | TCATTGCCGATCGACTGATGGCTCCCGATGTCGCCAAG | | |
| | | | GCCGGTGTGTCGGTGGTCAGCGGCGGCACCGACGTCCA | | |
| | | | CCTGGTGCTGGTCGATCTGCGTGATTCCCCACTGGATG | | |
| | | | GCCAGGCCGCCGAGGACCTGCTGCACGAGGTCGGCATC | | |
| | | | ACGGTCAACCGCAACGCCGTCCCCAATGATCCCCGACC | | |
| | | | GCCGATGGTGACCTCGGGCCTGCGGATAGGCACGCCCG | | |
| | | | CGCTGGCGACCCGCGGCTTCGGCGACACCGAGTTCACC | | |
| | | | GAGGTCGCCGACATTATTGCGACCGCGCTGGCGACCGG | | |
| | | | CAGTTCCGTTGATGTGTCGGCGCTTAAGGATCGGGCGA | | |
| | | | CCCGGCTGGCCAGGGCGTTTCCGCTCTACGACGGGCTC | | |
| | | | GAGGAGTGGAGTCTGGTCGGCCGCTGA | | |
| glyA | Mycobacterium | AL049491 | ATGGTCGCGCCGCTGGCTGAAGTCGACCCGGATATCGC | 152 | |
| | leprae (use this | | CGAGCTACTGGGCAAAGAGCTAGGCCGGCAACGGGACA | | |
| | to clone M. | | CCTTGGAGATGATCGCTTCAGAGAACTTTGTGCCGCGC | | |
| | smegmatis | | TCGGTTCTACAGGCCCAAGGCAGCGTGCTGACCAACAA | | |
| | gene) | | GTACGCTGAGGGGTTGCCCGGCCGACGCTATTACGACG | | |
| | | | GCTGCGAGCACGTCGACGTCGTGGAGAACATCGCCCGC | | |
| | | | GACCGGGCCAAGGCGCTGTTCGGTGCCGACTTCGCCAA | | |
| | | | CGTGCAGCCGCACTCGGGGGCCCAGGCCAACGCCGCGG | | |
| | | | TACTGCACGCGCTGATGTCTCCGGGGGAGCGGCTGCTG | | |
| | | | GGTCTGGATCTCGCCAATGGCGGTCATCTGACGCATGG | | |
| | | | CATGCGGCTGAACTTCTCCGGCAAGCTGTATGAAACCG | | |
| | | | GCTTTTATGGCGTCGACGCGACAACGCATCTCATCGAT | | |
| | | | ATGGACGCGGTGCGGGCCAAGGCGCTCGAATTCCGCCC | | |
| | | | GAAGGTGCTGATCGCTGGCTGGTCGGCCTATCCGCGGA | | |
| | | | TTCTGGACTTCGCTGCTTTTCGGTCGATCGCAGACGAA | | |
| | | | GTCGGCGCCAAGCTGTGGGTCGACATGGCGCATTTCGC | | |
| | | | GGGCCTGGTTGCGGTGGGGTTGCACCCGTCTCCAGTGC | | |
| | | | CGCATGCAGATGTGGTGTCCACGACCGTTCACAAGACT | | |
| | | | CTTGGCGGGGGCCGTTCCGGTTTGATCCTGGGCAAGCA | | |
| | | | GGAGTTCGCCACGGCCATCAACTCAGCGGTGTTTCCTG | | |
| | | | GCCAGCAGGGTGGACCGCTTATGCATGTCATCGCGGGC | | |
| | | | AAGGCGGTCGCGCTGAAGATTGCTACCACGCCTGAGTT | | |
| | | | CACCGACCGGCAGCAGCGCACGCTGGCCGGCGCCCGGA | | |
| | | | TTCTCGCCGATCGGCTTACCGCCGCTGATGTCACCAAG | | |
| | | | GCCGGGGTGTCGGTGGTCAGTGGTGGCACTGACGTCCA | | |
| | | | CCTAGTGCTGGTCGACCTGCGCAACTCCCCGTTCGACG | | |
| | | | GCCAGGCAGCAGAAGATCTGCTGCACGAGGTCGGCATC | | |
| | | | ACTGTCAACCGCAACGTGGTTCCCAATGACCCCCGGCC | | |
| | | | GCCGATGGTGACCTCAGGCCTGCGGATAGGAACCCCCG | | |
| | | | CGCTGGCAACCCGAGGGTTCGGTGAAGCGGAGTTCACC | | |
| | | | GAGGTCGCGGACATCATCGCGACGGTGCTGACCACTGG | | |
| | | | TGGCAGTGTCGATGTGGCCGCGCTGCGGCAGCAGGTTA | | |
| | | | CCCGACTTGCCAGGGACTTCCCGCTCTACGGGGGACTT | | |
| | | | GAGGACTGGAGCTTGGCCGGTCGCTAG | | |
| glyA | Lactobacillus | AL935258 | ATGAATTACCAGGAACAAGATCCAGAAGTATGGGCTGC | 153 | |
| | plantarum | | GATTAGTAAGGAACAGGCACGGCAACAACATAATATTG | | |
| | | | AGTTGATTGCTTCTGAGAACATCGTTTCAAAGGGCGTC | | |
| | | | CGGGCAGCGCAGGGGAGTGTGCTGACCAATAAATACTC | | |
| | | | TGAAGGCTATCCGGGTCACCGCTTTTACGGTGGTAACG | | |
| | | | AATACATTGACCAAGTGGAAACCTTAGCAATTGAACGG | | |
| | | | GCTAAGAAATTATTTGGTGCGGAATATGCTAATGTGCA | | |
| | | | ACCACACTCTGGTTCCCAAGCCAATGCGGCTGCATATA | | |
| | | | TGGCACTGATTCAACCTGGTGACCGGGTGATGGGGATG | | |
| | | | TCACTAGATGCTGGGGGACACTTAACACATGGATCTAG | | |
| | | | TGTGAACTTCTCTGGTAAACTTTACGATTTTCAAGGTT | | |
| | | | ATGGGCTCGATCCTGAAACCGCAGAATTAAACTATGAT | | |
| | | | GCAATTCTTGCACAAGCACAAGATTTTCAACCAAAGTT | | |
| | | | AATCGTTGCGGGGGCTTCTGCTTATAGTCGATTGATTG | | |
| | | | ATTTCAAGAAGTTTCGCGAGATTGCAGATCAAGTTGGG | | |
| | | | GCCTTATTGATGGTTGATATGGCTCATATTGCCGGCTT | | |
| | | | AGTTGCGGCCGGGCTACATCCTAATCCAGTGCCATATG | | |
| | | | CTGATGTGGTTACGACAACGACGCACAAAACGTTACGG | | |
| | | | GGGCCCCGTGGCGGTATGATTTTAGCGAAAGAAAAGTA | | |
| | | | TGGCAAGAAGATCAACTCAGCCGTTTTCCCTGGCAATC | | |
| | | | AGGGTGGGCCGTTGGATCACGTAATTGCGGGTAAAGCG | | |
| | | | ATTGCTTTGGGCGAAGACTTACAGCCTGAGTTTAAGGT | | |
| | | | TTATGCCCAACATATCATTGATAATGCCAAGGCAATGG | | |
| | | | CGAAGGTCTTCAATGACTCTGACTTGGTTCGGGTTATT | | |
| | | | TCTGGTGGCACGGACAATCATTTAATGACGATTGATGT | | |
| | | | CACTAAGTCTGGTTTGAACGGTCGCCAAGTACAAGATC | | |
| | | | TGTTAGATACGGTTTATATTACGGTCAACAAAGAAGCG | | |
| | | | ATTCCGAATGAGACGTTAGGGGCTTTCAAGACCTCTGG | | |
| | | | TATTCGGTTGGGAACACCTGCGATTACGACCCGTGGTT | | |
| | | | TTGACGAAGCTGATGCAACTAAGGTCGCTGAATTGATT | | |
| | | | TTGCAAGCGTTACAAGCACCGACAGATCAAGCAAATCT | | |
| | | | AGATGACGTTAAACAGCAAGCAATGGCTTTAACAGCGA | | |
| | | | AGCACCCGATCGATGTTGATTAA | | |
| glyA | Corynebacterium | AF327063 | ATGACCGATGCCCACCAAGCGGACGATGTCCGTTACCA | 264 | |
| | glutamicum | | GCCACTGAACGAGCTTGATCCTGAGGTGGCTGCTGCCA | | |
| | | | TCGCTGGGGAACTTGCCCGTCAACGCGATACATTAGAG | | |
| | | | ATGATCGCGTCTGAGAACTTCGTTCCCCGTTCTGTTTT | | |
| | | | GCAGGCGCAGGGTTCTGTTCTTACCAATAAGTATGCCG | | |
| | | | AGGGTTACCCTGGCCGCCGTTACTACGGTGGTTGCGAA | | |
| | | | CAAGTTGACATCATTGAGGATCTTGCACGTGATCGTGC | | |
| | | | GAAGGCTCTCTTCGGTGCAGAGTTCGCCAATGTTCAGC | | |
| | | | CTCACTCTGGCGCACAGGCTAATGCTGCTGTGCTGATG | | |
| | | | ACTTTGGCTGAGCCAGGCGACAAGATCATGGGTCTGTC | | |
| | | | TTTGGCTCATGGTGGTCACTTGACCCACGGAATGAAGT | | |
| | | | TGAACTTCTCCGGAAAGCTGTACGAGGTTGTTGCGTAC | | |
| | | | GGTGTTGATCCTGAGACCATGCGTGTTGATATGGATCA | | |
| | | | GGTTCGTGAGATTGCTCTGAAGGAGCAGCCAAAGGTAA | | |
| | | | TTATCGCTGGCTGGTCTGCATACCCTCGCCACCTTGAT | | |
| | | | TTCGAGGCTTTCCAGTCTATTGCTGCGGAAGTTGGCGC | | |
| | | | GAAGCTGTGGGTCGATATGGCTCACTTCGCTGGTCTTG | | |
| | | | TTGCTGCTGGTTTGCACCCAAGCCCAGTTCCTTACTCT | | |
| | | | GATGTTGTTTCTTCCACTGTCCACAAGACTTTGGGTGG | | |
| | | | ACCTCGTTCCGGCATCATTCTGGCTAAGCAGGAGTACG | | |
| | | | CGAAGAAGCTGAACTCTTCCGTATTCCCAGGTCAGCAG | | |
| | | | GGTGGTCCTTTGATGCACGCAGTTGCTGCGAAGGCTAC | | |
| | | | TTCTTTGAAGATTGCTGGCACTGAGCAGTTCCGTGACc | | |
| | | | GTCAGGCTCGCACGTTGGAGGGTGCTCGCATTCTTGCT | | |
| | | | GAGCGTCTGACTGCTTCTGATGCGAAGGCCGCTGGCGT | | |
| | | | GGATGTCTTGACCGGTGGCACTGATGTGCACTTGGTTT | | |
| | | | TGGCTGATCTGCGTAACTCCCAGATGGATGGCCAGCAG | | |
| | | | GCGGAAGATCTGCTGCACGAGGTTGGTATCACTGTGAA | | |
| | | | CCGTAACGCGGTTCCTTTCGATCCTCGTCCACCAATGG | | |
| | | | TTACTTCTGGTCTGCGTATTGGTACTCCTGCGCTGGCT | | |
| | | | ACCCGTGGTTTCGATATTCCTGCATTCACTGAGGTTGC | | |
| | | | AGACATCATTGGTACTGCTTTGGCTAATGGTAAGTCCG | | |
| | | | CAGACATTGAGTCTCTGCGTGGCCGTGTAGCAAAGCTT | | |
| | | | GCTGCAGATTACCCACTGTATGAGGGCTTGGAAGACTG | | |
| | | | GACCATCGTCTAA | | |
| glyA | Escherichia coli | V00283 | ATGTTAAAGCGTGAAATGAACATTGCCGATTATGATGC | 265 | |
| | | | CGAACTGTGGCAGGCTATGGAGCAGGAAAAAGTACGTC | | |
| | | | AGGAAGAGCACATCGAACTGATCGCCTCCGAAAACTAC | | |
| | | | ACCAGCCCGCGCGTAATGCAGGCGCAGGGTTCTCAGCT | | |
| | | | GACCAACAAATATGCTGAAGGTTATCCGGGCAAACGCT | | |
| | | | ACTACGGCGGTTGCGAGTATGTTGATATCGTTGAACAA | | |
| | | | CTGGCGATCGATCGTGCGAAAGAACTGTTCGGCGCTGA | | |
| | | | CTACGCTAACGTCCAGCCGCACTCCGGCTCCCAGGCTA | | |
| | | | ACTTTGCGGTCTACACCGCGCTGCTGGAACCAGGTGAT | | |
| | | | ACCGTTCTGGGTATGAACCTGGCGCATGGCGGTCACCT | | |
| | | | GACTCACGGTTCTCCGGTTAACTTCTCCGGTAAACTGT | | |
| | | | ACAACATCGTTCCTTACGGTATCGATGCTACCGGTCAT | | |
| | | | ATCGACTACGCCGATCTGGAAAAACAAGCCAAAGAACA | | |
| | | | CAAGCCGAAAATGATTATCGGTGGTTTCTCTGCATATT | | |
| | | | CCGGCGTGGTGGACTGGGCGAAAATGCGTGAAATCGCT | | |
| | | | GACAGCATCGGTGCTTACCTGTTCGTTGATATGGCGCA | | |
| | | | CGTTGCGGGCCTGGTTGCTGCTGGCGTCTACCCGAACC | | |
| | | | CGGTTCCTCATGCTCACGTTGTTACTACCACCACTCAC | | |
| | | | AAAACCCTGGCGGGTCCGCGCGGCGGCCTGATCCTGGC | | |
| | | | GAAAGGTGGTAGCGAAGAGCTGTACAAAAAACTGAACT | | |
| | | | CTGCCGTTTTCCCTGGTGGTCAGGGCGGTCCGTTGATG | | |
| | | | CACGTAATCGCCGGTAAAGCGGTTGCTCTGAAAGAAGC | | |
| | | | GATGGAGCCTGAGTTCAAAACTTACCAGCAGCAGGTCG | | |
| | | | CTAAAAACGCTAAAGCGATGGTAGAAGTGTTCCTCGAG | | |
| | | | CGCGGCTACAAAGTGGTTTCCGGCGGCACTGATAACCA | | |
| | | | CCTGTTCCTGGTTGATCTGGTTGATAAAAACCTGACCG | | |
| | | | GTAAAGAAGCAGACGCCGCTCTGGGCCGTGCTAACATC | | |
| | | | ACCGTCAACAAAAACAGCGTACCGAACGATCCGAAGAG | | |
| | | | CCCGTTTGTGACCTCCGGTATTCGTGTAGGTACTCCGG | | |
| | | | CGATTACCCGTCGCGGCTTTAAAGAAGCCGAAGCGAAA | | |
| | | | GAACTGGCTGGCTGGATGTGTGACGTGCTGGACAGCAT | | |
| | | | CAATGATGAAGCCGTTATCGAGCGCATCAAAGGTAAAG | | |
| | | | TTCTCGACATCTGCGCACGTTACCCGGTTTACGCATAA | | |
| metE | Thermobifida | NZ_AAAQ010 | ATGGCTTCGAGGGCGGCCAGCACCGGTTCCCACTCCGC | 154 | |
| | fusca | 00010 | GCCGATCTCCAGCAGCAGCGGGCGTCGGCTCGCGACGA | | |
| | | | AGGCCGCCAGTTCGGCATCGACAAGGGGGCGCACGAAG | | |
| | | | GCGACGGGAGACAAGTGCGAGGAGCTCATAAGGGCAGG | | |
| | | | CTACCGATTGTTCCGCCGCCCGTCTTCACCACGACACA | | |
| | | | CCCAAACCCCACCGATATGGTCGATTACAGTGGGAGAC | | |
| | | | ATGCTCGGATCACCCACGCCGCGCCCGGCGCCTCGTCC | | |
| | | | GCGCCGTATCAGCGAACTGTTGGCGCGTAAAGAGCCCA | | |
| | | | CGTTCTCCTTCGAGTTCTTCCCCCCGAAAACGCCCGAG | | |
| | | | GGGGAGCGCATGCTTTGGCGGGCGATCCGGGAGATCGA | | |
| | | | GGCCCTACGCCCTTCCTTCGTCTCGGTGACCTACGGTG | | |
| | | | CGGGCGGCAGCACCCGGGACCGGACCGTGAACGTCACC | | |
| | | | GAGAAGATCGCCACCAACACCACTCTGCTGCCCGTGGC | | |
| | | | GCACATCACCGCGGTCAACCACTCGGTGCGGGAGCTCC | | |
| | | | GCCACCTCATCGGCCGGTTCGCGGCGGCGGGGGTGTGC | | |
| | | | AACATGCTCGCGCTGCGCGGCGACCCGCCCGGCGACCC | | |
| | | | GCTGGGCGAATGGGTCAAGCACCCGGAGGGCCTCACCC | | |
| | | | ACGCCGAAGAACTGGTGCGGCTGATCAAGGAGAGCGGT | | |
| | | | GACTTCTGCGTCGGGGTGGCCGCATTCCCCTACAAGCA | | |
| | | | CCCCCGCTCCCCCGACGTGGAGACCGACACGGACTTCT | | |
| | | | TCGTCCGCAAATGCCGGGCAGGAGCGGACTACGCGATC | | |
| | | | ACCCAGATGTTCTTCGAAGCCGAGGACTACCTGCGGCT | | |
| | | | GCGGGACCGGGTCGCGGCCCGGGGCTGCGACGTGCCCA | | |
| | | | TCATCCCTGAGATCATGCCGGTCACGAAGTTCAGCACG | | |
| | | | ATCGCCCGCTCCGAGCAGTTGTCGGGAGCGCCGTTCCC | | |
| | | | CCGCAGGCTGGCGGAAGAGTTCGAACGGGTCGCCGACG | | |
| | | | ACCCCGAGGCGGTGCGCGCGCTCGGTATCGAGCACGCC | | |
| | | | ACTCGGCTGTGCGAACGGCTCCTCGCCGAAGGGGCGCC | | |
| | | | GGGCATCCACTTCATCACGTTCAACCGTTCGACGGCGA | | |
| | | | CCCGCGAGGTCTACCACCGGCTCGTGGGCGCCACCCAG | | |
| | | | CCGGCAGCGGTAGCTGCGCTGCCATGA | | |
| metE | Streptomyces | AL939111 | ATGGCCCTCGGAACCGCAAGCACGAGGACGGATCGCGC | 155 | |
| | coelicolor | | CCGCACGGTGCGTGACATCCTCGCCACCGGCAAGACGA | | |
| | | | CGTACTCGTTCGAGTTCTCGGCGCCGAAGACGCCCAAG | | |
| | | | GGCGAGAGGAACCTCTGGAGCGCGCTGCGGCGGGTCGA | | |
| | | | GGCCGTGGCCCCGGACTTCGTCTCCGTGACCTACGGCG | | |
| | | | CCGGCGGCTCCACGCGCGCCGGCACGGTCCGCGAGACC | | |
| | | | CAGCAGATCGTCGCCGACACCACGCTGACCCCGGTGGC | | |
| | | | CCACCTCACCGCCGTCGACCACTCCGTCGCCGAGCTGC | | |
| | | | GCAACATCATCGGCCAGTACGCCGACGCCGGGATCCGC | | |
| | | | AACATGCTGGCCGTGCGCGGCGACCCGCCCGGCGACCC | | |
| | | | GAACGCCGACTGGATCGCGCACCCCGAGGGCCTGACCT | | |
| | | | ACGCGGCCGAACTGGTCAGGCTCATCAAGGAGTCGGGC | | |
| | | | GACTTCTGCGTCGGCGTCGCGGCCTTCCCCGAGATGCA | | |
| | | | CCCGCGCTCCGCCGACTGGGACACGGACGTCACGAACT | | |
| | | | TCGTCGACAAGTGCCGGGCCGGCGCCGACTACGCCATC | | |
| | | | ACCCAGATGTTCTTCCAGCCCGACTCCTATCTCCGGCT | | |
| | | | GCGCGACCGGGTCGCCGCGGCCGGCTGCGCGACCCCGG | | |
| | | | TCATCCCCGAGGTCATGCCGGTGACCAGTGTGAAGATG | | |
| | | | CTGGAGAGGTTGCCGAAGCTCAGCAACGCCTCGTTCCC | | |
| | | | GGCGGAGTTGAAAGAGCGGATCCTCACAGCCAAGGACG | | |
| | | | ATCCGGCGGCTGTACGCTCGATCGGCATCGAGTTCGCC | | |
| | | | ACGGAGTTCTGCGCGCGGCTGCTGGCCGAGGGAGTGCC | | |
| | | | AGGACTGCACTTCATCACGCTCAACAACTCCACGGCGA | | |
| | | | CGCTGGAAATCTACGAGAACCTGGGCCTGCACCACCCA | | |
| | | | CCGCGGGCCTAG | | |
| metE | Coryne- | AX374883 | TTGGTGGAGGTGAATAAATGCCAGAGGCAGTCCCAACA | 266 | |
| | bacterium | | AAACACTCTCATCACACTAAGATACCCAGGCATGTCCC | | |
| | glutamicum | | TAACGAACATCCCAGCCTCATCTCAATGGGCAATTAGC | | |
| | | | GACGTTTTGAAGCGTCCTTCACCCGGCCGAGTACCTTT | | |
| | | | TTCTGTCGAGTTTATGCCACCCCGCGACGATGCAGCTG | | |
| | | | AAGAGCGTCTTTACCGCGCAGCAGAGGTCTTCCATGAC | | |
| | | | CTCGGTGCATCGTTTGTCTCCGTGACTTATGGTGCTGG | | |
| | | | CGGATCAACCCGTGAGAGAACCTCACGTATTGCTCGAC | | |
| | | | GATTAGCGAAACAACCGTTGACCACTCTGGTGCACCTG | | |
| | | | ACCCTGGTTAACCACACTCGCGAAGAGATGAAGGCAAT | | |
| | | | TCTTCGGGAATACCTAGAGCTGGGATTAACAAACCTGT | | |
| | | | TGGCGCTTCGAGGAGATCCGCCTGGAGACCCATTAGGC | | |
| | | | GATTGGGTGAGCACCGATGGAGGACTGAACTATGCCTC | | |
| | | | TGAGCTCATCGATCTTATTAAGTCCACTCCTGAGTTCC | | |
| | | | GGGAATTCGACCTCGGTATCGCCTCCTTCCCCGAAGGG | | |
| | | | CATTTCCGGGCGAAAACTCTAGAAGAAGACACCAAATA | | |
| | | | CACTCTGGCGAAGCTGCGTGGAGGGGCAGAGTACTCCA | | |
| | | | TCACGCAGATGTTCTTTGATGTGGAAGACTACCTGCGA | | |
| | | | CTTCGTGATCGCCTTGTCGCTGCAGACCCCATTCATGG | | |
| | | | TGCGAAGCCAATCATTCCTGGCATCATGCCCATTACCG | | |
| | | | AGCTGCGGTCTGTGCGTCGACAGGTCGAACTCTCTGGT | | |
| | | | GCTCAATTGCCGAGCCAACTAGAAGAATCACTTGTTCG | | |
| | | | AGCTGCAAACGGCAATGAAGAAGCGAACAAAGACGAGA | | |
| | | | TCCGCAAGGTGGGCATTGAATATTCCACCAATATGGCA | | |
| | | | GAGCGACTCATTGCCGAAGGTGCGGAAGATCTGCACTT | | |
| | | | CATGACGCTTAACTTCACCCGTGCAACCCAAGAAGTGT | | |
| | | | TGTACAACCTTGGCATGGCGCCTGCTTGGGGAGCAGAG | | |
| | | | CACGGCCAAGACGCGGTGCGTTAA | | |
| metE | Escherichia coli | NC_000913 | ATGAGCTTTTTTCACGCCAGCCAGCGGGATGCCCTGAA | 267 | |
| | | | TCAGAGCCTGGCAGAAGTCCAGGGGCAGATTAACGTTT | | |
| | | | CGTTCGAGTTTTTCCCGCCGCGTACCAGTGAAATGGAG | | |
| | | | CAGACCCTGTGGAACTCCATCGATCGCCTTAGCAGCCT | | |
| | | | GAAACCGAAGTTTGTATCGGTGACCTATGGCGCGAACT | | |
| | | | CCGGCGAGCGCGACCGTACGCACAGCATTATTAAAGGC | | |
| | | | ATTAAAGATCGCACTGGTCTGGAAGCGGCACCGCATCT | | |
| | | | TACTTGCATTGATGCGACGCCCGACGAGCTGCGCACCA | | |
| | | | TTGCACGCGACTACTGGAATAACGGTATTCGTCATATC | | |
| | | | GTGGCGCTGCGTGGCGATCTGCCGCCGGGAAGTGGTAA | | |
| | | | GCCAGAAATGTATGCTTCTGACCTGGTGACGCTGTTAA | | |
| | | | AAGAAGTGGCAGATTTCGATATCTCCGTGGCGGCGTAT | | |
| | | | CCGGAAGTTCACCCGGAAGCAAAAAGCGCTCAGGCGGA | | |
| | | | TTTGCTTAATCTGAAACGCAAAGTGGATGCCGGAGCCA | | |
| | | | ACCGCGCGATTACTCAGTTCTTCTTCGATGTCGAAAGC | | |
| | | | TACCTGCGTTTTCGTGACCGCTGTGTATCGGCGGGCAT | | |
| | | | TGATGTGGAAATTATTCCGGGAATTTTGCCGGTATCTA | | |
| | | | ACTTTAAACAGGCGAAGAAATTTGCCGATATGACCAAC | | |
| | | | GTGCGTATTCCGGCGTGGATGGCGCAAATGTTCGACGG | | |
| | | | TCTGGATGATGATGCCGAAACCCGCAAACTGGTTGGCG | | |
| | | | CGAATATTGCCATGGATATGGTGAAGATTTTAAGCCGT | | |
| | | | GAAGGAGTGAAAGATTTCCACTTCTATACGCTTAACCG | | |
| | | | TGCTGAAATGAGTTACGCGATTTGCCATACGCTGGGGG | | |
| | | | TTCGACCTGGTTTA | | |
| cysE | Mycobacterium | AE007080 | ATGCTGACGGCCATGCGGGGCGACATCCGAGCAGCCCG | 156 | |
| | tuberculosis | | GGAGCGGGATCCGGCGGCCCCTACCGCGCTGGAAGTCA | | |
| | (use this to | | TCTTCTGCTACCCGGGCGTGCACGCCGTGTGGGGCCAC | | |
| | clone M. | | CGCCTCGCCCACTGGCTGTGGCAGCGTGGCGCCAGGCT | | |
| | smegmatis | | GCTCGCGCGGGCAGCTGCCGAATTCACTCGCATCCTGA | | |
| | gene) | | CCGGTGTAGATATCCACCCCGGTGCCGTCATCGGTGCT | | |
| | | | CGCGTGTTCATCGACCACGCGACCGGCGTGGTGATCGG | | |
| | | | AGAAACCGCGGAGGTCGGCGACGACGTCACGATCTATC | | |
| | | | ACGGCGTCACTCTCGGCGGCAGTGGCATGGTTGGCGGG | | |
| | | | AAACGCCATCCCACCGTCGGTGACCGCGTGATCATCGG | | |
| | | | CGCCGGGGCCAAGGTCCTCGGTCCGATCAAGATCGGCG | | |
| | | | AGGACAGCCGGATCGGCGCCAATGCCGTCGTGGTCAAG | | |
| | | | CCCGTCCCGCCGAGCGCGGTGGTGGTCGGGGTGCCCGG | | |
| | | | GCAGGTCATCGGCCAAAGCCAGCCCAGTCCCGGCGGCC | | |
| | | | CGTTTGATTGGAGGCTGCCCGATCTCGTGGGAGCCAGC | | |
| | | | CTCGATTCGCTGCTCACCAGGGTGGCCAGGCTGGACGC | | |
| | | | CCTCGGCGGCGGCCCGCAAGCAGCAGGAGTCATCCGGC | | |
| | | | CACCCGAAGCCGGGATATGGCACGGCGAGGACTTCTCG | | |
| | | | ATCTGA | | |
| cysE | Mycobacterium | Z98741 | ATGTTTGCGGCAATCCGGCGTGATATCCAGGCAGCAAG | 157 | |
| | leprae (use this | | ACAGCGAGATCCGGCACAGCCCACGGTGCTGGAGGTCA | | |
| | to clone M. | | TCTGCTGCTACCCAGGCGTGCACGCCGTCTGGGGTCAT | | |
| | smegmatis | | CGAATCAGTCACTGGTTGTGGAATCGTCGCGCCAGACT | | |
| | gene) | | GGCCGCGCGGGCGTTCGCCGAACTCACCCGCATCCTGA | | |
| | | | CTGGGGTCGACATCCACCCCGGTGCCGTGCTCGGAGCC | | |
| | | | GGCCTGTTCATCGATCACGCGACCGGCGTGGTGATCGG | | |
| | | | GGAAACCGCGGAAGTGGGCGATGACGTCACCATCTTCC | | |
| | | | ATGGAGTCACTCTCGGCGGCACCGGCCGGGAAACGGGT | | |
| | | | AAACGTCACCCAACCATCGGGGATCGAGTAACCATCGG | | |
| | | | CGCCGGCGCCAAGGTCCTCGGTGCCATCAAGATCGGCG | | |
| | | | AGGACAGCCGGATTGGCGCCAACGCAGTCGTGGTCAAG | | |
| | | | GAGGTCCCAGCCAGCGCTGTGGCCGTCGGGGTTCCCGG | | |
| | | | ACAAATCATCAGCAGCGACAGCCCGGCCAACGGGGACG | | |
| | | | ATTCTGTGCTGCCCGACTTCGTGGGCGTCAGCCTGCAA | | |
| | | | TCCCTGCTCACCAGGGTGGCCAAGCTGGAAGCCGAAGA | | |
| | | | CGGCGGTTCGCAAACCTACCGCGTCATCCGGCTACCCG | | |
| | | | AAGCCGGGGTTTGGCACGGCGAGGACTTCTCAATCTGA | | |
| cysE | Lactobacillus | AL935252 | GTGTTTCAGACGGCTCGTGCCATTCTCAATCGTGACCC | 158 | |
| | plantarum | | CGCCGCGATCAATTTGCGGACAGTTATGTTGACCTATC | | |
| | | | CTGGTATTCACGCGCTCGCCTGGTACCGGGTTGCCCAT | | |
| | | | TATTTTGAAACACACCGTTTACCATTATTGGCCGCCTT | | |
| | | | GCTGAGCCAACATGCGGCCCGGCATACCGGGATTCTGA | | |
| | | | TTCACCCGGCCGCGCAAATTGGTCACCGGGTCTTCTTT | | |
| | | | GACCATGGTATTGGTACTGTCATTGGTGCAACGGCGGT | | |
| | | | CATTGAAGACGACGTTACAATTTTACACGGCGTCACTT | | |
| | | | TAGGCGCACGTAAAACCGAACAAGCTGGGCGCCGGCAT | | |
| | | | CCCTATGTTTGTCGCGGTGCTTTCATTGGTGCCCACGC | | |
| | | | CCAACTCTTGGGCCCTATTACGATTGGCGCCAACAGTA | | |
| | | | AAATTGGTGCTGGTGCGATTGTTTTAGACAGCGTTCCC | | |
| | | | GCCCACGTTACTGCGGTCGGTAACCCGGCCCATCTAGT | | |
| | | | TGCCACTCAATTGCATGCTTATCATGAAGCAACCAGCA | | |
| | | | ATCAAGCTTGA | | |
| cysE | Corynebacterium | AX405283 | ATGCTCTCGACAATAAAAATGATCCGTGAAGATCTCGC | 268 | |
| | glutamicum | | AAACGCTCGTGAACACGATCCAGCAGCCCGAGGCGATT | | |
| | | | TAGAAAACGCAGTGGTTTACTCCGGACTCCACGCCATC | | |
| | | | TGGGCACATCGAGTTGCCAACAGCTGGTGGAAATCCGG | | |
| | | | TTTCCGCGGCCCCGCCCGCGTATTAGCCCAATTCACCC | | |
| | | | GATTCCTCACCGGCATTGAAATTCACCCCGGTGCCACC | | |
| | | | ATTGGTCGTCGCTTTTTTATTGACCACGGAATGGGAAT | | |
| | | | CGTCATCGGCGAAACCGCTGAAATCGGCGAAGGCGTCA | | |
| | | | TGCTCTACCACGGCGTCACCCTCGGCGGACAGGTTCTC | | |
| | | | ACCCAAACCAAGCGCCACCCCACGCTCTGCGACAACGT | | |
| | | | GACAGTCGGCGCGGGCGCAAAAATCTTAGGTCCCATCA | | |
| | | | CCATCGGCGAAGGCTCCGCAATTGGCGCCAATGCAGTT | | |
| | | | GTCACCAAAGACGTGCCGGCAGAACACATCGCAGTCGG | | |
| | | | AATTCCTGCGGTAGCACGCCCACGTGGCAAGACAGAGA | | |
| | | | AGATCAAGCTCGTCGATCCGGACTATTACATTTAA | | |
| cysE | Escherichia coli | NC_000913 | ATGTCGTGTGAAGAACTGGAAATTGTCTGGAACAATAT | 269 | |
| | | | TAAAGCCGAAGCCAGAACGCTGGCGGACTGTGAGCCAA | | |
| | | | TGCTGGCCAGTTTTTACCACGCGACGCTACTCAAGCAC | | |
| | | | GAAAACCTTGGCAGTGCACTGAGCTACATGCTGGCGAA | | |
| | | | CAAGCTGTCATCGCCAATTATGCCTGCTATTGCTATCC | | |
| | | | GTGAAGTGGTGGAAGAAGCCTACGCCGCTGACCCGGAA | | |
| | | | ATGATCGCCTCTGCGGCCTGTGATATTCAGGCGGTGCG | | |
| | | | TACCCGCGACCCGGCAGTCGATAAATACTCAACCCCGT | | |
| | | | TGTTATACCTGAAGGGTTTTCATGCCTTGCAGGCCTAT | | |
| | | | CGCATCGGTCACTGGTTGTGGAATCAGGGGCGTCGCGC | | |
| | | | ACTGGCAATCTTTCTGCAAAACCAGGTTTCTGTGACGT | | |
| | | | TCCAGGTCGATATTCACCCGGCAGCAAAAATTGGTCGC | | |
| | | | GGTATCATGCTTGACCACGCGACAGGCATCGTCGTTGG | | |
| | | | TGAAACGGCGGTGATTGAAAACGACGTATCGATTCTGC | | |
| | | | AATCTGTGACGCTTGGCGGTACGGGTAAATCTGGTGGT | | |
| | | | GACCGTCACCCGAAAATTCGTGAAGGTGTGATGATTGG | | |
| | | | CGCGGGCGCGAAAATCCTCGGCAATATTGAAGTTGGGC | | |
| | | | GCGGCGCGAAGATTGGCGCAGGTTCCGTGGTGCTGCAA | | |
| | | | CCGGTGCCGCCGCATACCACCGCCGCTGGCGTTCCGGC | | |
| | | | TCGTATTGTCGGTAAACCAGACAGCGATAAGCCATCAA | | |
| | | | TGGATATGGACCAGCATTTCAACGGTATTAACCATACA | | |
| | | | TTTGAGTATGGGGATGGGATC | | |
| serA | Mycobacterium | AL021287 | GTGAGCCTGCCTGTTGTGTTGATCGCCGACAAACTTGC | 159 | |
| | tuberculosis | | CCCATCAACGGTTGCCGCCTTGGGAGATCAGGTCGAGG | | |
| | (use this to | | TGCGCTGGGTTGACGGTCCGGACCGAGACAAGCTGCTG | | |
| | clone M. | | GCCGCGGTGCCCGAAGCGGACGCGCTGCTGGTGCGATC | | |
| | smegmatis | | GGCCACCACGGTTGACGCCGAGGTGCTGGCCGCCGCCC | | |
| | gene) | | CCAAGCTCAAGATCGTCGCGCGCGCCGGCGTCGGGCTG | | |
| | | | GACAACGTCGACGTGGACGCCGCGACGGCCCGCGGCGT | | |
| | | | GCTGGTGGTCAACGCCCCGACGTCGAACATCCACAGCG | | |
| | | | CCGCGGAGCATGCGCTGGCGCTGCTGCTGGCCGCCTCA | | |
| | | | CGCCAGATTCCGGCGGCCGACGCGTCGCTGCGCGAGCA | | |
| | | | CACCTGGAAGCGTTCGTCGTTTTCCGGTACCGAGATCT | | |
| | | | TCGGCAAAACCGTCGGCGTGGTGGGTCTGGGCCGCATC | | |
| | | | GGGCAGTTGGTCGCCCAGCGGATCGCTGCGTTCGGCGC | | |
| | | | TTACGTCGTCGCCTATGACCCGTACGTTTCGCCGGCCC | | |
| | | | GTGCGGCGCAGCTGGGCATCGAACTGCTGTCCCTGGAC | | |
| | | | GACCTGCTGGCCCGCGCCGATTTCATCTCGGTGCACCT | | |
| | | | ACCGAAAACACCGGAGACGGCGGGACTGATCGACAAGG | | |
| | | | AGGCGCTGGCGAAGACCAAGCCGGGCGTCATCATCGTC | | |
| | | | AACGCCGCGCGCGGCGGCCTGGTGGACGAGGCGGCACT | | |
| | | | GGCCGACGCGATCACCGGCGGCCACGTGCGGGCGGCCG | | |
| | | | GTCTGGACGTGTTCGCCACCGAACCGTGCACCGACAGC | | |
| | | | CCGCTGTTCGAGCTGGCACAGGTGGTGGTCACACCGCA | | |
| | | | TCTGGGTGCGTCCACCGCGGAGGCGCAGGACCGGGCGG | | |
| | | | GCACCGACGTCGCCGAGAGCGTGCGGCTGGCCCTGGCA | | |
| | | | GGGGAATTCGTGCCCGACGCGGTCAACGTCGGCGGCGG | | |
| | | | AGTGGTCAACGAGGAGGTGGCGCCCTGGCTGGATCTGG | | |
| | | | TGCGTAAGCTCGGCGTGCTGGCGGGTGTGTTGTCCGAC | | |
| | | | GAACTGCCGGTGTCGTTGTCGGTGCAGGTGCGCGGTGA | | |
| | | | GCTGGCCGCCGAAGAGGTTGAGGTGCTGCGCCTTTCGG | | |
| | | | CGCTGCGCGGCCTGTTCTCGGCGGTGATCGAGGATGCG | | |
| | | | GTGACATTTGTCAACGCACCGGCATTGGCCGCCGAACG | | |
| | | | TGGCGTCACCGCCGAGATCTGTAAGGCCTCGGAAAGCC | | |
| | | | CCAACCACCGCAGCGTCGTCGACGTTCGCGCGGTCGGC | | |
| | | | GCGGACGGTTCGGTGGTGACCGTCTCGGGCACGCTGTA | | |
| | | | TGGCCCACAGCTGTCGCAGAAGATCGTGCAGATCAACG | | |
| | | | GCCGCCACTTTGATCTGCGCGCCCAGGGGATCAACCTG | | |
| | | | ATCATCCACTACGTCGACCGGCCGGGAGCGCTGGGCAA | | |
| | | | GATCGGCACGTTGCTGGGGACGGCCGGGGTGAATATCC | | |
| | | | AGGCCGCGCAGCTCTCCGAAGACGCCGAAGGCCCGGGC | | |
| | | | GCGACGATTCTGCTGCGGCTGGACCAAGACGTGCCCGA | | |
| | | | CGACGTGCGGACGGCGATCGCGGCGGCGGTGGACGCCT | | |
| | | | ACAAGCTCGAGGTTGTCGATCTGTCGTGA | | |
| serA | Mycobacterium | Z99263 | GTGGACCTGCCTGTTGTGTTAATTGCCGACAAACTCGC | 160 | |
| | leprae (use this | | CCAATCAACCGTGGCTGCCCTGGGAGACCAAGTCGAGG | | |
| | to clone M. | | TGCGGTGGGTGGACGGTCCAGACCGGACGAAGCTGTTA | | |
| | smegmatis | | GCTGCAGTACCCGAGGCCGACGCGTTGTTGGTGCGGTC | | |
| | gene) | | GGCCACTACTGTCGACGCCGAGGTGCTGGCAGCCGCTC | | |
| | | | CTAAGCTCAAGATCGTCGCCCGTGCCGGGGTAGGGCTA | | |
| | | | GACAACGTTGATGTCGATGCCGCCACCGCGCGCGGTGT | | |
| | | | CCTGGTAGTCAACGCCCCAACGTCGAACATTCACAGCG | | |
| | | | CCGCTGAGCACGCGTTGGCGCTGCTATTGGCAGCTTCT | | |
| | | | CGGCAGATCGCGGAGGCCGACGCCTCACTGCGTGCACA | | |
| | | | CATCTGGAAACGGTCGTCGTTCTCCGGCACCGAAATTT | | |
| | | | TCGGCAAGACCGTCGGCGTGGTGGGGCTGGGTCGGATT | | |
| | | | GGGCAGTTGGTTGCCGCACGGATAGCAGCGTTCGGGGC | | |
| | | | TCACGTTATCGCTTACGACCCGTATGTGGCGCCGGCAC | | |
| | | | GGGCCGCGCAGCTTGGTATCGAGCTGATGTCTTTTGAC | | |
| | | | GATCTCCTAGCCCGGGCCGATTTTATCTCAGTGCATTT | | |
| | | | GCCGAAGACGCCCGAGACGGCGGGCCTGATCGACAAGG | | |
| | | | AGGCGCTGGCCAAAACCAAGCCCGGTGTCATCATTGTC | | |
| | | | AATGCCGCACGCGGCGGCTTAGTGGACGAGGTGGCGCT | | |
| | | | AGCCGATGCGGTGCGCAGCGGACATGTTCGGGCGGCCG | | |
| | | | GTCTAGATGTGTTTGCCACCGAACCGTGCACCGATAGC | | |
| | | | CCGCTGTTTGAACTATCGCAGGTGGTGGTGACACCGCA | | |
| | | | TCTGGGGGCGTCTACCGCCGAAGCCCAGGATCGAGCAG | | |
| | | | GTACTGATGTGGCCGAAAGCGTGCGGCTGGCGCTGGCG | | |
| | | | GGGGAGTTTGTGCCTGACGCGGTCAACGTGGACGGGGG | | |
| | | | CGTGGTCAACGAAGAGGTGGCTCCCTGGCTGGACTTGG | | |
| | | | TGTGCAAGCTTGGGGTGCTGGTAGCCGCGTTATCCGAT | | |
| | | | GAACTGCCGGCGTCGTTGTCGGTGCACGTGCGTGGCGA | | |
| | | | GTTGGCTTCTGAAGACGTTGAAATATTGCGGCTTTCGG | | |
| | | | CCCTACGTGGGCTTTTCTCGACGGTCATAGAGGATGCT | | |
| | | | GTGACGTTCGTCAACGCACCGGCACTGGCCGCCGAACG | | |
| | | | AGGTGTGTCCGCTGAAATCACTACGGGCTCGGAGAGCC | | |
| | | | CCAACCATCGCAGTGTGGTCGACGTGCGGGCGGTCGCC | | |
| | | | TCCGACGGCTCGGTGGTCAACATAGCCGGTACGTTGTC | | |
| | | | TGGGCCGCAACTGGTGCAGAAGATCGTGCAGGTCAATG | | |
| | | | GTCGTAACTTTGATTTGCGTGCGCAGGGCATGAACTTG | | |
| | | | GTGATCAGGTATGTCGACCAACCTGGCGCTCTGGGCAA | | |
| | | | GATTGGCACTTTGCTGGGCGCGGCCGGGGTGAATATCC | | |
| | | | AAGCTGCTCAGCTGTCTGAGGACACCGAGGGGCCAGGT | | |
| | | | GCGACGATTCTGTTGAGGCTGGATCAAGACGTGCCGGG | | |
| | | | TGATGTGCGGTCGGCGATCGTGGCAGCGGTGAGTGCCA | | |
| | | | ACAAGCTTGAGGTAGTCAATCTGTCATGA | | |
| serA | Thermobifida | NZ_AAAQ010 | GTGGCTGCGACCGCAGTCGAACCCACACGCACTCCCTC | 161 | |
| | fusca | 00025 | TAAGGAATTCGTTGTGCCCAAGCCAGTCGTCCTGGTCG | | |
| | | | CGGAAGAACTTTCGCCCGCAGGAATCGCGCTGTTGGAA | | |
| | | | GAGGACTTTGAAGTCCGCCACGTCAACGGCGCCGACCG | | |
| | | | TTCCCAGCTCCTTCCCGCGCTCGCCGGAGTCGACGCGC | | |
| | | | TGATCGTGCGCAGCGCCACCAAAGTGGACGCTGAGGTG | | |
| | | | CTGGCCGCGGCGCCCTCCCTCAAGGTTGTGGCGCGTGC | | |
| | | | GGGCGTCGGACTGGACAACGTGGATGTCGAGGCCGCCA | | |
| | | | CCAAGGCGGGCGTGCTCGTCGTCAACGCGCCCACCTCC | | |
| | | | AACATCATCAGTGCAGCGGAACAGGCCATCAACCTGCT | | |
| | | | CTTGGCCACGGCCCGCAACACTGCTGCTGCCCACGCGG | | |
| | | | CCCTCGTGCGCGGCGAGTGGAAGCGTTCCAAGTACACC | | |
| | | | GGCGTCGAACTGTACGACAAAACCGTCGGCATCGTGGG | | |
| | | | CCTGGGACGGATCGGCGTGCTCGTCGCCCAGCGGCTCC | | |
| | | | AGGCGTTCGGCACCAAGCTGATCGCCTACGACCCCTTC | | |
| | | | GTGCAGCCTGCCCGGGCCGCGCAGCTGGGGGTGGAGCT | | |
| | | | CGTCGAGCTCGACGAGCTGCTGGAGCGCAGCGACTTCA | | |
| | | | TCACGATCCACCTGCCCAAGACGAAGGACACGATCGGC | | |
| | | | CTGATCGGCGAGGAAGAGCTGCGCAAGGTCAAGCCGAC | | |
| | | | GGTCCGGATCATCAACGCTGCGCGCGGCGGGATCGTGG | | |
| | | | ACGAGACGGCCCTCTACCACGCGCTCAAGGAAGGTCGT | | |
| | | | GTGGCCGGCGCTGGGCTGGACGTGTTCGCCAAGGAGCC | | |
| | | | TTGCACGGACAGCCCGCTGTTCGAGCTGGAGAACGTGG | | |
| | | | TGGTGGCTCCGCACCTGGGGGCCAGCACGCACGAGGCG | | |
| | | | CAGGAGAAGGCCGGGACCCAGGTGGCCCGGTCCGTCAA | | |
| | | | GCTTGCGCTCGCCGGCGAGTTCGTGCCGGACGCGGTCA | | |
| | | | ACATCCAGGGCAAGGGCGTGGCCGAGGACATCAAGCCG | | |
| | | | GGGCTGCCGCTGACGGAGAAGCTCGGCCGTATCCTCGC | | |
| | | | CGCGCTCGCCGACGGTGCGATCACCCGGGTCGAGGTGG | | |
| | | | AGGTCCGGGGCGAGATCGTCGCCCACGACGTCAAGGTG | | |
| | | | ATCGAGCTGGCCGCGCTCAAGGGCCTCTTCACGGACAT | | |
| | | | CGTGGAAGAGGCTGTGACCTACGTGAACGCGCCTCTGG | | |
| | | | TAGCCAAGGAGCGCGGTATCGAGGTGAGCCTGACCACC | | |
| | | | GAGGAGGAGAGCCCCGACTGGCGCAACGTCATCACGGT | | |
| | | | GCGGGCCATCCTCTCCGACGGCCAGCGCGTGTCGGTCT | | |
| | | | CGGGCACGCTGACCGGGCCGCGCCAGTTGGAGAAGCTT | | |
| | | | GTCGAGGTCAACGGCTACACCATGGAGATCGCGCCCAG | | |
| | | | CGAGCACATGGCGTTCTTCTCCTACCACGACCGTCCCG | | |
| | | | GTGTGGTCGGCGTAGTCGGCCAACTGCTCGGACAGGCG | | |
| | | | CAGGTGAACATCGCCGGCATGCAGGTCAGCCGGGACAA | | |
| | | | GGAGGGCGGTGCGGCGCTGATCGCGCTGACCGTGGACT | | |
| | | | CGGCGATCCCCGACGAGACCCTCGAGACGATCTCCAAG | | |
| | | | GAGATCGGCGCCGAGATCAGCCGCGTGGACTTGGTTGA | | |
| | | | CTGA | | |
| serA | Streptomyces | AL939124 | GTGAGCTCGAAACCCGTCGTACTCATCGCTGAAGAGCT | 162 | |
| | coelicolor | | GTCGCCCGCGACCGTGGACGCACTCGGCCCCGACTTCG | | |
| | | | AGATCCGCCACTGCAACGGCGCGGACCGGGCCGAACTG | | |
| | | | CTCCCCGCCATCGCCGACGTGGACGCGATCCTGGTCCG | | |
| | | | CTCCGCGACCAAGGTCGACGCCGAGGCCGTGGCCGCCG | | |
| | | | CCAAGAAGCTCAAGGTCGTCGCGCGCGCCGGGGTCGGC | | |
| | | | CTGGACAACGTCGACGTCTCCGCCGCCACCAAGGCCGG | | |
| | | | CGTGATGGTGGTCAACGCCCCGACCTCCAACATCGTCA | | |
| | | | CCGCCGCCGAGCTGGCCTGCGGCCTGATCGTCGCCACC | | |
| | | | GCCCGCAACATCCCGCAGGCCAACGCCGCGCTGAAGAA | | |
| | | | CGGCGAGTGGAAGCGCAGCAAGTACACCGGCGTGGAGC | | |
| | | | TGGCCGAGAAGACCCTCGGCGTCGTCGGCCTCGGCCGC | | |
| | | | ATCGGCGCGCTCGTCGCGCAGCGCATGTCGGCCTTCGG | | |
| | | | CATGAAGGTCGTCGCCTACGACCCCTACGTGCAGCCCG | | |
| | | | CGCGGGCCGCGCAGATGGGCGTCAAGGTGCTGTCCCTG | | |
| | | | GACGAGCTGCTGGAGGTCTCCGACTTCATCACGGTCCA | | |
| | | | CCTGCCCAAGACCCCCGAGACCCTCGGCCTGATCGGCG | | |
| | | | ACGAGGCGCTGCGCAAGGTCAAGCCGAGCGTCCGCATC | | |
| | | | GTCAACGCCGCGCGCGGCGGCATCGTCGACGAGGAGGC | | |
| | | | GCTGTACTCGGCGCTCAAGGAGGGCCGCGTCGCCGGCG | | |
| | | | CCGGCCTCGACGTGTACGCCAAGGAGCCCTGCACCGAC | | |
| | | | TCGCCGCTGTTCGAGTTCGACCAGGTGGTCGCCACCCC | | |
| | | | GCACCTCGGCGCCTCCACCGACGAGGCCCAGGAGAAGG | | |
| | | | CCGGCATCGCCGTCGCCAAGTCGGTCCGCCTGGCCCTC | | |
| | | | GCCGGTGAGCTGGTCCCCGACGCGGTCAACGTCCAGGG | | |
| | | | CGGTGTCATCGCCGAGGACGTCAAGCCCGGTCTGCCGC | | |
| | | | TCGCCGAGCGCCTCGGCCGCATCTTCACCGCGCTCGCG | | |
| | | | GGTGAGGTCGCCGTCCGCCTCGACGTCGAGGTCTACGG | | |
| | | | CGAGATCACCCAGCACGACGTGAAGGTGCTGGAGCTGT | | |
| | | | CCGCCCTCAAGGGCGTCTTCGAGGACGTCGTCGACGAG | | |
| | | | ACGGTGTCGTACGTCAACGCCCCGCTGTTCGCCCAGGA | | |
| | | | GCGCGGCGTCGAGGTCCGGCTGACCACCAGCTCGGAGT | | |
| | | | CCCCGGAGCACCGCAACGTCGTCATCGTGCGCGGCACC | | |
| | | | CTCTCGGACGGCGAGGAGGTGTCGGTCTCCGGCACGCT | | |
| | | | GGCCGGCCCGAAGCACCTCCAGAAGATCGTCGCCATCG | | |
| | | | GCGAGTACGACGTGGACCTCGCCCTCGCCGACCACATG | | |
| | | | GTCGTCCTGCGCTACGAGGACCGTCCCGGCGTCGTCGG | | |
| | | | CACCGTCGGCCGGATCATCGGCGAGGCGGGTCTCAACA | | |
| | | | TCGCCGGCATGCAGGTCGCCCGCGCGACGGTCGGCGGC | | |
| | | | GAGGCGCTGGCCGTCCTCACCGTCGACGACACGGTGCC | | |
| | | | CTCCGGGGTTCTGGCGGAGGTCGCGGCGGAGATCGGCG | | |
| | | | CCACGTCCGCCCGGTCCGTCAACCTCGTCTGA | | |
| serA | Lactobacillus | AL935254 | ATGACAAAAGTCTTTATTGCTGGTCAGCTTCCAGCCCA | 163 | |
| | plantarum | | AGCTAATACGTTACTTTTACAAAGTCAGTTAGTCATTG | | |
| | | | ATACTTATACCGGCGATAACCTGATCAGTCACGCGGAA | | |
| | | | CTCATCCGTCGAGTCGCTGATGCCGACTTTTTGATTAT | | |
| | | | CCCACTCTCAACTCAAGTAGATCAAGATGTCTTAGACC | | |
| | | | ACGCCCCACACCTTAAACTGATTGCTAATTTTGGTGCT | | |
| | | | GGCACTAATAACATCGATATCGCGGCAGCAGCTAAGCG | | |
| | | | CCAGATTCCAGTCACGAACACGCCAAACGTTTCGGCGG | | |
| | | | TCGCAACCGCTGAATCAACGGTCGGTTTGATTATCAGC | | |
| | | | CTAGCGCATCGTATCGTGGAAGGCGATCACTTAATGCG | | |
| | | | AACTAGCGGCTTTAACGGTTGGGCGCCACTATTCTTTC | | |
| | | | TCGGCCACAACTTACAAGGCAAGACACTCGGCATCTTA | | |
| | | | GGCCTTGGCCAAATTGGTCAAGCCGTTGCCAAACGATT | | |
| | | | ACACGCCTTTGACATGCCCATCTTATACAGCCAACACC | | |
| | | | ACCGCCTACCGATTAGCCGTGAAACGCAACTTGGCGCA | | |
| | | | ACCTTTGTCTCCCAGGATGAACTTTTACAGCGTGCCGA | | |
| | | | CATCGTCACTTTACACCTGCCGCTTACCACACAAACAA | | |
| | | | CCCATCTAATCGATAACGCTGCTTTTAGCAAAATGAAG | | |
| | | | TCCACGGCGCTCCTCATCAACGCCGCACGGGGGCCAAT | | |
| | | | TGTCGACGAGCAAGCACTTGTGACGGCGCTGCAACAAC | | |
| | | | ATCAAATTGCTGGCGCTGCACTCGACGTCTACGAACAT | | |
| | | | GAACCGCAAGTCACACCTGGTTTGGCCACGATGAACAA | | |
| | | | CGTCATTTTGACACCTCATCTTGGCAACGCAACGGTCG | | |
| | | | AAGCTCGCGATGGCATGGCTACCATTGTCGCGGAGAAT | | |
| | | | GTGATTGCGATGGCCCAACATCAGCCAATCAAGTACGT | | |
| | | | GGTTAACGACGTAACACCAGCATAG | | |
| serA | Coryne- | AP005278 | GTGCGTTCTGCTACCACTGTCGATGCTGAAGTCATCGC | 270 | |
| | bacterium | | CGCTGCCCCTAACTTGAAGATCGTCGGTCGTGCCGGCG | | |
| | glutamicum | | TGGGCTTGGACAACGTTGACATCCCTGCTGCCACTGAA | | |
| | | | GCTGGCGTCATGGTTGCTAACGCACCGACCTCTAATAT | | |
| | | | TCACTCCGCTTGTGAGCACGCAATTTCTTTGCTGCTGT | | |
| | | | CTACTGCTCGCCAGATCCCTGCTGCTGATGCGACGCTG | | |
| | | | CGTGAGGGCGAGTGGAAGCGGTCTTCTTTCAACGGTGT | | |
| | | | GGAAATTTTCGGAAAAACTGTCGGTATCGTCGGTTTTG | | |
| | | | GCCACATTGGTCAGTTGTTTGCTCAGCGTCTTGCTGCG | | |
| | | | TTTGAGACCACCATTGTTGCTTACGATCCTTACGCTAA | | |
| | | | CCCTGCTCGTGCGGCTCAGCTGAACGTTGAGTTGGTTG | | |
| | | | AGTTGGATGAGCTGATGAGCCGTTCTGACTTTGTCACC | | |
| | | | ATTCACCTTCCTAAGACCAAGGAAACTGCTGGCATGTT | | |
| | | | TGATGCGCAGCTCCTTGCTAAGTCCAAGAAGGGCCAGA | | |
| | | | TCATCATCAACGCTGCTCGTGGTGGCCTTGTTGATGAG | | |
| | | | CAGGCTTTGGCTGATGCGATTGAGTCCGGTCACATTCG | | |
| | | | TGGCGCTGGTTTCGATGTGTACTCCACCGAGCCTTGCA | | |
| | | | CTGATTCTCCTTTGTTCAAGTTGCCTCAGGTTGTTGTG | | |
| | | | ACTCCTCACTTGGGTGCTTCTACTGAAGAGGCTCAGGA | | |
| | | | TCGTGCGGGTACTGACGTTGCTGATTCTGTGCTCAAGG | | |
| | | | CGCTGGCTGGCGAGTTCGTGGCGGATGCTGTGAACGTT | | |
| | | | TCCGGTGGTCGCGTGGGCGAAGAGGTTGCTGTGTGGAT | | |
| | | | GGATCTGGCTCGCAAGCTTGGTCTTCTTGCTGGCAAGC | | |
| | | | TTGTCGACGCCGCCCCAGTCTCCATTGAGGTTGAGGCT | | |
| | | | CGAGGCGAGCTTTCTTCCGAGCAGGTCGATGCACTTGG | | |
| | | | TTTGTCCGCTGTTCGTGGTTTGTTCTCCGGAATTATCG | | |
| | | | AAGAGTCCGTTACTTTCGTCAACGCTCCTCGCATTGCT | | |
| | | | GAAGAGCGTGGCCTGGACATCTCCGTGAAGACCAACTC | | |
| | | | TGAGTCTGTTACTCACCGTTCCGTCCTGCAGGTCAAGG | | |
| | | | TCATTACTGGCAGCGGCGCGAGCGCAACTGTTGTTGGT | | |
| | | | GCCCTGACTGGTCTTGAGCGCGTTGAGAAGATCACCCG | | |
| | | | CATCAATGGCCGTGGCCTGGATCTGCGCGCAGAGGGTC | | |
| | | | TGAACCTCTTCCTGCAGTACACTGACGCTCCTGGTGCA | | |
| | | | CTGGGTACCGTTGGTACCAAGCTGGGTGCTGCTGGCAT | | |
| | | | CAACATCGAGGCTGCTGCGTTGACTCAGGCTGAGAAGG | | |
| | | | GTGACGGCGCTGTCCTGATCCTGCGTGTTGAGTCCGCT | | |
| | | | GTCTCTGAAGAGCTGGAAGCTGAAATCAACGCTGAGTT | | |
| | | | GGGTGCTACTTCCTTCCAGGTTGATCTTGAC | | |
| serA | Escherichia coli | NC_000913 | ATGGCAAAGGTATCGCTGGAGAAAGACAAGATTAAGTT | 271 | |
| | | | TCTGCTGGTAGAAGGCGTGCACCAAAAGGCGCTGGAAA | | |
| | | | GCCTTCGTGCAGCTGGTTACACCAACATCGAATTTCAC | | |
| | | | AAAGGCGCGCTGGATGATGAACAATTAAAAGAATCCAT | | |
| | | | CCGCGATGCCCACTTCATCGGCCTGCGATCCCGTACCC | | |
| | | | ATCTGACTGAAGACGTGATCAACGCCGCAGAAAAACTG | | |
| | | | GTCGCTATTGGCTGTTTCTGTATCGGAACAAACCAGGT | | |
| | | | TGATCTGGATGCGGCGGCAAAGCGCGGGATCCCGGTAT | | |
| | | | TTAACGCACCGTTCTCAAATACGCGCTCTGTTGCGGAG | | |
| | | | CTGGTGATTGGCGAACTGCTGCTGCTATTGCGCGGCGT | | |
| | | | GCCGGAAGCCAATGCTAAAGCGCACCGTGGCGTGTGGA | | |
| | | | ACAAACTGGCGGCGGGTTCTTTTGAAGCGCGCGGCAAA | | |
| | | | AAGCTGGGTATCATCGGCTACGGTCATATTGGTACGCA | | |
| | | | ATTGGGCATTCTGGCTGAATCGCTGGGAATGTATGTTT | | |
| | | | ACTTTTATGATATTGAAAATAAACTGCCGCTGGGCAAC | | |
| | | | GCCACTCAGGTACAGCATCTTTCTGACCTGCTGAATAT | | |
| | | | GAGCGATGTGGTGAGTCTGCATGTACCAGAGAATCCGT | | |
| | | | CCACCAAAAATATGATGGGCGCGAAAGAAATTTCACTA | | |
| | | | ATGAAGCCCGGCTCGCTGCTGATTAATGCTTCGCGCGG | | |
| | | | TACTGTGGTGGATATTCCGGCGCTGTGTGATGCGCTGG | | |
| | | | CGAGCAAACATCTGGCGGGGGCGGCAATCGACGTATTC | | |
| | | | CCGACGGAACCGGCGACCAATAGCGATCCATTTACCTC | | |
| | | | TCCGCTGTGTGAATTCGACAACGTCCTTCTGACGCCAC | | |
| | | | ACATTGGCGGTTCGACTCAGGAAGCGCAGGAGAATATC | | |
| | | | GGCCTGGAAGTTGCGGGTAAATTGATCAAGTATTCTGA | | |
| | | | CAATGGCTCAACGCTCTCTGCGGTGAACTTCCcGGAAG | | |
| | | | TCTCGCTGCCACTGCACGGTGGGCGTCGTCTGATGCAC | | |
| | | | ATCCACGAAAACCGTCCGGGCGTGCTAACTGCGCTGAA | | |
| | | | CAAAATCTTCGCCGAGCAGGGCGTCAACATCGCCGCGC | | |
| | | | AATATCTGCAAACTTCCGCCCAGATGGGTTATGTGGTT | | |
| | | | ATTGATATTGAAGCCGACGAAGACGTTGCCGAAAAAGC | | |
| | | | GCTGCAGGCAATGAAAGCTATTCCGGGTACCATTCGCG | | |
| | | | CCCGTCTGCTGTAC | | |
| lysE | Mycobacterium | Z74025 | GTGAACTCACCACTGGTCGTCGGCTTCCTGGCCTGCTT | 164 | |
| | tuberculosis | | CACGCTGATCGCCGCGATTGGCGCGCAGAACGCATTCG | | |
| | (use this to | | TGCTGCGGCAGGGAATCCAGCGTGAGCACGTGCTGCCG | | |
| | clone M. | | GTGGTGGCGCTGTGCACGGTGTCCGACATCGTGCTGAT | | |
| | smegmatis | | CGCCGCCGGTATCGCGGGGTTCGGCGCATTGATCGGCG | | |
| | gene) | | CACATCCGCGTGCGCTCAATGTCGTCAAGTTTGGCGGC | | |
| | | | GCCGCCTTCCTAATCGGCTACGGGCTACTTGCGGCCCG | | |
| | | | GCGGGCGTGGCGACCTGTTGCGCTGATCCCATCTGGCG | | |
| | | | CCACGCCGGTTCGCTTAGCCGAGGTCCTGGTGACCTGT | | |
| | | | GCGGCATTCACGTTCCTCAACCCACACGTCTACCTCGA | | |
| | | | CACCGTCGTGTTGCTAGGCGCGCTGGCCAACGAGCACA | | |
| | | | GCGACCAGCGCTGGCTGTTCGGCCTCGGCGCGGTCACA | | |
| | | | GCCAGTGCGGTATGGTTCGCCACCCTCGGGTTCGGAGC | | |
| | | | CGGCCGGTTGCGCGGGCTGTTCACCAACCCCGGCTCGT | | |
| | | | GGAGAATCCTCGACGGCCTGATCGCGGTCATGATGGTT | | |
| | | | GCGCTGGGAATCTCGCTGACCGTGACCTAG | | |
| lysE | Mycobacterium | Z77162 | ATGATGACGCTCAAGGTCGCGATCGGCCCGCAAAACGC | 165 | |
| | tuberculosis | | ATTTGTCCTGCGCCAAGGAATTAGGCGAGAATACGTGC | | |
| | (use this to | | TGGTCATTGTGGCGCTGTGCGGGATCGCTGATGGGGCA | | |
| | clone M. | | CTGATTGCCGCGGGCGTTGGCGGCTTCGCTGCGCTGAT | | |
| | smegmatis | | TCACGCTCATCCCAATATGACTTTGGTTGCCCGATTTG | | |
| | gene) | | GCGGCGCAGCGTTCTTGATTGGCTACGCGCTATTGGCC | | |
| | | | GCGCGGAACGCGTGGCGCCCGAGCGGGCTGGTGCCGTC | | |
| | | | GGAATCGGGGCCGGCTGCGCTGATCGGCGTGGTGCAAA | | |
| | | | TGTGCCTGGTGGTGACCTTTCTCAACCCACACGTCTAT | | |
| | | | CTGGACACTGTGGTGTTGATCGGTGCCCTCGCCAATGA | | |
| | | | GGAATCAGATCTGCGGTGGTTTTTCGGAGCCGGTGCCT | | |
| | | | GGGCCGCCAGCGTCGTATGGTTCGCCGTGTTGGGATTT | | |
| | | | AGCGCGGGCCGGCTACAGCCATTCTTCGCAACTCCAGC | | |
| | | | TGCTTGGCGCATTCTTGATGCGCTGGTTGCCGTGACGA | | |
| | | | TGATTGGGGTCGCCGTCGTTGTGCTCGTCACGTCACCA | | |
| | | | AGTGTGCCGACGGCCAATGTCGCACTGATCATTTGA | | |
| lysE | Streptomyces | AL939131 | ATGAACAACGCCCTCACGGCGGCCGCCGCCGGTTTCGG | 166 | |
| | coelicolor | | CACCGGCCTCTCGCTCATCGTCGCCATCGGCGCCCAGA | | |
| | | | ACGCCTTCGTCCTGCGGCAGGGGGTCCGCCGTGACGCG | | |
| | | | GTGCTCGCCGTGGTCGGCATCTGCGCGCTGTCCGACGC | | |
| | | | CGTGCTCATCGCCCTGGGCGTCGGCGGGGTCGGCGCCG | | |
| | | | TGGTGGTGGCGTGGCCGGGCGCGCTGACCGCCGTCGGC | | |
| | | | TGGATCGGCGGCGCGTTCCTGCTCTGCTACGGAGCCCT | | |
| | | | GGCGGCCCGGCGGGTGTTCCGGCCGTCCGGGGCGCTGC | | |
| | | | GGGCGGACGGCGCCGCCGCGGGCTCGCGCCGCCGGGCC | | |
| | | | GTGCTCACCTGCCTGGCGCTGACCTGGCTCAACCCGCA | | |
| | | | CGTCTACCTCGACACCGTGTTCCTGCTGGGCTCCGTCG | | |
| | | | CCGCCGACCGGGGGCCGCTGCGCTGGACCTTCGGCCTC | | |
| | | | GGAGCCGCCGCCGCCAGCCTGGTCTGGTTCGCCGCGCT | | |
| | | | CGGCTTCGGCGCCCGCTACCTCGGCCGCTTCCTGTCCC | | |
| | | | GGCCCGTCGCCTGGCGGGTCCTCGACGGACTGGTGGCC | | |
| | | | GCCACCATGATCGTCCTCGGCGTCTCCCTCGTCGCCGG | | |
| | | | GGCCTGA | | |
| lysE | Lactobacillus | AL935256 | ATGCAAGTGTTTTTACAAGGATTATTATTTGGAATTGT | 167 | |
| | plantarum | | TTACATTGCACCAATCGGGATGCAAAACTTATTTGTGG | | |
| | | | TTTCGACAGCTATTGAACAACCATTGCAACGGGCATTG | | |
| | | | CGGGTGGCTTTAATTGTAATTGCGTTCGATACGTCGCT | | |
| | | | CTCCCTGGCTTGCTTTTATGGGGTGGGCCGATTGTTGC | | |
| | | | AGACCACTCCCTGGCTCGAATTAGGGGTGTTGTTGATT | | |
| | | | GGGAGTTTATTGGTCTTTTACATTGGCTGGAATCTGTT | | |
| | | | GCGGAAAAAGGCCACGGCAATGGGGACCCTCGACGCGG | | |
| | | | ACTTTTCATATAAAGCAGCGATTCTGACAGCTTTTTCG | | |
| | | | GTAGCATGGCTGAATCCGCAAGCACTGATTGATGGTTC | | |
| | | | CGTGTTGTTGGCGGCGTTTCGGGTGTCAATCCCGGCGG | | |
| | | | CACTGACCCATTTCTTTATGTTGGGGGTCATCCTAGCA | | |
| | | | TCCATTATTTGGTTCATCGGTCTGACCAGCTTGATCAG | | |
| | | | TAAGTTTAAACATCTCATGCAACCACGAGTCCTACTCT | | |
| | | | GGATCAATCGAATCTGTGGTGGCATCATTATTCTATAC | | |
| | | | GGCGTGCAGTTGCTAGCAACCTTCATCACGAAAATATAG | | |
| lysE | Coryne- | X96471 | ATGGAAATCTTCATTACAGGTCTGCTTTTGGGGGCCAG | 272 | |
| | bacterium | | TCTTTTACTGTCCATCGGACCGCAGAATGTACTGGTGA | | |
| | glutamicum | | TTAAACAAGGAATTAAGCGCGAAGGACTCATTGCGGTT | | |
| | | | CTTCTCGTGTGTTTAATTTCTGACGTCTTTTTGTTCAT | | |
| | | | CGCCGGCACCTTGGGCGTTGATCTTTTGTCCAATGCCG | | |
| | | | CGCCGATCGTGCTCGATATTATGCGCTGGGGTGGCATC | | |
| | | | GCTTACCTGTTATGGTTTGCCGTCATGGCAGCGAAAGA | | |
| | | | CGCCATGACAAACAAGGTGGAAGCGCCACAGATCATTG | | |
| | | | AAGAAACAGAACCAACCGTGCCCGATGACACGCCTTTG | | |
| | | | GGCGGTTCGGCGGTGGCCACTGACACGCGCAACCGGGT | | |
| | | | GCGGGTGGAGGTGAGCGTCGATAAGCAGCGGGTTTGGG | | |
| | | | TAAAGCCCATGTTGATGGCAATCGTGCTGACCTGGTTG | | |
| | | | AACCCGAATGCGTATTTGGACGCGTTTGTGTTTATCGG | | |
| | | | CGGCGTCGGCGCGCAATACGGCGACACCGGACGGTGGA | | |
| | | | TTTTCGCCGCTGGCGCGTTCGCGGCAAGCCTGATCTGG | | |
| | | | TTCCCGCTGGTGGGTTTCGGCGCAGCAGCATTGTCACG | | |
| | | | CCCGCTGTCCAGCCCCAAGGTGTGGCGCTGGATCAACG | | |
| | | | TCGTCGTGGCAGTTGTGATGACCGCATTGGCCATCAAA | | |
| | | | CTGATGTTGATGGGTTAG | | |
| metB | Mycobacterium | AL021897 | ATGAGCGAAGACCGCACGGGACACCAGGGAATCAGCGG | 168 | |
| | tuberculosis | | ACCGGCCACCCGCGCCATCCACGCTGGCTACCGCCCGG | | |
| | (use this to | | ATCCGGCGACCGGGGCGGTGAACGTGCCGATCTACGCC | | |
| | clone M. | | AGCAGCACCTTCGCCCAAGACGGCGTCGGCGGTCTGCG | | |
| | smegmatis | | TGGCGGTTTCGAATACGCACGCACCGGCAACCCCACCC | | |
| | gene) | | GGGCCGCATTGGAGGCCTCGCTGGCGGCAGTCGAGGAG | | |
| | | | GGTGCTTTCGCGCGGGCATTCAGTTCCGGGATGGCCGC | | |
| | | | GACCGACTGCGCCCTGCGGGCGATGTTACGGCCCGGAG | | |
| | | | ACCACGTCGTCATTCCCGATGACGCCTACGGCGGCACA | | |
| | | | TTCCGGTTGATAGACAAGGTGTTCACCCGGTGGGATGT | | |
| | | | CCAGTACACGCCGGTGCGGCTTGCCGATCTGGATGCGG | | |
| | | | TGGGTGCCGCGATTACTCCGCGCACCCGGCTGATTTGG | | |
| | | | GTGGAGACGCCCACCAATCCGCTACTGTCGATCGCCGA | | |
| | | | TATCACGGCCATTGCCGAGCTGGGCACAGACAGATCGG | | |
| | | | CAAAAGTATTGGTGGACAATACCTTTGCCTCACCCGCG | | |
| | | | TTGCAGCAGCCGTTGCGGCTGGGCGCCGATGTGGTGTT | | |
| | | | GCACTCGACTACCAAGTACATCGGCGGCCATTCCGACG | | |
| | | | TGGTGGGAGGTGCGCTGGTCACCAACGACGAAGAGCTG | | |
| | | | GACGAGGAGTTCGCTTTCTTGCAGAACGGCGCCGGCGC | | |
| | | | GGTGCCCGGACCATTCGACGCCTACCTGACCATGCGCG | | |
| | | | GCCTGAAGACCTTGGTGCTGCGGATGCAGCGGCACAGT | | |
| | | | GAAAATGCCTGTGCGGTAGCGGAATTCCTCGCTGATCA | | |
| | | | TCCGTCGGTGAGTTCTGTGTTGTATCCGGGTTTGCCCA | | |
| | | | GTCATCCCGGGCATGAGATTGCCGCGCGACAGATGCGC | | |
| | | | GGCTTCGGCGGCATGGTTTCGGTGCGGATGCGGGCCGG | | |
| | | | TCGGCGTGCGGCGCAGGACCTGTGTGCCAAGACCCGCG | | |
| | | | TCTTCATCCTGGCCGAGTCGCTGGGTGGGGTGGAGTCG | | |
| | | | CTGATCGAACATCCCAGCGCCATGACCCATGCGTCGAC | | |
| | | | GGCCGGTTCGCAATTGGAGGTGCCCGACGATCTGGTGC | | |
| | | | GGCTTTCGGTCGGTATCGAAGACATTGCCGACCTGCTC | | |
| | | | GGCGATCTCGAACAGGCCCTGGGTTAA | | |
| metB | Mycobacterium | U15183 | ATGAGCGAAGATTACCGGGGACACCACGGCATTACCGG | 169 | |
| | leprae (use this | | ACTAGCCACCAAAGCCATCCATGCTGGCTATCGTCCGG | | |
| | to clone M. | | ATCCGGCAACAGGGGCAGTGAATGTCCCGATTTATGCC | | |
| | smegmatis | | AGTAGTACTTTTGCCCAAGATGGCGTCGGTGAGTTGCG | | |
| | gene) | | TGGCGGATTCGAATACGCGCGTACCGGCAACCCCATGC | | |
| | | | GCGCCGCTTTAGAGGCATCCTTGGCCACGGTCGAAGAG | | |
| | | | GGCGTTTTTGCGCGAGCCTTCAGTTCCGGAATGGCTGC | | |
| | | | TAGCGACTGTGCCTTGCGGGTCATGCTGCGGCCGGGGG | | |
| | | | ACCACGTGATCATCCCGGATGACGTCTACGGCGGCACC | | |
| | | | TTCCGGCTGATAGACAAGGTCTTTACTCAATGGAACGT | | |
| | | | TGACTACACGCCGGTACCGCTGTCTGATTTGGACGCGG | | |
| | | | TCCGCGCCGCGATCACATCACGGACCCGGCTGATATGG | | |
| | | | GTGGAAACACCGACCAATCCGCTGCTGTCCATCGCAGA | | |
| | | | TATCACCAGCATCGGCGAACTAGGCAAAAAGCACTCAG | | |
| | | | TAAAGGTGTTGGTGGACAACACCTTTGCTTCACCCGCG | | |
| | | | CTGCAACAGCCGCTGATGCTGGGGGCAGACGTCGTGTT | | |
| | | | GCACTCGACCACAAAGTACATCGGCGGCCACTCTGATG | | |
| | | | TGGTGGGCGGCGCGCTAGTCACCAACGACGAAGAGCTG | | |
| | | | GACCAGGCTTTCGGCTTCTTGCAGAACGGAGCCGGTGC | | |
| | | | GGTGCCGAGCCCGTTCGACGCGTACCTAACGATGCGCG | | |
| | | | GATTGAAGACTTTAGTGCTGCGGATGCAGCGGCACAAC | | |
| | | | GAAAATGCCATTACTGTAGCGGAATTCCTGGCTGGGCA | | |
| | | | TCCGTCGGTGAGCGCCGTGCTGTATCCGGGCTTGCCCA | | |
| | | | GCCATCCCGGGCATGAGGTCGCTGCACGGCAGATGCGC | | |
| | | | GGCTTCGGCGGCATGGTTTCGTTGCGGATGCGAGCCGG | | |
| | | | CCGACTAGCCGCCCAGGATCTGTGTGCCCGCACCAAGG | | |
| | | | TGTTTACCTTGGCTGAATCCTTGGGTGGAGTGGAGTCG | | |
| | | | CTGATTGAGCAGCCCAGTGCCATGACGCACGCGTCGAC | | |
| | | | AACCGGGTCGCAATTGGAAGTACCCGACGACCTGGTGC | | |
| | | | GGCTTTCGGTCGGTATTGAAGACGTCGGCGACCTGCTG | | |
| | | | TGCGACCTCAAGCAGGCGTTAAACTAA | | |
| metB | Streptomyces | AL939122 | GTGCCCATGAGCGACAGGCACATCAGTCAGCACTTCGA | 170 | |
| | coelicolor | | GACGCTCGCGATCCACGCGGGCAACACCGCCGATCCCC | | |
| | | | TGACGGGCGCGGTCGTCCCGCCGATCTATCAGGTGTCG | | |
| | | | ACCTACAAGCAGGACGGCGTCGGCGGATTGCGCGGCGG | | |
| | | | CTACGAGTACAGCCGCAGCGCCAACCCGACCCGTACCG | | |
| | | | CGCTGGAGGAGAACCTCGCCGCCCTGGAGGGCGGCCGC | | |
| | | | CGCGGCCTCGCGTTCGCGTCCGGACTGGCGGCCGAGGA | | |
| | | | CTGCCTGTTGCGTACGCTGCTGCGCCCCGGCGACCACG | | |
| | | | TGGTGATCCCGAACGACGCGTACGGCGGCACCTTCCGC | | |
| | | | CTCTTCGCCAAGGTCGCCACCCGGTGGGGTGTGGAGTG | | |
| | | | GTCCGTGGCCGACACGAGCGACGCCGCCGCCGTGCGGG | | |
| | | | CCGCCCTCACCCCGAAGACCAAGGCGGTGTGGGTGGAG | | |
| | | | ACGCCCTCCAACCCGCTGCTCGGCATCACCGACATCGC | | |
| | | | GCAGGTCGCCCAGGTCGCCCGGGACGCCGGCGCCCGGC | | |
| | | | TCGTCGTCGACAACACCTTCGCCACCCCGTACCTCCAG | | |
| | | | CAGCCGCTGGCCCTCGGCGCCGACGTCGTCGTGCACTC | | |
| | | | GCTGACCAAGTACATGGGCGGGCACTCGGACGTCGTGG | | |
| | | | GCGGCGCGCTGATCGTGGGCGACCAGGAGCTGGGCGAG | | |
| | | | GAGCTGGCGTTCCACCAGAACGCGATGGGCGCGGTCGC | | |
| | | | CGGACCCTTCGACTCCTGGCTGGTGCTGCGCGGCACCA | | |
| | | | AGACCCTCGCCGTGCGCATGGACCGGCACAGCGAGAAC | | |
| | | | GCGACCAAGGTCGCCGACATGCTCTCCCGGCACGCGCG | | |
| | | | CGTGACGAGCGTGCTGTACCCGGGGCTGCCCGAGCACC | | |
| | | | CGGGGCACGAGGTCGCCGCCAAGCAGATGAAGGCGTTC | | |
| | | | GGCGGCATGGTGTCGTTCCGCGTCGAGGGCGGCGAGCA | | |
| | | | GGCCGCCGTCGAGGTGTGCAACCGCGCGAAGGTCTTCA | | |
| | | | CGCTCGGCGAGTCCCTCGGCGGCGTCGAGTCGCTGATC | | |
| | | | GAGCACCCGGGCCGGATGACGCACGCCTCCGCGGCGGG | | |
| | | | CTCGGCCCTGGAGGTGCCCGCCGACCTGGTGCGGCTGT | | |
| | | | CGGTCGGCATCGAGAACGCCGACGACCTGCTGGCCGAC | | |
| | | | CTCCAGCAGGCGCTGGGCTAG | | |
| metB | Thermobifida | NZ_AAAQ010 | ATGAGTTACGAGGGGTTTGAGACACTGGCCATCCACGC | 171 | |
| | fusca | 00041 | CGGTCAGGAGGCAGACGCCGAGACCGGGGCCGTGGTGG | | |
| | | | TCCCCATCTACCAGACGAGCACCTACCGCCAAGACGGG | | |
| | | | GTGGGCGGGCTGCGCGGCGGCTACGAGTACTCCCGCAC | | |
| | | | CGCCAACCCGACCCGCACGGCACTGGAAGAATGCCTGG | | |
| | | | CCGCGCTGGAAGGCGGGGTGCGGGGCCTGGCGTTCGCT | | |
| | | | TCCGGCATGGCCGCAGAGGACACCCTGCTCCGCACCAT | | |
| | | | CGCCCGACCCGGCGACCACCTCATCATCCCCAACGACG | | |
| | | | CCTACGGCGGCACGTTCCGCCTCGTCTCCAAGGTCTTC | | |
| | | | GAACGGTGGGGAGTGAGCTGGGACGCCGTCGACCTGTC | | |
| | | | CAACCCGGAGGCGGTGCGGACCGCAATCCGCCCGGAAA | | |
| | | | CCGTGGCGATCTGGGTGGAAACCCCCACCAACCCGCTG | | |
| | | | CTCAACATTGCGGACATCGCCGCGCTCGCGGACATCGC | | |
| | | | GCACGCCGCTGACGCGCTGCTGGTGGTCGACAACACCT | | |
| | | | TCGCCTCCCCGTACCTGCAGCGGCCGCTCAGCCTCGGT | | |
| | | | GCGGACGTGGTCGTGCACTCCACCACCAAATACCTGGG | | |
| | | | CGGCCACTCCGACGTGGTCGGCGGCGCCCTCGTGGTCG | | |
| | | | CCGACGCGGAACTGGGAGAGCGCCTCGCCTTCCACCAG | | |
| | | | AACTCGATGGGCGCGGTCGCGGGACCGTTCGACGCCTG | | |
| | | | GCTGACCCTGCGCGGCATCAAAACCCTCGGCGTGCGCA | | |
| | | | TGGACCGGCACTGCGCCAACGCGGAACGCGTCGTGGAA | | |
| | | | GCGCTCGTCGGCCACCCGGAAGTCGCCGAAGTGCTCTA | | |
| | | | CCCGGGCCTGTCCGACCACCCCGGCCACAAGGTGGCGG | | |
| | | | TCGACCAGATGCGCGCCTTCGGTGGCATGGTGTCGTTC | | |
| | | | CGCATGCGCGGCGGGGAGGAAGCCGCGTTGCGGGTGTG | | |
| | | | CGCGAAAACGAAAGTGTTCACCCTCGCTGAATCCTTGG | | |
| | | | GCGGGGTGGAGTCGCTGATCGAACACCCGGGGAAGATG | | |
| | | | ACCCACGCCTCCACCGCGGGCTCCCTCCTGGAAGTGCC | | |
| | | | CAGCGACCTGGTCCGGCTCTCCGTGGGTATCGAAACCG | | |
| | | | TCGACGACCTCGTCAACGACCTGCTCCAAGCATTGGAG | | |
| | | | CCGTAG | | |
| metB | Lactobacillus | AL935252 | ATGAAATTTGAAACCCAATTAATTCACGGTGGTATCAG | 172 | |
| | plantarum | | TGAGGATGCCACTACTGGCGCGACTTCGGTACCCATCT | | |
| | | | ACATGGCCTCGACCTTCCGCCAAACAAAAATCGGTCAA | | |
| | | | AATCAATACGAATATTCACGGACGGGAAATCCAACCCG | | |
| | | | GGCCGCCGTCGAAGCATTAATTGCCACCCTCGAACATG | | |
| | | | GCAGCGCTGGCTTCGCATTTGCTTCTGGCTCCGCTGCC | | |
| | | | ATTAATACCGTCTTCTCACTATTCTCGGCTGGTGATCA | | |
| | | | CATTATTGTGGGAAATGATGTCTACGGTGGCACCTTCC | | |
| | | | GCTTGATCGACGCCGTTTTGAAACACTTTGGCATGACT | | |
| | | | TTTACAGCCGTAGATACGCGTGACTTGGCCGCCGTTGA | | |
| | | | AGCCGCAATTACCCCCACAACTAAGGCGATTTATTTGG | | |
| | | | AAACACCGACGAACCCGTTATTACACATTACGGATATT | | |
| | | | GCTGCCATTGCGAAGCTCGCGCAAGCACACGATTTACT | | |
| | | | GAGTATCATCGACAACACCTTCGCCTCCCCATACGTCC | | |
| | | | AGAAGCCCCTGGATTTAGGCGTTGACATTGTTTTACAC | | |
| | | | AGTGCTTCCAAGTATCTCGGTGGTCACAGTGATGTTAT | | |
| | | | CGGTGGCTTGGTTGTCACCAAGACGCCAGCACTTGGCG | | |
| | | | AAAAAATCGGCTACTTGCAAAATGCCATCGGTAGTATT | | |
| | | | TTGGCCCCGCAAGAAAGCTGGCTATTACAACGTGGTAT | | |
| | | | GAAGACTCTGGCATTGCGCATGCAAGCCCACCTG~TA | | |
| | | | ATGCCGCTAAAATCTTTACTTACTTAAAGTCTCACCCA | | |
| | | | GCAGTTACTAAGATTTACTATCCAGGCGATCCTGATAA | | |
| | | | TCCCGATTTTTCGATTGCCAAGCAACAGATGAATGGCT | | |
| | | | TCGGCGCAATGATCTCGTTTGAATTACAACCAGGAATG | | |
| | | | AACCCCCAGACCTTCGTTGAACATTTACAAGTCATCAC | | |
| | | | GCTCGCCGAAAGTCTCGGAGCATTGGAAAGTTTAATTG | | |
| | | | AAATTCCAGCCTTAATGACTCACGGTGCCATCCCACGC | | |
| | | | ACAATTCGGCTACAGAATGGCATCAAAGACGAGCTGAT | | |
| | | | TCGCTTATCAGTCGGTGTTGAAGCCAGTGACGATTTGT | | |
| | | | TAGCAGACCTTGAGCGCGGGTTCGCTAGCATTCAGGCA | | |
| | | | GATTAA | | |
| metB | Coryne- | AF126953 | TTGTCTTTTGACCCAAACACCCAGGGTTTCTCCACTGC | 273 | |
| | bacterium | | ATCGATTCACGCTGGGTATGAGCCAGACGACTACTACG | | |
| | glutamicum | | GTTCGATTAACACCCCAATCTATGCCTCCACCACCTTC | | |
| | | | GCGCAGAACGCTCCAAACGAACTGCGCAAAGGCTACGA | | |
| | | | GTACACCCGTGTGGGCAACCCCACCATCGTGGCATTAG | | |
| | | | AGCAGACCGTCGCAGCACTCGAAGGCGCAAAGTATGGC | | |
| | | | CGCGCATTCTCCTCCGGCATGGCTGCAACCGACATCCT | | |
| | | | GTTCCGCATCATCCTCAAGCCGGGCGATCACATCGTCC | | |
| | | | TCGGCAACGATGCTTACGGCGGAACCTACCGCCTGATC | | |
| | | | GACACCGTATTCACCGCATGGGGCGTCGAATACACCGT | | |
| | | | TGTTGATACCTCCGTCGTGGAAGAGGTCAAGGCAGCGA | | |
| | | | TCAAGGACAACACCAAGCTGATCTGGGTGGAAACCCCA | | |
| | | | ACCAACCCAGCACTTGGCATCACCGACATCGAAGCAGT | | |
| | | | AGCAAAGCTCACCGAAGGCACCAACGCCAAGCTGGTTG | | |
| | | | TTGACAACACCTTCGCATCCCCATACCTGCAGCAGCCA | | |
| | | | CTAAAACTCGGCGCACACGCAGTCCTGCACTCCACCAC | | |
| | | | CAAGTACATCGGAGGACACTCCGACGTTGTTGGCGGCC | | |
| | | | TTGTGGTTACCAACGACCAGGAAATGGACGAAGAACTG | | |
| | | | CTGTTCATGCAGGGCGGCATCGGACCGATCCCATCAGT | | |
| | | | TTTCGATGCATACCTGACCGCCCGTGGCCTCAAGACCC | | |
| | | | TTGCAGTGCGCATGGATCGCCACTGCGACAACGCAGAA | | |
| | | | AAGATCGCGGAATTCCTGGACTCCCGCCCAGAGGTCTC | | |
| | | | CACCGTGCTCTACCCAGGTCTGAAGAACCACCCAGGCC | | |
| | | | ACGAAGTCGCAGCGAAGCAGATGAAGCGCTTCGGCGGC | | |
| | | | ATGATCTCCGTCCGTTTCGCAGGCGGCGAAGAAGCAGC | | |
| | | | TAAGAAGTTCTGTACCTCCACCAAACTGATCTGTCTGG | | |
| | | | CCGAGTCCCTCGGTGGCGTGGAATCCCTCCTGGAGCAC | | |
| | | | CCAGCAACCATGACCCACCAGTCAGCTGCCGGCTCTCA | | |
| | | | GCTCGAGGTTCCCCGCGACCTCGTGCGCATCTCCATTG | | |
| | | | GTATTGAAGACATTGAAGACCTGCTCGCAGATGTCGAG | | |
| | | | CAGGCCCTCAATAACCTTTAG | | |
| metB | Escherichia coli | NC_000913 | ATGACGCGTAAACAGGCCACCATCGCAGTGCGTAGCGG | 274 | |
| | | | GTTAAATGACGACGAACAGTATGGTTGCGTTGTCCCAC | | |
| | | | CGATCCATCTTTCCAGCACCTATAACTTTACCGGATTT | | |
| | | | AATGAACCGCGCGCGCATGATTACTCGCGTCGCGGCAA | | |
| | | | CCCAACGCGCGATGTGGTTCAGCGTGCGCTGGCAGAAC | | |
| | | | TGGAAGGTGGTGCTGGTGCAGTACTTACTAATACCGGC | | |
| | | | ATGTCCGCGATTCACCTGGTAACGACCGTCTTTTTGAA | | |
| | | | ACCTGGCGATCTGCTGGTTGCGCCGCACGACTGCTACG | | |
| | | | GCGGTAGCTATCGCCTGTTCGACAGTCTGGCGAAACGC | | |
| | | | GGTTGCTATCGCGTGTTGTTTGTTGATCAAGGCGATGA | | |
| | | | ACAGGCATTACGGGCAGCGCTGGCAGAAAAACCCAAAC | | |
| | | | TGGTACTGGTAGAAAGCCCAAGTAATCCATTGTTACGC | | |
| | | | GTCGTGGATATTGCGAAAATCTGCCATCTGGCAAGGGA | | |
| | | | AGTCGGGGCGGTGAGCGTGGTGGATAACACCTTCTTAA | | |
| | | | GCCCGGCATTACAAAATCCGCTGGCATTAGGTGCCGAT | | |
| | | | CTGGTGTTGCATTCATGCACGAAATATCTGAACGGTCA | | |
| | | | CTCAGACGTAGTGGCCGGCGTGGTGATTGCTAAAGACC | | |
| | | | CGGACGTTGTCACTGAACTGGCCTGGTGGGCAAACAAT | | |
| | | | ATTGGCGTGACGGGCGGCGCGTTTGACAGCTATCTGCT | | |
| | | | GCTACGTGGGTTGCGAACGCTGGTGCCGCGTATGGAGC | | |
| | | | TGGCGCAGCGCAACGCGCAGGCGATTGTGAAATACCTG | | |
| | | | CAAACCCAGCCGTTGGTGAAAAAACTGTATCACCCGTC | | |
| | | | GTTGCCGGAAAATCAGGGGCATGAAATTGCCGCGCGCC | | |
| | | | AGCAAAAAGGCTTTGGCGCAATGTTGAGTTTTGAACTG | | |
| | | | GATGGCGATGAGCAGACGCTGCGTCGTTTCCTGGGCGG | | |
| | | | GCTGTCGTTGTTTACGCTGGCGGAATCATTAGGGGGAG | | |
| | | | TGGAAAGTTTAATCTCTCACGCCGCAACCATGACACAT | | |
| | | | GCAGGCATGGCACCAGAAGCGCGTGCTGCCGCCGGGAT | | |
| | | | CTCCGAGACGCTGCTGCGTATCTCCACCGGTATTGAAG | | |
| | | | ATGGCGAAGATTTAATTGCCGACCTGGAAAATGGCTTC | | |
| | | | CGGGCTGCAAACAAGGGG | | |
| putative | Streptomyces | AL939116 | ATGGCCGGCATCGGGGCCTTCTGGTCGGTGTCCTTCCT | 173 | |
| threonine | coelicolor | | GCTGGTGCTGGTCCCGGGCGCGGACTGGGCCTACGCGA | | |
| efflux protein | | | TCACGGCGGGACTGCGCCACCGGTCGGTGCTGCCCGCC | | |
| 1 | | | GTCGGCGGCATGCTGAGCGGATACGTCCTGCTGACCGC | | |
| | | | CGTGGTCGCCGCGGGCCTGGCGACCGCGGTCGCCGGTT | | |
| | | | CACCGACGGTGCTGACCGCGCTGACGGCCGCCGGTGCG | | |
| | | | GCCTATCTGATCTGGCTAGGCGCCACGACCCTGGCCCG | | |
| | | | CCCCGCGGCGCCCCGGGCCGAGGAGGGCGACCAGGGAG | | |
| | | | ACGGCTCCGGCTCGTTGGTGGGCCGTGCGGCCAGAGGG | | |
| | | | GCGGGCATCAGCGGCCTCAACCCCAAGGCGCTGCTGCT | | |
| | | | GTTCCTCGCCCTGCTGCCGCAGTTCGCCGCCCGGGACG | | |
| | | | CGGACTGGCCCTTTGCCGCGCAGATCGTCGCCCTCGGC | | |
| | | | CTGGTGCACACGGCCAACTGCGCCGTGGTCTACACGGG | | |
| | | | CGTCGGCGCCACGGCACGCCGGATCCTGGGCGCCCGCC | | |
| | | | CGGCCGTTGCCACCGCGGTGTCCCGATTCTCGGGCGCC | | |
| | | | GCGATGATCCTCGTCGGTGCCCTGTTGCTGGTGGAGCG | | |
| | | | GCTGCTCGCCCAGGGGCCGACACATTAG | | |
| threonine | Corynebacterium | NC_003450 | GTGGACGCAGCATCATGGGTCGCATTCGCACTCGCATT | 275 | |
| efflux protein | glutamicum | | ATTGGTGGCATTAGCGGTGCCCGGACCTGACCTTGTTC | | |
| | | | TTGTTCTACATTCTGCAACCCGCGGGATCCGCACGGGG | | |
| | | | GTCATGACTGCGGCAGGAATCATGACGGGACTGATGTT | | |
| | | | ACATGCGAGTCTTGCGATAGCCGGAGCAACTGCATTAT | | |
| | | | TGCTATCAGCTCCGGGAGTATTGAGCGCTATTCAACTT | | |
| | | | CTTGGTGCGGGAGTGCTTTTGTGGATGGGCACGAACAT | | |
| | | | GTTTCGTGCTTCCCAAAATACCGGGGAATCTGAAACTG | | |
| | | | CTGCTAGTCAATCGAGTGCAGGTTATTTTCGAGGATTT | | |
| | | | ATCACCAATGCCACGAACCCGAAAGCGCTGTTGTTCTT | | |
| | | | TGCAGCGATTCTTCCTCAGTTCATTGGGAATGGGGAAG | | |
| | | | ATATGAAAATGAGGACCTTGGCATTGTGTGCCACCATC | | |
| | | | GTGCTTGGCTCAGGAGCGTGGTGGTTGGGAACAATCGC | | |
| | | | ATTGGTCAGGGGTATTGGTCTGCAAAAGTTACCGTCTG | | |
| | | | CGGATCGCATTATCACCCTGGTTGGTGGCATCGCACTG | | |
| | | | TTTCTCATTGGTGCCGGATTACTGGTTAATACTGCTTA | | |
| | | | TGGGCTTATCACT | | |
| hypo-thetical | Streptomyces | AL939116 | GTGTCGGTACCAGGGAGCGTTGCGCAGGTGACGGAGGC | 174 | |
| protein | coelicolor | | GGAGGAGCCCAAACCACAGTCGGACGAGGCCCGCAGTG | | |
| NCgl2533 | | | CCTTCCGGCAGCCCAGCGGGATCGCGGCGTCGATCGAC | | |
| related | | | GGCGAGTCGTCGACGACGTCCGAGTTCGAGATCCCGCA | | |
| | | | GGGGTTCGCCGTCCCGCGGCACGCCGGCACCGAGTCCG | | |
| | | | AGACGACCTCGGAGTTCTCGCTCCCCGACGGCCTGGAG | | |
| | | | GTGCCGCAGGCCCCGCCCGCGGACACCGAGGGCTCGGC | | |
| | | | ATTCACCATGCCGAGCACGCACAGCGCGTGGACCGCCC | | |
| | | | CGACCGCCTTCACCCCGGCGAGCGGCTTCCCGGCGGTG | | |
| | | | AGCCTGACGGACGTGCCCTGGCAGGACCGGATGCGCGC | | |
| | | | CATGCTGCGCATGCCGGTGGCCGAGCGGCCCGCGCCGG | | |
| | | | AGCCCTCGCAGAAGCACGACGACGAGACCGGCCCCGCC | | |
| | | | GTGCCGCGCGTGTTGGACCTGACGCTGCGTATCGGGGA | | |
| | | | GCTGCTGCTGGCGGGCGGTGAGGGCGCCGAGGACGTGG | | |
| | | | AGGCGGCCATGTTCGCCGTCTGCCGGTCCTACGGCCTG | | |
| | | | GACCGCTGCGAGCCGAACGTCACCTTCACCCTGCTGTC | | |
| | | | GATCTCCTACCAGCCGTCCCTGGTCGAGGACCCGGTGA | | |
| | | | CGGCGTCGCGGACGGTGCGCCGCCGCGGCACCGACTAC | | |
| | | | ACGCGGCTCGCGGCCGTCTTCCACCTGGTGGACGACCT | | |
| | | | CAGCGACCCCGACACGAACATCTCCCTGGAGGAGGCCT | | |
| | | | ACCGGCGTCTCGCGGAGATCCGCCGOAACCGCCACCCG | | |
| | | | TACCCCACCTGGGTGCTGACGGTGGCCAGCGGTCTGCT | | |
| | | | CGCGGGCGGGGCCTCGCTGCTCGTCGGTGGCGGGCTGA | | |
| | | | CCGTGTTCTTCGCGGCGATGTTCGGCTCGATGCTCGGC | | |
| | | | GACCGGCTGGCGTGGCTGTGCGCCGGGCGCGGGCTGCC | | |
| | | | GGAGTTCTACCAGTTCGCGGTGGCCGCGATGCCGCCCG | | |
| | | | CCGCGATGGGTGTCGTGCTGACGGTGACGCACGTCGAC | | |
| | | | GTGAAGGCGTCCGCGGTCATCACCGGTGGGCTGTTCGC | | |
| | | | GCTGCTGCCCGGGCGGGCGCTGGTCGCGGGGGTGCAGG | | |
| | | | ACGGTCTGACCGGCTTCTACATCACCGCCGCGGCCCGT | | |
| | | | CTGCTGGAGGTCATGTACTTCTTCGTCAGCATCGTCGC | | |
| | | | CGGGGTGCTGGTGGTGCTGTACTTCGGGGTCCAGCTGG | | |
| | | | GCGCCGAGCTCAACCCGGACGCCAAGCTCGGCACCGGT | | |
| | | | GACGAACCGTTCGTGCAGATCTTCGCCTCGATGCTGCT | | |
| | | | GTCGCTGGCCTTCGCGATCCTGCTCCAGCAGGAACGGG | | |
| | | | CCACCGTCCTCGCGGTGACCCTGAACGGCGGCATCGCC | | |
| | | | TGGTGCGTGTACGGCGCCATGAACTACGCCGGCGACAT | | |
| | | | CTCTCCGGTGGCCTCCACGGCCGCCGCGGCGGGGCTCG | | |
| | | | TGGGCCTGTTCGGGCAGCTGATGTCCAGGTACCGGTTC | | |
| | | | GCGTCGGCCCTGCCGTACACGACGGCGGCGATCGGGCC | | |
| | | | GCTGCTGCCCGGTTCGGCGACGTACTTCGGTCTGCTGG | | |
| | | | GGATCGCGCAGGGCGAGGTCGACTCGGGGCTGCTGTCG | | |
| | | | CTGTCCAACGCGGTGGCGCTGGCGATGGCCATCGCGAT | | |
| | | | CGGGGTGAACCTGGGCGGGGAGATCTCCCGGCTGTTCC | | |
| | | | TGAAGGTGCCCGGCGCCGCGAGTGCGGCGGGACGCCGG | | |
| | | | GCGGCCAAGCGGACGCGAGGGTTCTAG | | |
| hypo-thetical | Mycobacterium | AE007180 | ATGGATCAAGATCGATCGGACAACACGGCATTGCGCCG | 175 | |
| protein | tuberculosis | | TGGTCTGCGAATTGCCCTGCGCGGGCGCCGCGATCCGC | | |
| NCgl2533 | (use this to | | TGCCCGTGGCGGGCCGGCGGAGCCGGACCTCCGGCGGA | | |
| related | clone M. | | ATCGGTGACCTGCACACCCGGAAGGTGCTTGACCTGAC | | |
| | smegmatis | | CATCCGGCTCGCCGAGGTGATGTTGTCGTCCGGCTCTG | | |
| | gene) | | GCACCGCGGATGTCGTCGCCACAGCCCAGGACGTGGCT | | |
| | | | CAGGCCTACCAGCTCACCGATTGCGTTGTCGACATCAC | | |
| | | | CGTTACCACCATCATCGTGTCCGCGCTAGCGACCACAG | | |
| | | | ACACTCCGCCGGTCACCATCATGCGGTCGGTCCGGACC | | |
| | | | CGGTCCACTGACTACAGCCGGCTGGCCGAACTCGATCG | | |
| | | | ACTCGTTCAGCGGATAACCTCCGGTGGCGTCGCAGTCG | | |
| | | | ACCAGGCTCACGAGGCTATGGACGAGTTGACCGAACGG | | |
| | | | CCCCACCCCTACCCGCGCTGGCTCGCGACCGCGGGGGC | | |
| | | | GGCGGGCTTCGCACTCGGCGTCGCCATGTTGCTCGGCG | | |
| | | | GAACCTGGCTGACCTGCGTCTTGGCTGCCGTGACGTCT | | |
| | | | GGCGTGATCGACCGACTGGGCCGGCTGCTGAACCGGAT | | |
| | | | CGGGACCCCGTTGTTCTTCCAGCGCGTGTTCGGCGCGG | | |
| | | | GGATCGCGACCCTGGTCGCGGTGGCGGCTTACCTGATC | | |
| | | | GCCGGCCAGGATCCGACCGCGCTGGTGGCCACCGGAAT | | |
| | | | CGTTGTGCTGCTGTCTGGGATGACCTTGGTGGGTTCGA | | |
| | | | TGCAGGACGCGGTCACCGGGTACATGCTCACCGCACTC | | |
| | | | GCCCGGCTTGGCGACGCCCTGTTCCTGACCGCAGGGAT | | |
| | | | CGTCGTCGGCATCCTCATCTCGTTGCGGGGCGTCACCA | | |
| | | | ATGCCGGCATCCAGATCGAACTGCATGTCGACGCAACC | | |
| | | | ACGACGCTCGCCACCCCGGGCATGCCGCTACCGATTCT | | |
| | | | CGTCGCGGTAAGCGGTGCGGCGCTGTCCGGCGTGTGCC | | |
| | | | TGACGATCGCGAGCTATGCGCCGCTACGTTCTGTGGCC | | |
| | | | ACCGCCGGACTCTCGGCCGGACTCGCCGAACTGGTGCT | | |
| | | | CATCGGACTCGGCGCGGCCGGGTTCGGCCGAGTGGTCG | | |
| | | | CCACCTGGACCGCCGCGATCGGCGTCGGCTTCTTGGCC | | |
| | | | ACCCTGATCTCAATCCGTCGGCAGGCTCCCGCCTTGGT | | |
| | | | GACGGCCACCGCCGGCATCATGCCGATGCTGCCGGGCC | | |
| | | | TTGCGGTCTTCCGTGCCGTGTTCGCGTTCGCCGTCAAT | | |
| | | | GACACACCCGACGGCGGTCTGACCCAGCTGCTGGAAGC | | |
| | | | GGCCGCGACTGCACTCGCGCTTGGCAGCGGGGTGGTGT | | |
| | | | CGGGCGAGTTCCTCGCCTCACCATTGCGGTACGGCGCC | | |
| | | | AGCCGGATCGGCGACCTCTTTCGGATCGAGGGTCCACC | | |
| | | | CGGGCTCCGGCGGGCGGTCGGCCGTGTGGTGCGCCTAC | | |
| | | | AGCCGGCCAAGAGCCAGCAGCCGACCGGCACCGGTGGC | | |
| | | | CAACGGTGGCGAAGCGTCGCGCTGGAGCCGACGACGGC | | |
| | | | CGACGACGTGGACGCCGGCTATCGCGGCGATTGGCCCG | | |
| | | | CTACCTGCACCAGCGCGACCGAGGTGCGCTAG | | |
| hypo-thetical | Mycobacterium | AL022121 | ATGGATCAAGATCGATCGGACAACACGGCATTGCGCCG | 176 | |
| protein | tuberculosis | | TGGTCTGCGAATTGCCCTGCGCGGGCGCCGCGATCCGC | | |
| NCgl2533 | (use this to | | TGCCCGTGGCGGGCCGGCGGAGCCGGACCTCCGGCGGA | | |
| related | clone M. | | ATCGATGACCTGCACACCCGGAAGGTGCTTGACCTGAC | | |
| | smegmatis | | CATCCGGCTCGCCGAGGTGATGTTGTCGTCCGGCTCTG | | |
| | gene) | | GCACCGCGGATGTCGTCGCCACAGCCCAGGACGTGGCT | | |
| | | | CAGGCCTACCAGCTCACCGATTGCGTTGTCGACATCAC | | |
| | | | CGTTACCACCATCATCGTGTCCGCGCTAGCGACCACAG | | |
| | | | ACACTCCGCCGGTCACCATCATGCGGTCGGTCCGGACC | | |
| | | | CGGTCCACTGACTACAGCCGGCTGGCCGAACTCGATCG | | |
| | | | ACTCGTTCAGCGGATAACCTCCGGTGGCGTCGCAGTCG | | |
| | | | ACCAGGCTCACGAGGCTATGGACGAGTTGACCGAACGG | | |
| | | | CCCCACCCCTACCCGCGCTGGCTCGCGACCGCGGGGGC | | |
| | | | GGCGGGCTTCGCACTCGGCGTCGCCATGTTGCTCGGCG | | |
| | | | GAACCTGGCTGACCTGCGTCTTGGCTGCCGTGACGTCT | | |
| | | | GGCGTGATCGACCGACTGGGCCGGCTGCTGAACCGGAT | | |
| | | | CGGGACCCCGTTGTTCTTCCAGCGCGTGTTCGGCGCGG | | |
| | | | GGATCGCGACCCTGGTCGCGGTGGCGGCTTACCTGATC | | |
| | | | GCCGGCCAGGATCCGACCGCGCTGGTGGCCACCGGAAT | | |
| | | | CGTTGTGCTGCTGTCTGGGATGACCTTGGTGGGTTCGA | | |
| | | | TGCAGGACGCGGTCACCGGGTACATGCTCACCGCACTC | | |
| | | | GCCCGGCTTGGCGACGCCCTGTTCCTGACCGCAGGGAT | | |
| | | | CGTCGTCGGCATCCTCATCTCGTTGCGGGGCGTCACCA | | |
| | | | ATGCCGGCATCCAGATCGAACTGCATGTCGACGCAACC | | |
| | | | ACGACGCTCGCCACCCCGGGCATGCCGCTACCGATTCT | | |
| | | | CGTCGCGGTAAGCGGTGCGGCGCTGTCCGGCGTGTGCC | | |
| | | | TGACGATCGCGAGCTATGCGCCGCTACGTTCTGTGGCC | | |
| | | | ACCGCCGGACTCTCGGCCGGACTCGCCGAACTGGTGCT | | |
| | | | CATCGGACTCGGCGCGGCCGGGTTCGGCCGAGTGGTCG | | |
| | | | CCACCTGGACCGCCGCGATCGGCGTCGGCTTCTTGGCC | | |
| | | | ACCCTGATCTCAATCCGTCGGCAGGCTCCCGCCTTGGT | | |
| | | | GACGGCCACCGCCGGCATCATGCCGATGCTGCCGGGCC | | |
| | | | TTGCGGTCTTCCGTGCCGTGTTCGCGTTCGCCGTCAAT | | |
| | | | GACACACCCGACGGCGGTCTGACCCAGCTGCTGGAAGC | | |
| | | | GGCCGCGACTGCACTCGCGCTTGGCAGCGGGGTGGTGT | | |
| | | | TGGGCGAGTTCCTCGCCTCACCATTGCGGTACGGCGCC | | |
| | | | GGCCGGATCGGCGACCTCTTTCGGATCGAGGGTCCACC | | |
| | | | CGGGCTCCGGCGGGCGGTCGGCCGTGTGGTGCGCCTAC | | |
| | | | AGCCGGCCAAGAGCCAGCAGCCGACCGGCACCGGTGGC | | |
| | | | CAACGGTGGCGAAGCGTCGCGCTGGAGCCGACGACGGC | | |
| | | | CGACGACGTGGACGCCGGCTATCGCGGCGATTGGCCCG | | |
| | | | CTACCTGCACCAGCGCGACCGAGGTGCGCTAG | | |
| hypo-thetical | Thermobifida | NZ_AAAQ010 | GTGATCTCATACGGTCCGGTGGCGGATCGGTGCAGGGT | 177 | |
| protein | fusca | 00042 | GGGGGCAACTTCGGCGGCGTGGGGAACGTCTCCCCCAA | | |
| NCgl2533 | | | TGAGCTTTCCGTTTCTTCCCCTTGTATCCCACCCACTC | | |
| related | | | CCTTATGTCCCAGGTTTGGATGCGTCATTCCCGGATGG | | |
| | | | AGCATGCGTCCCGTTGGGCAGGGGTCCCTCCCGAGGAG | | |
| | | | GTGAGCGCCGGATGAACCAGGCACCGCGGCGTTCCGAC | | |
| | | | ACATCGCACTCCCCCACCCTGCTGACCCGGTTGCGGGA | | |
| | | | CTGGCGTGCCAGCCGCGGCGTGCTCGACCTGGAAGCAG | | |
| | | | AAGAGTTCGAAGACGAAGCGCCGCGTCCCGATCCGCGG | | |
| | | | GCCATGGACCTCGTCCTGCGGGTAGGGGAACTGCTGCT | | |
| | | | GGCCAGCGGGGAAGCCACCGAGACGGTCAGCGACGCGA | | |
| | | | TGCTGAGTCTGGCGGTGGCGTTCGAATTGCCCCGCAGC | | |
| | | | GAAGTGTCGGTGACGTTCACCGGCATCACCCTGTCGTG | | |
| | | | CCACCCCGGCGGGGATGAGCCCCCGGTGACCGGGGAGC | | |
| | | | GCGTGGTGCGCCGCCGCTCCCTCGACTACCACAAGGTC | | |
| | | | AACGAGCTGCACGCGCTGGTGGAAGACGCTGCGTTGGG | | |
| | | | CCTGCTCGACGTGGAGCGCGCAACCGCGCGGCTCCACG | | |
| | | | CCATCAAACGCTCCCGGCCGCACTATCCCCGCTGGGTG | | |
| | | | ATCGTGGCCGGGCTGGGGCTGATCGCCAGCAGCGCCAG | | |
| | | | TGTCATGGTGGGCGGTGGGATCATCGTGGCGGCCACGG | | |
| | | | CGTTCGCCGCCACCGTGCTCGGGGACCGGGCCGCGGGC | | |
| | | | TGGCTGGCTCGACGCGGGGTGGCCGAGTTCTACCAGAT | | |
| | | | GGCGGTGGCCGCGCTGTTGGCGGCGAGCACCGGCATGG | | |
| | | | CGCTGCTGTGGGTGAGCGAGGAGCTGGAGTTGGGGCTT | | |
| | | | CGCGCGAACGCGGTGATCACCGGGAGCATTGTGGCGCT | | |
| | | | GCTACCGGGGCGTCCCCTGGTCTCCAGCCTGCAAGACG | | |
| | | | GGATCAGCGGCGCGTACGTGTCGGCGGCGGCCCGCCTC | | |
| | | | TTGGAGGTCTTCTTCATGTTGGGGGCGATCGTCGCGGG | | |
| | | | GGTTGGCGCGGTCGCCTATACCGCGGTGCGGCTAGGGC | | |
| | | | TTTATGTGGACCTCGACAATCTGCCGTCGGCGGGGACG | | |
| | | | TCACTGGAGCCGGTCGTGCTGGCAGCTGCGGCAGGTTT | | |
| | | | GGCGCTCGCGTTCGCGGTGTCCCTGGTCGCGCCGGTGC | | |
| | | | GGGCCCTGCTGCCGATCGGCGCGATGGGGGTGCTGATC | | |
| | | | TGGGTGTGCTATGCGGGGCTGCGGGAACTGCTCGCCGT | | |
| | | | GCCGCCTGTGGTGGGGACCGGGGCGGGCGCGGTCGTGG | | |
| | | | TCGGGGTGATCGGCCACTGGCTGGCCCGGCGGACCCGG | | |
| | | | CGTCCTCCGCTCACCTTCATCATTCCGTCGATCGCTCC | | |
| | | | GCTGCTGCCGGGAAGCATCCTGTACCGGGGACTGATCG | | |
| | | | AGATGAGCACGGGGGAGCCGCTGGCCGGGGTGGCGAGC | | |
| | | | CTCGGTGAGGCGGTCGCGGTCGGCCTGGCTCTGGGTGC | | |
| | | | GGGGGTGAACCTCGGTGGTGAGCTGGTGCGGGCCTTCT | | |
| | | | CGTGGGGCGGTCTCGTGGGTGCGGGGCGCCGGGGTCGG | | |
| | | | CAGGCGGCCCGCCGGACCCGGGGAGGCTACTAG | | |
| hypo-thetical | Lactobacillus | AL935252 | ATGAATAAAGAGCGTAAGTCGGTGATGCCGCTATCACA | 178 | |
| protein | plantarum | | ACGACATCATATGACAATTCCATGGAAGGACTTTATCC | | |
| NC9l2533 | | | GTAATGAAGATGTTCCCGCTAAGCATGCTAGCTTACAA | | |
| related | | | GAGCGAACATCAATTGTTGGTCGAGTTGGTATTTTAAT | | |
| | | | GTTGTCGTGTGGGACGGGAGCGTGGCGGGTTCGTGATG | | |
| | | | CGATGAATAAGATTGCTCGCAGCCTGAATTTAACGTGC | | |
| | | | TCGGCAGATATCGGGTTGATTTCGATTCAGTACACGTG | | |
| | | | TTTTCATCATGAACGTAGTTATACGCAAGTATTATCGA | | |
| | | | TACCAAATACTGGTGTAAATACGGATAAACTAAATATT | | |
| | | | CTTGAACAGTTTGTCAAAGACTTTGATGCGAAATATGC | | |
| | | | ACGGTTAACGGTGGCACAAGTGCATGCAGCAATTGATG | | |
| | | | AAGTTCAGACGCGTCCTAAACAGTATTCGCCACTGGTT | | |
| | | | CTTGGGTTGGCAGCTGGCTTAGCCTGTAGTGGATTTAT | | |
| | | | CTTCTTACTTGGTGGAGGTATTCCCGAGATGATTTGTT | | |
| | | | CCTTTTTGGGCGCGGGCCTTGGTAACTATGTTCGGGCG | | |
| | | | CTGATGGGTAAACGGTCGATGACGACGGTTGCCGGGAT | | |
| | | | TGCGGTCAGCGTTGCGGTAGCGTGTTTGGCTTATATGG | | |
| | | | TTAGTTTTAAGATTTTTGAATATAATTTCCAAATTCTT | | |
| | | | GCCCAGCATGAGGCGGGGTATATTGGTGCCATGTTATT | | |
| | | | CGTGATTCCGGGTTTTCCGTTCATTACGAGTATGTTGG | | |
| | | | ATATCTCTAAGTTGGATATGCGCTCAGGACTGGAGCGC | | |
| | | | TTAGCTTACGCGATTATGGTTACCCTGATTGCAACTCT | | |
| | | | CGTCGGCTGGCTAGTCGCGACACTGGTGAGCTTCAAGC | | |
| | | | CAGCTGATTTCTTACCGCTAGGACTTTCACCGTTAGCG | | |
| | | | GTACTTTTATTACGATTACCAGCTAGTTTTTGCGGTGT | | |
| | | | TTACGGGTTCTCAATAATGTTTAATAGCTCGCAAAAAA | | |
| | | | TGGCCATTACCGCGGGATTTATTGGGGCCATTGCGAAT | | |
| | | | ACATTGCGCCTTGAACTAGTTGACTTGACAGCAATGCC | | |
| | | | ACCGGCCGCGGCCGCCTTTTGTGGGGCGCTCGTTGCCG | | |
| | | | GCTTGATCGCATCGGTGGTTAATCGTTATAACGGCTAT | | |
| | | | CCCCGGATTTCATTGACGGTACCTTCAATCGTAATTAT | | |
| | | | GGTTCCGGGATTATATATTTATCGTGCAATTTATAGTA | | |
| | | | TTGGCAATAATCAAATTGGTGTCGGTTCACTATGGCTG | | |
| | | | ACGAAGGCCGTGTTAATCATCATGTTTTTACCGCTCGG | | |
| | | | GCTATTTGTAGCGCGTGCGTTGTTGGATCACGAATGGC | | |
| | | | GACACTTTGATTAA | | |
| NCgl2533 | Coryne- | NC_003450 | ATGTTGAGTTTTGCGACCCTTCGTGGCCGCATTTCAAC | 276 | |
| | | bacterium | | AGTTGACGCTGCAAAAGCCGCACCTCCGCCATCGCCAC | |
| | glutamicum | | TAGCCCCGATTGATCTCACTGACCATAGTCAAGTGGCC | | |
| | | | GGTGTGATGAATTTGGCTGCGAGAATTGGCGATATTTT | | |
| | | | GCTTTCTTCAGGTACGTCAAATAGTGACACCAAGGTAC | | |
| | | | AAGTTCGAGCAGTGACCTCTGCGTACGGTTTGTACTAC | | |
| | | | ACGCACGTGGATATCACGTTGAATACGATCACCATCTT | | |
| | | | CACCAACATCGGTGTGGAGAGGAAGATGCCGGTCAACG | | |
| | | | TGTTTCATGTTGTAGGCAAGTTGGACACCAACTTCTCC | | |
| | | | AAACTGTCTGAGGTTGACCGTTTGATCCGTTCCATTCA | | |
| | | | GGCTGGTGCGACCCCGCCTGAGGTTGCCGAGAAAATCC | | |
| | | | TGGACGAGTTGGAGCAATCCCCTGCGTCTTATGGTTTC | | |
| | | | CCTGTTGCGTTGCTTGGCTGGGCAATGATGGGTGGTGC | | |
| | | | TGTTGCTGTGCTGTTGGGTGGTGGATGGCAGGTTTCCC | | |
| | | | TAATTGCTTTTATTACCGCGTTCACGATCATTGCCACG | | |
| | | | ACGTCATTTTTGGGAAAGAAGGGTTTGCCTACTTTCTT | | |
| | | | CCAAAATGTTGTTGGTGGTTTTATTGCCACGCTGCCTG | | |
| | | | CATCGATTGCTTATTCTTTGGCGTTGCAATTTGGTCTT | | |
| | | | GAGATCAAACCGAGCCAGATCATCGCATCTGGAATTGT | | |
| | | | TGTGCTGTTGGCAGGTTTGACACTCGTGCAATCTCTGC | | |
| | | | AGGACGGCATCACGGGCGCTCCGGTGACAGCAAGTGCA | | |
| | | | CGATTTTTCGAAACACTCCTGTTTACCGGCGGCATTGT | | |
| | | | TGCTGGCGTGGGTTTGGGCATTCAGCTTTCTGAAATCT | | |
| | | | TGCATGTCATGTTGCCTGCCATGGAGTCCGCTGCAGCA | | |
| | | | CCTAATTATTCGTCTACATTCGCCCGCATTATCGCTGG | | |
| | | | TGGCGTCACCGCAGCGGCCTTCGCAGTGGGTTGTTACG | | |
| | | | CGGAGTGGTCCTCGGTGATTATTGCGGGGCTTACTGCG | | |
| | | | CTGATGGGTTCTGCGTTTTATTACCTCTTCGTTGTTTA | | |
| | | | TTTAGGCCCCGTCTCTGCCGCTGCGATTGCTGCAACAG | | |
| | | | CAGTTGGTTTCACTGGTGGTTTGCTTGCCCGTCGATTC | | |
| | | | TTGATTCCACCGTTGATTGTGGCGATTGCCGGCATCAC | | |
| | | | ACCAATGCTTCCAGGTCTAGCAATTTACCGCGGAATGT | | |
| | | | ACGCCACCCTGAATGATCAAACACTCATGGGTTTCACC | | |
| | | | AACATTGCGGTTGCTTTAGCCACTGCTTCATCACTTGC | | |
| | | | CGCTGGCGTGGTTTTGGGTGAGTGGATTGCCCGCAGGC | | |
| | | | TACGTCGTCCACCACGCTTCAACCCATACCGTGCATTT | | |
| | | | ACCAAGGCGAATGAGTTCTCCTTCCAGGAGGAAGCTGA | | |
| | | | GCAGAATCAGCGCCGGCAGAGAAAACGTCCAAAGACTA | | |
| | | | ATCAGAGATTCGGTAATAAAAGG | | |
| putative | Thermobifida | NZ_AAAQ010 | ATGTCAGGGGGAGTCATGGCCGACATCACCAGAAACCG | 179 | |
| mem-brane | fusca | 00018 | GTCCTCCGGGTTGGCATTCGCGATCGCCTCTGCACTTG | | |
| protein | | | CCTTCGGCGGCTCCGGCCCCGTGGCCCGGCCGCTCATC | | |
| NCgL0580 | | | GACGCCGGACTCGACCCCCTGCACGTCACGTGGCTCCG | | |
| related | | | GGTAGCCGGAGCAGCTCTACTCCTGCTTCCCGTCGCTT | | |
| | | | TCCGCCACCACCGCACCCTGCGTACCCGCCCCGCCCTT | | |
| | | | CTCCTCGCCTACGGCGTCTTCCCGATGGCGGGAGTCCA | | |
| | | | AGCCTTCTACTTCGCAGCCATTTCCCGGATCCCCGTGG | | |
| | | | GGGTGGCGCTCCTCATCGAATTCCTCGGCCCCGTCCTC | | |
| | | | GTCCTGCTGTGGACCCGCCTCGTGCGGCGCATCCCCGT | | |
| | | | GTCCCGCGCCGCATCCCTCGGCGTGGCCCTGGCAGTCA | | |
| | | | TCGGCCTGGGCTGCCTCGTCGAAGTCTGGGCAGGCATC | | |
| | | | CGCCTGGACGCGGTCGGCCTGATCCTCGCGCTGGCTGC | | |
| | | | AGCGGTCTGCCAGGCCACCTACTTCCTGCTGTCGGACA | | |
| | | | CGGCCCGCGACGACGTCGACCCTCTCGCTGTCATCTCC | | |
| | | | TACGGCGCGCTCATCGCCACCGCACTCCTGAGCCTCCT | | |
| | | | CGCCCGCCCGTGGACCCTGCCGTGGGGCATCCTGGCCC | | |
| | | | AGAATGTCGGGTTCGGCGGGCTGGACATCCCCGCCCTC | | |
| | | | ATCCTCCTGGTGTGGCTTGCCCTGGTCGCCACCACCAT | | |
| | | | CGCCTACCTCACCGGGGTGGCCGCGGTACGGCGGCTGT | | |
| | | | CCCCTGTCGTCGCCGGGGGAGTGGCCTACCTGGAGGTC | | |
| | | | GTAACCTCTATCGTCCTGGCCTGGCTGCTGCTCGGGGA | | |
| | | | AGCGTTGAGCGTCGCCCAGCTTGTCGGGGCGGCCGCCG | | |
| | | | TGGTGACCGGTGCGTTCCTCGCCCAGACCGCGGTCCCC | | |
| | | | GACACCAGTGCCGCGCAAGGCCCGGAGACGCTGCCCAC | | |
| | | | CGCCCAGGACCCGGCCCCGCAGACCGGTTCCGCCCGCT | | |
| | | | GA | | |
| putative | Thermobifida | NZ_AAAQ010 | GTGAATAGCGACTCTCCTGGGCAGTCTGCACCGGGTCC | 180 | |
| mem-brane | fusca | 00042 | GTTCTCCCGGGCTGCGGCGCTCGTCCGCGCCGCGGGCA | | |
| protein | | | CTGCCATCCCGGCGACCTGGCTGGTCGGGGTGAGCATC | | |
| NCgl0580 | | | CTGTCGGTCCAGTTCGGCGCAGGGGTGGCGAAGAACCT | | |
| related | | | GTTCGCGGTCCTCCCCCCAAGCACCGTGGTGTGGCTGC | | |
| | | | GCCTGCTGGCTTCGGCCCTGGTGCTGCTGTGCTTCGCC | | |
| | | | CCTCCCCCACTGCGCGGGCACTCTCGCACGGACTGGCT | | |
| | | | GGTCGCGGTCGGTTTCGGCACGTCGCTGGCGGTCATGA | | |
| | | | ACTACGCCATCTACGAATCGTTTGCGCGCATCCCGCTG | | |
| | | | GGCGTGGCCGTGACCATCGAATTCCTGGGCCCGCTGGC | | |
| | | | CGTGGCCGTGGCGGGATCGCGCCGCTGGCGGGACCTGG | | |
| | | | TGTGGGTGGTGCTCGCCGGCACGGGGGTTGCGCTGCTG | | |
| | | | GGATGGGACGACGGCGGGGTCACCCTGGCAGGGGTGGC | | |
| | | | GTTCGCCGCCCTCGCGGGCGCTGCGTGGGCGTGCTAcA | | |
| | | | TCCTGCTCAGCGCAGCCACCGGCCGACGCTTCCCCGGG | | |
| | | | ACTTCCGGACTGACGGTGGCCAGTGTGATCGGCGCAGT | | |
| | | | GCTCGTCGCGCCGATGGGCCTCGCCCACAGCAGCCCGG | | |
| | | | CCCTGCTCGACCCGAGCGTGCTGCTGACCGGTCTTGCC | | |
| | | | GTGGGGCTGCTCTCCTCGGTCATCCCCTACTCCCTGGA | | |
| | | | AATGCAGGCGTTGCGCCGCATTCCGCCCGGGGTGTTCG | | |
| | | | GCATCCTGATGAGCCTAGAACCGGCGGCGGCCGCACTC | | |
| | | | GTGGGCCTGGTCCTGCTCGGGGAATTCCTCACCGTCGC | | |
| | | | CCAGTGGGCCGCGGTGGCCTGCGTGGTGGTCGCCAGTG | | |
| | | | TGGGTGCGACCCGCTCCGCCCGGCTGTGA | | |
| putative | Thermobifida | NZ_AAAQ010 | GTGTGGACGCTAGATCTTCCGCTAAAGAGAAACGATTC | 181 | |
| mem-brane | fusca | 00033 | ATCAACTAACGGTGCCTGGACGGAAACAGAGAATAGGA | | |
| protein | | | GACACAGTGGTGGGATGATCCTCTCTTTTGTCTCGTTG | | |
| NCgl0580 | | | GTTCGGCATGCCCACCTGAGGGTCCCAGCCCCGCTGCT | | |
| related | | | CACCGTCCTCAGCCTGGTCCTGCTGCACATGGGCAGCG | | |
| | | | CGGGAGCCGTGCACCTGTTCGCCATCGCGGGACCGCTC | | |
| | | | GAAGTCACCTGGCTGCGGCTGAGCTGGGCTGCGCTCCT | | |
| | | | CCTCTTCGCCGTCGGCGGGCGCCCCCTGCTCCGCGCGG | | |
| | | | CACGGGCCGCAACCTGGTCGGATCTCGCCGCTACCGCC | | |
| | | | GCCCTCGGCGTAGTCAGCGCGGGGATGACCCTCCTGTT | | |
| | | | CTCCCTCGCCCTCGACCGCATCCCGCTCGGCACCGCAG | | |
| | | | CCGCGATCGAGTTCCTCGGCCCCCTCACCGTCTCCGTG | | |
| | | | CTCGCCCTGCGCCGCCGCCGCGACCTGCTGTGGATCGT | | |
| | | | CCTCGCCGTAGCCGGAGTGCTCCTGCTCACCCGCCCGT | | |
| | | | GGCACGGGGAAGCCGACCTGCTCGGCATCGCCTTCGGC | | |
| | | | CTAGGCGGGGCCGTCTGCGTGGCGCTCTACATCGTCTT | | |
| | | | CTCCCAGACCGTCGGCTCCCGGCTGGGCGTCCTCCCCG | | |
| | | | GCCTCACCCTCGCAATGACCGTGTCCGCCCTGGTCACC | | |
| | | | GCCCCGCTGGGTCTGCCGGGGGCGATGGCGGCCGCCGA | | |
| | | | CCGGCACCTGGTGGCAGCCACCCTAGGGCTCGCACTGA | | |
| | | | TCTACCCCCTGCTGCCCCTCCTGCTGGAGATGGTGAGC | | |
| | | | CTGCAACGGATGAACCGCGGCACCTTCGGCATTCTCGT | | |
| | | | CTCCGTCGACCCCGCCATCGGGCTGCTCATCGGCCTGC | | |
| | | | TCCTGATCGGCCAGGTCCCCGTCCCCCTCCAAGTGGCG | | |
| | | | GGCATGGCCCTGGTGGTCGCCGCCGGGCTGGGCGCCAC | | |
| | | | CAGAGGCACCAGCGGACGCACACGCGGAGGCGCAGACC | | |
| | | | CGCACGCCACCGACGGGGAGCCGGAAGACCGCACCCCG | | |
| | | | GACCGCCCTGCTCCCGACGACGCCGGGCACCACACCAC | | |
| | | | CGACCCCGTCACAGTGTGA | | |
| putative | Streptomyces | SC0939113 | ATGGCCGCCACCCGCCCCGCCGTCATCGCGCTCACCGC | 182 | |
| mem-brane | coelicolor | | CCTCGCCCCCGTCTCCTGGGGCAGCACCTACGCCGTGA | | |
| protein | | | CCACCGAGTTCCTGCCGCCCGACCGGCCCCTGTTCACC | | |
| NCgl0580 | | | GGGCTGATGCGGGCTCTGCCCGCCGGCCTGCTGCTGCT | | |
| related | | | CGCCCTCGCCCGGGTGCTGCCGCGCGGCGCCTGGTGGG | | |
| | | | GGAAGGCGGCGGTGCTGGGGGTGCTGAACATCGGGGCC | | |
| | | | TTCTTCCCGCTGCTGTTCCTCGCCGCCTACCGGATGCC | | |
| | | | CGGCGGAATGGCCGCCGTCGTCGGCTCGGTCGGCCCGC | | |
| | | | TCCTCGTCGTCGGCCTCTCGGCCCTCCTGCTCGGGCAG | | |
| | | | CGGCCCACCACCCGGTCCGTTCTCACCGGTGTCGCCGC | | |
| | | | CGCGTCCGGCGTCAGCCTGGTGGTGCTGGAGGCGGCCG | | |
| | | | GGGCGCTGGACCCGCTCGGCGTGCTGGCGGCCCTCGCC | | |
| | | | GCCACCGCCTCCATGTCCACCGGCACCGTGCTCGCGGG | | |
| | | | GCGCTGGGGCCGCCCCGAAGGCGTCGGCCCGCTCGCCC | | |
| | | | TCACCGGCTGGCAACTGACCGCGGGCGGCCTGCTCCTG | | |
| | | | GCACCGCTCGCCCTGCTGGTCGAGGGTGCCCCGCCCGC | | |
| | | | CCTGGACGGCCCGGCCGTCGGCGGCTACCTCTACCTGG | | |
| | | | CGCTGGCCAACACGGCGCTGGCGTACTGGCTCTGGTTC | | |
| | | | CGCGGCATCGGCCGGCTCTCGGCCACTCAGGTCACCTT | | |
| | | | CCTCGGACCGCTCTCGCCGCTGACCGCCGCCGTGATCG | | |
| | | | GCTGGGCGGCACTCGGCGAGGCGCTCGGCCCGGTGCAA | | |
| | | | CTGGCGGGGACGGCGCTGGCCTTCGGAGCGACCCTCGT | | |
| | | | GGGCCAGACGGTACCGAGCGCGCCGCGCACGCCGCCGG | | |
| | | | TCGCCGCGGGCGCCGGTCCGTTCAGTTCTGCTTCACGA | | |
| | | | AACGGTCGAAAAGATTCGATGGACCTGACGGGTGCGGC | | |
| | | | CCTGCGACGGTAG | | |
| putative | Streptomyces | AL939119 | ATGCCGGACGGCGCGCCGGGCGGACGGTTCGGCGCCCT | 183 | |
| mem-brane | coelicolor | | CGGACCCGTCGGCCTGGTCCTCGCCGGTGGCATCTCCG | | |
| protein | | | TGCAGTTCGGCGCCGCGCTGGCGGTGAGTCTGATGCCG | | |
| NCgl0580 | | | CGGGCCGGGGCGCTCGGCGTGGTGACCCTGCGGCTCGC | | |
| related | | | CGTGGCCGCCGTCGTCATGCTCCTGGTCTGCCGGCCCC | | |
| | | | GGCTGCGCGGCCACTCCCGGGCCGACTGGGGCACGGTC | | |
| | | | GTCGTCTTCGGCATCGCCATGGCCGGCATGAACGGCCT | | |
| | | | CTTCTACCAGGCCGTCGACCGCATCCCGCTCGGCCCCG | | |
| | | | CGGTCACCCTGGAGGTGCTCGGCCCGCTCGCCCTGTCC | | |
| | | | GTCTTCGCCTCCCGCCGTGCGATGAACCTGGTCTGGGC | | |
| | | | CGCGCTCGCCCTGGCCGGTGTCTTCCTGCTGGGCGGCG | | |
| | | | GCGGCTTCGACGGCCTCGACCCGGCCGGTGCCGCCTTC | | |
| | | | GCCCTGGCGGCGGGCGCCATGTGGGCGGCGTACATCGT | | |
| | | | CTTCAGTGCCCGCACCGGACGCCGCTTCCCGCAGGCCG | | |
| | | | ACGGGCTGGCGCTGGCGATGGCGGTCGGCGCGCTGCTG | | |
| | | | TTCCTGCCGCTCGGCATCGTCGAGTCGGGGTCGAAGCT | | |
| | | | GATCGACCCGGTGACGCTCACGCTGGGCGCCGGCGTCG | | |
| | | | CCCTGCTCTCCTCCGTCCTGCCCTACACCCTCGAACTC | | |
| | | | CTCGCGCTGCGCCGTCTGCCAGCGCCGACCTTCGCCAT | | |
| | | | CCTCATGAGCCTGGAGCCCGCCATCGCCGCGGCGGCCG | | |
| | | | GTTTCCTCATCCTCGACCAGGCACTGACCGCCACCCAG | | |
| | | | TCCGCCGCCATCGCCCTGGTCATCGCGGCGAGCATGGG | | |
| | | | AGCGGTGCGGACCCAGGTGGGGCGGCGCCGGGCGAAGG | | |
| | | | CGCTTCCCGAGTAG | | |
| putative | Streptomyces | AL939110 | ATGATGACCACCGCCCGCACGTCCCCTCCCGCCCCCTG | 184 | |
| mem-brane | coelicolor | | GCACCGTCGTCCCGACCTGCTCGCGGCCGGCGCGGCCA | | |
| protein | | | CCGTCACCGTCGTGCTGTGGGCATCCGCGTTCGTCTCC | | |
| NCgl0580 | | | ATCCGCAGCGCGGGCGAGGCGTACTCGCCGGGCGCGCT | | |
| related | | | GGCGCTCGGCCGGCTGCTGTCGGGCGTCCTGACGCTCG | | |
| | | | GGGCGATCTGGCTGCTGCGCCGGGAGGGGCTGCCGCcG | | |
| | | | CGCGCGGCCTGGCGGGGGATCGCGATATCGGGGCTGCT | | |
| | | | GTGGTTCGGGTTCTACATGGTCGTCCTGAACTGGGGCG | | |
| | | | AGCAGCAGGTGGACGCCGGCACGGCCGCCCTCGTGGTC | | |
| | | | AACGTCGGCCCGATCCTCATCGCGCTGCTCGGCGCGCG | | |
| | | | GCTGCTGGGCGACGCGCTGCCGCCACGGCTGTTGACGG | | |
| | | | GGATGGCGGTGTCGTTCGCCGGTGCGGTGACCGTGGGC | | |
| | | | CTGTCCATGTCCGGCGAGGGCGGTTCCTCGCTGTTCGG | | |
| | | | GGTGGTGCTGTGCCTGCTGGCCGCGGTGGCGTACGCGG | | |
| | | | GCGGGGTGGTGGCCCAGAAGCCCGCGCTGGCGCACGCG | | |
| | | | AGCGCCCTTCAGGTGACGACGTTCGGGTGCCTGGTCGG | | |
| | | | GGCGGTGCTCTGCCTGCCGTTCGCCGGGCAGCTGGTGC | | |
| | | | ACGAGGCGGCCGGCGCGCCGGTCTCCGCCACGCTCAAC | | |
| | | | ATGGTCTACCTGGGCGTGTTCCCGACCGCCCTGGCGTT | | |
| | | | CACGACGTGGGCCTACGCCCTGGCCCGTACGACCGCCG | | |
| | | | GCCGCATGGGTGCGACCACGTACGCCGTGCCCGCGCTG | | |
| | | | GTCGTGCTGATGTCGTGGCTGGCACTGGGCGAGGTCCC | | |
| | | | GGGGCTGCTCACCCTGGCGGGCGGAGCGCTGTGCCTGG | | |
| | | | CGGGCGTGGCCGTGTCCCGCTCGCGCAGGCGCCCGGCC | | |
| | | | GCGGTCCCCGACCGGGCCGCGCCCACGGCGGAGCCACG | | |
| | | | GCGCGAGGACGCGGGGCGGGCCTAG | | |
| putative | Streptomyces | AL939108 | GTGCCGGTGCATACGTCTGACAGCGCCCGCGGCAGCCG | 185 | |
| mem-brane | coelicolor | | CGGCAAGGGCATCGGGCTCGGCCTGGCACTGGCCTCCG | | |
| protein | | | CGGTCGCCTTCGGAGGTTCCGGAGTCGCGGCCAAACCG | | |
| NCgl0580 | | | CTCATCGAGGCCGGGCTCGATCCGCTCCACGTGGTCTG | | |
| related | | | GCTGCGCGTCGCGGGCGCGGCCCTGGTGATGCTGCCGC | | |
| | | | TCGCCGTGCGCCACCGCGCCCTGCCGCGCCGCCGTCCC | | |
| | | | GCGCTGGTCGCCGGGTACGGACTGTTCGCCGTGGCCGG | | |
| | | | TGTCCAGGCGTGCTACTTCGCGGCCATCTCGCGCATCC | | |
| | | | CCGTCGGCGTCGCCCTGCTGGTCGAGTACCTGGCGCCC | | |
| | | | GCTCTGGTCCTCGGCTGGGTGCGGTTCGTGCAACGGCG | | |
| | | | GCCGGTCACACGCGCCGCCGCGCTCGGCGTGGTCCTGG | | |
| | | | CGGTCGGCGGCCTCGCCTGCGTGGTCGAGGTCTGGTCG | | |
| | | | GGGCTGGGCTTCGACGCCCTCGGACTGCTGCTCGCCCT | | |
| | | | CGGCGCCGCTTGCTGCCAGGTCGGCTACTTCGTCCTGT | | |
| | | | CCGACCAGGGCAGCGACGCCGGCGAGGAGGCGCCCGAC | | |
| | | | CCGCTCGGCGTCATCGCCTACGGCCTGCTGGTCGGCGC | | |
| | | | CGCCGTGCTCACCATCGTCGCCCGGCCCTGGTCGATGG | | |
| | | | ACTGGTCCGTCCTCGCCGGCTCGGCACCCATGGACGGC | | |
| | | | ACACCCGTCGCCGCCGCCCTGCTGCTGGCCTGGATCGT | | |
| | | | GCTCATCGCCACGGTGCTCGCCTACGTCACCGGAATCG | | |
| | | | TGGCCGTACGTCGGCTGTCGCCGCAGGTCGCCGGAGTC | | |
| | | | GTGGCGTGCCTGGAAGCGGTCATCGCGACGGTCCTGGC | | |
| | | | GTGGGTGCTGCTGGGCGAGCACCTCTCCGCCCCGCAGG | | |
| | | | TCGTCGGCGGCATCGTGGTGCTGGCGGGCGCCTTCATC | | |
| | | | GCCCAGTCCTCGACCCCGGCGAAGGGCTCCGCGGACCC | | |
| | | | GGTGGCCAGGGGCGGTCCCGAAAGGGAGTTGTCGAGCC | | |
| | | | GGGGAACGTCGACCTAG | | |
| putative | regulatory | AF265211 | GTGAAATTAAAAGATTTCGCTTTTTACGCCCCCTGTGT | 186 | |
| mem-brane | protein PecM | | CTGGGGAACCACCTACTTTGTCACCACCCAATTTCTGC | | |
| protein | [Pectobacterium | | CTGCCGACAAACCGCTGTTGGCTGCCCTGATCCGGGCG | | |
| NCgl0580 | | | TTGCCTGCTGGTATTATTCTCATTCTCGGTAAAACTCT | | |
| related | chrysanthemi] | | GCCGCCGGTCGGCTGGCTGTGGCGCTTGTTTGTACTGG | | |
| | | | GCGCACTCAATATCGGCGTGTTCTTTGTGATGCTGTTT | | |
| | | | TTTGCTGCTTATCGCCTGCCTGGCGGCGTGGTGGCGCT | | |
| | | | GGTGGGGTCGCTTCAGCCGCTGATCGTCATCCTGTTGT | | |
| | | | CTTTCCTGTTGCTGACGCAGCCGGTGCTGAAAAAGCAG | | |
| | | | ATGGTGGCGGCCGTGGCCGGCGGCATCGGTATTGCGTT | | |
| | | | GCTGATTTCGCTGCCGAAAGCGCCGCTGAACCCCGCCG | | |
| | | | GGCTGGTGGCATCGGCATTGGCGACGGTGAGTATGGCG | | |
| | | | TCCGGTCTGGTGCTGACTAAAAAGTGGGGGCGCCCGGC | | |
| | | | CGGAATGACGATGCTGACGTTTACCGGCTGGCAGCTGT | | |
| | | | TTTGCGGCGGGCTGGTGATTCTGCCGGTGCAGATGCTG | | |
| | | | ACAGAGCCGTTGCCGGATGTGGTGACCCTGACCAACCT | | |
| | | | TGCCGGTTATTTTTACCTGGCGATTCCCGGCTCTTTAC | | |
| | | | TGGCGTATTTCATGTGGTTCTCCGGTATTGAAGCTAAT | | |
| | | | TCGCCGGTGATGATGTCGATGCTGGGTTTTCTCAGCCC | | |
| | | | GTTGGTCGCGCTGTTTCTGGGCTTTTTATTTCTTCAAC | | |
| | | | AAGGACTTTCCGGAGCACAATTGGTCGGAGTGGTATTC | | |
| | | | ATTTTCTCGGCGATTATTATTGTTCAGGATGTTTCGTT | | |
| | | | ATTTAGCAGAAGAAAAAAAGTGAAGCAGTTGGAGCAAT | | |
| | | | CTGACTGTGCTGTCAAATAA | | |
| putative | Lactobacillus | AL935255 | ATGAAGCGTTTAGTTGGAACTCTGTGCGGTATTATTAG | 187 | |
| mem-brane | plantarum | | TGCCGCTTTATTTGGGCTAGGTGGAATACTAGCACAGC | | |
| protein | | | CTTTGTTAAGTGAGCAAGTTCTGACTCCGCAACAGATT | | |
| NCgl0580 | | | GTATTGTTACGGCTGTTAATCGGTGGGGCAATGTTGTT | | |
| related | | | GCTATATCGTAACTTGTTTTTCAAGCAGGCTAGAAAAA | | |
| | | | | | GCACGAAAAAGATTTGGACACATTGGCGAATTTTAACA |
| | | | CGAATTATGATATACGGCATCGCCGGCTTGTGCACGGC | | |
| | | | ACAAATTGCCTTTTTTTCTGCGATTAATTACAGTAATG | | |
| | | | CAGCAGTTGCAACTGTTTTTCAGTCCACTAGTCCGTTT | | |
| | | | ATTCTGCTTGTATTTACCGCGCTGAAAGCGAAAAGACT | | |
| | | | TCCCAGTTTATTAGCAGGAATGAGCTTAATAAGCGCAT | | |
| | | | TGATGGGAATCTGGCTTATTGTTGAATCCGGATTTAAG | | |
| | | | ACCGGATTAATAAAACCGGAAGCAATTATTTTTGGCCT | | |
| | | | GATTGCGGCTATCGGGGTTATCTTATACACCAAACTAC | | |
| | | | CTGTTCCATTGTTAAACCAAATTGCCGCAGTGGATATT | | |
| | | | TTGGGATGGGCACTAGTTATTGGCGGTGTGATAGCGTT | | |
| | | | GATTCACACACCGTTACCAAATTTAGTTAGATTTTCAA | | |
| | | | AAACGCAGCTTTTAGCGGTTCTTATCATTGTTATTCTA | | |
| | | | GCCACCGTTGTTGCGTATGATCTTTATTTAGAAAGTTT | | |
| | | | AAAGCTAATAGACGGATTTCTGGCAACTATGACTGGAC | | |
| | | | TATTTGAACCAATCAGTTCCGTACTTTTTGGCATGTTA | | |
| | | | TTCTTGCACCAAATCTTGGTTCCTCAGGCCTTGGTTGG | | |
| | | | TATTATATTGGTTGTGGGTGCAATTATGATACTGAATT | | |
| | | | TACCTCACCATATCACGGCACCTGTTCCCAGCAAAACC | | |
| | | | TGTCAATGTACGATGTCTAATCAATAG | | |
| putative | Lactobacillus | AL935252 | GTGAAGAAAATTGCGCCCCTGTTCGTTGGCTTAGGGGC | 188 | |
| mem-brane | plantarum | | CATTAGTTTTGGAATTCCGGCGTCACTATTTAAAATTG | | |
| protein | | | CGCGTCGGCAGGGGGTTGTCAATGGCCCATTGCTATTC | | |
| NCgl0580 | | | TGGTCCTTTCTGAGTGCGGTTGTGATTTTAGGTGTGAT | | |
| related | | | TCAAATTTTACGCCGTGCACGTTTGCGTAATCAGCAAA | | |
| | | | CGAATTGGAAGCAAATCGGACTGGTAATTGCGGCTGGA | | |
| | | | ACGGCTTCGGGATTTACTAACACCTTTTACATACAGGC | | |
| | | | GTTAAAGCTTATCCCAGTTGCTGTGGCCGCGGTAATGT | | |
| | | | TGATGCAGGCGGTCTGGATATCAACATTACTAGGAGCA | | |
| | | | GTGATTCATCATCGGCGTCCCTCCCGACTGCAAGTGGT | | |
| | | | TAGCATTGTTTTGGTATTGATAGGCACGATTTTAGCTG | | |
| | | | CTGGTCTGTTTCCAATTACGCAGGCGCTCTCGCCGTGG | | |
| | | | GGCTTGATGTTAAGTTTTTTAGCGGCATGCTCGTATGC | | |
| | | | TTGCACGATGCAGTTTACGGCTAGCTTAGGCAATAACT | | |
| | | | TAGACCCGTTATCGAAAACATGGTTACTGTGTTTGGGC | | |
| | | | GCTTTCATACTCATTGCTATCGTGTGGTCACCGCAATT | | |
| | | | AGTTACGGCACCCACCACGCCAGCAACAGTCGGCTGGG | | |
| | | | GAGTACTGATTGCACTATTCTCAATGGTTTTCCCACTG | | |
| | | | GTTATGTATTCATTGTTTATGCCGTACTTAGAGCTTGG | | |
| | | | CATTGGCCCAATCCTTTCTTCTTTAGAATTACCAGCCT | | |
| | | | CGATTGTTGTTGCATTTGTACTGCTTGATGAAACTATT | | |
| | | | GATTGGGTGCAAATGGTTGGCGTGGCCATTATTATTAC | | |
| | | | GGCCGTAATTCTGCCAAACGTGTTAAATATGCGACGAG | | |
| | | | TTCGGCCATAG | | |
| putative | Lactobacillus | AL935261 | ATGACAACTAACCGTTATATGAAGGGCATCATGTGGGC | 189 | |
| mem-brane | plantarum | | GATGTTGGCCTCGACCCTGTGGGGAGTCTCAGGTACAG | | |
| protein | | | TGATGCAGTTCGTATCACAAAACCAAGCCATCCCGGCT | | |
| NCgl0580 | | | GATTGGTTCTTATCTGTAAGGACGTTATCTGCTGGAAT | | |
| related | | | CATTCTGTTAGCGATTGGATTTGTGCAACAGGGTACCA | | |
| | | | AAATCTTCAAAGTCTTTAGATCTTGGGCGTCGGTTGGA | | |
| | | | CAATTAGTGGCATACGCGACAGTGGGATTGATGGCGAA | | |
| | | | TATGTATACTTTTTACATCAGTATTGAGCGCGGAACAG | | |
| | | | CCGCTGCCGCCACTATTTTACAATACTTAAGTCCTTTG | | |
| | | | TTTATTGTACTAGGAACGTTGCTGTTTAAACGGGAACT | | |
| | | | GCCTTTACGGACTGATTTAATTGCGTTTGCGGTCTCCT | | |
| | | | TGTTGGGGGTGTTTTTAGCAATCACTAAGGGTAATATT | | |
| | | | CATGAGTTGGCGATTCCGATGGATGCACTCGTCTGGGG | | |
| | | | AATCCTTTCGGGGGTAACAGCGGCCTTGTACGTAGTCT | | |
| | | | TGCCGCGAAAGATTGTAGCCGAAAATTCACCGGTCGTG | | |
| | | | ATTCTTGGTTGGGGGACATTGATTGCGGGAATCCTATT | | |
| | | | TAATTTATATCACCCAATTTGGATCGGTGCACCAAAAA | | |
| | | | TTACACCAACGCTAGTGACTTCAATTGGCGCCATCGTT | | |
| | | | TTAATCGGGACGATTTTTGCTTTCTTATCGTTGCTACA | | |
| | | | TAGTCTACAGTACGCGCCGTCTGCGGTGGTCAGTATTG | | |
| | | | TTGATGCCGTCCAACCAGTAGTGACTTTTGTACTAAGT | | |
| | | | ATTATTTTCTTAGGCTTACAAGTGACATGGGTCGAAAT | | |
| | | | CCTCGGCTCGTTATTGGTGATTGTCGCGATTTATATCT | | |
| | | | TGCAGCAGTATCGGAGTGATCCGGCTAGTGATTAG | | |
| NCgl0580 | Coryne- | NC_003450 | ATGAATAAACAGTCCGCTGCAGTGTTGATGGTGATGGG | 277 | |
| | bacterium | | TTCCGCCCTATCCCTGCAATTTGGTGCTGCCATTGGAA | | |
| | glutamicum | | CGCAGCTTTTCCCCCTCAACGGCCCCTGGGCTGTCACC | | |
| | | | TCTTTAAGGCTGTTCATCGCAGGCTTGATCATGTGCCT | | |
| | | | GGTGATCCGCCCGCGACTTCGTTCCTGGACTAAAAAAC | | |
| | | | AATGGATCGCCGTGCTGCTGTTGGGATTATCTCTTGGC | | |
| | | | GGAATGAACAGCCTGTTTTACGCATCCATCGAACTCAT | | |
| | | | CCCGCTGGGTACCGCCGTGACCATTGAGTTCCTCGGCC | | |
| | | | CCCTGATTTTCTCCGCGGTGTTAGCCCGCACGCTGAAA | | |
| | | | AACGGATTGTGCGTGGCTTTAGCGTTTCTCGGCATGGC | | |
| | | | ACTACTGGGTATCGATTCCCTCAGCGGCGAAACCCTTG | | |
| | | | ACCCACTCGGCGTCATTTTCGCAGCCGTCGCAGGAATC | | |
| | | | TTCTGGGTGTGCTACATCCTGGCATCAAAGAAAATCGG | | |
| | | | CCAACTCATCCCCGGAACAAGCGGCCTGGCCGTCGCAC | | |
| | | | TGATTATCGGCGCAGTGGCAGTATTTCCACTGGGTGCT | | |
| | | | ACACACATGGGCCCGATTTTCCAGACCCCAACCCTACT | | |
| | | | CATCCTGGCGCTTGGCACAGCACTTCTCGGGTCGCTTA | | |
| | | | TCCCCTATTCGCTGGAATTATCGGCACTGCGCCGACTC | | |
| | | | CCCGCCCCCATTTTCAGTATTCTGCTCAGCCTCGAACC | | |
| | | | GGCATTCGCCGCCGCCGTCGGCTGGATCCTGCTTGATC | | |
| | | | AAACCCCCACCGCGCTCAAGTGGGCCGCGATCATCCTT | | |
| | | | GTCATCGCGGCCAGCATCGGCGTCACGTGGGAGCCTAA | | |
| | | | AAAGATGCTTGTCGACGCGCCCCTCCACTCAAAATGCA | | |
| | | | ACGCGAAGAGGCGAGTACACACACCTAGT | | |
| drug | Streptomyces | AL939108 | GTGTCGAATGCCGTCTCCGGCCTGCCCGTAGGGCGTGG | 190 | |
| permease | coelicolor | | CCTCCTCTATCTGATCGTCGCCGGTGTCGCCTGGGGCA | | |
| NCgl2065 | | | CCGCCGGTGCCGCCGCCTCGCTGGTCTACCGGGCCAGC | | |
| related | | | GACCTGGGGCCCGTCGCCCTGTCGTTCTGGCGTTGCGC | | |
| | | | GATGGGGCTCGTGCTGCTGCTCGCCGTCCGCCCGCTGC | | |
| | | | GCCCGCGGCTGCGCCCGCGGCTGCGCCCGCGGCTGCGC | | |
| | | | CCGGCGGTCCGCGAACCGTTCGCCCGCAGGACGCTTCG | | |
| | | | GGCCGGTGTCACCGGTGTCGGGCTCGCGGTGTTCCAGA | | |
| | | | CCGCCTACTTCGCCGCCGTGCAGTCCACCGGACTCGCC | | |
| | | | GTCGCCACGGTGGTCACCCTCGGCGCGGGGCCCGTACT | | |
| | | | GATCGCCCTCGGCGCGCGCCTCGCCCTCGGTGAACAGC | | |
| | | | TGGGAGCGGGGGGTGCCGCGGCCGTGGCCGGCGCCCTC | | |
| | | | GCCGGGCTCCTGGTGCTCGTCCTCGGCGGCGGAAGCGC | | |
| | | | GACCGTCCGCCTGCCGGGTGTGCTCCTCGCGCTGCTGT | | |
| | | | CCGCCGCCGGGTACTCGGTGATGACGCTGCTCACCCGT | | |
| | | | TGGTGGGGACGGGGCGGCGGGGCGGACGCGGCCGGTAC | | |
| | | | GTCCGTGGGGGCGTTCGCCGTCACGAGTCTGTGCCTGC | | |
| | | | TGCCGTTCGCCCTGGCCGAGGGCCTGGTGCCGCACACC | | |
| | | | GCGGAACCGGTCCGGCTGCTGTGGCTCCTCGCCTACGT | | |
| | | | CGCGGCCGTCCCGACCGCGCTGGCCTACGGGCTCTACT | | |
| | | | TCGCCGGCGCGGCCGTCGTCCGGTCCGCGACGGTCTCC | | |
| | | | GTGATCATGCTCCTGGAGCCGGTCAGTGCGGCCGCGCT | | |
| | | | CGCCGTCCTGCTGCTCGGCGAGCACCTCACGGCCGCGA | | |
| | | | CCCTGGCCGGCACGCTGCTGATGCTCGGCTCGGTCGCG | | |
| | | | GGTCTCGCGGTGGCGGAGACCCGGGCGGCGCGGGAGGc | | |
| | | | GAGGACGCGGCCGGCGCCCGCGTGA | | |
| drug | Streptomyces | AL939124 | GTGAACGTCCTGCTCTCGGCCGCCTTCGTTCTGTGCTG | 191 | |
| permease | coelicolor | | GAGCTCCGGCTTCATCGGCGCCAAGCTCGGTGCTCAGA | | |
| NCgl2065 | | | CCGCGGCCACACCCACCCTCCTGATGTGGCGCTTCCTG | | |
| related | | | CCTCTCGCCGTGGCCCTGGTCGCCGCGGCGGCCGTCTC | | |
| | | | CCGGGCCGCCTGGCGGGGCCTGACACCGCGGGACGCCG | | |
| | | | GCCGGCAGATCGCCATCGGCGCCCTGTCGCAGAGCGGC | | |
| | | | TATCTGCTCAGCGTCTACTACGCCATCGAACTGGGCGT | | |
| | | | CTCCAGCGGCACCACCGCCCTCATCGACGGCGTCCAGC | | |
| | | | CACTCGTCGCCGGCGCGCTCGCCGGTCCCCTGCTGCGC | | |
| | | | CAGTACGTCTCGCGCGGGCAGTGGCTCGGACTGTGGCT | | |
| | | | GGGCTGTCGGGCGTGGCCACCGTGACGGTCGCCGACG | | |
| | | | CCGGGGCGGCGGGCGCGGAGGTGGCCTGGTGGGCGTAT | | |
| | | | CTCGTCCCGTTTCTCGGCATGCTGTCGCTGGTGGCGGC | | |
| | | | CACCTTCCTGGAGGGCCGCACAAGGGTGCCGGTCGCGC | | |
| | | | CCCGCGTCGCCCTGACGATCCACTGTGCGACCAGTGCC | | |
| | | | GTCCTCTTCTCCGGACTGGCCCTGGGCCTCGGGGCGGC | | |
| | | | GGCACCGCCGGCCGGTTCCTCGTTCTGGCTGGCGACCG | | |
| | | | CCTGGCTGGTGGTCCTGCCGACCTTCGGCGGCTACGGC | | |
| | | | CTGTACTGGCTGATCCTGCGCCGGTCCGGCATCACCGA | | |
| | | | GGTCAACACCCTCATGTTCCTCATGGCCCCGGTCACGG | | |
| | | | CCGTGTGGGGCGCCCTCATGTTCGGTGAGCCGTTCGGC | | |
| | | | GTCCAGACCGCCCTCGGCCTGGCGGTCGGCCTCGCGGC | | |
| | | | CGTGGTCGTCGTCCGGCGCGGGGGCGGCGCGCGCCGGG | | |
| | | | AGCGGCCCGTGCGGTCCGGCGCGGACCGTCCGGCGGCC | | |
| | | | GGAGGGCCGACGGCGGACCAGCCGACGAACAGGCCGAC | | |
| | | | CGACAGGCCGACGGCGGCCGGGTCGACCGACAGGCCGA | | |
| | | | CGGCGGACAGGCGCTGA | | |
| drug | Thermobifida | NZ_AAAQ010 | ATGTCTGATTTCCGCAAGGGTGTGCTCTATGGCGCCAG | 192 | |
| permease | fusca | 00034 | TTCGTACTTCATGTGGGGCTTTCTGCCGCTCTACTGGC | | |
| NCgI2065 | | | CGCTGCTGACCCCGCCTGCCACGGCCTTTGAGGTCCTC | | |
| related | | | TTACATAGGATGATCTGGTCATTGGTTGTCACGCTCGT | | |
| | | | GGTGCTGCTGGTGCAGCGGAACTGGCAGTGGATCCGCG | | |
| | | | GCGTGCTGCGGAGCCCGCGGCGCCTGCTGCTGCTCCTC | | |
| | | | GCCTCGGCCGCACTCATCTCCCTGAACTGGGGCGCTTT | | |
| | | | CATCACCGCCGTGACGACCGGGCACACCCTGCAATCGG | | |
| | | | CACTCGCCTACTTCATCAACCCGCTGGTGAGCGTGGCG | | |
| | | | CTAGGGCTGCTGGTGTTCAAAGAGCGGCTGCGCCCAGG | | |
| | | | CCAGTGGGCCGCACTGCTGCTCGGCGTCCTCGCCGTAG | | |
| | | | CCGTGCTGACCGTCGACTACGGCTCCCTGCCTTGGTTG | | |
| | | | GCGCTGGCCATGGCGTTCTCCTTCGCCGTCTACGGCGC | | |
| | | | GCTGAAGAAGTTCGTGGGCTTGGACGGGGTGGAGAGCC | | |
| | | | TCAGCGCGGAGACCGCGGTCCTGTTCCTGCCTGCGCTG | | |
| | | | GGCGGCGCGGTCTACCTGGAAGTGACCGGTACCGGCAC | | |
| | | | CTTCACCTCGGTCTCCCCCCTCCACGCGTTGCTGCTGG | | |
| | | | TGGGCGCCGGAGTGGTGACCGCGGCGCCGCTCATGCTG | | |
| | | | TTCGGCGCGGCAGCGCACCGCATCCCGCTGACCCTGGT | | |
| | | | CGGGCTGCTGCAGTTCATGGTTCCGGTGATGCACTTCC | | |
| | | | TCATCGCCTGGCTGGTCTTCGGGGAGGACCTGTCACTT | | |
| | | | GGCCGGTGGATCGGGTTCGCCGTGGTGTGGACCGCGCT | | |
| | | | CGTGGTGTTCGTCGTCGACATGCTCCGCCACGCACGCC | | |
| | | | ACACCCCCCGCCCTGCCCCGTCAGCCCCTGTCGCTGAG | | |
| | | | GAAGCCGAGGAAACTGCGGCTAGTTGA | | |
| drug | Streptomyces | AL939120 | GTGGCCGGGTCGTCCAGGAGTGATCAGCGAGTAGGCCT | 193 | |
| permease | coelicolor | | GCTGAACGGCTTCGCGGCGTACGGGATGTGGGGGCTCG | | |
| NCgl2065 | | | TCCCGCTGTTCTGGCCGCTGCTCAAGCCCGCCGGGGCC | | |
| related | | | GGGGAGATCCTCGCCCACCGGATGGTGTGGTCCCTCGC | | |
| | | | CTTCGTCGCCGTCGCCCTCCTCTTCGTACGGCGCTGGG | | |
| | | | CCTGGGCCGGCGAGCTGCTGCGGCAGCCGCGCAGGCTC | | |
| | | | GCCCTGGTCGCGGTGGCCGCCGCGGTCATCACCGTCAA | | |
| | | | CTGGGGCGTCTACATCTGGGCCGTGAACAGCGGCCATG | | |
| | | | TCGTCGAGGCCTCGCTCGGCTACTTCATCAACCCGCTG | | |
| | | | GTCACCATCGCGATGGGCGTGCTGTTGCTCAAGGAGCG | | |
| | | | GCTGCGGCCCGCGCAGTGGGCGGCGGTCGGCACCGGCT | | |
| | | | TCGCGGCCGTGCTCGTGCTCGCCGTCGGCTACGGCCAG | | |
| | | | CCGCCGTGGATCTCGCTCTGCCTCGCCTTCTCCTTCGC | | |
| | | | CACGTACGGCCTGGTGAAGAAGAAGGTCAACCTCGGGG | | |
| | | | GTGTCGAGTCGCTGGCCGCCGAGACGGCGATCCAGTTC | | |
| | | | CTTCCGGCGCTCGGCTACCTGCTGTGGCTGGGCGCGCA | | |
| | | | GGGCGAGTCGACCTTCACCACGGAGGGCGCCGGACACT | | |
| | | | CGGCCCTGCTCGCCGCGACCGGCGTCGTCACGGCGATC | | |
| | | | CCGCTGGTCTGCTTCGGCGCGGCGGCGATCCGCGTCCC | | |
| | | | GCTGTCCACACTGGGGCTGCTGCAATACCTGGCGCCGG | | |
| | | | TCTTCCAGTTCCTGCTCGGCGTCCTCTACTTCGGCGAG | | |
| | | | GCCATGCCGCCCGAGCGCTGGGCCGGCTTCGGGCTGGT | | |
| | | | CTGGCTGGCGCTGACGCTGCTCACCTGGGACGCGTTGC | | |
| | | | GCACGGCCCGCCGGACCGCACGGGCGCTGAGGGAACAA | | |
| | | | CTGGACCGGTCGGGCGCGGGCGTACCACCGCTCAAGGG | | |
| | | | GGCCGCCGCCGCGCGGGAGCCGAGGGTCGTGGCCTCGG | | |
| | | | GGACTCCGGCACCGGGCGCCGGCGACGCACCGCAGCAA | | |
| | | | CAGCAACAGCAACAGCAACAGCAACAGCAACAGCAACA | | |
| | | | CGGAACCAGGGCCGGGAAGCCGTAG | | |
| drug | Lactobacillus | AL935253 | GTGAAGAAAGCATATCTTTACATTGCAATTTCGACCTT | 194 | |
| permease | plantarum | | AATGTTTAGTTCGATGGAAATTGCGCTAAAGATGGCCG | | |
| NCgl2065 | | | GCAGTGCCTTTAACCCAATCCAATTGAATCTAATTCGA | | |
| related | | | TTTTTTATTGGGGCAATTGTGTTACTGCCATTTGCATT | | |
| | | | GCGGGCATTAAAGCAAACCGGACGAAAGTTAGTGAGTG | | |
| | | | CTGACTGGCGGCTATTTGCTTTAACCGGGCTAGTGTGT | | |
| | | | GTCATTGTCAGTATGTCGCTTTACCAACTCGCGATTAC | | |
| | | | GGTCGATCAAGCTTCGACTGTGGCCGTATTGTTTAGTT | | |
| | | | GTAATCCGGTATTTGCGCTATTATTCTCCTATTTAATT | | |
| | | | CTGCGAGAACGGTTGGGTCGAGCTAACTTGATCTCCGT | | |
| | | | CGTGATTTCTGTGATTGGGTTGTTGATCATTGTTAATC | | |
| | | | CGGCCCATTTGACGAATGGGCTCGGGCTGCTATTAGCC | | |
| | | | ATCGGGTCTGCCGTGACTTTTGGGCTGTACAGTATCAT | | |
| | | | CTCGCGTTATGGGTCTGTTAAACGGGGCTTGAATGGGC | | |
| | | | TGACGATGACTTGTTTTACTTTCTTTGCTGGTGCGTTT | | |
| | | | GAACTTCTAGTTTTAGCTTGGATTACTAAGATTCCGGC | | |
| | | | TGTCGCCAATGGGTTGACGGCCATCGGTTTGCGGCAAT | | |
| | | | TTGCTGCCATTCCGGTTTTGGTGAATGTTAATCTCAAC | | |
| | | | TATTTCTGGTTACTATTTTTTATCGGCGTTTGTGTTAC | | |
| | | | TGGTGGGGGCTTCGCGTTTTATTTCTTGGCAATGGAAC | | |
| | | | AAACCGATGTTTCAACGGCTTCCCTAGTATTCTTCATT | | |
| | | | AAGCCGGGGTTGGCGCCAATCTTAGCAGCGTTGATCCT | | |
| | | | CCATGAACAAATTTTGTGGACGACAGTGGTCGGAATTG | | |
| | | | TTGTGATTTTGATTGGTTCCGTCGTGACCTTTGTCGGT | | |
| | | | AATCGGTTCCGTGAACGGGATACGATGGGTGCGATTGA | | |
| | | | GCAGCCAACAGCGGCCGCCACTGATGATGAACATGTCA | | |
| | | | TCAAAGCCGCACACGCCGTTTCAAATCAAGAAAATTAA | | |
| NCgl2065 | Coryne- | NC_003450 | GTGAATGATGCTGGCTTGAAGACGCGAAACCCGGTGcT | 278 | |
| | bacterium | | TGCCCCCATTTTGATGGTGGTTAACGGCGTGTCCCTTT | | |
| | glutamicum | | ATGCCGGAGCAGCGTTGGCGGTGGGGCTGTTTGAGAGT | | |
| | | | TTCCCACCCGCGTTGGTTGCGTGGATGCGAGTAGCAGC | | |
| | | | GGCTGCGGTGATTTTGCTTGTGCTGTATCGGCCTGCAG | | |
| | | | TGCGAAATTTTATTGGGCAGACCGGGTTTTATGCGGCG | | |
| | | | GTGTATGGCGTTTCCACGCTTGCCATGAACATCACGTT | | |
| | | | CTATGAGGCGATCGCCCGCATTCCGATGGGTACCGCGG | | |
| | | | TGGCCATTGAGTTCTTGGGACCTATTGCAGTGGCCGCG | | |
| | | | TTGGGCAGTAAGACGCTGCGGGATTGGGCTGCGTTGGT | | |
| | | | TTTAGCTGGCATCGGAGTGATAATTATTAGCGGTGCGC | | |
| | | | AGTGGTCGGCCAACAGCGTGGGCGTCATGTTTGCACTG | | |
| | | | GCAGCAGCATTACTGTGGGCTGCGTACATCATCGCGGG | | |
| | | | AAACCGCATTGCAGGCGATGCCTCCTCAAGTAGAACCG | | |
| | | | GCATGGCGGTGGGATTCACGTGGGCATCAGTGTTGTCT | | |
| | | | TTGCCGTTGGCGATCTGGTGGTGGCCGGGTCTGGGAGC | | |
| | | | AACGGAACTTACGTTAATCGAGGTCATCGGATTAGCAC | | |
| | | | TTGGTTTGGGCGTGCTGTCGGCGGTGATTCCTTATGGC | | |
| | | | CTTGACCAGATTGTGCTCCGCATGGCCGGGCGATCCTA | | |
| | | | CTTTGCGCTGCTCCTGGCTATTTTGCCGATCAGCGCCG | | |
| | | | CGCTCATGGGAGCGCTTGCGCTGGGCCAAATGTTGTCG | | |
| | | | GTGGCTGAGCTTGTCGGCATTGTGCTGGTTGTCATCGC | | |
| | | | AGTTGCTTTGCGACGCCCCTCC | | |
| hypo-thetical | Thermobifida | NZ_AAAQ010 | GTGAACGCCGACACCCTCCTGTGGTCCCTGCTGCTCGG | 195 | |
| mem-brane | fusca | 00035 | CGTCATCGTCGTCGCTGCCGCGGCGGCGATCATCATCC | | |
| protein | | | CCACCGTGCGGAACAGCAGCACGGCTCCCCCGCCCGGG | | |
| NCgl2829 | | | GCGGTAGGGACCGCGCTGGGTGCGGCGCTCACCGCCGC | | |
| related | | | TGCCCTCGGCATAGCGGGCAGCGGAACCGCTCCCGCCT | | |
| | | | CCGAAGTGCCCGCGGGCTCCGGCCAGGTCCGTACCGTC | | |
| | | | GACGTGGTGCTGGGCGACATGACCGTCTCCCCGTCCCA | | |
| | | | CGTCACCGTCGCGCCCGGCGACTCCCTCGTCCTCCGCG | | |
| | | | TGCGCAACGAGGACACTCAAGTCCACGACTTGGTGGTG | | |
| | | | GAGACCGGGGCCCGCACGCCCCGGCTTGCGCCAGGTGA | | |
| | | | CAGCGCCACCCTGCAGGTCGGCACGGTGACCGAGCCCA | | |
| | | | TCGACGCCTGGTGCACTGTGCTCGGGCACAGCGCCGCG | | |
| | | | GGCATGCGGATGCGGATCGACACCACTGACACTGCGGA | | |
| | | | CAGCGCTGACAGCCCCGACACGCCCGCTGGTGCGGACA | | |
| | | | GCGGTCCGCCCGCACCGCTCCCCCTGTCCGCGGAGATG | | |
| | | | AGCGACGACTGGCAGCCCCGCGACGCTGTCCTGCCGCC | | |
| | | | CGCGCCGGACCGCACCGAACACGAAGTGGAGATCCGGG | | |
| | | | TCACCGAAACCGAGCTGGAGGTCGCCCCCGGGGTGCGG | | |
| | | | CAGAGCGTGTGGACGTTCGGCGGCGACGTCCCCGGCCC | | |
| | | | TGTGCTGCGCGGCAAGGTCGGCGACGTGTTCACCGTGA | | |
| | | | CCTTCGTCAACGACGGCACGATGGGCCACGGCATCGAC | | |
| | | | TTCCACGCCAGCAGTCTCGCCCCGGACGAGCCGATGCG | | |
| | | | CACGATCAATCCGGGCGAGCGCCTCACCTACCGGTTCC | | |
| | | | GCGCGGAGAAAGCCGGTGCCTGGGTGTACCACTGTTCG | | |
| | | | ACCTCGCCCATGCTGCAGCACATCGGCAACGGCATGTA | | |
| | | | CGGCGCGGTCATCATCGACCCGCCCGACCTTGAGCCGG | | |
| | | | TCGACCGTGAATACCTGCTGGTCCAAGGAGAGCTGTAC | | |
| | | | CTGGGCGAGCCGGGCAGCGCCGACCAGGTCGCCCGGAT | | |
| | | | GCGGGCGGGTGAGCCGGACGCGTGGGTGTTCAACGGGG | | |
| | | | TCGCCGCCGGCTACGCCCACGCGCCGTTGACCGCCGAG | | |
| | | | GTCGGGGAGCGCGTCCGGATCTGGGTGGTGGCGGCCGG | | |
| | | | TCCCACCAGCGGAACGTCTTTCCACATCGTCGGCGCCC | | |
| | | | AGTTCGACACCGTCTACAAGGAGGGTGCCTACCTGGTG | | |
| | | | CGCCGTGGCGACGCCGGGGGCGCGCAAGCGCTCGACCT | | |
| | | | GGCGGTCGCCCAAGGCGGTTTTGTCGAAACAGTGTTCC | | |
| | | | CCGAAGCGGGCTCCTATCCCTTTGTCGACCATGACATG | | |
| | | | CGGCATGCCGAGAACGGGGCCCGCGGCTTCTTCACGAT | | |
| | | | CACGGAGTGA | | |
| NCgl2829 | Coryne- | NC_003450 | ATGGTTCTGGTAATCGCCGGAATAATCCACCCGCTCCT | 279 | |
| | bacterium | | GCCGGAATACCGTTGGGTTCTCATTCACCTTTTCACCC | | |
| | glutamicum | | TTGGTGCCATCACCAATTCGATTGTGGTGTGGTCGCAG | | |
| | | | CATTTCACGGAAAAGTTTCTGCATTTAAAGCTTGAGGA | | |
| | | | ATCGAAACGCCCTGCGCAGCTACTGAAAATTCGGGTGC | | |
| | | | TGAATGTGGGAATTATCGTCACGATTATTGGGCAGATG | | |
| | | | ATCGGTCAGTGGATCGTCACCAGTGTCGGCGCGACGAT | | |
| | | | TGTGGGCGGTGCTTTGGCGTGGCACGCAGGCAGTTTGG | | |
| | | | CATCACAGTTCCGGAGCGCAAAACGCGGTCAGCCTTTC | | |
| | | | GCGTCGGCAGTGATCGCGTATGTTGCCAGCGCGTGCTG | | |
| | | | CCTGCCGTTTGGCGCATTTGCCGGAGCGTTGTTGTCCA | | |
| | | | AGGAGCTGTCGGGACATCTCCAGGAACGAGTCCTTCTC | | |
| | | | ACCCACACGGTGATTAATTTTCTAGGTTTCGTGGGATT | | |
| | | | TGCTGCGCTCGGTTCGCTGTCGGTGCTGTTCGCCGCGA | | |
| | | | TTTGGCGCACCAAAATTCGCCACAATTTCACCCCGTGG | | |
| | | | TCTGTGGGGATCATGGCGGTGAGCCTGCCGATCATCGT | | |
| | | | CACGGGCATCCTGCTCAACAACGGCTATGTCGCCGCCA | | |
| | | | CAGGCCTGGCCGCGTACGTGGCAGCATGGTTGCTGGCC | | |
| | | | ATGGTGGGGTGGGGGAAGGCGTCGATAAGCAATTTAAG | | |
| | | | CTTTTCGACGTCCACCTCCACCACCGCACCCCTTTGGC | | |
| | | | TCGTGGGCACGCTTGTGTGGCTGGCGGTGCAGGCGGTG | | |
| | | | ATGCATGACGGCGAGCTTTACCATGTGGAAGTTCCCAC | | |
| | | | GATTGCGCTGGTCATCGGCTTTGGCGCGCAGCTTCTGA | | |
| | | | TCGGTGTGATGAGTTATCTACTGCCGTCGACGATGGGT | | |
| | | | GGCGGCGCGAGCGCGGTGCGGACTGGAACGCACATTTT | | |
| | | | AAACACTGCGGGGCTGTTTAGGTGGACGCTGATCAACG | | |
| | | | GTGGCCTGGCGATTTGGCTGCTCACCGACAATTCGTGG | | |
| | | | CTGCGCGTCGTGGTGTCTCTGCTGAGTATCGGAGCGTT | | |
| | | | GGCAGTTTTTGTCATTCTGCTGCCCAAGGCTGTGCGGG | | |
| | | | CGCAGCGCGGAGTGATCACCAAAAAGCGCGAACCAATT | | |
| | | | ACTCCGCCGGAGGAGCCTCGACTCAATCAAATTACCGC | | |
| | | | GGGAATCTCTGTGCTTGCCCTGATTTTGGCAGCATTCG | | |
| | | | GTGGGCTCAACCCCGGTGTTGCGCCGGTGGCAAGCTCA | | |
| | | | AATGAAGACGTCTATGCTGTGACCATTACCGCAGGTGA | | |
| | | | CATGGTGTTTATCCCTGATGTGATTGAAGTGCCTGCTG | | |
| | | | GTAAATCACTCGAAGTCACGATGCTCAACGAAGACGAC | | |
| | | | ATGGTGCACGATCTGAAATTTGCCAACGGTGTGCAAAC | | |
| | | | CGGACGTGTGGCGCCAGGTGATGAAATTACGGTGACCG | | |
| | | | TCGGCGATATTTCCGAAGACATGGACGGCTGGTGCACC | | |
| | | | ATCGCTGGGCACCGCGCGCAAGGAATGGATCTGGAAGT | | |
| | | | AAAGGTTGCGGCTCCGAAT | | |
| yggA | Escherichia coli | U28377 | GTGTTTTCTTATTACTTTCAAGGTCTTGCACTTGGGGC | 280 | |
| | | | GGCTATGATCCTACCGCTCGGTCCACAAAATGCTTTTG | | |
| | | | TGATGAATCAGGGCATACGTCGTCAGTACCACATTATG | | |
| | | | ATTGCCTTACTTTGTGCTATCAGCGATTTGGTCCTGAT | | |
| | | | TTGCGCCGGGATTTTTGGTGGCAGCGCGTTATTGATGC | | |
| | | | AGTCGCCGTGGTTGCTGGCGCTGGTCACCTGGGGCGGC | | |
| | | | GTAGCCTTCTTGCTGTGGTATGGTTTTGGCGCTTTTAA | | |
| | | | AACAGCAATGAGCAGTAATATTGAGTTAGCCAGCGCCG | | |
| | | | AAGTCATGAAGCAAGGCAGATGGAAAATTATCGCCACC | | |
| | | | ATGTTGGCAGTGACCTGGCTGAATCCGCATGTTTACCT | | |
| | | | GGATACTTTTGTTGTACTGGGCAGCCTTGGCGGGCAAC | | |
| | | | TTGATGTGGAACCAAAACGCTGGTTTGCACTCGGGACA | | |
| | | | ATTAGCGCCTCTTTCCTGTGGTTCTTTGGTCTGGCTCT | | |
| | | | TCTCGCAGCCTGGCTGGCACCGCGTCTGCGCACGGCAA | | |
| | | | AAGCACAGCGCATTATCAATCTGGTTGTGGGATGTGTT | | |
| | | | ATGTGGTTTATTGCCTTGCAGCTGGCGAGAGACGGTAT | | |
| | | | TGCTCATGCACAAGCCTTGTTCAGT | | |

A number of embodiments of the invention have been described. Nevertheless, it will be understood that various modifications may be made without departing from the spirit and scope of the invention. Accordingly, other embodiments are within the scope of the following claims.

1. An Enterobacteriaceae or coryneform bacterium comprising at least one of:
(a) a nucleic acid molecule comprising a sequence encoding a heterologous bacterial aspartokinase polypeptide or a functional variant thereof;
(b) a nucleic acid molecule comprising a sequence encoding a heterologous bacterial aspartate semialdehyde dehydrogenase polypeptide or a functional variant thereof;
(c) a nucleic acid molecule comprising a sequence encoding a heterologous bacterial phosphoenolpyruvate carboxylase polypeptide or a functional variant thereof;
(d) a nucleic acid molecule comprising a sequence encoding a heterologous bacterial pyruvate carboxylase polypeptide or a functional variant thereof;
(e) a nucleic acid molecule comprising a sequence encoding a heterologous bacterial dihydrodipicolinate synthase polypeptide or a functional variant thereof;
(f) a nucleic acid molecule comprising a sequence encoding a heterologous bacterial homoserine dehydrogenase polypeptide or a functional variant thereof;
(g) a nucleic acid molecule comprising a sequence encoding a heterologous bacterial homoserine O-acetyltransferase polypeptide or a functional variant thereof;
(h) a nucleic acid molecule comprising a sequence encoding a heterologous bacterial O-acetylhomoserine sulfhydrylase polypeptide or a functional variant thereof;
(i) a nucleic acid molecule comprising a sequence encoding a heterologous bacterial methionine adenosyltransferase polypeptide or a functional variant thereof;
(j) a nucleic acid molecule comprising a sequence encoding a heterologous bacterial mcbR gene product polypeptide or a functional variant thereof;
(k) a nucleic acid molecule comprising a sequence encoding a heterologous bacterial O-succinylhomoserine/acetylhomoserine (thiol)-lyase polypeptide or a functional variant thereof;
(l) a nucleic acid molecule comprising a sequence encoding a heterologous bacterial cystathionine beta-lyase polypeptide or a functional variant thereof;
(m) a nucleic acid molecule comprising a sequence encoding a heterologous bacterial 5-methyltetrahydrofolate homocysteine methyltransferase polypeptide or a functional variant thereof; and
(n) a nucleic acid molecule comprising a sequence encoding a heterologous bacterial 5-methyltetrahydropteroyltriglutamate-homocysteine methyltransferase polypeptide or a functional variant thereof.

2. The bacterium of claim 1, wherein the bacterium is an Escherichia coli bacterium.

3. The bacterium of claim 1, wherein the bacterium is a Corynebacterium glutamicum bacterium.

4. The bacterium of claim 1, wherein the sequence encodes a polypeptide with reduced feedback inhibition.

5. The bacterium of claim 1, wherein the polypeptide is selected from an Enterobacteriaceae polypeptide, an Actinomycetes polypeptide, or a variant thereof.

6. The bacterium of claim 5, wherein the polypeptide is a polypeptide of one of the following Actinomycetes species: Mycobacterium smegmatis, Streptomyces coelicolor, Thermobifida fusca, Amycolatopsis mediterranei and coryneform bacteria, including Corynebacterium glutamicum.

7. The bacterium of claim 5, wherein the polypeptide is a polypeptide of one of the following Enterobacteriaceae species: Erwinia chysanthemi and Escherichia coli.

8. The bacterium of claim 1, wherein the heterologous bacterial aspartokinase polypeptide or functional variant thereof is chosen from:
(a) a Mycobacterium smegmatis aspartokinase polypeptide or a functional variant thereof;
(b) an Amycolatopsis mediterranei aspartokinase polypeptide or a functional variant thereof;
(c) a Streptomyces coelicolor aspartokinase polypeptide or a functional variant thereof;
(d) a Thermobifida fusca aspartokinase polypeptide or a functional variant thereof;
(e) an Erwinia chrysanthemi aspartokinase polypeptide or a functional variant thereof; and
(f) a Shewanella oneidensis aspartokinase polypeptide or a functional variant thereof.

9. The bacterium of claim 1, wherein the heterologous bacterial aspartate semialdehyde dehydrogenase polypeptide or functional variant thereof is chosen from:
(a) a Mycobacterium smegmatis aspartate semialdehyde dehydrogenase polypeptide or a functional variant thereof;
(b) an Amycolatopsis mediterranei aspartate semialdehyde dehydrogenase polypeptide or a functional variant thereof;
(c) a Streptomyces coelicolor aspartate semialdehyde dehydrogenase polypeptide or a functional variant thereof; and
(d) a Thermobifida fusca aspartate semialdehyde dehydrogenase polypeptide or a functional variant thereof.

10. The bacterium of claim 1, wherein the heterologous bacterial phosphoenolpyruvate carboxylase polypeptide or a functional variant thereof is chosen from:
(a) a Mycobacterium smegmatis phosphoenolpyruvate carboxylase polypeptide or a functional variant thereof;
(b) a Streptomyces coelicolor phosphoenolpyruvate carboxylase polypeptide or a functional variant thereof;
(c) a Thermobifida fusca phosphoenolpyruvate carboxylase polypeptide or a functional variant thereof; and
(d) an Erwinia chrysanthemi phosphoenolpyruvate carboxylase polypeptide or a functional variant thereof.

11. The bacterium of claim 1, wherein the heterologous bacterial pyruvate carboxylase polypeptide or a functional variant thereof is chosen from:
(a) a Mycobacterium smegmatis pyruvate carboxylase polypeptide or a functional variant thereof; and
(b) a Streptomyces coelicolor pyruvate carboxylase polypeptide or a functional variant thereof.

12. The bacterium of claim 1, wherein the bacterium comprises at least two of:
(a) a nucleic acid molecule encoding a heterologous bacterial aspartokinase polypeptide or a functional variant thereof;
(b) a nucleic acid molecule encoding a heterologous bacterial aspartate semialdehyde dehydrogenase polypeptide or a functional variant thereof;
(c) a nucleic acid molecule encoding a heterologous bacterial phosphoenolpyruvate carboxylase polypeptide or a functional variant thereof;
(d) a nucleic acid molecule encoding a heterologous bacterial pyruvate carboxylase polypeptide or a functional variant thereof;
(e) a nucleic acid molecule comprising a sequence encoding a heterologous bacterial dihydrodipicolinate synthase polypeptide or a functional variant thereof;
(f) a nucleic acid molecule comprising a sequence encoding a heterologous bacterial homoserine dehydrogenase polypeptide or a functional variant thereof;
(g) a nucleic acid molecule comprising a sequence encoding a heterologous bacterial homoserine O-acetyltransferase polypeptide or a functional variant thereof;
(h) a nucleic acid molecule comprising a sequence encoding a heterologous bacterial O-acetylhomoserine sulfhydrylase polypeptide or a functional variant thereof;
(i) a nucleic acid molecule comprising a sequence encoding a heterologous bacterial methionine adenosyltransferase polypeptide or a functional variant thereof;
(j) a nucleic acid molecule comprising a sequence encoding a heterologous bacterial mcbR gene product polypeptide or a functional variant thereof;
(k) a nucleic acid molecule comprising a sequence encoding a heterologous bacterial O-succinylhomoserine/acetylhomoserine (thiol)-lyase polypeptide or a functional variant thereof;
(l) a nucleic acid molecule comprising a sequence encoding a heterologous bacterial cystathionine beta-lyase polypeptide or a functional variant thereof;
(m) a nucleic acid molecule comprising a sequence encoding a heterologous bacterial 5-methyltetrahydrofolate homocysteine methyltransferase polypeptide or a functional variant thereof; and
(n) a nucleic acid molecule comprising a sequence encoding a heterologous bacterial 5-methyltetrahydropteroyltriglutamate-homocysteine methyltransferase polypeptide or a functional variant thereof.

13. The bacterium of claim 1, wherein the bacterium comprises at least three of:
(a) a nucleic acid molecule encoding a heterologous bacterial aspartokinase polypeptide or a functional variant thereof;
(b) a nucleic acid molecule encoding a heterologous bacterial aspartate semialdehyde dehydrogenase polypeptide or a functional variant thereof;
(c) a nucleic acid molecule encoding a heterologous bacterial phosphoenolpyruvate carboxylase polypeptide or a functional variant thereof; and
(d) a nucleic acid molecule encoding a heterologous bacterial pyruvate carboxylase polypeptide or a functional variant thereof;
(e) a nucleic acid molecule comprising a sequence encoding a heterologous bacterial dihydrodipicolinate synthase polypeptide or a functional variant thereof;
(f) a nucleic acid molecule comprising a sequence encoding a heterologous bacterial homoserine dehydrogenase polypeptide or a functional variant thereof;
(g) a nucleic acid molecule comprising a sequence encoding a heterologous bacterial homoserine O-acetyltransferase polypeptide or a functional variant thereof;
(h) a nucleic acid molecule comprising a sequence encoding a heterologous bacterial O-acetylhomoserine sulfhydrylase polypeptide or a functional variant thereof;
(i) a nucleic acid molecule comprising a sequence encoding a heterologous bacterial methionine adenosyltransferase polypeptide or a functional variant thereof;
(j) a nucleic acid molecule comprising a sequence encoding a heterologous bacterial mcbR gene product polypeptide or a functional variant thereof;
(k) a nucleic acid molecule comprising a sequence encoding a heterologous bacterial O-succinylhomoserine/acetylhomoserine (thiol)-lyase polypeptide or a functional variant thereof;
(l) a nucleic acid molecule comprising a sequence encoding a heterologous bacterial cystathionine beta-lyase polypeptide or a functional variant thereof;
(m) a nucleic acid molecule comprising a sequence encoding a heterologous bacterial 5-methyltetrahydrofolate homocysteine methyltransferase polypeptide or a functional variant thereof; and
(n) a nucleic acid molecule comprising a sequence encoding a heterologous bacterial 5-methyltetrahydropteroyltriglutamate-homocysteine methyltransferase polypeptide or a functional variant thereof.

14. An Escherichia coli or coryneform bacterium comprising a nucleic acid molecule comprising a sequence encoding a heterologous bacterial dihydrodipicolinate synthase polypeptide or a functional variant thereof.

15. The bacterium of claim 14 wherein the heterologous bacterial dihydrodipicolinate synthase polypeptide or a functional variant thereof is chosen from:
(a) a Mycobacterium smegmatis dihydrodipicolinate synthase polypeptide or a functional variant thereof;
(b) a Streptomyces coelicolor dihydrodipicolinate synthase polypeptide or a functional variant thereof;
(c) a Thermobifida fusca dihydrodipicolinate synthase polypeptide or a functional variant thereof; and
(d) an Erwinia chrysanthemi dihydrodipicolinate synthase polypeptide or a functional variant thereof.

16. An Escherichia coli or coryneform bacterium comprising a nucleic acid molecule comprising a sequence encoding a heterologous bacterial homoserine dehydrogenase polypeptide or a functional variant thereof.

17. The bacterium of claim 16, wherein the heterologous bacterial homoserine dehydrogenase polypeptide is chosen from:
(a) a Mycobacterium smegmatis homoserine dehydrogenase polypeptide or functional variant thereof;
(b) a Streptomyces coelicolor homoserine dehydrogenase polypeptide or a functional variant thereof;
(c) a Thermobifida fusca homoserine dehydrogenase polypeptide or a functional variant thereof; and
(d) an Erwinia chrysanthemi homoserine dehydrogenase polypeptide or a functional variant thereof.

18. An Escherichia coli or coryneform bacterium comprising a nucleic acid molecule comprising a sequence encoding a heterologous bacterial O-homoserine acetyltransferase polypeptide or a functional variant thereof.

19. The bacterium of claim 18, wherein the heterologous bacterial O-homoserine acetyltransferase polypeptide is chosen from:
(a) a Mycobacterium smegmatis O-homoserine acetyltransferase polypeptide or functional variant thereof;
(b) a Streptomyces coelicolor O-homoserine acetyltransferase polypeptide or a functional variant thereof;
(c) a Thermobifida fusca O-homoserine acetyltransferase polypeptide or a functional variant thereof; and
(d) an Erwinia chrysanthemi O-homoserine acetyltransferase polypeptide or a functional variant thereof.

20. An Escherichia coli or coryneform bacterium comprising a nucleic acid molecule that encodes a heterologous bacterial O-acetylhomoserine sulfhydrylase polypeptide or a functional variant thereof.

21. The bacterium of claim 20, wherein the heterologous bacterial O-acetylhomoserine sulfhydrolase polypeptide is chosen from:
(a) a Mycobacterium smegmatis O-acetylhomoserine sulfhydrylase polypeptide or functional variant thereof;
(b) a Streptomyces coelicolor O-acetylhomoserine sulfhydrylase polypeptide or a functional variant thereof; and
(c) a Thermobifida fusca O-acetylhomoserine sulfhydrylase polypeptide or a functional variant thereof.

22. An Escherichia coli or coryneform bacterium comprising a nucleic acid molecule comprising a sequence encoding a heterologous bacterial 5-methyltetrahydrofolate homocysteine methyltransferase polypeptide or a functional variant thereof.

23. The bacterium of claim 22, wherein the heterologous bacterial 5-methyltetrahydrofolate homocysteine methyltransferase polypeptide is chosen from:
(a) a bacterial 5-methyltetrahydrofolate homocysteine methyltransferase polypeptide that is at least 80% identical to SEQ ID No:72 or 73, or a functional variant thereof, from a species of the genus Mycobacterium;
(b) a Streptomyces coelicolor 5-methyltetrahydrofolate homocysteine methyltransferase polypeptide or a functional variant thereof
(c) a Thermobifida fusca 5-methyltetrahydrofolate homocysteine methyltransferase polypeptide or a functional variant thereof; and
(d) a Lactobacillus plantarum 5-methyltetrahydrofolate homocysteine methyltransferase polypeptide or a functional variant thereof.

24. An Escherichia coli or coryneform bacterium comprising a nucleic acid molecule comprising a sequence encoding a heterologous bacterial 5-methyltetrahydropteroyltriglutamate-homocysteine methyltransferase polypeptide or a functional variant thereof.

25. The bacterium of claim 24, wherein the heterologous bacterial 5-methyltetrahydropteroyltriglutamate-homocysteine methyltransferase polypeptide is chosen from:
(a) a bacterial 5-methyltetrahydropteroyltriglutamate-homocysteine methyltransferase polypeptide that is at least 80% identical to SEQ ID No:75 or 76, or a functional variant thereof, from a species of the genus Mycobacterium;
(b) a Streptomyces coelicolor 5-methyltetrahydropteroyltriglutamate-homocysteine methyltransferase polypeptide or a functional variant thereof;
(c) a Thermobifida fusca 5-methyltetrahydropteroyltriglutamate-homocysteine methyltransferase polypeptide or a functional variant thereof; and
(d) a Lactobacillus plantarum 5-methyltetrahydropteroyltriglutamate-homocysteine methyltransferase polypeptide or a functional variant thereof.

26. An Escherichia coli or coryneform bacterium comprising a nucleic acid molecule comprising a sequence encoding a heterologous bacterial methionine adenosyltransferase polypeptide or a functional variant thereof.

27. The bacterium of claim 26, wherein the heterologous bacterial methionine adenosyltransferase polypeptide is chosen from:
(a) a Mycobacterium smegmatis methionine adenosyltransferase polypeptide or functional variant thereof;
(b) a Streptomyces coelicolor methionine adenosyltransferase polypeptide or a functional variant thereof;
(c) a Thermobifida fusca methionine adenosyltransferase polypeptide or a functional variant thereof; and
(d) an Erwinia chrysanthemi methionine adenosyltransferase polypeptide or a functional variant thereof.

28. An Escherichia coli or coryneform bacterium comprising at least two of:
(a) a genetically altered nucleic acid molecule comprising a sequence encoding a bacterial aspartokinase polypeptide or a functional variant thereof;
(b) a genetically altered nucleic acid molecule comprising a sequence encoding a bacterial aspartate semialdehyde dehydrogenase polypeptide or a functional variant thereof;
(c) a genetically altered nucleic acid molecule comprising a sequence encoding a bacterial phosphoenolpyruvate carboxylase polypeptide or a functional variant thereof; and
(d) a genetically altered nucleic acid molecule comprising a sequence encoding a bacterial dihydrodipicolinate synthase polypeptide or a functional variant thereof.

29. The bacterium of claim 28, wherein at least one of the at least two genetically altered nucleic acid molecules encodes a heterologous polypeptide.

30. The bacterium of claim 28, wherein the bacterium comprises (a) and (b), (a) and (c), (a) and (d), (b) and (c), (b) and (d), or (c) and (d).

31. The bacterium of claim 30, wherein the bacterium comprises at least three of (a)-(e).

32. The bacterium of claim 28, wherein the bacterium has reduced activity of one or more of the following polypeptides, relative to a control:
(a) a homoserine dehydrogenase polypeptide;
(b) a homoserine kinase polypeptide; and
(c) a phosphoenolpyruvate carboxykinase polypeptide.

33. The bacterium of claim 32, wherein the bacterium comprises a mutation in an endogenous hom gene or an endogenous thrB gene.

34. The bacterium of claim 32, wherein the bacterium comprises a mutation in an endogenous hom gene and an endogeous thrB gene.

35. The bacterium of claim 32, wherein the bacterium comprises a mutation in an endogenous pck gene.

36. An Escherichia coli or coryneform bacterium comprising at least two of:
(a) a genetically altered nucleic acid molecule comprising a sequence encoding a bacterial phosphoenolpyruvate carboxylase polypeptide or a functional variant thereof;
(b) a genetically altered nucleic acid molecule comprising a sequence encoding a bacterial aspartokinase polypeptide or a functional variant thereof;
(c) a genetically altered nucleic acid molecule comprising a sequence encoding a bacterial aspartate semialdehyde dehydrogenase polypeptide or a functional variant thereof
(d) a genetically altered nucleic acid molecule comprising a sequence encoding a bacterial homoserine dehydrogenase polypeptide or a functional variant thereof;
(e) a genetically altered nucleic acid molecule comprising a sequence encoding a bacterial homoserine O-acetyltransferase polypeptide or a functional variant thereof;
(f) a genetically altered nucleic acid molecule comprising a sequence encoding a bacterial O-acetylhomoserine sulfhydrylase polypeptide or a functional variant thereof;
(g) a genetically altered nucleic acid molecule comprising a sequence encoding a bacterial 5-methyltetrahydrofolate homocysteine methyltransferase polypeptide or a functional variant thereof;
(h) a genetically altered nucleic acid molecule comprising a sequence encoding a bacterial O-succinylhomoserine (thio)-lyase polypeptide or a functional variant thereof;
(i) a genetically altered nucleic acid molecule comprising a sequence encoding a bacterial 5-methyltetrahydropteroyltriglutamate-homocysteine methyltransferase polypeptide or a functional variant thereof;
(j) a genetically altered nucleic acid molecule comprising a sequence encoding a bacterial methionine adenosyltransferase polypeptide or a functional variant thereof;
(k) a genetically altered nucleic acid molecule comprising a sequence encoding a bacterial serine hydroxylmethyltransferase polypeptide or a functional variant thereof; and
(l) a genetically altered nucleic acid molecule comprising a sequence encoding a bacterial cystathionine beta-lyase polypeptide or a functional variant thereof.

37. The bacterium of claim 36, wherein at least one of the at least two genetically altered nucleic acid molecules encodes a heterologous polypeptide.

38. The bacterium of claim 36, wherein the bacterium comprises (a) and at least one of (b), (c), (d), (e), (f), (g), (h), (i), (j), (k), and (l).

39. The bacterium of claim 36, wherein the bacterium comprises (b) and at least one of (c), (d), (e), (f), (g), (h), (i), (j), (k), and (l).

40. The bacterium of claim 36, wherein the bacterium comprises (c) and at least one of (d), (e), (f), (g), (h), (i), (j), (k), and (l).

41. The bacterium of claim 36, wherein the bacterium comprises (d) and at least one of (e), (f), (g), (h), (i), (j), (k), and (l).

42. The bacterium of claim 36, wherein the bacterium comprises (e) and at least one of (f), (g), (h), (i), (j), (k), and (l).

43. The bacterium of claim 36, wherein the bacterium comprises (f) and at least one of (g), (h), (i), (j), (k), and (l).

44. The bacterium of claim 36, wherein the bacterium comprises (g) and at least one of (h), (i), (j), (k), and (l).

45. The bacterium of claim 36, wherein the bacterium comprises (h) and at least one of (i), (j), (k), and (1).

46. The bacterium of claim 36, wherein the bacterium comprises (i) and at least one of (j) (k), and (1).

47. The bacterium of claim 36, wherein the bacterium comprises (j) and at least one of (k), and (l).

48. The bacterium of claim 36, wherein the bacterium comprises (k) and (l).

49. The bacterium of claim 36, wherein the bacterium comprises at least three of (a)-(l).

50. The bacterium of claim 36, wherein the bacterium has reduced activity of one or more of the following polypeptides, relative to a control:
(a) a homoserine kinase polypeptide;
(b) a phosphoenolpyruvate carboxykinase polypeptide;
(c) a homoserine dehydrogenase polypeptide; and
(d) a mcbR gene product polypeptide.

51. The bacterium of claim 50, wherein the bacterium comprises a mutation in an endogenous hom gene, an endogenous thrB gene, an endogenous pck gene, or an endogenous mcbR gene.

52. The bacterium of claim 50, wherein the bacterium comprises a mutation in an endogenous hom gene and an endogeous thrB gene.

53. The bacterium of claim 50, wherein the bacterium comprises a mutation in two or more of an endogenous hom gene, an endogenous thrB gene, an endogenous pck gene, or an endogenous mcbR gene.

54. An Escherichia coli or coryneform bacterium comprising at least two of:
(a) a genetically altered nucleic acid molecule comprising a sequence encoding a bacterial phosphoenolpyruvate carboxylase polypeptide or a functional variant thereof;
(b) a genetically altered nucleic acid molecule comprising a sequence encoding a bacterial aspartokinase polypeptide or a functional variant thereof;
(c) a genetically altered nucleic acid molecule comprising a sequence encoding a bacterial aspartate semialdehyde dehydrogenase polypeptide or a functional variant thereof;
(d) a genetically altered nucleic acid molecule comprising a sequence encoding a bacterial homoserine dehydrogenase polypeptide or a functional variant thereof.

55. The bacterium of claim 54, wherein at least one of the at least two polypeptides encodes a heterologous polypeptide.

56. The bacterium of claim 54, wherein the bacterium comprises (a) and (b), (a) and (c), (a) and (d), (b) and (c), (b) and (d), or (c) and (d).

57. The bacterium of claim 54, wherein the bacterium comprises at least three of (a)-(d).

58. The bacterium of claim 54, wherein the bacterium has reduced activity of one or more of the following polypeptides, relative to a control:
(a) a phosphoenolpyruvate carboxykinase polypeptide; and
(b) a mcbR gene product polypeptide.

59. The bacterium of claim 58, wherein the bacterium comprises a mutation in an endogenous pck gene or an endogenous mcbR gene.

60. The bacterium of claim 58, wherein the bacterium comprises a mutation in an endogenous pck gene and an endogenous mcbR gene.

61. A method of producing an amino acid or a related metabolite, the method comprising:
cultivating a bacterium according to claim 1 under conditions that allow the amino acid the metabolite to be produced, and collecting a composition that comprises the amino acid or related metabolite from the culture.

62. The method of claim 61, further comprising fractionating at least a portion of the culture to obtain a fraction enriched in the amino acid or the metabolite.

63. A method for producing L-lysine or a related metabolite, the method comprising:
cultivating a bacterium according to claim 1 or 28 under conditions that allow L-lysine to be produced, and collecting a composition that comprises the amino acid or related metabolite from the culture.

64. The method of claim 63, further comprising fractionating at least a portion of the culture to obtain a fraction enriched in L-lysine.

65. A method for producing methionine or S-adenosylmethionine, the method comprising:
cultivating a bacterium according to claim 36 under conditions that allow methionine or S-adenosylmethionine to be produced, and collecting a composition that comprises the methionine or S-adenosylmethionine from the culture.

66. The method of claim 65, further comprising fractionating at least a portion of the culture to obtain a fraction enriched in methionine or S-adenosylmethionine.

67. A method for producing isoleucine or threonine, the method comprising:
cultivating a bacterium according to claim 54 under conditions that allow isoleucine or threonine to be produced, and collecting a composition that comprises the a isoleucine or threonine from the culture.

68. The method of claim 67, further comprising fractionating at least a portion of the culture to obtain a fraction enriched in isoleucine or threonine.

69. An isolated nucleic acid encoding a variant bacterial protein, wherein the bacterial protein regulates the production of an amino acid from the aspartic acid family of amino acids or related metabolites, and wherein the variant protein has enhanced activity, relative to a wild type form of the protein

70. The nucleic acid of claim 69, wherein the bacterial protein regulates the production of an amino acid from the aspartic acid family of amino acids or related metabolites, and wherein the variant protein has reduced feedback inhibition by S-adenosylmethionine relative to a wild type form of the protein.

71. An isolated nucleic acid encoding a variant of a bacterial protein, wherein the bacterial protein comprises the following amino acid sequence:
(SEQ ID NO:360)
G₁-X₂-K₃-X₄-X₅-X₆-X₇-X₈-X₉-X₁₀-X₁₁-X₁₂-X₁₃-X₁₃ₐ-
X_{13b}-X_{13c}-X_{13d}-X₁₃ₑ-X_{13f}-X_{13g}-X₁₃ₕ-X₁₃ᵢ-X₁₃ⱼ-X₁₃ₖ-
X₁₃ₗ-F₁₄-X₁₅-Z₁₆-X₁₇-X₁₈-X₁₉-X₂₀-X₂₁-X₂₁ₐ-X_{21b}-
X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-
X₂₁ₘ-X₂₁ₙ-X₂₁ₒ-X₂₁ₚ-X_{21q}-X₂₁ᵣ-X₂₁ₛ-X₂₁ₜ-D_{22,}
wherein each of X₂, X₄—X₁₃, X₁₅, and X₁₇—X₂₀ is, independently, any amino acid, wherein each of X₁₃ₐ—X₁₃ₗ is, independently, any amino acid or absent, wherein each of X₂₁ₐ—X₂₁ₜ is, independently, any amino acid or absent, and wherein Z₁₆ is selected from valine, aspartate, glycine, isoleucine, and leucine;
wherein the variant bacterial protein comprises an amino acid change at one or more of G₁, K₃, F₁₄, Z₁₆, or D₂₂ of SEQ ID NO:360).

72. The nucleic acid of claim 71, wherein feedback inhibition of the variant of the bacterial protein by S-adenosylmethionine is reduced relative to the bacterial protein.

73. The nucleic acid of claim 71, wherein the amino acid change is a change to an alanine.

74. A polypeptide encoded by the nucleic acid of claim 69.

75. A polypeptide encoded by the nucleic acid of claim 71.

76. A bacterium comprising the nucleic acid of claim 69.

77. A bacterium comprising the nucleic acid of claim 71.

78. A method for producing an amino acid or a related metabolite, the method comprising:
cultivating a genetically modified bacterium comprising the nucleic acid of claim 69 under conditions in which the nucleic acid is expressed and that allow the amino acid to be produced, and collecting a composition that comprises the amino acid or related metabolite from the culture.

79. A method for producing an amino acid or a related metabolite, the method comprising:
cultivating a genetically modified bacterium comprising the nucleic acid of claim 71 under conditions in which the nucleic acid is expressed and that allow the amino acid to be produced, and collecting a composition that comprises the amino acid or related metabolite from the culture.

80. An isolated nucleic acid encoding a variant bacterial homoserine O-acetyltransferase, wherein the variant homoserine O-acetyltransferase is a variant of a homoserine O-acetyltransferase comprising the following amino acid sequence:
(SEQ ID NO:360)
G₁-X₂-K₃-X₄-X₅-X₆-X₇-X₈-X₉-X₁₀-X₁₁-X₁₂-X₁₃-X₁₃ₐ-
X_{13b}-X_{13c}-X_{13d}-X₁₃ₑ-X_{13f}-X_{13g}-X₁₃ₕ-X₁₃ᵢ-X₁₃ⱼ-X₁₃ₖ-
X₁₃ₗ-F₁₄-X₁₅-Z₁₆-X₁₇-X₁₈-X₁₉-X₂₀-X₂₁-X₂₁ₐ-X_{21b}-
X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-
X₂₁ₘ-X₂₁ₙ-X₂₁ₒ-X₂₁ₚ-X_{21q}-X₂₁ᵣ-X₂₁ₛ-X₂₁ₜ-D_{22,}
wherein each of X₂, X₄—X₁₃, X₁₅, and X₁₇—X₂₀ is, independently, any amino acid, wherein each of X₁₃ₐ—X₁₃ₗ is, independently, any amino acid or absent, wherein each of X₂₁ₐ—X₂₁ₜ is, independently, any amino acid or absent, and wherein Z₁₆ is selected from valine, aspartate, glycine, isoleucine, and leucine;
wherein the variant homoserine O-acetyltransferase comprises an amino acid change at one or more of G₁, K₃, F₁₄, Z₁₆, or D₂₂ of SEQ ID NO:360.

81. An isolated nucleic acid encoding a variant bacterial O-acetylhomoserine sulfhydrylase, wherein the variant O-acetylhomoserine sulfhydrylase is a variant of an O-acetylhomoserine sulfhydrylase comprising the following amino acid sequence:
(SEQ ID NO:360)
G₁-X₂-K₃-X₄-X₅-X₆-X₇-X₈-X₉-X₁₀-X₁₁-X₁₂-X₁₃-X₁₃ₐ-
X_{13b}-X_{13c}-X_{13d}-X₁₃ₑ-X_{13f}-X_{13g}-X₁₃ₕ-X₁₃ᵢ-X₁₃ⱼ-X₁₃ₖ-
X₁₃ₗ-F₁₄-X₁₅-Z₁₆-X₁₇-X₁₈-X₁₉-X₂₀-X₂₁-X₂₁ₐ-X_{21b}-
X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-
X₂₁ₘ-X₂₁ₙ-X₂₁ₒ-X₂₁ₚ-X_{21q}-X₂₁ᵣ-X₂₁ₛ-X₂₁ₜ-D_{22,}
wherein X is any amino acid, wherein each of X₁₃ₐ—X₁₃ₗ is, independently, any amino acid or absent, wherein each of X₂₁ₐ—X₂₁ₜ is, independently, any amino acid or absent, and wherein Z₁₆ is selected from valine, aspartate, glycine, isoleucine, and leucine;
wherein the variant O-acetylhomoserine sulfhydrylase comprises an amino acid change at one or more of G₁, K₃, F₁₄, Z₁₆, or D₂₂ of SEQ ID NO:360.

82. An isolated nucleic acid encoding a variant bacterial mcbR gene product, wherein the variant mcbR gene product is a variant of an mcbR gene product comprising the following amino acid sequence:
(SEQ ID NO:360)
G₁-X₂-K₃-X₄-X₅-X₆-X₇-X₈-X₉-X₁₀-X₁₁-X₁₂-X₁₃-X₁₃ₐ-
X_{13b}-X_{13c}-X_{13d}-X₁₃ₑ-X_{13f}-X_{13g}-X₁₃ₕ-X₁₃ᵢ-X₁₃ⱼ-X₁₃ₖ-
X₁₃ₗ-F₁₄-X₁₅-Z₁₆-X₁₇-X₁₈-X₁₉-X₂₀-X₂₁-X₂₁ₐ-X_{21b}-
X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-
X₂₁ₘ-X₂₁ₙ-X₂₁ₒ-X₂₁ₚ-X_{21q}-X₂₁ᵣ-X₂₁ₛ-X₂₁ₜ-D_{22,}
wherein each of X₂, X₄—X₁₃, X₁₅, and X₁₇—X₂₀ is, independently, any amino acid, wherein each of X₁₃ₐ—X₁₃ₗ is, independently, any amino acid or absent, wherein each of X₂₁ₐ—X₂₁ₜ is, independently, any amino acid or absent, and wherein Z₁₆ is selected from valine, aspartate, glycine, isoleucine, and leucine;
wherein the variant mcbR gene product comprises an amino acid change at one or more of G₁, K₃, F₁₄, Z₁₆, or D₂₂ of SEQ ID NO:360

83. An isolated nucleic acid encoding a variant bacterial aspartokinase, wherein the variant aspartokinase is a variant of an aspartokinase comprising the following amino acid sequence:
(SEQ ID NO:360)
G₁-X₂-K₃-X₄-X₅-X₆-X₇-X₈-X₉-X₁₀-X₁₁-X₁₂-X₁₃-X₁₃ₐ-
X_{13b}-X_{13c}-X_{13d}-X₁₃ₑ-X_{13f}-X_{13g}-X₁₃ₕ-X₁₃ᵢ-X₁₃ⱼ-X₁₃ₖ-
X₁₃ₗ-F₁₄-X₁₅-Z₁₆-X₁₇-X₁₈-X₁₉-X₂₀-X₂₁-X₂₁ₐ-X_{21b}-
X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-
X₂₁ₘ-X₂₁ₙ-X₂₁ₒ-X₂₁ₚ-X_{21q}-X₂₁ᵣ-X₂₁ₛ-X₂₁ₜ-D_{22,}
wherein each of X₂, X₄—X₁₃, X₁₅, and X₁₇—X₂₀ is, independently, any amino acid, wherein each of X₁₃ₐ—X₁₃ₗ is, independently, any amino acid or absent, wherein each of X₂₁ₐ—-X₂₁ₜ is, independently, any amino acid or absent, and wherein Z₁₆ is selected from valine, aspartate, glycine, isoleucine, and leucine;
wherein the variant aspartokinase comprises an amino acid change at one or more of G₁, K₃, F₁₄, Z₁₆, or D₂₂ of SEQ ID NO:360.

84. An isolated nucleic acid encoding a variant bacterial O-succinylhomoserine (thiol)-lyase, wherein the variant O-succinylhomoserine (thiol)-lyase is a variant of an O-succinylhomoserine (thiol)-lyase comprising the following amino acid sequence:
(SEQ ID NO:360)
G₁-X₂-K₃-X₄-X₅-X₆-X₇-X₈-X₉-X₁₀-X₁₁-X₁₂-X₁₃-X₁₃ₐ-
X_{13b}-X_{13c}-X_{13d}-X₁₃ₑ-X_{13f}-X_{13g}-X₁₃ₕ-X₁₃ᵢ-X₁₃ⱼ-X₁₃ₖ-
X₁₃ₗ-F₁₄-X₁₅-Z₁₆-X₁₇-X₁₈-X₁₉-X₂₀-X₂₁-X₂₁ₐ-X_{21b}-
X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-
X₂₁ₘ-X₂₁ₙ-X₂₁ₒ-X₂₁ₚ-X_{21q}-X₂₁ᵣ-X₂₁ₛ-X₂₁ₜ-D_{22,}
wherein each of X₂, X₄—X₁₃, X₁₅, and X₁₇—X₂₀ is, independently, any amino acid, wherein each of X₁₃ₐ—X₁₃ₗ is, independently, any amino acid or absent, wherein each of X₂₁ₐ—X₂₁ₜ is, independently, any amino acid or absent, and wherein Z₁₆ is selected from valine, aspartate, glycine, isoleucine, and leucine;
wherein the variant O-succinylhomoserine (thiol)-lyase comprises an amino acid change at one or more of G₁, K₃, F₁₄, Z₁₆, or D₂₂ of SEQ ID NO:360.

85. An isolated nucleic acid encoding a variant bacterial cystathionine beta-lyase, wherein the variant cystathionine beta-lyase is a variant of a cystathionine beta-lyase comprising the following amino acid sequence:
(SEQ ID NO:360)
G₁-X₂-K₃-X₄-X₅-X₆-X₇-X₈-X₉-X₁₀-X₁₁-X₁₂-X₁₃-X₁₃ₐ-
X_{13b}-X_{13c}-X_{13d}-X₁₃ₑ-X_{13f}-X_{13g}-X₁₃ₕ-X₁₃ᵢ-X₁₃ⱼ-X₁₃ₖ-
X₁₃ₗ-F₁₄-X₁₅-Z₁₆-X₁₇-X₁₈-X₁₉-X₂₀-X₂₁-X₂₁ₐ-X_{21b}-
X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-
X₂₁ₘ-X₂₁ₙ-X₂₁ₒ-X₂₁ₚ-X_{21q}-X₂₁ᵣ-X₂₁ₛ-X₂₁ₜ-D_{22,}
wherein each of X₂, X₄—X₁₃, X₁₅, and X₁₇—X₂₀ is, independently, any amino acid, wherein each of X₁₃ₐ—X₁₃ₗ is, independently, any amino acid or absent, wherein each of X₂₁ₐ—X₂₁ₜ is, independently, any amino acid or absent, and wherein Z₁₆ is selected from valine, aspartate, glycine, isoleucine, and leucine;
wherein the variant cystathionine beta-lyase comprises an amino acid change at one or more of G₁, K₃, F₁₄, Z₁₆, or D₂₂ of SEQ ID NO:360.

86. An isolated nucleic acid encoding a variant bacterial 5-methyltetrahydrofolate homocysteine methyltransferase, wherein the variant 5-methyltetrahydrofolate homocysteine methyltransferase is a variant of a 5-methyltetrahydrofolate homocysteine methyltransferase comprising the following amino acid sequence:
(SEQ ID NO:362)
G₁-X₂-K₃-X₄-X₅-X₆-X₇-X₈-X₉-X₁₀-X₁₁-X₁₂-X₁₃-X₁₃ₐ-
X_{13b}-X_{13c}-X_{13d}-X₁₃ₑ-X_{13f}-X_{13g}-X₁₃ₕ-X₁₃ᵢ-X₁₃ⱼ-X₁₃ₖ-
X₁₃ₗ-F₁₄-X₁₅-Z₁₆
wherein each of X₂, X₄—X₁₃, X₁₅, and X₁₅—X₁₆ is, independently,wherein X is any amino acid, wherein each of X_{13a—X}₁₃ₗ is, independently, any amino acid or absent, and wherein Z₁₆ is selected from valine, aspartate, glycine, isoleucine, and leucine;
wherein the variant homocysteine methyltransferase comprises an amino acid change at one or more of G₁, K₃, F₁₄, or Z₁₆, of SEQ ID NO:362.

87. An isolated nucleic acid encoding a variant bacterial S-adenosylmethionine synthetase, wherein the variant S-adenosylmethionine synthetase is a variant of an S-adenosylmethionine synthetase comprising the following amino acid sequence:
(SEQ ID NO:360)
G₁-X₂-K₃-X₄-X₅-X₆-X₇-X₈-X₉-X₁₀-X₁₁-X₁₂-X₁₃-X₁₃ₐ-
X_{13b}-X_{13c}-X_{13d}-X₁₃ₑ-X_{13f}-X_{13g}-X₁₃ₕ-X₁₃ᵢ-X₁₃ⱼ-X₁₃ₖ-
X₁₃ₗ-F₁₄-X₁₅-Z₁₆-X₁₇-X₁₈-X₁₉-X₂₀-X₂₁-X₂₁ₐ-X_{21b}-
X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-
X₂₁ₘ-X₂₁ₙ-X₂₁ₒ-X₂₁ₚ-X_{21q}-X₂₁ᵣ-X₂₁ₛ-X₂₁ₜ-D_{22,}
wherein each of X₂, X₄—X₁₃, X₁₅, and X₁₇—X₂₀ is, independently, any amino acid, wherein each of X₁₃ₐ—X₁₃ₗ is, independently, any amino acid or absent, wherein each of X₂₁ₐ—X₂₁ₜ is, independently, any amino acid or absent, and wherein Z₁₆ is selected from valine, aspartate, glycine, isoleucine, and leucine;
wherein the variant S-adenosylmethionine synthetase comprises an amino acid change at one or more of G₁, K₃, F₁₄, Z₁₆, or D₂₂ of SEQ ID NO:360.

88. A bacterium comprising two or more of the following:
a nucleic acid encoding a variant bacterial homoserine O-acetyltransferase with reduced feedback inhibition relative to a wild-type form of the homoserine O-acetyltransferase;
a nucleic acid encoding a variant bacterial O-acetylhomoserine sulfhydrylase with reduced feedback inhibition relative to a wild-type form of the O-acetylhomoserine sulfhydrylase;
a nucleic acid encoding a variant bacterial McbR gene product with reduced feedback inhibition relative to a wild-type form of the McbR gene product;
a nucleic acid encoding a variant bacterial aspartokinase with reduced feedback inhibition relative to a wild-type form of the aspartokinase;
a nucleic acid encoding a variant bacterial O-succinylhomoserine (thiol)-lyase with reduced feedback inhibition relative to a wild-type form of the O-succinylhomoserine (thiol)-lyase;
a nucleic acid encoding a variant bacterial cystathionine beta-lyase with reduced feedback inhibition relative to a wild-type form of the cystathionine beta-lyase;
a nucleic acid encoding a variant bacterial homocysteine methyltransferase with reduced feedback inhibition relative to a wild-type form of the 5-methyltetrahydrofolate homocysteine methyltransferase; and
a nucleic acid encoding a variant bacterial S-adenosylmethionine synthetase with reduced feedback inhibition relative to a wild-type form of the S-adenosylmethionine synthetase.

89. A bacterium comprising two or more of the following:
(a) a nucleic acid encoding a variant bacterial homoserine O-acetyltransferase, wherein the variant homoserine O-acetyltransferase is a variant of a homoserine O-acetyltransferase comprising the following amino acid sequence:
(SEQ ID NO:360)
G₁-X₂-K₃-X₄-X₅-X₆-X₇-X₈-X₉-X₁₀-X₁₁-X₁₂-X₁₃-X₁₃ₐ-
X_{13b}-X_{13c}-X_{13d}-X₁₃ₑ-X_{13f}-X_{13g}-X₁₃ₕ-X₁₃ᵢ-X₁₃ⱼ-X₁₃ₖ-
X₁₃ₗ-F₁₄-X₁₅-Z₁₆-X₁₇-X₁₈-X₁₉-X₂₀-X₂₁-X₂₁ₐ-X_{21b}-
X_{21c}-X_{21d}-X₂₁ₑ-X_{21f}-X_{21g}-X₂₁ₕ-X₂₁ᵢ-X₂₁ⱼ-X₂₁ₖ-X₂₁ₗ-
X₂₁ₘ-X₂₁ₙ-X₂₁ₒ-X₂₁ₚ-X_{21q}-X₂₁ᵣ-X₂₁ₛ-X₂₁ₜ-D_{22,}
wherein each of X₂, X₄—X₁₃, X₁₅, and X₁₇—X₂₀ is, independently, any amino acid, wherein each of X₁₃ₐ—X₁₃ₗ is, independently, any amino acid or absent, wherein each of X₂₁ₐ—X₂₁ₜ is, independently, any amino acid or absent, and wherein Z₁₆ is selected from valine, aspartate, glycine, isoleucine, and leucine;
wherein the variant homoserine O-acetyltransferase comprises an amino acid change at one or more of G₁, K₃, F₁₄, Z₁₆, or D₂₂ of SEQ ID NO:360;
(b) a nucleic acid encoding a variant bacterial O-acetylhomoserine sulfhydrylase, wherein the variant O-acetylhomoserine sulfhydrylase is a variant of an O-acetylhomoserine sulfhydrylase comprising the following amino acid sequence:
(SEQ ID NO:360)
G₁-X₂K₃-X₄-X₅-X₆-X₇-X₈-X₉-X₁₀-X₁₁-X₁₂-X₁₃-X₁₃ₐ-
X_{13b}-X_{13c}-X_{13d}-X₁₃ₑ-X_{13f}-X_{13g}-X₁₃ₕ-X₁₃ᵢ-X₁₃ⱼ-
X₁₃ₖ-X₁₃ₗ-F₁₄-X₁₅-Z₁₆-X₁₇-X₁₈-X₁₉-X₂₀-X₂₁-X₂₁ₐ-
X_{21b}-X_{21c}-X_{21d}-X₂₁ₑ-X_{21f}-X_{21g}-X₂₁ₕ-X₂₁ᵢ-X₂₁ⱼ-X₂₁ₖ-
X₂₁ₗ-X₂₁ₘ-X₂₁ₙ-X₂₁ₒ-X₂₁ₚ-X_{21q}-X₂₁ᵣ-X₂₁ₛ-X₂₁ₜ-D_{22,}
wherein each of X₂, X₄—X₁₃, X₁₅, and X₁₇—X₂₀ is, independently, any amino acid, wherein each of X₁₃ₐ—X₁₃ₗ is, independently, any amino acid or absent, wherein each of X₂₁ₐ—X₂₁ₜ is, independently, any amino acid or absent, and wherein Z₁₆ is selected from valine, aspartate, glycine, isoleucine, and leucine;
wherein the variant O-acetylhomoserine sulfhydrylase comprises an amino acid change at one or more of G₁, K₃, F₁₄, Z₁₆, or D₂₂ of SEQ ID NO:360; and
(c) a nucleic acid encoding a variant bacterial O-acetylhomoserine sulfhydrylase, wherein the variant O-acetylhomoserine sulfhydrylase is a variant of a O-acetylhomoserine sulfhydrylase comprising the following amino acid sequence:
L₁-X₂—X₃-G₄-G₅-X₆—F₇—X₈—X₉—X₁₀—X₁₁ (SEQ ID NO:361), wherein X is any amino acid, wherein X₈ is selected from valine, leucine, isoleucine, and aspartate, and wherein X₁₁ is selected from valine, leucine, isoleucine, phenylalanine, and methionine; wherein the variant of the bacterial protein comprises an amino acid change at one or more of L₁, G₄, X₈, X₁₁ of SEQ ID NO:361.

90. A bacterium comprising two or more of the following:
(a) a nucleic acid encoding a variant bacterial homoserine O-acetyltransferase, wherein the variant homoserine O-acetyltransferase is a C. glutamicum homoserine O-acetyltransferase comprising an amino acid change in one or more of the following residues of SEQ ID NO:212 Glycine 231, Lysine 233, Phenylalanine 251, and Valine 253;
(b) a nucleic acid encoding a variant bacterial homoserine O-acetyltransferase, wherein the variant homoserine O-acetyltransferase is a T. fusca homoserine O-acetyltransferase comprising an amino acid change in one or more of the following residues of SEQ ID NO:24: Glycine 81, Aspartate 287, Phenylalanine 269;
(c) a nucleic acid encoding a variant bacterial homoserine O-acetyltransferase, wherein the variant homoserine O-acetyltransferase is an E. coli homoserine O-acetyltransferase comprising an amino acid change at Glutamate 252 of SEQ ID NO:213;
(d) a nucleic acid encoding a variant bacterial homoserine O-acetyltransferase, wherein the variant homoserine O-acetyltransferase is a mycobacterial homoserine O-acetyltransferase comprising an amino acid change in a residue corresponding to one or more of the following residues of M. leprae homoserine O-acetyltransferase set forth in SEQ ID NO: 23: Glycine 73, Aspartate 278, and Tyrosine 260;
(e) a nucleic acid encoding a variant bacterial homoserine O-acetyltransferase, wherein the variant homoserine O-acetyltransferase is an M. tuberculosis homoserine O-acetyltransferase comprising an amino acid change in one or more of the following residues of SEQ ID NO:22: Glycine 73, Tyrosine 260, and Aspartate 278;
(f) a nucleic acid encoding a variant bacterial O-acetylhomoserine sulfhydrylase, wherein the variant O-acetylhomoserine sulfhydrylase is a C. glutamicum O-acetylhomoserine sulfhydrylase comprising an amino acid change in one or more of the following residues of SEQ ID NO:214: Glycine 227, Leucine 229, Aspartate 231, Glycine 232, Glycine 233, Phenylalanine 235, Aspartate 236, Valine 239, Phenylalanine 368, Aspartate 370, Aspartate 383, Glycine 346, and Lycine 348; and
(g) a nucleic acid encoding a variant bacterial O-acetylhomoserine sulfhydrylase, wherein the variant O-acetylhomoserine sulfhydrylase is a T. fusca O-acetylhomoserine sulfhydrylase comprising an amino acid change in one or more of the following residues of SEQ ID NO:25: Glycine 240, Aspartate 244, Phenylalanine 379, and Aspartate 394.

91. A bacterium comprising a nucleic acid encoding an episomal homoserine O-acetyltransferase, or a variant thereof, and an episomal O-acetylhomoserine sulfhydrylase, or a variant thereof.

92. The bacterium of claim 91, wherein the episomal homoserine O-acetyltransferase and the episomal O-acetylhomoserine sulfhydrylase are of a different species than the bacterium.

93. A method for the preparation of animal feed additives containing an aspartate-derived amino acid(s) comprising:
(a) cultivating a bacterium according to any of claims 1, 28, 36, and 54 under conditions that allow the aspartate-derived amino acid(s) to be produced;
(b) collecting a composition that comprises at least a portion of the aspartate-derived amino acid(s) that result from cultivating said bacterium;
(c) concentrating the collected composition to enrich for the aspartate-derived amino acid(s); and
(d) optionally, adding one or more substances to obtain the desired animal feed additive.

94. The method of claim 93, wherein the bacterium is Escherichia coli or a coryneform bacterium.

95. The method of claim 94, wherein the bacterium is Corynebacterium glutamicum.

96. The method of claim 93, wherein the aspartate-derived amino acid one or more of lysine, methionine, threonine or isoleucine.
